SEQUENCE LISTING (22 sequences)

SEQ ID NO: 1 - protein, 7 aa, Simian virus 40
PKKKRKV

SEQ ID NO: 2 - protein, 16 aa, Unknown
  source: /note=“Description of Unknown Nucleoplasmin bipartite NLS sequence”
KRPAATKKAGQAKKKK

SEQ ID NO: 3 - protein, 9 aa, Unknown
  source: /note=“Description of Unknown C-myc NLS sequence”
PAAKRVKLD

SEQ ID NO: 4 - protein, 11 aa, Unknown
  source: /note=“Description of Unknown C-myc NLS sequence”
RQRRNELKRSP

SEQ ID NO: 5 - protein, 38 aa, Homo sapiens
NQSSNFGPMKGGNFGGRSSGPYGGGGQYFAKPRNQGGY

SEQ ID NO: 6 - protein, 42 aa, Unknown
  source: /note=“Description of Unknown IBB domain from importin-alpha sequence”
RMRIZFKNKGKDTAELRRRRVEVSVELRKAKKDEQILKRRNV

SEQ ID NO: 7 - protein, 8 aa, Unknown
  source: /note=“Description of Unknown Myoma T protein sequence”
VSRKRPRP

SEQ ID NO: 8 - protein, 8 aa, Unknown
  source: /note=“Description of Unknown Myoma T protein sequence”
PPKKARED

SEQ ID NO: 9 - protein, 8 aa, Homo sapiens
PQPKKKPL

SEQ ID NO: 10 - protein, 12 aa, Mus musculus
SALIKKKKKMAP

SEQ ID NO: 11 - protein, 5 aa, Influenza virus
DRLRR

SEQ ID NO: 12 - protein, 7 aa, Influenza virus
PKQKKRK

SEQ ID NO: 13 - protein, 10 aa, Hepatitis delta virus
RKLKKKIKKL

SEQ ID NO: 14 - protein, 10 aa, Mus musculus
REKKKFLKRR

SEQ ID NO: 15 - protein, 20 aa, Homo sapiens
KRKGDEVDGVDEVAKKKSKK

SEQ ID NO: 16 - protein, 17 aa, Homo sapiens
RKCLQAGMNLEARKTKK

SEQ ID NO: 17 - protein, 12 aa, Artificial Sequence
  source: /note=“Description of Artificial Sequence Synthetic peptide”
  MOD_RES (2)..(2): Ahx
  MOD_RES (5)..(5): Ahx
  MOD_RES (8)..(8): Ahx
  MOD_RES (11)..(11): Ahx
RXRRXRRXRRXR

SEQ ID NO: 18 - protein, 288 aa, Artificial Sequence
  source: /note=“Description of Artificial Sequence Synthetic polypeptide”
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 19 - protein, 183 aa, Artificial Sequence
  source: /note=“Description of Artificial Sequence Synthetic polypeptide”
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 20 - DNA, 34 bp, Artificial Sequence
  source: /note=“Description of Artificial Sequence Synthetic primer”
ccctccaaggtttgtctgcagccattcgaggtgc

SEQ ID NO: 21 - DNA, 34 bp, Artificial Sequence
  source: /note=“Description of Artificial Sequence Synthetic primer”
ccctccaaggtttgtctgcagccattcgaggtgt

SEQ ID NO: 22 - DNA, 21 bp, Artificial Sequence
  source: /note=“Description of Artificial Sequence Synthetic primer”
tcgggcagcctgcatgatctt

(19) United States
(12) Patent Application Publication
(10) Pub. No.: US 2018/0100201 A1
(43) Pub. Date: Apr. 12, 2018

(54) TUMOR AND MICROENVIRONMENT GENE EXPRESSION, COMPOSITIONS OF MATTER AND METHODS OF USE THEREOF

(71) Applicants: The Broad Institute Inc., Cambridge, MA (US); Massachusetts Institute of Technology, Cambridge, MA (US); Dana-Farber Cancer Institute, Inc., Boston, MA (US); The General Hospital Corporation, Boston, MA (US)

(72) Inventors: Levi A. Garraway, Newton, MA (US); Benjamin Izar, Boston, MA (US); Sanjay Prakadan, Somverville, MA (US); Aviv Regev, Cambridge, MA (US); Orit Rozenblatt-Rosen, Sharon, MA (US); Alexander K. Shalek, Lexington, MA (US); Mario Suva, Boston, MA (US); Itay Tirosh, Brookline, MA (US); Andrew Venteicher, Boston, MA (US); Marc H. Wadsworth II, Cambridge, MA (US); Bradley Bernstein, Cambridge, MA (US); Anuraag Parikh, Boston, MA (US); Sidharth Puram, Cambridge, MA (US)

(21) Appl. No.: 15/844,601

(22) Filed: Dec. 17, 2017

Related U.S. Application Data
(63) Continuation-in-part of application No. PCT/US2016/040015, filed on Jun. 29, 2016.
(60) Provisional application No. 62/186,227, filed on Jun. 29, 2015.
(60) Provisional application No. 62/286,850, filed on Jan. 25, 2016.

(51) Int. Cl. C12Q 1/6886

(52) U.S. Cl. CPC C12Q 1/6886; C12Q 2600/158; C12Q 2600/112; C12Q 2600/106

(57) ABSTRACT

This invention relates generally to compositions and methods for identifying genes and gene networks that respond to, modulate, control or otherwise influence tumors and tissues, including cells and cell types of the tumors and tissues, and malignant, microenvironmental, or immunologic states of the tumor cells and tissues. The invention also relates to methods of diagnosing, prognosing and/or staging of tumors, tissues and cells, and provides compositions and methods of modulating expression of genes and gene networks of tumors, tissues and cells, as well as methods of identifying, designing and selecting appropriate treatment regimens. The invention also relates to the modulation of complement activity to shift cellular immunity and obtain an effective therapeutic response.

### RELATED APPLICATIONS AND INCORPORATION BY REFERENCE

This application continuation-in-part application of international patent application Serial No. PCT/US2016/040015 filed Jun. 29, 2016, which published as PCT Publication No. WO2017/004153 on Jan. 5, 2017, which claims priority and benefit of U.S. provisional application Ser. No. 62/186,227, filed Jun. 29, 2015 and 62/286,850, filed Jan. 25, 2016.

The foregoing application, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under grant numbers CA180922, CA14051, DO20839 and CA112962 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Aug. 29, 2016, is named 48009_99_2013_SL.txt and is 10 bytes in size.

### FIELD OF THE INVENTION

The present invention generally relates to the methods of identifying and using gene expression profiles representative of malignant, microenvironmental, or immunologic states of tumors, and use of such profiles for diagnosing, prognosing and/or staging of melanomas and designing and selecting appropriate treatment regimens.

### BACKGROUND OF THE INVENTION

Tumors are complex ecosystems defined by spatiotemporal interactions between heterogeneous cell types, including malignant, immune and stromal cells (1). Each tumor's cellular composition, as well as the interplay between these components, may exert critical roles in cancer development (2). However, the specific components, their salient biological functions, and the means by which they collectively define tumor behavior remain incompletely characterized.

Tumor cellular diversity poses both challenges and opportunities for cancer therapy. This is most clearly demonstrated by the remarkable but varied clinical efficacy achieved in malignant melanoma with targeted therapies and immunotherapies. First, immune checkpoint inhibitors produce substantial clinical responses in some patients with metastatic melanomas (3-7); however, the genomic and molecular determinants of response to these agents remain poorly understood. Although tumor neoantigens and PD-L1 expression clearly contribute (8-10), it is likely that other factors from subsets of malignant cells, the microenvironment, and tumor-infiltrating lymphocytes (TILs) also play essential roles (11). Second, melanomas that harbor the BRAFV600E mutation are commonly treated with RAF/MEK-inhibition prior to or following immune checkpoint inhibition. Although this regimen improves survival, virtually all patients eventually develop resistance to these drugs (12,13). Unfortunately, no targeted therapy currently exists for patients whose tumors lack BRAF mutations—including NRAS mutant tumors, those with inactivating NF1 mutations, or rarer events (e.g., RAF fusions). Collectively, these factors highlight the need for a deeper understanding of melanoma composition and its impact on clinical course.

The next wave of therapeutic advances in cancer will likely be accelerated by emerging technologies that systematically assess the malignant, microenvironmental, and immunologic states most likely to inform treatment response and resistance. An ideal approach would assess salient cellular heterogeneity by quantifying variation in oncogenic signaling pathways, drug-resistant tumor cell subsets, and the spectrum of immune, stromal and other cell states that may inform immunotherapy response. Toward this end, emerging single-cell genomic approaches enable detailed evaluation of genetic and transcriptional features present in 100s-11000s of individual cells per tumor (14-16). In principle, this approach may provide a comprehensive means to identify all major cellular components simultaneously, determine their individual genomic and molecular states (15), and ascertain which of these features may predict or explain clinical responses to anticancer agents.

Intra-tumoral heterogeneity contributes to therapy failure and disease progression in cancer. Tumor cells vary in proliferation, stemness, invasion, apoptosis, chemoresistance and metabolism (72). Various factors may contribute to this heterogeneity. On the one hand, in the genetic model of cancer, distinct tumor subclones are generated by branched genetic evolution of cancer cells; on the other hand, it is also becoming increasingly clear that certain cancers display diversity due to features of normal tissue organization. From this perspective, non-genetic determinants, related to developmental pathways and epigenetic programs, such as those associated with the self-renewal of tissue stem cells and their differentiation into specialized cell types, contribute to tumor functional heterogeneity (73,74). In particular, in a hierarchical developmental model of cancer, cancer stem cells (CSC) have the unique capacity to self-renew and to generate non-tumorigenic differentiated cancer cells. This model is still controversial, but—if correct—has important practical implications for patient management (75,76). Pioneering studies in leukemias have indeed demonstrated that targeting stem cell programs or triggering cellular differentiation can override genetic alterations and yield clinical benefit (72,77).

Relating the genetic and non-genetic models of cancer heterogeneity, especially in solid human tumors, has been limited due to technical challenges. Analysis of human tumor genomes has shed light on the genetic model, but is typically performed in bulk and does not inform us on the concomitant functional states of cancer cells. Conversely, various markers have been used to isolate candidate CSCs across different human malignancies, and to demonstrate their capacity to propagate tumors in mouse xenograft experiments (72,78-80). For example, in the field of human gliomas, candidate CSCs have been isolated in high-grade (WHO grades III-IV) lesions, using either combinations of cell surface markers such as CD133, SSEA-1, A2B5, CD44 and α-6 integrin or by in vitro selection and expansion of gliomaspheres in serum-free conditions (75,76,78,80-83). However, these functional approaches have generated controversy, as they require in vitro or in vivo selection in animal models with results dependent on xenogeneic environments that are very different from the native human tumor milieu. In addition, these methods do not interrogate the relative contribution of genetic mutations to the observed phenotypes (which can limit reproducibility) and do not allow an unbiased analysis of cellular states in situ in human patients (72). It also remains largely unknown if candidate CSC-like cells described in human high-grade tumors are aberrantly generated during glioma progression by dedifferentiation of mature glial cells or if gliomas contain CSC-like cells early in their development—as grade II lesions—a question central for our understanding of the initial steps of gliomagenesis (84). Thus, it is critical to cancer biology to develop a framework that allows the unbiased analysis of cellular programs at the single-cell level and across different genetic clones in human tumors, in situ, and at each stage of clinical progression, especially early in their development.

The present invention provides novel methods of identifying gene expression profiles representative of malignant, microenvironmental, or immunologic states of tumors and tissues, and of cells and cell types which they comprise. The invention further provides methods of diagnosing, prognosing and/or staging of tumors, tissues and cells. The invention also provides compositions and methods of modulating expression of genes and gene networks of tumors, tissues and cells, as well as methods of identifying, designing and selecting appropriate treatment regimens.

Citation or identification of any document in this application is not an admission that such document is available as prior art to the present invention.

### SUMMARY OF THE INVENTION

The invention relates to gene expression signatures and networks of tumors and tissues, as well as multicellular ecosystems of tumors and tissues and the cells and cell type which they comprise. Tumors are multicellular assemblies that encompass many distinct genotypic and phenotypic states. The invention provides methods of characterizing components, functions and interactions of tumors and tissues and the cells which they comprise. Single-cell RNA-seq was applied to thousands of malignant and non-malignant cells derived from melanomas, gliomas, head and neck cancer, brain metastases of breast cancer, and tumors in The Cancer Genome Atlas (TCGA) to examine tumor ecosystems.

The invention provides signature genes, gene products, and expression profiles of signature genes, gene networks, and gene products of tumors and component cells. The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases. In one embodiment, the patient is suffering from melanoma. The signature genes, gene products, and expression profiles are useful to identify components of tumors and tissues and states of such components, such as, without limitation, neoplastic cells, malignant cells, stem cells, immune cells, and malignant, microenvironmental, or immunologic states of such component cells.

Using single cell analysis in cancers including melanoma, glioma, brain metastases of breast cancer, and head and neck squamous cell carcinoma (HNSCC), as well as analyzing tumors in The Cancer Genome Atlas (TCGA), applicants have determined novel gene signature patterns and therapeutic targets.

In one aspect, the present invention provides for a method of diagnosing, prognosing and/or staging a condition or disorder having an immunological state, comprising detecting a first level of expression, activity and/or function of one or more signature genes or one or more products of one or more signature genes in one or more cell(s) of the disorder and comparing the detected level to a control level of signature gene or gene product expression, activity and/or function, wherein the one or more signature genes comprise a component of the complement system, and wherein a difference in the detected level and the control level indicates an immunologic state of the condition or disorder. The one or more signature genes may comprise C1S, C1R, C3, C4A, CFB, C1QA, C1QB, C1QC, CD46, CD55, CD59 or SERPING1. The immunologic state of the condition or disorder may be characterized by the presence or absence of immune cells comprising myeloid-derived suppressor cells myeloid-derived suppressor cells (MDSC), macrophages, dendritic cells (DC), natural killer cells (NK), T cells and/or B cells, wherein expression of the one or more signature genes correlates to the abundance of the immune cells. The condition or disorder may be an autoimmune diseases, inflammatory diseases, infections or cancer. Not being bound by a theory, expression of a complement signature gene in a specific cell type, such as, but not limited to cancer associated fibroblasts (CAF), microglia, macrophages indicate the abundance of other cell types, such as T cells and B cells. The inflammatory disease may be a pathogenic or non-pathogenic Th17 response. The cancer may be Non-Hodgkin's Lymphoma (NHL), clear cell Renal Cell Carcinoma (ccRCC), melanoma, sarcoma, leukemia or a cancer of the bladder, colon, brain, breast, head and neck, endometrium, lung, ovary, pancreas or prostate. The cancer may be a recurrent cancer. The cancer may be from a patient who progressed through chemotherapy. The one or more signature genes may be a gene that indicates the abundance of T cells. The one or more signature genes may be detected in CAFs. The one or more signature genes may be C1S, C1R, C3, C4A, CFB, or SERPING1. The one or more signature genes may be detected in macrophages. The one or more signature genes may be C1QA, C1QB or C1QC. The one or more signature genes may be a gene that indicates the abundance of B cells. The one or more signature genes may be detected in CAFs. The one or more signature genes may be C7 or C3. The one or more signature genes may be a gene that indicates the abundance of macrophages. The one or more signature genes may be detected in CAFs. The one or more signature genes may be C1S, C1R or CFB. The level or expression of the one or more signature genes may be determined by single-cell RNA sequencing. The single-cell RNA sequencing may be single nucleus RNA-Seq. The level of expression, activity and/or function of one or more signature genes may be determined by the level of expression of one or more products encoded by one or more signature genes in one or more cell(s). The level of expression of one or more products encoded by one or more signature genes may be determined by a colorimetric assay or absorbance assay. The level of expression, activity and/or function of one or more signature genes or one or more products of one or more signature genes in one or more cell(s) may be determined by deconvolution of bulk expression data.

In another aspect, the present invention provides for a method of treating or enhancing treatment of condition or disorder having an immunological state, which comprises administering an agent that increases or decreases the function, activity and/or expression of one or more signature genes or one or more products of one or more signature genes in one or more cell(s) of the disorder, wherein the one or more signature genes comprise a component of the complement system. In one embodiment administering of the agent increases or decreases the abundance of an immune cell. The immune cells may be myeloid-derived suppressor cells (MDSC), macrophages, dendritic cells (DC), natural killer cells (NK), T cells, B cells or any combination therewith. The agent may increase or decrease the function, activity and/or expression of C1S, C1R, C3, C4A, CFB, C1QA, C1QB, C1QC, CD46, CD55, CD59, C5 or SERPING1(CFI). Not being bound by a theory, immune cells, such as, but not limited to T cells may be inhibitory to complement activity and have low cytolytic activity, wherein activation of complement may increase the cytolytic activity of the T cells.

The condition or disorder may be cancer and the agent may decrease the function, activity and/or expression of a complement defense or protection molecule including CD46. CD55 or CD59, whereby malignant cells have enhanced susceptibility to killing by complement activation. Not being bound by a theory, increasing complement activation, either through complement component activation, or inhibition of protection molecules or inhibitors of complement activation, unexpectedly results in an increase in immune cell abundance. The agent may be a CRISPR-Cas system that activates expression of the component of the complement system. The agent may be a CRISPR-Cas system that targets the component of the complement system, whereby the component gene is knocked out or expression is decreased. The agent may be an isolated natural product, whereby the component of the complement system is activated. The agent may be a metalloproteinase, whereby a component of the complement system is directly cleaved. The agent may be a serine protease, whereby a component of the complement system is directly cleaved. The agent may be a therapeutic antibody or fragment thereof. The cancer may be Non-Hodgkin's Lymphoma (NHL), clear cell Renal Cell Carcinoma (ccRCC), melanoma, sarcoma, leukemia or a cancer of the bladder, colon, brain, breast, head and neck, endometrium, lung, ovary, pancreas or prostate.

In one embodiment, wherein the condition or disorder is cancer, administering of the agent results in killing of a malignant cell. Not being bound by a theory, malignant cells uniformly express the complement protection molecules CD46, CD55 and CD59, thus malignant cells are protected against killing by complement. Not being bound by a theory, targeting of these protection molecules provides for killing of the malignant cells by complement. In one embodiment, a protection molecule is targeted for inhibition and complement is activated, thus increasing the killing of the malignant cells by complement. Not being bound by a theory, the protection molecules are surface proteins that can be targeted for inhibition by therapeutic antibodies or binding compounds that inhibit their activity. Not being bound by a theory, the surface molecules may be targeted by CAR T cells, thus preferentially killing malignant cells expressing the protection molecules. Not being bound by a theory, the surface molecules may be targeted by antibody drug conjugates, thus preferentially killing malignant cells expressing the protection molecules.

Using human oligodendrogliomas as a model, the inventors have profiled single cells from six patient tumors by RNA-seq and reconstructed their transcriptional architecture and related it to genetic mutations. It was surprisingly found that most cancer cells are differentiated along two specialized glial programs, while a rare subpopulation of cells is undifferentiated and associated with a neural stem cell/progenitor expression program. Surprisingly, cellular proliferation was highly enriched in this rare subpopulation, consistent with a model where a cancer stem cell/progenitor compartment is primarily responsible for fueling growth of oligodendrogliomas in humans. Analysis of sub-clonal genetic events shows that distinct clones within tumors span a similar cellular hierarchy, suggesting that the architecture of oligodendroglioma is primarily dictated by non-genetic developmental programs. These results provide unprecedented insight into the cellular composition of brain tumors at single-cell resolution and may help harmonize the cancer stem cell and the genetic models of cancer, with critical implications for disease management.

In an aspect, the invention relates to a method of treating glioma, comprising administering to a subject having glioma a therapeutically effective amount of an agent capable of reducing the expression or inhibiting the activity of one or more stem cell or progenitor cell signature genes or polypeptides; or capable of targeting or binding to one or more cell surface exposed stem cell or progenitor cell signature polypeptides. The agent may be capable of targeting or binding to one or more cell surface exposed stem cell or progenitor cell signature polypeptides and may be a CAR T cell capable of targeting or binding to one or more cell surface exposed stem cell or progenitor cell signature polypeptides.

In a further aspect, the invention relates to a method of treating glioma, comprising administering to a subject having glioma a therapeutically effective amount of an agent capable of inducing the expression or increasing the activity of one or more astrocyte and/or oligodendrocyte cell signature genes or polypeptides.

In an aspect, the invention relates to a method of treating glioma or enhancing treatment of glioma, which comprises administering an agent that increases or decreases expression of or the function of one or more signature genes or one or more products of one or more signature genes in one or more cell(s) of the glioma, wherein the one or more signature genes or one or more products of one or more signature genes comprises a signature gene as defined herein elsewhere. In certain embodiments astrocyte and/or oligodendrocyte signature gene expression or function/activity is increased. In certain embodiments, stem/progenitor cell signature gene expression or function/activity is decreased.

In certain embodiments, the level of expression, activity and/or function of one or more signature genes is determined by the level of expression of one or more products encoded by one or more signature genes in one or more cell(s) of the glioma. In certain embodiments, the level of expression of one or more products encoded by one or more signature genes is determined by a colorimetric assay or absorbance assay. In certain embodiments, the level of expression, activity and/or function of one or more signature genes or one or more products of one or more signature genes in one or more cell(s) of the glioma is determined by deconvolution of the bulk expression properties of a tumor.

As used herein, the term glioma has its ordinary meaning in the art. By means of further guidance, glioma refers to a tumor arising in the brain or spine, and is typically derived from or associated with glial cells. In certain embodiments, glioma as referred to herein includes without limitation oligodendrogliomas (derived from oligodendrocytes), ependymomas (derived from ependymal cells), astrocytomas (derived from astrocytes, and including glioblastoma (glioblastoma multiforme or grade IVV astrocytoma)), brainstem glioma (develops in the brain stem), optic nerve glioma (develops in or around the optic nerve), or mixed gliomas (such as oligoastrocytomas, containing cells from different types of glia). In a particular embodiment, glioma refers to oligodendroglioma.

In certain embodiments, said glioma is low grade glioma. In certain embodiments, said glioma is high grade glioma. In certain embodiments, said glioma is grade I glioma. In certain embodiments, said glioma is grade II glioma. In certain embodiments, said glioma is grade III glioma. In certain embodiments, said glioma is grade IV glioma. In a preferred embodiment, said glioma is low grade glioma, or grade II glioma. Staging or grading or cancer in general and glioma in particular is well known in the art. By means of example, glioma may be graded according to the grading system of the World Health Organization (e.g. WHO grade II oligodendroglioma). In certain embodiments, glioma is primary glioma. In certain embodiments, glioma is metastatic (or secondary) glioma. In certain embodiments, glioma is recurrent glioma.

In certain embodiments, glioma as referred to herein is characterized by IDH1 and/or IDH2 (isocytrate dehydrogenase 1/2) mutations. In certain embodiments, the IDH1 mutation is R132H. In certain embodiments glioma as referred to herein is characterized by deletion of chromosome arms 1p and/or 19q. In certain embodiments, glioma as referred to herein is characterized by IDH1 and/or IDH2 mutations, such as IDH1 R132H mutation, and co-deletion of chromosome arms 1p and/or 19q. In certain embodiments, glioma is characterized by CIC (Protein capicua homolog) mutation. In certain embodiments, glioma as referred to herein is characterized by IDH1 and/or IDH2 mutations, such as IDH1 R132H mutation, and CIC mutation. In certain embodiments, glioma as referred to herein is characterized by deletion of chromosome arms 1p and/or 19q, and CIC mutation. In certain embodiments, glioma as referred to herein is characterized by IDH1 and/or IDH2 mutations, such as IDH1 R132H mutation, co-deletion of chromosome arms 1p and/or 19q, and CIC mutation. In certain embodiments, glioma as referred to herein is characterized by mutations in one or more gene selected from the group consisting of FAM120B, FGR1B, TP18, ESD, MTMR4, TUBB4A, H2AFV, EEF1B2, TMEM5, CEP170, EIF2AK2, SEC63, PTP4A1, RP11-556N21.1, ZEB2, DNAJC4, ZNF292, and ANKRD36, one or more of which mutations may be present in the same cell or different cells of the tumor and may be present in the same cell or different cells of the tumor together with IDH1 and/or IDH2 mutations, such as IDH1 R132H mutation, co-deletion of chromosome arms 1p and/or 9q, and/or CIC mutation.

It will be understood that when referring to mutations in glioma, such mutations may be present in all or part of the tumor, such as for instance in all cells or in particular cell populations of the tumor. Hence a mutation is present or detected in at least part or the tumor or in at least part of the tumor cells. Mutation as referred to herein may refer to functional alteration of the affected gene, such as activation or inactivation of the gene or gene product, which may or may not be epigenetically.

In certain embodiments, the subject to be treated has not previously received chemotherapy and/or radiotherapy. In certain embodiments, the subject to be treated has previously received chemotherapy and/or radiotherapy.

In certain embodiments, treatment as referred to herein may comprise inducing differentiation of stem cells or progenitor cells comprised by or comprised in the glioma. In certain embodiments, said differentiation comprises induction of expression or activity of one or more astrocyte and/or oligodendrocyte signature genes or polypeptides in the stem cells or progenitor cells. In certain embodiments, treatment as referred to herein comprises reducing the viability of or rendering non-viable stem cells or progenitor cells comprised by or comprised in the glioma.

In an aspect, the invention relates to a method of diagnosing, prognosing, or stratifying or staging glioma, comprising determining expression or activity of one or more stem cell or progenitor cell signature genes or polypeptides in cells comprised by the glioma.

In an aspect, the invention relates to a method of diagnosing, prognosing, or stratifying or staging glioma, comprising determining expression or activity of one or more astrocyte signature genes or polypeptides in cells comprised by the glioma.

In an aspect, the invention relates to a method of diagnosing, prognosing, or stratifying or staging glioma, comprising determining expression or activity of one or more oligodendrocyte signature genes or polypeptides in cells comprised by the glioma.

In an aspect, the invention relates to a method of diagnosing, prognosing and/or staging a glioma, comprising detecting a first level of expression, activity and/or function of one or more signature genes or one or more products of one or more signature genes in one or more cell(s), population of cells or subpopulation of cells of the glioma and comparing the detected level to a control level of signature gene or gene product expression, activity and/or function, wherein a difference in the detected level and the control level indicates a malignant, microenvironmental, or immunologic state of the glioma.

In certain embodiments, such method comprises determining the relative expression level of one or more stem cell or progenitor cell signature genes or polypeptides compared to one or more astrocyte and/or oligodendrocyte signature genes or polypeptides in the cells comprised by or comprised in the glioma. In certain embodiments, such method comprises determining the fraction of the cells comprised by the glioma, which express one or more stem cell or progenitor cell signature genes or polypeptides. In certain embodiments, such method comprises determining the fraction of the cells comprised by the glioma, which express one or more astrocyte signature genes or polypeptides. In certain embodiments, such method comprises determining the fraction of the cells comprised by the glioma, which express one or more oligodendrocyte signature genes or polypeptides. In certain embodiments, such method comprises determining the fraction of the cells comprised by the glioma, which express one or more stem/progenitor cell, astrocyte, and oligodendrocyte signature genes or polypeptides. It will be understood that when referring to stem/progenitor cell, astrocyte, or oligodendrocyte signatures as referred to herein, such signatures may be specific for particular tumor cells or tumor cell (sub)populations having certain stem/progenitor, astrocyte, or oligodendrocyte characteristics, such as for instance as determined histologically or by means of identification of particular signatures characteristic of normal (i.e. non-cancerous) stem/progenitor, astrocyte, or oligodendrocyte cells. In certain embodiments, stem or progenitor cells as referred to herein refers to neural stem or progenitor cells.

In an aspect, the invention relates to a method of diagnosing, prognosing, stratifying or staging glioma, comprising identifying cells comprised by the glioma, which express one or more of CX3CR1, CD14, CD53, CD68, CD74, FCGR2A, HLA-DRA, or CSF1R, and/or one or more of MOBP, OPALIN, MBP, PLLP, CLDN11, MOG, or PLP1. In certain embodiments, these cells do not contain mutations, such as oncogenic mutations, in particular copy number variations (CNV). In certain embodiments, these cells do not contain IDH1 and/or IDH2 mutations, such as IDH1 R132H mutation, co-deletion of chromosome arms 1p and/or 19q, and CIC mutations. In certain embodiments, these cells do not contain mutations in FAM120B, FGR1B, TP18, ESD, MTMR4, TUBB4A, H2AFV, EEF1B2, TMEM5, CEP170, EIF2AK2, SEC63, PTP4A 1, RP11-556N21.1, ZEB2, DNAJC4, ZNF292, and ANKRD36.

In an aspect, the invention relates to a method of identifying a therapeutic for glioma, comprising administering to a glioma cell, preferably in vitro, a candidate therapeutic and monitoring expression or activity of one or more stem cell or progenitor cell signature genes or polypeptides. In an aspect, the invention relates to a method of identifying a therapeutic for glioma, comprising administering to a glioma cell, preferably in vitro, a candidate therapeutic and monitoring expression or activity of one or more astrocyte cell signature genes or polypeptides. In an aspect, the invention relates to a method of identifying a therapeutic for glioma, comprising administering to a glioma cell, preferably in vitro, a candidate therapeutic and monitoring expression or activity of one or more oligodendrocyte signature genes or polypeptides. In an aspect, the invention relates to a method of identifying a therapeutic for glioma, comprising administering to a glioma cell, preferably in vitro, a candidate therapeutic and monitoring expression or activity of one or more stem cell or progenitor cell, astrocyte, and/or oligodendrocyte signature genes or polypeptides. As used herein, the term therapeutic refers to any agent suitable for therapy, as defined herein elsewhere.

In certain embodiments, reduction in expression or activity of said one or more stem cell or progenitor cell signature genes or polypeptides is indicative of a therapeutic effect. In certain embodiments, increase in expression or activity of said one or more astrocyte signature genes or polypeptides is indicative of a therapeutic effect. In certain embodiments, increase in expression or activity of said one or more oligodendrocyte signature genes or polypeptides is indicative of a therapeutic effect. In certain embodiments, reduction in expression or activity of said one or more stem cell or progenitor cell signature genes or polypeptides and concomitant increase in expression or activity of said one or more astrocyte and/or oligodendrocyte signature genes or polypeptides is indicative of a therapeutic effect.

In an aspect, the invention relates to a method of monitoring glioma treatment or evaluating glioma treatment efficacy, comprising determining expression or activity of one or more stem cell or progenitor cell signature genes or polypeptides in cells comprised by the glioma. In an aspect, the invention relates to a method of monitoring glioma treatment or evaluating glioma treatment efficacy, comprising determining expression or activity of one or more astrocyte signature genes or polypeptides in cells comprised by the glioma. In an aspect, the invention relates to a method of monitoring glioma treatment or evaluating glioma treatment efficacy, comprising determining expression or activity of one or more oligodendrocyte signature genes or polypeptides in cells comprised by the glioma. In an aspect, the invention relates to a method of monitoring glioma treatment or evaluating glioma treatment efficacy, comprising determining expression or activity of one or more stem cell or progenitor cell, astrocyte, and/or oligodendrocyte signature genes or polypeptides in cells comprised by the glioma.

In an aspect, the invention relates to a method for monitoring a subject undergoing a treatment or therapy for glioma comprising detecting a level of expression, activity and/or function of one or more signature genes or one or more products of one or more signature genes of the glioma (e.g. tumor stem/progenitor cell, astrocyte, and/or oligodendrocyte; as defined herein elsewhere) in the absence of the treatment or therapy and comparing the level of expression, activity and/or function of one or more signature genes or one or more products of one or more signature genes in the presence of the treatment or therapy, wherein a difference in the level of expression, activity and/or function of one or more signature genes or one or more products of one or more signature genes in the presence of the treatment or therapy indicates whether the patient is responsive to the treatment or therapy. In certain embodiments, the treatment or therapy modulates expression of one or more signature genes that indicates cell cycle state.

In certain embodiments, said monitoring methods comprises determining the relative expression level of one or more stem cell or progenitor cell signature genes or polypeptides compared to one or more astrocyte and/or oligodendrocyte signature genes or polypeptides in the cells comprised by the glioma. For instance, a decrease in expression of stem cell or progenitor cell signature genes or polypeptides and/or an increase of astrocyte and/or oligodendrocyte cell signature genes or polypeptides may be indicative of therapeutic effect.

In certain embodiments, said monitoring methods comprises determining the fraction of the cells comprised by the glioma, which express one or more stem cell or progenitor cell signature genes or polypeptides. In certain embodiments, said method comprises determining the fraction of the cells comprised by the glioma, which express one or more astrocyte cell signature genes or polypeptides. In certain embodiments, said method comprises determining the fraction of the cells comprised by the glioma, which express one or more oligodendrocyte cell signature genes or polypeptides. In certain embodiments, said method comprises determining the fraction of the cells comprised by the glioma, which express one or more stem cell or progenitor cell, astrocyte, and/or oligodendrocyte signature genes or polypeptides.

In certain embodiments of the invention, the stem cell or progenitor cell signature genes or polypeptides are not oligodendrocyte precursor cell signature genes or polypeptides.

In certain embodiments of the invention, the one or more stem cell or progenitor cell signature gene is selected from SOX4, CCND2, SOX11, RBM6, HNRNPH1, HNRNPL, PTMA, TRA2A, SET, C6orf62, PTPRS, CHD7, CD24, H3F3B, C14orf23, NFIB, SRGAP2C, STMN2, SOX2, TFDP2, CORO1C, EIF4B, FBLIM1, SPDYE7P, TCF4, ORC6, SPDYE1, NCRUPAR. BAZ2B, NELL2, OPHN1, SPHKAP, RAB42, LOH12CR2, ASCL1, BOC, ZBTB8A, ZNF793, TOX3, EGFR, PGM5P2, EEF1A1, MALAT1, TATDN3, CCL5, EVI2A, LYZ, POU5F1, FBXO27, CAMK2N1, NEK5, PABPC1, AFMID, QPCTL, MBOAT1, HAPLN1, LOC90834, LRTOMT, GATM-AS1, AZGP1, RAMP2-AS1, SPDYE5, TNFAIP8L1, which are preferably expressed or upregulated.

In certain embodiments of the invention, the one or more stem cell or progenitor cell signature gene or polypeptide is selected from the group consisting of SOX4, SOX11, SOX2, NFIB, ASCL1, CDH7, CD24, BOC, and TCF4, which are preferably expressed or upregulated.

In certain embodiments of the invention, the one or more stem cell or progenitor cell signature gene or polypeptide is selected from the group consisting of SOX4, CCND2, SOX11, CDH7, CD24, NFIB, SOX2, TCF4, ASCL1, BOC, and EGFR, which are preferably expressed or upregulated.

In certain embodiments of the invention, the one or more stem cell or progenitor cell signature gene or polypeptide is selected from the group consisting of SOX11, SOX4, NFIB TCF4, SOX2, CDH7, BOC, and CCND2, which are preferably expressed or upregulated.

In certain embodiments of the invention, the one or more stem cell or progenitor cell signature gene or polypeptide is selected from the group consisting of SOX11, PTMA, NFIB, CCND2, SOX4, TCF4, CD24, CHD7, and SOX2, which are preferably expressed or upregulated.

In certain embodiments of the invention, the one or more stem cell or progenitor cell signature gene or polypeptide is selected from the group consisting of SOX2, SOX4, SOX11, MSI1, TERF2, CTNNB1, USP22, BRD3, CCND2, and PTEN, which are preferably expressed or upregulated.

In certain embodiments of the invention, the one or more stem cell or progenitor cell signature gene or polypeptide is selected from the SOX4, PTPRS, NFIB, CCND2, RBM6, SET, BAZ2B, TRA2A, which are preferably expressed or upregulated.

In certain embodiments of the invention, the stem cell or progenitor cell signature gene is selected from the group consisting of SOX2, SOX4, SOX6, SOX9, SOX11, CDH7, TCF4, BAZ2B, DCX, PDGFRA, DKK3, GABBR2, CA12, PLTP, IGFBP7, FABP7, LGR4, and ATP1A2, which are preferably expressed or upregulated.

In certain embodiments of the invention, the tumor stem cell or progenitor cell expresses or has an increased expression of one or more of NEDD4L, KCNQ1OT1, UGDH-AS1, ORC4, IGFBPL1, SHISA9, ASTN2, DCX, METTL21A, TMEM212, OPHN1, NRXN3, NREP, ARHGEF26-AS1, ODF2L, ABCC9, PEG10, SOX9, SOX4, TCF4, CHD7, UGT8, DLX5, XKR9, DLX6-AS1, SOX11, PDGFRA, DLX1, NPY, L2HGDH, PTPRS, GLIPR1L2, REXO1L1, CCL5, CTDSP2, SOX2, MAB21L3, TP53I11, GATS, ZFHX4, BAZ2B, DCLK2, GRIA2, LPAL2, CREBBP, MARCH6, PGM5P2, RERE, SPC25, GRIK3, CCDC88A, PVRIG, BRD3, GRIA3, MOXD1, SNTG1, TAGLN3, GSG1, DLX2, ATCAY, NUMA1, LMO1, POGZ, BPTF, CHRM3, RUFY3, SOX6, RPS11, TNFAIP8L1, FOXN3, DAPK1, DLL3, HERC2P4, TFDP2, GTF2IP1, DLX6, IGF1R, MLL3, NCAM1, CHL1, GNRHR2, CLIP3, FBLIM1, MATR3, CCNG2, NEK5, ETV1, KAT6B, SRRM2, FOXP1, DDX17, GOSR1, GATAD2B, MAP4K4, MIAT, CD24, ZNF638, HNRNPH1, BRD8, MLL, PCMTD1, AGPAT4, YPEL1, TNIK, PUM1, RFTN2, NNAT, MALAT1, GAD1, ZNF37BP, IRGQ, FXYD6, PRRC2B, FAM110B, YPEL3, ZMIZ1, CLASP1, SYNE2, BASP1, LYZ, ROCK1P1, DPY19L2P2, RSF1, HIP1, KANSL1, ELAVL4, TET3, ZEB2, ZBTB8A, MTSS1, TNRC6B, FOXO3, ANKRD12, MEIS3, JMJD1C, RICTOR, MEST.

In certain embodiments of the invention, the tumor stem cell or progenitor cell expresses or has an increased expression of one or more of MAD2L1, ZWINT, MLF1IP, RRM2, CCNA2, TPX2, UBE2T, KIF11, MELK, NCAPG, MKI67, NUSAP1, CDK1, HMGB2, NCAPH, KIAA0101, FANCI, NUF2, TACC3, PRC1, CDCA5, FOXM1, CENPF, KIFC1, TOP2A, KIF2C, SMC2, AURKB, FAM64A, ASPM, DIAPH3, UBE2C, BUB1B, NDC80, ASF1B, KIF22, TK1, FANCD2, CASC5, GTSE1, RRM1, RACGAP1, TYMS, BIRC5, PBK, SPAG5, KIF23, TMPO, KIF15, DHFR, H2AFZ, ANLN, ORC6, ARHGAP11A, ESCO2, KIF4A, RNASEH2A, RAD51AP1, KIAA1524, SMC4, CENPN, KIF18B, VRK1, CCNB2, CKS1B, CKAP2L, SHCBP1, HIST1H1B, SGOL1, HIST1H3B, CENPM, CCNB1, BUB1, CENPK, HMGN2, ECT2, HMGB1, UHRF1, NCAPD2, HJURP, PKMYT1, MYBL2, CDC45, CDCA2, DLGAP5, TUBB, MCM10, ATAD2, MXD3, TUBA1B, SGOL2, DTYMK, CDC25C, TROAP, DTL, CDCA3, H2AFX, LIG1, TRIP13, HAUS8, KIF20B, NCAPG2, CDKN3, MIS18BP1, BRCA1, PLK4, CENPW, CDC20, SKA3, HIST1H4C, LMNB1, CDCA8, PLK1, RFC3, CENPO, DNMT1, EXO1, OIP5, CHAF1A, CENPE, POC1A, DEK, NUCKS1, MCM7, MIS18A, DEPDC1B, CHEK1, SPC24, GMNN, PTTG1, EZH2, MCM4, FEN1, GINS1, TTK, CDC6, RAD51, C19orf48, KIF20A, CKAP2, CDCA4, RFC5, SKA1, CENPQ, FANCA, PCNA, RFC4, PARP2, TMEM194A, FBXO5, TIMELESS, PSMC3IP, HIRIP3, POLA1, RANBP1, KIF18A, TCF19, USP1, LRR1, GGH, HMMR, CKS2, DNAJC9, SAE1, ITGB3BP, TMEM106C, FANCG, KPNA2, NCAPD3, HELLS, TMEM48, CBX5, SNRPB, KNTC1, NASP, MCM3, ZWILCH, RPA3, CHTF18, ANP32E, HIST1H3I, POLA2, MZT1, MCM2, DEPDC1, DUT, POLE, PHIP, PTMA, CSE1L, DSCC1, CDC7, HMGB3, TUBB4B, STMN1, RPA2, RCC1, CENPH, GINS2, EXOSC9, NCAPH2, NUDT15, SPC25, HNRNPA2B1, MND1, DSN1, MASTL, RAD21, PHGDH, ZNF331, RANGAP1, SAPCD2, PARPBP, ANP32B, SMC1A, NEK2, BARD1, NIF3L1, PRR11, HNRNPD, MCM5, SMC3, FAM111A, POLD1, CDK2, FUS, PHF19, ARHGAP33, NUP205, CDC25B, PA2G4, NUDT1, CHEK2, WDR34, H2AFY, HAUS1, BUB3, CHAF1B, PRIM2, CCDC34, POLE2, PRPS2, RFWD3, UBR7, CCNE2, RAN, DDX11, NUP50, CACYBP, HNRNPAB, DBF4, TMSB15A, AURKA, MAD2L2, GINS3, ASRGL1, PPIF, CKAP5, UBE2S, LMNB2, POLD3, TEX30, SUV39H1, CCP110, WHSC1, MCM6, ACYP1, GNG4, PRIM1, NSMCE4A, EXOSC8, COMMD4, SNRPD1, HAT1, H2AFV, CMC2, SSRP1, HIST1H1E, RBMX, LBR, RPL39L, EMP2, CENPL, CEP78, TRAIP, COPS3, LSM4, RBBP8, HIST1H1C, RPA1, RAD1, NUP210, HSPB11, RFC2, ACTL6A, SRRT, NUP107, GPN3, LSM3, SUV39H2, POLR2D, HAUS5, WDR76, LSM5, NXT1, TUBG1, C16orf59, REEP4, BTG3, RNASEH2B, TUBB6, PPIA, RBL1, ARL6IP6, COX17, SYNE2, GUSB, MSH5, CRNDE, DDX39A, SUPT16H, HNRNPUL1, POLE3, HAUS4, IDH2, H1FX, DCP2, NUP188, MPHOSPH9, PPIG, MAGOHB, RIF1, MLH1, MSH2, SNRNP40, HADH, GABPB1, NUDC, PHTF2, NUP85, NUP35, SKP2, THOC3, ANAPC11, TFAM, AKR1B1, ILF2, TMEM237, RAD54B, SMPD4, HMGN1, CBX3, TPRKB, GGCT, FBL, RFC1, CCT5, PRKDC, CDK5RAP2, SRSF2, CEP112, LDHA, SRSF3, HSP90AA1, SRSF7, HAUS6, CCHCR1, CEP57, HMGA1, UCHL5, C1orf174, CTPS1, ACOT7, SNHG1, PSMC3, ZNF93, PCM1, SFPQ, RMI1, NUP37, DCK, AHI1, SVIP, CHCHD2, ZNF714, XRCC5, NFATC2IP, SLC25A5, WRAP53, PSIP1, MRPS6, NT5DC2, NOP58.

In certain embodiments, the one or more stem cell or progenitor cell signature gene is selected from the group consisting of SOX4, SOX11, HNRNPH1, PTMA, PTPRS, CHD7, CD24, SOX2, TFDP2, FBLIM1, TCF4, ORC6, BAZ2B, OPHN1, ZBTB8A, PGM5P2, MALAT1, CCL5, LYZ, NEK5, TNFAIP8L1, which are preferably expressed or upregulated.

In certain embodiments, the one or more stem cell or progenitor cell signature gene is selected from the group consisting of CCND2, RBM6, HNRNPL, TRA2A, SET, C6orf62, H3F3B, C14orf23, NFIB, SRGAP2C, STMN2, CORO1C, EIF4B, SPDYE7P, SPDYE1, NCRUPAR, NELL2, SPHKAP, RAB42, LOH12CR2, ASCL1, BOC, ZNF793, TOX3, EGFR, EEF1A1, TATDN3, EVI2A, POU5F1, FBXO27, CAMK2N1, PABPC1, AFMID, QPCTL, MBOAT1, HAPLN1, LOC90834, LRTOMT, GATM-AS1, AZGP1, RAMP2-AS1, SPDYE5, which are preferably expressed or upregulated.

In certain embodiments, the stem cell or progenitor cell signature gene is selected from one or more of the group consisting of SOX4, SOX11, HNRNPH1, PTMA, PTPRS, CHD7, CD24, SOX2, TFDP2, FBLIM1, TCF4, ORC6, BAZ2B, OPHN1, ZBTB8A, PGM5P2, MALAT1, CCL5, LYZ, NEK5, TNFAIP8L1; and one or more of the group consisting of CCND2, RBM6, HNRNPL, TRA2A, SET, C6orf62, H3F3B, C14orf23, NFIB, SRGAP2C, STMN2, CORO1C, EIF4B, SPDYE7P, SPDYE1, NCRUPAR, NELL2, SPHKAP, RAB42, LOH12CR2, ASCL1, BOC, ZNF793, TOX3, EGFR, EEF1A1, TATDN3, EVI2A, POU5F1, FBXO27, CAMK2N1, PABPC1, AFMID, QPCTL, MBOAT1, HAPLN1, LOC90834, LRTOMT, GATM-AS1, AZGP1, RAMP2-AS1, SPDYE5, which are preferably expressed or upregulated.

In certain embodiments of the invention, the tumor stem cell or progenitor cell further expresses or has an increased expression of one or more of G1/S signature genes or one or more G2/M signature genes. In certain embodiments of the invention, the tumor stem cell or progenitor cell further expresses or has an increased expression of one or more of MCM5, PCNA, TYMS, FEN1, MCM2, MCM4, RRM1, UNG, GINS2, MCM6, CDCA7, DTL, PRIM1, UHRF1, MLF1IP, HELLS, RFC2, RPA2, NASP, RAD51AP1, GMNN, WDR76, SLBP, CCNE2, UBR7, POLD3, MSH2, ATAD2, RAD51, RRM2, CDC45, CDC6, EXO1, TIPIN, DSCC1, BLM, CASP8AP2, USP1, CLSPN, POLA1, CHAF1B, BRIP1, E2F8, HMGB2, CDK1, NUSAP1, UBE2C, BIRC5, TPX2, TOP2A, NDC80, CKS2, NUF2, CKS1B, MKI67, TMPO, CENPF, TACC3, FAM64A, SMC4, CCNB2, CKAP2L, CKAP2, AURKB, BUB1, KIF11, ANP32E, TUBB4B, GTSE1, KIF20B, HJURP, HJURP, CDCA3, HN1, CDC20, TTK, CDC25C, KIF2C, RANGAP1, NCAPD2, DLGAP5, CDCA2, CDCA8, ECT2, KIF23, HMMR, AURKA, PSRC1, ANLN, LBR, CKAP5, CENPE, CTCF, NEK2, G2E3, GAS2L3, CBX5, CENPA.

In certain embodiments of the invention, the one or more astrocyte signature gene or polypeptide is selected from the group consisting of APOE, SPARCL1, SPOCK1, CRYAB, ALDOC, CLU, EZR, SORL1, MLC1, ABCA1, ATP1B2, PAPLN, CA12, BBOX1, RGMA, AGT, EEPD1, CST3, SSTR2, SOX9, RND3, EDNRB, GABRB1, PLTP, JUNB, DKK3, ID4, ADCYAP1R1, GLUL, EPAS1, PFKFB3, ANLN, HEPN1, CPE, RASL10A, SEMA6A, ZFP36L1, HEY1, PRLHR, TACR1, JUN, GADD45B, SLC1A3, CDC42EP4, MMD2, CPNE5, CPVL, RHOB, NTRK2, CBS, DOK5, TOB2, FOS, TRIL, NFKBIA, SLC1A2, MTHFD2, IER2, EFEMP1, ATP13A4, KCNIP2, ID1, TPCN1, LRRC8A, MT2A, FOSB, L1CAM, LIX1, HLA-E, PEA15, MT1X, 1L33, LPL, IGFBP7, C1orf61, FXYD7, TIMP3, RASSF4, HNMT, JUND, NHSL1, ZFP36L2, SRPX, DTNA, ARHGEF26, SPON1, TBC1D10A, DGKG, LHFP, FTH1, NOG, LCAT, LRIG1, GATSL3, EGLN3, ACSL6, HEPACAM, ST6GAL2, KIF21A, SCG3, METTL7A, CHST9, RFX4, P2RY1, ZFAND5, TSPAN12, SLC39A11, NDRG2, HSPB8, IL11RA, SERPINA3, LYPD1, KCNH7, ATF3, TMEM151B, PSAP, HIF1A, PON2, HIF3A, MAFB, SCG2, GRIA1, ZFP36, GRAMD3, PER1, TNS1, BTG2, CASQ1, GPR75, TSC22D4, NRP1, DNASE2, DAND5, SF3A1, PRRT2, DNAJB1, F3, which are preferably expressed or upregulated.

In certain embodiments of the invention, the one or more astrocyte signature gene or polypeptide is selected from the group consisting of APOE, SPARCL1, ALDOC, CLU, EZR, SORL1, MLC1, ABCA1, ATP1B2, RGMA, AGT, EEPD1, CST3, SOX9, EDNRB, GABRB1, PLTP, JUNB, DKK3, ID4, ADCYAP1R1, GLUL, PFKFB3, CPE, ZFP36L1, JUN, SLC1A3, CDC42EP4, NTRK2, CBS, DOK5, FOS, TRIL, SLC1A2, ATP13A4, ID1, TPCN1, FOSB, LIX1, IL33, TIMP3, NHSL1, ZFP36L2, DTNA, ARHGEF26, TBC1D10A, LHFP, NOG, LCAT, LRIG1, GATSL3, ACSL6, HEPACAM, SCG3, RFX4, NDRG2, HSPB8, ATF3, PON2, ZFP36, PER1, BTG2, NRP1, PRRT2, F3, which are preferably expressed or upregulated.

In certain embodiments of the invention, the one or more astrocyte signature gene or polypeptide is selected from the group consisting of SPOCK1, CRYAB, PAPLN, CA12, BBOX1, SSTR2, RND3, EPAS1, ANLN, HEPN1, RASL10A, SEMA6A, HEY1, PRLHR, TACR1, GADD45B, MMD2, CPNE5, CPVL, RHOB, TOB2, NFKBIA, MTHFD2, IER2, EFEMP1, KCNIP2, LRRC8A, MT2A, L1CAM, HLA-E, PEA15, MT1X, LPL, IGFBP7, C1orf61, FXYD7, RASSF4, HNMT, JUND, SRPX, SPON1, DGKG, FTH1, EGLN3, ST6GAL2, KIF21A, METTL7A, CHST9, P2RY1, ZFAND5, TSPAN12, SLC39A11, IL11RA, SERPINA3, LYPD1, KCNH7, TMEM151B, PSAP, HIF1A, HIF3A, MAFB, SCG2, GRIA1, GRAMD3, TNS1, CASQ1, GPR75, TSC22D4, DNASE2, DAND5, SF3A1, DNAJB1, which are preferably expressed or upregulated.

In certain embodiments of the invention, the one or more oligodendrocyte signature gene or polypeptide is selected from the group consisting of LMF1, OLIG1, SNX22, POLR2F, LPPR1, GPR17, DLL3, ANGPTL2, SOX8, RPS2, FERMT1, PHLDA1, RPS23, NEU4, SLC1A1, LIMA1, ATCAY, SERINC5, CDH13, CXADR, LHFPL3, ARL4A, SHD, RPL31, GAP43, IFITM10, SIRT2, OMG, RGMB, HIPK2, APOD, NPPA, EEF1B2, RPS17L, FXYD6, MYT1, RGR, OLIG2, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, RTKN, UQCRB, FA2H, MIF, TUBB3, COX7C, AMOTL2, THY1, NPM1, MARCKSL1, LIMS2, PHLDB1, RAB33A, GRIA2, OPCML, SHISA4, TMEFF2, ACAT2, HIP1, NME1, NXPH1, FDPS, MAP1A, DLL1, TAGLN3, PID1, KLRC2, AFAP1L2, LDHB, TUBB4A, ASIC1, TM7SF2, GRIA4, SGK1, P2RX7, WSCD1, ATP5E, ZDHHC9, MAML2, UGT8, C2orf27A, VIPR2, DHCR24, NME2, TCF12, MEST, CSPG4, GAS5, MAP2, LRRN1, GRIK2, FABP7, EIF3E, RPL13A, ZEB2, EIF3L, BIN1, FGFBP3, RAB2A, SNX1, KCNIP3, EBP, CRB1, RPS10-NUDT3, GPR37L1, CNP, DHCR7, MICAL1, TUBB, FAU, TMSB4X, PHACTR3, which are preferably expressed or upregulated.

In certain embodiments of the invention, the one or more oligodendrocyte signature gene or polypeptide is selected from the group consisting of OLIG1, SNX22, GPR17, DLL3, SOX8, NEU4, SLC1A1, LIMA1, ATCAY, SERINC5, LHFPL3, SIRT2, OMG, APOD, MYT1, OLIG2, RTKN, FA2H, MARCKSL1, LIMS2, PHLDB1, RAB33A, OPCML, SHISA4, TMEFF2, NME1, NXPH1, GRIA4, SGK1, ZDHHC9, CSPG4, LRRN1, BIN1, EBP, CNP, which are preferably expressed or upregulated.

In certain embodiments of the invention, the one or more oligodendrocyte signature gene or polypeptide is selected from the group consisting of LMF1, POLR2F, LPPR1, ANGPTL2, RPS2, FERMT1, PHLDA1, RPS23, CDH13, CXADR, ARLAA, SHD, RPL31, GAP43, IFITM10, RGMB, HIPK2, NPPA, EEF1B2, RPS17L, FXYD6, RGR, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, UQCRB, MIF, TUBB3, COX7C, AMOTL2, THY1, NPM1, GRIA2, ACAT2, HIP1, FDPS, MAP1A, DLL1, TAGLN3, PID1, KLRC2, AFAP1L2, LDHB, TUBB4A, ASIC1, TM7SF2, P2RX7, WSCD1, ATP5E, MAML2, UGT8, C2orf27A, VIPR2, DHCR24, NME2, TCF12, MEST, GAS5, MAP2, GRIK2, FABP7, EIF3E, RPL13A, ZEB2, EIF3L, FGFBP3, RAB2A, SNX1, KCNIP3, CRB1, RPS10-NUDT3, GPR37L1, DHCR7, MICAL1, TUBB, FAU, TMSB4X, PHACTR3, which are preferably expressed or upregulated.

In certain embodiments of the invention, the tumor astrocyte does not express or has a reduced expression of one or more of LMF1, OLIG1, SNX22, POLR2F, LPPR1, GPR17, DLL3, ANGPTL2, SOX8, RPS2, FERMT1, PHLDA1, RPS23, NEU4, SLC1A1, LIMA1, ATCAY, SERINC5, CDH13, CXADR, LHFPL3, ARL4A, SHD, RPL31, GAP43, IFITM10, SIRT2, OMG, RGMB, HIPK2, APOD, NPPA, EEF1B2, RPS17L, FXYD6, MYT1, RGR, OLIG2, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, RTKN, UQCRB, FA2H, MIF, TUBB3, COX7C, AMOTL2, THY1, NPM1, MARCKSL1, LIMS2, PHLDB1, RAB33A, GRIA2, OPCML, SHISA4, TMEFF2, ACAT2, HIP1, NME1, NXPH1, FDPS, MAP1A, DLL1, TAGLN3, PID1, KLRC2, AFAP1L2, LDHB, TUBB4A, ASIC1, TM7SF2, GRIA4, SGK1, P2RX7, WSCD1, ATP5E, ZDHHC9, MAML2, UGT8, C2orf27A, VIPR2, DHCR24, NME2, TCF12, MEST, CSPG4, GAS5, MAP2, LRRN1, GRIK2, FABP7, EIF3E, RPL13A, ZEB2, EIF3L, BIN1, FGFBP3, RAB2A, SNX1, KCNIP3, EBP, CRB1, RPS10-NUDT3, GPR37L1, CNP, DHCR7, MICAL1, TUBB, FAU, TMSB4X, PHACTR3.

In certain embodiments of the invention, the tumor astrocyte does not express or has a reduced expression of one or more of OLIG1, SNX22, GPR17, DLL3, SOX8, NEU4, SLC1A1, LIMA1, ATCAY, SERINC5, LHFPL3, SIRT2, OMG, APOD, MYT1, OLIG2, RTKN, FA2H, MARCKSL1, LIMS2, PHLDB1, RAB33A, OPCML, SHISA4, TMEFF2, NME1, NXPH1, GRIA4, SGK1, ZDHHC9, CSPG4, LRRN1, BIN1, EBP, CNP.

In certain embodiments of the invention, the tumor astrocyte does not express or has a reduced expression of one or more of LMF1, POLR2F, LPPR1, ANGPTL2, RPS2, FERMT1, PHLDA1, RPS23, CDH13, CXADR, ARL4A, SHD, RPL31, GAP43, IFITM10, RGMB, HIPK2, NPPA, EEF1B2, RPS17L, FXYD6, RGR, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, UQCRB, MIF, TUBB3, COX7C, AMOTL2, THY1, NPM1, GRIA2, ACAT2, HIP1, FDPS, MAP1A, DLL1, TAGLN3, PID1, KLRC2, AFAP1L2, LDHB, TUBB4A, ASIC1, TM7SF2, P2RX7, WSCD1, ATP5E, MAML2, UGT8, C2orf27A, VIPR2, DHCR24, NME2, TCF12, MEST, GAS5, MAP2, GRIK2, FABP7, EIF3E, RPL13A, ZEB2, EIF3L, FGFBP3, RAB2A, SNX1, KCNIP3, CRB1, RPS10-NUDT3, GPR37L1, DHCR7, MICAL1, TUBB, FAU, TMSB4X, PHACTR3.

In certain embodiments of the invention, the tumor oligodendrocyte does not express or has a reduced expression of one or more of APOE, SPARCL1, SPOCK1, CRYAB, ALDOC, CLU, EZR, SORL1, MLC1, ABCA1, ATP1B2, PAPLN, CA12, BBOX1, RGMA, AGT, EEPD1, CST3, SSTR2, SOX9, RND3, EDNRB, GABRB1, PLTP, JUNB, DKK3, ID4, ADCYAP1R1, GLUL, EPAS1, PFKFB3, ANLN, HEPN1, CPE, RASL10A, SEMA6A, ZFP36L1, HEY1, PRLHR, TACR1, JUN, GADD45B, SLC1A3, CDC42EP4, MMD2, CPNE5, CPVL, RHOB, NTRK2, CBS, DOK5, TOB2, FOS, TRIL, NFKBIA, SLC1A2, MTHFD2, IER2, EFEMP1, ATP13A4, KCNIP2, ID1, TPCN1, LRRC8A, MT2A, FOSB, L1CAM, LIX1, HLA-E, PEA15, MT1X, IL33, LPL, IGFBP7, C1orf61, FXYD7, TIMP3, RASSF4, HNMT, JUND, NHSL1, ZFP36L2, SRPX, DTNA, ARHGEF26, SPON1, TBC1D10A, DGKG, LHFP, FTH1, NOG, LCAT, LRIG1, GATSL3, EGLN3, ACSL6, HEPACAM, ST6GAL2, KIF21A, SCG3, METTL7A, CHST9, RFX4, P2RY1, ZFAND5, TSPAN12, SLC39A11. NDRG2, HSPB8, IL11RA, SERPINA3, LYPD1, KCNH7, ATF3, TMEM151B, PSAP, HIF1A, PON2, HIF3A, MAFB, SCG2, GRIA1, ZFP36, GRAMD3, PER1, TNS1, BTG2, CASQ1, GPR75, TSC22D4, NRP1, DNASE2, DAND5. SF3A1, PRRT2, DNAJB1, F3.

In certain embodiments of the invention, the tumor oligodendrocyte does not express or has a reduced expression (e.g. in CIC mutant cells compared to CIC wild type cells) of one or more of APOE, SPARCL1, ALDOC, CLU, EZR, SORL1, MLC1, ABCA1, ATP1B2, RGMA, AGT, EEPD1, CST3, SOX9, EDNRB, GABRB1, PLTP, JUNB, DKK3, ID4, ADCYAP1R1, GLUL, PFKFB3, CPE, ZFP36L1, JUN, SLC1A3, CDC42EP4, NTRK2, CBS, DOK5, FOS, TRIL, SLC1A2, ATP13A4, ID1, TPCN1, FOSB, LIX1, IL33, TIMP3, NHSL1, ZFP36L2, DTNA, ARHGEF26, TBC1D10A, LHFP, NOG, LCAT, LRIG1, GATSL3, ACSL6, HEPACAM, SCG3, RFX4, NDRG2, HSPB8, ATF3, PON2, ZFP36, PER1, BTG2, NRP1, PRRT2, F3.

In certain embodiments of the invention, the tumor oligodendrocyte does not express or has a reduced expression (e.g. in CIC mutant cells compared to CIC wild type cells) of one or more of SPOCK1, CRYAB, PAPLN, CA12, BBOX1, SSTR2, RND3, EPAS1, ANLN, HEPN1, RASL10A, SEMA6A, HEY1, PRLHR, TACR1, GADD45B, MMD2, CPNE5, CPVL, RHOB, TOB2, NFKBIA, MTHFD2, IER2, EFEMP1, KCNIP2, LRRC8A, MT2A, L1CAM, HLA-E, PEA15, MT1X, LPL, IGFBP7, C1orf61, FXYD7, RASSF4, HNMT, JUND, SRPX, SPON1, DGKG, FTH1, EGLN3, ST6GAL2, KIF21A, METTL7A, CHST9, P2RY1, ZFAND5, TSPAN12, SLC39A11, IL11RA, SERPINA3, LYPD1, KCNH7, TMEM151B, PSAP, HIF1A, HIF3A, MAFB, SCG2, GRIA1, GRAMD3, TNS1, CASQ1, GPR75, TSC22D4, DNASE2, DAND5, SF3A1, DNAJB1.

In certain embodiments, the tumor stem/progenitor cell, astrocyte, and/or oligodendrocyte as referred to herein expresses or has an increased expression of one or more of ALG9, AP3S1, ARRDC3, BRAT1, CLN3, CNTNAP2, COL16A1, CTTN, DLD, DOCK10, DSEL, ECI2, EP300, ETV1, ETV5, FAR1, FOXRED1, FYTTD1, GATS, GFRA1, GLT25D2, GPR56, IGSF8, KANK1, KIAA1467, KIF22, LNX1, LPCAT1, ME3, MEGF11, MRPS16, NAV1, NFIA, NIN, NLGN3, NUP188, PCDH15, PCDHB9, PPP2R2B, PPWD1, PTN, RASD1, RNF214, SDC3, SEC24B, SLC38A10, STIM1, TMEM181, TTLL5, VARS, YJEFN3, ZNF451, ZNF564.

In certain embodiments, the tumor stem/progenitor cell, astrocyte, and/or oligodendrocyte as referred to herein does not express or has an decreased expression of one or more of ANKMY2, ATF4, BRK1, BTF3L4, EIF3C, EVI2A, GFAP, MAD2L2, MPV7, MRPL46, NDUFV1, NFE2L2, RAB1A, RCOR3, RSL1D1, TTC14.

In an aspect, the invention relates to an (isolated) cell characterized by comprising the expression of one or more a signature genes or polypeptide or combinations of signature genes/proteins as defined herein.

In a further aspect, the invention relates to a glioma gene expression signature characterized by one or more signature gene or polypeptide or combinations of signature genes/proteins as defined herein.

In another aspect, the invention provides a method of diagnosing, prognosing, and/or staging a melanoma, as well as predicting and monitoring a treatment response, comprising detecting a first level of expression, activity and/or function of one or more signature genes or one or more products of one or more signature genes in one or more cell(s) of the melanoma and comparing the detected level to a control of level of signature gene or gene product expression, activity and/or function, wherein a difference in the detected level and the control level indicates a malignant, microenvironmental, or immunologic state of the melanoma.

In certain embodiments, the melanoma is a metastatic melanoma. In certain embodiments, the melanoma is a recurrent melanoma. By recurrent melanoma is meant a melanoma that has been treated to the extent that it had become undetectable, but reappears subsequent to the treatments. The time to recurrence can be, e.g., six months, a year, two years, three years, five years, or longer.

In certain embodiments of the invention, the melanoma tumor, tissue, or cell comprises a BRAF mutation. In certain embodiments of the invention, the melanoma tumor, tissue, or cell comprises an NRAS mutation. In certain embodiments, the melanoma tumor, tissue, or cell is from a patient who progressed through chemotherapy, including but not limited to treatment with vemurafenib or a combination of vemurafenib and trametinib.

In certain embodiments, the one or more signature gene(s) or gene network comprises a MITF-high associated gene. In certain embodiments, the signature gene(s) or gene network comprises an AXL-high associated gene. In certain embodiments, MITF-high associated genes include TYR, PMEL and MLANA. In certain embodiments, AXL associated genes include AXL and NGFR.

In certain embodiments, the expression state of the one or more signature gene(s) or gene network indicates the functional state of an immune cell or response in the tumor. In one such embodiment, the expression state of the one or more signature gene(s) or gene network indicates the functional state of a T cell from the melanoma. In another such embodiment, the expression state of the one or more signature gene(s) or gene network indicates the functional state of a B cell from the melanoma. In one such embodiment, the expression state of the one or more signature gene(s) or gene network indicates the functional state of a CD4+ T cell from the melanoma. In one such embodiment, the expression state of the one or more signature gene(s) or gene network indicates the functional state of a CD8+ T cell from the melanoma. In another such embodiment, the expression state of the one or more signature gene(s) or gene network indicates the functional state of a macrophage from the melanoma. In yet another such embodiment, the expression state of the one or more signature gene(s) or gene network is an indicator of immune cell cytotoxicity, exhaustion or a naïve marker. In another such embodiment, the expression state of the one or more signature gene(s) or gene network is an indicator of the status of an immune checkpoint.

In certain embodiments, the expression state of the one or more signature gene(s) or gene network indicates an aspect of the cell cycle of a cell of the tumor. In one such embodiment, the expression state indicates whether a cell of the tumor is low-cycling or high-cycling. In another such embodiment, the one or more signature gene(s) is a cell cycle regulator, for example, including but not limited to a cyclin or a cyclin-dependent kinase. The one or more signature genes may be cyclin D3 (CCND3) or KDM5B (JAR1D1B), wherein CCND3 indicates high-cycling tumors and KDM5B indicates non-cycling cells. The tumor may be melanoma or glioma. KDM5B is uniquely expressed in quiescent cells, so targeting it is important in both melanoma or glioma. CCND3 is uniquely expressed in proliferating cells in those melanomas that have a lot of proliferation. In one embodiment, CCND3 is a target directly or through CDK4 or 6 inhibition.

In certain embodiments, the expression state of the one or more signature gene(s) or gene network is an indicator of drug resistance.

In an embodiment of the invention, the level or expression of one or more signature gene(s) or gene network is determined by measuring the level or expression of a nucleic acid. In one such embodiment, the level or expression of a signature gene is measured by single-cell RNA sequencing. In one embodiment of the invention, the level or expression of one or more signature gene(s) or gene network is determined by measuring the level or expression of the protein encoded by the gene(s) or gene network. In one embodiment of the invention, the level or expression of the protein encoded one or more signature gene(s) or gene network is determined by, e.g., absorbance assays and colorimetric assays such as those known in the art.

In certain embodiments, the level or expression of one or more signature gene(s) is determined by measuring expression in single cells. In other embodiments the level or expression of one or more signature gene(s) is measured in a melanoma tumor or tissue expression of signature genes determined by deconvolution of the bulk expression properties of the tumor. In other embodiments, the signature genes are detected by immunofluorescence or by mass cytometry (CyTOF) or by in situ hybridization.

The invention further provides a method for monitoring a subject undergoing a treatment or therapy for a melanoma comprising detecting a level of expression, activity and/or function of one or more signature genes or one or more products of one or more signature genes of the melanoma in the absence of the treatment or therapy and comparing the level of expression, activity and/or function of one or more signature genes or one or more products of one or more signature genes in the presence of the treatment or therapy, wherein a difference in the level of expression, activity and/or function of one or more signature genes or one or more products of one or more signature genes in the presence of the treatment or therapy indicates whether the patient is responsive to the treatment or therapy.

In another aspect, the present invention provides for a method of treating melanoma or enhancing treatment of a melanoma, which comprises administering an agent that increases the function of one or more signature genes or one or more products of one or more signature genes in one or more cell(s) of the melanoma, wherein the one or more signature genes or one or more products of one or more signature genes comprises a signature gene of Table 15, Table 12, Table 13 or Table 14. The one or more signature genes may be CXCL12 or CCL19. The one or more signature genes may be PDCD1, TIGIT, HAVCR2, SIT1, LAG3, CTLA4, FAM3C, TNFRSF9, SYT11, GUSBP3. SIRPG, LY6E, CCL13, SUMO2, IL2RG, CD74, CBLB, FOXN3, SLA, FKBP1A, CD27, SP100, IK, CCL3, CXCL13, TNFRSF1B, RGS2, RNF19A, INPP5F, XCL2, HLA-DMA, UQCRC1, WARS, EIF3L, KCNK5, TMBIM6, CD200, ZC3H7A, SH2D1A, ATP1B3, MYO7A, THADA, PARK7, EGR2, FDFT1, CRTAM, IFII6, LAG3, NFATC1, TIM3, PD-1, BTLA or CBLB. The one or more signature genes may be C1S, C1R, C3, C4A, CFB, C1QA, C1QB or C1QC.

In another aspect, the present invention provides for a method of treating melanoma or enhancing treatment of a melanoma, which comprises administering an agent that modulates the activity and/or expression of one or more signature genes or one or more products of one or more signature genes in one or more cell(s) of the melanoma, wherein the one or more signature genes or one or more products of one or more signature genes is a complement system gene or gene product. The agent may modulate the activity and/or expression of C1S, C1R, C3, C4A, CFB, C1QA, C1QB, C1QC, C5 or SERPING1. The agent may be a CRISPR-Cas system that activates expression of a complement system gene. The agent may target a complement defense gene selected from the group consisting of CD46, CD55, and CD59. The agent may be a CRISPR-Cas system that targets the complement defense gene, whereby the gene is knocked out or expression is decreased. The agent may be a natural product, whereby the complement system is activated in a tumor.

In another aspect, the present invention provides for a method of identifying at least one tumor specific T Cell receptor (TCR) for use in adoptive cell transfer, said method comprising: identifying by sequencing, TCRs from single tumor infiltrating T cells obtained from a tumor sample; selecting the TCRs that are clonal and/or are derived from a T cell that expresses one or more signature genes of exhaustion; and cloning the selected TCRs into a non-naturally occurring vector. The one or more signature genes of exhaustion may be PDCD1, TIGIT, HAVCR2, SIT1, LAG3, CTLA4, FAM3C, TNFRSF9, SYT11, GUSBP3, SIRPG, LY6E, CXCL13, SUMO2, IL2RG, CD74, CBLB, FOXN3, SLA, FKBP1A, CD27, SP100, IK, CCL3, CXCL13, TNFRSF1B, RGS2, RNF19A, INPP5F, XCL2, HLA-DMA, UQCRC1, WARS, EIF3L, KCNK55 TMBIM6, CD200, ZC3H7A, SH2D1A, ATP1B3, MYO7A, THADA, PARK7, EGR2, FDFT1, CRTAM, IFI16, LAG3, NFATC1, TIM3, PD-1, BTLA or CBLB.

In another aspect, the present invention provides for a method of treating a subject in need thereof suffering from cancer comprising administering at least one activated T cell to the subject expressing at least one TCR pair identified by a method described herein. In another aspect, the present invention provides for a non-naturally occurring T cell expressing a tumor specific TCR pair identified by the method a method described herein.

In another aspect, the present invention provides for a personalized cancer treatment for a patient in need thereof comprising: determining clonality of TCRs in tumor infiltrating T cells from the patient, and/or detecting expression of one or more signature genes for exhaustion, and/or detecting expression of one or more signature genes correlated to T cell abundance; and administering an agent that stimulates the patients preexisting immune response if (i) at least one clonal TCR is determined and/or (ii) one or more signature genes for exhaustion is detected and/or (iii) one or more signature genes correlated to T cell abundance is detected. The agent may be a checkpoint inhibitor.

In certain embodiments, the gene signatures described herein encode surface exposed or transmembrane proteins, such that they can be targeted by CAR T cells, therapeutic antibodies or fragments thereof or antibody drug conjugates or fragments thereof.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention. Nothing herein is intended as a promise.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1A-1D depicts tumor dissection to single cells and analyses by single-cell RNA-seq. Panel (A) depicts the steps of tumor analysis from resection to flow-cytometry, single-cell RNA-sequencing and downstream analysis. Panel (B): Chromosomal landscape of inferred large-scale copy number variations (CNVs) distinguishes malignant from non-malignant cells. One example tumor (Mel80) is shown with individual cells (yaxis) and chromosomal regions (x-axis). Amplifications (red) or deletions (blue) were inferred by averaging expression over 100-gene stretches on the respective chromosomes. Inferred CNVs are strongly concordant with calls from whole-exome sequencing (WES, bottom). Panels (C,D) Single cell expression profiles distinguish malignant and non-malignant cell types. Shown are t-SNE (t-Distributed Stochastic Neighbor Embedding) plots of malignant (C, shown are the six tumors each with >50 malignant cells) and non-malignant (D) cells (as called from inferred CNVs as in B) from 11 tumors with >100 cells per tumor (color code). Clusters of non-malignant cells (called by DBScan, Methods) are marked by dashed ellipses and were annotated as T cells, B cells, macrophages, CAFs and endothelial cells, based on preferentially expressed genes (FIG. 7 and Table 2-3). This analysis separates multiple non-tumor cell types, such as T cells, B cells, macrophages, Tumor Associated Fibroblasts (TAFs, also called Cancer Associated Fibroblasts or CAFs) and endothelial cells.

FIG. 2A-2D depicts that single-cell RNA-seq distinguishes cell cycle and other states among malignant cells. (A) Estimation of the cell cycle state of individual malignant cells (circles) based on relative expression of G1/S (x-axis) and G2/M (y-axis) gene-sets in a low-cycling (Mel79, top) and a high-cycling (Mel78, bottom) tumor. Cells are colored by their inferred cell cycle states, with cycling cells (red), intermediate (bright red) and non-cycling cells (black); cells with high expression of KDM5B (Z-score>2) are marked in cyan filling. (B) IHC staining (40× magnification) for Ki67+ cells shows a high concordance with the signature-based frequency of cycling cells for Mel79 and Mel78 (as for other tumors; FIG. S4C). (C) KDM5B/Ki67 staining (40× magnification) in corresponding tissue showing small clusters of KDM5B-high expressing cells that are all negative for Ki67 (see also FIG. 9). (D) An expression program specific to Region 1 of Mel79, based on multifocal sampling. The relative expression of genes (rows) is shown for cells (columns) ordered by the average expression of the entire gene-set. The region-of-origin of each cell is indicated in the top panel (see also FIG. 10).

FIG. 3A-3F depicts MITF- and AXL-associated expression programs and their variation among tumors, within tumors, and following treatment. Panel (A) depicts average expression signatures for the AXL program (y-axis) or the MITF program (x-axis) stratify tumors into ‘MITF-high’ (black) or ‘AXL-high’ (red). (B) Single-cell profiles show a negative correlation between the AXL program (y-axis) and MITF program (x-axis) across individual malignant cells within the same tumor; cells are colored by the relative expression of the MITF (black) and AXL (red) programs. Cells in both states are found in all examined tumors, including three tumors (Mel79, Mel80 and Mel81) without prior systemic treatment, indicating that dormant resistant (AXL-high) cells may already be present in treatment naïve patients. (C) Mel81 and Mel80 immunofluorescence staining of MITF (green nuclei) and AXL (red), validating the mutual exclusivity among individual cells within the same tumor (see also FIG. 15). (D) Relative expression (centered) of the AXL-program (top) and MITF-program (bottom) genes in six matched pre-treatment (white boxes) and post-relapse (gray boxes) samples from patients who progressed through RAF/MEK inhibition therapy; numbers at the top indicate patient index. Samples are sorted by the average relative expression of the AXL vs. MITF gene-sets. In all cases, the relapsed samples had increased ratio of AXL/MITF expression compared to their pre-treatment counterpart. This consistent shift of all six patients is statistically significant (P<0.05, binomial test), as are the individual increases in AXL/MITF for four of the six sample pairs (P<0.05, t-test; black and gray arrows denote increases that are individually significant or non-significant, respectively). (E) Flow-cytometric quantification of the relative fraction of cells with AXL-high (log-scale, y-axis) expression, when cells were treated with increasing doses of RAF/MEK-inhibition (dabrafenib and trametinib in a 10:1 ratio at indicated doses). In all examined cell lines (x-axis), there was a dose-dependent increase in the AXL-high expressing cell fraction. (F) Quantitative, multiplexed single-cell immunofluorescence for AXL expression (y-axis top), MAP-kinase pathway inhibition (pERK levels, y-axis) and viability (y-axis bottom) in the example cell line WM88 treated with increasing concentrations (y-axis) of either RAF inhibitor alone (black bars) or a combination of RAF/MEK-inhibitors (yellow bars). Applicants observe increasing relative AXL-high expressing cell fraction (top panel), consistent with flow-cytometry, as well as a dosedependent decrease of p-ERK (middle) and viability (bottom), overall consistent with phenotypic selection (killing of MITF-high cells) as part of the shift towards the AXL-high fraction (see FIG. 18-19 for additional cell lines).

FIG. 4A-4G shows deconvolution of bulk melanoma profiles by specific signatures of non-cancer cell types revealing cell-cell interactions. Panel (A) Bulk tumors segregate to distinct clusters based on their inferred cell type composition. Top panel: heat map showing the relative expression of gene sets defined from single-cell RNA-seq as specific to each of five cell types from the tumor microenvironment (y-axis) across 495 melanoma TCGA bulk-RNA signatures (x-axis). Each column is one tumor and tumors are partitioned into 10 distinct patterns identified by K-means clustering (vertical lines and cluster numbers at the top). Lower panels show from top to bottom tumor purity, specimen location (from TCGA), and AXL/MITF scores. Tumor purity as estimated by the expression of cell-type specific gene-sets (“RNA”) was strongly correlated with that estimated by ABSOLUTE mutation analysis (“DNA”, R=0.8, bottom panel, both smoothed with a moving average of 40 tumors). Tumor classification, and in particular tumors with high abundance of CAFs, is strongly correlated with an increased ratio of AXLprogram/MITF-program expression (bottom). (B) Inferred cell-to-cell interactions between CAFs and T cells. Scatter plot compares for each gene (circle) the correlation of its expression with inferred T cell abundance across bulk tumors (y-axis, from TCGA transcriptomes) to how specific its expression is to CAFs vs. T cells (x-axis, based on single-cell transcriptomes). Genes that are highly specific to CAFs in a single cell analysis of tumors (red), but also associated with high T cell abundance in bulk tumors (black border) are key candidates for CAF/T cell interactions. This analysis identified known (CXCL12, CCL19) genes linked to immune cell chemotaxis and putative immune modulators, including multiple complement factors (C1R. C1S, C3, C4A, CFB and C1NH [SERPING1]). (C) Correlation between quantitative immunofluorescence signal (% Area) of C3 and CD8 levels across 308 core biopsies of melanoma tissue microarrays. Shown are 90 included samples with 80 tumor specimens (black dots) showing a correlation (R=0.86) between C3/C8 signal and 10 normal control specimens (grey dots). See FIG. 27A-F for normalization and additional specimens. (D) Correlation coefficient (y-axis) between the average expression of CAF-derived complement factors shown in (B) and that of T cell markers (CD3/D/E/G, CD8A/B) across 26 TCGA cancer types with >100 samples (x-axis, left panel) and across 36 GTEx tissue types with >100 samples (x axis, right panel). Bars are colored based on correlation ranges as indicated at the bottom. Panel (E) shows correlations between the inferred frequencies of distinct cell types across TCGA samples. Panel (F) depicts correlated abundance of CD3+ cells and alpha-SMA+ TAFs by IHC. Panel (G) provides Kaplan Meier plots for progression free survival of patients included in the melanoma TCGA study, demonstrating that stratification by the frequency of TAFs (left) or MITF-levels (right) are associated with significant survival outcomes only in the context of low-immune melanomas.

FIG. 5A-5K shows a T-cell analysis that distinguishes activation-dependent and independent variation in coexpressed exhaustion markers. Panel (A) shows stratification of T cells into CD4+ and CD8+ cells (upper panel), CD25+FOXP3+ and other CD4 cells (middle panel) and their associated inferred activation state (lower panel, based on average expression of the cytotoxic and naïve gene-sets shown in (B)). (B) Average expression of markers of cytotoxicity, exhaustion and naïve cell states (rows) in (left to right) Tregs, CD4+ T cells, and CD8+ T cells; CD4+ and CD8+ T cells are each further divided into five bins by their cytotoxic score (ratio of cytotoxic to naïve marker expression levels), showing an activationdependent co-expression of exhaustion markers. Bottom: proportion of cycling cells (calculated as in FIG. 2B). Asterisks denote significant enrichment or depletion of cycling cells in a specific subset compared to the corresponding set of CD4+ or CD8+ T cells (P<0.05, hypergeometric test). (C) Immunofluorescence of PD-1 (upper panel, green), TIM-3 (middle panel, red) and their overlay (lower panel) validates their co-expression. (D) Activation-independent variation in exhaustion states within highly cytotoxic T cells. Scatter plot shows the cytotoxic score (x-axis) and exhaustion score (y-axis, average expression of the Mel75 exhaustion program shown in FIG. 31) of each CD8+ T cell from Mel75. In addition to the overall correlation between cytotoxicity and exhaustion, the cytotoxic cells can be sub-divided into highly exhausted (red) and lowly exhausted cells (green) based on comparison to a LOWESS regression (black line). (E-F) Relative expression (log 2 fold-change) in high vs. low exhaustion cytotoxic CD8+ T cells from five tumors (x-axis), including 28 genes that were significantly induced (P<0.05, permutation test) in high-exhaustion cells across tumors (E) and 272 genes that were variably expressed across tumors (F). Three independently derived exhaustion gene-sets were used to define high and low exhaustion cells (Mel75, (45, 49), see Methods), and the corresponding results are represented as distinct columns for each tumor. (G) Expanded TcR clones. Cells were assigned to clusters of TCR segment usage (black bars; FIG. 33), and cluster size (x-axis) was evaluated for significance by control analysis in which TCR segments were shuffled across cells (grey bars). The percentage of Mel75 cells (y-axis) is shown for clusters of small size (1-4 cells) that likely represent non-expanded cells, medium size (5-6 cells) that may reflect expanded clones (FDR=0.12), and large size that most likely reflect expanded clones (FDR=0.005). (H) Expanded clones are depleted of nonexhausted cells and enriched for exhausted cells. Mel75 cells were divided by exhaustion score into low exhaustion (green, bottom 25% of cells) and medium-to-high exhaustion (red, top 75%). Shown is the relative frequency of these exhaustion subsets (y-axis) in each TCR-cluster group (x-axis, as defined in G), defined as log 2-ratio of the frequency in that group compared to the frequency across all Mel75 cells. All values were highly significant (P<10-5, binomial test). Panel (1) shows T-cells with cytotoxic activity (x-axis) sub-divided into highly exhausted (red) and lowly exhausted cells (green) based on the average levels of five exhaustion markers (PD1, TIGIT, TIM-3, LAG-3 and CTLA-4). Panels (J-K) show relative expression (log 2 fold-change) in high vs. low exhaustion cytotoxic CD8+ T-cells from three tumors (x-axis), including 10 genes that were significantly enriched (P<0.05, t-test) in high-exhaustion cells of at least two tumors (J) and 143 genes that were significantly enriched in high-exhaustion cells of only one tumors (K).

FIG. 6A-6B depicts classification of cells to malignant and non-malignant based on inferred CNV patterns. (A) Same as shown in FIG. 1B for another melanoma tumor (Mel78). (B) Each plot compares two CNV parameters for all cells in a given tumor: (1) CNV score (X-axis) reflects the overall CNV signal, defined as the mean square of the CNV estimates across all genomic locations; (2) CNV correlation (Y-axis) is the Pearson correlation coefficient between each cell's CNV pattern and the average CNV pattern of the top 5% of cells from the same tumor with respect to CNV signal (i.e., the most confidently-assigned malignant cells). These two values were used to classify cells as malignant (red; CNV score >0.04; correlation score >0.4; grey lines mark thresholds on plot), non-malignant (blue; CNV score <0.04; correlation score <0.4), or unresolved intermediates (black, all remaining cells). In four tumors (Mel58, 67, 72 and 74), Applicants sequenced primarily the immune infiltrates (CD45− cells) and there were only zero or one malignant cells by this definition; in those cases, CNV correlation is not indicative of malignant cells (since the top 5° % cells by CNV signal are primarily non-malignant) and therefore all cells except for one in Mel58 were defined as non-malignant. Note that while these thresholds are somewhat arbitrary, this classification was highly consistent with the clustering patterns of these cells (as shown in FIG. 1C) into clusters of malignant and non-malignant cells.

FIG. 7A-7I depicts identification of non-malignant cell types by tSNE clusters that preferentially express cell type markers. (A-H) Each plot shows the average expression of a set of known marker genes for a particular cell type (as indicated at the top) overlaid on the tSNE plot of non-malignant cells, as shown in FIG. 1C. Gray indicates cells with no or minimal expression of the marker genes (E, average log 2(TPM+1), below 4), dark red indicates intermediate expression (4<E<6), and light red indicates cells with high expression (E>6). (I) DBscan clusters derived from tSNE coordinates, with parameters eps=6 and min-points=10. Eleven clusters are indicated by numbers and colors.

FIG. 8A-8B depicts the limited influence of tumor site on RNA-seq patterns. (A-B) Heat maps show correlations of global expression profiles between tumors, which were ordered by metastatic site. Expression levels were first averaged over melanoma (A) or T cells (B) in each tumor and then centered across the different tumors before calculating Pearson correlation coefficients. Differential expression analysis conducted between the two groups of tumors found zero differentially expressed genes with FDR of 0.05 based on a shuffling test for both T cells and melanoma cells.

FIG. 9A-9E shows the identification and characterization of cycling malignant cells. (A) Heat map showing relative expression of G1/S (top) and G2/M (bottom) genes (rows, as defined from integration of multiple datasets; Methods) across cycling cells (left panel, columns, ordered by the ratio of expression of G1/S genes to G2/M genes) and across all cells (right panel, columns, cycling cells ordered as in left panel followed by non-cycling cells at random order). Cycling cells were defined as those with significantly high expression of G1/S and/or G2/M genes (FDR<0.05 by t-test, and fold-change >4 compared to all malignant cells). (B) The frequency of inferred cycling cells (Y axis) in seven tumors (X axis) with >50 malignant cells/tumors, denoting low (<3%) or high (>20%) proliferation tumors. (C, upper panel) Significant correlation (P<0.038) between inferred proportion of cycling cells by single-cell transcriptome analysis (horizontal axis) and Ki67+ immunohistochemistry (IHC) (lower panel) of corresponding tumor slides (vertical axis). (D) Comparison of cycling cell expression programs between low- and high-proliferation tumors. Scatter plots compared the expression log-ratio between cycling and non-cycling cells in high-proliferation (y-axis) and low-proliferation (x-axis) tumors. Genes significantly upregulated (P<0.01, fold-change >2) in cycling cells in both types of tumor are marked in red. CCND3 (arrow) is significantly upregulated in cycling cells in high-proliferation tumors and downregulated in cycling cells in low-proliferation tumors. (E) Dual KDM5B (JAR1D1B)/Ki67 immunofluorescence staining of tissue slide of Mel80 (40× magnification). Consistent with findings presented for Mel78 and Mel79 in FIG. 2C, KDM5B-expressing cells (green nuclear staining) occurred in small clusters of two or more cells and do not express Ki67 (red nuclear staining), indicating that these cells are not undergoing cell division.

FIG. 10A-10B depicts immunohistochemistry of melanoma 79 shows gross differences between tumor parts and increased NF-κB levels in Region 1. (A) Tumor dissection into five regions. Left: melanoma tumor prior to dissection. Macroscopically distinct regions are highlighted by colored ovals. Right: The tumor was dissected into five pieces, which were further processed as individual samples. Regions 1, 3, 4 and 5 were included in the single-cell RNA-seq analysis, Cells from Region 2 were lost during library construction. (B) Corresponding histopathological cross-section of the tumor demonstrates distinct features of Region 1 compared to the other regions. Consistent with enrichment of cells in Region 1 expressing multiple markers that are highlighted in FIG. 2D, immunohistochemistry staining revealed increased staining of NF-κB and JunB in Region 1 (right lower panel, 40× magnification), compared to region Region 3 (right upper panel, 40×magnification).

FIG. 11A-11B depicts spatial heterogeneity in the expression of CD8+ T-cells. As shown in FIG. 2D for malignant cells, Applicants examined the expression differences between regions of Mel79 for other cell types. The only cell type for which Applicants had >10 cells in each of the regions was CD8+ T cells. Applicants thus focused on the differences among CD8+ T cells and found 62 genes that were preferentially expressed in region 1 (fold-change >2, FDR<0.05) and that partially overlapped the region 1-specific genes among the malignant cells (see Table 6). (A) Region 1-specific expression program of CD8+ T-cells (as shown in FIG. 2D for malignant cells). Bottom: heat map shows the relative expression of the 62 genes preferentially expressed in region 1, in all CD8+ T-cells from Mel79, ranked by their average expression of these genes. A subset of genes of interest are noted at the right. Top: assignment of cells to the four regions of Mel79. (B) Comparison of region 1 preferential expression between malignant cells (X-axis) and CD8+ T-cells (Y-axis). For each cell type, the scatterplot shows the log 2-ratio between the average expression of all cells in region 1 and those in all other regions.

FIG. 12 depicts intra-tumor heterogeneity in AXL and MITF programs. AXL-program (Y-axis) and MITF-program (X-axis) scores for malignant cells in each of the three tumors with a sufficient number of malignant cells (n>50) that were not included in FIG. 3B. Cells are colored from black to red by the relative AXL and MITF scores. The Pearson correlation coefficient is denoted on top.

FIG. 13A-13G depicts intra-tumor heterogeneity in MAPK signaling. Panel A shows average correlation among the MAPK signature genes within each of the tumors tumor cells and in control gene-sets (cont). As a control Applicants examined the average correlation of a 1000 randomly selected gene-sets with the same size and a similar distribution of average expression levels. The average correlation of the control gene-sets and their standard deviation are shown. Tumors are sorted by their correlation and five tumors (melanoma 80, 71, 78, 88 and 81) had a significantly high correlation (P<0.05, defined as having higher correlation than 95% of the control gene-sets). Panel B shows the correlation between the average of MAPK signature genes and the MITF score across cells in each of the tumors and in the control gene-sets. Three tumors (melanoma 80, 71 and 88) had a significant correlation (P<0.05, defined as having higher correlation than 95% of the control gene-sets) and these are the only three NRAS mutant tumors in this study, suggesting a connection between MAPK signaling and MITF activity within NRAS mutant tumors. Panels C-G depicts cells sorted by MAPK signature score (top), and expression of 10 signature genes (middle) for those cells. The 10 signature genes were selected as those that have the highest correlation with the average of all MAPK signature genes within each tumor. Shown are the five tumors with a significant correlation of MAPK signature genes: melanoma 88 (C), 81 (D), 80 (E), 78(F) and 71 (G).

FIG. 14A-14B depicts an analysis of TCGA bulk tumors and supports a connection between MAPK and MITF signaling in the context of NRAS mutant melanoma. MAPK signature genes were first restricted to those that were correlated in our single cell analysis; Applicants included only the genes that were among the top 10 correlated in at least two of the five tumors shown in FIG. 13. The average expression of those genes was defined as a MAPK signature score. Panel A: The distributions of MAPK signature score (shown by box-plots) are compared between tumors with wild-type (WT) and mutant (Mut) NRAS. This comparison was done separately among tumors with high expression of the MITF program genes (top third of tumors) and those with low expression of the MITF program genes (bottom third of tumors). Applicants found a significant increase in MAPK scores (P=4*10⁻⁶, t-test) only within MITF-high tumors. Panel B: Same as (A) for comparison of NRAS mutants to BRAF mutants. The same effect is observed, i.e. higher MAPK scores in NRAS mutants than in BRAF mutants, albeit with lower significance (P=0.02).

FIG. 15 shows AXL/MITF immunofluorescence staining of tissue slides of Mel80, Mel81 and Mel79 (40× magnification) revealed presence of AXL-expressing and MITF-expressing cells in each sample. Consistent with single-cell RNA-seq inferred frequencies of each population, Mel80 contained rare AXL-expressing cells (red, cell membrane staining) and mostly malignant MITF-positive cells (green, nuclear staining), while malignant cells of Mel81 almost exclusively consisted of AXL-expressing cells. Mel79 had a mixed population with rare cells positive for both markers, all in agreement with the inferred single-cell transcriptome data.

FIG. 16 depicts AXL upregulation in a second cohort of post-treatment melanoma samples and mutual exclusivity with MET upregulation. Each point reflects a comparison between a matched pair of pre-treatment and post-relapse samples from Hugo et al. (66), where the X-axis shows expression changes in MET, and the Y-axis shows expression changes in the AXL program minus those of the MITF program. Note that some patients are represented more than once based on multiple post-relapse samples. Fourteen out of 41 samples (34%) shown in red had significant upregulation of the AXL vs. MITF program, as determined by a modified t-test as described in Methods; these correspond to at least one sample from half (9/18) of the patients included in the analysis. Eleven out of 41 samples (27%) shown in blue had at least 3-fold upregulation of MET; these correspond to at least one sample from a third (6/18) of the patients included in the analysis. Notably, the AXL and MET upregulated samples are mutually exclusive, consistent with the possibility that these are alternative resistance mechanism.

FIG. 17A-17B depicts (A) Flow cytometry gating strategy for the exemplary cell lines WM88 (AXL-low) and IGR39 (AXL-high). Cells were treated with increasing doses of dabrafenib (D) and trametinib (T) at indicated doses, which resulted in an increase in the AXL-high cell fraction in WM88, and no changes in IGR39. (B) While cell lines with very low portion of AXL-positive cells demonstrate an increased frequency of AXL-high cells (FIGS. 3E and F) with combined BRAF/MEK-inhibition, AXL-high cell lines show minimal to no changes.

FIG. 18A-18C depicts a summary of multiplexed single-cell immunofluorescence in seven CCLE cell lines before and after treatment with BRAF/MEK-inhibition. (A) Relative fraction (compared to DMSO-treatment) of AXL-high cells (y-axis) treated for 5 or 10 days with increasing doses (as indicated on x-axis) of BRAF-inhibition alone (with vemurafenib) or in combination with a MEK-inhibitor (trametinib) with a 10:1 ratio (vemurafenib:trametinib). In all cell lines with a baseline low-fraction of AXL-expressing cells (WM88, MELHO, COLO679 and SKMEL28), there was a significant dose-dependent increase in the AXL-high cell fraction with BRAF-inhibition alone (black bars), and more pronounced with combined BRAF/MEK-inhibition (yellow bars). Cell lines with a baseline high AXL-expressing cell fraction (A2058, IGR39 and 294T) showed either minimal changes in the AXL-high cell fraction, however. A2058 demonstrated a significant decreased in the AXL-positive fraction. Although an outlier in this experiment, this indicates that alternative mechanisms of resistance with low AXL expression (Hugo et al.; FIG. S9). (B) The increase in AXL-high cell fractions in the sensitive cell lines was correlated with a significant decrease of p-ERK indicating strong MAP-kinase pathway inhibition, and (C) a decrease in cell viability. Overall, these results indicate, that the increase in the AXL-high cell fraction was at least in part due to a selection process. Both effects were more pronounced when cells were treated with combined BRAF/MEK-inhibition compared BRAF-inhibition alone.

FIG. 19A-19B depicts exemplary images of multiplexed single-cell immunofluorescence quantitative analysis for (A) an AXL-low (WM88) and (B) AXL-high cell line (A2058). Treatment with a combination of vemurafenib (V) and trametinib (T) at indicated doses on the left resulted in a dose-dependent change in the AXL-high population. In WM88, increasing drug concentrations led to killing of MITF-expressing, resulting in the emergence of a pre-existing AXL-high subpopulation. This indicates that the shift towards a higher AXL-expressing population (and possibly the AXL-high signature) is at least in part due to a selection process. While cell lines with a high baseline fraction of AXL-expressing cells showed modest to no changes in the AXL-fraction (FIG. 17B), A2058 was an exception. This cell lines has a major AXL-expressing population at baseline, which decreases with treatment, while the MITF-expressing population emerges. This indicates the presence of alternative mechanisms of resistance to RAF/MEK-inhibition, consistent with a recent report by Hugo et al. and our analysis shown in FIG. 16.

FIG. 20 depicts the identification of cell-type specific genes in melanoma tumors. Shown are the cell-type specific genes (rows) as chosen from single cell profiles (Methods), sorted by their associated cells cell type, and their expression levels (log 2(TPM/10+1)) across non-malignant and malignant tumor cells, also sorted by type (columns).

FIG. 21A-21B depicts the association of immune and stroma abundance in melanoma with progression-free survival.

FIG. 22A-22B shows the association between a malignant AXL program and CAFs. (A) Average expression (log 2(TPM+1)) of the AXL program (Y-axis) as defined here (bottom) and by Hoek et al. (top) in CAFs and melanoma cells from our tumors (this work, black bars) and in foreskin melanocytes and primary fibroblasts from the Roadmap Epigenome project (grey bars). Melanoma cells were partitioned to those from AXL-high and MITF-high tumors as marked in FIG. 3A. (B) CAF expression correlates with higher AXL program than MITF program expression in melanoma malignant cells. Scatter plot shows for each gene (dot) from the MITF (blue) or AXL (red) programs (as defined based on single-cell transcriptomes) the correlation of its expression with inferred CAF frequency across bulk tumors (Y-axis, from TCGA transcriptomes), and how specific its expression is to CAFs vs. melanoma malignant cells (X-axis, based on single-cell transcriptomes). Black dots indicate the expected correlations at each value of the horizontal axis as defined by a LOWESS regression over all genes. The average correlation values of MITF program genes are significantly lower than those of all genes and the correlation values of A×L program genes are significantly higher than those of all genes, even after restricting the analysis to melanoma-specific genes (X-axis <−2, P<0.01, t-test). A subset of AXL-program genes are specifically expressed in melanoma cells (but not CAFs) based on the single cell expression profiles, but associated with CAF abundance in bulk tumors (marked by red squares and gene names). MITF is negatively correlated with CAF abundance (R=−0.42) and is also indicated by gene name.

FIG. 23A-23B depicts immune modulators preferentially expressed by in-vivo CAFs. Panel A shows average expression levels of a set of immune modulators, including those shown in FIG. 4, in the five non-malignant cell types as defined by single cell analysis in melanoma tumors. Panel B shows a correlation of the set of immune modulators shown in (A) with inferred abundances of non-malignant cell type across TGA melanoma tumors.

FIG. 24A-24C depicts the identification of putative genes underlying cell-to-cell interactions from analysis of single cell profiles and TCGA samples. Applicants searched for genes that underlie potential cell-to-cell interactions, defined as those that are primarily expressed by cell type M (as defined by the single cell data) but correlate with the inferred relative frequency of cell type N (as defined from correlations across TCGA samples). For each pair of cell types (M and N), Applicants restricted the analysis to genes that are at least four-fold higher in cell type M than in cell type N and in any of the other four cell types. Applicants then calculated the Pearson correlation coefficient (R) between the expression of each of these genes in TCGA samples and the relative frequency of cell type N in those samples, and converted these into Z-scores. The set of genes with Z>3 and a correlation above 0.5 was defined as potential candidates that mediate an interaction between cell type M and cell type N. (A) Of all the pairwise comparisons Applicants identified interactions only between immune cells (B. T, macrophages) and non-immune cells (CAFs, endothelial cells, malignant melanoma) cells, such that the expression of genes from non-immune cells correlated with the relative frequency of immune cell types. Each plot shows a single pairwise comparison (M vs. N), including interactions of non-immune cell types (endothelial cells: left; CAFs: middle; malignant melanoma: right) with each of T-cells (A), B-cells (B) and macrophages (C). Each plot compares for each gene (dot) the relative expression of genes in the two cell types being compared (M-N) and the correlations of these genes' expression with the inferred frequency of cell type N across bulk TCGA tumors. Dashed lines denote the four-fold threshold. Genes that may underlie potential interactions, as defined above, are highlighted.

FIG. 25A-25C depicts immune modulators expressed by CAFs and macrophages. (A) Pearson correlation coefficient (color bar) across TCGA melanoma tumors between the expression level of each of the immune modulators shown in FIG. 4B and additional complement factors with significant expression levels. (B) Correlations across TCGA melanoma tumors between the expression level of the genes shown in (A) and the average expression levels of T cell marker genes. (C) Average expression level (log 2(TPM+1), color bar) of the genes shown in (A) in the single cell data, for cells classified into each of the major cell types Applicants identified. These results show that most complement factors are correlated with one another and with the abundance of T cells, even though some are primarily expressed by CAFs (including C3) and others by macrophages. In contrast, two complement factors (CFI, C5) and the complement regulatory genes (CD46 and CD55) show a different expression pattern.

FIG. 26A-26C depicts unique expression profiles of in vivo CAFs. (A-B) Distinct expression profiles in in vivo and in vitro CAFs. Shown are Pearson correlation coefficient between individual CAFs isolated in vivo from seven melanoma tumors, and CAFs cultured from one tumor (melanoma 80). Hierarchical clustering shows two clusters, one consisting of all in vivo CAFs, regardless of their tumor-of-origin (marked in (A)), and another of the in vitro CAFs. (C) Unique markers of in vivo CAFs include putative cell-cell interaction candidates. Left: Heatmap shows the expression level (log 2(TPM+1)) of CAF markers (bottom) and the top 14 genes with higher expression in in-vivo compared to in-vitro CAFs (t-test). Right: average (bulk) expression of the genes in the in-vivo CAFs, in-vitro CAFs, and primary foreskin fibroblasts from the Roadmap Epigenome project. Potential interacting genes from FIG. 4B are highlighted in bold red.

FIG. 27A-27F depicts TMA analysis of complement factor 3 association with CD8+ T-cell infiltration, and control staining. Two TMAs (CC38-01 and ME208, shown in A, C, E and B, D, F, respectively) were used to evaluate the association between complement factor 3 (C3) and CD8 across a large number of tissues obtained by core biopsies of normal skin, primary tumors, metastatic lesions and NATs (normal skin with adjacent tumor). In both TMAs with a total of 308 core biopsies, Applicants observed high correlation between C3 and CD8 (R >0.8, shown in FIG. 4C for one TMA). To verify that this correlation is not due to technical effects in which some tissues stain more than others irrespective of the stains examined (e.g., due to variability in cellularity or tissue quality), Applicants normalized the values (% area, Methods) for both C3 and CD8 by those of DAPI staining. Indeed, Applicants found a non-random yet non-linear association between DAPI stains and either C3 (A, B), or CD8 (C, D), which were removed by subtracting a LOWESS regression, shown as red curves in panels A-D. The normalized C3 and CD8 values were not correlated with DAPI levels, yet maintained a high correlation with one another (E, F). R=0.86 and 0.74 for primary and normal skin in panel E (TMA CC38-01), and R=0.78, 0.86, 0.63 and 0.31 for primary melanomas, metastasis, NATs and normal skin in panel F (TMA ME208), respectively.

FIG. 28A-28B depicts cytotoxic and naïve expression programs in T cells. (A) Cell scores from a combined PCA of all T cells. Cells are colored as CD8+(red), CD4+(green), T-regs (blue) and unresolved (black) based on expression of marker genes (FIG. 5A, Methods). (B) Gene scores for PC1 from a PCA of CD8+ cells (x-axis) and PC2 from a PCA of CD4+ cells (Y-axis). Selected marker genes are highlighted, including genes known to be associated with cytotoxic/active (red), naïve (blue) and exhausted (green) T cell states.

FIG. 29 depicts the frequency of cycling cells in different subsets of T-cells. Shown is the frequency of cycling T cells (as identified based on the expression of G1/S and G2/M gene-sets; Methods) for different subsets of T cells, including Tregs. CD4+ cells separated into five bins of increasing activation (arrow below green bars), CD8+ cells separated into five bins of increasing activation (arrow below red bars), and active/cytotoxic CD8+ further partitioned into those with relatively high or low exhaustion, as shown in FIG. 5D. Asterisks denote subsets with significant enrichment or depletion of cycling cells across all cells from the same subset of CD4+ or CD8+ cells as defined by P<0.05 in a hypergeometric test. Cell cycle frequency is associated with activation state of CD8+ T-cells, as the first bin is significantly depleted and the fifth bin is significantly enriched. A similar trend is observed in CD4+ T-cells (no cycling cells in the first bin and highest frequency in fifth bin), although none of the CD4 bins was significantly depleted or enriched. Exhaustion was not associated with significant differences in cell cycle frequency (P=0.34, Chi-square test).

FIG. 30A-30B identifies activation-independent exhaustion programs. Panel A shows a partial correlation between the expression of five co-inhibitory receptors which are used as markers for exhaustion, controlled for their common correlation with the cytotoxic expression program, among CD8+ T-cells from melanoma 58 (left), melanoma 74 (middle) and melanoma 79 (right). Panel B identifies subsets of cells with high expression (red) and low expression (green) of the five exhaustion markers genes, among cells with a limited range of expression of the cytotoxic expression program.

FIG. 31A-31B depicts the exhaustion program in Mel75. PCA of 314 CD8 T-cells from Mel75 identified an exhaustion program in which the top scoring genes for PC1 included the five co-inhibitory receptors shown in FIG. 5B as well as additional exhaustion-associated genes (e.g., BTLA, CBLB). Applicants defined PC1-associated genes based on a correlation p-value of 0.01 (with Bonferroni correction for multiple testing, see Table 13). Cells were then ranked by the residual between average expression of these PC1-associated genes (referred to as the exhaustion program) and average expression of the cytotoxic genes shown in FIG. 5B (referred to as the cytotoxic program) using a LOWESS regression, as shown in FIG. 5D. Finally, for each gene, Applicants ranked its expression levels across the CD8 T-cells from Mel75 and converted these to rank scores between 0 and 1 such that the i highest-expressing cell received a rank score of i/314, where 314 represents the number of CD8 T cells from Mel75. (A) Exhaustion and cytotoxic program scores for ranked Mel75 CD8 T-cells, after applying a moving average with windows of 31 genes. (B) The heatmap shows expression ranks of PC1-associated genes across the CD8 T-cells from Mel75 cells, ranked as described above.

FIG. 32A-32E depicts tumor-specific exhaustion programs. (A) Heatmap shows the significance (−log 10(P-value)) of tumor-specific variation in exhaustion gene scores (log-ratio in high vs. low exhaustion cells) comparing each tumor to all other tumors combined, for the same genes (and the same order) as shown in FIG. 5F. The sign of significance values reflects the direction of change (positive values shown in red reflect higher exhaustion values compared to other tumors while negative values shown in green reflect lower exhaustion values compared to other tumors). Three values are shown for each tumor, corresponding to exhaustion scores based on the exhaustion gene-sets derived from Mel75 analysis (FIG. 32)(3, 4), respectively. (B) Number of genes with significant tumor-specific up- or down-regulation (FDR <0.05 in each tumor, based on median of the three exhaustion scores), divided to three classes (bars) based on the differences in overall expression level across CD8 T-cells of the different tumors (green: genes lower in the respective tumor by at least two fold. Red: genes higher in the respective tumor by at least two fold. Black: genes with less than two-fold difference. This demonstrates that most changes in exhaustion co-expression are not identified in bulk level analysis of the CD8 T-cells. (C-D) Bar plots showing the significance of tumor-specific variation, as in (A), for CTLA4 (C) and NFATC1 (D). Dashed lines indicate significance thresholds that correspond to P<0.05. (E) Heatmap (as in subfigure A) for the target genes of NFATC1(5).

FIG. 33A-33B depicts the detection of Mel74 expanded T-cell clones by TCR sequence. (A) Clustering of Mel75 cells by their TCR segment usage. TCR Similarity was defined as zero for any pair with at least one inconsistent allele (i.e. resolved in both cells but distinct among the two cells), and as −log 10(P) for any pair without inconsistent alleles, where P reflects the estimated probability of randomly observing this or a higher degree of segment usage similarity. P is equal to the product of the probabilities for the four TCR segments. P(i,j)=Pβv(i,j)*Pβj(i,j)*Pαv(i,j)*Pα(i,j). For each segment, the probability equals one if segment usage is unresolved in at least one of the cells of the pair, and otherwise (i.e., if the two cells have the same allele) the probability is 1/N, where N is the number of distinct alleles that were identified for that segment. The TCR usage of one exemplary cluster is indicated. (B) Mel75 cells were ordered by the average relative expression of Exhaustion and Cytotoxic genes, as shown in FIG. 5B, and the percentage of clonally expanded cells (i.e., belonging to the clusters indicated in A) is shown with a moving average of 20 cells, demonstrating the depletion of expanded T cells among cells with high cytotoxic and low exhaustion expression. Dashed line indicates the overall frequency of clonally expanded cells. Note that the top and bottom panels are aligned but that due to the use of a 20-cell moving average, the top panel can only start at the 11th cell and end at the 11th cell from the end.

FIG. 34 depicts that the identification of distinct co-expression programs may require single cell analysis. Schematic depicting how single-cell RNA-seq can distinguish two scenarios that are indistinguishable by bulk profiling. Across individual tumor cells (top), genes A and B are either positively (left) or negatively (right) correlated. In bulk tumor (middle), the average expression of A,B cannot distinguish the two scenarios, whereas co-expression estimates from single cell RNA-seq (bottom) do so.

FIG. 35A-35F Single-cell RNA-seq of cancer and non-cancer cells in six oligodendroglioma tumors. (a) Experimental workflow. (b,c) Copy-number variations (CNVs) inferred from single cell RNA-Seq. Rows: cells; columns: chromosomal locations (100 gene windows). Red: inferred amplification; blue: inferred deletion; white: normal karyotype. (b) CNV profiles inferred from single cell RNA-seq for each of six tumors (top panel) and measured by DNA whole-exome sequencing (WES) of five tumors (bottom panel). Top cluster (in top panel): non-tumoral cells that lack CNVs, 3 bottom clusters: remaining cells from each of the six tumors, with deletions of chromosomes 1p and 19q, as well as tumor-specific CNVs. MGH36 and MGH97 cells are ordered by their pattern of CNVs, indicating variability in the copy numbers of chromosomes 4, 11 and 12, with a zoomed in view on a fraction of cells in (c). (d) PCA of malignant cells. Shown are PC1 (X-axis) vs. PC2+PC3 (Y-axis) scores of cells from three tumors based on a single combined PCA. (e) AC-like and OC-like signatures. Relative expression of the genes most correlated positively (bottom) or negatively (top) with PC1, in cancer cells from each of the three tumors (marked as in (d)), ranked by PC1 scores. Selected AC and OC marker genes are highlighted. (f) Relative expression of the mice orthologs of genes most correlated positively (bottom) or negatively (top) with PC1 (as shown in (e)) in mice OCs and ACs (97) (log₂-ratio of the respective cell type compared to the average of four measured cell types: OC, AC, OPC and neurons). Abbreviations: AC: astrocyte; OC: oligodendrocyte.

FIG. 36A-36G Stemness expression program and a developmental hierarchy of oligodendroglioma cells. (a) Stemness program. Average relative expression of the genes most highly correlated with PC2+PC3 (top), as well as the selected AC and OC marker genes shown in FIG. 35e (bottom), in four subpopulations defined by PC scores: stem-like cells (high PC2+PC3, intermediate PC1); undifferentiated cells (undiff.; low PC2+PC3, intermediate PC1); OC-like (high PC1); AC-like (low PC1). Genes were sorted by their relative expression in the stem-like cells. (b) Stemness program genes are also expressed in early human brain development. Relative expression of putative stemness genes correlated with PC2/3 (top) and OC/AC marker genes (bottom) across 524 human brain samples from the Human Developmental Transcriptome in the Allen Brain Atlas. Samples are ordered in columns by age, from early prenatal (left) to adults (right). (c) The stemness program is correlated to those of mouse activated NSC and human NPCs. Pearson correlation coefficients between the expression of PC2/3 genes (rows) and expression programs of mouse NSC (left) and human NPC (right) across single cells from the respective datasets, the NSC expression program reflects activation, and is quantified by “pseudotime” as defined previously (111); the NPC program reflects PC1 scores from a PCA analysis of 340 NPCs (FIG. 47). (d) Inferred developmental hierarchy in oligodendroglioma cells. Lineage scores (OC-like vs. AC-like expression program; X-axis, Methods) and sternness scores (stem-like vs. OC/AC-differentiation expression program; Y-axis, Methods) of malignant cells from the six tumors. Gray lines indicate the backbone (Methods) used to quantify density in FIG. 37B, 38A-B. (e) Density of cells (color bar) from each tumor across the backbone of the hierarchy in (a). For each position in the backbone, colors indicate the fraction of cells in each tumor that are within a Euclidean distance of 0.3. (f) Fraction of cancer cells in each of the compartment. Shown is the fraction of cells assigned to the different tumor compartments (Y axis, Methods) based on either single cell RNA-seq (blue) or RNA-ISH (orange), (example RNA-ISH shown in (g)). Circles: individual tumors; square and error bars: average and standard deviation across tumors, respectively, showing general agreement between scRNA-Seq and IHC estimates. (g) Tissue staining. Immunohistochemistry for Glial Fibrillary Acidic Protein (GFAP) and OLIG2 highlights astrocytic and oligodendroglial lineage differentiation, respectively, in subpopulations of cells in oligodendroglioma sample MGH54 (two top left panels). In situ RNA hybridization (ISH) for astrocytic markers APOE (apolipoprotein E, arrowhead) and oligodendrocytic marker OMG (oligodendrocyte myelin glycoprotein, arrow) confirms expression of these two lineage markers in distinct cells in oligodendroglioma. The stem/progenitor markers SOX4 (SRY (sex determining region Y)-box4) and CCND2 (cyclinD2), arrowheads, are co-expressed in the same cells and are mutually exclusive with the lineage marker ApoE (arrow).

FIG. 37A-37E. Cell cycle is enriched in the stem/progenitor cells in oligodendroglioma. (a) Cell cycle classification. Classification of cells to non-cycling (black) and three categories of cycling cells (color-coded by approximated phase as shown in inset) based on the relative expression of gene-sets associated with G1/S (X-axis) and G2/M (Y-axis) phases of the cell cycle. Thin light blue cells have intermediate scores and thus might reflect either early G1 phase, or possibly arrested or non-cycling cells. Blue, green and red cells have more significant expression of cell cycle genes and are thus more confidently defined as cycling cells. (b-d) Only stem/progenitor cells are cycling. (b) Hierarchy plot, as in FIG. 36d for MGH54 cells, with confidently-cycling cells color-coded as in (a). For Light blue (less confident) cells and the other tumors see FIG. 48. (c) Hierarchy plot for the six tumors, with each cell color-coded based on the fraction of neighboring cells, as defined with a Euclidean distance of 0.3, that are cycling (including light blue cells). (d) Left: ISH for Ki-67 (cell cycle marker) and SOX4 (stemness marker) showing co-expression in rare cells (arrows). A non-cycling Sox4+ cells is also highlighted (arrowhead). Right: Double immunohistochemistry for the differentiation marker GFAP (red) and the proliferation marker Ki-67 (brown), showing that proliferating cells (arrowheads) do not express differentiation markers (arrows). (e) Correlation between the average expression of cell cycle (Y-axis) and that of stemness genes (X-axis) across molecularly defined (IDH mutations, chromosome 1p and 19q co-deletion, and absence of P53 and ATRX mutations) oligodendrogliomas (circles) profiled by TCGA with bulk RNA-seq. Average expression was defined by centering the log 2-transformed RSEM gene quantifications. Also shown are the linear least-square regression and Pearson correlation coefficient.

FIG. 38A-38J. Intra-tumor genetic heterogeneity and association with expression states. Cells were classified to genetic subclones based on CNVs (a,b) or point-mutations (c-e), and examined for differences in gene expression states. (a,b) Both CNV clones in MGH36 and in MGH97 span all 3 tumor compartments. (a) Two clones (green and gray) in MGH36 and MGH97 based on CNV inference mapped to the cellular hierarchy defined by lineage (x-axis) and stemness (Y axis) scores. (b) Percentages of cycling cells (X axis) and of stem/progenitor cells (Y axis) in clone 1 (green) and clone 2 (gray) of MGH36 (square) and MGH97 (diamond). (c,d) Different clones defined by point mutations span all three tumor compartments. (c) Clones inferred by mutation analysis of single cell RNA-seq reads. Each panel shows lineage (X-axis) and stemness (Y-axis) scores for cells, colored by their mutation status (red: detected by single cell RNA-seq reads; black: not detected). Top left corner: mutation name, expected (E) fraction of mutant cells by ABSOLUTE (35), and fraction of single cells were the mutation was observed (O). (d) Clones determined by single cell mutation-specific qPCR. As in (c) but showing a wild-type CIC allele detected (green), a mutant CIC allele detected (orange) or neither one detected (black). (e) An expression signature for CIC-mutant cells. Shown is a heatmap of relative expression levels for CIC-dependent genes (rows) in CIC-mutant (right columns) and CIC-wild-type (left columns) cells. Key gene names are marked on left. Cells were classified to genetic subclones based on CNVs (f,g) or point-mutations (h-j), and examined for differences in gene expression states. (f,g) Both CNV clones in MGH36 span all 3 tumor compartments. (f) Two clones in MGH36 based on CNV inference mapped to the cellular hierarchy defined by lineage (x-axis) and stemness (Y axis) scores. (g) Density (color bar) of all cells (top) or only cycling cells (bottom) from the two clones of MGH36 across the backbone of the hierarchy as shown in FIG. 36d. Colors indicate the fraction of cells within a Euclidean distance of 0.3. (h,i) Different clones defined by point mutations span all 3 tumor compartments. (h) Clones inferred by mutation analysis of scRNA-Seq reads. Each panel shows lineage (X-axis) and stemness (Y-axis) scores for cells, colored by their mutation status based on scRNA-Seq reads (red: detected by scRNA-Seq; black: not detected). Top left corner: mutation name, expected (E) fraction of mutant cells by ABSOLUTE (35), and fraction of single cells were the mutation was observed (O). Top right corner: tumor ID. (i) Clones determined by single cell mutation-specific qPCR. As in (f) but showing a wild-type CIC allele detected (green), a mutant CIC allele detected (orange) or neither one detected (black). (j) An expression signature for CIC-mutant cells. Shown is a heatmap of relative expression levels for CIC-dependent genes (rows) in CIC-mutant (right columns) and CIC-wild-type (left columns) cells. Key gene names are marked on left.

FIG. 39. Molecular characterization of oligodendroglioma and validation of CNVs. Shown are IHC (top left) and FISH (all other panels) in a representative tumor (MGH36). All of the cases retain ATRX protein expression by immunohistochemistry (IHC) (top left) and show loss of chromosomes arms 1p (bottom left) and 19q (top right) by FISH. In addition, tumor specific CNVs identified by single-cell RNA-seq were confirmed by FISH (e.g., loss of chromosome 4 in MGH36, bottom right panel).

FIG. 40. Statistics of single cell RNA-seq experiments. Shown are the distributions of the total number of sequenced paired-end reads per cell (gray) and of paired-end reads that were mapped to the transcriptome and used to quantify gene expression (black).

FIG. 41A-41B. Two populations of non-cancer cells identified in oligodendroglioma. (A) Selected genes that are differentially expressed among the two populations of normal cells that lack CNVs (FIG. 35B, top), including markers of microglia (top) and oligodendrocytes (bottom). (B) Expression programs in microglia cells from the three tumors. The heatmap shows relative expression of genes (rows) across microglia cells (columns). Above the dashed line are microglia markers expressed in all microglia cells and below the line are the genes of a microglia activation program, which is variably expressed, and includes cytokines, chemokines, early response genes and other immune effectors. This latter gene set might reflect a microglia activation program that could either be a general microglia program or potentially specific to the context of oligodendroglioma. Microglia cells (columns) are rank ordered by their relative expression of the activation program. The tumor of origin of each cell is color-coded at the top panel.

FIG. 42A-42D. Principal component analysis. (A) PC2 and PC3 are associated with intermediate values of PC. PC1 scores are shown along with PC2 (top) and PC3 (bottom) scores for cells in each of the three tumors profiled at high depth. Red line indicates local weighted regression (LOWESS) with a span of 5%, which demonstrates that PC2 and PC3 values tend to be highest in intermediate values of PC1 and to decrease in either high PC1 (i.e. OC-like cells) or low PC1 (i.e. AC-like cells). (B) Consistency of PCA across tumors. Shown are the Pearson correlations in gene loadings (over all analyzed genes) between the top three PCs in PCA of the three tumors profiled at high depth (y axis, as shown in FIG. 1) and the top four PCs in alternative PCA of either all six tumors (left), as well as of PCA of each individual tumor (right). PC1-3 are highly consistent between the three-tumor and six-tumor PCAs (R>0.9); PC1 is highly consistent (R>0.8) between the three-tumor analysis and all other analysis. (C) PC1 (x axis) and PC2+PC3 (y axis) scores of malignant cells from each of the three tumors profiled at intermediate depth, showing consistent patterns with those shown in FIG. 1d. (D) Distribution of differences in PC1 loadings between the original PCA and the shuffled PCA (see description in the Methods section, Principal component analysis) for all genes (black), OC-like genes (blue) and AC-like genes (green). This analysis demonstrates that OC-like and AC-like gene-sets are highly skewed in the original PCA and their loadings are not recapitulated by shuffled data reflecting the effect of complexity.

FIG. 43A-43C. OC-like, AC-like and stem-like cell clusters by hierarchical clustering. (A) Cell-cell correlation matrix based on all analyzed genes across all malignant cells in MGH54. Cells are ordered by average linkage hierarchical clustering, and colored boxes indicate distinct clusters. Clusters are marked based on the identity of differentially expressed genes as OC-like (blue), AC-like (yellow), cycling (pink) stem-like (purple) and intermediate cells that do not score highly for any of those expression programs (orange). (B) Top differently expressed genes. Shown is the average expression in each of the OC-like, AC-like, stem-like and intermediate cell clusters (columns) of differentially expressed genes (rows) defined by comparing cells from each of the OC-like, AC-like and stem-like clusters to cells from the remaining clusters with a two-sample t-test. Similar genes are highlighted as in PCA (FIG. 35): (OC-like: OMG, OLIG1/2, SOX8; AC-like: ALDOC, APOE, SOX9; Stem-like: SOX4/11, CCND2, SOX2). Stem-like genes also include CTNNB1, USP22, and MSI1. (C) Cell-cell correlation matrices, as in (A) for cells of MGH36 and MGH53. Boxes indicate OC-like and AC-like clusters.

FIG. 44A-44C. The stemness program in oligodendroglioma overlaps with expression programs of glioblastoma (GBM) cancer stem cells and normal neural stem/progenitor cells. (A) Overlap with human GBM stemness program. Applicants have previously (Patel et al. 2014) identified a GBM stemness program and determined the association of each gene with that program by the correlation between the expression of that gene and the average expression of the stemness program's genes across individual cells (“CSC gradient”) in each of five GBM tumors. Shown is the average correlation (X axis) of each analyzed gene (green dots) across the five cases and the p-values of those correlations as determined with a t-test (Y axis). Genes also identified in the oligodendroglioma stemness program (this work) are marked in black. Applicants considered genes with p<0.05 (marked by dashed line) and an average correlation above 0.1 as significant in the GBM analysis. Eight genes in the oligodendroglioma stemness program overlapped with the significant GBM genes, representing a significant enrichment (1.5*10⁴, hypergeometric test). (B) Correlation with mouse activated NSC program. Shown is the distribution of correlation values (X axis) of either all genes (gray) or genes from the oligodendroglioma stemness program (black) with the expression program of mice NSC activation states, as previously quantified by “pseudotime”, across single mouse NSCs (Shin et al. 2015). The average correlation of the NSC activation program genes with oligodendroglioma stemness genes is significantly higher than with all other genes (P=3*10⁻⁶; t-test). (C) Correlation with human NPC program. Shown is the distribution of correlation values (X axis) of either all genes (gray) or genes from the oligodendroglioma stemness program (black) with an expression program of human NPCs identified by PCA (FIG. 43). Each gene's correlation to the average expression of the NPC program genes was calculated across single human NPCs. The average correlation with oligodendroglioma stemness genes is significantly higher than with all other genes (P=2*10⁻³⁵, t-test).

FIG. 45. In vitro sphere forming assay in serum-free conditions. Spherogenic oligodendroglioma line BT54 (Kelly et al. 2010) with 1p/19q co-deletion and IDH1 mutation, was sorted for CD24 by flow cytometry and 20,000 cells were plated in serum-free medium supplemented with EGF and FGF, in duplicate (Methods). 14 days after sorting overall sphere formation was evaluated. Similar results were obtained in duplicate experiment. Representative example depicted.

FIG. 46. Preferential expression of the oligodendroglioma stemness program in neurons but not in OPCs. Genes expressed in the oligodendroglioma single cells were divided into six bins (bars) based on their relative expression (log₂-ratio) in stem-like cells with high PC2/3 and intermediate PC1 scores compared to all other cells. Bins were defined by expression intervals, (X-axis labels). Each panel shows for each bin the average relative expression in each of three normal brain cell types (Y axis) based on data from the Barres lab RNA-seq database (Zhang et al. 2014, Zhang et al. 2016): mice oligodendrocyte progenitor cells (mOPC, top), mouse neurons (mNeurons, middle), and human neurons (hNeurons, bottom). Relative expression of each gene in each CNS cell type was defined as the log₂-ratio between the respective cell type divided by the average over AC, OC and neurons. Error bars: standard error as defined by bootstrapping. Asterisks: bins with significantly different relative expression (in the respective normal cell type) compared to all genes expressed in oligodendroglioma, based on P<0.001 (by t-test) and average expression change of at least 30%.

FIG. 47A-47F. Analysis of human NPCs. (A-D) Differentiation potential of Human SVZ NPCs. Human SVZ NPCs isolated from 19 weeks old fetus form neurospheres in culture (A), and can be differentiated to neuronal (Neurofilament. B), oligodendrocytic (OLIG2, C), or astrocytic (GFAP, D) lineages in vitro. Scale bars: 25 um (A), 10 um (B-D). Applicants note that although OLIG2 can represent different cell types it is very lowly expressed in the fetal NPCs before differentiation (an average log 2(TPM+1) of 0.82, compared to a threshold of 4 that Applicants use to define expressed genes in our analysis, and zero cells with expression above this threshold). Thus, the undifferentiated NPCs do not express OLIG2 and Applicants interpret the expression of OLIG2 as a sign of oligodendroglial lineage differentiation. (E, F) Single cell RNA-Seq analysis of NPCs. (E) NPCs have an expression program similar to that of the oligodendroglioma stemness program; Heatmap shows the expression of genes (rows) most positively (top) or negatively (bottom) correlated with PC1 of a PCA of RNA-seq profiles for 431 single NPCs, across NPC cells (columns) rank ordered by their PC1 scores. Selected genes are indicated, and a full list of correlated genes for PC1 and PC2 is given in Table 19. (F) NPC cell scores for PC1 (Y-axis) and PC2 (X-axis). PC2 correlated genes (Table 19) are associated with the cell cycle. Cells with the highest PC1 scores tend to be non-cycling (low PC2 score), indicating that while the sternness program is coupled to the cell cycle in oligodendroglioma, it is decoupled from the cell cycle in NPCs.

FIG. 48A-48B. Sternness and lineage score for individual tumors. (A) Shown are plots as in FIG. 37b for each of the six tumors. Cycling cells are colored as in FIG. 37, with G1/S cells in blue, S/G2 cells in green, G2/M cells in red, and potential early G1 cells in light blue. (B) Lineage and sternness scores for the three tumors with high-depth profiling, colored based on sequencing batches, demonstrating the lack of considerable batch effects.

FIG. 49A-49G. Single cell RNA-seq of MGH60 reveals similar hierarchy to that of MGH36, 53 and 54. A fourth oligodendroglioma tumor (MGH60) was profiled by two protocols for single cell RNA-seq: the full-length SMART-Seq2 protocol (a,b) used to generate all single cell RNA-seq of MGH36, 53 and 54; and an alternative protocol (c,d) where only the 5′-ends of transcripts are analyzed while incorporating random molecular tags (RMTs, also known us unique molecular identifiers, or UMIs) that decrease the biases of PCR amplification. The same tumor was also analyzed by whole exome sequencing (e). (a,c) In data from both protocols. PC1 reflects an AC-like and OC-like distinction. Shown are heatmaps of the AC-like and OC-like specific genes (rows, as defined in Table 18 and restricted to genes with average expression log 2(TPM+1)>4 in each dataset) with cells ordered by their PC score. (b,d,e,f) In data from both protocols, Applicants observe a developmental hierarchy. Shown are the cells analyzed by each protocol by their lineage (X axis) and stemness (Y axis) scores (defined as in FIG. 36E). Cycling cells were found only in the cells analyzed by SMART-seq2, due to the limited number of sequenced cells with the 5′-end protocol, and are shown to be specific to stem/progenitor-like cells, as observed for the other three tumors (FIG. 37). (g) Copy number profiles of MGH60 cells as inferred from single cell RNA-seq (top panel), and as measured by WES (bottom panel), demonstrating the consistency between these approaches.

FIG. 50A-50B. Characterization of tumor subpopulations by histopathology and tissue staining. (A) Two predominant lineages of AC-like and OC-like cells. Shown is MGH53 with hematoxylin and Eosin (H&E, top left), immunohistochemistry for OLIG2 (oligodendrocytic lineage marker, top right) and GFAP (astrocytic marker, bottom left), as well as in situ RNA hybridization for astrocytic markers ApoE (apolipoprotein E, bottom right), with patterns similar to GFAP immunohistochemistry. (B) Cycling cells are enriched among stem-like cells. In situ RNA hybridization for the stem/progenitor markers SOX4 (left panel) and the proliferation marker Ki-67 (right panel) in MGH36 identifies cells positive for both markers (arrows). Immunohistochemistry for GFAP (arrowhead, right panel) and Ki-67 (arrow, right panel) in MGH36 shows mutually exclusive expression patterns.

FIG. 51A-51E. Cycling cancer cells identified by scoring G1/S and G2/M associated gene-sets. (A) A cell cycle trajectory. Shown are cells (dots) scored by the average levels of gene expression of genes-sets associated with G1/S (X axis) and G2/M (Y axis) (Methods). Cells were then rank ordered by identifying all putative cycling cells with at least a 2-fold upregulation and a 1-test P-value <0.01 for either the G1/S or the G2/M gene-set, then manually partitioning those cells to distinct regions (color code), and finally estimating the direction of cell cycle progression in each region and ordering the cells in that region accordingly (edges; Methods). (B-E) High expression of GUS and G2/M gene sets in distinct cycling cells. Shown is the average expression of GU/S (blue curve in B, D; top genes in C, E) and G2/M (green curve in B. D; bottom genes in C. E) genes in all cells (B,C) or only the putative cycling cells (D, E). Cells are rank ordered as in (A). Dashed lines in (D) separate the four subsets of cycling cells, corresponding to light blue, blue, green and red in (A).

FIG. 52A-52C. Agreement in proportion of cycling cells estimated from single-cell RNA-seq and Ki-67 staining. (A, B) Estimated proportion of cycling cells agrees between single cell RNA-Seq and Ki-76 immunohistochemistry. Shown are the estimates of proportion of cycling cells (Y axis) in each of 3 tumors (X axis) based on single cell RNA-Seq (A; different phases assessed by color code as in FIG. 51a) or Ki-67 immunohistochemistry (B). (C) Variation in cycling cells between regions of the same tumor. Shown is Ki-67 immunohistochemistry in two regions in MGH36. Such regional variability in proliferation complicates direct comparisons.

FIG. 53A-53C. Enrichment of cycling cells among stem-like and undifferentiated oligodendroglioma cells. (A,B) Cycling cells are enriched in stem-like and undifferentiated cells compared to differentiated cells. Shown is the percentage of cycling cells (Y axis) in oligodendroglioma cells divided into four bins based on stemness scores (A, Methods) or based on lineage scores (B, Methods). Black squares and error-bars correspond to the mean and standard deviation of the percentages in the three tumors profiled at high depth (MGH36, MGH53, MGH54), and red circles denote the percentages in individual tumors. The four bins in (A) correspond to stemness scores below −1.5 (n=711), between −1.5 and 0.5 (n=1,100), between −0.5 and 0.5 (n=939), and above 0.5 (n=274), respectively. The first two bins are significantly depleted with cycling cells, while the last two bins are significantly enriched (P<0.05, hypergeometric test). The five bins in (B) correspond to AC score above 1 (n=503), AC score between 0.5 and 1 (n=1013), AC and OC scores below 0.5 (n=1130), OC score between 0.5 and 1 (n=855), and OC score above 1 (n=597), respectively. The third bin is significantly enriched with cycling cells, while the four other bins are significantly depleted (P<0.05, hypergeometric test). (C) Specific enrichment of S/G2/M cells compared to G1 cells among stem-like or undifferentiated cells. Shown is the proportion (Y axis) of each marked category of cells among the stem-like or undifferentiated subpopulations. Significant enrichments are marked (P<0.01, hypergeometric test).

FIG. 54A-54D. CCND2 is associated with both cycling and non-cycling stem/progenitor cells. (A) CCND2, but not CCND1/3, is upregulated in non-cycling stem-like oligodendroglioma cells. Shown are the average expression levels (Y axis, log-scale) of three cyclin-D genes (X axis) in non-cycling cells classified as OC-like cells (light blue), undifferentiated cells (gray) and stem-like cells (purple). CCND2 is ˜4-fold higher in stem-like non-cycling cells than in OC-like and undifferentiated cells (P<0.001 by permutation test). Conversely, CCND1 and CCND3 are expressed at comparable levels in stem-like and OC-like cells. (B) Up-regulation of cyclin-D genes in cycling cells compared to non-cycling cells. As in (A) but for up regulation (log₂-ratio) in cycling cells vs. non-cycling cells. CCND2 levels further increase in cycling undifferentiated and stem-like cells but not in OC-like cells, while CCND1 and CCND3 levels increase in OC-like cycling cells more than in undifferentiated and stem-like cycling cells. (C) Distinct expression pattern of cyclin D genes in human brain development. Shown are the expression pattern of three cyclin-D genes (rows) in human brain samples at different points in pre- and post-natal development, sorted by age (columns; pre/post to left/right of dashed vertical line) from the Allen Brain Atlas (Miller et al.). CCND2 is associated with prenatal samples, whereas CCND1 and CCND3 are expressed mostly in childhood and adult samples. (D) CCND2 is upregulated in activated vs. quiescent NSCs (Shin et al. 2015) both among cycling and non-cycling cells. Activated NSCs were partitioned into non-cycling cells (black) and cycling cells in the G1/S (green) or G2/M (red) phases (Methods). Expression difference (Y axis) for each of three genes (X axis) was quantified for each of these subsets as the log₂-ratio of the average expression in the respective subset vs. the quiescent NSCs, and was significant for each of the three subsets (P<0.05 by permutation test). While CCND2 (left) is induced in both cycling and non-cycling activated NSCs, two canonical cell cycle genes (PCNA; middle, and AURKB, right) are not induced in non-cycling genes but were induced preferentially in G1/S and G2/M cells, respectively.

FIG. 55. Distribution of cellular states in distinct genetic clones of MGH36 and MGH97. (A) Shown are sternness (Y axis) and lineage (X axis) score plots for MGH36 (top) and MGH97 (bottom), each separated into clone 1 (left) and clone 2 (right) as determined by CNV analysis (FIG. 35b,c). Cycling cells are colored as in FIG. 37, with G1/S cells in blue. S/G2 cells in green, and G2/M cells in red. (B) Color-coded density of cells across the cellular hierarchy as shown in FIG. 36e, for the two clones (left: clone 1, right: clone 2) in each of the two tumors (top: MGH36, bottom: MGH97).

FIG. 56. Multiple subclonal mutations each span the cellular hierarchy. Each panel shows lineage (X axis) and stemness (Y axis) scores of cells in which Applicants ascertained by single cell RNA-seq a mutant (red), a wild-type (blue) or none (black) of the alleles. Included are mutations for which at least three cells were identified as mutants and that were identified by WES as subclonal (fraction <60%). The gene names, tumor name, ABSOLUTE-derived fraction of mutant cells (E, for Expected fraction) and the fraction of cells detected as mutant by RNA-seq (0, for Observed) are also indicated within each panel. Applicants note that identification of a wild-type allele (blue) does not imply a wild-type cell because mutations may be heterozygous and thus cells could contain both alleles while only one may be detected by single cell RNA-seq. The observed fraction of mutations (0) is much lower than expected (E) due to limited coverage of the single cell RNA-seq data as well as due to heterozygosity. The vast majority of mutations (20 of 22) are distributed across the hierarchy and span multiple compartments. Two remaining mutations (H2AFV and EIF2AK2) appear more restricted to the “undifferentiated” region (intermediate lineage and stemness scores), which could reflect our limited detection rate of mutant cells and/or a bias of the mutation to a particular region. To test the significance of potential biases in the distribution of mutations Applicants calculated, for each mutation, a Euclidean distance among all pairs of mutant cells (based on their lineage and stemness scores), and compared the average pairwise distances among mutant cells to that among randomly selected subsets of the same number of cells. None of the mutations were significant with a false discovery rate (FDR) of 0.1, although this could reflect our limited statistical power and Applicants cannot exclude a potential bias. Applicants note, however, that even if a subset of mutations are biased in their distribution (as Applicants show for clone 1 in MGH36, FIG. 38a,b), the wide distribution of expression states for most mutations, as well as for the CNV clones (FIG. 38a,b) and for the LOH-clones (FIG. 57), is highly inconsistent with a model in which the hierarchy is driven by genetics, which would predict that all low-frequency subclones would be restricted to regions of the hierarchy, as Applicants discuss in FIG. 58. The apparent bias of mutant cells to the OC lineage over the AC lineage (i.e. positive vs. negative lineage scores) reflects the lower frequencies of AC-like cells compared to OC-like cells in MGH53 and MGH54 (MGH53: 17% AC vs. 39% OC; MGH54: 23% AC vs. 45% OC); this bias is also observed for the detection of wild-type alleles (blue) further demonstrating that there is no bias against mutation detection in the AC lineage.

FIG. 57A-57B. Loss-of-heterozygosity (LOH) event in MGH54 reveals two clones that span the cellular hierarchy. (A) Chromosome 18 LOH in MGH54. Allelic fraction analysis of MGH54 SNPs from WES shows an imbalance (red and blue dots) in the frequency of alternative alleles in chromosome 1p, 19q, as well as chromosome 18, despite the normal copy number at this chromosome (FIG. 35B). This is consistent with an LOH event in which presumably one copy of chromosome 18 was deleted, and the other copy amplified. The weaker imbalance compared to chromosomes 1p and 19q further indicates that this is a subclonal event. (B) Each of two clones defined by Chr. 18 LOH status spans the full hierarchy. Shown are the lineage (X axis) and stemness (Y axis) scores for each cell from MGH54 classified as pre-LOH (red), post-LOH (blue) and unresolved (black) based on RNA-seq reads that map to SNPs in the minor (i.e. deleted) chromosome. Both the pre- and post-LOH clones span the different tumor subpopulations. Pre-LOH cells were defined as all cells with reads that map to minor alleles in chromosome 18; post-LOH cells were defined as all cells with reads that map to at least five different major alleles, but no reads that map to minor alleles in chromosome 18; all other cells were defined as unresolved.

FIG. 58A-58E. The observed distribution of mutations is highly inconsistent with a model of genetically-driven hierarchy. (A) Phylogenetic tree for a hypothetical tumor, where each circle correspond to a cell. Six subclonal mutations are shown (black arrows), each defining a genetic subclone. (B) Under a genetically-driven hierarchy, specific subclones would correspond to subpopulations with distinct expression states, such that all cells in those subclones map into a specific expression state. Shown are schemes of the cellular hierarchy in oligondroglioma (i.e. the two lower branches reflect the AC-like and OC-like lineages and the top part reflect stem-like cells), with cells from a given subclone marked in red and confined to specific transcriptional states. Importantly, the restriction of a subclone to a specific expression state holds true not only for the subclones which are defined by the mutation that is causal for an expression state but also for any other subclone that is contained within it. For example, assuming that subclones 1 and 4 reflect the mutations that are causal for the OC-like and AC-like expression states, subclones 2 and 5 would also be confined to either the OC-like or the AC-like states. This is especially true for small subclones (i.e., mutations with a low clonal fraction), as these should be confined to a small branch in the phylogenetic tree that is unlikely to cover multiple subpopulations. Small subclones that nevertheless cover all three subpopulations are especially unlikely by this model, although these are observed in the data (e.g. ZEB2, FRG1, FTH1 and EEF1B2 in FIG. 38c all have a clonal fraction of 11% or less but span the three compartments of the hierarchy). Such cases could theoretically be explained by an identical mutation that occurs independently in multiple branches and thereby covers small subsets of cells from multiple branches. However, this is highly unlikely to account for the mutations that Applicants observe, as none of these mutations with the potential exception of the CIC mutation is a known “hot-spot” mutation that is expected to recur (and even the specific CIC mutation Applicants find is one of many mutations for this gene, and reported for 4 of 66 CIC-mutated TCGA patient samples). Thus, even convergent evolution is unlikely to result in these mutations occurring independently in different branches of the phylogenetic tree. Furthermore, Applicants identified three cases of compound chromosomal aberrations (two concurrent chromosomal deletions in MGH36, a chromosomal deletion and gain in MGH97, and a chromosome-wide LOH in MGH54 that requires two distinct genetic events) that in each case define two distinct clones, each of which spanning the different expression-based subpopulations; these events are highly unlikely to occur independently in different branches. (C) Under a non-genetic driven hierarchy, individual subclones tend to span the different expression states represented by the cellular hierarchy, consistent with the data herein. Applicants note that this model does not exclude the possibility that subclones would be biased towards (or against) a certain cellular state, as genetic evolution could interact with non-genetic states and influence their prevalence. (D) Phylogenetic tree for a hypothetical tumor, where each circle correspond to a cell. According to the model of genetically-driven hierarchy, specific regions in the tree would correspond to subpopulations with distinct expression states. Shown are examples of three such potential subpopulations. (E) Mutations acquired during tumor evolution (numbered arrows) generate tumor subclones that harbor these mutations (indicated as numbered circles) and are confined to specific branches of the tree. Therefore, according to the model of genetically-driven hierarchy, subclonal mutations are expected to be present only in cells from a specific subpopulation, as defined by expression states. This is especially true for small subclones (i.e. mutations with a low clonal fraction), as these should be confined to a small branch that is unlikely to cover multiple subpopulations. Small subclones that nevertheless cover all three subpopulations are especially unlikely by this model (such as ZEB2, FRG1 and EEF1B2 shown in FIG. 38; all with clonal fraction of 11% or less but span the three compartments of the hierarchy). Such cases could theoretically be explained by an identical mutation that occurs independently in multiple branches and thereby covers small subsets of cells from multiple branches. However, this is highly unlikely to account for the mutations that Applicants observe, as none of these mutations, except for CIC, is a known “hot-spot” mutation that is expected to recur. Thus, even convergent evolution is unlikely to result in these mutations occurring independently in different branches of the phylogenetic tree. Furthermore, Applicants identified two cases of large chromosomal aberrations (two concurrent chromosomal deletions in MGH36, and a chromosome-wide LOH in MGH54) that in each case define two distinct clones, and each of which spans the different expression-based subpopulations; these events are highly unlikely to occur independently in different branches.

FIG. 59. Model for oligodendroglioma architecture and clonal evolution. Early in their pathogenesis (left), tumors are composed of a single genetic clone and hierarchically organized, such that a subpopulation of cycling stem/progenitor cells gives rise to differentiated progeny in two glial lineages. As the tumor evolves (right), multiple genetic clones are generated and co-exist, with each genetic clone maintaining a hierarchical organization where the relative distribution of the different compartment may vary due to genetic effects but is overall similar.

FIG. 60 depicts expression of complement genes in microglia cells in breast metastases in the brain. Heatmap shows the expression level of indicated genes (x-axis) in single microglia cells (y-axis).

FIG. 61 depicts expression of complement genes in T cells in breast metastases in the brain. Heatmap shows the expression level of indicated genes (x-axis) in single T cells (y-axis).

FIG. 62 depicts expression of immune regulatory genes in T cells in breast metastases in the brain. Heatmap shows the expression level of indicated genes (x-axis) in single T cells (y-axis).

FIG. 63 depicts expression of complement genes in tumor cells in breast metastases in the brain. Heatmap shows the expression level of indicated genes (x-axis) in single tumor cells (y-axis).

FIG. 64 depicts the expression of complement genes by CAFs and macrophages in head and neck squamous cell carcinoma (HNSCC). 2150 single cells from 10 HNSCC tumors were profiled by single cell RNA-seq and were classified into 8 cells types based on tSNE analysis, as described herein for melanoma tumors. Shown are the average expression levels (log 2(TPM+1), color coded) of complement genes (Y-axis) in cells from each of the 8 cell types, demonstrating high expression of most complement genes by fibroblasts or macrophages, consistent with the patterns found in melanoma analysis. The predicted cell types (X-axis) are T-cells, B-cells, macrophages, mast cells, endothelial cells, myofibroblasts, CAFs, and malignant HNSCC cells; the number of cells classified to each cell type is indicated in parenthesis (X-axis).

FIG. 65. For each of the three tumors profiled at high depth (horizontal panels) and for the two lineages (vertical panels) Applicants calculated the significance of co-expression among sets of AC-related and OC-related genes within limited ranges of lineage scores (between the value of the X axis and that of the Y axis). Significance was calculated by comparison to 100,000 control gene-sets with similar number of genes and distribution of average expression levels, and is indicated by color. The significant co-expression patterns within limited ranges of lineage scores suggest that variability of lineage scores in these ranges cannot be driven by noise alone, and implies the existence of multiple states within each lineage, presumably reflecting intermediate differentiation states (see Note 2).

### DETAILED DESCRIPTION

The invention relates to gene expression signatures and networks of tumors and tissues, as well as multicellular ecosystems of tumors and tissues and the cells and cell type which they comprise. The invention provides methods of characterizing components, functions and interactions of tumors and tissues and the cells which they comprise.

The invention further relates to controlling an immune response by modulating the activity of a component of the complement system. Cancer is but a single exemplary condition that can be controlled by an immune reaction. The present invention describes for the first time how complement expression in the microenvironment can control the abundance of immune cells at a site of disease or condition requiring a shift in balance of an immune response.

The invention provides signature genes, gene products, and expression profiles of signature genes, gene networks, and gene products of tumors and component cells, and including especially melanoma tumors, gliomas, head and neck cancer, brain metastases of breast cancer, and tumors in The Cancer Genome Atlas (TCGA) and tissues. This invention further relates generally to compositions and methods for identifying genes and gene networks that respond to, modulate, control or otherwise influence tumors and tissues, including cells and cell types of the tumors and tissues, and malignant, microenvironmental, or immunologic states of the tumor cells and tissues. The invention also relates to methods of diagnosing, prognosing and/or staging of tumors, tissues and cells, and provides compositions and methods of modulating expression of genes and gene networks of tumors, tissues and cells, as well as methods of identifying, designing and selecting appropriate treatment regimens.

### Use of Signature Genes

As used herein a signature may encompass any gene or genes, protein or proteins, or epigenetic element(s) whose expression profile or whose occurrence is associated with a specific cell type, subtype, or cell state of a specific cell type or subtype within a population of cells. Increased or decreased expression or activity or prevalence may be compared between different cells in order to characterize or identify for instance specific cell (sub)populations. A gene signature as used herein, may thus refer to any set of up- and down-regulated genes between different cells or cell (sub)populations derived from a gene-expression profile. For example, a gene signature may comprise a list of genes differentially expressed in a distinction of interest. It is to be understood that also when referring to proteins (e.g. differentially expressed proteins), such may fall within the definition of “gene” signature.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. blood samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cancer cells that are linked to particular pathological condition (e.g. cancer grade), or linked to a particular outcome or progression of the disease, or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular tumor cell or tumor cell (sub)population if it is upregulated or only present, detected or detectable in that particular particular tumor cell or tumor cell (sub)population, or alternatively is downregulated or only absent, or undetectable in that particular particular tumor cell or tumor cell (sub)population. In this context, a signature consists of one or more differentially expressed genes/proteins or differential epigenetic elements when comparing different cells or cell (sub)populations, including comparing different tumor cells or tumor cell (sub)populations, as well as comparing tumor cells or tumor cell (sub)populations with non-tumor cells or non-tumor cell (sub)populations. It is to be understood that “differentially expressed” genes/proteins include genes/proteins which are up- or down-regulated as well as genes/proteins which are turned on or off. When referring to up-or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of tumor cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

Signatures may be functionally validated as being uniquely associated with a particular immune responder phenotype. Induction or suppression of a particular signature may consequentially associated with or causally drive a particular immune responder phenotype.

Various aspects and embodiments of the invention may involve analyzing gene signatures, protein signature, and/or other genetic or epigenetic signature based on single cell analyses (e.g. single cell RNA sequencing) or alternatively based on cell population analyses, as is defined herein elsewhere.

In further aspects, the invention relates to gene signatures, protein signature, and/or other genetic or epigenetic signature of particular tumor cell subpopulations, as defined herein elsewhere. The invention hereto also further relates to particular tumor cell subpopulations, which may be identified based on the methods according to the invention as discussed herein; as well as methods to obtain such cell (sub)populations and screening methods to identify agents capable of inducing or suppressing particular tumor cell (sub)populations.

The invention further relates to various uses of the gene signatures, protein signature, and/or other genetic or epigenetic signature as defined herein, as well as various uses of the tumor cells or tumor cell (sub)populations as defined herein. Particular advantageous uses include methods for identifying agents capable of inducing or suppressing particular tumor cell (sub)populations based on the gene signatures, protein signature, and/or other genetic or epigenetic signature as defined herein. The invention further relates to agents capable of inducing or suppressing particular tumor cell (sub)populations based on the gene signatures, protein signature, and/or other genetic or epigenetic signature as defined herein, as well as their use for modulating, such as inducing or repressing, a particular a particular gene signature, protein signature, and/or other genetic or epigenetic signature. In one embodiment, genes in one population of cells may be activated or suppressed in order to affect the cells of another population. In related aspects, modulating, such as inducing or repressing, a particular a particular gene signature, protein signature, and/or other genetic or epigenetic signature may modify overall tumor composition, such as tumor cell composition, such as tumor cell subpopulation composition or distribution, or functionality.

As used herein the term “signature gene” means any gene or genes whose expression profile is associated with a specific cell type, subtype, or cell state of a specific cell type or subtype within a population of cells. The signature gene can be used to indicate the presence of a cell type, a subtype of the cell type, the state of the microenvironment of a population of cells, and/or the overall status of the entire cell population. Furthermore, the signature genes may be indicative of cells within a population of cells in vivo. The signature genes of the present invention were discovered by analysis of expression profiles of single-cells within a population of cells from freshly isolated tumors, thus allowing the discovery of novel cell subtypes that were previously invisible in a population of cells within a tumor. The presence of subtypes may be determined by subtype specific signature genes. The presence of these specific cell types may be determined by applying the signature genes to bulk sequencing data in a patient tumor. Not being bound by a theory, a tumor is a conglomeration of many cells that make up a tumor microenvironment, whereby the cells communicate and affect each other in specific ways. As such, specific cell types within this microenvironment may express signature genes specific for this microenvironment. Not being bound by a theory the signature genes of the present invention may be microenvironment specific, such as their expression in a tumor. Not being bound by a theory, signature genes determined in single cells that originated in a tumor are specific to other tumors. Not being bound by a theory, a combination of cell subtypes in a tumor may indicate an outcome. Not being bound by a theory, the signature genes can be used to deconvolute the network of cells present in a tumor based on comparing them to data from bulk analysis of a tumor sample. Not being bound by a theory the presence of specific cells and cell subtypes are indicative of tumor growth and resistance to treatment. The signature gene may indicate the presence of one particular cell type. In one embodiment, the signature genes may indicate that tumor infiltrating T-cells are present. The presence of cell types within a tumor may indicate that the tumor will be resistant to a treatment. In one embodiment the signature genes of the present invention are applied to bulk sequencing data from a tumor sample to transform the data into information relating to disease outcome and personalized treatments. In one embodiment, the novel signature genes are used to detect multiple cell states that occur in a subpopulation of tumor cells that are linked to resistance to targeted therapies and progressive tumor growth.

In one embodiment, the signature genes are detected by immunofluorescence, by mass cytometry (CyTOF), drop-seq, single cell qPCR, MERFISH (multiplex (in situ) RNA FISH) and/or by in situ hybridization. Other methods including absorbance assays and colorimetric assays are known in the art and may be used herein.

In one embodiment, tumor cells are stained for cell subtype specific signature genes. In one embodiment the cells are fixed. In another embodiment, the cells are formalin fixed and paraffin embedded. Not being bound by a theory, the presence of the cell subtypes in a tumor indicate outcome and personalized treatments. Not being bound by a theory, the cell subtypes may be quantitated in a section of a tumor and the number of cells indicates an outcome and personalized treatment.

It will be understood by the skilled person that treating as referred to herein encompasses enhancing treatment, or improving treatment efficacy. Treatment may include tumor regression as well as inhibition of tumor growth or tumor cell proliferation, or inhibition or reduction of otherwise deleterious effects associated with the tumor.

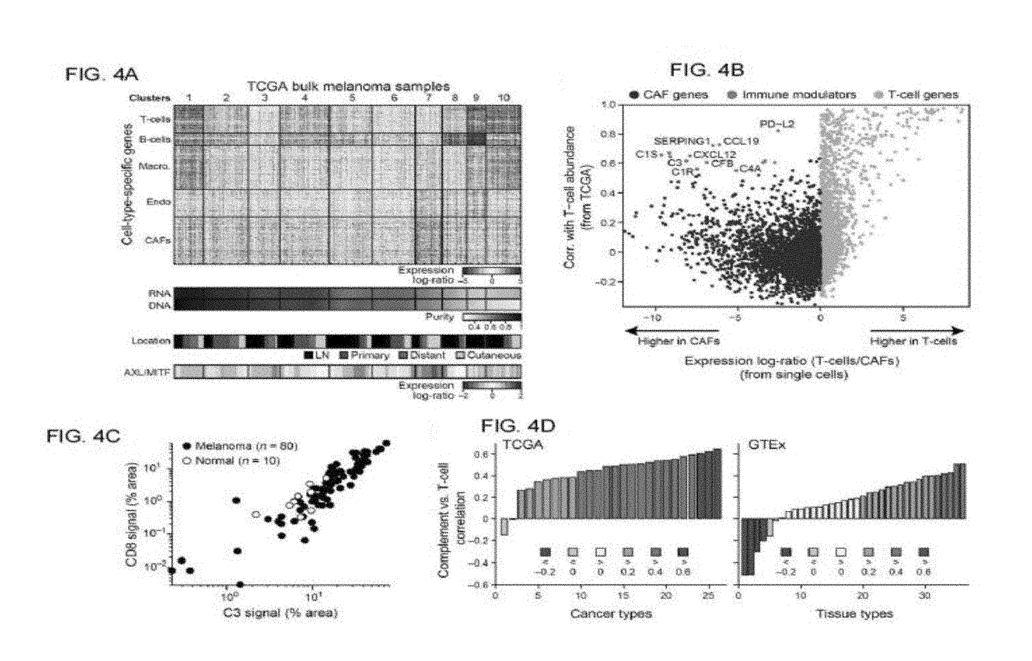

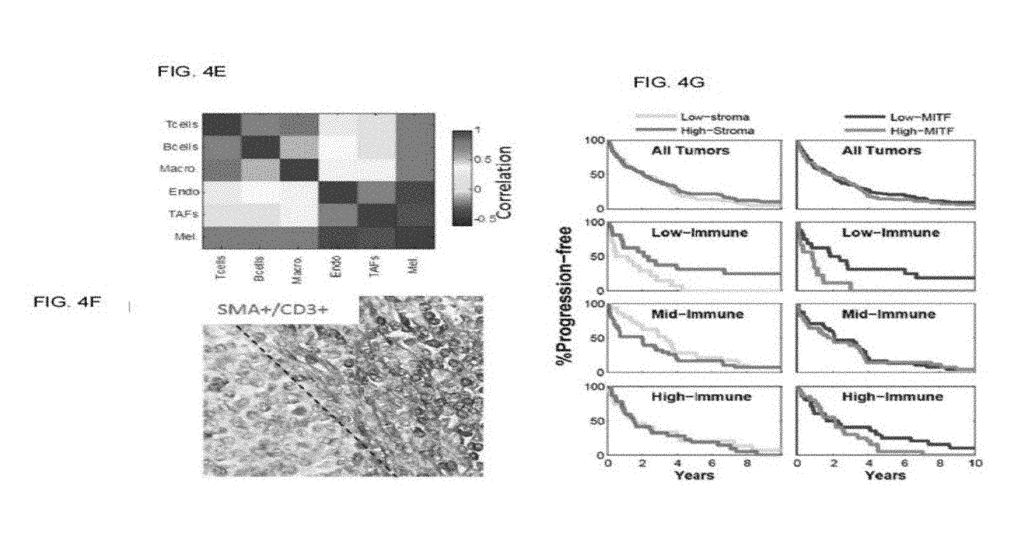

Immune checkpoints are inhibitory pathways that slow down or stop immune reactions and prevent excessive tissue damage from uncontrolled activity of immune cells. By “checkpoint inhibitor” is meant to refer to any small molecule chemical compound, antibody, nucleic acid molecule, or polypeptide, or fragments thereof, which inhibits the inhibitory pathways, allowing more extensive immune activity. In certain embodiments, the checkpoint inhibitor is an inhibitor of the programmed death-1 (PD-1) pathway, for example an anti-PD1 antibody, such as, but not limited to Nivolumab. In other embodiments, the checkpoint inhibitor is an anti-cytotoxic T-lymphocyte-associated antigen (CTLA-4) antibody. In additional embodiments, the checkpoint inhibitor is targeted at another member of the CD28CTLA4 Ig superfamily such as BTLA, LAG3. ICOS, PDL1 or KIR Page et al., Annual Review of Medicine 65:27 (2014)). In further additional embodiments, the checkpoint inhibitor is targeted at a member of the TNFR superfamily such as CD40, OX40, CD137, GITR, CD27 or TIM-3. In certain embodiments targeting a checkpoint inhibitor is accomplished with an inhibitory antibody or similar molecule. In other cases, it is accomplished with an agonist for the target; examples of this class include the stimulatory targets OX40 and GITR. In some cases it is accomplished with modulators targeting one or more of, e.g., chemotactic (CXCL12, CCL19) and immune modulating genes (PD-L2), and/or complement molecules provided in FIG. 4B.

The term “depth (coverage)” as used herein refers to the number of times a nucleotide is read during the sequencing process. Depth can be calculated from the length of the original genome (G), the number of reads (N), and the average read length (L) as N×L/G. For example, a hypothetical genome with 2,000 base pairs reconstructed from 8 reads with an average length of 500 nucleotides will have 2× redundancy. This parameter also enables one to estimate other quantities, such as the percentage of the genome covered by reads (sometimes also called coverage). A high coverage in shotgun sequencing is desired because it can overcome errors in base calling and assembly. The subject of DNA sequencing theory addresses the relationships of such quantities. Even though the sequencing accuracy for each individual nucleotide is very high, the very large number of nucleotides in the genome means that if an individual genome is only sequenced once, there will be a significant number of sequencing errors. Furthermore rare single-nucleotide polymorphisms (SNPs) are common. Hence to distinguish between sequencing errors and true SNPs, it is necessary to increase the sequencing accuracy even further by sequencing individual genomes a large number of times.

The term “deep sequencing” as used herein indicates that the total number of reads is many times larger than the length of the sequence under study. The term “deep” as used herein refers to a wide range of depths greater than or equal to 1× up to 100×.

The terms “complement,” “complement system” and “complement components” as used herein refer to proteins and protein fragments, including serum proteins, serosal proteins, and cell membrane receptors that are part of any of the classical complement pathway, the alternative complement pathway, and the lectin pathway. The terms “complement,” “complement system” and “complement components” also includes the defense molecules (protection molecules) CD46, CD55 and CD59.

The classical pathway is triggered by activation of the C1-complex. The C1-complex is composed of 1 molecule of C1q, 2 molecules of C r and 2 molecules of C1s, or C1qr2s2. This occurs when C1q binds to IgM or IgG complexed with antigens. A single pentameric IgM can initiate the pathway, while several, ideally six, IgGs are needed. This also occurs when C1q binds directly to the surface of the pathogen. Such binding leads to conformational changes in the C1q molecule, which leads to the activation of two C1r molecules. C1r is a serine protease. They then cleave C1s (another serine protease). The C1r2s2 component now splits C4 and then C2, producing C4a, C4b, C2a, and C2b. C4b and C2a bind to form the classical pathway C3-convertase (C4b2a complex), which promotes cleavage of C3 into C3a and C3b; C3b later joins with C4b2a (the C3 convertase) to make C5 convertase (C4b2a3b complex). The inhibition of C1r and C1s is controlled by C1-inhibitor (SERPING1).

The alternative pathway is continuously activated at a low level as a result of spontaneous C3 hydrolysis due to the breakdown of the internal thioester bond. The alternative pathway does not rely on pathogen-binding antibodies like the other pathways. C3b that is generated from C3 by a C3 convertase enzyme complex in the fluid phase is rapidly inactivated by factor H and factor I, as is the C3b-like C3 that is the product of spontaneous cleavage of the internal thioester. In contrast, when the internal thioester of C3 reacts with a hydroxyl or amino group of a molecule on the surface of a cell or pathogen, the C3b that is now covalently bound to the surface is protected from factor H-mediated inactivation. The surface-bound C3b may now bind factor B to form C3bB. This complex in the presence of factor D will be cleaved into Ba and Bb. Bb will remain associated with C3b to form C3bBb, which is the alternative pathway C3 convertase.

The C3bBb complex is stabilized by binding oligomers of factor P (Properdin). The stabilized C3 convertase. C3bBbP, then acts enzymatically to cleave much more C3, some of which becomes covalently attached to the same surface as C3b. This newly bound C3b recruits more B. D and P activity and greatly amplifies the complement activation. When complement is activated on a cell surface, the activation is limited by endogenous complement regulatory proteins, which include CD35, CD46, CD55 and CD59, depending on the cell. Pathogens, in general, don't have complement regulatory proteins Thus, the alternative complement pathway is able to distinguish self from non-self on the basis of the surface expression of complement regulatory proteins. Host cells don't accumulate cell surface C3b (and the proteolytic fragment of C3b called iC3b) because this is prevented by the complement regulatory proteins, while foreign cells, pathogens and abnormal surfaces may be heavily decorated with C3b and iC3b. Accordingly, the alternative complement pathway is one element of innate immunity.

Once the alternative C3 convertase enzyme is formed on a pathogen or cell surface, it may bind covalently another C3b, to form C3bBbC3bP, the C5 convertase. This enzyme then cleaves C5 to C5a, a potent anaphylatoxin, and C5b. The C5b then recruits and assembles C6, C7, C8 and multiple C9 molecules to assemble the membrane attack complex. This creates a hole or pore in the membrane that can kill or damage the pathogen or cell.

The lectin pathway is homologous to the classical pathway, but with the opsonin, mannose-binding lectin (MBL), and ficolins, instead of C1q. This pathway is activated by binding of MBL to mannose residues on the pathogen surface, which activates the MBL-associated serine proteases, MASP-1, and MASP-2 (very similar to C1r and C1s, respectively), which can then split C4 into C4a and C4b and C2 into C2a and C2b. C4b and C2a then bind together to form the classical C3-convertase, as in the classical pathway. Ficolins are homologous to MBL and function via MASP in a similar way. Several single-nucleotide polymorphisms have been described in M-ficolin in humans, with effect on ligand-binding ability and serum levels. Historically, the larger fragment of C2 was named C2a, but it is now referred as C2b. In invertebrates without an adaptive immune system, ficolins are expanded and their binding specificities diversified to compensate for the lack of pathogen-specific recognition molecules.

The term “MDSC” (myeloid-derived suppressor cells) refers to a heterogenous group of immune cells from the myeloid lineage (a family of cells that originate from bone marrow stem cells), to which dendritic cells, macrophages and neutrophils also belong. MDSCs strongly expand in pathological situations such as chronic infections and cancer, as a result of an altered hematopoiesis. Thus, it is yet unclear whether MDSCs represent a group of immature myeloid cell types that have stopped their differentiation towards DCs, macrophages or granulocytes, or if they represent a myeloid lineage apart. MDSCs are however discriminated from other myeloid cell types in which they possess strong immunosuppressive activities rather than immunostimulatory properties. Similarly to other myeloid cells, MDSCs interact with other immune cell types including T cells (the effector immune cells that kill pathogens, infected and cancer cells), dendritic cells, macrophages and NK cells to regulate their functions. Their mechanisms of action are beginning to be understood although they are still under heated debate and close examination by the scientific community. Nevertheless, clinical and experimental evidence has shown that cancer tissues with high infiltration of MDSC are associated with poor patient prognosis and resistance to therapies.

These signatures are useful in methods of monitoring a cancer in a subject by detecting a level of expression, activity and/or function of one or more signature genes or one or more products of one or more signature genes at a first time point, detecting a level of expression, activity and/or function of one or more signature genes or one or more products of one or more signature genes at a second time point, and comparing the first detected level of expression, activity and/or function with the second detected level of expression, activity and/or function, wherein a change in the first and second detected levels indicates a change in the cancer in the subject.

One unique aspect of the invention is the ability to relate expression of one gene or a gene signature in one cell type to that of another gene or signature in another cell type in the same tumor. In one embodiment, the methods and signatures of the invention are useful in patients with complex cancers, heterogeneous cancers or more than one cancer.

In an embodiment of the invention, these signatures are useful in monitoring subjects undergoing treatments and therapies for cancer to determine efficaciousness of the treatment or therapy. In an embodiment of the invention, these signatures are useful in monitoring subjects undergoing treatments and therapies for cancer to determine whether the patient is responsive to the treatment or therapy. In an embodiment of the invention, these signatures are also useful for selecting or modifying therapies and treatments that would be efficacious in treating, delaying the progression of or otherwise ameliorating a symptom of cancer. In an embodiment of the invention, the signatures provided herein are used for selecting a group of patients at a specific state of a disease with accuracy that facilitates selection of treatments.

The present invention also comprises a kit with a detection reagent that binds to one or more signature nucleic acids. Also provided by the invention is an array of detection reagents, e.g., oligonucleotides that can bind to one or more signature nucleic acids. Suitable detection reagents include nucleic acids that specifically identify one or more signature nucleic acids by having homologous nucleic acid sequences, such as oligonucleotide sequences, complementary to a portion of the signature nucleic acids packaged together in the form of a kit. The oligonucleotides can be fragments of the signature genes. For example the oligonucleotides can be 200, 150, 100, 50, 25, 10 or fewer nucleotides in length. The kit may contain in separate container or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions (e.g., written, tape, VCR. CD-ROM, etc.) for carrying out the assay may be included in the kit. The assay may for example be in the form of a Northern hybridization or DNA chips or a sandwich ELISA or any other method as known in the art. Alternatively, the kit contains a nucleic acid substrate array comprising one or more nucleic acid sequences.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company. Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the invention, which include a T cell modulating agent, targeted therapies and checkpoint inhibitors, are used to treat or alleviate a symptom associated with a cancer. The present invention also provides methods of treating or alleviating a symptom associated with cancer. A therapeutic regimen is carried out by identifying a subject, e.g., a human patient suffering from cancer, using standard methods.

Efficaciousness of treatment is determined in association with any known method for diagnosing or treating the particular cancer. The invention comprehends a treatment method or Drug Discovery method or method of formulating or preparing a treatment comprising any one of the methods or uses herein discussed.

The phrase “therapeutically effective amount” as used herein refers to a nontoxic but sufficient amount of a drug, agent, or compound to provide a desired therapeutic effect.

As used herein “patient” refers to any human being receiving or who may receive medical treatment.

A “polymorphic site” refers to a polynucleotide that differs from another polynucleotide by one or more single nucleotide changes.

A “somatic mutation” refers to a change in the genetic structure that is not inherited from a parent, and also not passed to offspring.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing a cancer (e.g., a person who is genetically predisposed) may receive prophylactic treatment to inhibit or delay symptoms of the disease.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999. Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for oral, rectal, intravenous, intramuscular, subcutaneous, inhalation, nasal, topical or transdermal, vaginal, or ophthalmic administration. Thus, the medicament may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, or aerosols.

In order to determine the genotype of a patient according to the methods of the present invention, it may be necessary to obtain a sample of genomic DNA from that patient. That sample of genomic DNA may be obtained from a sample of tissue or cells taken from that patient.

The tissue sample may comprise but is not limited to hair (including roots), skin, buccal swabs, blood, or saliva. The tissue sample may be marked with an identifying number or other indicia that relates the sample to the individual patient from which the sample was taken. The identity of the sample advantageously remains constant throughout the methods of the invention thereby guaranteeing the integrity and continuity of the sample during extraction and analysis. Alternatively, the indicia may be changed in a regular fashion that ensures that the data, and any other associated data, can be related back to the patient from whom the data was obtained. The amount/size of sample required is known to those skilled in the art.

Generally, the tissue sample may be placed in a container that is labeled using a numbering system bearing a code corresponding to the patient. Accordingly, the genotype of a particular patient is easily traceable.

In one embodiment of the invention, a sampling device and/or container may be supplied to the physician. The sampling device advantageously takes a consistent and reproducible sample from individual patients while simultaneously avoiding any cross-contamination of tissue. Accordingly, the size and volume of sample tissues derived from individual patients would be consistent.

According to the present invention, a sample of DNA is obtained from the tissue sample of the patient of interest. Whatever source of cells or tissue is used, a sufficient amount of cells must be obtained to provide a sufficient amount of DNA for analysis. This amount will be known or readily determinable by those skilled in the art.

DNA is isolated from the tissue/cells by techniques known to those skilled in the art (see, e.g., U.S. Pat. Nos. 6,548,256 and 5,989,431, Hirota et al., Jinrui Idengaku Zasshi. September 1989; 34(3):217-23 and John et al., Nucleic Acids Res. Jan. 25, 1991; 19(2):408; the disclosures of which are incorporated by reference in their entireties). For example, high molecular weight DNA may be purified from cells or tissue using proteinase K extraction and ethanol precipitation. DNA may be extracted from a patient specimen using any other suitable methods known in the art.

In certain embodiments, the invention involves a high-throughput single-cell RNA-Seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard, technology of U.S. provisional patent application Ser. No. 62/048,227 filed Sep. 9, 2014, the disclosure of which is incorporated by reference, may be used in or as to the invention. A combination of molecular barcoding and emulsion-based microfluidics to isolate, lyse, barcode, and prepare nucleic acids from individual cells in high-throughput is used. Microfluidic devices (for example, fabricated in polydimethylsiloxane), sub-nanoliter reverse emulsion droplets. These droplets are used to co-encapsulate nucleic acids with a barcoded capture bead. Each bead, for example, is uniquely barcoded so that each drop and its contents are distinguishable. The nucleic acids may come from any source known in the art, such as for example, those which come from a single cell, a pair of cells, a cellular lysate, or a solution. The cell is lysed as it is encapsulated in the droplet. To load single cells and barcoded beads into these droplets with Poisson statistics, 100,000 to 10 million such beads are needed to barcode ˜10,000-100,000 cells. In this regard there can be a single-cell sequencing library which may comprise: merging one uniquely barcoded mRNA capture microbead with a single-cell in an emulsion droplet having a diameter of 75-125 μm; lysing the cell to make its RNA accessible for capturing by hybridization onto RNA capture microbead; performing a reverse transcription either inside or outside the emulsion droplet to convert the cell's mRNA to a first strand cDNA that is covalently linked to the mRNA capture microbead; pooling the cDNA-attached microbeads from all cells: and preparing and sequencing a single composite RNA-Seq library. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; Zheng, et al., 2016, “Haplotyping germline and cancer genomes with high-throughput linked-read sequencing” Nature Biotechnology 34, 303-311; and International patent publication number WO 2014210353 A2, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology 33, 102-106.

Accordingly, it is envisioned as to or in the practice of the invention provides that there can be a method for preparing uniquely barcoded mRNA capture microbeads, which has a unique barcode and diameter suitable for microfluidic devices which may comprise: 1) performing reverse phosphoramidite synthesis on the surface of the bead in a pool-and-split fashion, such that in each cycle of synthesis the beads are split into four reactions with one of the four canonical nucleotides (T, C. G, or A) or unique oligonucleotides of length two or more bases; 2) repeating this process a large number of times, at least six, and optimally more than twelve, such that, in the latter, there are more than 16 million unique barcodes on the surface of each bead in the pool. (See www.ncbi.nlm.nih.gov/pmc/articles/PMC206447).

Likewise, in or as to the instant invention there can be an apparatus for creating a single-cell sequencing library via a microfluidic system, which may comprise: an oil-surfactant inlet which may comprise a filter and a carrier fluid channel, wherein said carrier fluid channel further may comprise a resistor; an inlet for an analyte which may comprise a filter and a carrier fluid channel, wherein said carrier fluid channel may further comprise a resistor; an inlet for mRNA capture microbeads and lysis reagent which may comprise a filter and a carrier fluid channel, wherein said carrier fluid channel may further comprise a resistor; said carrier fluid channels have a carrier fluid flowing therein at an adjustable or predetermined flow rate; wherein each said carrier fluid channels merge at a junction; and said junction being connected to a mixer, which contains an outlet for drops. Similarly, as to or in the practice of the instant invention there can be a method for creating a single-cell sequencing library which may comprise: merging one uniquely barcoded RNA capture microbead with a single-cell in an emulsion droplet having a diameter of 125 μm lysing the cell thereby capturing the RNA on the RNA capture microbead; performing a reverse transcription either after breakage of the droplets and collection of the microbeads; or inside the emulsion droplet to convert the cell's RNA to a first strand cDNA that is covalently linked to the RNA capture microbead; pooling the cDNA-attached microbeads from all cells; and preparing and sequencing a single composite RNA-Seq library; and, the emulsion droplet can be between 50-210 μm. In a further embodiment, the method wherein the diameter of the mRNA capture microbeads is from 10 μm to 95 μm. Thus, the practice of the instant invention comprehends preparing uniquely barcoded mRNA capture microbeads, which has a unique barcode and diameter suitable for microfluidic devices which may comprise: 1) performing reverse phosphoramidite synthesis on the surface of the bead in a pool-and-split fashion, such that in each cycle of synthesis the beads are split into four reactions with one of the four canonical nucleotides (T, C, G. or A); 2) repeating this process a large number of times, at least six, and optimally more than twelve, such that, in the latter, there are more than 16 million unique barcodes on the surface of each bead in the pool. The covalent bond can be polyethylene glycol. The diameter of the mRNA capture microbeads can be from 10 μm to 95 μm. Accordingly, it is also envisioned as to or in the practice of the invention that there can be a method for preparing uniquely barcoded mRNA capture microbeads, which has a unique barcode and diameter suitable for microfluidic devices which may comprise: 1) performing reverse phosphoramidite synthesis on the surface of the bead in a pool-and-split fashion, such that in each cycle of synthesis the beads are split into four reactions with one of the four canonical nucleotides (T, C, G, or A); 2) repeating this process a large number of times, at least six, and optimally more than twelve, such that, in the latter, there are more than 16 million unique barcodes on the surface of each bead in the pool. And, the diameter of the mRNA capture microbeads can be from 10 μm to 95 μm. Further, as to in the practice of the invention there can be an apparatus for creating a composite single-cell sequencing library via a microfluidic system, which may comprise: an oil-surfactant inlet which may comprise a filter and two carrier fluid channels, wherein said carrier fluid channel further may comprise a resistor; an inlet for an analyte which may comprise a filter and two carrier fluid channels, wherein said carrier fluid channel further may comprise a resistor; an inlet for mRNA capture microbeads and lysis reagent which may comprise a carrier fluid channel; said carrier fluid channels have a carrier fluid flowing therein at an adjustable and predetermined flow rate; wherein each said carrier fluid channels merge at a junction; and said junction being connected to a constriction for droplet pinch-off followed by a mixer, which connects to an outlet for drops. The analyte may comprise a chemical reagent, a genetically perturbed cell, a protein, a drug, an antibody, an enzyme, a nucleic acid, an organelle like the mitochondrion or nucleus, a cell or any combination thereof. In an embodiment of the apparatus the analyte is a cell. In a further embodiment the cell is a brain cell. In an embodiment of the apparatus the lysis reagent may comprise an anionic surfactant such as sodium lauroyl sarcosinate, or a chaotropic salt such as guanidinium thiocyanate. The filter can involve square PDMS posts; e.g., with the filter on the cell channel of such posts with sides ranging between 125-135 μm with a separation of 70-100 mm between the posts. The filter on the oil-surfactant inlet may comprise square posts of two sizes: one with sides ranging between 75-100 μm and a separation of 25-30 μm between them and the other with sides ranging between 40-50 μm and a separation of 10-15 μm. The apparatus can involve a resistor, e.g., a resistor that is serpentine having a length of 7000-9000 μm, width of 50-75 μm and depth of 100-150 mm. The apparatus can have channels having a length of 8000-12,000 μm for oil-surfactant inlet, 5000-7000 for analyte (cell) inlet, and 900-1200 μm for the inlet for microbead and lysis agent; and/or all channels having a width of 125-250 mm, and depth of 100-150 mm. The width of the cell channel can be 125-250 μm and the depth 100-150 μm. The apparatus can include a mixer having a length of 7000-9000 μm, and a width of 110-140 μm with 35-45o zig-zigs every 150 μm. The width of the mixer can be about 125 μm. The oil-surfactant can be a PEG Block Polymer, such as BIORAD™ QX200 Droplet Generation Oil. The carrier fluid can be a water-glycerol mixture.

In the practice of the invention or as to the invention, a mixture may comprise a plurality of microbeads adorned with combinations of the following elements: bead-specific oligonucleotide barcodes; additional oligonucleotide barcode sequences which vary among the oligonucleotides on an individual bead and can therefore be used to differentiate or help identify those individual oligonucleotide molecules; additional oligonucleotide sequences that create substrates for downstream molecular-biological reactions, such as oligo-dT (for reverse transcription of mature mRNAs), specific sequences (for capturing specific portions of the transcriptome, or priming for DNA polymerases and similar enzymes), or random sequences (for priming throughout the transcriptome or genome). The individual oligonucleotide molecules on the surface of any individual microbead may contain all three of these elements, and the third element may include both oligo-dT and a primer sequence. A mixture may comprise a plurality of microbeads, wherein said microbeads may comprise the following elements: at least one bead-specific oligonucleotide barcode; at least one additional identifier oligonucleotide barcode sequence, which varies among the oligonucleotides on an individual bead, and thereby assisting in the identification and of the bead specific oligonucleotide molecules; optionally at least one additional oligonucleotide sequences, which provide substrates for downstream molecular-biological reactions. A mixture may comprise at least one oligonucleotide sequence(s), which provide for substrates for downstream molecular-biological reactions. In a further embodiment the downstream molecular biological reactions are for reverse transcription of mature mRNAs; capturing specific portions of the transcriptome, priming for DNA polymerases and/or similar enzymes; or priming throughout the transcriptome or genome. The mixture may involve additional oligonucleotide sequence(s) which may comprise an oligo-dT sequence. The mixture further may comprise the additional oligonucleotide sequence which may comprise a primer sequence. The mixture may further comprise the additional oligonucleotide sequence which may comprise an oligo-dT sequence and a primer sequence.

Examples of the labeling substance which may be employed include labeling substances known to those skilled in the art, such as fluorescent dyes, enzymes, coenzymes, chemiluminescent substances, and radioactive substances. Specific examples include radioisotopes (e.g., 32P, 14C, 125I, 3H, and 131I), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added. Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3.5 disulfonate; N-(4-anilino-1-naphthyl)maleimide; anthranilamide; BODIPY; Brilliant Yellow: coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocvanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine: pararosaniline; Phenol Red: B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives: Cy3; Cy5; Cy5.5; Cy7; IRD 700; IRD 800; La Jolta Blue; phthalo cyanine; and naphthalo cyanine. A fluorescent label may be a fluorescent protein, such as blue fluorescent protein, cyan fluorescent protein, green fluorescent protein, red fluorescent protein, yellow fluorescent protein or any photoconvertible protein. Colorimetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code. Advantageously, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation. Advantageously, agents may be uniquely labeled in a dynamic manner (see, e.g., US provisional patent application Ser. No. 61/703,884 filed Sep. 21, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached. Oligonucleotide tags may be detectable by virtue of their nucleotide sequence, or by virtue of a non-nucleic acid detectable moiety that is attached to the oligonucleotide such as but not limited to a fluorophore, or by virtue of a combination of their nucleotide sequence and the non-nucleic acid detectable moiety. A detectable oligonucleotide tag may comprise one or more non-oligonucleotide detectable moieties. Examples of detectable moieties may include, but are not limited to, fluorophores, microparticles including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci. USA 97(17):9461-9466, 2000), biotin, DNP (dinitrophenyl), fucose, digoxigenin, haptens, and other detectable moieties known to those skilled in the art. In some embodiments, the detectable moieties may be quantum dots. Methods for detecting such moieties are described herein and/or are known in the art. Thus, detectable oligonucleotide tags may be, but are not limited to, oligonucleotides which may comprise unique nucleotide sequences, oligonucleotides which may comprise detectable moieties, and oligonucleotides which may comprise both unique nucleotide sequences and detectable moieties. A unique label may be produced by sequentially attaching two or more detectable oligonucleotide tags to each other. The detectable tags may be present or provided in a plurality of detectable tags. The same or a different plurality of tags may be used as the source of each detectable tag may be part of a unique label. In other words, a plurality of tags may be subdivided into subsets and single subsets may be used as the source for each tag. One or more other species may be associated with the tags. In particular, nucleic acids released by a lysed cell may be ligated to one or more tags. These may include, for example, chromosomal DNA, RNA transcripts, tRNA, mRNA, mitochondrial DNA, or the like. Such nucleic acids may be sequenced, in addition to sequencing the tags themselves, which may yield information about the nucleic acid profile of the cells, which can be associated with the tags, or the conditions that the corresponding droplet or cell was exposed to.

The invention accordingly may involve or be practiced as to high throughput and high resolution delivery of reagents to individual emulsion droplets that may contain cells, organelles, nucleic acids, proteins, etc. through the use of monodisperse aqueous droplets that are generated by a microfluidic device as a water-in-oil emulsion. The droplets are carried in a flowing oil phase and stabilized by a surfactant. In one aspect single cells or single organelles or single molecules (proteins, RNA, DNA) are encapsulated into uniform droplets from an aqueous solution/dispersion. In a related aspect, multiple cells or multiple molecules may take the place of single cells or single molecules. The aqueous droplets of volume ranging from 1 pL to 10 nL work as individual reactors. 104 to 105 single cells in droplets may be processed and analyzed in a single run. To utilize microdroplets for rapid large-scale chemical screening or complex biological library identification, different species of microdroplets, each containing the specific chemical compounds or biological probes cells or molecular barcodes of interest, have to be generated and combined at the preferred conditions, e.g., mixing ratio, concentration, and order of combination. Each species of droplet is introduced at a confluence point in a main microfluidic channel from separate inlet microfluidic channels. Preferably, droplet volumes are chosen by design such that one species is larger than others and moves at a different speed, usually slower than the other species, in the carrier fluid, as disclosed in U.S. Publication No. US 2007/0195127 and International Publication No. WO 2007/089541, each of which are incorporated herein by reference in their entirety. The channel width and length is selected such that faster species of droplets catch up to the slowest species. Size constraints of the channel prevent the faster moving droplets from passing the slower moving droplets resulting in a train of droplets entering a merge zone. Multi-step chemical reactions, biochemical reactions, or assay detection chemistries often require a fixed reaction time before species of different type are added to a reaction. Multi-step reactions are achieved by repeating the process multiple times with a second, third or more confluence points each with a separate merge point. Highly efficient and precise reactions and analysis of reactions are achieved when the frequencies of droplets from the inlet channels are matched to an optimized ratio and the volumes of the species are matched to provide optimized reaction conditions in the combined droplets. Fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc. In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled. In one arrangement, the expansion and/or contraction of liquid reservoirs may be used to steer or sort a fluidic droplet into a channel, e.g., by causing directed movement of the liquid containing the fluidic droplet. In another, the expansion and/or contraction of the liquid reservoir may be combined with other flow-controlling devices and methods, e.g., as described herein. Non-limiting examples of devices able to cause the expansion and/or contraction of a liquid reservoir include pistons. Key elements for using microfluidic channels to process droplets include: (1) producing droplet of the correct volume, (2) producing droplets at the correct frequency and (3) bringing together a first stream of sample droplets with a second stream of sample droplets in such a way that the frequency of the first stream of sample droplets matches the frequency of the second stream of sample droplets. Preferably, bringing together a stream of sample droplets with a stream of premade library droplets in such a way that the frequency of the library droplets matches the frequency of the sample droplets. Methods for producing droplets of a uniform volume at a regular frequency are well known in the art. One method is to generate droplets using hydrodynamic focusing of a dispersed phase fluid and immiscible carrier fluid, such as disclosed in U.S. Publication No. US 2005/0172476 and International Publication No. WO 2004/002627. It is desirable for one of the species introduced at the confluence to be a pre-made library of droplets where the library contains a plurality of reaction conditions, e.g., a library may contain plurality of different compounds at a range of concentrations encapsulated as separate library elements for screening their effect on cells or enzymes, alternatively a library could be composed of a plurality of different primer pairs encapsulated as different library elements for targeted amplification of a collection of loci, alternatively a library could contain a plurality of different antibody species encapsulated as different library elements to perform a plurality of binding assays. The introduction of a library of reaction conditions onto a substrate is achieved by pushing a premade collection of library droplets out of a vial with a drive fluid. The drive fluid is a continuous fluid. The drive fluid may comprise the same substance as the carrier fluid (e.g., a fluorocarbon oil). For example, if a library consists of ten pico-liter droplets is driven into an inlet channel on a microfluidic substrate with a drive fluid at a rate of 10,000 pico-liters per second, then nominally the frequency at which the droplets are expected to enter the confluence point is 1000 per second. However, in practice droplets pack with oil between them that slowly drains. Over time the carrier fluid drains from the library droplets and the number density of the droplets (number/mL) increases. Hence, a simple fixed rate of infusion for the drive fluid does not provide a uniform rate of introduction of the droplets into the microfluidic channel in the substrate. Moreover, library-to-library variations in the mean library droplet volume result in a shift in the frequency of droplet introduction at the confluence point. Thus, the lack of uniformity of droplets that results from sample variation and oil drainage provides another problem to be solved. For example if the nominal droplet volume is expected to be 10 pico-liters in the library, but varies from 9 to 11 pico-liters from library-to-library then a 10.000 pico-liter/second infusion rate will nominally produce a range in frequencies from 900 to 1,100 droplet per second. In short, sample to sample variation in the composition of dispersed phase for droplets made on chip, a tendency for the number density of library droplets to increase over time and library-to-library variations in mean droplet volume severely limit the extent to which frequencies of droplets may be reliably matched at a confluence by simply using fixed infusion rates. In addition, these limitations also have an impact on the extent to which volumes may be reproducibly combined. Combined with typical variations in pump flow rate precision and variations in channel dimensions, systems are severely limited without a means to compensate on a run-to-run basis. The foregoing facts not only illustrate a problem to be solved, but also demonstrate a need for a method of instantaneous regulation of microfluidic control over microdroplets within a microfluidic channel. Combinations of surfactant(s) and oils must be developed to facilitate generation, storage, and manipulation of droplets to maintain the unique chemical/biochemical/biological environment within each droplet of a diverse library. Therefore, the surfactant and oil combination must (1) stabilize droplets against uncontrolled coalescence during the drop forming process and subsequent collection and storage, (2) minimize transport of any droplet contents to the oil phase and/or between droplets, and (3) maintain chemical and biological inertness with contents of each droplet (e.g., no adsorption or reaction of encapsulated contents at the oil-water interface, and no adverse effects on biological or chemical constituents in the droplets). In addition to the requirements on the droplet library function and stability, the surfactant-in-oil solution must be coupled with the fluid physics and materials associated with the platform. Specifically, the oil solution must not swell, dissolve, or degrade the materials used to construct the microfluidic chip, and the physical properties of the oil (e.g., viscosity, boiling point, etc.) must be suited for the flow and operating conditions of the platform. Droplets formed in oil without surfactant are not stable to permit coalescence, so surfactants must be dissolved in the oil that is used as the continuous phase for the emulsion library. Surfactant molecules are amphiphilic—part of the molecule is oil soluble, and part of the molecule is water soluble. When a water-oil interface is formed at the nozzle of a microfluidic chip for example in the inlet module described herein, surfactant molecules that are dissolved in the oil phase adsorb to the interface. The hydrophilic portion of the molecule resides inside the droplet and the fluorophilic portion of the molecule decorates the exterior of the droplet. The surface tension of a droplet is reduced when the interface is populated with surfactant, so the stability of an emulsion is improved. In addition to stabilizing the droplets against coalescence, the surfactant should be inert to the contents of each droplet and the surfactant should not promote transport of encapsulated components to the oil or other droplets. A droplet library may be made up of a number of library elements that are pooled together in a single collection (see, e.g., US Patent Publication No. 2010002241). Libraries may vary in complexity from a single library element to 1015 library elements or more. Each library element may be one or more given components at a fixed concentration. The element may be, but is not limited to, cells, organelles, virus, bacteria, yeast, beads, amino acids, proteins, polypeptides, nucleic acids, polynucleotides or small molecule chemical compounds. The element may contain an identifier such as a label. The terms “droplet library” or “droplet libraries” are also referred to herein as an “emulsion library” or “emulsion libraries.” These terms are used interchangeably throughout the specification. A cell library element may include, but is not limited to, hybridomas, B-cells, primary cells, cultured cell lines, cancer cells, stem cells, cells obtained from tissue, or any other cell type. Cellular library elements are prepared by encapsulating a number of cells from one to hundreds of thousands in individual droplets. The number of cells encapsulated is usually given by Poisson statistics from the number density of cells and volume of the droplet. However, in some cases the number deviates from Poisson statistics as described in Edd et al., “Controlled encapsulation of single-cells into monodisperse picolitre drops.” Lab Chip, 8(8): 1262-1264, 2008. The discrete nature of cells allows for libraries to be prepared in mass with a plurality of cellular variants all present in a single starting media and then that media is broken up into individual droplet capsules that contain at most one cell. These individual droplets capsules are then combined or pooled to form a library consisting of unique library elements. Cell division subsequent to, or in some embodiments following, encapsulation produces a clonal library element. A bead based library element may contain one or more beads, of a given type and may also contain other reagents, such as antibodies, enzymes or other proteins. In the case where all library elements contain different types of beads, but the same surrounding media the library elements may all be prepared from a single starting fluid or have a variety of starting fluids. In the case of cellular libraries prepared in mass from a collection of variants, such as genomically modified, yeast or bacteria cells, the library elements will be prepared from a variety of starting fluids. Often it is desirable to have exactly one cell per droplet with only a few droplets containing more than one cell when starting with a plurality of cells or yeast or bacteria, engineered to produce variants on a protein. In some cases, variations from Poisson statistics may be achieved to provide an enhanced loading of droplets such that there are more droplets with exactly one cell per droplet and few exceptions of empty droplets or droplets containing more than one cell. Examples of droplet libraries are collections of droplets that have different contents, ranging from beads, cells, small molecules, DNA, primers, antibodies. Smaller droplets may be in the order of femtoliter (fL) volume drops, which are especially contemplated with the droplet dispensors. The volume may range from about 5 to about 600 fL. The larger droplets range in size from roughly 0.5 micron to 500 micron in diameter, which corresponds to about 1 pico liter to 1 nano liter. However, droplets may be as small as 5 microns and as large as 500 microns. Preferably, the droplets are at less than 100 microns, about 1 micron to about 100 microns in diameter. The most preferred size is about 20 to 40 microns in diameter (10 to 100 picoliters). The preferred properties examined of droplet libraries include osmotic pressure balance, uniform size, and size ranges. The droplets within the emulsion libraries of the present invention may be contained within an immiscible oil, which may comprise at least one fluorosurfactant. In some embodiments, the fluorosurfactant within the immiscible fluorocarbon oil may be a block copolymer consisting of one or more perfluorinated polyether (PFPE) blocks and one or more polyethylene glycol (PEG) blocks. In other embodiments, the fluorosurfactant is a triblock copolymer consisting of a PEG center block covalently bound to two PFPE blocks by amide linking groups. The presence of the fluorosurfactant (similar to uniform size of the droplets in the library) is critical to maintain the stability and integrity of the droplets and is also essential for the subsequent use of the droplets within the library for the various biological and chemical assays described herein. Fluids (e.g., aqueous fluids, immiscible oils, etc.) and other surfactants that may be utilized in the droplet libraries of the present invention are described in greater detail herein. The present invention can accordingly involve an emulsion library which may comprise a plurality of aqueous droplets within an immiscible oil (e.g., fluorocarbon oil) which may comprise at least one fluorosurfactant, wherein each droplet is uniform in size and may comprise the same aqueous fluid and may comprise a different library element. The present invention also provides a method for forming the emulsion library which may comprise providing a single aqueous fluid which may comprise different library elements, encapsulating each library element into an aqueous droplet within an immiscible fluorocarbon oil that may comprise at least one fluorosurfactant, wherein each droplet is uniform in size and may comprise the same aqueous fluid and may comprise a different library element, and pooling the aqueous droplets within an immiscible fluorocarbon oil which may comprise at least one fluorosurfactant, thereby forming an emulsion library. For example, in one type of emulsion library, all different types of elements (e.g., cells or beads), may be pooled in a single source contained in the same medium. After the initial pooling, the cells or beads are then encapsulated in droplets to generate a library of droplets wherein each droplet with a different type of bead or cell is a different library element. The dilution of the initial solution enables the encapsulation process. In some embodiments, the droplets formed will either contain a single cell or bead or will not contain anything. i.e., be empty. In other embodiments, the droplets formed will contain multiple copies of a library element. The cells or beads being encapsulated are generally variants on the same type of cell or bead. In another example, the emulsion library may comprise a plurality of aqueous droplets within an immiscible fluorocarbon oil, wherein a single molecule may be encapsulated, such that there is a single molecule contained within a droplet for every 20-60 droplets produced (e.g., 20, 25, 30, 35, 40, 45, 50, 55, 60 droplets, or any integer in between). Single molecules may be encapsulated by diluting the solution containing the molecules to such a low concentration that the encapsulation of single molecules is enabled. In one specific example, a LacZ plasmid DNA was encapsulated at a concentration of 20 fM after two hours of incubation such that there was about one gene in 40 droplets, where 10 μm droplets were made at 10 kHz per second. Formation of these libraries rely on limiting dilutions.

The present invention also provides an emulsion library which may comprise at least a first aqueous droplet and at least a second aqueous droplet within a fluorocarbon oil that may comprise at least one fluorosurfactant, wherein the at least first and the at least second droplets are uniform in size and comprise a different aqueous fluid and a different library element. The present invention also provides a method for forming the emulsion library which may comprise providing at least a first aqueous fluid which may comprise at least a first library of elements, providing at least a second aqueous fluid which may comprise at least a second library of elements, encapsulating each element of said at least first library into at least a first aqueous droplet within an immiscible fluorocarbon oil which may comprise at least one fluorosurfactant, encapsulating each element of said at least second library into at least a second aqueous droplet within an immiscible fluorocarbon oil which may comprise at least one fluorosurfactant, wherein the at least first and the at least second droplets are uniform in size and may comprise a different aqueous fluid and a different library element, and pooling the at least first aqueous droplet and the at least second aqueous droplet within an immiscible fluorocarbon oil which may comprise at least one fluorosurfactant thereby forming an emulsion library. One of skill in the art will recognize that methods and systems of the invention are not preferably practiced as to cells, mutations, etc., as herein disclosed, but that the invention need not be limited to any particular type of sample, and methods and systems of the invention may be used with any type of organic, inorganic, or biological molecule (see, e.g., U.S. Patent Publication No. 20120122714). In particular embodiments the sample may include nucleic acid target molecules. Nucleic acid molecules may be synthetic or derived from naturally occurring sources. In one embodiment, nucleic acid molecules may be isolated from a biological sample containing a variety of other components, such as proteins, lipids and non-template nucleic acids. Nucleic acid target molecules may be obtained from any cellular material, obtained from an animal, plant, bacterium, fungus, or any other cellular organism. In certain embodiments, the nucleic acid target molecules may be obtained from a single cell. Biological samples for use in the present invention may include viral particles or preparations. Nucleic acid target molecules may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid target molecules may also be isolated from cultured cells, such as a primary cell culture or a cell line. The cells or tissues from which target nucleic acids are obtained may be infected with a virus or other intracellular pathogen. A sample may also be total RNA extracted from a biological specimen, a cDNA library, viral, or genomic DNA. Generally, nucleic acid may be extracted from a biological sample by a variety of techniques such as those described by Maniatis, et al., Molecular Cloning: A Laboratory Manual, Cold Spring Harbor, N.Y., pp. 280-281 (1982). Nucleic acid molecules may be single-stranded, double-stranded, or double-stranded with single-stranded regions (for example, stem- and loop-structures). Nucleic acid obtained from biological samples typically may be fragmented to produce suitable fragments for analysis. Target nucleic acids may be fragmented or sheared to desired length, using a variety of mechanical, chemical and/or enzymatic methods. DNA may be randomly sheared via sonication, e.g., Covaris method, brief exposure to a DNase, or using a mixture of one or more restriction enzymes, or a transposase or nicking enzyme. RNA may be fragmented by brief exposure to an RNase, heat plus magnesium, or by shearing. The RNA may be converted to cDNA. If fragmentation is employed, the RNA may be converted to cDNA before or after fragmentation. In one embodiment, nucleic acid from a biological sample is fragmented by sonication. In another embodiment, nucleic acid is fragmented by a hydroshear instrument. Generally, individual nucleic acid target molecules may be from about 40 bases to about 40 kb. Nucleic acid molecules may be single-stranded, double-stranded, or double-stranded with single-stranded regions (for example, stem- and loop-structures). A biological sample as described herein may be homogenized or fractionated in the presence of a detergent or surfactant. The concentration of the detergent in the buffer may be about 0.05% to about 10.0%. The concentration of the detergent may be up to an amount where the detergent remains soluble in the solution. In one embodiment, the concentration of the detergent is between 0.1% to about 2%. The detergent, particularly a mild one that is non-denaturing, may act to solubilize the sample. Detergents may be ionic or nonionic. Examples of nonionic detergents include triton, such as the Triton™ X series (Triton™ X-100 t-Oct-C6H4-(OCH2-CH2)xOH, x=9-10, Triton™ X-100R, Triton™ X-114 x=7-8), octyl glucoside, polyoxyethylene(9)dodecyl ether, digitonin, IGEPAL™ CA630 octylphenyl polyethylene glycol, n-octyl-beta-D-glucopyranoside (betaOG), n-dodecyl-beta. Tween™. 20 polyethylene glycol sorbitan monolaurate, Tween™ 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14E06), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant. Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid. Size selection of the nucleic acids may be performed to remove very short fragments or very long fragments. The nucleic acid fragments may be partitioned into fractions which may comprise a desired number of fragments using any suitable method known in the art. Suitable methods to limit the fragment size in each fragment are known in the art. In various embodiments of the invention, the fragment size is limited to between about 10 and about 100 Kb or longer. A sample in or as to the instant invention may include individual target proteins, protein complexes, proteins with translational modifications, and protein/nucleic acid complexes. Protein targets include peptides, and also include enzymes, hormones, structural components such as viral capsid proteins, and antibodies. Protein targets may be synthetic or derived from naturally-occurring sources. The invention protein targets may be isolated from biological samples containing a variety of other components including lipids, non-template nucleic acids, and nucleic acids. Protein targets may be obtained from an animal, bacterium, fungus, cellular organism, and single cells. Protein targets may be obtained directly from an organism or from a biological sample obtained from the organism, including bodily fluids such as blood, urine, cerebrospinal fluid, seminal fluid, saliva, sputum, stool and tissue. Protein targets may also be obtained from cell and tissue lysates and biochemical fractions. An individual protein is an isolated polypeptide chain. A protein complex includes two or polypeptide chains. Samples may include proteins with post translational modifications including but not limited to phosphorylation, methionine oxidation, deamidation, glycosylation, ubiquitination, carbamoylation, s-carboxymethylation, acetylation, and methylation. Protein/nucleic acid complexes include cross-linked or stable protein-nucleic acid complexes. Extraction or isolation of individual proteins, protein complexes, proteins with translational modifications, and protein/nucleic acid complexes is performed using methods known in the art.

The invention can thus involve forming sample droplets. The droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803). Anderson et al. (U.S. Pat. No. 7,041,481 and which reissued as RE41.780) and European publication number EP2047910 to Raindance Technologies Inc. The content of each of which is incorporated by reference herein in its entirety. The present invention may relates to systems and methods for manipulating droplets within a high throughput microfluidic system. A microfluid droplet encapsulates a differentiated cell. The cell is lysed and its mRNA is hybridized onto a capture bead containing barcoded oligo dT primers on the surface, all inside the droplet. The barcode is covalently attached to the capture bead via a flexible multi-atom linker like PEG. In a preferred embodiment, the droplets are broken by addition of a fluorosurfactant (like perfluorooctanol), washed, and collected. A reverse transcription (RT) reaction is then performed to convert each cell's mRNA into a first strand cDNA that is both uniquely barcoded and covalently linked to the mRNA capture bead. Subsequently, a universal primer via a template switching reaction is amended using conventional library preparation protocols to prepare an RNA-Seq library. Since all of the mRNA from any given cell is uniquely barcoded, a single library is sequenced and then computationally resolved to determine which mRNAs came from which cells. In this way, through a single sequencing run, tens of thousands (or more) of distinguishable transcriptomes can be simultaneously obtained. The oligonucleotide sequence may be generated on the bead surface. During these cycles, beads were removed from the synthesis column, pooled, and aliquoted into four equal portions by mass; these bead aliquots were then placed in a separate synthesis column and reacted with either dG, dC, dT, or dA phosphoramidite. In other instances, dinucleotide, trinucleotides, or oligonucleotides that are greater in length are used, in other instances, the oligo-dT tail is replaced by gene specific oligonucleotides to prime specific targets (singular or plural), random sequences of any length for the capture of all or specific RNAs. This process was repeated 12 times for a total of 4¹²=16,777,216 unique barcode sequences. Upon completion of these cycles, 8 cycles of degenerate oligonucleotide synthesis were performed on all the beads, followed by 30 cycles of dT addition. In other embodiments, the degenerate synthesis is omitted, shortened (less than 8 cycles), or extended (more than 8 cycles); in others, the 30 cycles of dT addition are replaced with gene specific primers (single target or many targets) or a degenerate sequence. The aforementioned microfluidic system is regarded as the reagent delivery system microfluidic library printer or droplet library printing system of the present invention. Droplets are formed as sample fluid flows from droplet generator which contains lysis reagent and barcodes through microfluidic outlet channel which contains oil, towards junction. Defined volumes of loaded reagent emulsion, corresponding to defined numbers of droplets, are dispensed on-demand into the flow stream of carrier fluid. The sample fluid may typically comprise an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. Any liquid or buffer that is physiologically compatible with nucleic acid molecules can be used. The carrier fluid may include one that is immiscible with the sample fluid. The carrier fluid can be a non-polar solvent, decane (e.g., tetradecane or hexadecane), fluorocarbon oil, silicone oil, an inert oil such as hydrocarbon, or another oil (for example, mineral oil). The carrier fluid may contain one or more additives, such as agents which reduce surface tensions (surfactants). Surfactants can include Tween, Span, fluorosurfactants, and other agents that are soluble in oil relative to water. In some applications, performance is improved by adding a second surfactant to the sample fluid. Surfactants can aid in controlling or optimizing droplet size, flow and uniformity, for example by reducing the shear force needed to extrude or inject droplets into an intersecting channel. This can affect droplet volume and periodicity, or the rate or frequency at which droplets break off into an intersecting channel. Furthermore, the surfactant can serve to stabilize aqueous emulsions in fluorinated oils from coalescing. Droplets may be surrounded by a surfactant which stabilizes the droplets by reducing the surface tension at the aqueous oil interface. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglyceryl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates). In some cases, an apparatus for creating a single-cell sequencing library via a microfluidic system provides for volume-driven flow, wherein constant volumes are injected over time. The pressure in fluidic channels is a function of injection rate and channel dimensions. In one embodiment, the device provides an oil/surfactant inlet; an inlet for an analyte; a filter, an inlet for mRNA capture microbeads and lysis reagent; a carrier fluid channel which connects the inlets; a resistor; a constriction for droplet pinch-off; a mixer; and an outlet for drops. In an embodiment the invention provides apparatus for creating a single-cell sequencing library via a microfluidic system, which may comprise: an oil-surfactant inlet which may comprise a filter and a carrier fluid channel, wherein said carrier fluid channel may further comprise a resistor; an inlet for an analyte which may comprise a filter and a carrier fluid channel, wherein said carrier fluid channel may further comprise a resistor; an inlet for mRNA capture microbeads and lysis reagent which may comprise a filter and a carrier fluid channel, wherein said carrier fluid channel further may comprise a resistor; said carrier fluid channels have a carrier fluid flowing therein at an adjustable or predetermined flow rate; wherein each said carrier fluid channels merge at a junction; and said junction being connected to a mixer, which contains an outlet for drops. Accordingly, an apparatus for creating a single-cell sequencing library via a microfluidic system or microfluidic flow scheme for single-cell RNA-seq is envisioned. Two channels, one carrying cell suspensions, and the other carrying uniquely barcoded mRNA capture bead, lysis buffer and library preparation reagents meet at a junction and is immediately co-encapsulated in an inert carrier oil, at the rate of one cell and one bead per drop. In each drop, using the bead's barcode tagged oligonucleotides as cDNA template, each mRNA is tagged with a unique, cell-specific identifier. The invention also encompasses use of a Drop-Seq library of a mixture of mouse and human cells. The carrier fluid may be caused to flow through the outlet channel so that the surfactant in the carrier fluid coats the channel walls. The fluorosurfactant can be prepared by reacting the perfluorinated polyether DuPont Krytox 157 FSL, FSM, or FSH with aqueous ammonium hydroxide in a volatile fluorinated solvent. The solvent and residual water and ammonia can be removed with a rotary evaporator. The surfactant can then be dissolved (e.g., 2.5 wt %) in a fluorinated oil (e.g., Fluorinert (3M)), which then serves as the carrier fluid. Activation of sample fluid reservoirs to produce regent droplets is based on the concept of dynamic reagent delivery (e.g., combinatorial barcoding) via an on demand capability. The on demand feature may be provided by one of a variety of technical capabilities for releasing delivery droplets to a primary droplet, as described herein. From this disclosure and herein cited documents and knowledge in the art, it is within the ambit of the skilled person to develop flow rates, channel lengths, and channel geometries; and establish droplets containing random or specified reagent combinations can be generated on demand and merged with the “reaction chamber” droplets containing the samples/cells/substrates of interest. By incorporating a plurality of unique tags into the additional droplets and joining the tags to a solid support designed to be specific to the primary droplet, the conditions that the primary droplet is exposed to may be encoded and recorded. For example, nucleic acid tags can be sequentially ligated to create a sequence reflecting conditions and order of same. Alternatively, the tags can be added independently appended to solid support. Non-limiting examples of a dynamic labeling system that may be used to bioinformatically record information can be found at U.S. Provisional Patent Application entitled “Compositions and Methods for Unique Labeling of Agents” filed Sep. 21, 2012 and Nov. 29, 2012. In this way, two or more droplets may be exposed to a variety of different conditions, where each time a droplet is exposed to a condition, a nucleic acid encoding the condition is added to the droplet each ligated together or to a unique solid support associated with the droplet such that, even if the droplets with different histories are later combined, the conditions of each of the droplets are remain available through the different nucleic acids. Non-limiting examples of methods to evaluate response to exposure to a plurality of conditions can be found at U.S. Provisional Patent Application entitled “Systems and Methods for Droplet Tagging” filed Sep. 21, 2012. Accordingly, in or as to the invention it is envisioned that there can be the dynamic generation of molecular barcodes (e.g., DNA oligonucleotides, fluorophores, etc.) either independent from or in concert with the controlled delivery of various compounds of interest (drugs, small molecules, siRNA, CRISPR guide RNAs, reagents, etc.). For example, unique molecular barcodes can be created in one array of nozzles while individual compounds or combinations of compounds can be generated by another nozzle array. Barcodes/compounds of interest can then be merged with cell-containing droplets. An electronic record in the form of a computer log file is kept to associate the barcode delivered with the downstream reagent(s) delivered. This methodology makes it possible to efficiently screen a large population of cells for applications such as single-cell drug screening, controlled perturbation of regulatory pathways, etc. The device and techniques of the disclosed invention facilitate efforts to perform studies that require data resolution at the single cell (or single molecule) level and in a cost effective manner. The invention envisions a high throughput and high resolution delivery of reagents to individual emulsion droplets that may contain cells, nucleic acids, proteins, etc. through the use of monodisperse aqueous droplets that are generated one by one in a microfluidic chip as a water-in-oil emulsion. Being able to dynamically track individual cells and droplet treatments/combinations during life cycle experiments, and having an ability to create a library of emulsion droplets on demand with the further capability of manipulating the droplets through the disclosed process(es) are advantageous. In the practice of the invention there can be dynamic tracking of the droplets and create a history of droplet deployment and application in a single cell based environment. Droplet generation and deployment is produced via a dynamic indexing strategy and in a controlled fashion in accordance with disclosed embodiments of the present invention. Microdroplets can be processed, analyzed and sorted at a highly efficient rate of several thousand droplets per second, providing a powerful platform which allows rapid screening of millions of distinct compounds, biological probes, proteins or cells either in cellular models of biological mechanisms of disease, or in biochemical, or pharmacological assays. A plurality of biological assays as well as biological synthesis are contemplated. Polymerase chain reactions (PCR) are contemplated (see, e.g., US Patent Publication No. 20120219947). Methods of the invention may be used for merging sample fluids for conducting any type of chemical reaction or any type of biological assay. There may be merging sample fluids for conducting an amplification reaction in a droplet. Amplification refers to production of additional copies of a nucleic acid sequence and is generally carried out using polymerase chain reaction or other technologies well known in the art (e.g., Dieffenbach and Dveksler, PCR Primer, a Laboratory Manual, Cold Spring Harbor Press, Plainview, N.Y. [1995]). The amplification reaction may be any amplification reaction known in the art that amplifies nucleic acid molecules, such as polymerase chain reaction, nested polymerase chain reaction, polymerase chain reaction-single strand conformation polymorphism, ligase chain reaction (Barany F. (1991) PNAS 88:189-193; Barany F. (1991) PCR Methods and Applications 1:5-16), ligase detection reaction (Barany F. (1991) PNAS 88:189-193), strand displacement amplification and restriction fragments length polymorphism, transcription based amplification system, nucleic acid sequence-based amplification, rolling circle amplification, and hyper-branched rolling circle amplification. In certain embodiments, the amplification reaction is the polymerase chain reaction. Polymerase chain reaction (PCR) refers to methods by K. B. Mullis (U.S. Pat. Nos. 4,683,195 and 4,683,202, hereby incorporated by reference) for increasing concentration of a segment of a target sequence in a mixture of genomic DNA without cloning or purification. The process for amplifying the target sequence includes introducing an excess of oligonucleotide primers to a DNA mixture containing a desired target sequence, followed by a precise sequence of thermal cycling in the presence of a DNA polymerase. The primers are complementary to their respective strands of the double stranded target sequence. To effect amplification, primers are annealed to their complementary sequence within the target molecule. Following annealing, the primers are extended with a polymerase so as to form a new pair of complementary strands. The steps of denaturation, primer annealing and polymerase extension may be repeated many times (i.e., denaturation, annealing and extension constitute one cycle; there may be numerous cycles) to obtain a high concentration of an amplified segment of a desired target sequence. The length of the amplified segment of the desired target sequence is determined by relative positions of the primers with respect to each other, and therefore, this length is a controllable parameter. Methods for performing PCR in droplets are shown for example in Link et al. (U.S. Patent application numbers 2008/0014589, 2008/0003142, and 2010/0137163), Anderson et al. (U.S. Pat. No. 7,041,481 and which reissued as RE41,780) and European publication number EP2047910 to Raindance Technologies Inc. The content of each of which is incorporated by reference herein in its entirety. The first sample fluid contains nucleic acid templates. Droplets of the first sample fluid are formed as described above. Those droplets will include the nucleic acid templates. In certain embodiments, the droplets will include only a single nucleic acid template, and thus digital PCR may be conducted. The second sample fluid contains reagents for the PCR reaction. Such reagents generally include Taq polymerase, deoxynucleotides of type A, C, G and T, magnesium chloride, and forward and reverse primers, all suspended within an aqueous buffer. The second fluid also includes detectably labeled probes for detection of the amplified target nucleic acid, the details of which are discussed below. This type of partitioning of the reagents between the two sample fluids is not the only possibility. In some instances, the first sample fluid will include some or all of the reagents necessary for the PCR whereas the second sample fluid will contain the balance of the reagents necessary for the PCR together with the detection probes. Primers may be prepared by a variety of methods including but not limited to cloning of appropriate sequences and direct chemical synthesis using methods well known in the art (Narang et al., Methods Enzymol., 68:90 (1979); Brown et al., Methods Enzymol., 68:109 (1979)). Primers may also be obtained from commercial sources such as Operon Technologies, Amersham Pharmacia Biotech, Sigma, and Life Technologies. The primers may have an identical melting temperature. The lengths of the primers may be extended or shortened at the 5′ end or the 3′ end to produce primers with desired melting temperatures. Also, the annealing position of each primer pair may be designed such that the sequence and, length of the primer pairs yield the desired melting temperature. The simplest equation for determining the melting temperature of primers smaller than 25 base pairs is the Wallace Rule (Td=2(A+ T)+4(G+C)). Computer programs may also be used to design primers, including but not limited to Array Designer Software (Arrayit Inc.), Oligonucleotide Probe Sequence Design Software for Genetic Analysis (Olympus Optical Co.), NetPrimer, and DNAsis from Hitachi Software Engineering. The TM (melting or annealing temperature) of each primer is calculated using software programs such as Oligo Design, available from Invitrogen Corp.

A droplet containing the nucleic acid is then caused to merge with the PCR reagents in the second fluid according to methods of the invention described above, producing a droplet that includes Taq polymerase, deoxynucleotides of type A, C, G and T, magnesium chloride, forward and reverse primers, detectably labeled probes, and the target nucleic acid. Once mixed droplets have been produced, the droplets are thermal cycled, resulting in amplification of the target nucleic acid in each droplet. Droplets may be flowed through a channel in a serpentine path between heating and cooling lines to amplify the nucleic acid in the droplet. The width and depth of the channel may be adjusted to set the residence time at each temperature, which may be controlled to anywhere between less than a second and minutes. The three temperature zones may be used for the amplification reaction. The three temperature zones are controlled to result in denaturation of double stranded nucleic acid (high temperature zone), annealing of primers (low temperature zones), and amplification of single stranded nucleic acid to produce double stranded nucleic acids (intermediate temperature zones). The temperatures within these zones fall within ranges well known in the art for conducting PCR reactions. See for example, Sambrook et al. (Molecular Cloning, A Laboratory Manual, 3rd edition, Cold Spring Harbor Laboratory Press, Cold Spring Harbor. N.Y., 2001). The three temperature zones can be controlled to have temperatures as follows: 95° C. (TH), 55° C. (TL), 72° C. (TM). The prepared sample droplets flow through the channel at a controlled rate. The sample droplets first pass the initial denaturation zone (TH) before thermal cycling. The initial preheat is an extended zone to ensure that nucleic acids within the sample droplet have denatured successfully before thermal cycling. The requirement for a preheat zone and the length of denaturation time required is dependent on the chemistry being used in the reaction. The samples pass into the high temperature zone, of approximately 95° C., where the sample is first separated into single stranded DNA in a process called denaturation. The sample then flows to the low temperature, of approximately 55° C., where the hybridization process takes place, during which the primers anneal to the complementary sequences of the sample. Finally, as the sample flows through the third medium temperature, of approximately 72° C., the polymerase process occurs when the primers are extended along the single strand of DNA with a thermostable enzyme. The nucleic acids undergo the same thermal cycling and chemical reaction as the droplets pass through each thermal cycle as they flow through the channel. The total number of cycles in the device is easily altered by an extension of thermal zones. The sample undergoes the same thermal cycling and chemical reaction as it passes through N amplification cycles of the complete thermal device. In other aspects, the temperature zones are controlled to achieve two individual temperature zones for a PCR reaction. In certain embodiments, the two temperature zones are controlled to have temperatures as follows: 95° C. (TH) and 60° C. (TL). The sample droplet optionally flows through an initial preheat zone before entering thermal cycling. The preheat zone may be important for some chemistry for activation and also to ensure that double stranded nucleic acid in the droplets is fully denatured before the thermal cycling reaction begins. In an exemplary embodiment, the preheat dwell length results in approximately 10 minutes preheat of the droplets at the higher temperature. The sample droplet continues into the high temperature zone, of approximately 95° C., where the sample is first separated into single stranded DNA in a process called denaturation. The sample then flows through the device to the low temperature zone, of approximately 60° C., where the hybridization process takes place, during which the primers anneal to the complementary sequences of the sample. Finally the polymerase process occurs when the primers are extended along the single strand of DNA with a thermostable enzyme. The sample undergoes the same thermal cycling and chemical reaction as it passes through each thermal cycle of the complete device. The total number of cycles in the device is easily altered by an extension of block length and tubing. After amplification, droplets may be flowed to a detection module for detection of amplification products. The droplets may be individually analyzed and detected using any methods known in the art, such as detecting for the presence or amount of a reporter. Generally, a detection module is in communication with one or more detection apparatuses. Detection apparatuses may be optical or electrical detectors or combinations thereof. Examples of suitable detection apparatuses include optical waveguides, microscopes, diodes, light stimulating devices, (e.g., lasers), photo multiplier tubes, and processors (e.g., computers and software), and combinations thereof, which cooperate to detect a signal representative of a characteristic, marker, or reporter, and to determine and direct the measurement or the sorting action at a sorting module. Further description of detection modules and methods of detecting amplification products in droplets are shown in Link et al. (U.S. patent application numbers 2008/0014589, 2008/0003142, and 2010/0137163) and European publication number EP2047910 to Raindance Technologies Inc.

Examples of assays are also ELISA assays (see, e.g., US Patent Publication No. 20100022414). The present invention provides another emulsion library which may comprise a plurality of aqueous droplets within an immiscible fluorocarbon oil which may comprise at least one fluorosurfactant, wherein each droplet is uniform in size and may comprise at least a first antibody, and a single element linked to at least a second antibody, wherein said first and second antibodies are different. In one example, each library element may comprise a different bead, wherein each bead is attached to a number of antibodies and the bead is encapsulated within a droplet that contains a different antibody in solution. These antibodies may then be allowed to form “ELISA sandwiches,” which may be washed and prepared for a ELISA assay. Further, these contents of the droplets may be altered to be specific for the antibody contained therein to maximize the results of the assay. Single-cell assays are also contemplated as part of the present invention (see, e.g., Ryan et al., Biomicrofluidics 5, 021501 (2011) for an overview of applications of microfluidics to assay individual cells). A single-cell assay may be contemplated as an experiment that quantifies a function or property of an individual cell when the interactions of that cell with its environment may be controlled precisely or may be isolated from the function or property under examination. The research and development of single-cell assays is largely predicated on the notion that genetic variation causes disease and that small subpopulations of cells represent the origin of the disease. Methods of assaying compounds secreted from cells, subcellular components, cell-cell or cell-drug interactions as well as methods of patterning individual cells are also contemplated within the present invention.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application. Ser. No. 14/104,990), US 2014-0234972 A1 (U.S. application Ser. No. 14/183,471), US 2014-0227787 A1 (U.S. application Ser. No. 14/256,912). US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application. Ser. No. 14/104,837) and US 2014-0179006 A1 (U.S. application Ser. No. 14/183,486), US 2014-0170753 (U.S. application Ser. No. 14/183,429); European Patents EP 2 784 162 B1 and EP 2 771 468 B1; European Patent Applications EP 2 771 468 (EP13818570.7), EP 2 764 103 (EP13824232.6), and EP 2 784 162 (EP14170383.5); and PCT Patent Publications PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO2014/093701 (PCT/US2013/074800), WO2014/018423 (PCT/US2013/051418). WO 2014/204723 (PCT/US2014/041790), WO 2014/204724 (PCT/US2014/041800), WO 2014/204725 (PCT/US2014/041803). WO 2014/204726 (PCT/US2014/041804), WO 2014/204727 (PCT/US2014/041806). WO 2014/204728 (PCT/US2014/041808), WO 2014/204729 (PCT/US2014/041809). Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/836,127, 61/836,101, 61/836,080 and 61/835,973, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT Patent applications Nos: PCT/US2014/041803, PCT/US2014/041800, PCT/US2014/041809, PCT/US2014/041804 and PCT/US2014/041806, each filed Jun. 10, 2014 Jun. 10, 2014; PCT/US2014/041808 filed Jun. 11, 2014; and PCT/US2014/62558 filed Oct. 28, 2014, and U.S. Provisional Patent Applications Ser. Nos. 61/915,150, 61/915,301, 61/915,267 and 61/915,260, each filed Dec. 12, 2013; 61/757,972 and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. provisional patent applications Nos. 62/055,484, 62/055,460, and 62/055,487, filed Sep. 25, 2014; U.S. provisional patent application 61/980,012, filed Apr. 15, 2014; and U.S. provisional patent application 61/939,242 filed Feb. 12, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. Reference is made to US provisional patent application U.S. Ser. No. 61/980,012 filed Apr. 15, 2014. Reference is made to PCT application designating, inter alia. the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013.

Mention is also made of U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); US application 62/091,462, 12 Dec. 2014, DEAD GUIDES FOR CRISPR TRANSCRIPTION FACTORS; U.S. application 62/096,324, 23 Dec. 2014, DEAD GUIDES FOR CRISPR TRANSCRIPTION FACTORS; U.S. application 62/091,456, 12 Dec. 2014, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, RNA-TARGETING SYSTEM; U.S. application 62/096,656, 24 Dec. 2014, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 15, CELLULAR TARGETING FOR EXTRACELLULAR EXOSOMAL REPORTING; U.S. application 62/054,490, 24 Sep. 2014, DELIVERY. USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; US application 62/054,675, 24 Sep. 2014, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; US application 62/087,475, 4 Dec. 2014, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

Also with respect to general information on CRISPR-Cas Systems, mention is made of the following (also hereby incorporated herein by reference):
Multiplex genome engineering using CRISPR/Cas systems. Cong, L., Ran, F. A., Cox, D., Lin, S., Barretto, R, Habib, N., Hsu, P. D., Wu, X., Jiang, W., Marraffini, L. A., & Zhang, F. Science February 15; 339(6121):819-23 (2013):
RNA-guided editing of bacterial genomes using CRISPR-Cas systems. Jiang W., Bikard D., Cox D., Zhang F, Marraffini L A. Nat Biotechnol March; 31(3):233-9 (2013):
One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9:153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott. D A., Inoue. A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang. F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print];
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014):
CRISPR-Cas9 Knockin Mice for Genome Editing and Cancer Modeling. Platt R J, Chen S. Zhou Y. Yim M J, Swiech L, Kempton H R. Dahlman J E, Pamas O, Eisenhaure T M, Jovanovic M, Graham D B, Jhunjhunwala S. Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014):
Development and Applications of CRISPR-Cas9 for Genome Engineering, Hsu P D, Lander E S, Zhang F., Cell. June 5; 157(6):1262-78 (2014).
Genetic screens in human cells using the CRISPR/Cas9 system, Wang T. Wei J J, Sabatini D M, Lander E S., Science. January 3; 343(6166): 80-84. doi: 10.1126/science. 1246981 (2014);
Rational design of highly active sgRNAs for CRISPR-Cas9-mediated gene inactivation. Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12): 1262-7 (2014):
In vivo interrogation of gene function in the mammalian brain using CRISPR-Cas9. Swiech L, Heidenreich M. Banerjee A, Habib N. Li Y. Trombetta J, Sur M, Zhang F., (published online 19 Oct. 2014) Nat Biotechnol. January; 33(1): 102-6 (2015):
Genome-scale transcriptional activation by an engineered CRISPR-Cas9 complex, Konermann S, Brigham M D, Trevino A E, Joung J. Abudayyeh O O, Barcena C. Hsu P D, Habib N. Gootenberg J S, Nishimasu H, Nureki O, Zhang F., Nature. January 29; 517(7536):583-8 (2015).
A split-Cas9 architecture for inducible genome editing and transcription modulation. Zetsche B. Volz S E, Zhang F., (published online 2 Feb. 2015) Nat Biotechnol. February; 33(2): 139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9:520(7546): 186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Pamas et al., “A Genome-wide CRISPR Screen in Primary Immune Cells to Dissect Regulatory Networks.” Cell 162, 675-686 (Jul. 30, 2015).
Ramanan et al., CRISPR/Cas9 cleavage of viral DNA efficiently suppresses hepatitis B virus.” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
Zetsche et al., “Cpf1 Is a Single RNA-Guided Endonuclease of a Class 2 CRISPR-Cas System,” Cell 163, 1-13 (Oct. 22, 2015)
Shmakov et al., “Discovery and Functional Characterization of Diverse Class 2 CRISPR-Cas Systems,” Molecular Cell 60, 1-13 (Available online Oct. 22, 2015)
   each of which is incorporated herein by reference, may be considered in the practice of the instant invention, and discussed briefly below:
   Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
   Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
   Wang et al. (2013) used the CRISPR/Cas system for the one-step generation of mice carrying mutations in multiple genes which were traditionally generated in multiple steps by sequential recombination in embryonic stem cells and/or time-consuming intercrossing of mice with a single mutation. The CRISPR/Cas system will greatly accelerate the in vivo study of functionally redundant genes and of epistatic gene interactions.
   Konermann et al. (2013) addressed the need in the art for versatile and robust technologies that enable optical and chemical modulation of DNA-binding domains based CRISPR Cas9 enzyme and also Transcriptional Activator Like Effectors
   Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
   Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.
   Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
   Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
   Nishimasu et al. reported the crystal structure of Streptococcus pyogenes Cas9 in complex with sgRNA and its target DNA at 2.5 A^{o} resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
   Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
   Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
   Hsu et al. (2014) is a review article that discusses generally CRISPR-Cas9 history from yogurt to genome editing, including genetic screening of cells.
   Wang et al. (2014) relates to a pooled, loss-of-function genetic screening approach suitable for both positive and negative selection that uses a genome-scale lentiviral single guide RNA (sgRNA) library.
   Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
   Swiech et al. demonstrate that AAV-mediated SpCas9 genome editing can enable reverse genetic studies of gene function in the brain.
   Konermann et al. (2015) discusses the ability to attach multiple effector domains, e.g., transcriptional activator, functional and epigenomic regulators at appropriate positions on the guide such as stem or tetraloop with and without linkers.
   Zetsche et al. demonstrates that the Cas9 enzyme can be split into two and hence the assembly of Cas9 for activation can be controlled.
   Chen et al. relates to multiplex screening by demonstrating that a genome-wide in vivo CRISPR-Cas9 screen in mice reveals genes regulating lung metastasis.
   Ran et al. (2015) relates to SaCas9 and its ability to edit genomes and demonstrates that one cannot extrapolate from biochemical assays. Shalem et al. (2015) described ways in which catalytically inactive Cas9 (dCas9) fusions are used to synthetically repress (CRISPRi) or activate (CRISPRa) expression, showing, advances using Cas9 for genome-scale screens, including arrayed and pooled screens, knockout approaches that inactivate genomic loci and strategies that modulate transcriptional activity.
   Shalem et al. (2015) described ways in which catalytically inactive Cas9 (dCas9) fusions are used to synthetically repress (CRISPRi) or activate (CRISPRa) expression, showing, advances using Cas9 for genome-scale screens, including arrayed and pooled screens, knockout approaches that inactivate genomic loci and strategies that modulate transcriptional activity.
   Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR/Cas9 knockout.
   Pamas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
   Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
   Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
   Zetsche et al. (2015) reported the characterization of Cpf1, a putative class 2 CRISPR effector. It was demonstrated that Cpf1 mediates robust DNA interference with features distinct from Cas9. Identifying this mechanism of interference broadens our understanding of CRISPR-Cas systems and advances their genome editing applications.
   Shmakov et al. (2015) reported the characterization of three distinct Class 2 CRISPR-Cas systems. The effectors of two of the identified systems, C2c1 and C2c3, contain RuvC like endonuclease domains distantly related to Cpf1. The third system, C2c2, contains an effector with two predicted HEPN RNase domains.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

In addition, mention is made of PCT application PCT/US4/70057, Attorney Reference 47627.99.2060 and BI-2013/107 entitled “DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS (claiming priority from one or more or all of US provisional patent applications: 62/054,490, filed Sep. 24, 2014; 62/010,441, filed Jun. 10, 2014; and 61/915,118, 61/915,215 and 61/915,148, each filed on Dec. 12, 2013) (“the Particle Delivery PCT”), incorporated herein by reference, with respect to a method of preparing an sgRNA-and-Cas9 protein containing particle comprising admixing a mixture comprising an sgRNA and Cas9 protein (and optionally HDR template) with a mixture comprising or consisting essentially of or consisting of surfactant, phospholipid, biodegradable polymer, lipoprotein and alcohol; and particles from such a process. For example, wherein Cas9 protein and sgRNA were mixed together at a suitable, e.g., 3:1 to 1:3 or 2:1 to 1:2 or 1:1 molar ratio, at a suitable temperature, e.g., 15-30C, e.g., 20-25C, e.g., room temperature, for a suitable time, e.g., 15-45, such as 30 minutes, advantageously in sterile, nuclease free buffer, e.g., 1×PBS. Separately, particle components such as or comprising: a surfactant, e.g., cationic lipid, e.g., 1,2-dioleoyl-3-trimethylammonium-propane (DOTAP); phospholipid, e.g., dimyristoylphosphatidylcholine (DMPC); biodegradable polymer, such as an ethylene-glycol polymer or PEG, and a lipoprotein, such as a low-density lipoprotein. e.g., cholesterol were dissolved in an alcohol, advantageously a C1-6 alkyl alcohol, such as methanol, ethanol, isopropanol, e.g., 100% ethanol. The two solutions were mixed together to form particles containing the Cas9-sgRNA complexes. Accordingly, sgRNA may be pre-complexed with the Cas9 protein, before formulating the entire complex in a particle. Formulations may be made with a different molar ratio of different components known to promote delivery of nucleic acids into cells (e.g. 1,2-dioleoyl-3-trimethylammonium-propane (DOTAP), 1,2-ditetradecanoyl-sn-glycero-3-phosphocholine (DMPC), polyethylene glycol (PEG), and cholesterol) For example DOTAP:DMPC:PEG:Cholesterol Molar Ratios may be DOTAP 100, DMPC 0, PEG 0, Cholesterol 0; or DOTAP 90, DMPC 0, PEG 10, Cholesterol 0; or DOTAP 90, DMPC 0, PEG 5, Cholesterol 5. DOTAP 100, DMPC 0, PEG 0, Cholesterol 0. That application accordingly comprehends admixing sgRNA. Cas9 protein and components that form a particle; as well as particles from such admixing. Aspects of the instant invention can involve particles; for example, particles using a process analogous to that of the Particle Delivery PCT, e.g., by admixing a mixture comprising sgRNA and/or Cas9 as in the instant invention and components that form a particle, e.g., as in the Particle Delivery PCT, to form a particle and particles from such admixing (or, of course, other particles involving sgRNA and/or Cas9 as in the instant invention).

In general, the CRISPR-Cas or CRISPR system is as used in the foregoing documents, such as WO 2014/093622 (PCT/US2013/074667) and refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In embodiments of the invention the terms guide sequence and guide RNA, i.e. RNA capable of guiding Cas to a target genomic locus, are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW. Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In a classic CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99%6 or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87%6 or 86% or 85% or 846 or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In particularly preferred embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e. an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence.

The methods according to the invention as described herein comprehend inducing one or more mutations in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s) or sgRNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 40, 45, 50, 75, 100, 200, 300, 400 or 500 nucleotides at each target sequence of said cell(s) via the guide(s) RNA(s) or sgRNA(s).

For minimization of toxicity and off-target effect, it will be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D10A mutation) can be delivered with a pair of guide RNAs targeting a site of interest. Guide sequences and strategies to minimize toxicity and off-target effects can be as in WO 2014/093622 (PCT/US2013/074667), or, via mutation as herein.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

The nucleic acid molecule encoding a Cas is advantageously codon optimized Cas. An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a Cas is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way how the Cas transgene is introduced in the cell is may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus, such as for instance one or more oncogenic mutations, as for instance and without limitation described in Platt et al. (2014), Chen et al., (2014) or Kumar et al., (2009).

In some embodiments, the Cas sequence is fused to one or more nuclear localization sequences (NLSs), such as about or more than about 1, 2, 3, 4, 5, 6, 7, 8, 9, 10, or more NLSs. In some embodiments, the Cas comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In a preferred embodiment of the invention, the Cas comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 1); the NLS from nucleoplasmin (e.g. the nucleoplasmin bipartite NLS with the sequence KRPAATKKAGQAKKKK) (SEQ ID NO: 2); the c-myc NLS having the amino acid sequence PAAKRVKLD (SEQ ID NO: 3) or RQRRNELKRSP(SEQ ID NO: 4); the hRNPA1 M9 NLS having the sequence NQSSNFGPMKGGNFGGRSSGPYGGGGQYFAKPRNQGGY(SEQ ID NO: 5); the sequence RMRIZFKNKGKDTAELRRRRVEVSVELRKAKKDEQILKRRNV (SEQ ID NO: 6) of the IBB domain from importin-alpha; the sequences VSRKRPRP (SEQ ID NO: 7) and PPKKARED (SEQ ID NO: 8) of the myoma T protein; the sequence PQPKKKPL (SEQ ID NO: 9) of human p53; the sequence SALIKKKKKMAP (SEQ ID NO: 10) of mouse c-abl IV; the sequences DRLRR (SEQ ID NO: 11) and PKQKKRK (SEQ ID NO: 12) of the influenza virus NS1; the sequence RKLKKKIKKL (SEQ ID NO: 13) of the Hepatitis virus delta antigen; the sequence REKKKFLKRR (SEQ ID NO: 14) of the mouse Mx1 protein; the sequence KRKGDEVDGVDEVAKKKSKK (SEQ ID NO: 15) of the human poly(ADP-ribose) polymerase; and the sequence RKCLQAGMNLEARKTKK (SEQ ID NO: 16) of the steroid hormone receptors (human) glucocorticoid. In general, the one or more NLSs are of sufficient strength to drive accumulation of the Cas in a detectable amount in the nucleus of a eukaryotic cell. In general, strength of nuclear localization activity may derive from the number of NLSs in the Cas, the particular NLS(s) used, or a combination of these factors. Detection of accumulation in the nucleus may be performed by any suitable technique. For example, a detectable marker may be fused to the Cas, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or Cas enzyme activity), as compared to a control no exposed to the Cas or complex, or exposed to a Cas lacking the one or more NLSs.

In certain aspects the invention involves vectors, e.g. for delivering or introducing in a cell the DNA targeting agent according to the invention as described herein, such as by means of example Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid.” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s) (e.g., sgRNAs); and, when a single vector provides for more than 16 RNA(s) (e.g., sgRNAs), one or more promoter(s) can drive expression of more than one of the RNA(s) (e.g., sgRNAs), e.g., when there are 32 RNA(s) (e.g., sgRNAs), each promoter can drive expression of two RNA(s) (e.g., sgRNAs), and when there are 48 RNA(s) (e.g., sgRNAs), each promoter can drive expression of three RNA(s) (e.g., sgRNAs). By simple arithmetic and well established cloning protocols and the teachings in this disclosure one skilled in the art can readily practice the invention as to the RNA(s) (e.g., sgRNA(s) for a suitable exemplary vector such as AAV, and a suitable promoter such as the U6 promoter, e.g., U6-sgRNAs. For example, the packaging limit of AAV is ˜4.7 kb. The length of a single U6-sgRNA (plus restriction sites for cloning) is 361 bp. Therefore, the skilled person can readily fit about 12-16, e.g., 13 U6-sgRNA cassettes in a single vector. This can be assembled by any suitable means, such as a golden gate strategy used for TALE assembly (www.genome-engineering.org/taleffectors/). The skilled person can also use a tandem guide strategy to increase the number of U6-sgRNAs by approximately 1.5 times, e.g., to increase from 12-16, e.g., 13 to approximately 18-24, e.g., about 19 U6-sgRNAs. Therefore, one skilled in the art can readily reach approximately 18-24, e.g., about 19 promoter-RNAs, e.g., U6-sgRNAs in a single vector, e.g., an AAV vector. A further means for increasing the number of promoters and RNAs, e.g., sgRNA(s) in a vector is to use a single promoter (e.g., U6) to express an array of RNAs, e.g., sgRNAs separated by cleavable sequences. And an even further means for increasing the number of promoter-RNAs, e.g., sgRNAs in a vector, is to express an array of promoter-RNAs, e.g., sgRNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short,

www.nature.com/mt/journal/v16/n9/abs/mt2008144a.html). In an advantageous embodiment, AAV may package U6 tandem sgRNA targeting up to about 50 genes. Accordingly, from the knowledge in the art and the teachings in this disclosure the skilled person can readily make and use vector(s), e.g., a single vector, expressing multiple RNAs or guides or sgRNAs under the control or operatively or functionally linked to one or more promoters-especially as to the numbers of RNAs or guides or sgRNAs discussed herein, without any undue experimentation.

A poly nucleic acid sequence encoding the DNA targeting agent according to the invention as described herein, such as by means of example guide RNA(s), e.g., sgRNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Through this disclosure and the knowledge in the art, the DNA targeting agent as described herein, such as, TALEs, CRISPR-Cas systems, etc., or components thereof or nucleic acid molecules thereof (including, for instance HDR template) or nucleic acid molecules encoding or providing components thereof may be delivered by a delivery system herein described both generally and in detail.

Vector delivery, e.g., plasmid, viral delivery: By means of example, the CRISPR enzyme, for instance a Cas9, and/or any of the present RNAs, for instance a guide RNA, can be delivered using any suitable vector, e.g., plasmid or viral vectors, such as adeno associated virus (AAV), lentivirus, adenovirus or other viral vector types, or combinations thereof. The DNA targeting agent as described herein, such as Cas9 and one or more guide RNAs can be packaged into one or more vectors, e.g., plasmid or viral vectors. In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplar) ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10⁷ particles, more preferably at least about 1×10⁸ particles (e.g., about 1×10⁸-1×10¹¹ particles or about 1×10⁸-1×10¹² particles), and most preferably at least about 1×10⁰ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10¹⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In an embodiment herein the delivery is via a plasmid. In such plasmid compositions, the dosage should be a sufficient amount of plasmid to elicit a response. For instance, suitable quantities of plasmid DNA in plasmid compositions can be from about 0.1 to about 2 mg, or from about 1 μg to about 10 μg per 70 kg individual. Plasmids of the invention will generally comprise (i) a promoter; (ii) a sequence encoding a DNA targeting agent as described herein, such as a comprising a CRISPR enzyme, operably linked to said promoter; (iii) a selectable marker; (iv) an origin of replication; and (v) a transcription terminator downstream of and operably linked to (ii). The plasmid can also encode the RNA components of a CRISPR complex, but one or more of these may instead be encoded on a different vector.

The doses herein are based on an average 70 kg individual. The frequency of administration is within the ambit of the medical or veterinary practitioner (e.g., physician, veterinarian), or scientist skilled in the art. It is also noted that mice used in experiments are typically about 20 g and from mice experiments one can scale up to a 70 kg individual.

In some embodiments the RNA molecules of the invention are delivered in liposome or lipofectin formulations and the like and can be prepared by methods well known to those skilled in the art. Such methods are described, for example, in U.S. Pat. Nos. 5,593,972, 5,589,466, and 5,580,859, which are herein incorporated by reference. Delivery systems aimed specifically at the enhanced and improved delivery of siRNA into mammalian cells have been developed, (see, for example, Shen et al FEBS Let. 2003, 539:111-114; Xia et al., Nat. Biotech. 2002, 20:1006-1010; Reich et al., Mol. Vision. 2003, 9: 210-216; Sorensen et al., J. Mol. Biol. 2003, 327: 761-766; Lewis et al., Nat. Gen. 2002, 32: 107-108 and Simeoni et al., NAR 2003, 31, 11: 2717-2724) and may be applied to the present invention, siRNA has recently been successfully used for inhibition of gene expression in primates (see for example. Tolentino et al., Retina 24(4):660 which may also be applied to the present invention.

Indeed, RNA delivery is a useful method of in vivo delivery. It is possible to deliver the DNA targeting agent as described herein, such as Cas9 and gRNA (and, for instance, HR repair template) into cells using liposomes or particles. Thus delivery of the CRISPR enzyme, such as a Cas9 and/or delivery of the RNAs of the invention may be in RNA form and via microvesicles, liposomes or particles. For example, Cas9 mRNA and gRNA can be packaged into liposomal particles for delivery in vivo. Liposomal transfection reagents such as lipofectamine from Life Technologies and other reagents on the market can effectively deliver RNA molecules into the liver.

Means of delivery of RNA also preferred include delivery of RNA via nanoparticles (Cho, S., Goldberg, M., Son. S., Xu, Q., Yang, F., Mei, Y., Bogatyrev, S., Langer, R. and Anderson, D., Lipid-like nanoparticles for small interfering RNA delivery to endothelial cells, Advanced Functional Materials, 19: 3112-3118, 2010) or exosomes (Schroeder. A., Levins, C., Cortez, C., Langer, R., and Anderson, D., Lipid-based nanotherapeutics for siRNA delivery, Journal of Internal Medicine, 267: 9-21, 2010, PMID: 20059641). Indeed, exosomes have been shown to be particularly useful in delivery siRNA, a system with some parallels to the CRISPR system. For instance, El-Andaloussi S, et al. (“Exosome-mediated delivery of siRNA in vitro and in vivo.” Nat Protoc. 2012 December; 7(12):2112-26. doi: 10.1038/nprot.2012.131. Epub 2012 Nov. 15.) describe how exosomes are promising tools for drug delivery across different biological barriers and can be harnessed for delivery of siRNA in vitro and in vivo. Their approach is to generate targeted exosomes through transfection of an expression vector, comprising an exosomal protein fused with a peptide ligand. The exosomes are then purify and characterized from transfected cell supernatant, then RNA is loaded into the exosomes. Delivery or administration according to the invention can be performed with exosomes, in particular but not limited to the brain. Vitamin E (α-tocopherol) may be conjugated with CRISPR Cas and delivered to the brain along with high density lipoprotein (HDL), for example in a similar manner as was done by Uno et al. (HUMAN GENE THERAPY 22:711-719 (June 2011)) for delivering short-interfering RNA (siRNA) to the brain. Mice were infused via Osmotic minipumps (model 1007D; Alzet, Cupertino, Calif.) filled with phosphate-buffered saline (PBS) or free TocsiBACE or Toc-siBACE-IDL and connected with Brain Infusion Kit 3 (Alzet). A brain-infusion cannula was placed about 0.5 mm posterior to the bregma at midline for infusion into the dorsal third ventricle. Uno et al. found that as little as 3 nmol of Toc-siRNA with HDL could induce a target reduction in comparable degree by the same ICV infusion method. A similar dosage of CRISPR Cas conjugated to α-tocopherol and co-administered with HDL targeted to the brain may be contemplated for humans in the present invention, for example, about 3 nmol to about 3 μmol of CRISPR Cas targeted to the brain may be contemplated. Zou et al. ((HUMAN GENE THERAPY 22:465-475 (April 2011)) describes a method of lentiviral-mediated delivery of short-hairpin RNAs targeting PKCγ for in vivo gene silencing in the spinal cord of rats. Zou et al. administered about 10 μl of a recombinant lentivirus having a titer of 1×10⁹ transducing units (TU)/ml by an intrathecal catheter. A similar dosage of CRISPR Cas expressed in a lentiviral vector targeted to the brain may be contemplated for humans in the present invention, for example, about 10-50 ml of CRISPR Cas targeted to the brain in a lentivirus having a titer of 1×10⁹ transducing units (TU)/ml may be contemplated.

In terms of local delivery to the brain, this can be achieved in various ways. For instance, material can be delivered intrastriatally e.g. by injection. Injection can be performed stereotactically via a craniotomy.

Enhancing NHEJ or HR efficiency is also helpful for delivery. It is preferred that NHEJ efficiency is enhanced by co-expressing end-processing enzymes such as Trex2 (Dumitrache et al. Genetics. 2011 August; 188(4): 787-797). It is preferred that HR efficiency is increased by transiently inhibiting NHEJ machineries such as Ku70 and Ku86. HR efficiency can also be increased by co-expressing prokaryotic or eukaryotic homologous recombination enzymes such as RecBCD, RecA.

Packaging and Promoters Generally

Ways to package nucleic acid molecules, in particular the DNA targeting agent according to the invention as described herein, such as Cas9 coding nucleic acid molecules, e.g., DNA, into vectors, e.g., viral vectors, to mediate genome modification in vivo include:

To achieve NHEJ-mediated gene knockout:
Single virus vector:
   Vector containing two or more expression cassettes:
   Promoter-Cas9 coding nucleic acid molecule-terminator
   Promoter-gRNA1-terminator
   Promoter-gRNA2-terminator
   Promoter-gRNA(N)-terminator (up to size limit of vector)
Double virus vector:
   Vector 1 containing one expression cassette for driving the expression of Cas9
   Promoter-Cas9 coding nucleic acid molecule-terminator
   Vector 2 containing one more expression cassettes for driving the expression of one or more guideRNAs
   Promoter-gRNA1-terminator
   Promoter-gRNA(N)-terminator (up to size limit of vector)

To mediate homology-directed repair.
In addition to the single and double virus vector approaches described above, an additional vector is used to deliver a homology-direct repair template.

The promoter used to drive Cas9 coding nucleic acid molecule expression can include:

AAV ITR can serve as a promoter: this is advantageous for eliminating the need for an additional promoter element (which can take up space in the vector). The additional space freed up can be used to drive the expression of additional elements (gRNA, etc.). Also, ITR activity is relatively weaker, so can be used to reduce potential toxicity due to over expression of Cas9.

For ubiquitous expression, can use promoters: CMV, CAG, CBh, PGK, SV40, Ferritin heavy or light chains, etc.

For brain or other CNS expression, can use promoters: SynapsinI for all neurons, CaMKIIalpha for excitatory neurons. GAD67 or GAD65 or VGAT for GABAergic neurons, etc.

For liver expression, can use Albumin promoter.

For lung expression, can use SP-B.

For endothelial cells, can use ICAM.

For hematopoietic cells can use IFNbeta or CD45.

For Osteoblasts can use OG-2.

The promoter used to drive guide RNA can include:

Pol III promoters such as U6 or H1

Use of Pol II promoter and intronic cassettes to express gRNA

### Adeno Associated Virus (AAV)

The DNA targeting agent according to the invention as described herein, such as by means of example Cas9 and one or more guide RNA can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses may be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific genome modification, the expression of the DNA targeting agent according to the invention as described herein, such as by means of example Cas9 can be driven by a cell-type specific promoter. For example, liver-specific expression might use the Albumin promoter and neuron-specific expression (e.g. for targeting CNS disorders) might use the Synapsin I promoter.

In terms of in vivo delivery, AAV is advantageous over other viral vectors for a couple of reasons:
Low toxicity (this may be due to the purification method not requiring ultra centrifugation of cell particles that can activate the immune response)
Low probability of causing insertional mutagenesis because it doesn't integrate into the host genome.

AAV has a packaging limit of 4.5 or 4.75 Kb. This means that for instance Cas9 as well as a promoter and transcription terminator have to be all fit into the same viral vector. Constructs larger than 4.5 or 4.75 Kb will lead to significantly reduced virus production. SpCas9 is quite large, the gene itself is over 4.1 Kb, which makes it difficult for packing into AAV. Therefore embodiments of the invention include utilizing homologs of Cas9 that are shorter. For example:

| Species | Cas9 Size |
|---|---|
| Corynebacter diphtheriae | 3252 |
| Eubacterium ventriosum | 3321 |
| Streptococcus pasteurianus | 3390 |
| Lactobacillus farciminis | 3378 |
| Sphaerochaeta globus | 3537 |
| Azospirillum B510 | 3504 |
| Gluconacetobacter diazotrophicus | 3150 |
| Neisseria cinerea | 3246 |
| Roseburia intestinalis | 3420 |
| Parvibaculum lavamentivorans | 3111 |
| Staphylococcus aureus | 3159 |
| Nitratifractor salsuginis DSM 16511 | 3396 |
| Campylobacter lari CF89-12 | 3009 |
| Streptococcus thermophilus LMD-9 | 3396 |

These species are therefore, in general, preferred Cas9 species.

As to AAV, the AAV can be AAV1, AAV2, AAV5 or any combination thereof. One can select the AAV of the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The herein promoters and vectors are preferred individually. A tabulation of certain AAV serotypes as to these cells (see Grimm, D. et al, J. Virol. 82: 5887-5911 (2008)) is as follows:

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature | 2500 | 100 | ND | ND | 222 | 2857 | ND | ND |
| DC | | | | | | | | |
| Mature DC | 2222 | 100 | ND | ND | 333 | 3333 | ND | ND |

Lentivirus

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses may be prepared as follows, by means of example for Cas delivery. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media was changed to OptiMEM (serum-free) media and transfection was done 4 hours later. Cells were transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection was done in 4 mL OptiMEM with a cationic lipid delivery agent (50 uL Lipofectamine 2000 and 100 ul Plus reagent). After 6 hours, the media was changed to antibiotic-free DMEM with 10% fetal bovine serum. These methods use serum during cell culture, but serum-free methods are preferred.

Lentivirus may be purified as follows. Viral supernatants were harvested after 48 hours. Supernatants were first cleared of debris and filtered through a 0.45 um low protein binding (PVDF) filter. They were then spun in a ultracentrifuge for 2 hours at 24,000 rpm. Viral pellets were resuspended in 50 ul of DMEM overnight at 4 C. They were then aliquotted and immediately frozen at −80° C.

In another embodiment, minimal non-primate lentiviral vectors based on the equine infectious anemia virus (EIAV) are also contemplated, especially for ocular gene therapy (see, e.g., Balagaan, J Gene Med 2006; 8: 275-285). In another embodiment, RetinoStat®, an equine infectious anemia virus-based lentiviral gene therapy vector that expresses angiostatic proteins endostatin and angiostatin that is delivered via a subretinal injection for the treatment of the web form of age-related macular degeneration is also contemplated (see, e.g., Binley et al., HUMAN GENE THERAPY 23:980-991 (September 2012)) and this vector may be modified for the CRISPR-Cas system of the present invention.

In another embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the CRISPR-Cas system of the present invention. A minimum of 2.5×10⁶ CD34+ cells per kilogram patient weight may be collected and prestimulated for 16 to 20 hours in X-VIVO 15 medium (Lonza) containing 2 μmol/L-glutamine, stem cell factor (100 ng/ml), Flt-3 ligand (Flt-3L) (100 ng/ml), and thrombopoietin (10 ng/ml) (CellGenix) at a density of 2×10⁶ cells/ml. Prestimulated cells may be transduced with lentiviral at a multiplicity of infection of 5 for 16 to 24 hours in 75-cm² tissue culture flasks coated with fibronectin (25 mg/cm²) (RetroNectin, Takara Bio Inc.).

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015.

### RNA Delivery

RNA delivery: The DNA targeting agent according to the invention as described herein, such as the CRISPR enzyme, for instance a Cas9, and/or any of the present RNAs, for instance a guide RNA, can also be delivered in the form of RNA. Cas9 mRNA can be generated using in vitro transcription. For example, Cas9 mRNA can be synthesized using a PCR cassette containing the following elements: T7_promoter-kozak sequence (GCCACC)-Cas9-3′ UTR from beta globin-polyA tail (a string of 120 or more adenines). The cassette can be used for transcription by T7 polymerase. Guide RNAs can also be transcribed using in vitro transcription from a cassette containing T7_promoter-GG-guide RNA sequence.

To enhance expression and reduce possible toxicity, the CRISPR enzyme-coding sequence and/or the guide RNA can be modified to include one or more modified nucleoside e.g. using pseudo-U or 5-Methyl-C.

mRNA delivery methods are especially promising for liver delivery currently.

Much clinical work on RNA delivery has focused on RNAi or antisense, but these systems can be adapted for delivery of RNA for implementing the present invention. References below to RNAi etc. should be read accordingly.

Particle Delivery Systems and/or Formulations:

Several types of particle delivery systems and/or formulations are known to be useful in a diverse spectrum of biomedical applications. In general, a particle is defined as a small object that behaves as a whole unit with respect to its transport and properties. Particles are further classified according to diameter. Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm.

As used herein, a particle delivery system/formulation is defined as any biological delivery system/formulation which includes a particle in accordance with the present invention. A particle in accordance with the present invention is any entity having a greatest dimension (e.g. diameter) of less than 100 microns (□m). In some embodiments, inventive particles have a greatest dimension of less than 10 □m. In some embodiments, inventive particles have a greatest dimension of less than 2000 nanometers (nm). In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarisation interferometry and nuclear magnetic resonance (NMR). Characterization (dimension measurements) may be made as to native particles (i.e., preloading) or after loading of the cargo (herein cargo refers to e.g., one or more components of for instance CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. No. 8,709,843; U.S. Pat. No. 6,007,845; U.S. Pat. No. 5,855,913; U.S. Pat. No. 5,985,309; U.S. Pat. No. 5,543,158; and the publication by James E. Dahlman and Carmen Barnes et al. Nature Nanotechnology (2014) published online 11 May 2014, doi:10.1038/nnano.2014.84, concerning particles, methods of making and using them and measurements thereof.

Particles delivery systems within the scope of the present invention may be provided in any form, including but not limited to solid, semi-solid, emulsion, or colloidal particles. As such any of the delivery systems described herein, including but not limited to, e.g., lipid-based systems, liposomes, micelles, microvesicles, exosomes, or gene gun may be provided as particle delivery systems within the scope of the present invention.

### Particles

The DNA targeting agent according to the invention as described herein, such as by means of example CRISPR enzyme mRNA and guide RNA may be delivered simultaneously using particles or lipid envelopes; for instance, CRISPR enzyme and RNA of the invention, e.g., as a complex, can be delivered via a particle as in Dahlman et al., WO2015089419 A2 and documents cited therein, such as 7C1 (see, e.g., James E. Dahlman and Carmen Barnes et al. Nature Nanotechnology (2014) published online 11 May 2014, doi:10.1038/nnano.2014.84), e.g., delivery particle comprising lipid or lipidoid and hydrophilic polymer, e.g., cationic lipid and hydrophilic polymer, for instance wherein the the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5), wherein particles are formed using an efficient, multistep process wherein first, effector protein and RNA are mixed together, e.g., at a 1:1 molar ratio, e.g., at room temperature, e.g., for 30 minutes, e.g., in sterile, nuclease free 1×PBS; and separately, DOTAP, DMPC, PEG, and cholesterol as applicable for the formulation are dissolved in alcohol, e.g., 100% ethanol; and, the two solutions are mixed together to form particles containing the complexes).

For example, Su X. Fricke J, Kavanagh D G, Irvine D J (“In vitro and in vivo mRNA delivery using lipid-enveloped pH-responsive polymer nanoparticles” Mol Pharm. 2011 Jun. 6; 8(3):774-87. doi: 10.1021/mp100390w. Epub 2011 Apr. 1) describes biodegradable core-shell structured particles with a poly(β-amino ester) (PBAE) core enveloped by a phospholipid bilayer shell. These were developed for in vivo mRNA delivery. The pH-responsive PBAE component was chosen to promote endosome disruption, while the lipid surface layer was selected to minimize toxicity of the polycation core. Such are, therefore, preferred for delivering RNA of the present invention.

In one embodiment, particles based on self assembling bioadhesive polymers are contemplated, which may be applied to oral delivery of peptides, intravenous delivery of peptides and nasal delivery of peptides, all to the brain. Other embodiments, such as oral absorption and ocular delivery of hydrophobic drugs are also contemplated. The molecular envelope technology involves an engineered polymer envelope which is protected and delivered to the site of the disease (see, e.g., Mazza, M. et al. ACSNano, 2013. 7(2): 1016-1026; Siew, A., et al. Mol Pharm, 2012. 9(1):14-28; Lalatsa, A., et al. J Contr Rel, 2012. 161(2):523-36; Lalatsa, A., et al., Mol Pharm, 2012. 9(6):1665-80; Lalatsa, A., et al. Mol Pharm, 2012. 9(6):1764-74; Garrett, N. L., et al. J Biophotonics, 2012. 5(5-6):458-68; Garrett, N. L., et al. J Raman Spect, 2012. 43(5):681-688; Ahmad, S., et al. J Royal Soc Interface 2010. 7:S423-33; Uchegbu, I. F. Expert Opin Drug Deliv, 2006. 3(5):629-40; Qu, X., et al. Biomacromolecules, 2006. 7(12):3452-9 and Uchegbu. I. F., et al. Int J Pharm, 2001. 224:185-199). Doses of about 5 mg/kg are contemplated, with single or multiple doses, depending on the target tissue.

In one embodiment, particles that can deliver DNA targeting agents according to the invention as described herein, such as RNA to a cancer cell to stop tumor growth developed by Dan Anderson's lab at MIT may be used/and or adapted to the CRISPR Cas system according to certain embodiments of the present invention. In particular, the Anderson lab developed fully automated, combinatorial systems for the synthesis, purification, characterization, and formulation of new biomaterials and nanoformulations. See, e.g., Alabi et al., Proc Natl Acad Sci USA. 2013 Aug. 6; 110(32):12881-6; Zhang et al., Adv Mater. 2013 Sep. 6:25(33):4641-5; Jiang et al., Nano Lett. 2013 Mar. 13; 13(3):1059-64; Karagiannis et al., ACS Nano. 2012 Oct. 23; 6(10):8484-7; Whitehead et al., ACS Nano. 2012 Aug. 28:6(8):6922-9 and Lee et al., Nat Nanotechnol. 2012 Jun. 3:7(6):389-93.

US patent application 20110293703 relates to lipidoid compounds are also particularly useful in the administration of polynucleotides, which may be applied to deliver the DNA targeting agent according to the invention, such as for instance the CRISPR Cas system according to certain embodiments of the present invention. In one aspect, the aminoalcohol lipidoid compounds are combined with an agent to be delivered to a cell or a subject to form microparticles, particles, liposomes, or micelles. The agent to be delivered by the particles, liposomes, or micelles may be in the form of a gas, liquid, or solid, and the agent may be a polynucleotide, protein, peptide, or small molecule. The minoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

US Patent Publication No. 20110293703 also provides methods of preparing the aminoalcohol lipidoid compounds. One or more equivalents of an amine are allowed to react with one or more equivalents of an epoxide-terminated compound under suitable conditions to form an aminoalcohol lipidoid compound of the present invention. In certain embodiments, all the amino groups of the amine are fully reacted with the epoxide-terminated compound to form tertiary amines. In other embodiments, all the amino groups of the amine are not fully reacted with the epoxide-terminated compound to form tertiary amines thereby resulting in primary or secondary amines in the aminoalcohol lipidoid compound. These primary or secondary amines are left as is or may be reacted with another electrophile such as a different epoxide-terminated compound. As will be appreciated by one skilled in the art, reacting an amine with less than excess of epoxide-terminated compound will result in a plurality of different aminoalcohol lipidoid compounds with various numbers of tails. Certain amines may be fully functionalized with two epoxide-derived compound tails while other molecules will not be completely functionalized with epoxide-derived compound tails. For example, a diamine or polyamine may include one, two, three, or four epoxide-derived compound tails off the various amino moieties of the molecule resulting in primary, secondary, and tertiary amines. In certain embodiments, all the amino groups are not fully functionalized. In certain embodiments, two of the same types of epoxide-terminated compounds are used. In other embodiments, two or more different epoxide-terminated compounds are used. The synthesis of the aminoalcohol lipidoid compounds is performed with or without solvent, and the synthesis may be performed at higher temperatures ranging from 30-100 OC., preferably at approximately 50-90 OC. The prepared aminoalcohol lipidoid compounds may be optionally purified. For example, the mixture of aminoalcohol lipidoid compounds may be purified to yield an aminoalcohol lipidoid compound with a particular number of epoxide-derived compound tails. Or the mixture may be purified to yield a particular stereo- or regioisomer. The aminoalcohol lipidoid compounds may also be alkylated using an alkyl halide (e.g., methyl iodide) or other alkylating agent, and/or they may be acylated.

US Patent Publication No. 20110293703 also provides libraries of aminoalcohol lipidoid compounds prepared by the inventive methods. These aminoalcohol lipidoid compounds may be prepared and/or screened using high-throughput techniques involving liquid handlers, robots, microtiter plates, computers, etc. In certain embodiments, the aminoalcohol lipidoid compounds are screened for their ability to transfect polynucleotides or other agents (e.g., proteins, peptides, small molecules) into the cell.

US Patent Publication No. 20130302401 relates to a class of poly(beta-amino alcohols) (PBAAs) has been prepared using combinatorial polymerization. The inventive PBAAs may be used in biotechnology and biomedical applications as coatings (such as coatings of films or multilayer films for medical devices or implants), additives, materials, excipients, non-biofouling agents, micropatterning agents, and cellular encapsulation agents. When used as surface coatings, these PBAAs elicited different levels of inflammation, both in vitro and in vivo, depending on their chemical structures. The large chemical diversity of this class of materials allowed us to identify polymer coatings that inhibit macrophage activation in vitro. Furthermore, these coatings reduce the recruitment of inflammatory cells, and reduce fibrosis, following the subcutaneous implantation of carboxylated polystyrene microparticles. These polymers may be used to form polyelectrolyte complex capsules for cell encapsulation. The invention may also have many other biological applications such as antimicrobial coatings, DNA or siRNA delivery, and stem cell tissue engineering. The teachings of US Patent Publication No. 20130302401 may be applied to the DNA targeting agent according to the invention, such as for instance the CRISPR Cas system according to certain embodiments of the present invention.

In another embodiment, lipid particles (LNPs) are contemplated. An antitransthyretin small interfering RNA has been encapsulated in lipid particles and delivered to humans (see, e.g., Coelho et al., N Engl J Med 2013.369:819-29), and such a system may be adapted and applied to the CRISPR Cas system of the present invention. Doses of about 0.01 to about 1 mg per kg of body weight administered intravenously are contemplated. Medications to reduce the risk of infusion-related reactions are contemplated, such as dexamethasone, acetampinophen, diphenhydramine or cetirizine, and ranitidine are contemplated. Multiple doses of about 0.3 mg per kilogram every 4 weeks for five doses are also contemplated.

LNPs have been shown to be highly effective in delivering siRNAs to the liver (see, e.g., Tabemero et al., Cancer Discovery, April 2013, Vol. 3. No. 4, pages 363-470) and are therefore contemplated for delivering RNA encoding CRISPR Cas to the liver. A dosage of about four doses of 6 mg/kg of the LNP every two weeks may be contemplated. Tabemero et al. demonstrated that tumor regression was observed after the first 2 cycles of LNPs dosed at 0.7 mg/kg, and by the end of 6 cycles the patient had achieved a partial response with complete regression of the lymph node metastasis and substantial shrinkage of the liver tumors. A complete response was obtained after 40 doses in this patient, who has remained in remission and completed treatment after receiving doses over 26 months. Two patients with RCC and extrahepatic sites of disease including kidney, lung, and lymph nodes that were progressing following prior therapy with VEGF pathway inhibitors had stable disease at all sites for approximately 8 to 12 months, and a patient with PNET and liver metastases continued on the extension study for 18 months (36 doses) with stable disease.

However, the charge of the LNP must be taken into consideration. As cationic lipids combined with negatively charged lipids to induce nonbilayer structures that facilitate intracellular delivery. Because charged LNPs are rapidly cleared from circulation following intravenous injection, ionizable cationic lipids with pKa values below 7 were developed (see, e.g., Rosin et al, Molecular Therapy, vol. 19, no. 12, pages 1286-2200, December 2011). Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). It has been shown that LNP siRNA systems containing these lipids exhibit remarkably different gene silencing properties in hepatocytes in vivo, with potencies varying according to the series DLinKC2-DMA>DLinKDMA>DLinDMA>>DLinDAP employing a Factor VII gene silencing model (see, e.g., Rosin et al, Molecular Therapy, vol. 19, no. 12, pages 1286-2200, December 2011). A dosage of 1 μg/ml of LNP or by means of example CRISPR-Cas RNA in or associated with the LNP may be contemplated, especially for a formulation containing DLinKC2-DMA.

Preparation of LNPs and the DNA targeting agent according to the invention as described herein, such as by means of example CRISPR Cas encapsulation may be used/and or adapted from Rosin et al, Molecular Therapy, vol. 19, no. 12, pages 1286-2200, December 2011). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(o-methoxy-poly(ethylene glycol)2000) carbamoyl]-1,2-dimyristyloxlpropyl-3-amine (PEG-C-DOMG) may be provided by Tekmira Pharmaceuticals (Vancouver, Canada) or synthesized. Cholesterol may be purchased from Sigma (St Louis, Mo.). The specific CRISPR Cas RNA may be encapsulated in LNPs containing DLinDAP, DLinDMA, DLinK-DMA, and DLinKC2-DMA (cationic lipid:DSPC:CHOL:PEGS-DMG or PEG-C-DOMG at 40:10:40:10 molar ratios). When required, 0.2% SP-DiOC18 (Invitrogen. Burlington. Canada) may be incorporated to assess cellular uptake, intracellular delivery, and biodistribution. Encapsulation may be performed by dissolving lipid mixtures comprised of cationic lipid:DSPC:cholesterol:PEG-c-DOMG (40:10:40:10 molar ratio) in ethanol to a final lipid concentration of 10 mmol/l. This ethanol solution of lipid may be added drop-wise to 50 mmol/l citrate, pH 4.0 to form multilamellar vesicles to produce a final concentration of 30% ethanol vol/vol. Large unilamellar vesicles may be formed following extrusion of multilamellar vesicles through two stacked 80 nm Nuclepore polycarbonate filters using the Extruder (Northern Lipids, Vancouver, Canada). Encapsulation may be achieved by adding RNA dissolved at 2 mg/ml in 50 mmol/1 citrate, pH 4.0 containing 30% ethanol vol/vol drop-wise to extruded preformed large unilamellar vesicles and incubation at 31° C. for 30 minutes with constant mixing to a final RNA/lipid weight ratio of 0.06/1 wt/wt. Removal of ethanol and neutralization of formulation buffer were performed by dialysis against phosphate-buffered saline (PBS), pH 7.4 for 16 hours using Spectra/Por 2 regenerated cellulose dialysis membranes. Particle size distribution may be determined by dynamic light scattering using a NICOMP 370 particle sizer, the vesicle/intensity modes, and Gaussian fitting (Nicomp Particle Sizing, Santa Barbara, Calif.). The particle size for all three LNP systems may be ˜70 nm in diameter. RNA encapsulation efficiency may be determined by removal of free RNA using VivaPureD MiniH columns (Sartorius Stedim Biotech) from samples collected before and after dialysis. The encapsulated RNA may be extracted from the eluted particles and quantified at 260 nm. RNA to lipid ratio was determined by measurement of cholesterol content in vesicles using the Cholesterol E enzymatic assay from Wako Chemicals USA (Richmond, Va.). In conjunction with the herein discussion of LNPs and PEG lipids, PEGylated liposomes or LNPs are likewise suitable for delivery of a CRISPR-Cas system or components thereof.

Preparation of large LNPs may be used/and or adapted from Rosin et al, Molecular Therapy, vol. 19, no. 12, pages 1286-2200, December 2011. A lipid premix solution (20.4 mg/ml total lipid concentration) may be prepared in ethanol containing DLinKC2-DMA, DSPC, and cholesterol at 50:10:38.5 molar ratios. Sodium acetate may be added to the lipid premix at a molar ratio of 0.75:1 (sodium acetate:DLinKC2-DMA). The lipids may be subsequently hydrated by combining the mixture with 1.85 volumes of citrate buffer (10 mmol/1, pH 3.0) with vigorous stirring, resulting in spontaneous liposome formation in aqueous buffer containing 35% ethanol. The liposome solution may be incubated at 37° C. to allow for time-dependent increase in particle size. Aliquots may be removed at various times during incubation to investigate changes in liposome size by dynamic light scattering (Zetasizer Nano ZS, Malvern Instruments, Worcestershire, UK). Once the desired particle size is achieved, an aqueous PEG lipid solution (stock=10 mg/ml PEG-DMG in 35% (vol/vol) ethanol) may be added to the liposome mixture to yield a final PEG molar concentration of 3.5% of total lipid. Upon addition of PEG-lipids, the liposomes should their size, effectively quenching further growth. RNA may then be added to the empty liposomes at an RNA to total lipid ratio of approximately 1:10 (wt:wt), followed by incubation for 30 minutes at 37° C. to form loaded LNPs. The mixture may be subsequently dialyzed overnight in PBS and filtered with a 0.45-μm syringe filter.

Spherical Nucleic Acid (SNA™) constructs and other particles (particularly gold particles) are also contemplated as a means to deliver the DNA targeting agent according to the invention as described herein, such as by means of example CRISPR-Cas system to intended targets. Significant data show that AuraSense Therapeutics' Spherical Nucleic Acid (SNA™) constructs, based upon nucleic acid-functionalized gold particles, are useful.

Literature that may be employed in conjunction with herein teachings include: Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391. Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., Small, 10:186-192.

Self-assembling particles with RNA may be constructed with polyethyleneimine (PEI) that is PEGylated with an Arg-Gly-Asp (RGD) peptide ligand attached at the distal end of the polyethylene glycol (PEG). This system has been used, for example, as a means to target tumor neovasculature expressing integrins and deliver siRNA inhibiting vascular endothelial growth factor receptor-2 (VEGF R2) expression and thereby achieve tumor angiogenesis (see, e.g., Schiffelers et al., Nucleic Acids Research, 2004, Vol. 32, No. 19). Nanoplexes may be prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. A dosage of about 100 to 200 mg of CRISPR Cas is envisioned for delivery in the self-assembling particles of Schiffelers et al.

The nanoplexes of Bartlett et al. (PNAS, Sep. 25, 2007, vol. 104, no. 39) may also be applied to the present invention. The nanoplexes of Bartlett et al. are prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. The DOTA-siRNA of Bartlett et al. was synthesized as follows: 1,4,7,10-tetraazacyclododecane-1,4,7,10-tetraacetic acid mono(N-hydroxysuccinimide ester) (DOTA-NHSester) was ordered from Macrocyclics (Dallas, Tex.). The amine modified RNA sense strand with a 100-fold molar excess of DOTA-NHS-ester in carbonate buffer (pH 9) was added to a microcentrifuge tube. The contents were reacted by stirring for 4 h at room temperature. The DOTA-RNAsense conjugate was ethanol-precipitated, resuspended in water, and annealed to the unmodified antisense strand to yield DOTA-siRNA. All liquids were pretreated with Chelex-100 (Bio-Rad, Hercules, Calif.) to remove trace metal contaminants. Tf-targeted and nontargeted siRNA particles may be formed by using cyclodextrin-containing polycations. Typically, particles were formed in water at a charge ratio of 3 (+/−) and an siRNA concentration of 0.5 g/liter. One percent of the adamantane-PEG molecules on the surface of the targeted particles were modified with Tf (adamantane-PEG-Tf). The particles were suspended in a 5% (wt/vol) glucose carrier solution for injection.

Davis et al. (Nature, Vol 464, 15 Apr. 2010) conducts a RNA clinical trial that uses a targeted particle-delivery system (clinical trial registration number NCT00689065). Patients with solid cancers refractory to standard-of-care therapies are administered doses of targeted particles on days 1, 3, 8 and 10 of a 21-day cycle by a 30-min intravenous infusion. The particles consist of a synthetic delivery system containing: (1) a linear, cyclodextrin-based polymer (CDP), (2) a human transferrin protein (TF) targeting ligand displayed on the exterior of the particle to engage TF receptors (TFR) on the surface of the cancer cells, (3) a hydrophilic polymer (polyethylene glycol (PEG) used to promote particle stability in biological fluids), and (4) siRNA designed to reduce the expression of the RRM2 (sequence used in the clinic was previously denoted siR2B+5). The TFR has long been known to be upregulated in malignant cells, and RRM2 is an established anti-cancer target. These particles (clinical version denoted as CALAA-01) have been shown to be well tolerated in multi-dosing studies in non-human primates. Although a single patient with chronic myeloid leukaemia has been administered siRNA by liposomal delivery, Davis et al.'s clinical trial is the initial human trial to systemically deliver siRNA with a targeted delivery system and to treat patients with solid cancer. To ascertain whether the targeted delivery system can provide effective delivery of functional siRNA to human tumours, Davis et al. investigated biopsies from three patients from three different dosing cohorts; patients A, B and C, all of whom had metastatic melanoma and received CALAA-01 doses of 18, 24 and 30 mg m⁻² siRNA, respectively. Similar doses may also be contemplated for the CRISPR Cas system of the present invention. The delivery of the invention may be achieved with particles containing a linear, cyclodextrin-based polymer (CDP), a human transferrin protein (TF) targeting ligand displayed on the exterior of the particle to engage TF receptors (TFR) on the surface of the cancer cells and/or a hydrophilic polymer (for example, polyethylene glycol (PEG) used to promote particle stability in biological fluids).

In terms of this invention, it is preferred to have one or more components of the DNA targeting agent according to the invention as described herein, such as by means of example the CRISPR complex, e.g., CRISPR enzyme or mRNA or guide RNA delivered using particles or lipid envelopes. Other delivery systems or vectors are may be used in conjunction with the particle aspects of the invention.

In general, a “nanoparticle” refers to any particle having a diameter of less than 100) nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, particles of the invention have a greatest dimension ranging between 35 nm and 60 nm. In other preferred embodiments, the particles of the invention are not nanoparticles.

Particles encompassed in the present invention may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles). Particles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention.

Semi-solid and soft particles have been manufactured, and are within the scope of the present invention. A prototype particle of semi-solid nature is the liposome. Various types of liposome particles are currently used clinically as delivery systems for anticancer drugs and vaccines. Particles with one half hydrophilic and the other half hydrophobic are termed Janus particles and are particularly effective for stabilizing emulsions. They can self-assemble at water/oil interfaces and act as solid surfactants.

U.S. Pat. No. 8,709,843, incorporated herein by reference, provides a drug delivery system for targeted delivery of therapeutic agent-containing particles to tissues, cells, and intracellular compartments. The invention provides targeted particles comprising comprising polymer conjugated to a surfactant, hydrophilic polymer or lipid. U.S. Pat. No. 6,007,845, incorporated herein by reference, provides particles which have a core of a multiblock copolymer formed by covalently linking a multifunctional compound with one or more hydrophobic polymers and one or more hydrophilic polymers, and contain a biologically active material. U.S. Pat. No. 5,855,913, incorporated herein by reference, provides a particulate composition having aerodynamically light particles having a tap density of less than 0.4 g/cm3 with a mean diameter of between 5 μm and 30 μm, incorporating a surfactant on the surface thereof for drug delivery to the pulmonary system. U.S. Pat. No. 5,985,309, incorporated herein by reference, provides particles incorporating a surfactant and/or a hydrophilic or hydrophobic complex of a positively or negatively charged therapeutic or diagnostic agent and a charged molecule of opposite charge for delivery to the pulmonary system. U.S. Pat. No. 5,543,158, incorporated herein by reference, provides biodegradable injectable particles having a biodegradable solid core containing a biologically active material and poly(alkylene glycol) moieties on the surface. WO2012135025 (also published as US20120251560), incorporated herein by reference, describes conjugated polyethyleneimine (PEI) polymers and conjugated aza-macrocycles (collectively referred to as “conjugated lipomer” or “lipomers”). In certain embodiments, it can envisioned that such conjugated lipomers can be used in the context of the CRISPR-Cas system to achieve in vitro, ex vivo and in vivo genomic perturbations to modify gene expression, including modulation of protein expression.

In one embodiment, the particle may be epoxide-modified lipid-polymer, advantageously 7C1 (see, e.g., James E. Dahlman and Carmen Barnes et al. Nature Nanotechnology (2014) published online 11 May 2014, doi:10.1038/nnano.2014.84). C71 was synthesized by reacting C15 epoxide-terminated lipids with PEI600 at a 14:1 molar ratio, and was formulated with C14PEG2000 to produce particles (diameter between 35 and 60 nm) that were stable in PBS solution for at least 40 days.

An epoxide-modified lipid-polymer may be utilized to deliver the CRISPR-Cas system of the present invention to pulmonary, cardiovascular or renal cells, however, one of skill in the art may adapt the system to deliver to other target organs. Dosage ranging from about 0.05 to about 0.6 mg/kg are envisioned. Dosages over several days or weeks are also envisioned, with a total dosage of about 2 mg/kg.

### Exosomes

Exosomes are endogenous nano-vesicles that transport RNAs and proteins, and which can deliver RNA to the brain and other target organs. To reduce immunogenicity, Alvarez-Erviti et al. (2011, Nat Biotechnol 29: 341) used self-derived dendritic cells for exosome production. Targeting to the brain was achieved by engineering the dendritic cells to express Lamp2b, an exosomal membrane protein, fused to the neuron-specific RVG peptide. Purified exosomes were loaded with exogenous RNA by electroporation. Intravenously injected RVG-targeted exosomes delivered GAPDH siRNA specifically to neurons, microglia, oligodendrocytes in the brain, resulting in a specific gene knockdown. Pre-exposure to RVG exosomes did not attenuate knockdown, and non-specific uptake in other tissues was not observed. The therapeutic potential of exosome-mediated siRNA delivery was demonstrated by the strong mRNA (60%) and protein (62%) knockdown of BACE1, a therapeutic target in Alzheimer's disease.

To obtain a pool of immunologically inert exosomes, Alvarez-Erviti et al. harvested bone marrow from inbred C57BL/6 mice with a homogenous major histocompatibility complex (MHC) haplotype. As immature dendritic cells produce large quantities of exosomes devoid of T-cell activators such as MHC-II and CD86. Alvarez-Erviti et al. selected for dendritic cells with granulocyte/macrophage-colony stimulating factor (GM-CSF) for 7 d. Exosomes were purified from the culture supernatant the following day using well-established ultracentrifugation protocols. The exosomes produced were physically homogenous, with a size distribution peaking at 80 nm in diameter as determined by particle tracking analysis (NTA) and electron microscopy. Alvarez-Erviti et al. obtained 6-12 μg of exosomes (measured based on protein concentration) per 10⁶ cells.

Next, Alvarez-Erviti et al. investigated the possibility of loading modified exosomes with exogenous cargoes using electroporation protocols adapted for nanoscale applications. As electroporation for membrane particles at the nanometer scale is not well-characterized, nonspecific Cy5-labeled RNA was used for the empirical optimization of the electroporation protocol. The amount of encapsulated RNA was assayed after ultracentrifugation and lysis of exosomes. Electroporation at 400 V and 125 μF resulted in the greatest retention of RNA and was used for all subsequent experiments.

Alvarez-Erviti et al. administered 150 μg of each BACE1 siRNA encapsulated in 150 μg of RVG exosomes to normal C57BL/6 mice and compared the knockdown efficiency to four controls: untreated mice, mice injected with RVG exosomes only, mice injected with BACE1 siRNA complexed to an in vivo cationic liposome reagent and mice injected with BACE1 siRNA complexed to RVG-9R, the RVG peptide conjugated to 9 D-arginines that electrostatically binds to the siRNA. Cortical tissue samples were analyzed 3 d after administration and a significant protein knockdown (45%, P<0.05, versus 62%, P<0.01) in both siRNA-RVG-9R-treated and siRNARVG exosome-treated mice was observed, resulting from a significant decrease in BACE1 mRNA levels (66% [+ or −] 15%, P<0.001 and 61% [+ or −] 13% respectively, P<0.01). Moreover, Applicants demonstrated a significant decrease (55%, P<0.05) in the total [beta]-amyloid 1-42 levels, a main component of the amyloid plaques in Alzheimer's pathology, in the RVG-exosome-treated animals. The decrease observed was greater than the β-amyloid 1-40 decrease demonstrated in normal mice after intraventricular injection of BACE1 inhibitors. Alvarez-Erviti et al. carried out 5′-rapid amplification of cDNA ends (RACE) on BACE1 cleavage product, which provided evidence of RNAi-mediated knockdown by the siRNA.

Finally, Alvarez-Erviti et al. investigated whether RNA-RVG exosomes induced immune responses in vivo by assessing IL-6, IP-10, TNFα and IFN-α serum concentrations. Following exosome treatment, nonsignificant changes in all cytokines were registered similar to siRNA-transfection reagent treatment in contrast to siRNA-RVG-9R, which potently stimulated IL-6 secretion, confirming the immunologically inert profile of the exosome treatment. Given that exosomes encapsulate only 20% of siRNA, delivery with RVG-exosome appears to be more efficient than RVG-9R delivery as comparable mRNA knockdown and greater protein knockdown was achieved with fivefold less siRNA without the corresponding level of immune stimulation. This experiment demonstrated the therapeutic potential of RVG-exosome technology, which is potentially suited for long-term silencing of genes related to neurodegenerative diseases. The exosome delivery system of Alvarez-Erviti et al. may be applied to deliver the DNA targeting agent according to the invention as described herein, such as by means of example the CRISPR-Cas system of the present invention to therapeutic targets, especially neurodegenerative diseases. A dosage of about 100 to 1000 mg of CRISPR Cas encapsulated in about 100 to 1000 mg of RVG exosomes may be contemplated for the present invention.

El-Andaloussi et al. (Nature Protocols 7,2112-2126(2012)) discloses how exosomes derived from cultured cells can be harnessed for delivery of RNA in vitro and in vivo. This protocol first describes the generation of targeted exosomes through transfection of an expression vector, comprising an exosomal protein fused with a peptide ligand. Next, El-Andaloussi et al. explain how to purify and characterize exosomes from transfected cell supernatant. Next, El-Andaloussi et al. detail crucial steps for loading RNA into exosomes. Finally, El-Andaloussi et al. outline how to use exosomes to efficiently deliver RNA in vitro and in vivo in mouse brain. Examples of anticipated results in which exosome-mediated RNA delivery is evaluated by functional assays and imaging are also provided. The entire protocol takes ˜3 weeks. Delivery or administration according to the invention may be performed using exosomes produced from self-derived dendritic cells. From the herein teachings, this can be employed in the practice of the invention.

In another embodiment, the plasma exosomes of Wahlgren et al. (Nucleic Acids Research, 2012. Vol. 40, No. 17 e130) are contemplated. Exosomes are nano-sized vesicles (30-90 nm in size) produced by many cell types, including dendritic cells (DC), B cells. T cells, mast cells, epithelial cells and tumor cells. These vesicles are formed by inward budding of late endosomes and are then released to the extracellular environment upon fusion with the plasma membrane. Because exosomes naturally carry RNA between cells, this property may be useful in gene therapy, and from this disclosure can be employed in the practice of the instant invention.

Exosomes from plasma can be prepared by centrifugation of buffy coat at 900 g for 20 min to isolate the plasma followed by harvesting cell supernatants, centrifuging at 300 g for 10 min to eliminate cells and at 16 500 g for 30 min followed by filtration through a 0.22 mm filter. Exosomes are pelleted by ultracentrifugation at 120 000 g for 70 min. Chemical transfection of siRNA into exosomes is carried out according to the manufacturer's instructions in RNAi Human/Mouse Starter Kit (Quiagen, Hilden, Germany), siRNA is added to 100 ml PBS at a final concentration of 2 mmol/ml. After adding HiPerFect transfection reagent, the mixture is incubated for 10 min at RT. In order to remove the excess of micelles, the exosomes are re-isolated using aldehyde/sulfate latex beads. The chemical transfection of CRISPR Cas into exosomes may be conducted similarly to siRNA. The exosomes may be co-cultured with monocytes and lymphocytes isolated from the peripheral blood of healthy donors. Therefore, it may be contemplated that exosomes containing the DNA targeting agent according to the invention as described herein, such as by means of example CRISPR Cas may be introduced to monocytes and lymphocytes of and autologously reintroduced into a human. Accordingly, delivery or administration according to the invention may be performed using plasma exosomes.

### Liposomes

Delivery or administration according to the invention can be performed with liposomes. Liposomes are spherical vesicle structures composed of a uni- or multilamellar lipid bilayer surrounding internal aqueous compartments and a relatively impermeable outer lipophilic phospholipid bilayer. Liposomes have gained considerable attention as drug delivery carriers because they are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi: 10.1155/2011/469679 for review).

Liposomes can be made from several different types of lipids; however, phospholipids are most commonly used to generate liposomes as drug carriers. Although liposome formation is spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi: 10.1155/2011/469679 for review).

Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. Further, liposomes are prepared from hydrogenated egg phosphatidylcholine or egg phosphatidylcholine, cholesterol, and dicetyl phosphate, and their mean vesicle sizes were adjusted to about 50 and 100 nm. (see, e.g., Spuch and Navarro, Journal of Drug Delivery, vol. 2011, Article ID 469679, 12 pages, 2011. doi: 10.1155/2011/469679 for review).

A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside. Since this formulation is made up of phospholipids only, liposomal formulations have encountered many challenges, one of the ones being the instability in plasma. Several attempts to overcome these challenges have been made, specifically in the manipulation of the lipid membrane. One of these attempts focused on the manipulation of cholesterol. Addition of cholesterol to conventional formulations reduces rapid release of the encapsulated bioactive compound into the plasma or 1,2-dioleoyl-sn-glycero-3-phosphoethanolamine (DOPE) increases the stability (see, e.g., Spuch and Navarro, Journal of Drug Delivery, vol. 2011, Article ID 469679, 12 pages, 2011. doi: 10.1155/2011/469679 for review).

In a particularly advantageous embodiment, Trojan Horse liposomes (also known as Molecular Trojan Horses) are desirable and protocols may be found at cshprotocols.cshlp.org/content/2010/4/pdb.prot5407.long. These particles allow delivery of a transgene to the entire brain after an intravascular injection. Without being bound by limitation, it is believed that neutral lipid particles with specific antibodies conjugated to surface allow crossing of the blood brain barrier via endocytosis. Applicant postulates utilizing Trojan Horse Liposomes to deliver the DNA targeting agent according to the invention as described herein, such as by means of example the CRISPR family of nucleases to the brain via an intravascular injection, which would allow whole brain transgenic animals without the need for embryonic manipulation. About 1-5 g of DNA or RNA may be contemplated for in vivo administration in liposomes.

In another embodiment, the DNA targeting agent according to the invention as described herein, such as by means of example the CRISPR Cas system may be administered in liposomes, such as a stable nucleic-acid-lipid particle (SNALP) (see, e.g., Morrissey et al., Nature Biotechnology, Vol. 23, No. 8, August 2005). Daily intravenous injections of about 1, 3 or 5 mg/kg/day of a specific CRISPR Cas targeted in a SNALP are contemplated. The daily treatment may be over about three days and then weekly for about five weeks. In another embodiment, a specific CRISPR Cas encapsulated SNALP) administered by intravenous injection to at doses of about 1 or 2.5 mg/kg are also contemplated (see, e.g., Zimmerman et al., Nature Letters, Vol. 441, 4 May 2006). The SNALP formulation may contain the lipids 3-N-[(methoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio (see, e.g., Zimmerman et al., Nature Letters, Vol. 441, 4 May 2006).

In another embodiment, stable nucleic-acid-lipid particles (SNALPs) have proven to be effective delivery molecules to highly vascularized HepG2-derived liver tumors but not in poorly vascularized HCT-116 derived liver tumors (see, e.g., Li, Gene Therapy (2012) 19, 775-780). The SNALP liposomes may be prepared by formulating D-Lin-DMA and PEG-C-DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulted SNALP liposomes are about 80-100 nm in size.

In yet another embodiment, a SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)2000)carbamoyl]-1,2-dimyrestyloxypropylamine, and cationic 1,2-dilinoleyloxy-3-N,Ndimethylaminopropane (see, e.g., Geisbert et al., Lancet 2010; 375: 1896-905). A dosage of about 2 mg/kg total CRISPR Cas per dose administered as, for example, a bolus intravenous infusion may be contemplated.

In yet another embodiment, a SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N;N-dimethyl)aminopropane (DLinDMA) (see, e.g., Judge. J. Clin. Invest. 119:661-673 (2009)). Formulations used for in vivo studies may comprise a final lipid/RNA mass ratio of about 9:1.

The safety profile of RNAi nanomedicines has been reviewed by Barros and Gollob of Alnylam Pharmaceuticals (see, e.g., Advanced Drug Delivery Reviews 64 (2012) 1730-1737). The stable nucleic acid lipid particle (SNALP) is comprised of four different lipids—an ionizable lipid (DLinDMA) that is cationic at low pH, a neutral helper lipid, cholesterol, and a diffusible polyethylene glycol (PEG)-lipid. The particle is approximately 80 nm in diameter and is charge-neutral at physiologic pH. During formulation, the ionizable lipid serves to condense lipid with the anionic RNA during particle formation. When positively charged under increasingly acidic endosomal conditions, the ionizable lipid also mediates the fusion of SNALP with the endosomal membrane enabling release of RNA into the cytoplasm. The PEG-lipid stabilizes the particle and reduces aggregation during formulation, and subsequently provides a neutral hydrophilic exterior that improves pharmacokinetic properties.

To date, two clinical programs have been initiated using SNALP formulations with RNA. Tekmira Pharmaceuticals recently completed a phase I single-dose study of SNALP-ApoB in adult volunteers with elevated LDL cholesterol. ApoB is predominantly expressed in the liver and jejunum and is essential for the assembly and secretion of VLDL and LDL. Seventeen subjects received a single dose of SNALP-ApoB (dose escalation across 7 dose levels). There was no evidence of liver toxicity (anticipated as the potential dose-limiting toxicity based on preclinical studies). One (of two) subjects at the highest dose experienced flu-like symptoms consistent with immune system stimulation, and the decision was made to conclude the trial.

Alnylam Pharmaceuticals has similarly advanced ALN-TTR01, which employs the SNALP technology described above and targets hepatocyte production of both mutant and wild-type TTR to treat TTR amyloidosis (ATTR). Three ATTR syndromes have been described: familial amyloidotic polyneuropathy (FAP) and familial amyloidotic cardiomyopathy (FAC)—both caused by autosomal dominant mutations in TTR; and senile systemic amyloidosis (SSA) cause by wildtype TTR. A placebo-controlled, single dose-escalation phase I trial of ALN-TTR01 was recently completed in patients with ATTR. ALN-TTR01 was administered as a 15-minute IV infusion to 31 patients (23 with study drug and 8 with placebo) within a dose range of 0.01 to 1.0 mg/kg (based on siRNA). Treatment was well tolerated with no significant increases in liver function tests. Infusion-related reactions were noted in 3 of 23 patients at >0.4 mg/kg; all responded to slowing of the infusion rate and all continued on study. Minimal and transient elevations of serum cytokines IL-6, IP-10 and IL-1ra were noted in two patients at the highest dose of 1 mg/kg (as anticipated from preclinical and NHP studies). Lowering of serum TTR, the expected pharmacodynamics effect of ALN-TTR01, was observed at 1 mg/kg.

In yet another embodiment, a SNALP may be made by solubilizing a cationic lipid. DSPC, cholesterol and PEG-lipid e.g., in ethanol, e.g., at a molar ratio of 40:10:40:10, respectively (see, Semple et al., Nature Niotechnology. Volume 28 Number 2 Feb. 2010, pp. 172-177). The lipid mixture was added to an aqueous buffer (50 mM citrate, pH 4) with mixing to a final ethanol and lipid concentration of 30% (vol/vol) and 6.1 mg/ml, respectively, and allowed to equilibrate at 22° C. for 2 min before extrusion. The hydrated lipids were extruded through two stacked 80 nm pore-sized filters (Nuclepore) at 22° C. using a Lipex Extruder (Northern Lipids) until a vesicle diameter of 70-90 nm, as determined by dynamic light scattering analysis, was obtained. This generally required 1-3 passes. The siRNA (solubilized in a 50 mM citrate, pH 4 aqueous solution containing 30% ethanol) was added to the pre-equilibrated (35° C.) vesicles at a rate of ˜5 ml/min with mixing. After a final target siRNA/lipid ratio of 0.06 (wt/wt) was reached, the mixture was incubated for a further 30 min at 35° C. to allow vesicle reorganization and encapsulation of the siRNA. The ethanol was then removed and the external buffer replaced with PBS (155 mM NaCl, 3 mM Na₂HPO₄, 1 mM KH₂PO₄, pH 7.5) by either dialysis or tangential flow diafiltration. siRNA were encapsulated in SNALP using a controlled step-wise dilution method process. The lipid constituents of KC2-SNALP were DLin-KC2-DMA (cationic lipid), dipalmitoylphosphatidylcholine (DPPC; Avanti Polar Lipids), synthetic cholesterol (Sigma) and PEG-C-DMA used at a molar ratio of 57.1:7.1:34.3:1.4. Upon formation of the loaded particles, SNALP were dialyzed against PBS and filter sterilized through a 0.2 μm filter before use. Mean particle sizes were 75-85 nm and 90-95% of the siRNA was encapsulated within the lipid particles. The final siRNA/lipid ratio in formulations used for in vivo testing was ˜0.15 (wt/wt). LNP-siRNA systems containing Factor VII siRNA were diluted to the appropriate concentrations in sterile PBS immediately before use and the formulations were administered intravenously through the lateral tail vein in a total volume of 10 ml/kg. This method and these delivery systems may be extrapolated to the CRISPR Cas system of the present invention.

### Other Lipids

Other cationic lipids, such as amino lipid 2,2-dilinoleyl-4-dimethylaminoethyl-[1,3]-dioxolane (DLin-KC2-DMA) may be utilized to encapsulate the DNA targeting agent according to the invention as described herein, such as by means of example CRISPR Cas or components thereof or nucleic acid molecule(s) coding therefor e.g., similar to SiRNA (see, e.g., Jayaraman, Angew. Chem. Int. Ed. 2012, 51, 8529-8533), and hence may be employed in the practice of the invention. A preformed vesicle with the following lipid composition may be contemplated: amino lipid, distearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11±0.04 (n=56), the particles may be extruded up to three times through 80 nm membranes prior to adding the CRISPR Cas RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Michael S D Kormann et al. (“Expression of therapeutic proteins after delivery of chemically modified mRNA in mice: Nature Biotechnology, Volume: 29, Pages: 154-157 (2011)) describes the use of lipid envelopes to deliver RNA. Use of lipid envelopes is also preferred in the present invention.

In another embodiment, lipids may be formulated with the CRISPR Cas system of the present invention to form lipid particles (LNPs). Lipids include, but are not limited to, DLin-KC2-DMA4, C12-200 and colipids disteroylphosphatidyl choline, cholesterol, and PEG-DMG may be formulated with CRISPR Cas instead of siRNA (see, e.g., Novobrantseva. Molecular Therapy-Nucleic Acids (2012) 1, e4; doi:10.1038/mtna.2011.3) using a spontaneous vesicle formation procedure. The component molar ratio may be about 50/10/38.5/1.5 (DLin-KC2-DMA or C12-200/disteroylphosphatidyl choline/cholesterol/PEG-DMG). The final lipid:siRNA weight ratio may be ˜12:1 and 9:1 in the case of DLin-KC2-DMA and C12-200 lipid particles (LNPs), respectively. The formulations may have mean particle diameters of ˜80 nm with >90% entrapment efficiency. A 3 mg/kg dose may be contemplated.

Tekmira has a portfolio of approximately 95 patent families, in the U.S. and abroad, that are directed to various aspects of LNPs and LNP formulations (see. e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316), all of which may be used and/or adapted to the present invention.

The DNA targeting agent according to the invention as described herein, such as by means of example CRISPR Cas system or components thereof or nucleic acid molecule(s) coding therefor may be delivered encapsulated in PLGA Microspheres such as that further described in US published applications 20130252281 and 20130245107 and 20130244279 (assigned to Moderna Therapeutics) which relate to aspects of formulation of compositions comprising modified nucleic acid molecules which may encode a protein, a protein precursor, or a partially or fully processed form of the protein or a protein precursor. The formulation may have a molar ratio 50:10:38.5:1.5-3.0 (cationic lipid:fusogenic lipid:cholesterol:PEG lipid). The PEG lipid may be selected from, but is not limited to PEG-c-DOMG. PEG-DMG. The fusogenic lipid may be DSPC. See also, Schrum et al., Delivery and Formulation of Engineered Nucleic Acids, US published application 20120251618.

Nanomerics' technology addresses bioavailability challenges for a broad range of therapeutics, including low molecular weight hydrophobic drugs, peptides, and nucleic acid based therapeutics (plasmid, siRNA, miRNA). Specific administration routes for which the technology has demonstrated clear advantages include the oral route, transport across the blood-brain-barrier, delivery to solid tumours, as well as to the eye. See, e.g., Mazza et al., 2013, ACS Nano. 2013 Feb. 26; 7(2):1016-26; Uchegbu and Siew, 2013, J Pharm Sci. 102(2):305-10 and Lalatsa et al., 2012, J Control Release. 2012 Jul. 20; 161(2):523-36.

US Patent Publication No. 20050019923 describes cationic dendrimers for delivering bioactive molecules, such as polynucleotide molecules, peptides and polypeptides and/or pharmaceutical agents, to a mammalian body. The dendrimers are suitable for targeting the delivery of the bioactive molecules to, for example, the liver, spleen, lung, kidney or heart (or even the brain). Dendrimers are synthetic 3-dimensional macromolecules that are prepared in a step-wise fashion from simple branched monomer units, the nature and functionality of which can be easily controlled and varied. Dendrimers are synthesised from the repeated addition of building blocks to a multifunctional core (divergent approach to synthesis), or towards a multifunctional core (convergent approach to synthesis) and each addition of a 3-dimensional shell of building blocks leads to the formation of a higher generation of the dendrimers. Polypropylenimine dendrimers start from a diaminobutane core to which is added twice the number of amino groups by a double Michael addition of acrylonitrile to the primary amines followed by the hydrogenation of the nitriles. This results in a doubling of the amino groups. Polypropylenimine dendrimers contain 100% protonable nitrogens and up to 64 terminal amino groups (generation 5, DAB 64). Protonable groups are usually amine groups which are able to accept protons at neutral pH. The use of dendrimers as gene delivery agents has largely focused on the use of the polyamidoamine and phosphorous containing compounds with a mixture of amine/amide or N—P(O₂)S as the conjugating units respectively with no work being reported on the use of the lower generation polypropylenimine dendrimers for gene delivery. Polypropylenimine dendrimers have also been studied as pH sensitive controlled release systems for drug delivery and for their encapsulation of guest molecules when chemically modified by peripheral amino acid groups. The cytotoxicity and interaction of polypropylenimine dendrimers with DNA as well as the transfection efficacy of DAB 64 has also been studied.

US Patent Publication No. 20050019923 is based upon the observation that, contrary to earlier reports, cationic dendrimers, such as polypropylenimine dendrimers, display suitable properties, such as specific targeting and low toxicity, for use in the targeted delivery of bioactive molecules, such as genetic material. In addition, derivatives of the cationic dendrimer also display suitable properties for the targeted delivery of bioactive molecules. See also, Bioactive Polymers, US published application 20080267903, which discloses “Various polymers, including cationic polyamine polymers and dendrimeric polymers, are shown to possess anti-proliferative activity, and may therefore be useful for treatment of disorders characterised by undesirable cellular proliferation such as neoplasms and tumours, inflammatory disorders (including autoimmune disorders), psoriasis and atherosclerosis. The polymers may be used alone as active agents, or as delivery vehicles for other therapeutic agents, such as drug molecules or nucleic acids for gene therapy. In such cases, the polymers' own intrinsic anti-tumour activity may complement the activity of the agent to be delivered.” The disclosures of these patent publications may be employed in conjunction with herein teachings for delivery of CRISPR Cas system(s) or component(s) thereof or nucleic acid molecule(s) coding therefor.

### Supercharged Proteins

Supercharged proteins are a class of engineered or naturally occurring proteins with unusually high positive or negative net theoretical charge and may be employed in delivery of the DNA targeting agent according to the invention as described herein, such as by means of example CRISPR Cas system(s) or component(s) thereof or nucleic acid molecule(s) coding therefor. Both supernegatively and superpositively charged proteins exhibit a remarkable ability to withstand thermally or chemically induced aggregation. Superpositively charged proteins are also able to penetrate mammalian cells. Associating cargo with these proteins, such as plasmid DNA, RNA, or other proteins, can enable the functional delivery of these macromolecules into mammalian cells both in vitro and in vivo. David Liu's lab reported the creation and characterization of supercharged proteins in 2007 (Lawrence et al., 2007, Journal of the American Chemical Society 129, 10110-10112).

The nonviral delivery of RNA and plasmid DNA into mammalian cells are valuable both for research and therapeutic applications (Akinc et al., 2010, Nat. Biotech. 26, 561-569). Purified +36 GFP protein (or other superpositively charged protein) is mixed with RNAs in the appropriate serum-free media and allowed to complex prior addition to cells. Inclusion of serum at this stage inhibits formation of the supercharged protein-RNA complexes and reduces the effectiveness of the treatment. The following protocol has been found to be effective for a variety of cell lines (McNaughton et al., 2009. Proc. Natl. Acad. Sci. USA 106, 6111-6116) (However, pilot experiments varying the dose of protein and RNA should be performed to optimize the procedure for specific cell lines): (1) One day before treatment, plate 1×10⁵ cells per well in a 48-well plate. (2) On the day of treatment, dilute purified +36 GFP protein in serumfree media to a final concentration 200 nM. Add RNA to a final concentration of 50 nM. Vortex to mix and incubate at room temperature for 10 min. (3) During incubation, aspirate media from cells and wash once with PBS. (4) Following incubation of +36 GFP and RNA, add the protein-RNA complexes to cells. (5) Incubate cells with complexes at 37° C. for 4 h. (6) Following incubation, aspirate the media and wash three times with 20 U/mL heparin PBS. Incubate cells with serum-containing media for a further 48 h or longer depending upon the assay for activity. (7) Analyze cells by immunoblot, qPCR, phenotypic assay, or other appropriate method.

David Liu's lab has further found +36 GFP to be an effective plasmid delivery reagent in a range of cells. As plasmid DNA is a larger cargo than siRNA, proportionately more +36 GFP protein is required to effectively complex plasmids. For effective plasmid delivery Applicants have developed a variant of +36 GFP bearing a C-terminal HA2 peptide tag, a known endosome-disrupting peptide derived from the influenza virus hemagglutinin protein. The following protocol has been effective in a variety of cells, but as above it is advised that plasmid DNA and supercharged protein doses be optimized for specific cell lines and delivery applications: (1) One day before treatment, plate 1×10⁵ per well in a 48-well plate. (2) On the day of treatment, dilute purified b36 GFP protein in serumfree media to a final concentration 2 mM. Add 1 mg of plasmid DNA. Vortex to mix and incubate at room temperature for 10 min. (3) During incubation, aspirate media from cells and wash once with PBS. (4) Following incubation of b36 GFP and plasmid DNA, gently add the protein-DNA complexes to cells. (5) Incubate cells with complexes at 37 C for 4 h. (6) Following incubation, aspirate the media and wash with PBS. Incubate cells in serum-containing media and incubate for a further 24-48 h. (7) Analyze plasmid delivery (e.g., by plasmid-driven gene expression) as appropriate. See also, e.g., McNaughton et al., Proc. Natl. Acad. Sci. USA 106, 6111-6116 (2009); Cronican et al., ACS Chemical Biology 5, 747-752 (2010); Cronican et al., Chemistry & Biology 18, 833-838 (2011): Thompson et al., Methods in Enzymology 503, 293-319 (2012); Thompson, D. B., et al., Chemistry & Biology 19 (7), 831-843 (2012). The methods of the super charged proteins may be used and/or adapted for delivery of the CRISPR Cas system of the present invention. These systems of Dr. Lui and documents herein in inconjunction with herein teachings can be employed in the delivery of the DNA targeting agent according to the invention as described herein, such as by means of example CRISPR Cas system(s) or component(s) thereof or nucleic acid molecule(s) coding therefor.

### Cell Penetrating Peptides (CPPs)

In yet another embodiment, cell penetrating peptides (CPPs) are contemplated for the delivery of the DNA targeting agent according to the invention as described herein, such as by means of example CRISPR Cas system. CPPs are short peptides that facilitate cellular uptake of various molecular cargo (from nanosize particles to small chemical molecules and large fragments of DNA). The term “cargo” as used herein includes but is not limited to the group consisting of therapeutic agents, diagnostic probes, peptides, nucleic acids, antisense oligonucleotides, plasmids, proteins, particles, liposomes, chromophores, small molecules and radioactive materials. In aspects of the invention, the cargo may also comprise any component of the DNA targeting agent according to the invention as described herein, such as by means of example CRISPR Cas system or the entire functional CRISPR Cas system. Aspects of the present invention further provide methods for delivering a desired cargo into a subject comprising: (a) preparing a complex comprising the cell penetrating peptide of the present invention and a desired cargo, and (b) orally, intraarticularly, intraperitoneally, intrathecally, intraarterially, intranasally, intraparenchymally, subcutaneously, intramuscularly, intravenously, dermally, intrarectally, or topically administering the complex to a subject. The cargo is associated with the peptides either through chemical linkage via covalent bonds or through non-covalent interactions.

The function of the CPPs are to deliver the cargo into cells, a process that commonly occurs through endocytosis with the cargo delivered to the endosomes of living mammalian cells. Cell-penetrating peptides are of different sizes, amino acid sequences, and charges but all CPPs have one distinct characteristic, which is the ability to translocate the plasma membrane and facilitate the delivery of various molecular cargoes to the cytoplasm or an organelle. CPP translocation may be classified into three main entry mechanisms: direct penetration in the membrane, endocytosis-mediated entry, and translocation through the formation of a transitory structure. CPPs have found numerous applications in medicine as drug delivery agents in the treatment of different diseases including cancer and virus inhibitors, as well as contrast agents for cell labeling. Examples of the latter include acting as a carrier for GFP, MRI contrast agents, or quantum dots. CPPs hold great potential as in vitro and in vivo delivery vectors for use in research and medicine. CPPs typically have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. One of the initial CPPs discovered was the trans-activating transcriptional activator (Tat) from Human Immunodeficiency Virus 1 (HIV-1) which was found to be efficiently taken up from the surrounding media by numerous cell types in culture. Since then, the number of known CPPs has expanded considerably and small molecule synthetic analogues with more effective protein transduction properties have been generated. CPPs include but are not limited to Penetratin, Tat (48-60), Transportan, and (R-AhX-R)4 (SEQ ID NO: 17) (Ahx=aminohexanoyl).

U.S. Pat. No. 8,372,951, provides a CPP derived from eosinophil cationic protein (ECP) which exhibits highly cell-penetrating efficiency and low toxicity. Aspects of delivering the CPP with its cargo into a vertebrate subject are also provided. Further aspects of CPPs and their delivery are described in U.S. Pat. Nos. 8,575,305; 8,614,194 and 8,044,019. CPPs can be used to deliver the CRISPR-Cas system or components thereof. That CPPs can be employed to deliver the CRISPR-Cas system or components thereof is also provided in the manuscript “Gene disruption by cell-penetrating peptide-mediated delivery of Cas9 protein and guide RNA”, by Suresh Ramakrishna, Abu-Bonsrah Kwaku Dad, Jagadish Beloor, et al. Genome Res. 2014 Apr. 2. [Epub ahead of print], incorporated by reference in its entirety, wherein it is demonstrated that treatment with CPP-conjugated recombinant Cas9 protein and CPP-complexed guide RNAs lead to endogenous gene disruptions in human cell lines. In the paper the Cas9 protein was conjugated to CPP via a thioether bond, whereas the guide RNA was complexed with CPP, forming condensed, positively charged particles. It was shown that simultaneous and sequential treatment of human cells, including embryonic stem cells, dermal fibroblasts, HEK293T cells, HeLa cells, and embryonic carcinoma cells, with the modified Cas9 and guide RNA led to efficient gene disruptions with reduced off-target mutations relative to plasmid transfections.

### Implantable Devices

In another embodiment, implantable devices are also contemplated for delivery of the DNA targeting agent according to the invention as described herein, such as by means of example the CRISPR Cas system or component(s) thereof or nucleic acid molecule(s) coding therefor. For example, US Patent Publication 20110195123 discloses an implantable medical device which elutes a drug locally and in prolonged period is provided, including several types of such a device, the treatment modes of implementation and methods of implantation. The device comprising of polymeric substrate, such as a matrix for example, that is used as the device body, and drugs, and in some cases additional scaffolding materials, such as metals or additional polymers, and materials to enhance visibility and imaging. An implantable delivery device can be advantageous in providing release locally and over a prolonged period, where drug is released directly to the extracellular matrix (ECM) of the diseased area such as tumor, inflammation, degeneration or for symptomatic objectives, or to injured smooth muscle cells, or for prevention. One kind of drug is RNA, as disclosed above, and this system may be used/and or adapted to the DNA targeting agent according to the invention as described herein, such as by means of example CRISPR Cas system of the present invention. The modes of implantation in some embodiments are existing implantation procedures that are developed and used today for other treatments, including brachytherapy and needle biopsy. In such cases the dimensions of the new implant described in this invention are similar to the original implant. Typically a few devices are implanted during the same treatment procedure.

As described in US Patent Publication 20110195123, there is provided a drug delivery implantable or insertable system, including systems applicable to a cavity such as the abdominal cavity and/or any other type of administration in which the drug delivery system is not anchored or attached, comprising a biostable and/or degradable and/or bioabsorbable polymeric substrate, which may for example optionally be a matrix. It should be noted that the term “insertion” also includes implantation. The drug delivery system is preferably implemented as a “Loder” as described in US Patent Publication 20110195123.

The polymer or plurality of polymers are biocompatible, incorporating an agent and/or plurality of agents, enabling the release of agent at a controlled rate, wherein the total volume of the polymeric substrate, such as a matrix for example, in some embodiments is optionally and preferably no greater than a maximum volume that permits a therapeutic level of the agent to be reached. As a non-limiting example, such a volume is preferably within the range of 0.1 m³ to 1000 mm³, as required by the volume for the agent load. The Loder may optionally be larger, for example when incorporated with a device whose size is determined by functionality, for example and without limitation, a knee joint, an intra-uterine or cervical ring and the like.

The drug delivery system (for delivering the composition) is designed in some embodiments to preferably employ degradable polymers, wherein the main release mechanism is bulk erosion; or in some embodiments, non degradable, or slowly degraded polymers are used, wherein the main release mechanism is diffusion rather than bulk erosion, so that the outer part functions as membrane, and its internal part functions as a drug reservoir, which practically is not affected by the surroundings for an extended period (for example from about a week to about a few months). Combinations of different polymers with different release mechanisms may also optionally be used. The concentration gradient at the surface is preferably maintained effectively constant during a significant period of the total drug releasing period, and therefore the diffusion rate is effectively constant (termed “zero mode” diffusion). By the term “constant” it is meant a diffusion rate that is preferably maintained above the lower threshold of therapeutic effectiveness, but which may still optionally feature an initial burst and/or may fluctuate, for example increasing and decreasing to a certain degree. The diffusion rate is preferably so maintained for a prolonged period, and it can be considered constant to a certain level to optimize the therapeutically effective period, for example the effective silencing period.

The drug delivery system optionally and preferably is designed to shield the nucleotide based therapeutic agent from degradation, whether chemical in nature or due to attack from enzymes and other factors in the body of the subject.

The drug delivery system as described in US Patent Publication 20110195123 is optionally associated with sensing and/or activation appliances that are operated at and/or after implantation of the device, by non and/or minimally invasive methods of activation and/or acceleration/deceleration, for example optionally including but not limited to thermal heating and cooling, laser beams, and ultrasonic, including focused ultrasound and/or RF (radiofrequency) methods or devices.

According to some embodiments of US Patent Publication 20110195123, the site for local delivery may optionally include target sites characterized by high abnormal proliferation of cells, and suppressed apoptosis, including tumors, active and or chronic inflammation and infection including autoimmune diseases states, degenerating tissue including muscle and nervous tissue, chronic pain, degenerative sites, and location of bone fractures and other wound locations for enhancement of regeneration of tissue, and injured cardiac, smooth and striated muscle.

The site for implantation of the composition, or target site, preferably features a radius, area and/or volume that is sufficiently small for targeted local delivery. For example, the target site optionally has a diameter in a range of from about 0.1 mm to about 5 cm.

The location of the target site is preferably selected for maximum therapeutic efficacy. For example, the composition of the drug delivery system (optionally with a device for implantation as described above) is optionally and preferably implanted within or in the proximity of a tumor environment, or the blood supply associated thereof.

For example the composition (optionally with the device) is optionally implanted within or in the proximity to pancreas, prostate, breast, liver, via the nipple, within the vascular system and so forth.

The target location is optionally selected from the group consisting of (as non-limiting examples only, as optionally any site within the body may be suitable for implanting a Loder): 1. brain at degenerative sites like in Parkinson or Alzheimer disease at the basal ganglia, white and gray matter; 2. spine as in the case of amyotrophic lateral sclerosis (ALS); 3. uterine cervix to prevent HPV infection; 4. active and chronic inflammatory joints; 5. dermis as in the case of psoriasis; 6. sympathetic and sensoric nervous sites for analgesic effect; 7. Intra osseous implantation; 8. acute and chronic infection sites; 9. Intra vaginal; 10. Inner ear—auditory system, labyrinth of the inner ear, vestibular system; 11. Intra tracheal; 12. Intra-cardiac; coronary, epicardiac; 13. urinary bladder; 14. biliary system; 15. parenchymal tissue including and not limited to the kidney, liver, spleen; 16. lymph nodes; 17. salivary glands; 18. dental gums; 19. Intra-articular (into joints); 20. Intra-ocular; 21. Brain tissue; 22. Brain ventricles; 23, Cavities, including abdominal cavity (for example but without limitation, for ovary cancer); 24. Intra esophageal and 25. Intra rectal.

Optionally insertion of the system (for example a device containing the composition) is associated with injection of material to the ECM at the target site and the vicinity of that site to affect local pH and/or temperature and/or other biological factors affecting the diffusion of the drug and/or drug kinetics in the ECM, of the target site and the vicinity of such a site.

Optionally, according to some embodiments, the release of said agent could be associated with sensing and/or activation appliances that are operated prior and/or at and/or after insertion, by non and/or minimally invasive and/or else methods of activation and/or acceleration/deceleration, including laser beam, radiation, thermal heating and cooling, and ultrasonic, including focused ultrasound and/or RF (radiofrequency) methods or devices, and chemical activators.

According to other embodiments of US Patent Publication 20110195123, the drug preferably comprises a RNA, for example for localized cancer cases in breast, pancreas, brain, kidney, bladder, lung, and prostate as described below. Although exemplified with RNAi, many drugs are applicable to be encapsulated in Loder, and can be used in association with this invention, as long as such drugs can be encapsulated with the Loder substrate, such as a matrix for example, and this system may be used and/or adapted to deliver the CRISPR Cas system of the present invention.

As another example of a specific application, neuro and muscular degenerative diseases develop due to abnormal gene expression. Local delivery of RNAs may have therapeutic properties for interfering with such abnormal gene expression. Local delivery of anti apoptotic, anti inflammatory and anti degenerative drugs including small drugs and macromolecules may also optionally be therapeutic. In such cases the Loder is applied for prolonged release at constant rate and/or through a dedicated device that is implanted separately. All of this may be used and/or adapted to the DNA targeting agent according to the invention as described herein, such as by means of example CRISPR Cas system of the present invention.

As yet another example of a specific application, psychiatric and cognitive disorders are treated with gene modifiers. Gene knockdown is a treatment option. Loders locally delivering agents to central nervous system sites are therapeutic options for psychiatric and cognitive disorders including but not limited to psychosis, bi-polar diseases, neurotic disorders and behavioral maladies. The Loders could also deliver locally drugs including small drugs and macromolecules upon implantation at specific brain sites. All of this may be used and/or adapted to the CRISPR Cas system of the present invention.

As another example of a specific application, silencing of innate and/or adaptive immune mediators at local sites enables the prevention of organ transplant rejection. Local delivery of RNAs and immunomodulating reagents with the Loder implanted into the transplanted organ and/or the implanted site renders local immune suppression by repelling immune cells such as CD8 activated against the transplanted organ. All of this may be used/and or adapted to the DNA targeting agent according to the invention as described herein, such as by means of example CRISPR Cas system of the present invention.

As another example of a specific application, vascular growth factors including VEGFs and angiogenin and others are essential for neovascularization. Local delivery of the factors, peptides, peptidomimetics, or suppressing their repressors is an important therapeutic modality; silencing the repressors and local delivery of the factors, peptides, macromolecules and small drugs stimulating angiogenesis with the Loder is therapeutic for peripheral, systemic and cardiac vascular disease.

The method of insertion, such as implantation, may optionally already be used for other types of tissue implantation and/or for insertions and/or for sampling tissues, optionally without modifications, or alternatively optionally only with non-major modifications in such methods. Such methods optionally include but are not limited to brachytherapy methods, biopsy, endoscopy with and/or without ultrasound, such as ERCP, stereotactic methods into the brain tissue, Laparoscopy, including implantation with a laparoscope into joints, abdominal organs, the bladder wall and body cavities.

Implantable device technology herein discussed can be employed with herein teachings and hence by this disclosure and the knowledge in the art, the DNA targeting agent according to the invention as described herein, such as by means of example CRISPR-Cas system or components thereof or nucleic acid molecules thereof or encoding or providing components may be delivered via an implantable device.

The present application also contemplates an inducible CRISPR Cas system. Reference is made to international patent application Serial No. PCT/US13/51418 filed Jul. 21, 2013, which published as WO2014/018423 on Jan. 30, 2014.

In one aspect the invention provides a DNA targeting agent according to the invention as described herein, such as by means of example a non-naturally occurring or engineered CRISPR Cas system which may comprise at least one switch wherein the activity of said CRISPR Cas system is controlled by contact with at least one inducer energy source as to the switch. In an embodiment of the invention the control as to the at least one switch or the activity of said CRISPR Cas system may be activated, enhanced, terminated or repressed. The contact with the at least one inducer energy source may result in a first effect and a second effect.

The first effect may be one or more of nuclear import, nuclear export, recruitment of a secondary component (such as an effector molecule), conformational change (of protein, DNA or RNA), cleavage, release of cargo (such as a caged molecule or a co-factor), association or dissociation. The second effect may be one or more of activation, enhancement, termination or repression of the control as to the at least one switch or the activity of said the DNA targeting agent according to the invention as described herein, such as by means of example CRISPR Cas system. In one embodiment the first effect and the second effect may occur in a cascade.

The invention comprehends that the inducer energy source may be heat, ultrasound, electromagnetic energy or chemical. In a preferred embodiment of the invention, the inducer energy source may be an antibiotic, a small molecule, a hormone, a hormone derivative, a steroid or a steroid derivative. In a more preferred embodiment, the inducer energy source maybe abscisic acid (ABA), doxycycline (DOX), cumate, rapamycin, 4-hydroxytamoxifen (4OHT), estrogen or ecdysone.

The invention provides that the at least one switch may be selected from the group consisting of antibiotic based inducible systems, electromagnetic energy based inducible systems, small molecule based inducible systems, nuclear receptor based inducible systems and hormone based inducible systems. In a more preferred embodiment the at least one switch may be selected from the group consisting of tetracycline (Tet)/DOX inducible systems, light inducible systems, ABA inducible systems, cumate repressor/operator systems, 4OHT/estrogen inducible systems, ecdysone-based inducible systems and FKBP12/FRAP (FKBP12-rapamycin complex) inducible systems.

In one aspect of the invention the inducer energy source is electromagnetic energy.

The electromagnetic energy may be a component of visible light having a wavelength in the range of 450 nm-700 nm. In a preferred embodiment the component of visible light may have a wavelength in the range of 450 nm-500 nm and may be blue light. The blue light may have an intensity of at least 0.2 mW/cm2, or more preferably at least 4 mW/cm2. In another embodiment, the component of visible light may have a wavelength in the range of 620-700 nm and is red light.

In a further aspect, the invention provides a method of controlling a the DNA targeting agent according to the invention as described herein, such as by means of example a non-naturally occurring or engineered CRISPR Cas system, comprising providing said CRISPR Cas system comprising at least one switch wherein the activity of said CRISPR Cas system is controlled by contact with at least one inducer energy source as to the switch.

In an embodiment of the invention, the invention provides methods wherein the control as to the at least one switch or the activity of said the DNA targeting agent according to the invention as described herein, such as by means of example CRISPR Cas system may be activated, enhanced, terminated or repressed. The contact with the at least one inducer energy source may result in a first effect and a second effect. The first effect may be one or more of nuclear import, nuclear export, recruitment of a secondary component (such as an effector molecule), conformational change (of protein, DNA or RNA), cleavage, release of cargo (such as a caged molecule or a co-factor), association or dissociation. The second effect may be one or more of activation, enhancement, termination or repression of the control as to the at least one switch or the activity of said CRISPR Cas system. In one embodiment the first effect and the second effect may occur in a cascade.

The invention comprehends that the inducer energy source may be heat, ultrasound, electromagnetic energy or chemical. In a preferred embodiment of the invention, the inducer energy source may be an antibiotic, a small molecule, a hormone, a hormone derivative, a steroid or a steroid derivative. In a more preferred embodiment, the inducer energy source maybe abscisic acid (ABA), doxycycline (DOX), cumate, rapamycin, 4-hydroxytamoxifen (4OHT), estrogen or ecdysone. The invention provides that the at least one switch may be selected from the group consisting of antibiotic based inducible systems, electromagnetic energy based inducible systems, small molecule based inducible systems, nuclear receptor based inducible systems and hormone based inducible systems. In a more preferred embodiment the at least one switch may be selected from the group consisting of tetracycline (Tet)/DOX inducible systems, light inducible systems. ABA inducible systems, cumate repressor/operator systems, 4OHT/estrogen inducible systems, ecdysone-based inducible systems and FKBP12/FRAP (FKBP12-rapamycin complex) inducible systems.

In one aspect of the methods of the invention the inducer energy source is electromagnetic energy. The electromagnetic energy may be a component of visible light having a wavelength in the range of 450 nm-700 nm. In a preferred embodiment the component of visible light may have a wavelength in the range of 450 nm-500 nm and may be blue light. The blue light may have an intensity of at least 0.2 mW/cm2, or more preferably at least 4 mW/cm2. In another embodiment, the component of visible light may have a wavelength in the range of 620-700 nm and is red light.

In another preferred embodiment of the invention, the inducible effector may be a Light Inducible Transcriptional Effector (LITE). The modularity of the LITE system allows for any number of effector domains to be employed for transcriptional modulation. In yet another preferred embodiment of the invention, the inducible effector may be a chemical. The invention also contemplates an inducible multiplex genome engineering using CRISPR (clustered regularly interspaced short palindromic repeats)/Cas systems.

Self-Inactivating Systems

Once all copies of a gene in the genome of a cell have been edited, continued CRISRP/Cas9 expression in that cell is no longer necessary. Indeed, sustained expression would be undesirable in case of off-target effects at unintended genomic sites, etc. Thus time-limited expression would be useful. Inducible expression offers one approach, but in addition Applicants have engineered a Self-Inactivating CRISPR-Cas9 system that relies on the use of a non-coding guide target sequence within the CRISPR vector itself. Thus, after expression begins, the CRISPR system will lead to its own destruction, but before destruction is complete it will have time to edit the genomic copies of the target gene (which, with a normal point mutation in a diploid cell, requires at most two edits). Simply, the self inactivating CRISPR-Cas system includes additional RNA (i.e., guide RNA) that targets the coding sequence for the CRISPR enzyme itself or that targets one or more non-coding guide target sequences complementary to unique sequences present in one or more of the following:

(a) within the promoter driving expression of the non-coding RNA elements,
(b) within the promoter driving expression of the Cas9 gene,
(c) within 100 bp of the ATG translational start codon in the Cas9 coding sequence,
(d) within the inverted terminal repeat (iTR) of a viral delivery vector, e.g., in the AAV genome.

Furthermore, that RNA can be delivered via a vector, e.g., a separate vector or the same vector that is encoding the CRISPR complex. When provided by a separate vector, the CRISPR RNA that targets Cas expression can be administered sequentially or simultaneously. When administered sequentially, the CRISPR RNA that targets Cas expression is to be delivered after the CRISPR RNA that is intended for e.g. gene editing or gene engineering. This period may be a period of minutes (e.g. 5 minutes, 10 minutes, 20 minutes, 30 minutes, 45 minutes, 60 minutes). This period may be a period of hours (e.g. 2 hours, 4 hours, 6 hours, 8 hours, 12 hours, 24 hours). This period may be a period of days (e.g. 2 days, 3 days, 4 days, 7 days). This period may be a period of weeks (e.g. 2 weeks, 3 weeks, 4 weeks). This period may be a period of months (e.g. 2 months, 4 months, 8 months, 12 months). This period may be a period of years (2 years, 3 years, 4 years). In this fashion, the Cas enzyme associates with a first gRNA/chiRNA capable of hybridizing to a first target, such as a genomic locus or loci of interest and undertakes the function(s) desired of the CRISPR-Cas system (e.g., gene engineering); and subsequently the Cas enzyme may then associate with the second gRNA/chiRNA capable of hybridizing to the sequence comprising at least part of the Cas or CRISPR cassette. Where the gRNA/chiRNA targets the sequences encoding expression of the Cas protein, the enzyme becomes impeded and the system becomes self inactivating. In the same manner. CRISPR RNA that targets Cas expression applied via, for example liposome, lipofection, nanoparticles, microvesicles as explained herein, may be administered sequentially or simultaneously. Similarly, self-inactivation may be used for inactivation of one or more guide RNA used to target one or more targets.

In some aspects, a single gRNA is provided that is capable of hybridization to a sequence downstream of a CRISPR enzyme start codon, whereby after a period of time there is a loss of the CRISPR enzyme expression. In some aspects, one or more gRNA(s) are provided that are capable of hybridization to one or more coding or non-coding regions of the polynucleotide encoding the CRISPR-Cas system, whereby after a period of time there is a inactivation of one or more, or in some cases all, of the CRISPR-Cas system. In some aspects of the system, and not to be limited by theory, the cell may comprise a plurality of CRISPR-Cas complexes, wherein a first subset of CRISPR complexes comprise a first chiRNA capable of targeting a genomic locus or loci to be edited, and a second subset of CRISPR complexes comprise at least one second chiRNA capable of targeting the polynucleotide encoding the CRISPR-Cas system, wherein the first subset of CRISPR-Cas complexes mediate editing of the targeted genomic locus or loci and the second subset of CRISPR complexes eventually inactivate the CRISPR-Cas system, thereby inactivating further CRISPR-Cas expression in the cell.

Thus the invention provides a CRISPR-Cas system comprising one or more vectors for delivery to a eukaryotic cell, wherein the vector(s) encode(s): (i) a CRISPR enzyme; (ii) a first guide RNA capable of hybridizing to a target sequence in the cell; (iii) a second guide RNA capable of hybridizing to one or more target sequence(s) in the vector which encodes the CRISPR enzyme; (iv) at least one tracr mate sequence; and (v) at least one tracr sequence, The first and second complexes can use the same tracr and tracr mate, thus differing only by the guide sequence, wherein, when expressed within the cell: the first guide RNA directs sequence-specific binding of a first CRISPR complex to the target sequence in the cell; the second guide RNA directs sequence-specific binding of a second CRISPR complex to the target sequence in the vector which encodes the CRISPR enzyme; the CRISPR complexes comprise (a) a tracr mate sequence hybridised to a tracr sequence and (b) a CRISPR enzyme bound to a guide RNA, such that a guide RNA can hybridize to its target sequence; and the second CRISPR complex inactivates the CRISPR-Cas system to prevent continued expression of the CRISPR enzyme by the cell.

Further characteristics of the vector(s), the encoded enzyme, the guide sequences, etc. are disclosed elsewhere herein. For instance, one or both of the guide sequence(s) can be part of a chiRNA sequence which provides the guide, tracr mate and tracr sequences within a single RNA, such that the system can encode (i) a CRISPR enzyme; (ii) a first chiRNA comprising a sequence capable of hybridizing to a first target sequence in the cell, a first tracr mate sequence, and a first tracr sequence; (iii) a second guide RNA capable of hybridizing to the vector which encodes the CRISPR enzyme, a second tracr mate sequence, and a second tracr sequence. Similarly, the enzyme can include one or more NLS, etc.

The various coding sequences (CRISPR enzyme, guide RNAs, tracr and tracr mate) can be included on a single vector or on multiple vectors. For instance, it is possible to encode the enzyme on one vector and the various RNA sequences on another vector, or to encode the enzyme and one chiRNA on one vector, and the remaining chiRNA on another vector, or any other permutation. In general, a system using a total of one or two different vectors is preferred.

Where multiple vectors are used, it is possible to deliver them in unequal numbers, and ideally with an excess of a vector which encodes the first guide RNA relative to the second guide RNA, thereby assisting in delaying final inactivation of the CRISPR system until genome editing has had a chance to occur.

The first guide RNA can target any target sequence of interest within a genome, as described elsewhere herein. The second guide RNA targets a sequence within the vector which encodes the CRISPR Cas9 enzyme, and thereby inactivates the enzyme's expression from that vector. Thus the target sequence in the vector must be capable of inactivating expression. Suitable target sequences can be, for instance, near to or within the translational start codon for the Cas9 coding sequence, in a non-coding sequence in the promoter driving expression of the non-coding RNA elements, within the promoter driving expression of the Cas9 gene, within 100 bp of the ATG translational start codon in the Cas9 coding sequence, and/or within the inverted terminal repeat (iTR) of a viral delivery vector, e.g., in the AAV genome. A double stranded break near this region can induce a frame shift in the Cas9 coding sequence, causing a loss of protein expression. An alternative target sequence for the “self-inactivating” guide RNA would aim to edit/inactivate regulatory regions/sequences needed for the expression of the CRISPR-Cas9 system or for the stability of the vector. For instance, if the promoter for the Cas9 coding sequence is disrupted then transcription can be inhibited or prevented. Similarly, if a vector includes sequences for replication, maintenance or stability then it is possible to target these. For instance, in a AAV vector a useful target sequence is within the iTR. Other useful sequences to target can be promoter sequences, polyadenylation sites, etc.

Furthermore, if the guide RNAs are expressed in array format, the “self-inactivating” guide RNAs that target both promoters simultaneously will result in the excision of the intervening nucleotides from within the CRISPR-Cas expression construct, effectively leading to its complete inactivation. Similarly, excision of the intervening nucleotides will result where the guide RNAs target both ITRs, or targets two or more other CRISPR-Cas components simultaneously. Self-inactivation as explained herein is applicable, in general, with CRISPR-Cas9 systems in order to provide regulation of the CRISPR-Cas9. For example, self-inactivation as explained herein may be applied to the CRISPR repair of mutations, for example expansion disorders, as explained herein. As a result of this self-inactivation, CRISPR repair is only transiently active.

Addition of non-targeting nucleotides to the 5′ end (e.g. 1-10 nucleotides, preferably 1-5 nucleotides) of the “self-inactivating” guide RNA can be used to delay its processing and/or modify its efficiency as a means of ensuring editing at the targeted genomic locus prior to CRISPR-Cas9 shutdown.

In one aspect of the self-inactivating AAV-CRISPR-Cas9 system, plasmids that co-express one or more sgRNA targeting genomic sequences of interest (e.g. 1-2, 1-5, 1-10, 1-15, 1-20, 1-30) may be established with “self-inactivating” sgRNAs that target an SpCas9 sequence at or near the engineered ATG start site (e.g. within 5 nucleotides, within 15 nucleotides, within 30 nucleotides, within 50 nucleotides, within 100 nucleotides). A regulatory sequence in the U6 promoter region can also be targeted with an sgRNA. The U6-driven sgRNAs may be designed in an array format such that multiple sgRNA sequences can be simultaneously released. When first delivered into target tissue/cells (left cell) sgRNAs begin to accumulate while Cas9 levels rise in the nucleus. Cas9 complexes with all of the sgRNAs to mediate genome editing and self-inactivation of the CRISPR-Cas9 plasmids.

One aspect of a self-inactivating CRISPR-Cas9 system is expression of singly or in tandem array format from 1 up to 4 or more different guide sequences; e.g. up to about 20 or about 30 guides sequences. Each individual self inactivating guide sequence may target a different target. Such may be processed from, e.g. one chimeric pol3 transcript. Pol3 promoters such as U6 or H1 promoters may be used. Pol2 promoters such as those mentioned throughout herein. Inverted terminal repeat (iTR) sequences may flank the Pol3 promoter-sgRNA(s)-Pol2 promoter-Cas9.

One aspect of a chimeric, tandem array transcript is that one or more guide(s) edit the one or more target(s) while one or more self inactivating guides inactivate the CRISPR/Cas9 system. Thus, for example, the described CRISPR-Cas9 system for repairing expansion disorders may be directly combined with the self-inactivating CRISPR-Cas9 system described herein. Such a system may, for example, have two guides directed to the target region for repair as well as at least a third guide directed to self-inactivation of the CRISPR-Cas9. Reference is made to Application Ser. No. PCT/US2014/069897, entitled “Compositions And Methods Of Use Of Crispr-Cas Systems In Nucleotide Repeat Disorders.” published Dec. 12, 2014 as WO/2015/089351.

One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887: Kim. Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms.

In advantageous embodiments of the invention, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), monomers with an RVD of NG preferentially bind to thymine (T), monomers with an RVD of HD preferentially bind to cytosine (C) and monomers with an RVD of NN preferentially bind to both adenine (A) and guanine (G). In yet another embodiment of the invention, monomers with an RVD of IG preferentially bind to T. Thus, the number and order of the polypeptide monomer repeats in the nucleic acid binding domain of a TALE determines its nucleic acid target specificity. In still further embodiments of the invention, monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009), and Zhang et al., Nature Biotechnology 29:149-153 (2011), each of which is incorporated by reference in its entirety.

The polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH. KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, monomers having RVDs of H*, HA, KA, N*, NA, NC, NS, RA, and S* bind to adenine, guanine, cytosine and thymine with comparable affinity.

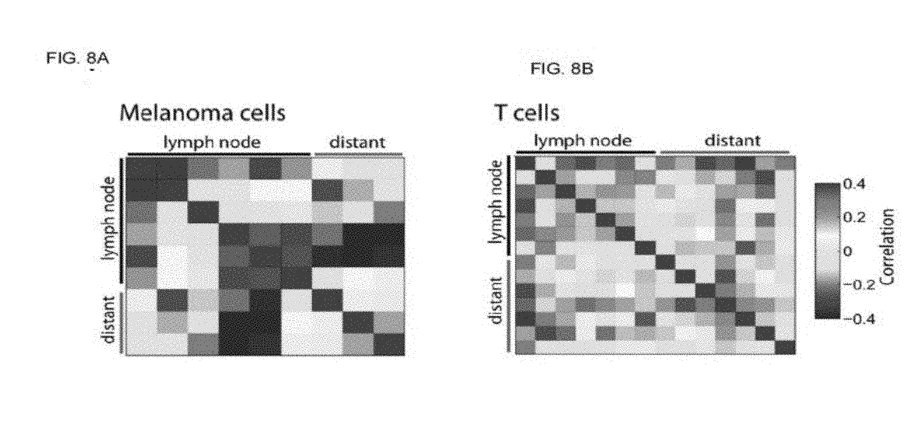

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and polypeptides of the invention may target DNA sequences that begin with T, A. G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8). Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 18)
M D P I R S R T P S P A R E L L S G P Q P D G V Q
P T A D R G V S P P A G G P L D G L P A R R T M S
R T R L P S P P A P S P A P S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplar) amino acid sequence of a C-terminal capping region is:

(SEQ ID NO: 19)
R P A L E S I V A Q L S R P D P A L A A L T N D H
L V A L A C L G G R P A L D A V K K G L P H A P A
L I K R T N R R I P E R I S H R V A D H A Q V V R
V F G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an m Sin interaction domain (SID). SID4X domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4: and Dudley et al., (2005) Journal of Clinical Oncology 23 (10): 2346-57.) or genetically re-directed peripheral blood mononuclear cells (Johnson et al., (2009) Blood 114 (3): 535-46: and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73).

Aspects of the invention involve the adoptive transfer of immune system cells, such as T cells, specific for selected antigens, such as tumor associated antigens (see Maus et al., 2014, Adoptive Immunotherapy for Cancer or Viruses, Annual Review of Immunology, Vol. 32: 189-225: Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144). Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962. WO2013166321, WO2013039889, WO2014018863, WO2014083173: U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322). Alternative CAR constructs may be characterized as belonging to successive generations. First-generation CARs typically consist of a single-chain variable fragment of an antibody specific for an antigen, for example comprising a V_{L} linked to a V_{H} of a specific antibody, linked by a flexible linker, for example by a CD8a hinge domain and a CD8a transmembrane domain, to the transmembrane and intracellular signaling domains of either CD3C or FcRγ (scFv-CD3′ or scFv-FcRγ; see U.S. Pat. No. 7,741,465; U.S. Pat. No. 5,912,172; U.S. Pat. No. 5,906,936). Second-generation CARs incorporate the intracellular domains of one or more costimulatory molecules, such as CD28, OX40 (CD134). or 4-1BB (CD137) within the endodomain (for example scFv-CD28/OX40/4-1BB-CD3ζ; see U.S. Pat. Nos. 8,911,993; 8,916,381; 8,975,071; 9,101,584: 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, or CD28 signaling domains (for example scFv-CD28-4-1 BB-CD3′ or scFv-CD28-OX40-CD3ζ; see U.S. Pat. No. 8,906,682; U.S. Pat. No. 8,399,645; U.S. Pat. No. 5,686,281: PCT Publication No. WO2014134165: PCT Publication No. WO2012079000). Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203: 7,160,682; 7,985,739; 8,227,432), may be used to introduce CARs, for example using 2nd generation antigen-specific CARs signaling through CD3C and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to threat tumor xenografts.

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoreponsive cell, thereby treating or preventing the disease (such as a neoplasia, a pathogen infection, an autoimmune disorder, or an allogeneic transplant reaction).

In one embodiment, the treatment can be administrated into patients undergoing an immunosuppressive treatment. The cells or population of cells, may be made resistant to at least one immunosuppressive agent due to the inactivation of a gene encoding a receptor for such immunosuppressive agent. Not being bound by a theory, the immunosuppressive treatment should help the selection and expansion of the immunoresponsive or T cells according to the invention within the patient.

The administration of the cells or population of cells according to the present invention may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905: Di Stasi et al., The New England Journal of Medicine 2011; 365:1673-1683: Sadelain M, The New England Journal of Medicine 2011; 365:1735-173: Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed to eliminate potential alloreactive T-cell receptors (TCR), disrupt the target of a chemotherapeutic agent, block an immune checkpoint, activate a T cell, and/or increase the differentiation and/or proliferation of functionally exhausted or dysfunctional CD8+ T-cells (see PCT Patent Publications: WO2013176915, WO2014059173. WO2014172606, WO2014184744, and WO2014191128). Editing may result in inactivation of a gene.

By inactivating a gene it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However. NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen. T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT1 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1 or TIM-3. In preferred embodiments, the gene locus involved in the expression of PD-1 or CTLA-4 genes is targeted. In other preferred embodiments, combinations of genes are targeted, such as but not limited to PD-1 and TIGIT.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα. LAIR1 and TCRβ, SIGLEC10 and TCRα, SIGLEC10 and TCRβ, 2B4 and TCRα, 2B4 and TCRβ.

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Cell therapy methods often involve the ex-vivo activation and expansion of T-cells. In one embodiment T cells are activated before administering them to a subject in need thereof. Activation or stimulation methods have been described herein and is preferably required before T cells are administered to a subject in need thereof. Examples of these type of treatments include the use tumor infiltrating lymphocyte (TIL) cells (see U.S. Pat. No. 5,126,132), cytotoxic T-cells (see U.S. Pat. No. 6,255,073, and U.S. Pat. No. 5,846,827), expanded tumor draining lymph node cells (see U.S. Pat. No. 6,251,385), and various other lymphocyte preparations (see U.S. Pat. No. 6,194,207; U.S. Pat. No. 5,443,983; U.S. Pat. No. 6,040,177: and U.S. Pat. No. 5,766,920). These patents are herein incorporated by reference in their entirety.

For maximum effectiveness of T-cells in cell therapy protocols, the ex vivo activated T-cell population should be in a state that can maximally orchestrate an immune response to cancer, infectious diseases, or other disease states. For an effective T-cell response, the T-cells first must be activated. For activation, at least two signals are required to be delivered to the T-cells. The first signal is normally delivered through the T-cell receptor (TCR) on the T-cell surface. The TCR first signal is normally triggered upon interaction of the TCR with peptide antigens expressed in conjunction with an MHC complex on the surface of an antigen-presenting cell (APC). The second signal is normally delivered through co-stimulatory receptors on the surface of T-cells. Co-stimulatory receptors are generally triggered by corresponding ligands or cytokines expressed on the surface of APCs.

Due to the difficulty in maintaining large numbers of natural APC in cultures of T-cells being prepared for use in cell therapy protocols, alternative methods have been sought for ex-vivo activation of T-cells. One method is to by-pass the need for the peptide-MHC complex on natural APCs by instead stimulating the TCR (first signal) with polyclonal activators, such as immobilized or cross-linked anti-CD3 or anti-CD2 monoclonal antibodies (mAbs) or superantigens. The most investigated co-stimulatory agent (second signal) used in conjunction with anti-CD3 or anti-CD2 mAbs has been the use of immobilized or soluble anti-CD28 mAbs. The combination of anti-CD3 mAb (first signal) and anti-CD28 mAb (second signal) immobilized on a solid support such as paramagnetic beads (see U.S. Pat. No. 6,352,694, herein incorporated by reference in its entirety) has been used to substitute for natural APCs in inducing ex-vivo T-cell activation in cell therapy protocols (Levine. Bernstein et al., 1997 Journal of Immunology: 159:5921-5930: Garlie, LeFever et al., 1999 J Immunother. July; 22(4):336-45; Shibuya, Wei et al., 2000 Arch Otolaryngol Head Neck Surg. 126(4):473-9).

In one embodiment T cells that have infiltrated a tumor are isolated. T cells may be removed during surgery. T cells may be isolated after removal of tumor tissue by biopsy. T cells may be isolated by any means known in the art. In one embodiment the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Camivora, including Felines (cats) and Canines (dogs); the order Artiodactyla including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells, bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free. Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment. T cells are isolated by incubation with anti-CD3/anti-CD28 (i.e., 3×28)-conjugated beads, such as DYNABEADS®, M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

In one embodiment of the present invention, any combination of therapeutic, not limited to a small molecule, compound, mixture, nucleic acid, vector, or protein, is administered to a subject in order to increase or decrease the activity of the complement system. Exemplary embodiments for activation of complement are natural products such as snake venom and caterpillar bristles (PLoS Negl Trop Dis. 2013 Oct. 31:7(10):e2519: and PLoS One. 2015 Mar. 11:10(3):e0118615). Other molecules capable of activating complement have been described, such as C-reactive protein (CRP). Pharmaceutical grade CRP has been described previously (Circulation Research. 2014: 114: 672-676). Additionally, therapeutic antibodies may be used to activate or inhibit complement. In one embodiment, antibody drug conjugates may be used. In other embodiments, dual targeting compounds and/or antibodies may be used. Not being bound by a theory, a dual antibody may bind complement in one aspect and, for example, a tumor in another aspect, so as to localize the complement to a tumor. An antibody of the present invention may be an antibody fragment. The antibody fragment may be a nanobody, Fab, Fab′, (Fab′)2, Fv, ScFv, diabody, triabody, tetrabody, Bis-scFv, minibody, Fab2, or Fab3 fragment.

Inhibitors of the complement system are well known in the art and are useful for the practice of the present invention (see, e.g., Ricklin et al., Progress and trends in complement therapeutics. Adv Exp Med Biol. 2013:735:1-22.; Ricklin et al., Complement-targeted therapeutics. Nat Biotechnol. 2007 November; 25(11): 1265-1275; and Reis et al., Applying complement therapeutics to rare diseases. Clin Immunol. 2015 December; 161(2):225-40, herein incorporated by reference in their entirety).

A “complement inhibitor” is a molecule that prevents or reduces activation and/or propagation of the complement cascade that results in the formation of C3a or signaling through the C3a receptor, or C5a or signaling through the C5a receptor. A complement inhibitor can operate on one or more of the complement pathways, i.e., classical, alternative or lectin pathway. A “C3 inhibitor” is a molecule or substance that prevents or reduces the cleavage of C3 into C3a and C3b. A “C5a inhibitor” is a molecule or substance that prevents or reduces the activity of C5a. A “C5aR inhibitor” is a molecule or substance that prevents or reduces the binding of C5a to the C5a receptor. A “C3aR inhibitor” is a molecule or substance that prevents or reduces binding of C3a to the C3a receptor. A “factor D inhibitor” is a molecule or substance that prevents or reduces the activity of Factor D. A “factor B inhibitor” is a molecule or substance that prevents or reduces the activity of factor B. A “C4 inhibitor” is a molecule or substance that prevents or reduces the cleavage of C4 into C4b and C4a. A “C1q inhibitor” is a molecule or substance that prevents or reduces C1q binding to antibody-antigen complexes, virions, infected cells, or other molecules to which C1q binds to initiate complement activation. Any of the complement inhibitors described herein may comprise antibodies or antibody fragments, as would be understood by the person of skill in the art.

Antibodies useful in the present invention, such as antibodies that specifically bind to either C4, C3 or C5 and prevent cleavage, or antibodies that specifically bind to factor D, factor B, C1q, or the C3a or C5a receptor, can be made by the skilled artisan using methods known in the art. Anti-C3 and anti-C5 antibodies are also commercially available.

A “complement activator” is a molecule that activates or increases activation and/or propagation of the complement cascade that results in the formation of C3a or signaling through the C3a receptor, or C5a or signaling through the C5a receptor. A complement activator can operate on one or more of the complement pathways, i.e., classical, alternative or lectin pathway.

Inhibitors or activators of the complement system may be administered by any known means in the art and by any means described herein. The inhibitors or activators may be targeted to a specific site of disease, such as, but not limited to a tumor. Monitoring by any means described herein may be used to determine if the therapy is effective. Such combination of a therapeutic targeting complement and monitoring provides advantages over any methods known in the art. Not being bound by a theory, the infiltration of cell populations, such as CAFs, T cells, macrophages, B cells may be monitored during treatment with an agent that activates or inhibits a component of the complement system. Not being bound by a theory a gene signature within a specific cell population as described herein may be monitored during treatment with an agent that activates or inhibits a component of the complement system. Not being bound by a theory, the present invention is provided by the Applicants discovery of cell specific gene expression signatures of cells within different cancers correlating to immune status, tumor status, and immune cell abundance. Moreover, applicants discovery of the correlation of complement gene expression in specific cell types to immune cell abundance allows for activating or inhibiting complement in order to modulate the microenvironment, including an immune response, for treatment of a disease. As illustrated by the examples, Applicants show that the expression of complement in relation to an immune response, and specifically, immune cell abundance is not limited to a specific cancer. Applicants provide data showing consistent gene expression patterns of complement components in single cells for melanoma, head and neck cancer, glioma, metastases to the brain, and across the TCGA tumors (see Examples). Not being bound by a theory, immune cell abundance is and gene expression signatures in single cells part of the microenvironment is a general phenomena that provides for activating and inhibiting complement in relation to many diseases and conditions, preferably cancer.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989): CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

The practice of the present invention employs, unless otherwise indicated, conventional techniques for generation of genetically modified mice. See Marten H. Hofker and Jan van Deursen, TRANSGENIC MOUSE METHODS AND PROTOCOLS, 2nd edition (2011).

These and other technologies may be employed in or as to the practice of the instant invention.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1 Methods for Melanoma

Tissue Handling and Tumor Disaggregation

Resected tumors were transported in DMEM (ThermoFisher Scientific) on ice immediately after surgical procurement. Tumors were rinsed with PBS (Life Technologies). A small fragment was stored in RNA-protect (Qiagen) for bulk RNA and DNA isolation. Using scalpels, the remainder of the tumor was minced into tiny cubes <1 mm3 and transferred into a 50 ml conical tube (BD Falcon) containing 10 ml pre-warmed M199-media (ThermoFisher Scientific), 2 mg/ml collagenase P (Roche) and 10 U/μl DNase I (Roche). Tumor pieces were digested in this digestion media for 10 minutes at 37° C., then vortexed for 10 seconds and pipetted up and down for 1 minute using pipettes of descending sizes (25 ml, 10 ml and 5 ml). If needed, this was repeated twice more until a single-cell suspension was obtained. This suspension was then filtered using a 70 μm nylon mesh (ThermoFisher Scientific) and residual cell clumps were discarded. The suspension was supplemented with 30 ml PBS (Life Technologies) with 2% fetal calf serum (FCS) (Gemini Bioproducts) and immediately placed on ice. After centrifuging at 580 g at 4° C. for 6 minutes, the supernatant was discarded and the cell pellet was re-suspended in PBS with FCS and placed on ice prior to staining for FACS. An ex vivo FNA was performed on Melanoma 80 using a 20G needle with a 10 ml syringe primed with 500 μl digestion media. The aspirate was incubated at 37 C for 10 minutes, filtered, spun down and supplemented with 10 ml PBS with FCS and immediately placed on ice and processed similar to the tissue samples as above.

Flow Cytometry

Single-cell suspensions were stained with CD45-FITC (VWR) and Calcein-AM (Life Technologies) per manufacturer recommendations. For sorting of ex vivo co-cultured cancer-associated fibroblasts, Applicants used a CD90-PE antibody (BioLegend). First, doublets were excluded based on forward and sideward scatter, then Applicants gated on viable cells (Calcein^{high}) and sorted single cells (CD45+ or CD45− or CD45− CD90+) into 96-well plates chilled to 4° C., pre-prepared with 10 μl TCL buffer (Qiagen) supplemented with 1% beta-mercaptoethanol (lysis buffer). Single-cell lysates were sealed, vortexed, spun down at 3700 rpm at 4° C. for 2 minutes, immediately placed on dry ice and transferred for storage at −80° C. Plates were thawed on ice prior to library construction and sequencing.

RNA/DNA Isolation from Bulk Specimens

RNA and DNA was isolated using the Qiagen minikit following the manufacturers recommendations.

Whole Transcriptome Amplification

Whole Transcriptome amplification (WTA) was performed with a modified SMART-Seq2 protocol, as described previously (50, 51), with Maxima Reverse Transcriptase (Life Technologies) used in place of Superscript II. Briefly, Applicants used Agencourt RNA-Clean streptavidin beads to precipitate nucleic acids, which were cleaned by washing with 70% ethanol and then primed for reverse transcription under the following conditions:

### Conditions I:

a) 72C, 3 min

After priming, reverse transcription was carried out with Maxima Reverse Transcription enzyme under the following cycling conditions: Initial step

a) 42C, 90 min

10 cycles

b) 50C, 2 min

c) 42C, 2 min

### Inactivation

d) 70C, 15 min

Following reverse transcription, the double stranded RT product was amplified by PCR with a Kapa Ready Mix under the following conditions: Initial step

a) 98C, 3 min

21 cycles

b) 98C, 15 sec

c) 67C, 20 sec

d) 72C, 6 min

### Extension

e) 72C, 5 min

Library Preparation and RNA-Seq

WTA products were cleaned with Agencourt XP DNA beads and 70% ethanol (Beckman Coulter) and Illumina sequencing libraries were prepared using Nextera XT (Illumina), as previously described (51). The 96 samples of a multiwall plate were pooled together, and cleaned with two 0.8×DNA SPRIs (Beckman Coulter). Library quality was assessed with a high sensitivity DNA chip (Agilent) and quantified with a high sensitivity dsDNA Quant Kit (Life Technologies). Samples were sequenced on an Illumina NextSeq 500 instrument using 30 bp paired-end reads.

Whole-Exome Sequencing and Analysis

Exome sequences were captured using Illumina technology and Exome sequence data processing and analysis were performed using the Picard and Firehose pipelines at the Broad Institute. The Picard pipeline (picard.sourceforge.net) was used to produce a BAM file with aligned reads. This includes alignment to the hg19 human reference sequence using the Burrows-Wheeler transform algorithm (52) and estimation of base quality score and recalibration with the Genome Analysis Toolkit (GATK) (www.broadinstitute.org/gatk/)(53). All sample pairs passed the Firehose pipeline including a QC pipeline to test for any tumor/normal and inter-individual contamination as previously described (54, 55). The MuTect algorithm was used to identify somatic mutations (55). MuTect identifies candidate somatic mutations by Bayesian statistical analysis of bases and their qualities in the tumor and normal BAMs at a given genomic locus. To reduce false positive calls Applicants additionally analyzed reads covering sites of an identified somatic mutation and realigned them with NovoAlign (www.novocraft.com) and performed additional iteration of MuTect inference on newly aligned BAM files. Furthermore, Applicants filtered somatic mutation calls using a panel of over 8,000 TCGA Normal samples. Small somatic insertions and deletions were detected using the Strelka algorithm (56) and similarly subjected to filtering out potential false positive using the panel of TCGA Normal samples. Somatic mutations including single-nucleotide variants, insertions, and deletions were annotated using Oncotator (57). Copy-ratios for each captured exon were calculated by comparing the mean exon coverage with expected coverage based on a panel of normal samples. The resulting copy ratio profiles were then segmented using the circular binary segmentation (CBS) algorithm (58).

Pre-Processing of RNA-Seq Data

Following sequencing, data is procured as a series of BAM files corresponding to each of the four lanes on the NextSeq and each of the paired ends and indices. BAM files were demultiplexed according to indices to distinguish single-cell samples from each other and converted to FASTQ files. The FASTQ files from all four lanes for a single sample were combined and the “left-hand” and “right-hand” read data of each read for each cell was aligned to UCSC Hg19. The alignment algorithm estimates alignment rate and gene expression levels were quantified by RSEM v. 1.12, producing a matrix of transcripts per million reads per gene for each cell.

Processing of RNA-Seq Data

Following sequencing on the NextSeq, BAM files were converted to merged, demultiplexed FASTQs. Paired-end reads were then mapped to the UCSC hg19 human transcriptome using Bowtie (59) with parameters “-q --phred33-quals -n 1-e 99999999-1 25-I 1-X 2000 -a -m 15 -S -p 6”, which allows alignment of sequences with single base changes such as due to point mutations. Expression levels of genes were quantified as Ei,j=log 2(TPMi,j/10+1), where TPMi,j refers to transcript-per-million (TPM) for gene i in sample j, as calculated by RSEM (60) v1.2.3 in paired-end mode. TPM values were divided by 10 since Applicants estimate the complexity of our single cell libraries to be on the order of 100,000 transcripts and would like to avoid counting each transcript ˜10 times, as would be the case with TPM, which may inflate the difference between the expression level of a gene in cells in which the gene is detected and those in which it is not detected. When evaluating the average expression of a population of cells by pooling data across cells (e.g., all cells from a given tumor or cell type) the division by 10 was not required and the average expression was defined Ep(I)=log 2(TPM(I)+1), where I is a set of cells.

For each cell, Applicants quantified the number of genes for which at least one read was mapped, and the average expression level of a curated list of housekeeping genes (Table 16). Applicants then excluded all cells with either fewer than 1,700 detected genes or an average housekeeping expression (E, as defined above) below 3. For the remaining cells, Applicants calculated the pooled expression of each gene as (Ep), and excluded genes with an aggregate expression below 4, which defined a different set of genes in different analyses depending on the subset of cells included. For the remaining cells and genes, Applicants defined relative expression by centering the expression levels, Eri,j=Ei,j-average[Ei, 1 . . . n].

TABLE 16
Curated list of housekeeping genes
used for quality control analysis.

ACTB
B2M
HNRPLL
HPRT
PSMB2
PSMB4
PPIA
PRPS1
PRPS1L1
PRPS1L3
PRPS2
PRPSAP1
PRPSAP2
RPL10
RPL10A
RPL10L
RPL11
RPL12
RPL13
RPL14
RPL15
RPL17
RPL18
RPL19
RPL21
RPL22
RPL22L1
RPL23
RPL24
RPL26
RPL27
RPL28
RPL29
RPL3
RPL30
RPL32
RPL34
RPL35
RPL36
RPL37
RPL38
RPL39L
RPL3L
RPL4
RPL41
RPL5
RPL6
RPL7
RPL7A
RPL7L1
RPL8
RPL9
RPLP0
RPLP1
RPLP2
RPS10
RPS11
RPS12
RPS13
RPS14
RPS15
RPS15A
RPS16
RPS17
RPS18
RPS19
RPS20
RPS21
RPS24
RPS25
RPS26
RPS27
RPS27A
RPS27L
RPS28
RPS29
RPS3
RPS3A
RPS4X
RPS5
RPS6
RPS6KA1
RPS6KA2
RPS6KA3
RPS6KA4
RPS6KA5
RPS6KA6
RPS6KB1
RPS6KB2
RPS6KC1
RPS6KL1
RPS7
RPS8
RPS9
RPSA
TRPS1
UBB

Data Availability

Raw and processed single-cell RNA-seq data is available through the Gene Expression Omnibus (GSE72056).

CNV Estimation

Initial CNVs (CNV0) were estimated by sorting the analyzed genes by their chromosomal location and applying a moving average to the relative expression values, with a sliding window of 100 genes within each chromosome, as previously described (15). To avoid considerable impact of any particular gene on the moving average Applicants limited the relative expression values to [−3,3] by replacing all values above 3 by 3, and replacing values below −3 by −3. This was performed only in the context of CNV estimation. This initial analysis is based on the average expression of genes in each cell compared to all other cells and therefore does not have a proper reference which is required to define the baseline. However, Applicants identified five subsets of cells that each had more limited high or low values of CNV0 and which were consistent across the genome despite the fact that these cells originate from multiple tumors. Applicants thus considered these as putative non-malignant cells and used their CNV estimates to define the baseline. The normal cells included five cell types (see below, not including NK cells), which differed in gene expression patterns and accordingly also slightly in CNV estimates (e.g., the MHC region in chromosome 6 had consistently higher values in T cells than in stromal or cancer cells). Applicants therefore defined multiple baselines, as the average of each cell type, and based on these the maximal (BaseMax) and minimal (BaseMin) baseline at each window of 100 genes. The final CNV estimate of cell i at position j was then defined as:

$${CNV}_{f} ( i , j ) = \{ \begin{matrix}{CNV}_{0} ( i , j ) - BaseMax ( j ) , if {CNV}_{0} ( i , j ) > BaseMax ( j ) + 0.2 \\ {CNV}_{0} ( i , j ) - BaseMin ( j ) , if {CNV}_{0} ( i , j ) < BaseMin ( j ) - 0.2 \\ 0 , if BaseMin ( j ) - 0.2 < {CNV}_{0} ( i , j ) < BaseMin ( j ) + 0.2\end{matrix}$$

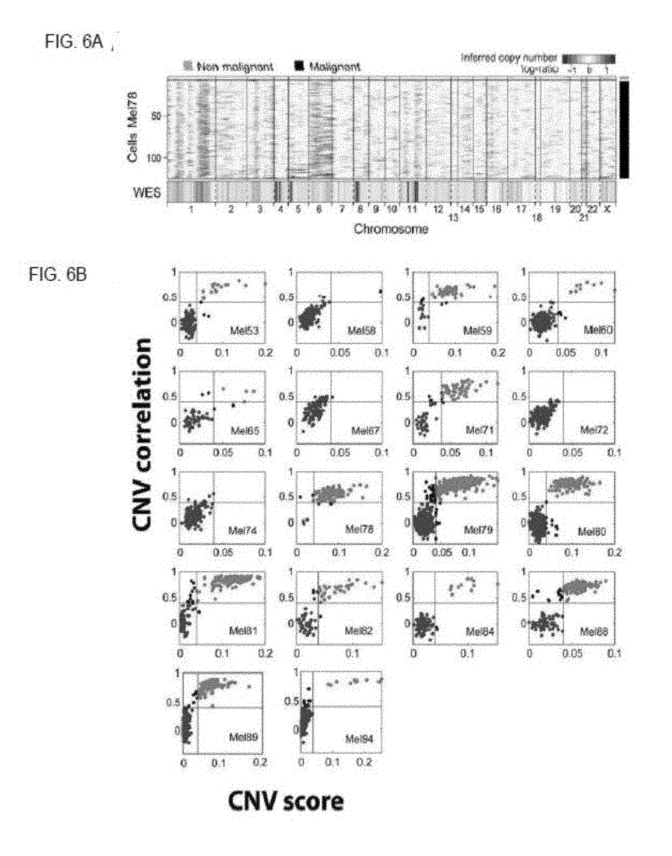

To quantitatively evaluate how likely each cell is to be a malignant or non-malignant cell Applicants summarized the CNV pattern of each cell by two values: (1) overall CNV signal, defined as the sum of squares of the CNVf estimates across all windows; (2) the correlation of each cells' CNVf vector with the average CNVf vector of the top 10% of cells from the same tumor with respect to CNV signal (i.e., the most confidently-assigned malignant cells). These two values were used to classify cells as malignant, non-malignant, and intermediates that were excluded from further analysis, as shown in FIG. 6B.

T-SNE Analysis and Cell Type Classification

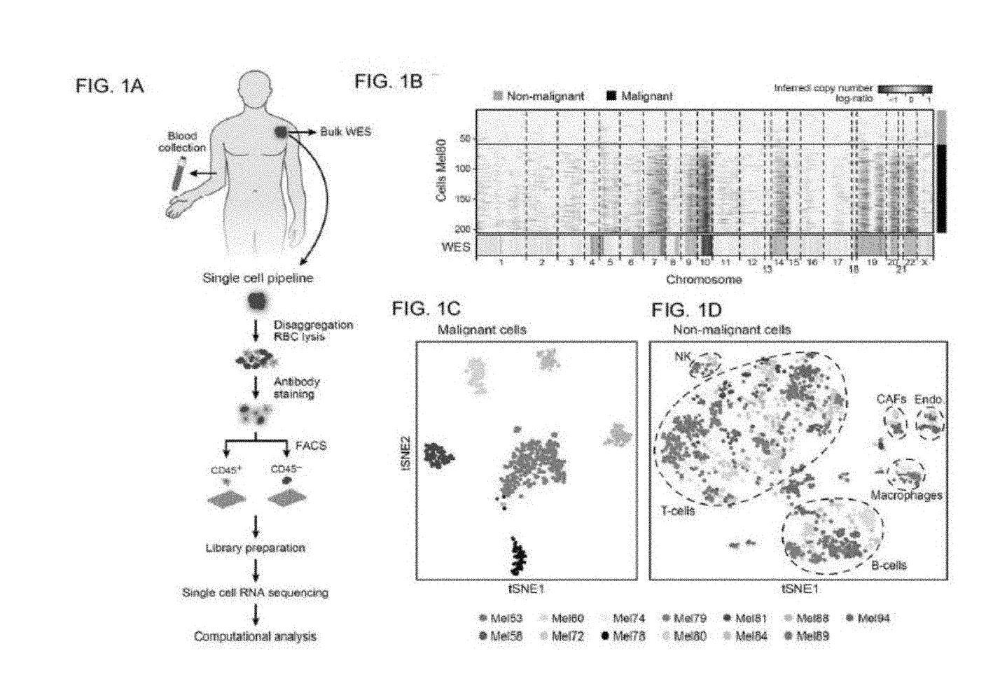

A Matlab implementation of the tSNE method was downloaded from lvdmaaten.github.io/tsne/ and applied with dim=15 to the relative expression data of malignant and to that of non-malignant cells. Since the complexity of tSNE visualization increases with the number of tumors Applicants restricted the analysis presented in FIG. 1 to the 13 tumors with at least 100 cells, and for the malignant cell analysis Applicants further restricted the analysis to 6 tumors with >50 malignant cells. To define cell types from the non-malignant tSNE analysis Applicants used a density clustering method, DBscan (18). This process revealed six clusters for which the top preferentially expressed genes (p<0.001, permutation test) included multiple known markers of particular cell types. In this way, Applicants identified T cell, B-cell, macrophage, endothelial, CAF (cancer-associated fibroblast) and NK cell clusters, as marked in FIG. 1D (dashed ellipses). To ensure the specificity of our assignment of individual cells to each cell type cluster, while avoiding potential doublet cells (which might be composed of two cells from distinct cell types), cells with low-quality data, and cells that spuriously cluster with a certain cell type, Applicants next scored each non-malignant cell (by CNV estimates, as described above) by the average expression of the identified cell type marker genes. Cells were classified as each cell type only if they express the marker genes for that cell type much more than those for any other cell type (average relative expression, Er, of markers for one cell type higher by at least 3 than those of other cell types, which corresponds to 8-fold expression difference). A full list of the genes preferentially expressed in each cell type as well as the subset that were used as marker genes is given in Table 3.

TABLE 3
cell-type specific genes.

| T-cells | B-cells | Macrophages | Endothelial cells | CAFs | melanoma |
|---|---|---|---|---|---|
| ‘CD2’ | ‘CD19’ | ‘CD163’ | ‘PECAM1’ | ‘FAP’ | ‘MIA’ |
| ‘CD3D’ | ‘CD79A’ | ‘CD14’ | ‘VWF’ | THY1 | ‘TYR’ |
| ‘CD3E’ | ‘CD79B’ | ‘CSF1R’ | ‘CDH5’ | DCN | ‘SLC45A2’ |
| ‘CD3G’ | ‘BLK’ | ‘C1QC’ | ‘CLDN5’ | ‘COL1A1’ | ‘CDH19’ |
| ‘CD8A’ | ‘MS4A1’ | ‘VSIG4’ | ‘PLVAP’ | ‘COL1A2’ | ‘PMEL’ |
| ‘SIRPG’ | ‘BANK1’ | ‘C1QA’ | ‘ECSCR’ | ‘COL6A1’ | ‘SLC24A5’ |
| ‘TIGIT’ | ‘IGLL3P’ | ‘FCER1G’ | ‘SLCO2A1’ | ‘COL6A2’ | ‘MAGEA6’ |
| ‘GZMK’ | ‘FCRL1’ | ‘F13A1’ | ‘CCL14’ | COL6A3’ | ‘GJB1’ |
| ‘ITK’ | ‘PAX5’ | ‘TYROBP’ | ‘MMRN1’ | ‘CXCL14’ | ‘PLP1’ |
| ‘SH2D1A’ | ‘CLEC17A’ | ‘MSR1’ | ‘MYCT1’ | ‘LUM’ | ‘PRAME’ |
| ‘CD247’ | ‘CD22’ | ‘C1QB’ | ‘KDR’ | ‘COL3A1’ | ‘CAPN3’ |
| ‘PRF1’ | ‘BCL11A’ | ‘MS4A4A’ | ‘TM4SF18’ | ‘DPT’ | ‘ERBB3’ |
| ‘NKG7’ | ‘VPREB3’ | ‘FPR1’ | ‘TIE1’ | ‘ISLR’ | ‘GPM6B’ |
| ‘IL2RB’ | ‘HLA-DOB’ | ‘S100A9’ | ‘ERG’ | ‘PODN’ | ‘S100B’ |
| ‘SH2D2A’ | ‘STAP1’ | ‘IGSF6’ | ‘FABP4’ | ‘CD248’ | ‘FXYD3’ |
| ‘KLRK1’ | ‘FAM129C’ | ‘LILRB4’ | ‘SDPR’ | ‘FGF7’ | ‘PAX3’ |
| ‘ZAP70’ | ‘TLR10’ | ‘FPR3’ | ‘HYAL2’ | ‘MXRA8’ | ‘S100A1’ |
| ‘CD7’ | ‘RALGPS2’ | ‘SIGLEC1’ | ‘FLT4’ | ‘PDGFRL’ | ‘MLANA’ |
| ‘CST7’ | ‘AFF3’ | ‘LILRA1’ | ‘EGFL7’ | ‘COL14A1’ | ‘SLC26A2’ |
| ‘LAT’ | ‘POU2AF1’ | ‘LYZ’ | ‘ESAM’ | MFAP5’ | ‘GPR143’ |
| ‘PYHIN1’ | ‘CXCR5’ | ‘HK3’ | CXorf36’ | ‘MEG3’ | ‘CSPG4’ |
| ‘SLA2’ | ‘PLCG2’ | ‘SLC11A1’ | ‘TEK’ | ‘SULF1’ | ‘SOX10’ |
| ‘STAT4’ | ‘HVCN1’ | ‘CSF3R’ | ‘TSPAN18’ | ‘AOX1’ | ‘MLPH’ |
| ‘CD6’ | ‘CCR6’ | ‘CD300E’ | ‘EMCN’ | ‘SVEP1’ | ‘LOXL4’ |
| ‘CCL5’ | ‘P2RX5’ | ‘PILRA’ | ‘MMRN2’ | ‘LPAR1’ | ‘PLEKHB1’ |
| ‘CD96’ | ‘BLNK’ | ‘FCGR3A’ | ‘ELTD1’ | ‘PDGFRB’ | ‘RAB38’ |
| ‘TC2N’ | ‘KIAA0226L’ | ‘AIF1’ | ‘PDE2A’ | ‘TAGLN’ | ‘QPCT’ |
| ‘FYN’ | ‘POU2F2’ | ‘SIGLEC9’ | ‘NOS3’ | ‘IGFBP6’ | ‘BIRC7’ |
| ‘LCK’ | ‘IRF8’ | ‘FCGR1C’ | ‘ROBO4’ | ‘FBLN1’ | ‘MFI2’ |
| ‘TCF7’ | ‘FCRLA’ | ‘OLR1’ | ‘APOLD1’ | ‘CA12’ | ‘LINC00473’ |
| ‘TOX’ | ‘CD37’ | ‘TLR2’ | ‘PTPRB’ | ‘SPOCK1’ | ‘SEMA3B’ |
| ‘IL32’ | | ‘LILRB2’ | ‘RHOJ’ | ‘TPM2’ | ‘SERPINA3’ |
| ‘SPOCK2’ | | ‘C5AR1’ | ‘RAMP2’ | ‘THBS2’ | ‘PIR’ |
| ‘SKAP1’ | | ‘FCGR1A’ | ‘GPR116’ | ‘FBLN5’ | ‘MITF’ |
| ‘CD28’ | | ‘MS4A6A’ | ‘F2RL3’ | ‘TMEM119’ | ‘ST6GALNAC2’ |
| ‘CBLB’ | | ‘C3AR1’ | ‘JUP’ | ‘ADAM33’ | ‘ROPN1B’ |
| ‘APOBEC3G’ | | ‘HCK’ | ‘CCBP2’ | ‘PRRX1’ | ‘CDH1’ |
| ‘PRDM1’ | | ‘IL4I1’ | ‘GPR146’ | ‘PCOLCE’ | ‘ABCB5’ |
| | | ‘LST1’ | ‘RGS16’ | ‘IGF2’ | ‘QDPR’ |
| | | ‘LILRA5’ | ‘TSPAN7’ | ‘GFPT2’ | ‘SERPINE2’ |
| | | ‘CSTA’ | ‘RAMP3’ | ‘PDGFRA’ | ‘ATP1A1’ |
| | | ‘IFI30’ | ‘PLA2G4C’ | ‘CRISPLD2’ | ‘ST3GAL4’ |
| | | ‘CD68’ | ‘TGM2’ | ‘CPE’ | ‘CDK2’ |
| | | ‘TBXAS1’ | ‘LDB2’ | ‘F3’ | ‘ACSL3’ |
| | | ‘FCGR1B’ | ‘PRCP’ | ‘MFAP4’ | ‘NT5DC3’ |
| | | ‘LILRA6’ | ‘ID1’ | ‘C1S’ | ‘IGSF8’ |
| | | ‘CXCL16’ | ‘SMAD1’ | ‘PTGIS’ | ‘MBP’ |
| | | ‘NCF2’ | ‘AFAP1L1’ | ‘LOX’ | |
| | | ‘RAB20’ | ‘ELK3’ | ‘CYP1B1’ | |
| | | ‘MS4A7’ | ‘ANGPT2’ | ‘CLDN11’ | |
| | | ‘NLRP3’ | ‘LYVE1’ | ‘SERPINF1’ | |
| | | ‘LRRC25’ | ‘ARHGAP29’ | ‘OLFML3’ | |
| | | ‘ADAP2’ | ‘IL3RA’ | ‘COL5A2’ | |
| | | ‘SPP1’ | ‘ADCY4’ | ‘ACTA2’ | |
| | | ‘CCR1’ | ‘TFPI’ | ‘MSC’ | |
| | | ‘TNFSF13’ | ‘TNFAIP1’ | ‘VASN’ | |
| | | ‘RASSF4’ | ‘SYT15’ | ‘ABI3BP’ | |
| | | ‘SERPINA1’ | ‘DYSF’ | ‘C1R’ | |
| | | ‘MAFB’ | ‘PODXL’ | ‘ANTXR1’ | |
| | | ‘IL18’ | ‘SEMA3A’ | ‘MGST1’ | |
| | | ‘FGL2’ | ‘DOCK9’ | ‘C3’ | |
| | | ‘SIRPB1’ | ‘F8’ | ‘PALLD’ | |
| | | ‘CLEC4A’ | ‘NPDC1’ | ‘FBN1’ | |
| | | ‘MNDA’ | ‘TSPAN15’ | ‘CPXM1’ | |
| | | ‘FCGR2A’ | ‘CD34’ | ‘CYBRD1’ | |
| | | ‘CLEC7A’ | ‘THBD’ | ‘IGFBP5’ | |
| | | ‘SLAMF8’ | ‘ITGB4’ | ‘PRELP’ | |
| | | ‘SLC7A7’ | ‘RASA4’ | ‘PAPSS2’ | |
| | | ‘ITGAX’ | ‘COL4A1’ | ‘MMP2’ | |
| | | ‘BCL2A1’ | ‘ECE1’ | ‘CKAP4’ | |
| | | ‘PLAUR’ | ‘GFOD2’ | ‘CCDC80’ | |
| | | ‘SLCO2B1’ | ‘EFNA1’ | ‘ADAMTS2’ | |
| | | ‘PLBD1’ | ‘PVRL2’ | ‘TPM1’ | |
| | | ‘APOC1’ | ‘GNG11’ | ‘PCSK5’ | |
| | | ‘RNF144B’ | ‘HERC2P2’ | ‘ELN’ | |
| | | ‘SLC31A2’ | ‘MALL’ | ‘CXCL12’ | |
| | | ‘PTAFR’ | ‘HERC2P9’ | ‘OLFML2B’ | |
| | | ‘NINJ1’ | ‘PPM1F’ | ‘PLAC9’ | |
| | | ‘ITGAM’ | ‘PKP4’ | ‘RCN3’ | |
| | | ‘CPVL’ | ‘LIMS3’ | ‘LTBP2’ | |
| | | ‘PLIN2’ | ‘CD9’ | ‘NID2’ | |
| | | ‘C1orf162’ | ‘RAI14’ | ‘SCARA3’ | |
| | | ‘FTL’ | ‘ZNF521’ | ‘AMOTL2’ | |
| | | ‘LIPA’ | ‘RGL2’ | ‘TPST1’ | |
| | | ‘CD86’ | ‘HSPG2’ | ‘MIR100HG’ | |
| | | ‘GLUL’ | ‘TGFBR2’ | ‘CTGF’ | |
| | | ‘FGR’ | ‘RBP1’ | ‘RARRES2’ | |
| | | ‘GK’ | ‘FXYD6’ | ‘FHL2’ | |
| | | ‘TYMP’ | ‘MATN2’ | | |
| | | ‘GPX1’ | ‘S1PR1’ | | |
| | | ‘NPL’ | ‘PIEZO1’ | | |
| | | ‘ACSL1’ | ‘PDGFA’ | | |
| | | | ‘ADAM15’ | | |
| | | | ‘HAPLN3’ | | |
| | | | ‘APP’ | | |

For each of the six cell types the list includes selected marker genes (bolded, at top) followed by all other genes defined as cell type-specific.
Non-markers genes are ordered from most (top) to least (bottom) significant, as defined by the expression difference in the respective cell type compared to all other cell types.

Principal Component Analysis

In order to decrease the impact of inter-tumoral variability on the combined analysis of cancer cells Applicants re-centered the data within each tumor separately, such that the average of each gene was zero among cells from each tumor. The covariance matrix used for PCA was generated using an approach outlined in Shalek et al. (61) to decrease the weight of less reliable “missing” values in the data. This approach aims to address the challenge that arises due to the limited sensitivity of single-cell RNA-seq, where many genes are not detected in a particular cell despite being expressed. This is particularly pronounced for genes that are more lowly expressed, and for cells that have lower library complexity (i.e., for which relatively fewer genes are detected), and results in non-random patterns in the data, whereby cells may cluster based on their complexity and genes may cluster based on their expression levels, rather than “true” co-variation. To mitigate this effect Applicants assign weights to missing values, such that the weight of Ei,j is proportional to the expectation that gene i will be detected in cell j given the average expression of gene i and the total complexity (number of detected genes) of cell j.

Following PCA, Applicants focused on the top six components as these were the only components that both explained a significant proportion of the variance and were significantly correlated with at least one gene, where significance was determined by comparison to the top 5% (of variance explained and of top gene correlations) from 100 control PCA analyses on shuffled data. PC1 had a high correlation (R=0.46) with the number of genes detected in each cell and Applicants did not observe a more specific biological function that may be associated with it and thus Applicants infer this to be a technically-driven component which is reflecting the systematic variation in the data due to the large differences in the quality and complexity of data for different cells. Subsequent analysis was focused on understanding the biological function of the next components PC2-6, which were associated with the cell cycle (PC2 and 6), regional heterogeneity (PC3) and MITF expression program (PC4 and 5).

Cell Cycle Analysis

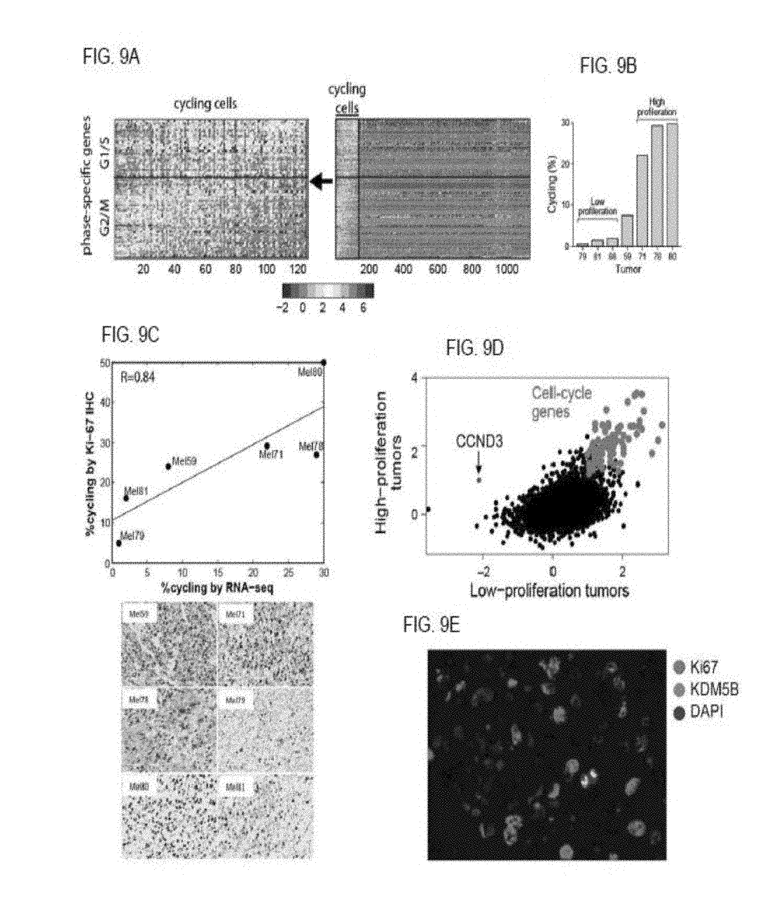

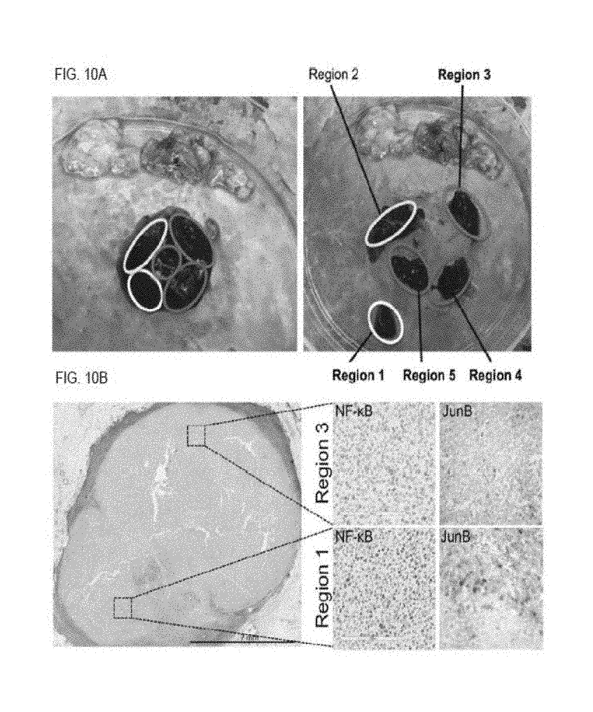

Our previous analysis of single-cell RNA-seq in human (293T) and mouse (3T3) cell lines (16), and in mouse hematopoietic stem cells (62), revealed in each case two prominent cell cycle expression programs that overlap considerably with genes that are known to function in replication and mitosis, respectively, and that have also been found to be expressed at G1/S phases and G2/M phases, respectively, in bulk samples of synchronized HeLa cells (62). Applicants thus defined a core set of 43 G1/S and 55 G2/M genes that included those genes that were detected in the corresponding expression clusters in all four datasets from the three studies described above (Table 5). Averaging the relative expression of these gene-sets revealed cells that express primarily one of those programs, or both, while the majority of the cells do not express either of those programs (FIG. 9). Applicants classified cells by the maximal expression of those two programs into non-cycling (E<1 or FDR>0.05) and cycling (E>1 and FDR<0.05) which were further divided into those with a low cell cycle signal (1<E<2), which are likely cycling but may include some false positives or arrested cells, and those with a high signal for the cell cycle (E>2) which Applicants consider as confidently cycling cells. Applicants noticed that of the 7 tumors for which Applicants have >50 malignant cells, 6 have either very low (<3%) or very high (>20%) percentage of cycling malignant cells.

Region-Specific Expression Program of Melanoma 79

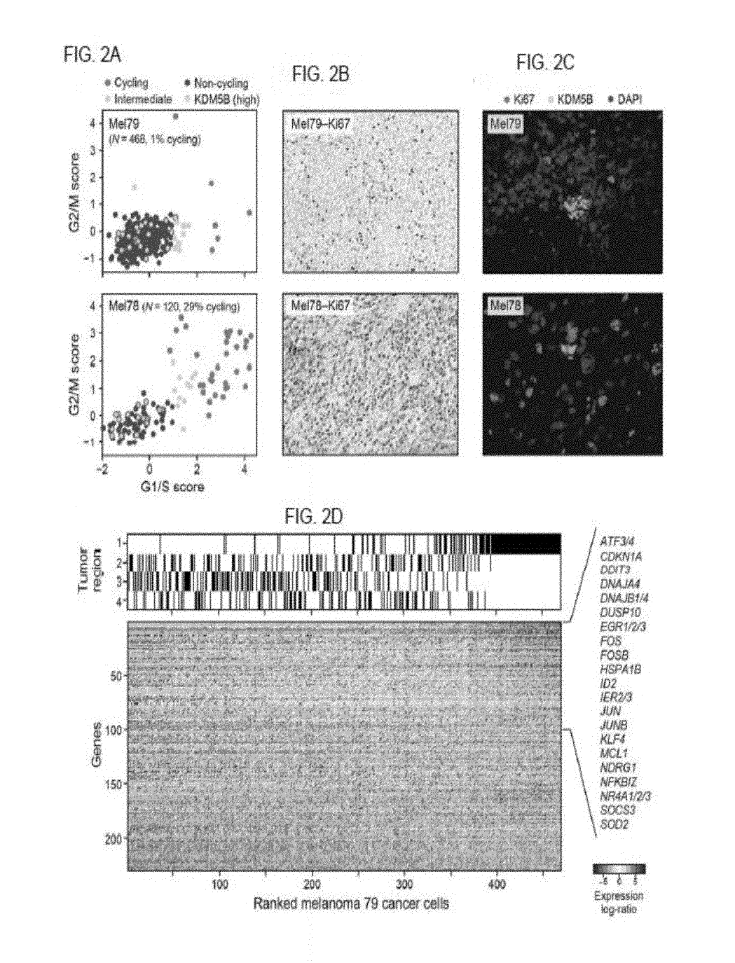

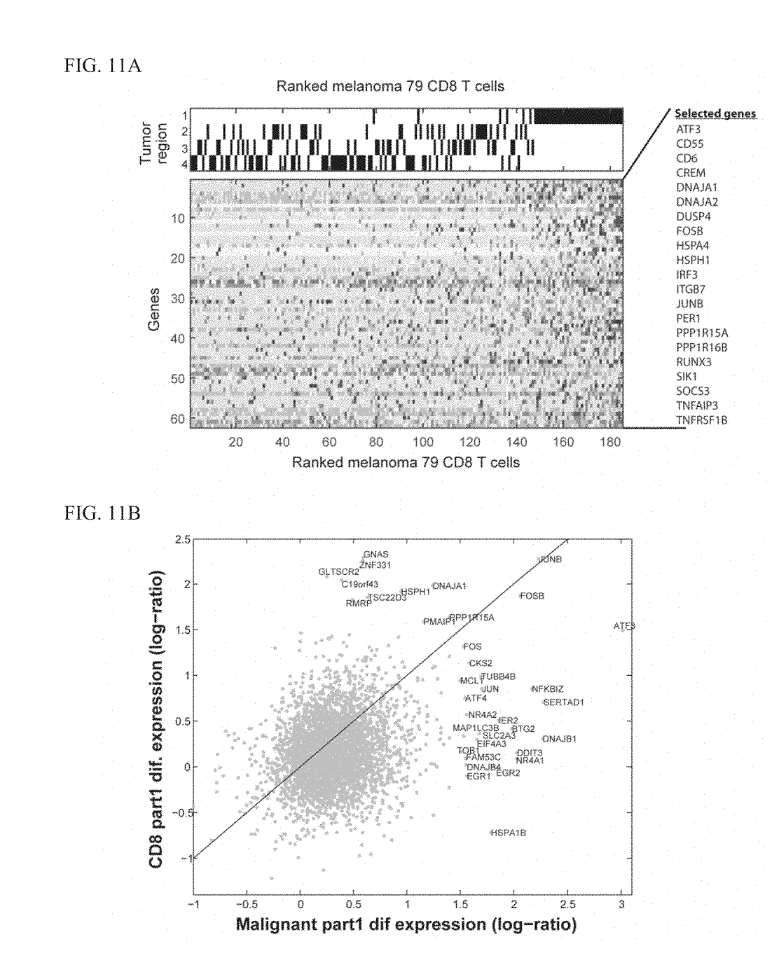

Genes with an average fold change >3 and FDR <0.05 (based both on a permutation test and a t-test with correction for multiple testing) in a comparison between either malignant (FIG. 2D) or CD8+ T (FIG. 11) cells from Region 1 and the corresponding cells from the other parts were defined as preferentially expressed in region1. Malignant or CD8+ T cells from Mel79 were then sorted by their average expression of these genes.

MITF and AXL Expression Programs and Cell Scores

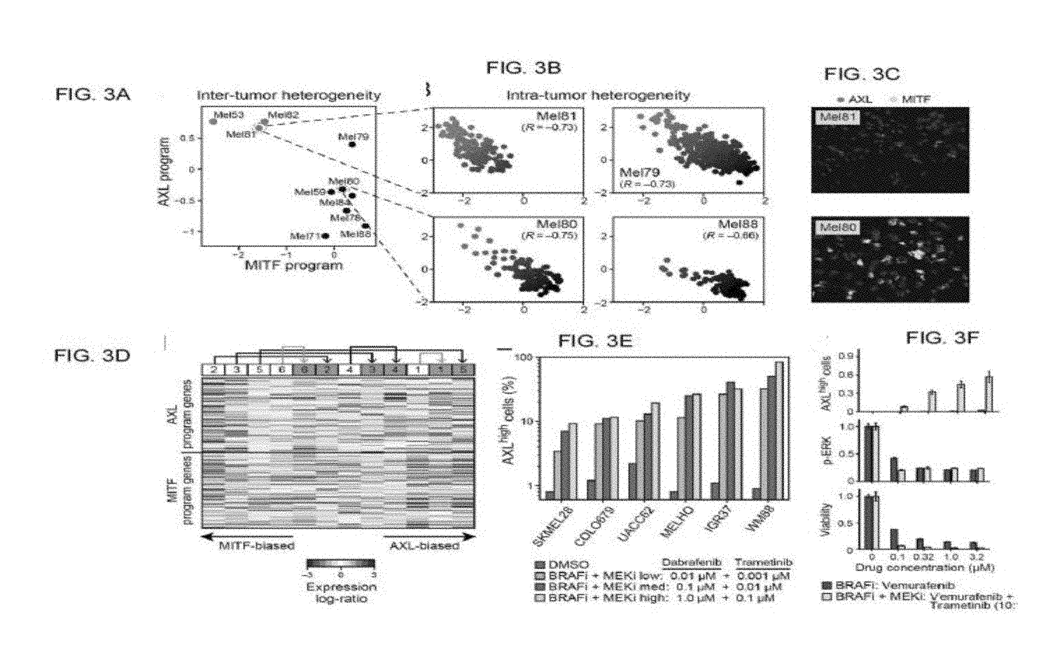

The top 100 MITF-correlated genes across the entire set of malignant cells were defined as the MITF program, and their average relative expression as the MITF-program cell score. The average expression of the top 100 genes that negatively correlate with the MITF program scores were defined as the AXL program and used to define AXL program cell score. To decrease the effect that the quality and complexity of each cell's data might have on its MITF/AXL scores Applicants defined control gene-sets and their average relative expression as control scores, for both the MITF and AXL programs. These control cell scores were subtracted from the respective MITF/AXL cell scores. The control gene-sets were defined by first binning all analyzed genes into 25 bins of aggregate expression levels and then, for each gene in the MITF/AXL gene-set, randomly selecting 100 genes from the same expression bin as that gene. In this way, a control gene-sets have a comparable distribution of expression levels to that of the MITF/AXL gene-set and the control gene set is 100-fold larger, such that its average expression is analogous to averaging over 100 randomly-selected gene-sets of the same size as the MITF/AXL gene-set. To calculate significance of the changes in AXL and MITF programs upon relapse, Applicants defined the expression log 2-ratio between matched pre- and post-samples for all AXL and MITF program genes (FIG. 3D). Since AXL and MITF programs are inversely related, Applicants flipped the signs of the log-ratios for MITF program genes and used a t-test to examine if the average of the combined set of AXL program and (sign-flipped) MITF program genes is significantly higher than zero, which was the case for four out of six matched sample pairs (FIG. 3D, black arrows)

Cell Type-Specific Signatures and Deconvolution of Bulk Expression Profiles

For each of the five main cell types identified in FIG. 1 (T cells, B cells, macrophages, endothelial cells and CAFs), Applicants defined cell type specific genes as those: (1) with average relative expression above 3 (i.e. approximately 8-fold higher than other cells); (2) expressed by >50% of the cells in that cell type; and, (3) P<0.001 when comparing cells classified into that cell type to those in each other cell type. Pvalues were determined for each pairwise comparison of cell types by comparing the observed foldchange to that seen between 10,000 pairs of control sets. The control sets were generated such that each pair is mutually exclusive, has the same number of cells as classified to the two cell types, and each set is composed of equal number of cells from the two cell types. NK cells were not included in this analysis due to their small number and limited differences from T cells, and thus the T cell signature may also identify NK cells. Next, Applicants downloaded the melanoma TCGA RNA-seqV2 expression dataset (37) and log 2-transformed the RSEM-based gene quantifications and estimated the relative frequency of each cell type by the average log-transformed expression of the cell type specific genes defined above.

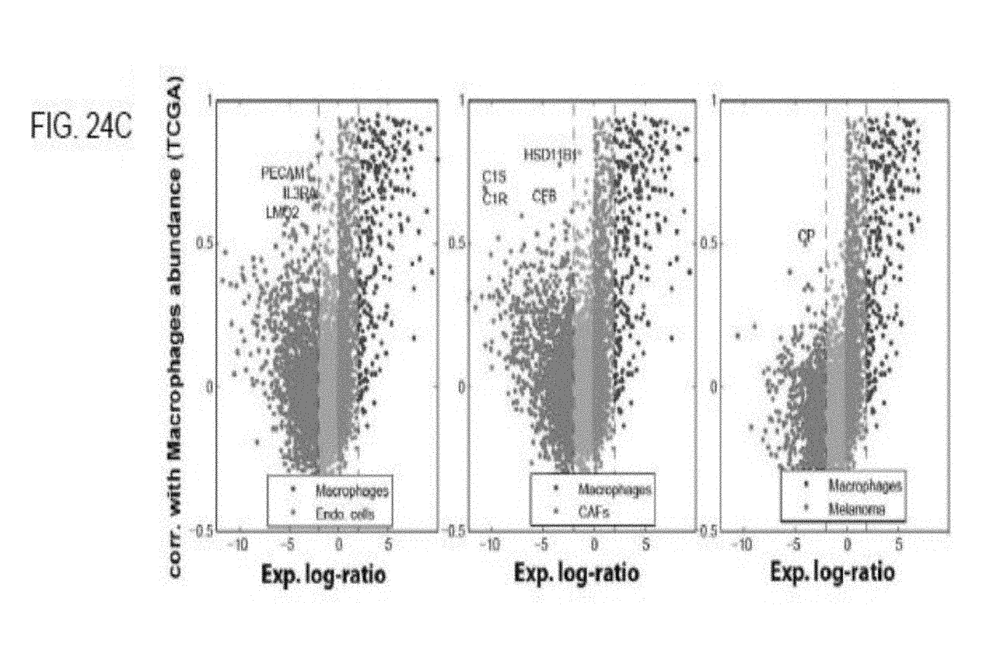

To identify genes that may mediate interactions between cell types Applicants examined the correlation between the expression of genes that are expressed primarily by one cell type, based on single cell profiles, and the relative frequency of another cell type, based on bulk TCGA profiles. Applicants focused on comparison of T cells and CAFs and identified a set of genes that although they have much higher expression in CAFs than in T cells (fold-change >4 across single cells), their expression in bulk tumors is highly correlated (R>0.5) with the estimated relative abundance of T cells (Table 15). The correlation between complement expression (the CAF signature) and T cell proportion (the T cell signature) is maintained in many cancer, and far less/non existent in normal tissues in GTEX. A similar analysis was performed for all other pairs of cell-types (FIG. 24). These are candidates for therapeutic manipulation.

TABLE 15
CAF-expressed genes that correlate with the abundance of T-cells

| CAF-expressed, | | | | |
|---|---|---|---|---|
| T/B-cell | corr. | corr. | Exp(Stroma) − | Exp(Stroma) − |
| correlated genes | With T | With B | Exp(T) | Exp(B) |
| C1S | 0.6427 | 0.5602 | 8.5056 | 9.1346 |
| UBD | 0.8315 | 0.6448 | 7.4089 | 6.6673 |
| SERPING1 | 0.654 | 0.5038 | 7.8987 | 6.7935 |
| CCL19 | 0.6804 | 0.8174 | 7.3149 | 7.7101 |
| C3 | 0.6218 | 0.6592 | 7.376 | 7.9377 |
| TGM2 | 0.5066 | 0.4779 | 7.2166 | 7.4967 |
| CXCL9 | 0.8843 | 0.6474 | 6.05 | 5.0659 |
| CXCL12 | 0.6146 | 0.6264 | 6.8387 | 7.6955 |
| TMEM176A | 0.7123 | 0.6878 | 6.5212 | 6.1329 |
| TMEM176B | 0.7597 | 0.6944 | 6.3695 | 6.355 |
| STAB1 | 0.5043 | 0.5036 | 6.9587 | 7.123 |
| CCL2 | 0.5939 | 0.5702 | 6.6362 | 6.5794 |
| PLXDC2 | 0.5126 | 0.4198 | 6.4016 | 5.8247 |
| C1R | 0.5927 | 0.5121 | 6.0416 | 8.8604 |
| CLIC2 | 0.6149 | 0.5437 | 5.9547 | 5.2628 |
| ALDH2 | 0.5594 | 0.5011 | 6.0847 | 2.554 |
| IL3RA | 0.5823 | 0.6769 | 5.7522 | 5.7951 |
| FPR2 | 0.6515 | 0.4368 | 5.518 | 5.1341 |
| SERPINA1 | 0.7051 | 0.5423 | 5.2067 | 4.9607 |
| FCGR1A | 0.7911 | 0.558 | 4.9287 | 4.8433 |
| CYBB | 0.7772 | 0.6783 | 4.9267 | −0.6677 |
| FCER1G | 0.6571 | 0.5105 | 5.2772 | 5.6419 |
| CD33 | 0.6287 | 0.5308 | 5.3447 | 4.8667 |
| LMO2 | 0.6401 | 0.6525 | 5.2456 | 2.6269 |
| SLC7A7 | 0.7918 | 0.677 | 4.7193 | 1.2406 |
| CSF1R | 0.7088 | 0.6403 | 4.7985 | 4.1882 |
| C1orf54 | 0.6741 | 0.5969 | 4.8415 | 4.1724 |
| IL34 | 0.5268 | 0.5875 | 5.2006 | 4.9851 |
| C4A | 0.5342 | 0.5331 | 5.0867 | 3.6486 |
| LILRB2 | 0.8126 | 0.6318 | 4.2076 | 3.413 |
| CSF2RB | 0.8282 | 0.8371 | 4.086 | 3.2589 |
| FPR1 | 0.6026 | 0.4769 | 4.688 | 3.4311 |
| CARD9 | 0.702 | 0.607 | 4.2483 | 3.7544 |
| TNFAIP2 | 0.721 | 0.6305 | 4.1466 | 4.1593 |
| SLCO2B1 | 0.6674 | 0.6414 | 4.2601 | 4.1278 |
| PKHD1L1 | 0.5344 | 0.6724 | 4.6243 | 3.7536 |
| FCN1 | 0.6645 | 0.5696 | 4.1683 | 3.797 |
| GP1BA | 0.586 | 0.7698 | 4.4014 | 4.1461 |
| SIGLEC6 | 0.5803 | 0.7426 | 4.4152 | 1.6201 |
| CFB | 0.6177 | 0.4997 | 4.2981 | 4.5079 |
| P2RX1 | 0.7057 | 0.7816 | 4.0268 | 1.0778 |
| NR1H3 | 0.6209 | 0.5427 | 4.2767 | 3.0717 |
| GPBAR1 | 0.7153 | 0.5332 | 3.982 | 4.0663 |
| RGS18 | 0.7173 | 0.6346 | 3.9658 | 4.0236 |
| IL7 | 0.5684 | 0.5081 | 4.3512 | 2.1569 |
| IFI30 | 0.7563 | 0.6052 | 3.7497 | 0.7839 |
| CLEC12A | 0.7339 | 0.5695 | 3.7939 | 4.7004 |
| TYROBP | 0.7613 | 0.6212 | 3.704 | 3.6344 |
| HCK | 0.8049 | 0.7162 | 3.332 | 2.0961 |
| PIK3R6 | 0.7079 | 0.6681 | 3.6123 | 2.9298 |
| ADAP2 | 0.6982 | 0.5583 | 3.6361 | 1.7039 |
| CD14 | 0.65 | 0.5399 | 3.7675 | 5.0578 |
| GHRL | 0.6626 | 0.7863 | 3.6905 | 3.8084 |
| SIGLEC9 | 0.6999 | 0.5765 | 3.5768 | 4.1243 |
| TMEM37 | 0.5852 | 0.591 | 3.8859 | 3.3609 |
| LILRA1 | 0.7067 | 0.6562 | 3.501 | 2.7022 |
| DHRS9 | 0.6137 | 0.6338 | 3.7097 | 1.8531 |
| PECAM1 | 0.6303 | 0.6685 | 3.6566 | 4.0629 |
| SPI1 | 0.782 | 0.7028 | 3.1278 | 0.44 |
| IL15RA | 0.8483 | 0.7059 | 2.904 | 5.0966 |
| SLC8A1 | 0.6955 | 0.5858 | 3.336 | 3.4454 |
| RBP5 | 0.5908 | 0.7632 | 3.6363 | 4.2231 |
| FGL2 | 0.6938 | 0.58 | 3.3051 | 3.3252 |
| MNDA | 0.7768 | 0.649 | 3.041 | 1.6354 |
| VNN1 | 0.5805 | 0.5384 | 3.6243 | 3.4418 |
| FLT3 | 0.8024 | 0.8645 | 2.9555 | 2.7583 |
| SOD2 | 0.6537 | 0.483 | 3.3772 | 3.6145 |
| CXCL11 | 0.7862 | 0.5054 | 2.9284 | 1.7897 |
| CLEC10A | 0.7288 | 0.7206 | 3.075 | 1.5159 |
| KIF19 | 0.632 | 0.5924 | 3.3161 | 3.479 |
| HSD11B1 | 0.7324 | 0.6252 | 2.9007 | 5.061 |
| CXorf21 | 0.7986 | 0.7615 | 2.6654 | 1.0901 |
| KEL | 0.5108 | 0.6335 | 3.5054 | 3.4601 |
| RARRES1 | 0.5535 | 0.5304 | 3.294 | 4.2727 |
| CFP | 0.6405 | 0.7309 | 3.0086 | 5.3814 |
| TNFSF10 | 0.7397 | 0.6063 | 2.6883 | 3.7574 |
| LILRB4 | 0.8079 | 0.6724 | 2.4161 | 2.5607 |
| P2RY12 | 0.5291 | 0.4793 | 3.2508 | 0.6342 |
| RSPO3 | 0.6312 | 0.664 | 2.8586 | 3.3143 |
| FGR | 0.7674 | 0.7263 | 2.4379 | 2.5568 |
| DRAM1 | 0.6425 | 0.4365 | 2.7659 | 1.9578 |
| ANKRD22 | 0.8067 | 0.5523 | 2.2727 | 1.9429 |
| P2RY13 | 0.83 | 0.78 | 2.1731 | 1.0301 |
| CLEC4A | 0.755 | 0.6835 | 2.3837 | 0.6484 |
| HK3 | 0.7416 | 0.5854 | 2.4237 | 2.4947 |
| FBP1 | 0.652 | 0.551 | 2.6863 | 2.8232 |
| IL18BP | 0.8309 | 0.6479 | 2.0746 | 1.5386 |
| PILRA | 0.757 | 0.6081 | 2.2904 | 2.2428 |
| TFEC | 0.776 | 0.6433 | 2.1393 | 1.1232 |
| CXCL16 | 0.5645 | 0.4462 | 2.7645 | 1.5609 |
| FCGR3A | 0.7456 | 0.4996 | 2.185 | 6.9459 |
| WARS | 0.592 | 0.3048 | 2.6364 | 2.8448 |
| LAP3 | 0.646 | 0.4136 | 2.4573 | 3.1552 |
| LGMN | 0.5569 | 0.3972 | 2.6516 | 3.0199 |
| CMKLR1 | 0.7127 | 0.6338 | 2.1556 | 1.6946 |
| RBM47 | 0.6204 | 0.5302 | 2.4299 | 1.4025 |
| SLC43A2 | 0.5629 | 0.5127 | 2.5179 | 0.8269 |
| LRRC25 | 0.7206 | 0.6321 | 2.0053 | 1.3417 |
| CP | 0.573 | 0.6772 | 2.3796 | 3.0212 |
| SLC40A1 | 0.5064 | 0.5608 | 2.4482 | 5.2851 |
| MAFB | 0.5796 | 0.4531 | 2.2015 | 2.6236 |
| CD163 | 0.622 | 0.4865 | 2.0074 | 0.9562 |
| SH2D3C | 0.5986 | 0.7095 | 2.0363 | 1.6083 |
| ODF3B | 0.5278 | 0.4128 | 2.1018 | 2.2454 |
| TLR2 | 0.5331 | 0.3832 | 2.0839 | 1.1407 |

The first column include the names of genes with average expression higher in CAFs than in T-cells by at least 4-fold (based on single cell data) and with a correlation of at least 0.5 with the abundance of T-cells across TCGA tumors.
The second to fifth columns include the correlation with T and B cell abumdances, and the expression difference (log-ratio) between CAF and T or B cells.
Genes are sorted by the average of the fourth and fifth columns.

T Cell Classification

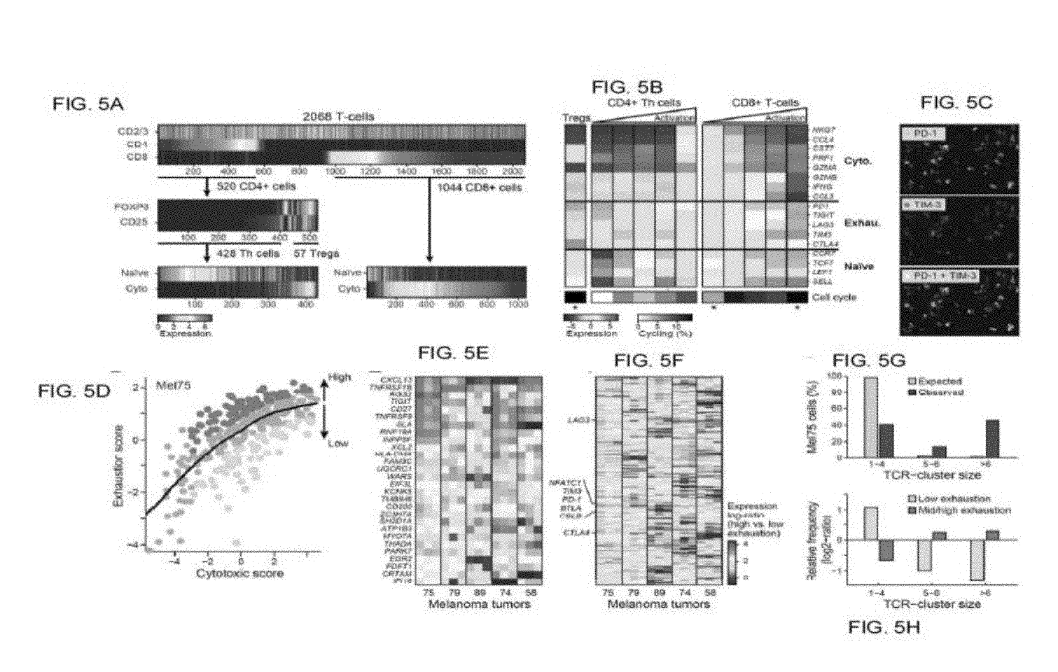

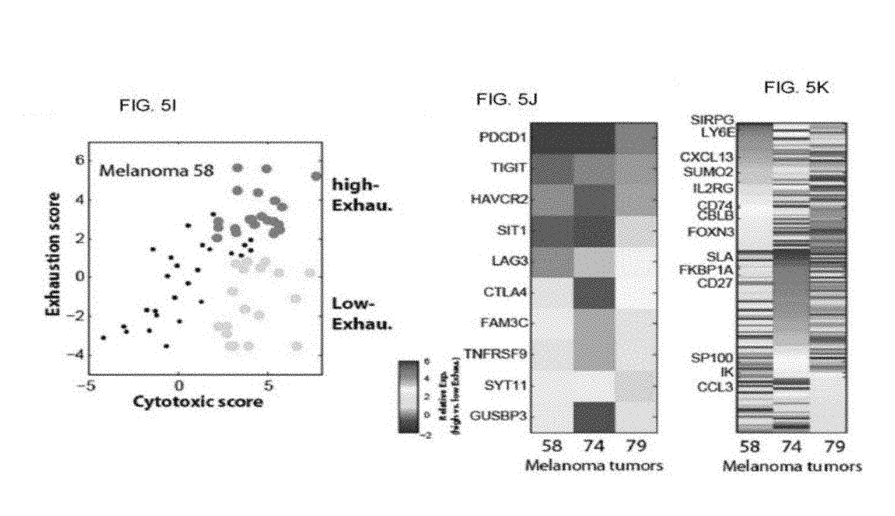

T cells were identified based on high expression of CD2 and CD3 (average of CD2, CD3D, CD3E and CD3G, E>4), and were further separated into CD4+, Tregs and CD8+ T cells based on the expression of CD4, CD25 and FOXP3, and CD8 (average of CD8A and CD8B), respectively. Applicants estimated naïve, cytotoxicity and exhaustion scores based on the average expression of the marker genes shown in FIG. 5B.

T Cell Exhaustion Analysis

Cytotoxicity and exhaustion scores were defined as the average relative expression of cytotoxic and exhaustion gene sets, respectively, minus the average relative expression of a naïve gene-set. Cytotoxic and naïve gene-sets correspond to the genes shown in FIG. 5B, while exhaustion was estimated with each of three alternative gene-sets: (1) the program identified in Mel75 (FIG. 31), and previously published gene-sets that represent (2) T cell exhaustion in melanoma (46) and (3) chronic viral infection (45). Importantly, even though the three gene-sets have limited overlap they give rise to similar exhaustion scores, and consequently exhaustion gene scores, as shown in FIG. 5E-F and Table 13, demonstrating the robustness of our analysis to the exact choice of initial exhaustion gene-sets. To estimate relative exhaustion of cells while controlling for the association between the expression of exhaustion and cytotoxicity markers. Applicants first estimated the relationship between cytotoxic and exhaustion scores using a local weighted (LOWESS) regression with a window size of 75% of the cells in each tumor (black line in FIG. 5D and FIG. 33). Due to tumor-specific patterns, this analysis was restricted to the five tumors with more than 50 CD8 T cells. Applicants then identified subsets of high exhaustion cytotoxic cells (exhaustion score −regression >0.5) and low exhaustion cells (exhaustion score −regression <−0.5), and further restricted those to cells with cytotoxic scores >−3. These thresholds were chosen to maximize the number of genes with significantly higher expression in the high-exhaustion than in the low exhaustion subsets (P<0.001 by permutation test, as described above, and fold-change >2 in at least one tumor) (provided in Table 13). Of these, genes with P<0.05 in at least three tumors were defined as consistently associated with exhaustion and are shown in FIG. 5E. Genes with P<0.05 only in one or two tumors were defined as variably associated with exhaustion and are shown in FIG. 5F. To further evaluate the significance of differential association with exhaustion across the five tumors Applicants compared the observed fold-changes between high and low exhaustion cells in each individual tumor to that seen in 10,000 control sets of high and low exhaustion cells that contain a mix of the different tumors with equal proportions (Table 13).

TABLE 13
Exhaustion program in Mel75.

| | | | |
|---|---|---|---|
| FCRL3 | HNRNPC | NAB1 | SRSF1 |
| CD27 | UBB | RAPGEF6 | GOLPH3 |
| PRKCH | CD8B | LDHA | HLA-A |
| B2M | HAVCR2 | WARS | LIMS1 |
| ITM2A | IRF8 | RASSF5 | SDF4 |
| TIGIT | LAG3 | OSBPL3 | ROCK1 |
| ID3 | ATP5B | FAM3C | EDEM1 |
| GBP2 | STAT3 | TAP1 | APLP2 |
| PDCD1 | IGFLR1 | HLA-DRB6 | ITK |
| KLRK1 | MGEA5 | FABP5 | TRIM22 |
| HSPA1A | HSPA1B | CD200 | SPRY2 |
| SRGN | COTL1 | CTLA4 | ACTG1 |
| TNFRSF9 | VCAM1 | SNX9 | HLA-DPA1 |
| TMBIM6 | HLA-DMA | ETNK1 | EWSR1 |
| TNFRSF1B | PDE7B | MALAT1 | SRSF4 |
| CADM1 | TBC1D4 | ZDHHC6 | ESYT1 |
| ACTB | SNAP47 | ARL6IP5 | LUC7L3 |
| CD8A | RGS4 | DUSP2 | ARNT |
| RGS2 | CBLB | HLA-DQB1 | GNAS |
| FAIM3 | TOX | HNRNPK | ARF6 |
| EID1 | CALM2 | DGKH | ARPC5L |
| HSPB1 | ATHL1 | LRMP | NCOA3 |
| RNF19A | SPDYE5 | H3F3B | PAPOLA |
| IFI16 | DDX5 | IDH2 | GFOD1 |
| LYST | SLA | TRAF5 | GPR174 |
| PRF1 | PTPRCAP | TBL1XR1 | DDX3X |
| STAT1 | IRF9 | ANKRD10 | CAPRIN1 |
| UBC | MATR3 | ALDOA | ARPC2 |
| CD74 | LITAF | LSP1 | PDIA6 |
| IL2RG | TPI1 | PTPN7 | SEMA4A |
| FYN | ETV1 | NSUN2 | CSDE1 |
| PTPN6 | PAM | RNF149 | PSMB9 |
| HLA-DRB1 | ARID4B | CD2 | NFATC1 |

Identification of T Cell Clones

In order to detect expanded T cell clones Applicants first mapped the transcriptome reads from each T cell to a database of TCR sequence alleles (taken from www.imgt.org/). Due to incomplete sequence coverage and sequencing errors, Applicants did not attempt to define the exact TCR sequence of each cell but instead inferred the usage of TCR alleles, including the V and J segments of the beta and the alpha chains. Applicants counted the number of reads, in each cell, which were mapped by Bowtie to each of these alleles with at most one mismatch. For each segment, a cell was defined as having a certain allele if at least two reads were mapped to that allele and no other allele was supported by half as many reads or more. Cells that did not have sufficient mapped reads to a certain segment, according to this criterion, were defined as unresolved. Applicants restricted further analysis only to the cells with at least three resolved TCR segments out of the four that were examined (V and J of alpha and beta chains). Applicants then examined all possible combinations of segments and counted, for each combination and in each tumor, the number of cells that are consistent with it and thereby define a TCR-usage cluster. Consistency was defined as having at least three identical segments and zero inconsistent segments, in order to enable cells with one unresolved segment to be classified. Cells that were consistent with multiple distinct combinations were assigned to the one with highest frequency. To evaluate the significance of clusters, Applicants performed 1,000 simulations and compared the distribution of observed cluster sizes to the combined distribution from the simulations, focusing on Mel75. In each simulation, Applicants shuffled the assignment of alleles for each segment across the Mel75 cells in which that segment was resolved, thereby preserving the structure of the data while randomizing TCR-usage clustering. Applicants separated clusters to three size ranges: 1-4 cell clusters, which were not enriched in the observed TCR usage, 5-6 cell clusters, which were enriched in the observed TCR usage but with borderline significance (FDR=0.12, defined as the fraction of cells in those clusters in the control analysis divided by the fraction of cells in the observed TCR usage), and >6 cell clusters which were highly significant (FDR=0.005). Applicants note that most Mel75 cells assigned to this last group were part of clusters with more than 10 cells, which were never observed in the simulations and are highly unlikely to occur by chance. Apart from Mel75, Applicants found a single TCR cluster of 11 cells in Mel74 (15% of cells included in TCR analysis), and no significant clusters in all other tumors.

Immunohistochemical Staining

All melanoma specimens were formalin fixed, paraffin-embedded, sectioned, and stained with hematoxylin and eosin (H&E) for histopathological evaluation at the Brigham and Women's Pathology core facility, unless otherwise specified. Immunohistochemical (IHC) studies employed 5 mm sections of formalin-fixed, paraffin-embedded tissue. All were stained on the Leica Bond III automated platform using the Leica Refine detection kit. Sections were deparaffinized and HIER was performed on the unit using EDTA for 20 minutes at 90° C. All sections were stained per routine protocols of the Brigham and Women's Pathology core facility. Additional sections were incubated for 30 min with primary antibody Ki-67 (1:250, Vector, VP-RM04) and JunB rabbit mAb (C37F9, Cell Signaling Technologies) and were then completed with the Leica Refine detection kit. The Refine detection kit encompasses the secondary antibody, the DAB chromagen (DAKO) and the Hematoxilyn counterstain. Cell counting using an ocular grid micrometer over at least five high-power fields was performed.

Tissue Immunofluorescence Staining

Dual-labeling immunofluorescence was performed to complement immunohistochemistry as a means of two-channel identification of epitopes co-expressed in similar or overlapping sub-cellular locations. Briefly, 5-mm-thick paraffin sections were incubated with primary antibodies, AXL rabbit mAb antibody (C89E7, Abcam) plus MITF mouse mAb (clone D5, ab3201, Abcam) and JAR1D1B rabbit mAb (ab56759, Abcam) plus Ki67 (ab8191, Abcam) that recognize the target epitopes at 4□C overnight and then incubated with Alexa Fluor 594-conjugated anti-mouse IgG and Alexa Fluor 488-conjugated anti-rabbit IgG (Invitrogen) at room temperature for 1 h. The sections were cover slipped with ProLong Gold anti-fade with DAPI (Invitrogen). Sections were analyzed with a BX51/BX52 microscope (Olympus America, Melville, N.Y., USA), and images were captured using the CytoVision 3.6 software (Applied Imaging, San Jose, Calif., USA). The following primary antibodies were used for staining per manufactures recommendations: mouse anti-MITF (DAKO), rabbit ant-AXL (Cell Signaling), goat anti-TIM3 (R&D Systems), rabbit ant-PD1 (Sigma Aldrich), and goat anti-PD1 (R&D Systems).

Cell Culture Experiments and AXL Flow-Cytometry

Cell lines listed in Table 11 from the Cancer Cell Encyclopedia Lines (33) were used for flowcytometry analysis of the proportion of AXL-positive cells. Based on IC50 values for vemurafenib, Applicants selected seven cell lines that were predicted to be sensitive to MAP-kinase pathway inhibition, including WM88, IGR37, MELHO, UACC62, COLO679, SKMEL28 and A375 and three cell lines predicted to be resistant, including IGR39, 294T and A2058. These ten cell lines were used for drug sensitivity testing and pre-treatment and post-treatment analysis of the AXL-positive fraction. For WM88, IGR37, MELHO, UACC62, COLO679, SKMEL28 and A375, cells were plated at a density to be at 30-50% confluent after 16 hours post seeding. A total of four drug arms were plated for each cell line using two T75 (Corning) and two T175 (Corning) culture flasks. Approximately 16-24 hours after seeding, cells were treated with DMSO or dabrafenib (D) and trametinib (T) at the following drug doses of D/T: 0.01 uM/0.001 uM, 0.1 uM/0.01 uM and 1 uM/0.1 uM (T175 reserved for higher drug concentrations). Cells were maintained in drug for a total of 5 days, at which point, cells were harvested for flow sorting. For IGR39, 294T and A2058, cells were plated at a density to be at 20-30% confluent 16 hours post seeding. Cells were treated with the DMSO or D/T at using the same doses as above and maintained in drug for a total of 10 days, at which point, cells were harvested for flow sorting. For AXL-flow sorting, cells were first washed with warm PBS, followed by an addition of 10 mM EDTA and incubated for 2 minutes at room temperature. Excess EDTA was then aspirated and cells incubated at 37° C. until cells detached from flask. Cells were resuspended in cold PBS 2% FBS and kept on ice. Cells were counted and 500,000 cells were transferred to 15 ml conical tubes (Falcon), spun down and resuspended in 100 μl of cold PBS 2% FBS alone (negative control) or antibodies using manufacturers recommendations, including 1 μg of AXL antibody (AF154, R&D Systems) or 1 μg of normal goat IgG control (Isotype control, AB-108-C, R&D Systems). Cells were incubated on ice for 1 hour, then washed twice with cold PBS 2% FBS. Cells were pelleted and resuspended in 100l PBS 2% FBS with 5 μl of Goat IgG (H+L) APC-conjugated Antibody (F0108, R&D Systems) and incubated for 30 minutes at room temperature. Cells were then washed twice with cold PBS 2% FBS, pelleted and resuspended in 500 μl of PBS 2% FBS and transferred to 5 mL flow-cytometry tubes (Falcon). 1 μl of SYTOX Blue Dead Stain (Thermo Fisher) was added to each sample and samples analyzed by flowcytometry. Data was analyzed using FACSDiva Version 6.2 using viable cells only (as determined by SYTOX Blue staining) and gates for AXL-positivity were set using the Isotype control set to <1%.

Single-Cell Immunofluorescence Staining and Analysis

For single-cell immunofluorescence (single-cell IF) studies, Applicants included the following cell lines from CCLE: WM88, MELHO, SKMEL28, COLO679, IGR39, A2058 and 294T. Cells were cultured and detached as described above, and seeded at a density of 10,000 cells per well into Costar 96-well black clear-bottom tissue culture plates (3603, Corning). Cells were treated using Hewlett-Packard (HP) D300 Digital Dispenser with vemurafenib (Selleck) alone or in combination with trametinib (Selleck) at indicated doses for 5 and 10 days. In the case of 10-day treatment, growth medium was changed after 5 days followed by immediate drug re-treatment. Cells were then fixed in 4% paraformaldehyde for 20 minutes at room temperature and washed with PBS with 0.1% Tween 20 (Sigma-Aldrich) (PBS-T), permeabilized in methanol for 10 min at room temperature, rewashed with PBS-T, and blocked in Odyssey Blocking Buffer for 1 hour at room temperature. Cells were incubated overnight at 4° C. with primary antibodies in Odyssey Blocking Buffer. The following primary antibodies with specified animal sources and catalogue numbers were used in specified dilution ratios: p-ERKT202/Y204 rabbit mAb (clone D13.14.4E, 4370, Cell Signaling Technology), 1:800, AXL goat polyclonal antibody (AF154, R&D Systems), 1:800, MITF mouse mAb (clone D5, ab3201, Abcam), 1:400, Cells were then stained with rabbit, mouse and goat secondary antibodies from Molecular Probes (Invitrogen) labeled with Alexa Fluor 647 (A31573), Alexa Fluor 488 (A21202), and Alexa Fluor 568 (A1 1057). Cells were washed once in PBS-T, once in PBS and were then incubated in 250 ng/ml Hoechst 33342 and 1:800 Whole Cell Stain (blue; Thermo Scientific) solution for 20 min. Cells were washed twice with PBS and imaged with a 10× objective on a PerkinElmer Operetta High Content Imaging System. 9-11 sites were imaged in each well. Image segmentation, analysis and signal intensity quantitation were performed using Acapella software (Perkin Elmer). Population-average and single-cell data were analyzed using MATLAB 2014b software. Single-cell density scatter plots were generated using signal intensities for individual cells.

CAF-Melanoma Co-Cultures from Melanoma 80

Solid tumor sample was removed from the transport media (Day 1: date of procurement) and minced mechanically in DMEM culture media (Thermo Scientific), 10% FCS (Gemini Bioproducts), 1% pen/strep (Life Technologies) on 10 cm culture plates (Corning Inc.) and left overnight in standard culture condition (37C, humidified atmosphere, 5% CO2). The liquid media in which the procured tissue was originally placed was spun down (1500 rpm) to isolate the detached cells in solution and the pelleted cells were resuspended in fresh culture media and propagated in culture flasks (Corning Inc.) (fraction 1). The minced tumor samples were removed from the 10 cm culture dishes on Day 2 and mechanically forced through 100 uM nylon mesh filters (Fisher Scientific) using syringe plungers and washed through with fresh culture media. The cells and tissue clumps were spun down in 50 ml conical tubes (BD Falcon), resuspended in fresh culture media, and propagated in culture flasks (fraction 2). The 10 cm culture dishes in which the samples had been minced and placed overnight were washed replaced with fresh culture media so that the attached cells could be propagated (fraction 3). Cells were propagated by changing culture media every 3-4 days and passaging cells in 1:3 to 1:6 ratio using 0.05% trypsin (Thermo Scientific) when the plates became 50-80% confluent.

Tissue Microarray Staining. Image Acquisition and Analysis

Applicants purchased two individual melanoma tissue microarrays (TMAs), including ME208 (US Biomax) and CC38-01-003 (Cybrdi). These contained a total of 308 core biopsies, including a total of 180 primary melanomas, 90 metastatic lesions, 18 melanomas with adjacent healthy skin and 20 healthy skin controls. Each TMA was double-stained with conjugated complement 3-FITC antibody (F0201. DAKO) and CD8-TRITC (ab17147, Abcam) per manufacturers recommendations. Image acquisition was performed on the RareCyte CyteFinder high-throughput imaging platform (63). For each TMAslide, the 3-channel (DAPI/FITC/TRITC) 10× images were captured and stored as Bio-format stacks. The image stacks were background-subtracted with rolling ball method and stitched into single image montage of each channel using ImageJ. For the quantification of CD8/C3 positive area and signal intensity, the gray-scale images were converted into binary images with the Otsu thresholding method (64, 65). Each tissue spot was segmented manually and DAPI. C3 and CD8-positive areas and intensities were calculated using ImageJ (NIH, MD). In order to control for sample quality, core biopsies with a DAPI staining less than 10% of total area were excluded from the correlation analysis. The raw numerical data were then processed and Pearson's correlation coefficients were calculated between C3/CD8 area fraction and intensity using MATLAB 2014b software (MathWorks, MA).

### Example 2

Profiles of Individual Cells from Patient-Derived Melanoma Tumors

Applicants measured single-cell RNA-seq profiles from 4.645 malignant, immune and stromal cells isolated from 19 freshly procured melanoma tumors that span a range of clinical and therapeutic backgrounds (Table 1). These included ten metastases to lymphoid tissues (nine to lymph nodes and one to the spleen), eight to distant sites (five to sub-cutaneous/intramuscular tissue and three to the gastrointestinal tract) and one primary acral melanoma Genotypic information was available for 17 of 19 tumors, of which four had activating mutations in BRAF and five in NRAS oncogenes; eight patients were BRAF/NRAS wild-type (Table 1).

TABLE 1
Characteristics of patients and samples included in this study Sample ID

| | | Mutation | Pre-operative | Site of | Post-op. | Alive/ |
|---|---|---|---|---|---|---|
| Sample ID | Age/sex | status | treatment | resection | treatment | deceased |
| Melanoma_53 | 77/F | Wild-type | None | Subcutaneous | None | Alive |
| | | | | back lesion | | |
| Melanoma_58 | 67/F | Wild-type | Ipilimumab | Subcutaneous | None | Alive |
| | | | | leg lesion | | |
| Melanoma_59 | 80/M | Wild-type | None | Femoral lymph | Nivolumab. | Deceased |
| | | | | node | | |
| Melanoma_60 | 69/M | BRAF | Trametinib, | Spleen | None | Alive |
| | | V600K | ipilimumab | | | |
| Melanoma_65 | 65/M | BRAF | None | Paraspinal | Neovax | Alive |
| | | V600E | | intramuscular | | |
| Melanoma_67 | 58/M | BRAF | None | Axillary lymph | None | Alive |
| | | V600E | | node | | |
| Melanoma_71 | 79/M | NRAS | None | Transverse | None | Alive |
| | | Q61L | | colon | | |
| Melanoma_72 | 57/F | NRAS | IL-2, nivolumab, | External iliac | None | Alive |
| | | Q61R | ipilimumab + anti- | lymph node | | |
| | | | KIR-Ab | | | |
| Melanoma_74 | 63/M | n/a | Nivolumab | Terminal Ileum | None | Alive |
| Melanoma_75 | 80/M | Wild-type | Ipilimumab + | Subcutaneous | Nivolumab | Alive |
| | | | nivolumab, WDVAX | leg lesion | | |
| Melanoma_78 | 73/M | NRAS | WDVAX, | Small bowel | None | Deceased |
| | | Q61L | ipilimumab + | | | |
| | | | nivolumab | | | |
| Melanoma_79 | 74/M | Wild-type | None | Axillary lymph | None | Alive |
| | | | | node | | |
| Melanoma_80 | 86/F | NRAS | None | Axillary lymph | None | Alive |
| | | Q61L | | node | | |
| Melanoma_81 | 43/F | BRAF | None | Axillary lymph | None | Alive |
| | | V600E | | node | | |
| Melanoma_82 | 81/M | Wild-type | None | Axillary lymph | None | Alive |
| | | | | node | | |
| Melanoma_84 | 67/M | Wild-type | None | Acral primary | None | Alive |
| Melanoma_88 | 54/F | NRAS | Tremelimumab + | Cutanoues met | None | Alive |
| | | Q61L | MEDI3617 | | | |
| Melanoma_89 | 67/M | n/a | None | Axillary lymph | None | Alive |
| | | | | node | | |
| Melanoma_94 | 54/F | Wild-type | IFN, ipilimumab + | Iliac lymph | None | Alive |
| | | | nivolumab | node | | |

To isolate viable single cells suitable for high-quality single-cell RNA-seq, Applicants developed and implemented a rapid translational workflow (FIG. 1A) (15). Tumor tissues were processed immediately following surgical procurement, and single-cell suspensions were generated within ˜45 minutes using an experimental protocol optimized to reduce artifactual transcriptional changes introduced by disaggregation, temperature, or time (Methods). Once in suspension, individual viable immune (CD45+) and non-immune (CD45−) cells (including malignant and stromal cells) were recovered by FACS. Next, cDNA was prepared from the individual cells, followed by library construction and massively parallel sequencing. The average number of mapped reads per cell was ˜150,000 (Methods), with a median library complexity of 4,659 genes for malignant cells and 3,438 genes for immune cells, comparable to our previous studies of only malignant cells from fresh glioblastoma tumors (15).

To limit potential artifactual transcriptional changes introduced by disaggregation, temperature or time, Applicants implemented a translational workflow to isolate viable single cells with preserved RNA quality suitable for high-quality single-cell RNA-seq (FIG. 1A). Applicants received tumor tissue for immediate processing within minutes after surgical procurement and generated a single-cell suspension within ˜40 minutes, using an optimized experimental protocol that includes mechanical and enzymatic disaggregation. Applicants stained cells for FACS with calcein-AM and CD45-FITC (and CD90-PE in some cases), to separate viable immune and non-immune cells, which included malignant and stromal cells. Notably, aside from such index-sorting, Applicants did not select of enrich for any specific sub-set of cells, opting instead for an unbiased sampling of the tumor's cellular composition. Applicants generated single-cell RNA-Seq libraries with a modified Smart-Seq2 (Picelli et al., 2013, Nature Methods 10(11):1096) protocol, as previously described, with sequencing on an Illumina NextSeq.

### Single-Cell Transcriptome Profiles Distinguish Cell States in Malignant and Non-Malignant Cells

Applicants used a multi-step approach to distinguish the different cell types within melanoma tumors based on both genetic and transcriptional states (FIG. 1B-D). First, Applicants inferred large-scale copy number variations (CNVs) from expression profiles by averaging expression over 100-gene stretches on their respective chromosomes (15) (FIG. 1B). For each tumor, this approach revealed a common pattern of aneuploidy, which Applicants validated in two tumors by bulk whole-exome sequencing (WES, FIG. 1B and FIG. 6A). Cells in which aneuploidy was inferred were classified as malignant cells (FIG. 1B and FIG. 6).

Applicants used an integrated multi-step approach to distinguish the different cell types within melanoma tumors based on both expression profiles and inferred genetic states (FIGS. 1B and C). First, Applicants inferred large-scale copy number variations (CNVs) from the expression profiles by averaging expression over 100-gene stretches on the respective chromosomes. For each tumor, this approach revealed a common pattern of aneuploidy, which Applicants validated in two tumors by bulk whole-exome sequencing (WES, FIG. 1B). Cells with CNVs were classified as malignant cells, while cells that lack these common CNVs were defined as non-malignant cells (FIG. 1B, FIG. 6).

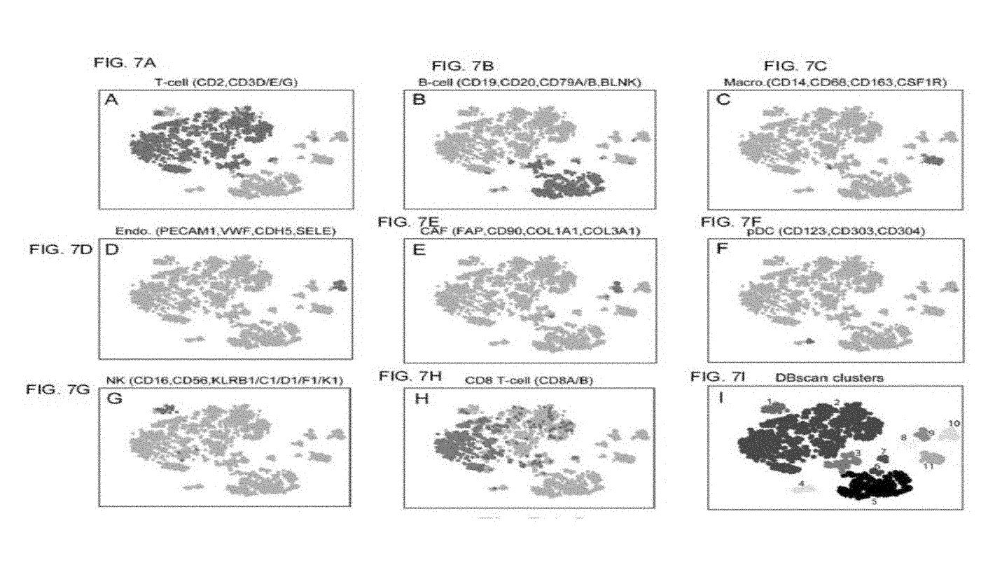

Second, Applicants grouped the cells based on their expression profiles (FIG. 1C-D, FIG. 7). Here, Applicants used non-linear dimensionality reduction (t-Distributed Stochastic Neighbor Embedding (t-SNE)) (17), followed by density clustering (18). Generally, cells designated as malignant by CNV analysis formed a separate cluster for each tumor (FIG. 1C), suggesting a high degree of inter-tumor heterogeneity. In contrast, the non-malignant cells clustered by cell type (FIG. 1D and FIG. 7), independent of their tumor of origin and metastatic site (FIG. 8). Clusters of non-malignant cells were annotated as T cells, B cells, macrophages, endothelial cells, cancer-associated fibroblasts (CAFs) and NK cells based on preferentially or uniquely expressed marker genes (FIG. 1D, FIG. 7, Table 2 and 3).

TABLE 2
Number of cells classified to each cell type from each tumor

| | T- | B- | | Endothelial | | NK | | | |
|---|---|---|---|---|---|---|---|---|---|
| | cells | cells | Macrophages | cells | CAFs | cells | Melanoma | unclassified | Total |
| All | 2068 | 515 | 126 | 65 | 61 | 52 | 1246 | 511 | 4645 |
| tumors | | | | | | | | | |
| Mel53 | 72 | 0 | 12 | 11 | 4 | 10 | 16 | 18 | 143 |
| Mel58 | 118 | 2 | 2 | 0 | 0 | 4 | 0 | 16 | 142 |
| Mel59 | 0 | 0 | 1 | 0 | 7 | 0 | 54 | 8 | 70 |
| Mel60 | 82 | 96 | 4 | 0 | 0 | 10 | 9 | 25 | 226 |
| Mel65 | 43 | 5 | 1 | 0 | 0 | 0 | 4 | 10 | 63 |
| Mel67 | 65 | 19 | 0 | 0 | 0 | 1 | 0 | 10 | 95 |
| Mel71 | 23 | 0 | 2 | 0 | 0 | 0 | 54 | 10 | 89 |
| Mel72 | 117 | 35 | 0 | 0 | 0 | 1 | 0 | 28 | 181 |
| Mel74 | 118 | 13 | 5 | 0 | 0 | 1 | 0 | 10 | 147 |
| Mel75 | 343 | 0 | 1 | 0 | 0 | 0 | 0 | 0 | 344 |
| Mel78 | 0 | 1 | 0 | 0 | 1 | 0 | 120 | 8 | 130 |
| Mel79 | 304 | 79 | 0 | 2 | 1 | 1 | 468 | 41 | 896 |
| Mel80 | 212 | 49 | 0 | 29 | 23 | 4 | 125 | 38 | 480 |
| Mel81 | 44 | 3 | 0 | 2 | 0 | 0 | 133 | 23 | 205 |
| Mel82 | 24 | 1 | 4 | 0 | 6 | 2 | 32 | 15 | 84 |
| Mel84 | 61 | 25 | 25 | 1 | 1 | 7 | 11 | 28 | 159 |
| Mel88 | 112 | 16 | 41 | 0 | 2 | 9 | 112 | 59 | 351 |
| Mel89 | 201 | 106 | 26 | 1 | 0 | 1 | 98 | 42 | 475 |
| Mel94 | 129 | 65 | 2 | 19 | 16 | 1 | 10 | 122 | 364 |

Second, Applicants used non-linear dimensionality reduction (t-Distributed Stochastic Neighbor Embedding (t-SNE)) followed by density clustering to group cells based on their expression profiles (FIG. 1C [add different shapes for tumor/non-tumor cells in the TSNE plot]). Generally, cells predicted as malignant by CNV analysis also formed a separate cluster for each tumor, indicating a high degree of inter-tumor heterogeneity in malignant cells. In contrast, cells predicted as non-malignant clustered by cell type and independently of their tumor-of-origin. Clusters of non-tumor cell were annotated as T cells, B cells, macrophages, endothelial cells and cancer-associated fibroblasts (TAFs) based on preferentially or uniquely expressed marker genes (FIG. 1C). Notably, each of the non-malignant cell clusters contained cells from multiple distinct tumors, suggesting relatively homogenous expression programs of non-malignant, melanoma-associated cells.

Analysis of Malignant Cells Reveals Heterogeneity in Cell Cycle and Spatial Organization

Applicants next used unbiased analyses of the individual malignant cells to identify biologically relevant melanoma cell states. After controlling for inter-tumor differences (Methods), Applicants examined the six top components from a principal component analysis (PCA; Table 4). The first component correlated highly with the number of genes detected per cell, and thus likely reflects technical aspects, while the other five significant principal components highlighted biological variability.

TABLE 4
PCA table including the top 50 correlated genes and the top MsigDB enrichments of those genes for the first five PCs.

| PC1 | PC2 | PC3 | PC4 | PC5 |
|---|---|---|---|---|
| PPIA | PKMYT1 | PSAP | PLP1 | PLP1 |
| EEF1A1 | CDK1 | SERPINA3 | CAPN3 | CANX |
| CFL1 | ASF1B | CSPG4 | CDH1 | ACSL3 |
| MRPL12 | TK1 | LGALS3BP | ERBB3 | DDX5 |
| ACTG1 | CDC45 | NEAT1 | S100B | TYR |
| PSMA2 | NUSAP1 | NUCB1 | RPLP1 | QPCT |
| PSMA6 | TOP2A | LAMB2 | PIR | MITF |
| ATP5G3 | BUB1 | HLA-A | STK32A | PSAP |
| ENO1 | AURKB | CTSD | TYR | CENPF |
| LDHA | CDC6 | PLXNB2 | MLANA | ETV5 |
| C1QBP | TPX2 | NBR1 | PMEL | RELL1 |
| PGAM1 | CENPF | SRRM2 | SLC24A5 | ERBB3 |
| RPLP0 | PBK | A2M | MYO10 | PTPLAD1 |
| HSPA8 | RRM2 | FLNA | HMCN1 | BIRC5 |
| SLC25A5 | CENPM | MTRNR2L6 | MITF | LOXL4 |
| RAN | BIRC5 | HSPG2 | GYG2 | CALU |
| APRT | ZWINT | AHNAK | MBP | TMEM30A |
| TOMM5 | FANCI | DDX5 | ANKS1A | TOP2A |
| PPP1CA | UBE2T | GAA | DCT | PTTG1IP |
| MDH1 | TYMS | PYGB | CRYL1 | SORT1 |
| EIF4A1 | MAD2L1 | LMNA | SEMA6A | SPSF6 |
| NHP2 | UBE2C | GRN | SLC45A2 | PBK |
| CDK4 | MLF1IP | MTRNR2L8 | TSPAN7 | AP1S2 |
| PHB | KIF2C | CD276 | GPR143 | SLC12A2 |
| RPSA | CDC20 | LTBP3 | PTPRZ1 | BUB1 |
| ATP5A1 | RFC3 | FOSB | IGSF11 | HSPA5 |
| NDUFAB1 | MCM4 | FOS | RPS18 | SDCBP |
| PSMD8 | GINS2 | SLC35F5 | RPL15 | MATN2 |
| SLC25A3 | CDKN3 | CDH19 | EXTL1 | FANCI |
| AP2S1 | KIAA0101 | C4A | CHL1 | CNP |
| DCTPP1 | CCNB2 | SLC38A2 | ABCB5 | SCARB2 |
| EIF5A | CDCA7 | PC | AHCYL2 | LAMP2 |
| ACTB | TROAP | MTRNR2L10 | LONP2 | EFNA5 |
| AP1S1 | CCNB1 | LGMN | RPL19 | TMBIM6 |
| COX7A2L | RACGAP1 | CD46 | SGCD | PDIA6 |
| HNRNPF | CENPW | MTRNR2L2 | UBL3 | SLC26A2 |
| PSMB3 | NCAPG2 | CRELD1 | VAT1 | GPNMB |
| VDAC1 | MCM2 | TMEM87B | ASAH1 | CDC20 |
| MRPS34 | MCM7 | CTSB | ETV5 | CD46 |
| LDHB | MTRNR2L2 | LRP1 | CYP27A1 | ELOVL2 |
| TUBB | ORC6 | ZNF460 | COMT | SFRP1 |
| MDH2 | MCM5 | UBA1 | RBMS3 | ITGB1 |
| NDUFB10 | TRIP13 | DAG1 | FCGR2C | TSPAN3 |
| TOMM22 | EZH2 | AFAP1 | RPL7 | GPM6B |
| SLC25A39 | MTRNR2L8 | PER1 | RPS12 | NUSAP1 |
| MTCH2 | HMGB2 | NFKBIZ | DOCK10 | ASAH1 |
| GOT2 | DNMT1 | P4HB | RGS20 | OSTM1 |
| PARK7 | KIF22 | CANX | GSTP1 | HNRNPH1 |
| CCT3 | KIF23 | ADAM10 | SCUBE2 | HPGD |
| STOML2 | DSN1 | PROS1 | ZFP106 | CTNNB1 |
| REACTOME_HOST_INTER- | CELL_CY- | REACTOME_REGULA- | STRUCTURAL_CONSTIT- | PROTEIN_HET- |
| ACTIONS_OF_HIV_FAC- | CLE_GO_0007049 | TION_OF_COMPLE- | UENT_OF_RIBOSOME | ERODIMERI- |
| TORS (7.8126) | (>16) | MENT_CASCADE | (5.0243) | ZATION_ACTIV- |
| REACTOME_GLUCO- | REACTOME_CELL_CY- | (5.1407) | REACTOME_NONSENSE_ME- | ITY (6.0762) |
| NEOGENESIS (6.8682) | CLE (>16) | REACTOME_INNATE_IM- | DIATED_DECAY_EN- | SPINDLE |
| KEGG_PARKIN- | REACTOME_CELL_CY- | MUNE_SYSTEM (4.0295) | HANCED_BY_THE_EX- | (4.4747) |
| SONS_DISEASE (6.6129) | CLE_MITOTIC (>16) | KEGG_ANTIGEN_PRO- | ON_JUNCTION_COMPLEX | KEGG_LYSO- |
| MITOCHONDRIAL_MEM- | REACTOME_MITO- | CESSING_AND_PRE- | (4.4431) | SOME (4.4148) |
| BRANE (6.1728) | TIC_M_M_G1_PHAS- | SENTATION (3.8092) | SYSTEM_DEVELOPMENT | MEMBRANE |
| REACTOME_HIV_IN- | ES (>16) | GLUCAN_METABOL- | (4.3937) | (4.4098) |
| FECTION (6.1457) | REACTOME_DNA_REP- | IC_PROCESS (3.8061) | REACTOME_SRP_DEPEN- | KEGG_MELANO- |
| | LICATION (>16) | REACTOME_LIPID_DI- | DENT_COTRANSLATION- | GENESIS |
| | | GESTION_MOBILIZA- | AL_PROTEIN_TARGET- | (2.8868) |
| | | TION_AND_TRANS- | ING_TO_MEMBRANE | |
| | | PORT (3.6338) | (4.3052) | |
| | | | PIGMENT_BIOSYNTHE- | |
| | | | TIC_PROCESS (4.2354) | |

significance for enriched MsigDB gene-sets is shown in parenthesis as −log10(P), where P is the p-value from a hypergeometric test without control for multiple testing.

The second component (PC2) was strongly associated with the expression of cell cycle genes (GO: “cell cycle” p<10⁻¹⁶; hypergeometric test). To characterize cycling cells more precisely, Applicants used gene signatures previously shown to denote G1/S or G2/M phases in both synchronization (19) and singlecell (16) experiments in cell lines. Cell cycle phase-specific signatures were highly expressed in a subset of malignant cells, thereby distinguishing cycling from non-cycling cells (FIG. 2A, FIG. 9A). These signatures revealed substantial variability in the fraction of cycling cells across tumors (13.5% on average, +/−13 STDV; FIG. 9B), thus allowing us to designate low-cycling tumors (1-3%, e.g. Mel79) and high-cycling ones (20-30%, e.g., Mel78) in a manner consistent with Ki67, staining (FIG. 2B, FIG. 9C).

A core set of known cell cycle genes was robustly induced (FIG. 9D, red dots; Table 10) in both low-cycling and high-cycling tumors, with one notable exception: cyclin D3, which was only induced in cycling cells in high-cycling tumors (FIG. 9D). In contrast, KDM5B (JAR1D1B) showed the strongest association with non-cycling cells (FIG. 2A, green dots), mirroring our recent findings in glioblastoma (15). KDM5B encodes a H3K4 histone demethylase previously associated with a subpopulation of slow-cycling and drug-resistant melanoma stem-like cells (20, 21) in mouse models. Immunofluorescence (IF) staining validated the presence and mutually exclusive expression of KDM5B and Ki67 in three representative cases. KDM5B-expressing cells were grouped in small clusters, consistent with prior observations in mouse and in vitro models (20) (FIG. 2C and FIG. 9E). These observations suggest that KDM5B may indeed exert a regulatory role in maintaining a slow-cycling subpopulation in human melanoma tumors. Importantly, cyclin D interacts with cyclin-dependent kinases (CDK4/6) for which small molecule inhibitors have shown promising results in combination with MEK inhibitors in NRAS-mutant melanoma. The pattern of CCND3 indicate that entry to the cell cycle is regulated differently in low-cycling and high-cycling tumors, which could conceivably affect the sensitivity of tumors to therapies that target cell cycle machinery, such as CDK4/6 inhibitors for which there are currently no predictive biomarkers.

TABLE 5
Cell cycle gene-sets.

| | |
|---|---|
| Phase-specific genes | melanoma cell |

| G1/S | G2/M | cycle genes |
|---|---|---|
| MCM5 | HMGB2 | TYMS |
| PCNA | CDK1 | TK1 |
| TYMS | NUSAP1 | UBE2T |
| FEN1 | UBE2C | CKS1B |
| MCM2 | BIRC5 | MCM5 |
| MCM4 | TPX2 | UBE2C |
| RRM1 | TOP2A | PCNA |
| UNG | NDC80 | MAD2L1 |
| GINS2 | CKS2 | ZWINT |
| MCM6 | NUF2 | MCM4 |
| CDCA7 | CKS1B | GMNN |
| DTL | MKI67 | MCM7 |
| PRIM1 | TMPO | NUSAP1 |
| UHRF1 | CENPF | FEN1 |
| MLF1IP | TACC3 | CDK1 |
| HELLS | FAM64A | BIRC5 |
| RFC2 | SMC4 | KIAA0101 |
| RPA2 | CCNB2 | PTTG1 |
| NASP | CKAP2L | CENPM |
| RAD51AP1 | CKAP2 | KPNA2 |
| GMNN | AURKB | CDC20 |
| WDR76 | BUB1 | GINS2 |
| SLBP | KIF11 | ASF1B |
| CCNE2 | ANP32E | RRM2 |
| UBR7 | TUBB4B | MLF1IP |
| POLD3 | GTSE1 | KIF22 |
| MSH2 | KIF20B | CDC45 |
| ATAD2 | HJURP | CDC6 |
| RAD51 | HJURP | FANCI |
| RRM2 | CDCA3 | HMGB2 |
| CDC45 | HN1 | TUBA1B |
| CDC6 | CDC20 | RRM1 |
| EXO1 | TTK | CDKN3 |
| TIPIN | CDC25C | WDR34 |
| DSCC1 | KIF2C | DTL |
| BLM | RANGAP1 | CCNB1 |
| CASP8AP2 | NCAPD2 | AURKB |
| USP1 | DLGAP5 | MCM2 |
| CLSPN | CDCA2 | CKS2 |
| POLA1 | CDCA8 | PBK |
| CHAF1B | ECT2 | TPX2 |
| BRIP1 | KIF23 | RPL39L |
| E2F8 | HMMR | SNRNP25 |
| | AURKA | TUBG1 |
| | PSRC1 | RNASEH2A |
| | ANLN | TOP2A |
| | LBR | DTYMK |
| | CKAP5 | RFC3 |
| | CENPE | CENPF |
| | CTCF | NUF2 |
| | NEK2 | BUB1 |
| | G2E3 | H2AFZ |
| | GAS2L3 | NUDT1 |
| | CBX5 | SMC4 |
| | CENPA | ANLN |
| | | RFC4 |
| | | RACGAP1 |
| | | KIFC1 |
| | | TUBB6 |
| | | ORC6 |
| | | CENPW |
| | | CCNA2 |
| | | EZH2 |
| | | NASP |
| | | DEK |
| | | TMPO |
| | | DSN1 |
| | | DHFR |
| | | KIF2C |
| | | TCF19 |
| | | HAT1 |
| | | VRK1 |
| | | SDF2L1 |
| | | PHF19 |
| | | SHCBP1 |
| | | SAE1 |
| | | CDCA5 |
| | | OIP5 |
| | | RANBP1 |
| | | LMNB1 |
| | | TROAP |
| | | RFC5 |
| | | DNMT1 |
| | | MSH2 |
| | | MND1 |
| | | TIMELESS |
| | | HMGB1 |
| | | ZWILCH |
| | | ASPM |
| | | ANP32E |
| | | POLA2 |
| | | FABP5 |
| | | TMEM194A |

phase-specific genes: genes associated with G1/S or G2/M by multiple studies, including HeLa synchronizatin and multiple single cell analysis.
melanoma core cycling genes: identified as being upregulated in cycling cells of both low-proiferation and low-proliferation melanoma tumors in this work.
Each gene-set is ranked from most significant (top) to least significant gene (bottom).

Two principal components (PC3 and PC6) primarily segregated different malignant cells from one treatment-naïve tumor (Mel79). In this case, Applicants analyzed 468 malignant cells from four distinct regions that were grossly apparent following surgical resection (FIG. 10A). Applicants identified 229 genes with higher expression in the malignant cells of Region 1 compared to those of other tumor regions (FIG. 2D, FDR<0.05; Table 6). A similar program was found in T cells from Region 1 (FIG. 11 and Table 6), suggesting a spatial effect that influences multiple cell types. Many of these genes encode immediate early activation transcription factors linked to inflammation, stress responses, and a melanoma oncogenic program (e.g., ATF3, FOS, FOSB, JUN, JUNB); several of these transcription factors (e.g., FOS, JUN, NR4A1/2) are also regulated by cyclic AMP/CREB signaling, which has recently been implicated as a possible MAP kinase-independent resistance module in BRAF-mutant melanomas treated with RAF/MEK inhibition (22). Other top genes differentially up-regulated in Region 1 included several involved in survival (MCL1), stress responses (EGR1/2/3, NDRG, HSPA1B), and NF-KB signaling (NFKBIZ), up-regulation of which has also been associated with resistance to RAF/MEK inhibition (23). Immunohistochemistiy confirmed the increased NF-KB and JunB levels in cells of Region 1 compared to the other regions of this tumor (FIG. 10B).

TABLE 6
Genes with significantly (FDR < 0.05, permutation test and t-test)
higher expression in part 1 than in parts 2-4 of melanoma79, sorted
by their significance from most (top) to least (bottom) significant.

| Malignant | CD8T-cells | shared | Gene | log-ratio (Mel) | log-ratio (CD8) |
|---|---|---|---|---|---|
| ATF3 | SIK1 | ATF3 | GLTSCR2 | 0.252222506 | 2.086409296 |
| FAM53C | C19orf43 | DNAJA1 | GNAS | 0.591640969 | 2.29668884 |
| EGR3 | RMRP | FOSB | ZNF331 | 0.583617152 | 2.257142919 |
| NFKBIZ | FOSB | HSPH1 | C19orf43 | 0.392958905 | 2.046862888 |
| SOCS3 | ZNF331 | JUNB | CXCR4 | −0.234720422 | 1.298185954 |
| FOSB | GNAS | PER1 | PSMB8 | 0.00798707 | 1.464984759 |
| NNMT | SOCS3 | PMAIP1 | DUSP4 | −0.002156341 | 1.375588499 |
| SERTAD1 | HSPH1 | PPP1R15A | RMRP | 0.490548014 | 1.833000677 |
| NR4A2 | SLC7A5P2 | RBM25 | TERF2IP | −0.009376162 | 1.273010866 |
| PAGE5 | KIAA1967 | SOCS3 | TSC22D3 | 0.636769013 | 1.86006528 |
| BTG2 | RGCC | VPS4A | TLN1 | 0.152717856 | 1.358647995 |
| KLF4 | GLTSCR2 | | CREM | 0.201817205 | 1.387282367 |
| DNAJB1 | TXNDC11 | | EZR | 0.267418963 | 1.407425319 |
| EGR2 | BAG3 | | TMEM2 | 0.27204415 | 1.405656163 |
| CHI3L1 | CCDC6 | | C9orf78 | 0.299673685 | 1.425507336 |
| NXT2 | EIF2AK1 | | TSPAN14 | 0.146641933 | 1.204816046 |
| CDKN1A | AKNA | | IRF3 | 0.222152342 | 1.214939509 |
| SLC2A3 | RASGEF1B | | C7orf49 | 0.459724154 | 1.451861912 |
| IER3 | UHRF1BP1L | | ACTN4 | 0.030988515 | 1.018958408 |
| NDRG1 | PPP1R16B | | HSPH1 | 0.943477917 | 1.919868893 |
| PMAIP1 | PER1 | | TSPYL2 | 0.407971639 | 1.361183455 |
| NR4A1 | ABCA2 | | SSU72 | 0.11169211 | 1.047236891 |
| MKNK2 | TMEM2 | | KIAA1967 | 0.271914486 | 1.16827486 |
| PER1 | C7orf49 | | AP1M1 | 0.439153317 | 1.321805129 |
| JUNB | TLN1 | | CD82 | 0.373425907 | 1.226507799 |
| TCN1 | JUNB | | ARPC5L | 0.261759923 | 1.086112011 |
| ERRFI1 | DNAJA1 | | CALM2 | 0.392575905 | 1.216503596 |
| NPTN | HSPA4 | | LNPEP | 0.226906333 | 1.049604835 |
| NUFIP2 | PFKFB3 | | CCT7 | 0.343368561 | 1.164020045 |
| SRSF7 | HNRNPU | | RPS2 | 0.244245073 | 1.060163373 |
| FLNB | TSC22D3 | | DCUN1D1 | 0.281186721 | 1.052819979 |
| DNAJB4 | RUNX3 | | DNAJA1 | 1.243459298 | 1.986808953 |
| MAFF | RBM25 | | TBCC | 0.270680713 | 1.013704745 |
| MCL1 | GGA2 | | CACYBP | 0.332256308 | 1.030562845 |
| PLEKHO2 | STK17A | | RPS4Y1 | 0.341835417 | 1.03610437 |
| CHST11 | PMAIP1 | | HSPA4 | 0.648299255 | 1.308682493 |
| MAP1LC3B | AP1M1 | | HDHD2 | 0.428757296 | 1.087748318 |
| SOD2 | C9orf78 | | FXYD5 | 0.539723273 | 1.174656358 |
| NR4A3 | USO1 | | PPP1R2 | 0.436903991 | 1.060838747 |
| TUBB3 | HDHD2 | | RAP1A | 0.416597548 | 1.038709705 |
| CKS2 | DNAJA2 | | ELOVL5 | 0.440147531 | 1.05558358 |
| DDIT3 | TMC8 | | HNRNPU | 0.606701127 | 1.203134881 |
| BRD2 | PSIP1 | | SHISA5 | 0.675317524 | 1.271566241 |
| IER2 | DCUN1D1 | | HCP5 | 0.506778059 | 1.100752716 |
| PLK3 | DUSP4 | | DNAJA2 | 0.582617829 | 1.166210107 |
| AHR | ATF3 | | USO1 | 0.627124484 | 1.204902878 |
| TMEM87B | SPOCK2 | | KAT7 | 0.470222105 | 1.038920309 |
| TOB2 | EZR | | EIF4H | 0.718204503 | 1.281212713 |
| EIF4A3 | TNFRSF1B | | DUSP2 | 0.465159328 | 1.025965098 |
| PCOLCE | YWHAZ | | SQSTM1 | 0.621100909 | 1.175767412 |
| SRSF3 | CD6 | | MAPRE1 | 0.619909542 | 1.159791778 |
| PPP1R15B | ITGB7 | | ATP1B3 | 0.661602658 | 1.177652739 |
| IFRD1 | RALY | | SLC7A5P2 | 0.705499587 | 1.218843372 |
| HSPA1B | PPP1R15A | | SRP9 | 0.918923062 | 1.421698009 |
| PAEP | VPS4A | | HSPA5 | 0.826024014 | 1.32473009 |
| SRSF2 | IRF3 | | JTB | 0.625007024 | 1.103564385 |
| YWHAG | CD55 | | CDKN1B | 0.57956218 | 1.055799156 |
| DDX3X | TSPAN14 | | PMAIP1 | 1.15225172 | 1.590623181 |
| TUBB4B | CREM | | RALY | 0.621965264 | 1.006144968 |
| MTHFD2 | TERF2IP | | RBM25 | 0.84546767 | 1.20544395 |
| MYO18A | TNFAIP3 | | GABARAPL2 | 0.736065071 | 1.082823722 |
| SERPINA3 | TSPYL2 | | RAB1B | 0.677618564 | 1.006438143 |
| TRA2B | | | RGS2 | 0.737751668 | 1.065700384 |
| CHRAC1 | | | CD55 | 0.69614823 | 1.011412363 |
| RBBP6 | | | PPP1R15A | 1.398393554 | 1.636271424 |
| DNAJA4 | | | DAZAP2 | 0.805682011 | 1.029351499 |
| RAB40B | | | YWHAZ | 0.88036532 | 1.088449689 |
| ALG13 | | | PER1 | 0.95717598 | 1.146285155 |
| EGR1 | | | EIF4A1 | 0.973990973 | 1.094262324 |
| RBM25 | | | VPS4A | 0.924950237 | 1.000271002 |
| PPP1R15A | | | JUNB | 2.228036981 | 2.276558898 |
| LRIF1 | | | SDF4 | 1.099456791 | 0.972452083 |
| TOB1 | | | SOCS3 | 1.239274706 | 1.087520763 |
| LDHA | | | DDX3X | 1.096796724 | 0.943467729 |
| H1F0 | | | BRD2 | 1.263815773 | 1.0985856 |
| FOS | | | FOSB | 2.060611028 | 1.878494149 |
| UPP1 | | | LDHA | 1.394207126 | 1.209591342 |
| HNRNPA3 | | | PGK1 | 1.144652884 | 0.951595812 |
| SSH1 | | | FOS | 1.53277235 | 1.318830452 |
| CEACAM1 | | | SLC38A2 | 1.040614705 | 0.77273943 |
| EFNA1 | | | FLOT2 | 1.003102526 | 0.710322909 |
| AMD1 | | | SRSF2 | 1.285810804 | 0.96158808 |
| DUSP10 | | | CCNI | 1.070713553 | 0.715893832 |
| PROS1 | | | AKIRIN1 | 1.096793774 | 0.707693066 |
| ATF4 | | | CKS2 | 1.581645741 | 1.141182216 |
| FTH1P3 | | | TCP1 | 1.113847445 | 0.638168184 |
| DHX40 | | | SRSF7 | 1.317717507 | 0.805261911 |
| ID2 | | | IFRD1 | 1.067791728 | 0.545153102 |
| CSF2RA | | | SURF4 | 1.110413256 | 0.587027483 |
| CCNL1 | | | HNRNPA1 | 1.184707116 | 0.659806945 |
| SERTAD3 | | | PLEKHO2 | 1.113486778 | 0.587438196 |
| JUN | | | CHRAC1 | 1.053477445 | 0.504222939 |
| ACSL1 | | | MCL1 | 1.501994807 | 0.950243499 |
| CCNI | | | ALDOC | 1.012393692 | 0.402809545 |
| ENO2 | | | DUSP10 | 1.00727568 | 0.390859828 |
| GTF2B | | | CIB1 | 1.195183423 | 0.568896377 |
| NEK6 | | | GTF2B | 1.046787923 | 0.405238101 |
| EIF1B | | | EIF1B | 1.193725902 | 0.551475552 |
| ETF1 | | | ENO1 | 1.110590698 | 0.440872249 |
| SRPX | | | VDAC1 | 1.017453681 | 0.343048166 |
| GOLGA5 | | | IDI1 | 1.038833197 | 0.359552913 |
| NFE2L3 | | | NEU1 | 1.184051287 | 0.486397167 |
| HSPH1 | | | TUBB4B | 1.694781409 | 0.989268362 |
| IL1RAP | | | ERP29 | 1.118556397 | 0.405331526 |
| TCP1 | | | TOB2 | 1.029853524 | 0.287804928 |
| PLK2 | | | PRDX4 | 1.047159318 | 0.293500338 |
| BACE2 | | | NEK6 | 1.071948265 | 0.317890975 |
| SDF4 | | | AMD1 | 1.279559988 | 0.521787891 |
| RCN1 | | | ATF4 | 1.543509694 | 0.757455201 |
| AKIRIN1 | | | PGAM1 | 1.187387547 | 0.357996451 |
| CITED1 | | | JUN | 1.703112224 | 0.855079899 |
| CIB1 | | | PDCD6 | 1.034992857 | 0.147728358 |
| TM4SF1 | | | ID2 | 1.316019092 | 0.425227751 |
| PELI1 | | | ACSL1 | 1.088429416 | 0.179136289 |
| FLOT2 | | | HPCAL1 | 1.127133375 | 0.191786238 |
| SLC44A3 | | | MAF1 | 1.182298314 | 0.241015831 |
| PJA2 | | | SRSF3 | 1.320409005 | 0.369260711 |
| CTSL1 | | | AHSA1 | 1.000218046 | 0.045288254 |
| NUCB1 | | | HNRNPF | 1.018726232 | 0.044997905 |
| CRELD1 | | | NR4A2 | 1.557340376 | 0.572682736 |
| MAF1 | | | ENO2 | 1.309820157 | 0.303844071 |
| NASP | | | CRELD1 | 1.082740151 | 0.075309902 |
| ARL4A | | | AKR1B1 | 1.015573187 | −0.0138164 |
| JMJD6 | | | SOD2 | 1.399769308 | 0.313967521 |
| CLIC4 | | | HSPA1A | 1.339418934 | 0.2457482 |
| SLC16A3 | | | LRIF1 | 1.002232947 | −0.106726418 |
| SLC1A5 | | | P4HA1 | 1.001445952 | −0.157545039 |
| TNFRSF21 | | | TUBA1C | 1.227893762 | 0.038967014 |
| SURF4 | | | MAP1LC3B | 1.531883103 | 0.339518494 |
| TUBA1C | | | SLC16A3 | 1.12378414 | −0.084961286 |
| VDAC1 | | | NXT2 | 1.175906742 | −0.03916186 |
| TNFRSF1A | | | SLC20A1 | 1.003434105 | −0.21252674 |
| ERP29 | | | DNAJA4 | 1.258691241 | 0.025806455 |
| GEM | | | ENTPD6 | 1.07261985 | −0.161384344 |
| AAMP | | | PLK3 | 1.278283141 | −0.004143908 |
| ALX1 | | | SLC2A3 | 1.674598643 | 0.36625382 |
| IDI1 | | | NFKBIZ | 2.167024852 | 0.85413723 |
| DNAJA1 | | | IER2 | 1.85358172 | 0.511122989 |
| NEU1 | | | TOB1 | 1.509794826 | 0.160990714 |
| HNRNPF | | | EIF4A3 | 1.655647654 | 0.299055634 |
| KLF10 | | | AAMP | 1.096238379 | −0.28094529 |
| PGAM1 | | | FAM53C | 1.556239773 | 0.087173711 |
| ENTPD6 | | | ATF3 | 3.019658275 | 1.491802942 |
| C4A | | | DNAJB4 | 1.551960965 | 0.020658815 |
| HNRNPA1 | | | BTG2 | 1.981447394 | 0.419203133 |
| TCTN1 | | | SERTAD1 | 2.276633358 | 0.712186012 |
| CCDC104 | | | CCNL1 | 1.041198985 | −0.556575632 |
| HIF1A | | | TM4SF1 | 1.398409435 | −0.231349813 |
| MANF | | | EGR1 | 1.562124421 | −0.102983977 |
| SERPINE1 | | | RCN1 | 1.246442578 | −0.525372259 |
| C15orf57 | | | EGR2 | 1.80614608 | −0.00291098 |
| PTP4A1 | | | DDIT3 | 2.029133028 | 0.153613626 |
| NAMPT | | | NR4A1 | 2.028975833 | 0.090997893 |
| TSSC1 | | | DNAJB1 | 2.266772656 | 0.306027159 |
| VPS4A | | | HSPA1B | 1.785643775 | −0.720875186 |
| ALDOC | | | | | |
| NOC2L | | | | | |
| TRIB1 | | | | | |
| ODC1 | | | | | |
| P4HA1 | | | | | |
| USP11 | | | | | |
| LTA4H | | | | | |
| HIST2H4A | | | | | |
| HIST2H4B | | | | | |
| UGDH | | | | | |
| TUBB2A | | | | | |
| IFNAR2 | | | | | |
| RAB34 | | | | | |
| DGCR2 | | | | | |
| POLDIP2 | | | | | |
| SPPL2A | | | | | |
| SPP1 | | | | | |
| ADAM9 | | | | | |
| ARPC4 | | | | | |
| SLC1A4 | | | | | |
| HPCAL1 | | | | | |
| C17orf62 | | | | | |
| FAM174A | | | | | |
| PTTG1 | | | | | |
| PLEKHB2 | | | | | |
| ATP6V1D | | | | | |
| ADM | | | | | |
| LITAF | | | | | |
| COPS4 | | | | | |
| PNRC2 | | | | | |
| HIAT1 | | | | | |
| GCSH | | | | | |
| NXF1 | | | | | |
| DDRGK1 | | | | | |
| PRDX4 | | | | | |
| KDELR2 | | | | | |
| PDCD6 | | | | | |
| ACLY | | | | | |
| YPEL5 | | | | | |
| EFTUD2 | | | | | |
| BZW2 | | | | | |
| LGMN | | | | | |
| TXNRD1 | | | | | |
| TATDN1 | | | | | |
| HMGN4 | | | | | |
| AHSA1 | | | | | |
| CLK1 | | | | | |
| AKR1B1 | | | | | |
| PPAPDC1B | | | | | |
| HMG20B | | | | | |
| SLC20A1 | | | | | |
| PFKP | | | | | |
| APOA1BP | | | | | |
| RNF185 | | | | | |
| DNAJB9 | | | | | |
| SLC25A39 | | | | | |
| BUD31 | | | | | |
| PEX10 | | | | | |
| SUMO3 | | | | | |
| LRRC41 | | | | | |
| RBMX | | | | | |
| MALSU1 | | | | | |
| ZNF32 | | | | | |
| IFI35 | | | | | |
| LYPLA2 | | | | | |
| TNFRSF12A | | | | | |
| RAP1B | | | | | |
| VAMP3 | | | | | |
| PARL | | | | | |
| ORMDL3 | | | | | |
| SFT2D2 | | | | | |
| YIPF3 | | | | | |
| SLC22A18 | | | | | |
| MAGEA12 | | | | | |

The first three columns contain significant genes from analysis of malignant cells (first column) CD8 T-cells (second column) and the genes shared by both analysis (third column).
The last three columns show differential expression values (log2-ratio between part1 and parts 2-4) for malignant cells and for CD8 T-cells, including all genes with at least 2-fold upregulation in one of the analysis, sorted by the difference in log-ratio between CD8 and malignant cell analysis (top genes are specifically upregulated in CD8 cells, while bottom genes are more specific to malignant cells)

Heterogeneity in the Abundance of a Dormant, Drug-Resistant Melanoma Subpopulation

Collectively, the above observations implied that some treatment-naïve melanoma tumors may harbor malignant cell subsets less likely to respond to targeted therapy. The transcriptional programs associated with two other principal components (PC4 and PC5) identified by our unbiased analysis directly support this notion. Both PC4 and PC5 were highly correlated with expression of MITF (microphthalmia-associated transcription factor), which encodes the master melanocyte transcriptional regulator and a melanoma lineage-survival oncogene (24). Scoring genes by their correlation to MITF across single cells, Applicants identified a “MITF-high” program consisting of several known MITF targets, including TYR, PMEL and MLANA (Table 7). A second transcriptional program, negatively correlated with the MITF program and with PC4 and PC5 (P<10-24), included AXL and NGFR (p75NTR), a marker of resistance to various targeted therapies (25, 26) and a putative melanoma cancer stem cell marker (27), respectively (Table 8). Thus, to a first approximation, these transcriptional programs resemble previously reported (23, 28-30) “MITF-high” and “MITF-low/AXL-high” (“AXL-high”) transcriptional profiles that distinguish melanoma tumors, cell lines and mice models. Notably, the “AXL-high” program has previously been linked to intrinsic resistance to RAF/MEK inhibition (23, 28, 29).

TABLES 7
Genes in the MITF program from single cell analysis.
MITF program was defined as the 100 genes with
highest correlations with the MITF gene.
genes are sorted from most (top) to least (bottom) significant.

| | |
|---|---|
| 1 | MITF |
| 2 | TYR |
| 3 | PMEL |
| 4 | PLP1 |
| 5 | GPR143 |
| 6 | MLANA |
| 7 | STX7 |
| 8 | IRF4 |
| 9 | ERBB3 |
| 10 | CDH1 |
| 11 | GPNMB |
| 12 | IGSF11 |
| 13 | SLC24A5 |
| 14 | SLC45A2 |
| 15 | RAP2B |
| 16 | ASAH1 |
| 17 | MYO10 |
| 18 | GRN |
| 19 | DOCK10 |
| 20 | ACSL3 |
| 21 | SORT1 |
| 22 | QPCT |
| 23 | S100B |
| 24 | MYC |
| 25 | LZTS1 |
| 26 | GYG2 |
| 27 | SDCBP |
| 28 | LOXL4 |
| 29 | ETV5 |
| 30 | C1orf85 |
| 31 | HMCN1 |
| 32 | OSTM1 |
| 33 | ALDH7A1 |
| 34 | FOSB |
| 35 | RAB38 |
| 36 | ELOVL2 |
| 37 | MLPH |
| 38 | PLK2 |
| 39 | CHL1 |
| 40 | RDH11 |
| 41 | LINC00473 |
| 42 | RELL1 |
| 43 | C21orf91 |
| 44 | SCAMP3 |
| 45 | SGK3 |
| 46 | ABCB5 |
| 47 | SLC7A5 |
| 48 | SIRPA |
| 49 | WDR91 |
| 50 | PIGS |
| 51 | CYP27A1 |
| 52 | TM7SF3 |
| 53 | PTPRZ1 |
| 54 | CNDP2 |
| 55 | CTSK |
| 56 | BNC2 |
| 57 | TOB1 |
| 58 | CELF2 |
| 59 | ROPN1 |
| 60 | TMEM98 |
| 61 | CTSA |
| 62 | LIMA1 |
| 63 | CD99 |
| 64 | IGSF8 |
| 65 | FDFT1 |
| 66 | CPNE3 |
| 67 | SLC35B4 |
| 68 | EIF3E |
| 69 | TNFRSF14 |
| 70 | VAT1 |
| 71 | HPS5 |
| 72 | CDK2 |
| 73 | CAPN3 |
| 74 | SUSD5 |
| 75 | ADSL |
| 76 | PIGY |
| 77 | PON2 |
| 78 | SLC19A1 |
| 79 | KLF6 |
| 80 | MAGED1 |
| 81 | ERGIC3 |
| 82 | PIR |
| 83 | SLC25A5 |
| 84 | JUN |
| 85 | ARPC1B |
| 86 | SLC19A2 |
| 87 | AKR7A2 |
| 88 | HPGD |
| 89 | TBC1D7 |
| 90 | TFAP2A |
| 91 | PTPLAD1 |
| 92 | SNCA |
| 93 | GNPTAB |
| 94 | DNAJA4 |
| 95 | APOE |
| 96 | MTMR2 |
| 97 | ATP6V1B2 |
| 98 | C16orf62 |
| 99 | EXOSC4 |
| 100 | STAM |

TABLES 8
Genes in the AXL program from single cell analysis.
AXL program was defined as the 100 genes with
the lowest correlations (most negative) with the average
expression of the MITF program genes.
genes are sorted from most (top) to least (bottom) significant.

| | |
|---|---|
| 1 | ANGPTL4 |
| 2 | FSTL3 |
| 3 | GPC1 |
| 4 | TMSB10 |
| 5 | SH3BGRL3 |
| 6 | PLAUR |
| 7 | NGFR |
| 8 | SEC14L2 |
| 9 | FOSL1 |
| 10 | SERPINE1 |
| 11 | IGFBP3 |
| 12 | TNFRSF12A |
| 13 | GBE1 |
| 14 | AXL |
| 15 | PHLDA2 |
| 16 | MAP1B |
| 17 | GEM |
| 18 | SLC22A4 |
| 19 | TYMP |
| 20 | TREM1 |
| 21 | RIN1 |
| 22 | S100A4 |
| 23 | COL6A2 |
| 24 | FAM46A |
| 25 | CITED1 |
| 26 | S100A10 |
| 27 | UCN2 |
| 28 | SPHK1 |
| 29 | TRIML2 |
| 30 | S100A6 |
| 31 | TMEM45A |
| 32 | CDKN1A |
| 33 | UBE2C |
| 34 | ERO1L |
| 35 | SLC16A6 |
| 36 | CHI3L1 |
| 37 | FNI |
| 38 | S100A16 |
| 39 | CRIP1 |
| 40 | SLC25A37 |
| 41 | LCN2 |
| 42 | ENO2 |
| 43 | PFKFB4 |
| 44 | SLC16A3 |
| 45 | DBNDD2 |
| 46 | LOXL2 |
| 47 | CFB |
| 48 | CADM1 |
| 49 | LTBP3 |
| 50 | CD109 |
| 51 | AIM2 |
| 52 | TCN1 |
| 53 | STRA6 |
| 54 | C9orf89 |
| 55 | DDR1 |
| 56 | TBC1D8 |
| 57 | METTL7B |
| 58 | GADD45A |
| 59 | UPP1 |
| 60 | SPATA13 |
| 61 | GLRX |
| 62 | PPFIBP1 |
| 63 | PMAIP1 |
| 64 | COL6A1 |
| 65 | JMJD6 |
| 66 | CIB1 |
| 67 | HPCAL1 |
| 68 | MT2A |
| 69 | ZCCHC6 |
| 70 | IL8 |
| 71 | TRIM47 |
| 72 | SESN2 |
| 73 | PVRL2 |
| 74 | DRAP1 |
| 75 | MTHFD2 |
| 76 | SDC4 |
| 77 | NNMT |
| 78 | PPL |
| 79 | TIMP1 |
| 80 | RHOC |
| 81 | GNB2 |
| 82 | PDXK |
| 83 | CTNNA1 |
| 84 | CD52 |
| 85 | SLC2A1 |
| 86 | BACH1 |
| 87 | ARHGEF2 |
| 88 | UBE2J1 |
| 89 | CD82 |
| 90 | ZYX |
| 91 | P4HA2 |
| 92 | PEA15 |
| 93 | GLRX2 |
| 94 | HAPLN3 |
| 95 | RAB36 |
| 96 | SOD2 |
| 97 | ESYT2 |
| 98 | IL18BP |
| 99 | FGFRL1 |
| 100 | PLEC |

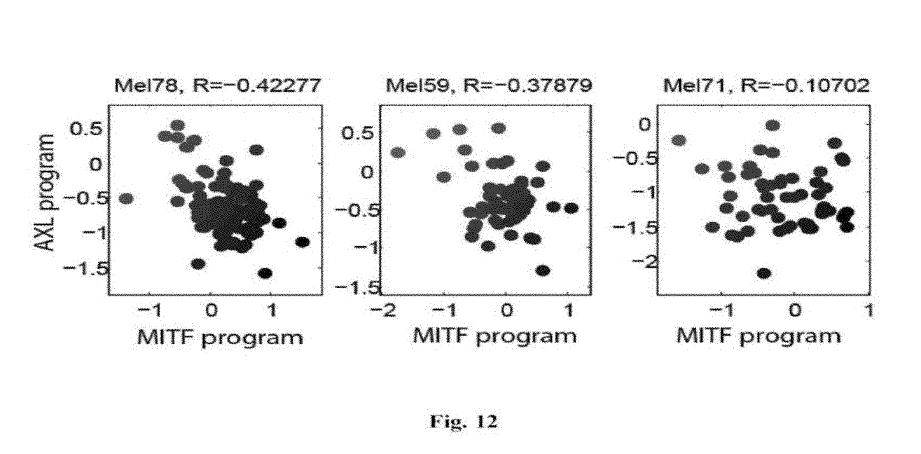

While each melanoma could be classified as “MITF-high” or “AXL-high” at the bulk tumor level (FIG. 3A), at the single cell level every tumor contained malignant cells corresponding to both transcriptional states. Using single-cell RNA-seq to examine each cell's expression of the MITF and AXL gene sets, Applicants observed that MITF-high tumors, including treatment-naïve melanomas, harbored a subpopulation of AXL-high melanoma cells that was undetectable through bulk analysis, and vice versa (FIG. 3B). The malignant cells thus spanned the continuum between AXL-high and MITF-high states in both (FIG. 3B and FIG. 12). Applicants further validated the mutually exclusive expression of the MITF-high and AXLhigh programs in cells from the same bulk tumors by immunofluorescence (FIG. 3C and FIG. 15).

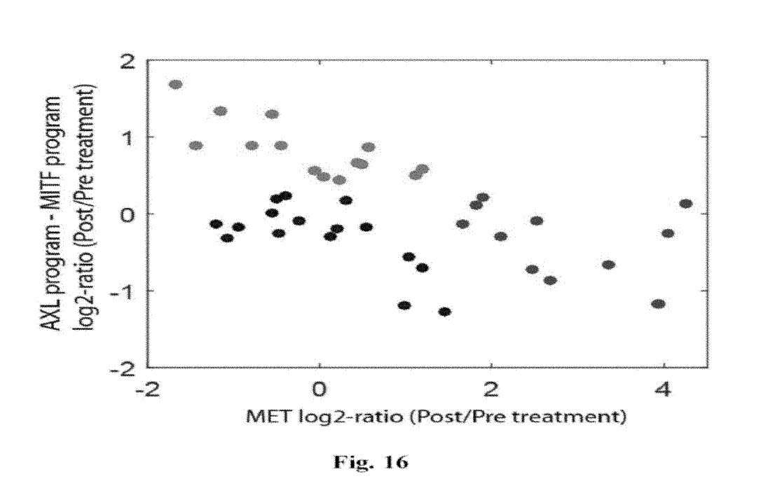

Since malignant cells with AXL-high and MITF-high transcriptional states co-exist in melanoma, Applicants hypothesized that treatment with RAF/MEK inhibitors would increase the prevalence of AXL-high cells following the development of drug resistance. To test this. Applicants analyzed RNA-seq data from a recently published cohort (13) of six paired BRAF^{V600E} melanoma biopsies taken before treatment and after resistance to single-agent RAF inhibition (vemurafenib; n=1) or combined RAF/MEK inhibition (dabrafenib and trametinib; n=5), respectively (Table 10). Applicants ranked the 12 transcriptomes based on their relative expression of all genes in the AXL-high program compared to those in the MITF-high program. In each pair, Applicants observed a shift towards the AXL-high program in the drug resistant sample, consistent with our hypothesis that AXL-high tumor cells underwent positive selection in the setting of RAF/MEK inhibition (FIG. 3D; P<0.05 for same effect in six out of six paired samples, binomial test; P<0.05 for four of six individual paired-sample comparisons shown by black arrows, Methods). RNA-seq data from an independent cohort (31) also showed that a subset of drug resistant samples exhibited increased expression of the AXL program (FIG. 16). Other genes previously implicated in resistance to RAF/MEK inhibition were also increased in a subset of the drug-resistant samples. PDGFRB (32) was upregulated in a similar subset as the AXL program, while MET (31) was upregulated in a mutually exclusive subset (FIG. 16), suggesting that AXL and MET may reflect distinct mechanisms for drug resistance.

TABLE 10
Sample information on pre-treatment and post-relapse samples (6)

| | | Best response (in % by | PFS |
|---|---|---|---|
| Patient ID | Treatment | RECIST criteria) | (months) |
| 1 | Dabrafenib/Trametinib | −100 (CR) | 18 |
| 2 | Dabrafenib/Trametinib | −20 (SD) | 10 |
| 3 | Vemurafenib | −51 (PR) | 5 |
| 4 | Dabrafenib/Trametinib | −42 (PR) | 3 |
| 5 | Dabrafenib/Trametinib | −53 (PR) | 2 |
| 6 | Dabrafenib/Trametinib | −23 (SD) | 2 |

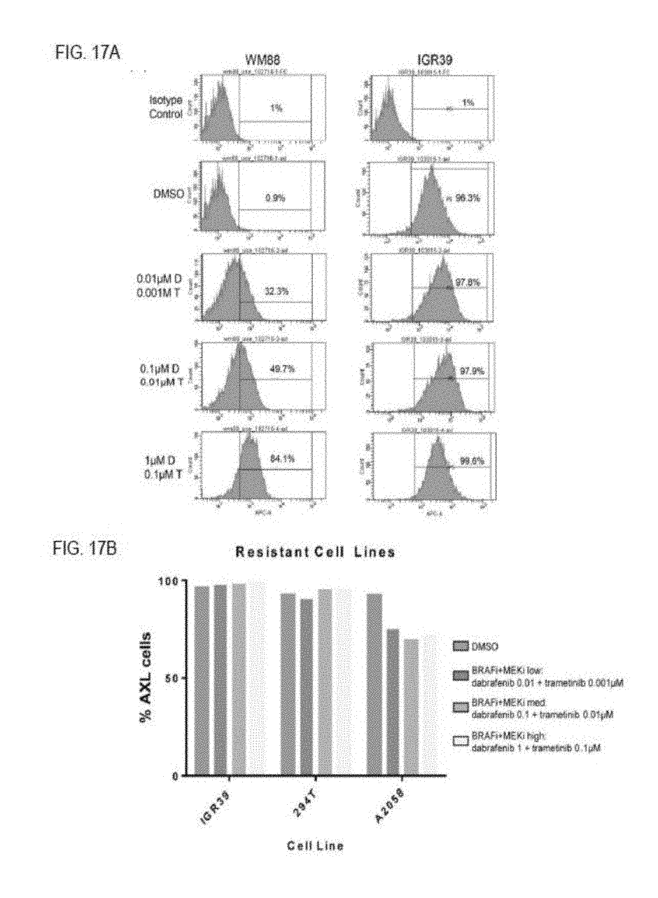

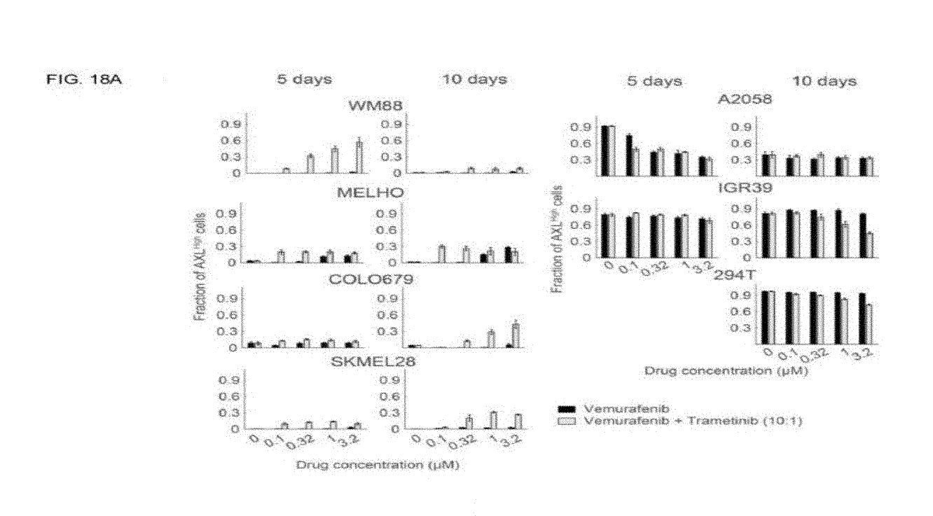

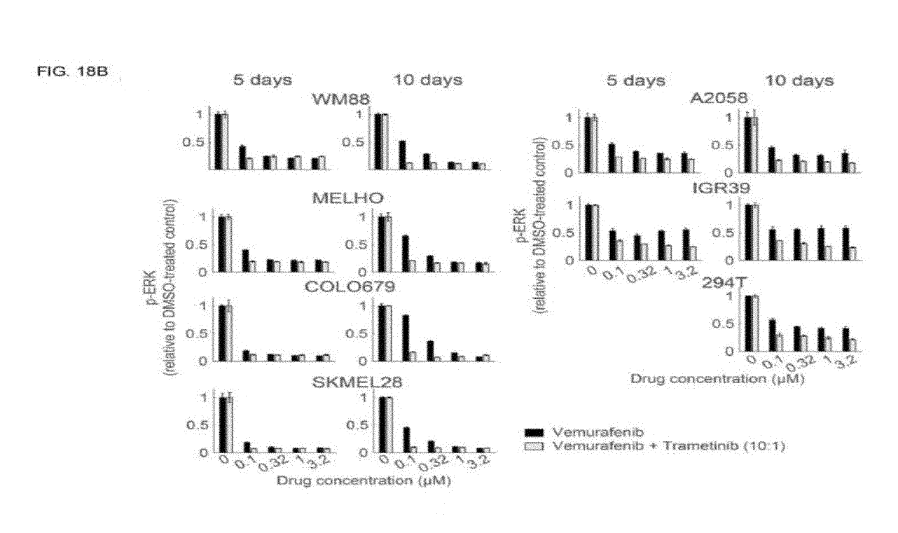

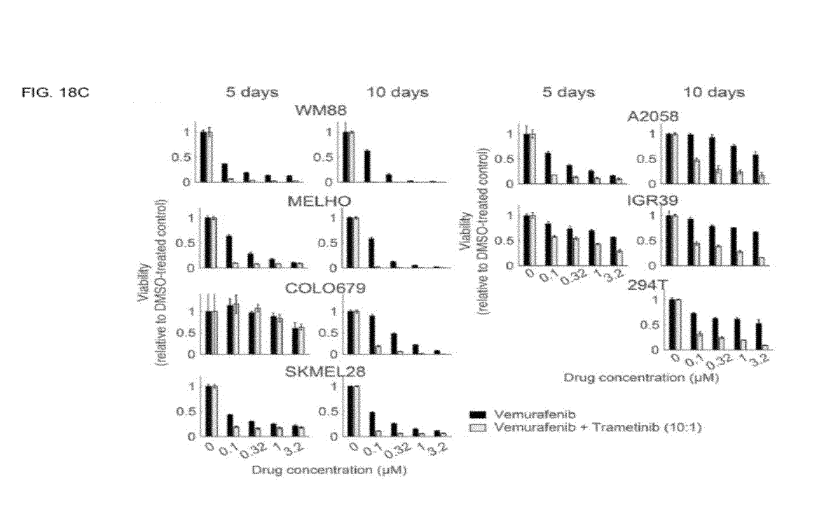

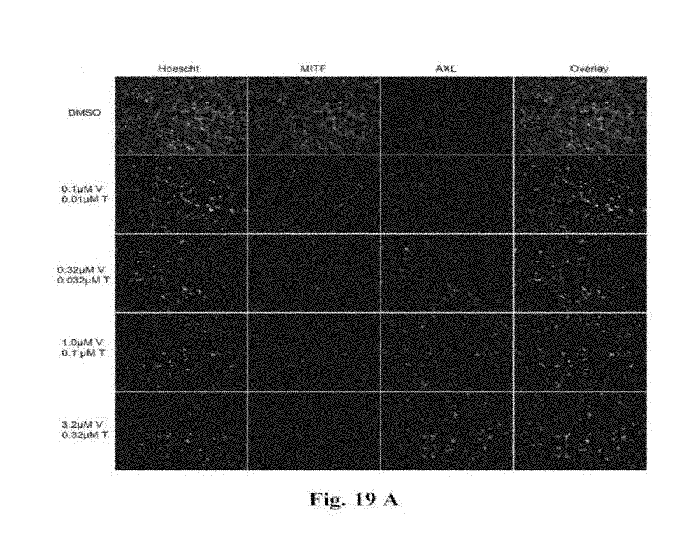

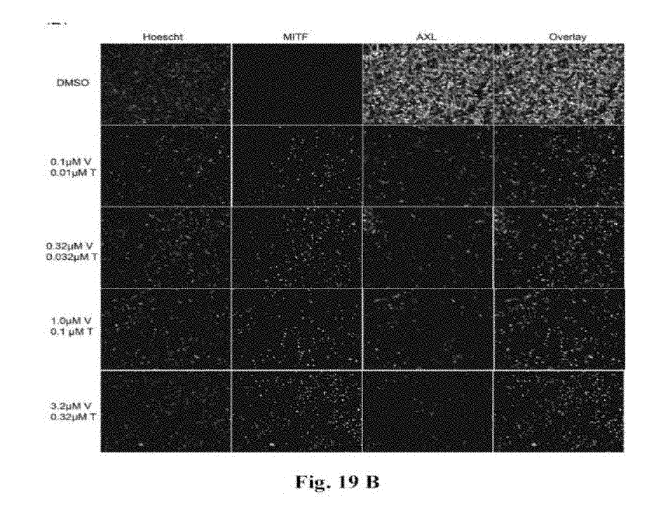

To further assess the connection between the AXL program and resistance to RAF/MEK inhibition, Applicants studied single-cell AXL expression in 18 melanoma cell lines from the CCLE (33) (Table 11). Flow-cytometry demonstrated a wide distribution of AXL-positive cells, from <1% to 99% per cell line, which correlated with bulk mRNA levels and were inversely associated with sensitivity to smallmolecule RAF inhibition (Table 11). Next, Applicants treated 10 cell lines (Methods) with increasing doses of a RAF/MEK inhibitor combination (dabrafenib and trametinib) (Methods) and found a rapid increase in the proportion of AXL-positive cells in six cell lines with a small (<3%) pre-treatment AXL-positive population (FIG. 3E; FIG. 17A). In cell line WM88, for example, the proportion of AXL-positive cells rose from ˜1% to 84% with BRAF/MEK-inhibition (FIG. 3E; FIG. 17-19). In contrast, cell lines with an intrinsically high proportion of AXL-expression, modest or no changes were observed (FIG. 17A,B). Similar results were obtained by multiplexed quantitative single-cell immunofluorescence (IF), which also demonstrated that the increased fraction of AXL-positive cells following RAF/MEK inhibition are associated with rapid decreases in ERK phosphorylation (reflecting MAP-kinase signaling inhibition) (FIG. 3F and FIG. 18-19). In summary, studies of both melanoma tumors and cell lines demonstrate that single-cell analysis can identify drug-resistant tumor cell subpopulations that become enriched during treatment with MAP-kinase targeted treatment.

TABLE 11
Characteristics of examined cell lines Cell line

| | MITF | | | Response to | | AXL |
|---|---|---|---|---|---|---|
| | mRNA | AXL mRNA | Vemurafenib | BRAF- | BRAF | expressing |
| | expression | expression | (IC50 μM) | inhbition | mutation | cells (%) |
| IGR39 | 7.65 | 10.77 | 8 | Resistant | BRAF V600E | 98 |
| LOXIMVI | 5.68 | 10.43 | 8 | Resistant | BRAF V600E/ | 97 |
| | | | | | I208V | |
| WM793 | 6.39 | 10.05 | 8 | Resistant | BRAF V600E | 99 |
| RPMI- | 6.2 | 9.78 | 8 | Resistant | BRAF V600E | 98 |
| 7951 | | | | | | |
| SKMEL24 | 7.36 | 9.74 | 5.15 | Resistant | BRAF V600E | 98 |
| A2058 | 8.71 | 9.63 | 8 | Resistant | BRAF V600E | 93 |
| Hs294T | 8.89 | 8.81 | 8 | Resistant | BRAF V600E | 93 |
| WM115 | 6.85 | 8.29 | 8 | Resistant | BRAF | 94 |
| | | | | | V600D | |
| IPC298 | 10.55 | 5.9 | 8 | Resistant | NRAS Q61L | 24 |
| SKMEL30 | 10.87 | 5.34 | 8 | Resistant | NRAS Q61K/ | 1 |
| | | | | | BRAF | |
| | | | | | D287H/ | |
| | | | | | E275K | |
| A375 | 7.64 | 9.33 | 0.26 | Sensitive | BRAF V600E | 96 |
| WM2664 | 10.43 | 8.19 | 1.58 | Sensitive | BRAF | 98 |
| | | | | | V600D | |
| WM88 | 10.05 | 6.39 | 0.2 | Sensitive | BRAF V600E | 1 |
| UACC62 | 9.5 | 5.85 | 0.25 | Sensitive | BRAF V600E | 2 |
| MELHO | 11.15 | 4.87 | 0.31 | Sensitive | BRAF V600E | 1 |
| SKMEL28 | 10.92 | 4.87 | | Sensitive | BRAF V600E | 3 |
| Colo679 | 10.34 | 4.83 | 0.55 | Sensitive | BRAF V600E | 0 |
| IGR37 | 10.85 | 4.73 | 0.9 | Sensitive | BRAF V600E | 1 |

MITF mRNA and AXL mRNA, vemurafenib IC50s and mutational status were extracted from CCLE (71). Cells were analyzed for the fraction of AXL-high cells using FACS. Cell lines highlighted in gray were subsequently used for treatment experiments and measurement of AXL-high fractions by flow-cytometry and multiplexed quantitative single-cell immunofluorescence analysis. Cell lines that are highlighted in gray were used for subsequent drug treatment experiments, flow-cytometry and single-cell immunofluorescence analysis.

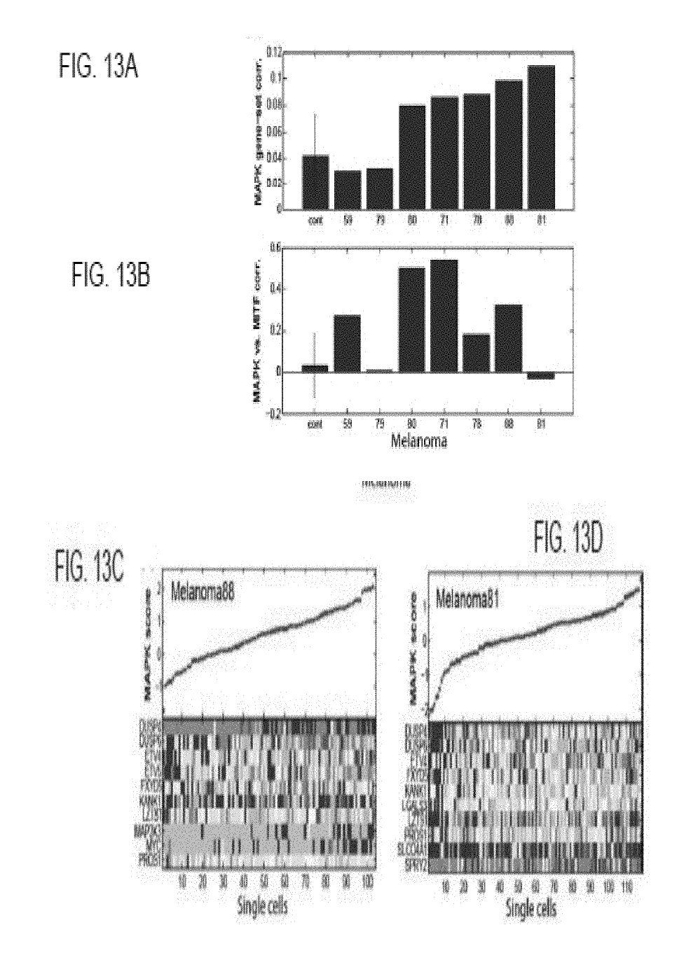

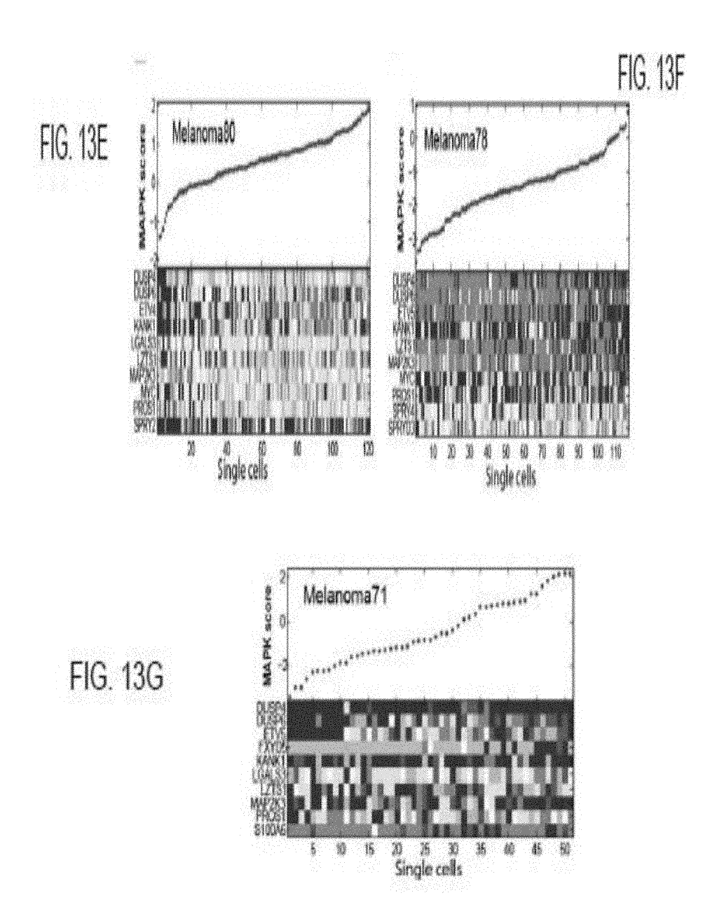

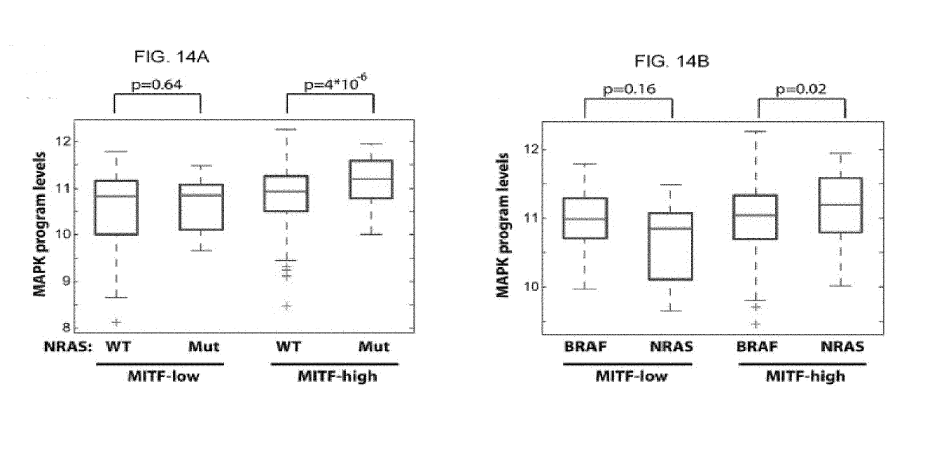

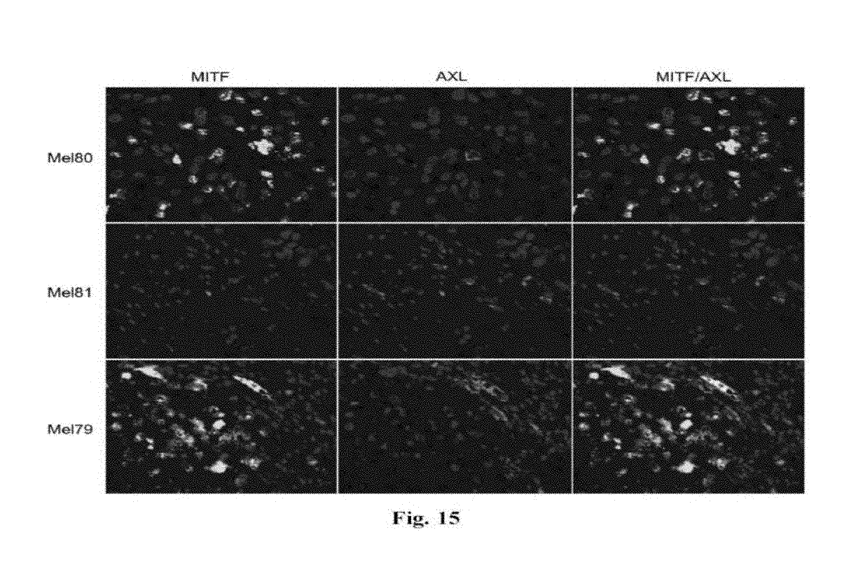

In principle, single-cell RNA-seq may also offer a categorical approach to quantify the outputs of oncogenic signal transduction. To test this idea in melanoma, where nearly all tumors exhibit genomic activation of MAP kinase signaling, Applicants interrogated a known signature of MAP kinase pathway activity across the individual malignant cells in the seven melanomas from our cohort with the largest number of malignant cells (FIG. 13). In five of these tumors the MAPK signature genes co-varied across cells, such that they correlated with one another more strongly than expected by chance (P<0.05 compared to 1000 randomly selected gene-sets), providing supporting evidence for variability of MAPK signaling within these tumors. This co-expression was particularly pronounced for a subset of MAPK signature genes, including the transcription factors ETV4/5 and regulators of the MAPK negative feedback DUSP4/6 and SPRY2/4. Expression of these genes was significantly low (P<0.05, t-test) in a subset of cells (4-18% of cells) in each of those five tumors, denoting a tumor cell subpopulation in which either MAPK signaling is inactive or alternatively the downstream response to MAP kinase signaling (e.g., the negative feedback arm) is low, such that these cells are relatively “indifferent” to the MAP kinase cascade. Three of these five tumors (CY71, CY80 and CY88) (Mel71, Mel80 and Mel88) carry an activating NRAS mutation and only in these tumors increased levels of the MAPK signature was significantly correlated (P<0.05) with the MITF-high expression program. Analysis of TCGA tumors further supported the connection between increased activity of the MITF program with the MAP kinase pathway in the context of NRAS mutant compared to NRAS wild-type or BRAF mutant melanoma (FIG. 14). Conceptually, measurement of oncogenic transcriptional output may inform us about pharmacodynamic properties in single tumor cells and provide a means of measuring target inhibition in genetically defined cancers treated with targeted therapies.

Non-Malignant Cells and their Interactions within the Melanoma Microenvironment

Various non-malignant cells comprise the tumor microenvironment. The composition of the microenvironment has an important impact on tumorigenesis and in the modulation of treatment responses. Tumor infiltration with T cells, for example, was found to be predictive for the response to immune checkpoint inhibitors in various cancer types (34).

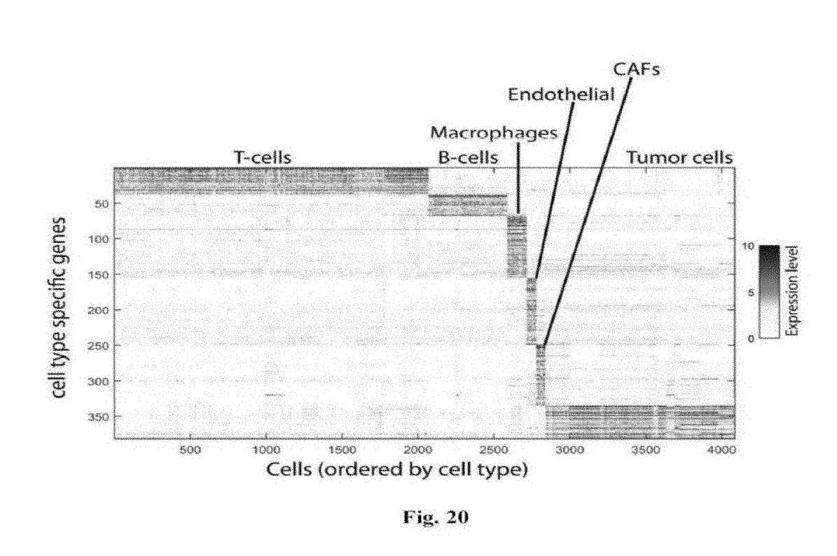

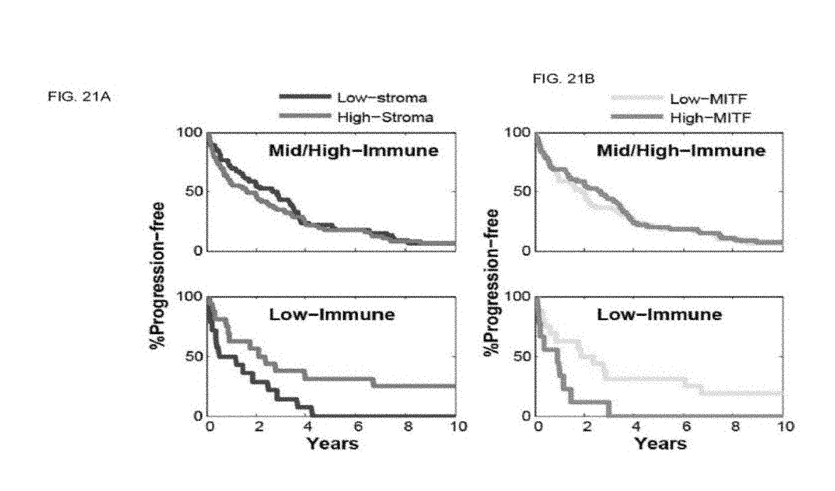

To resolve the composition of the melanoma microenvironment, Applicants first used our single-cell RNA-seq profiles to define unique expression signatures of each of five distinct non-malignant cell types: T cells, B cells, macrophages, endothelial cells, and CAFs. Because our signatures were derived from single cell profiles, Applicants could ensure that they are based on distinct genes for each cells type, avoiding confounders (Methods). Next, Applicants used these signatures to infer the relative abundance of those cell types in a larger compendium of tumors published recently by the TCGA consortium (Methods, FIG. 4A. FIG. 20). Supporting our strategy, Applicants found a strong correlation (R˜0.8) between our estimated tumor purity and that predicted from DNA analysis (35) (FIG. 4A, first lane below the heatmap).

Using this approach, Applicants partitioned the 495 TCGA tumors into 10 distinct microenvironment clusters based on their inferred cell type composition (FIG. 4A). For example, Cluster 9 consisted of tumors with a particularly high inferred content of B cells, whereas Cluster 4 had a relatively high inferred proportion of endothelial cells and CAFs. Clusters were mostly independent of the site of metastasis (FIG. 4A, second lane), with some notable exceptions (e.g., Clusters 8 and 9).

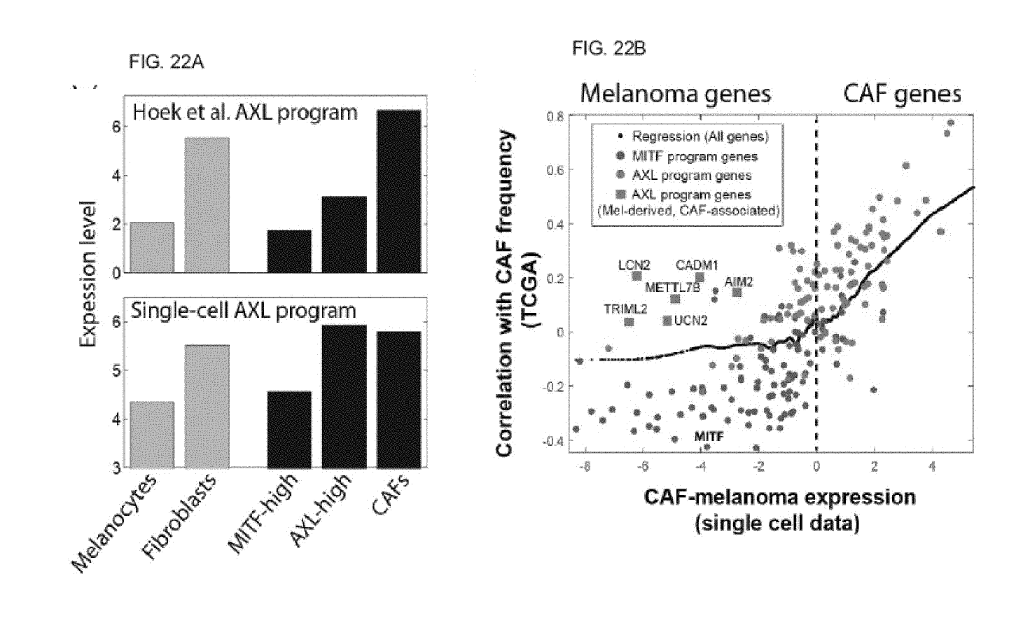

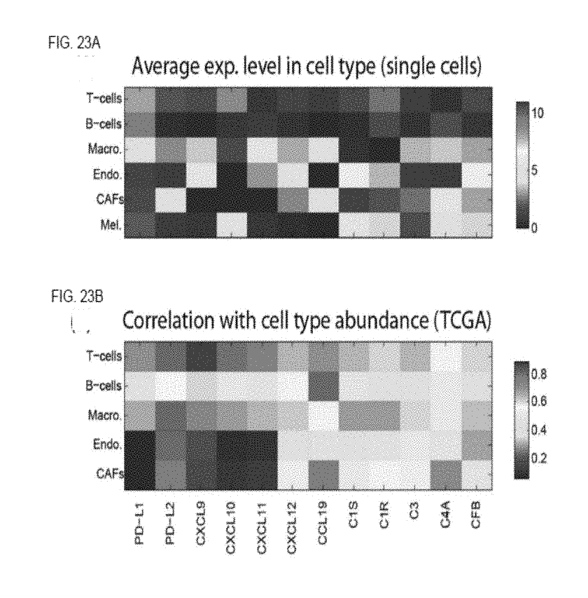

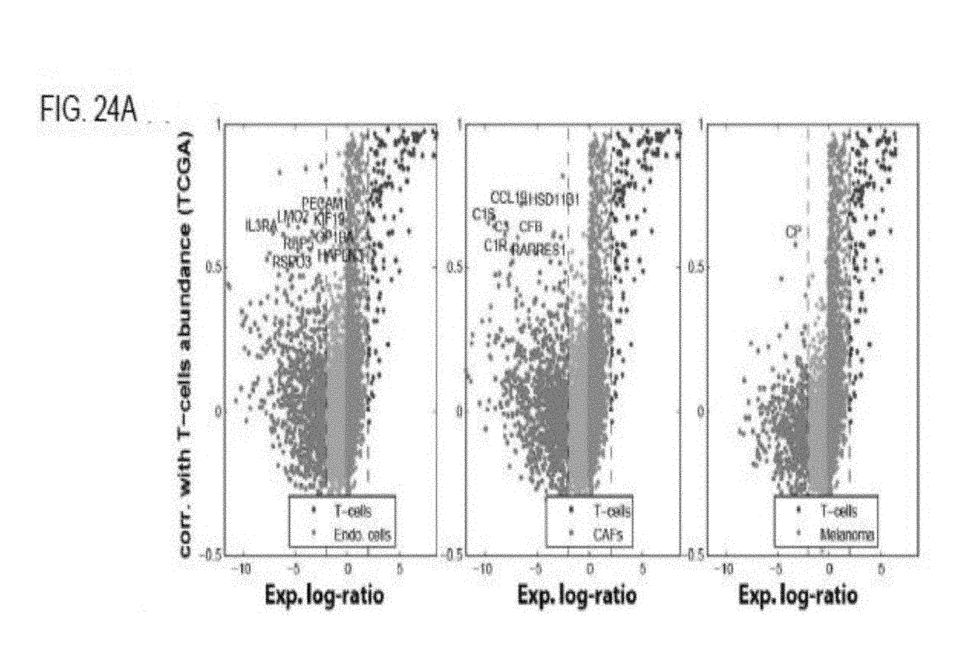

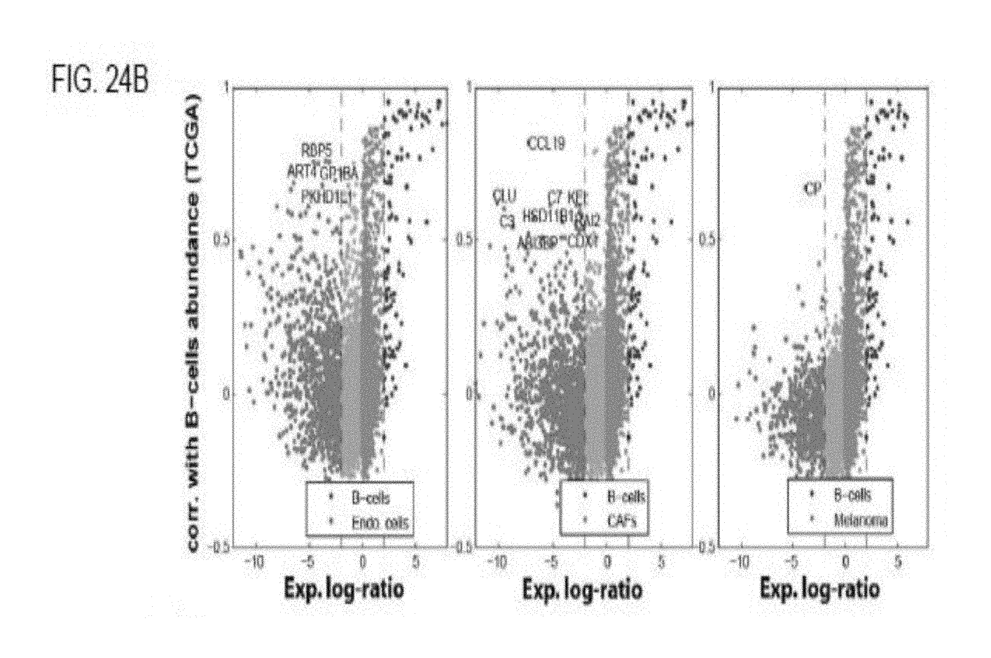

Next, Applicants examined how these different microenvironments may relate to the phenotype of the malignant cells. In particular, CAF abundance is predictive of the AXL-MITF distinction, such that CAF-rich tumors strongly expressed the AXL-high signature (FIG. 4A, bottom lane). Interestingly, an “AXL-high” program was expressed both by melanoma cells and by CAFs. However, using our single cell RNA-seq data. Applicants distinguished AXL-high genes that are preferentially expressed by melanoma cells (“melanoma-derived AXL program”) and those that are preferentially expressed by CAFs (“CAF derived AXL program”). Both sets of genes were correlated with the inferred CAF abundance in TCGA tumors (FIG. 22) (36). Furthermore, the MITF-high program, which is specific to melanoma cells, was negatively correlated with inferred CAF abundance. Taken together, these results suggest that CAF abundance may be linked to preferential expression of the AXL-high over the MITF-high program within the melanoma cells. Our findings raise the possibility that specific tumor-CAF interactions may shape the melanoma cell transcriptome.

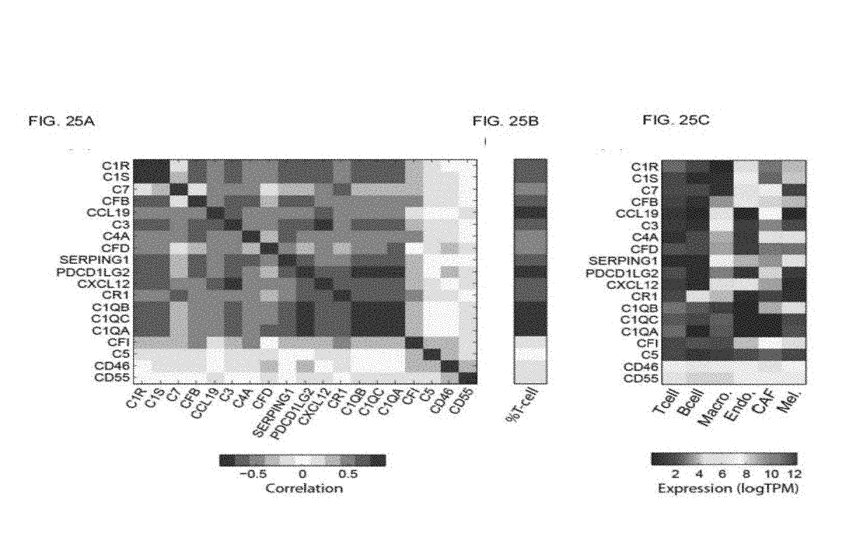

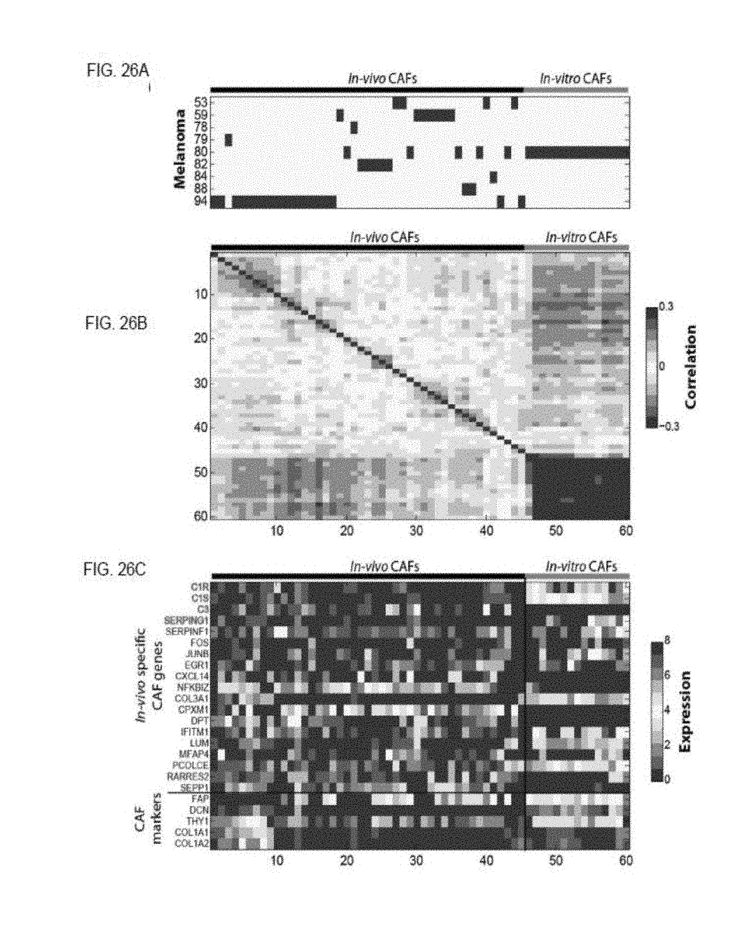

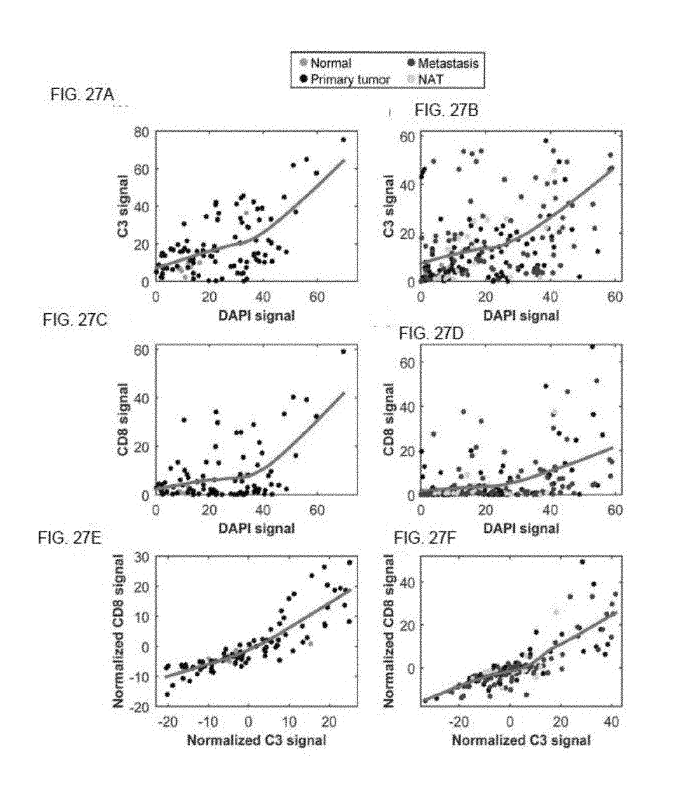

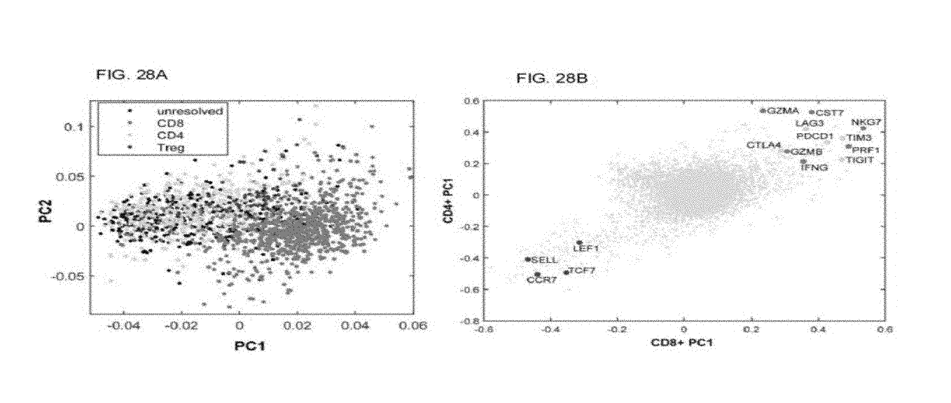

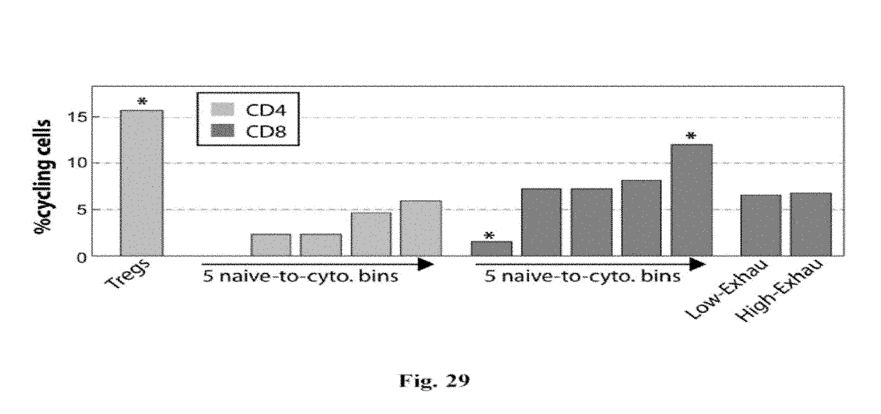

Interactions between cells play crucial roles in the tumor microenvironment. To assess systematically how cell-cell interactions may influence tumor composition, Applicants searched for genes expressed by cells of one type that may influence the proportion of cells of a different type in the tumor (FIG. 24). For example, Applicants searched for genes expressed primarily by CAFs (but not T cells) in single cell data that correlated with T cell abundance (as inferred by T cell specific genes) in bulk tumor tissue from the TCGA data set (37). Applicants identified a set of CAF-expressed genes that correlated strongly with T cell infiltration (FIG. 4B, red circles). These included known chemotactic (CXCL12, CCL19) and immune modulating (PD-L2) genes, which are significantly expressed by both CAFs and macrophages (FIG. 25). A separate set of genes exclusively expressed by CAFs that correlated with T cell infiltration (FIG. 25) included multiple complement factors (C1S, C1R, C3, C4A, CFB and C1NH [SERPING1]). Notably, these complement genes were specifically expressed by freshly isolated CAFs but not by cultured CAFs (FIG. 26) or macrophages (FIG. 25). These findings are intriguing in light of several studies that have implicated complement activity in the recruitment and modulation of T cell mediated anti-tumor immune responses (in addition to the established role of complement in innate immunity; (38)).

Applicants validated a high correlation (R>0.8) between complement factor 3 (C3) levels (one of the CAFexpressed complement genes) and infiltration of CD8+ T cells. To this end, Applicants performed dual IF staining and quantitative slide analysis of two tissue microarrays (TMAs) with a total of 308 core biopsies, including primary tumors, metastatic lesions, normal skin with adjacent tumor and healthy skin controls (FIG. 4C; FIG. 27, Methods). To test the generalizability of the association between CAF derived complement factors with T cell infiltration, Applicants expanded the analysis to bulk RNA-seq datasets across all TCGA cancer types (FIG. 4D). Consistent with the results in melanoma, complement factors correlated with inferred T cell abundance in many cancer types, and more highly than in normal tissues (e.g., R>0.4 for 65% of cancer types but only for 14% of normal tissue types). Although correlation analysis cannot determine causality, this indicates a potential in vivo role for cell-to-cell interactions.

Interestingly, the ‘tumor microenvironment clusters’ were also predictive of the dichotomy into MITF-high vs. AXL-high states in melanoma cells (FIG. 4A) and were linked to differences in the clinical outcomes (FIG. 21). In particular, CAF abundance in TCGA tumors was highly correlated with AXL-high expression patterns (FIG. 4A), due to two distinct effects. These observations suggest that the AXL-program is intrinsic to the fibroblast lineage, and is acquired by some melanoma malignant cells during carcinogenesis. Collectively, these results suggest that tumor-CAF interactions and/or CAF-induced remodeling of the microenvironment contribute to shaping the melanoma cell transcriptomes.

To uncover the basis of the association between different cell types in the tumor microenvironment clusters, Applicants next searched for factors expressed by non-malignant cells of one type but also influence the proportion of cells of a different type. In particular, Applicants searched for genes that were expressed primarily by CAFs in the single cell data but were also correlated with immune cell abundance (as inferred by T cell specific gene sets) in bulk tumor tissue in TCGA melanomas. Applicants found that a distinct subset of CAF-expressed genes correlated strongly with higher immune cell infiltration (FIG. 4E). These included known chemotactic (CXCL12, CCL19) and immune modulating genes (PD-L2), which are significantly expressed both by CAFs and by macrophages (FIG. 23). In addition, a set of genes strongly correlated with immune cell infiltration included multiple complement factors (C1S, C1R, C3, C4A and CFB) that were more exclusively expressed in CAFs (FIG. 23). Interestingly, the expression of these CAF-specific immune modulators and complement factors was relatively specific to in vivo CAFs compared to single-cell transcriptomes of short-term patient-derived CAF cultures and in comparison to normal foreskin fibroblasts. This highlights the influence of the melanoma microenvironment on tumor composition and stresses the importance of directly analyzing fresh patient-derived cells over cell cultures. In addition to the established role of complement in innate immunity, several studies have implicated complement activity in the recruitment and suppression of T cell mediated anti-tumor immune responses. Overall, this analysis suggests stroma-derived and immune-derived mechanisms that may regulate the recruitment or proliferation of immune cells, and thus targeting these components of the complement system or these cytokines could be a therapeutic avenue.

Diversity of Tumor-Infiltrating T Lymphocytes and their Functional States

The activity of tumor-infiltrating lymphocytes (TILs)—in particular CD8+ T cells—is a major determinant of successful immune surveillance. Under normal circumstances, effector CD8+ T cells exposed to antigens and co-stimulatory factors mediate lysis of malignant cells and control tumor growth. However, this function can be hampered by tumor-mediated T cell exhaustion, such that T cells fail to activate cytotoxic effector functions (39). Exhaustion is promoted through the stimulation of coinhibitory “checkpoint” molecules on the T cell surface (PD-1, TIM-3, CTLA-4, TIGIT, LAG3 and others) (40): blockade of checkpoint mechanisms has shown remarkable clinical benefit in subsets of melanoma and other malignancies (3, 10, 41, 42). While checkpoint ligand expression (e.g., PD-L1) and neoantigen load clearly contribute (9, 43, 44), no biomarker has emerged that reliably predicts the clinical response to immune checkpoint blockade. Applicants reasoned that single cell analyses might yield features that can be used in the future to elucidate response determinants and possibly identify new immunotherapy targets.

To characterize this diversity in human tumors, Applicants analyzed the single-cell expression patterns of 2,068 T cells from 15 melanomas. Applicants first identified T cells and their main subsets (CD4+, Tregs, and CD8+) based on the expression levels of their respective defining surface markers (FIG. 5A, top and Table 12). Within both the CD4+ and CD8+ populations, a principal component analysis distinguished cell subsets and heterogeneity of activation states based on expression of naïve and cytotoxic T cell genes (FIG. 5A-B and FIG. 28).

TABLE 12
Genes preferentially expressed by Tregs compared
to CD4+ and CD8+ T-cells

| | Tregs/CD4+ | | Tregs/CD8+ | |
|---|---|---|---|---|
| | | significance | | significance |
| Gene Name | log2-ratio | (−log10(P)) | log2-ratio | (−log10(P)) |
| IL2RA | 4.9314 | 108.0864 | 4.9429 | 156.3565 |
| FOXP3 | 4.203 | 89.2082 | 4.3284 | 196.1143 |
| S100A4 | 3.4739 | 10.3922 | 3.6712 | 12.825 |
| CCR8 | 3.4462 | 34.0957 | 3.6126 | 100.6657 |
| TNFRSF1B | 3.3038 | 14.9444 | 2.4584 | 9.0528 |
| GBP5 | 3.2691 | 21.9609 | 1.994 | 7.2986 |
| TNFRSF18 | 3.1395 | 13.1937 | 3.8084 | 39.3184 |
| IFI6 | 3.1378 | 10.4917 | 2.4915 | 7.0957 |
| CXCR6 | 2.8035 | 11.1341 | 1.2444 | 1.8837 |
| PIM2 | 2.783 | 9.7392 | 3.6418 | 19.0767 |
| LGALS1 | 2.7658 | 10.2398 | 2.1396 | 6.2732 |
| BATF | 2.7427 | 8.9412 | 2.9111 | 11.5239 |
| TNFRSF4 | 2.7405 | 11.0809 | 3.724 | 67.4286 |
| GBP2 | 2.6039 | 8.5013 | 2.0545 | 5.6399 |
| S100A6 | 2.4478 | 7.2581 | 1.853 | 4.9506 |
| UGP2 | 2.4448 | 9.5419 | 2.6079 | 12.8918 |
| CTSC | 2.4278 | 14.0409 | 2.1092 | 10.6288 |
| SAT1 | 2.411 | 6.4101 | 2.5169 | 7.0602 |
| IL32 | 2.4067 | 10.6603 | 2.0114 | 10.4194 |
| APOBEC3C | 2.384 | 6.8456 | 0.3962 | 0.3762 |
| IL2RB | 2.3507 | 10.0447 | 1.3959 | 4.1239 |
| CTLA4 | 2.2923 | 8.1621 | 2.226 | 9.679 |
| ENO1 | 2.2681 | 6.577 | 2.6227 | 8.4014 |
| ACP5 | 2.2576 | 8.6929 | 1.5582 | 3.7963 |
| SELPLG | 2.2563 | 6.2061 | 2.5352 | 8.7096 |
| COX17 | 2.2174 | 10.9203 | 1.8901 | 7.6237 |
| CCND2 | 2.1527 | 10.5771 | 1.3008 | 3.7349 |
| PRDX3 | 2.1424 | 8.6678 | 1.4985 | 3.8471 |
| LAIR2 | 2.1415 | 13.851 | 2.0799 | 15.8578 |
| LTB | 2.1273 | 4.2022 | 4.7733 | 34.5617 |
| PRDM1 | 2.1105 | 8.2645 | 1.4024 | 4.2404 |
| HSPA1A | 2.0835 | 5.9936 | −0.2198 | 0.1588 |
| IL10RA | 2.0721 | 5.9976 | 1.1443 | 2.1226 |
| PRNP | 2.0648 | 6.5277 | 2.5922 | 13.0264 |
| TYMP | 2.0431 | 15.7423 | 1.5948 | 7.2617 |
| NDUFA13 | 2.0129 | 5.016 | 1.8961 | 4.5219 |
| SYNGR2 | 1.9999 | 5.7351 | 1.3058 | 2.5734 |
| SQSTM1 | 1.9941 | 7.2362 | 1.6929 | 5.4276 |
| STAT1 | 1.9898 | 4.858 | 1.733 | 3.7968 |
| LINC00152 | 1.9851 | 6.3335 | 0.9553 | 1.7154 |
| CD27 | 1.9849 | 4.1972 | 0.6058 | 0.7365 |
| CXCR3 | 1.98 | 5.3375 | 1.6348 | 4.0588 |
| TIGIT | 1.9668 | 4.6304 | 0.6416 | 0.8306 |
| MRPS6 | 1.9596 | 6.3062 | 1.9272 | 6.9118 |
| CLIC1 | 1.9249 | 4.5393 | 1.2696 | 2.3622 |
| PARK7 | 1.9208 | 4.2626 | 1.2864 | 2.1789 |
| CD74 | 1.92 | 4.7128 | −0.1704 | 0.202 |
| SDC4 | 1.8928 | 17.7383 | 1.775 | 16.7533 |
| SOD1 | 1.8784 | 4.6144 | 1.5636 | 3.4375 |
| FTL | 1.8447 | 5.5337 | 1.0957 | 2.5111 |
| ISG15 | 1.8244 | 3.5101 | 1.4318 | 2.4338 |
| LY6E | 1.7697 | 4.5628 | 1.3713 | 3.0396 |
| DUSP4 | 1.7572 | 5.7029 | −0.1149 | 0.1174 |
| GCHFR | 1.7485 | 7.5737 | 1.5724 | 6.2974 |
| TPM4 | 1.7445 | 4.8499 | 2.1814 | 8.719 |
| PRF1 | 1.7444 | 6.3169 | −2.1843 | 5.3341 |
| ACTN4 | 1.7392 | 7.4175 | 0.7837 | 1.5797 |
| ANKRD10 | 1.7306 | 5.9561 | 1.4854 | 4.7378 |
| FAM110A | 1.7248 | 8.838 | 1.7443 | 11.1629 |
| COX5A | 1.7214 | 4.2827 | 1.5293 | 3.3323 |
| CST7 | 1.6971 | 3.5333 | −2.2012 | 6.2886 |
| GABARAP | 1.691 | 4.0968 | 1.6808 | 4.0383 |
| PHLDA1 | 1.6828 | 11.0367 | 0.9662 | 3.0102 |
| SUMO2 | 1.6769 | 3.9712 | 1.8155 | 4.5819 |
| TAP1 | 1.6768 | 3.7399 | 0.6796 | 0.921 |
| VCP | 1.6724 | 4.3504 | 1.7534 | 5.0804 |
| ICOS | 1.6511 | 3.1124 | 2.5341 | 8.9582 |
| C17orf49 | 1.6435 | 4.1573 | 1.2955 | 2.595 |
| IL2RG | 1.6364 | 3.9312 | 1.4064 | 3.0846 |
| BUB3 | 1.6249 | 3.8154 | 0.8231 | 1.2816 |
| PEBP1 | 1.5804 | 3.3888 | 1.6761 | 4.1517 |
| PLP2 | 1.5799 | 3.9804 | 1.4823 | 3.7429 |
| LSP1 | 1.5742 | 3.1647 | 0.6289 | 0.8449 |
| NAMPT | 1.5693 | 7.2891 | 1.7405 | 11.5589 |
| CRADD | 1.5687 | 11.3383 | 1.6363 | 20.1184 |
| ATP6V0E1 | 1.567 | 3.0378 | 1.8802 | 4.0639 |
| PRDX6 | 1.562 | 4.886 | 1.1606 | 2.7899 |
| SPPL2A | 1.5464 | 4.9576 | 1.4549 | 4.7904 |
| PSMB3 | 1.5383 | 2.8248 | 1.2727 | 2.1416 |
| BST2 | 1.5219 | 3.6094 | 1.0841 | 1.9052 |
| SLAMF1 | 1.5193 | 4.5894 | 2.282 | 19.8918 |
| CRIP1 | 1.5172 | 2.6247 | 0.9933 | 1.423 |
| CSF1 | 1.507 | 9.8658 | 0.8546 | 2.475 |
| DUSP16 | 1.5059 | 8.837 | 1.4756 | 10.197 |
| LGALS3 | 1.5045 | 4.0982 | 1.4202 | 4.2955 |
| OTUB1 | 1.4974 | 4.3779 | 1.584 | 4.9134 |
| PDIA6 | 1.4971 | 4.0511 | 0.7905 | 1.2344 |
| GABARAPL2 | 1.491 | 3.595 | 1.4439 | 3.4709 |
| GLRX | 1.4862 | 3.8439 | 1.8348 | 6.5624 |
| CD7 | 1.4846 | 6.6389 | 0.4425 | 0.7692 |
| IL1R2 | 1.4826 | 12.7171 | 1.554 | 35.0035 |
| TPI1 | 1.4791 | 2.4408 | 0.8294 | 1.0138 |
| MX1 | 1.4784 | 5.0034 | 1.1599 | 3.1162 |
| PBXIP1 | 1.4711 | 4.141 | 2.8843 | 20.6602 |
| HLA-DPA1 | 1.4666 | 3.4947 | −1.4391 | 2.5483 |
| OAS1 | 1.464 | 5.6234 | 1.3653 | 5.4415 |
| FBXW5 | 1.4636 | 4.5146 | 1.5089 | 5.6328 |
| ANXA2 | 1.4608 | 2.6396 | 1.3945 | 2.6863 |
| RTKN2 | 1.4583 | 18.869 | 1.5568 | 51.7679 |
| LASP1 | 1.4533 | 4.1449 | 1.2308 | 3.2262 |
| TNFRSF9 | 1.4497 | 11.6612 | −0.1722 | 0.2282 |
| WDR1 | 1.448 | 3.6362 | 1.4179 | 3.6517 |
| SH2D2A | 1.4454 | 4.9413 | 0.9791 | 2.4114 |
| MYL6 | 1.4434 | 4.2888 | 1.3482 | 3.5196 |
| ACAA1 | 1.4389 | 4.0391 | 1.5627 | 5.6314 |
| NOP10 | 1.4334 | 3.3827 | 1.078 | 2.0201 |
| DPYSL2 | 1.4279 | 8.1775 | 1.477 | 11.114 |
| PSMD2 | 1.4239 | 4.1145 | 1.25 | 3.3147 |
| CCR5 | 1.4169 | 4.3057 | 0.3008 | 0.3365 |
| HAPLN3 | 1.4067 | 4.509 | 1.6356 | 7.8559 |
| COX6B1 | 1.3985 | 2.9477 | 1.304 | 2.7498 |
| MYO1G | 1.3971 | 4.5973 | 0.7691 | 1.4872 |
| CTSA | 1.3948 | 3.7213 | 1.5284 | 4.7298 |
| CALM3 | 1.3864 | 4.6899 | 0.9947 | 2.6976 |
| PTPN7 | 1.3846 | 3.1375 | 0.707 | 1.0896 |
| CTNNB1 | 1.3846 | 4.5104 | 1.1333 | 3.2912 |
| PHTF2 | 1.384 | 4.0246 | 2.2315 | 14.1826 |
| PSMB1 | 1.3829 | 2.2889 | 1.7349 | 3.5906 |
| ATP5B | 1.3802 | 2.4225 | 1.4684 | 2.7511 |
| ARRDC1 | 1.371 | 4.1943 | 1.2726 | 3.7427 |
| PTTG1 | 1.3517 | 3.4075 | 1.2953 | 3.4109 |
| TPP1 | 1.3507 | 3.2258 | 1.8232 | 6.3944 |
| ISG20 | 1.3489 | 2.5137 | 1.2107 | 2.0813 |
| TWF2 | 1.3486 | 3.2437 | 1.1262 | 2.3436 |
| EID1 | 1.3459 | 3.2424 | 0.9325 | 1.7275 |
| ATP5E | 1.3441 | 2.8331 | 0.6234 | 1.0373 |
| ARPC1B | 1.3416 | 2.5386 | 1.8015 | 4.0743 |
| NDUFB8 | 1.3414 | 2.4351 | 0.8999 | 1.294 |
| SHMT2 | 1.3395 | 4.7184 | 1.4804 | 7.3149 |
| TUBB | 1.3374 | 2.4108 | 1.0608 | 1.6405 |
| HLA-DRB1 | 1.3265 | 3.3234 | −1.6063 | 3.6511 |
| DDB2 | 1.3116 | 4.3634 | 1.416 | 5.6489 |
| TANK | 1.3091 | 3.1295 | 1.2604 | 3.0242 |
| NCF4 | 1.3041 | 4.484 | 1.8421 | 21.6217 |
| TMEM60 | 1.2997 | 5.1834 | 1.3407 | 7.5323 |
| PSMA1 | 1.2991 | 2.5203 | 1.4163 | 3.0406 |
| TCEB2 | 1.293 | 3.1752 | 1.2509 | 3.0595 |
| APOBEC3G | 1.2918 | 2.9403 | −1.118 | 1.7578 |
| ARHGAP9 | 1.2876 | 3.1194 | 0.8446 | 1.5337 |
| SERPINB9 | 1.2814 | 3.5861 | 0.5383 | 0.8663 |
| CMC2 | 1.2791 | 3.325 | 1.2574 | 3.3681 |
| WSB1 | 1.2712 | 3.8498 | 1.1098 | 3.0142 |
| PLD3 | 1.2689 | 5.2576 | 1.264 | 5.76 |
| GPS2 | 1.2629 | 2.9045 | 1.2236 | 3.0433 |
| OCIAD2 | 1.2578 | 2.444 | 1.6864 | 4.5153 |
| SNX5 | 1.2562 | 3.7595 | 1.248 | 3.7184 |
| DGUOK | 1.2562 | 3.185 | 1.2082 | 3.1996 |
| IKZF2 | 1.2556 | 10.2888 | 1.1321 | 9.9732 |
| GPX1 | 1.2503 | 2.278 | 2.0277 | 7.8061 |
| PTPN1 | 1.25 | 4.3921 | 1.1973 | 4.4626 |
| VDR | 1.2404 | 9.2804 | 1.1793 | 9.6917 |
| SAMD9 | 1.2355 | 6.636 | 0.8628 | 2.9563 |
| RAC2 | 1.2345 | 2.4824 | 1.2087 | 2.4981 |
| RPS27L | 1.2258 | 3.8407 | 1.4026 | 5.5632 |
| EPS15 | 1.2232 | 4.1322 | 1.1412 | 3.9182 |
| CAP1 | 1.2229 | 2.6631 | 1.2053 | 2.6106 |
| AP2M1 | 1.2219 | 2.5587 | 1.0708 | 2.1636 |
| NDUFB10 | 1.2218 | 2.5617 | 0.9597 | 1.6679 |
| AGTRAP | 1.2206 | 4.0087 | 1.2162 | 4.5654 |
| IRF9 | 1.2192 | 2.3886 | 0.5484 | 0.6954 |
| HLA-DMA | 1.2021 | 4.5233 | −0.7323 | 1.0207 |
| MAGEH1 | 1.1986 | 2.9482 | 1.7923 | 11.8359 |
| TMED9 | 1.1941 | 2.2484 | 1.3405 | 3.0532 |
| TFRC | 1.1938 | 4.0512 | 1.1977 | 4.2677 |
| EMP3 | 1.1936 | 2.3379 | 1.5454 | 3.9512 |
| RHOF | 1.1931 | 2.8382 | 1.3896 | 3.8433 |
| PGK1 | 1.193 | 2.1025 | 1.0509 | 1.8193 |
| CAST | 1.1865 | 4.0358 | 1.2894 | 5.0711 |
| CD58 | 1.1837 | 2.8965 | 1.2941 | 3.6738 |
| NDUFV2 | 1.1791 | 2.0201 | 1.5293 | 3.417 |
| CD79B | 1.1785 | 3.4684 | 1.3654 | 5.5062 |
| PAIP2 | 1.1768 | 2.1353 | 1.0782 | 1.8948 |
| TARDBP | 1.1747 | 3.3346 | 1.0885 | 2.9811 |
| SFT2D1 | 1.1747 | 2.5526 | 0.8662 | 1.5283 |
| STAM | 1.1737 | 4.6628 | 1.491 | 11.2261 |
| GBP4 | 1.1683 | 5.7353 | 0.759 | 2.3531 |
| HPRT1 | 1.1606 | 4.0411 | 0.9824 | 2.8081 |
| TMSB10 | 1.1575 | 5.6919 | 1.2878 | 6.425 |
| U2AF1L4 | 1.1552 | 3.9465 | 0.9408 | 2.7047 |
| TPM3 | 1.1527 | 3.6936 | 1.2356 | 4.1502 |
| C3AR1 | 1.1519 | 8.6292 | 1.1896 | 14.5168 |
| CDKN1B | 1.1507 | 2.8125 | 0.7531 | 1.3981 |
| TMEM173 | 1.1454 | 2.149 | 1.802 | 5.8798 |
| TRAPPC1 | 1.1423 | 3.2075 | 1.1024 | 3.1881 |
| RAP1A | 1.1422 | 2.9078 | 1.2535 | 3.847 |
| NFKBIZ | 1.1405 | 2.7426 | 1.6435 | 6.4682 |
| HERPUD1 | 1.1375 | 2.1122 | 0.8367 | 1.3027 |
| FKBP1A | 1.1366 | 2.1013 | 0.8428 | 1.3552 |
| B4GALT1 | 1.1362 | 3.546 | 1.2567 | 4.9898 |
| EIF4A1 | 1.1359 | 2.0004 | 1.271 | 2.4293 |
| OTUD5 | 1.1356 | 4.8059 | 1.2142 | 6.3012 |
| IRF2 | 1.1321 | 3.5988 | 0.3738 | 0.5464 |
| CCR4 | 1.1316 | 2.2499 | 2.2758 | 23.2853 |
| RHOC | 1.1306 | 3.0064 | 0.7756 | 1.5918 |
| ADORA2A | 1.1301 | 4.2427 | 0.6748 | 1.3801 |
| MRPL36 | 1.1285 | 4.8562 | 0.9545 | 3.3227 |
| PMAIP1 | 1.1283 | 3.3635 | 0.4399 | 0.6228 |
| RNF213 | 1.1278 | 5.5662 | 0.7493 | 3.1218 |
| REREP3 | 1.1263 | 4.3411 | 1.5126 | 23.4758 |
| ARPC5L | 1.1254 | 2.565 | 0.5489 | 0.7658 |
| VDAC2 | 1.123 | 2.2417 | 1.1622 | 2.5702 |
| HSD17B10 | 1.1222 | 2.5763 | 1.311 | 4.0266 |
| PELI1 | 1.1215 | 3.9849 | 1.3548 | 7.7508 |
| MRPS7 | 1.1196 | 2.974 | 1.076 | 2.9395 |
| GNPTAB | 1.1181 | 6.5425 | 0.9386 | 4.3756 |
| YWHAE | 1.1092 | 2.9974 | 0.689 | 1.253 |
| ATP6V1E1 | 1.1076 | 2.5331 | 0.9287 | 1.9102 |
| GALM | 1.107 | 3.0304 | 0.7437 | 1.4177 |
| ERI1 | 1.1069 | 7.1931 | 1.2037 | 11.6122 |
| BANF1 | 1.1031 | 3.3315 | 0.8063 | 1.8427 |
| SAMSN1 | 1.102 | 2.2355 | 1.2736 | 3.134 |
| TXN | 1.1018 | 2.8026 | 1.0062 | 2.5035 |
| PRDX5 | 1.0999 | 2.0767 | 0.5756 | 0.7511 |
| PTP4K2C | 1.0991 | 3.5209 | 1.1964 | 4.7433 |
| CMTM7 | 1.096 | 2.2708 | 1.4967 | 5.2249 |
| FCRL3 | 1.0957 | 4.8266 | −0.8363 | 1.463 |
| COX7A2L | 1.0953 | 2.0561 | 1.2282 | 2.7693 |
| GNG5 | 1.0911 | 2.0219 | 0.9472 | 1.7154 |
| ACTR1A | 1.0874 | 3.2474 | 1.0875 | 3.6302 |
| APLP2 | 1.0855 | 3.9035 | 0.9113 | 3.0437 |
| CSF2RB | 1.0854 | 11.8913 | 1.1409 | 33.281 |
| EXOSC7 | 1.0825 | 3.6053 | 1.0241 | 3.4395 |
| CACYBP | 1.082 | 2.974 | 0.717 | 1.4253 |
| PPP2R1A | 1.0791 | 2.1016 | 1.0792 | 2.1817 |
| MGAT1 | 1.0713 | 2.5957 | 0.8291 | 1.6717 |
| OVCA2 | 1.0697 | 2.9705 | 0.8743 | 2.0155 |
| UBA1 | 1.069 | 2.4156 | 1.2125 | 3.1312 |
| REC8 | 1.0664 | 5.4073 | 0.9344 | 4.2368 |
| KCNN4 | 1.0573 | 5.442 | 0.9763 | 4.7937 |
| ARHGEF6 | 1.0563 | 2.734 | 1.6628 | 8.1901 |
| RFK | 1.0544 | 5.8307 | 1.126 | 11.0342 |
| HTATIP2 | 1.0401 | 3.723 | 0.8485 | 2.3564 |
| ANXA11 | 1.0358 | 2.3683 | 1.0522 | 2.5286 |
| MAPKAPK3 | 1.0335 | 3.269 | 1.1717 | 5.0343 |
| SNX10 | 1.0335 | 6.1494 | 0.9935 | 6.6335 |
| PSMA5 | 1.0241 | 2.7636 | 0.9663 | 2.4943 |
| BIRC3 | 1.0224 | 2.5934 | 1.3975 | 5.2056 |
| NDUFA3 | 1.0207 | 2.2145 | 0.7994 | 1.5508 |
| GATA3 | 1.017 | 3.9346 | 1.0305 | 4.1607 |
| SDF4 | 1.0169 | 2.6697 | 1.3371 | 5.3809 |
| UBE2B | 1.0132 | 2.8088 | 1.0963 | 3.5892 |
| NEMF | 1.013 | 3.287 | 0.8904 | 2.6344 |
| NDUFA11 | 1.002 | 2.1448 | 0.8833 | 1.7486 |
| SDF2L1 | 1.002 | 2.9401 | 0.7455 | 1.6546 |

All genes were significantly higher expressed (P < 0.01, fold-change > 2) in Tregs compared to other CD4+ T-cells.
Genes were sorted by fold-change increase its T-regs compared to other CD4+ T-cells, as shown in the second column.
Fourth and fifth columns contain the log-ratio and p-value in comparison of Tregs to CD8+ T-cells; this comparison was not used to define the gene-list but is provided as additional information

Next, Applicants aimed to determine the exhaustion status of each cell, based on the expression of key coinhibitory receptors (PD1, TIGIT, TIM3, LAG3 and CTLA4). In several cases, these co-inhibitory receptors were co-expressed across individual cells; Applicants validated this phenomenon for PD1 and TIM3 by immunofluorescence (FIG. 5C). However, exhaustion gene expression was also highly correlated with the expression of both cytotoxicity markers and overall T cell activation states (FIG. 5B). This observation resembles an “activation-dependent exhaustion expression program” previously reported in models of chronic viral infections (45). Accordingly, expression of co-inhibitory receptors (alone or in combinations) per se may not be sufficient to characterize the salient functional state of tumorassociated T lymphocytes in situ or to distinguish exhaustion from activation.

To define an “activation-independent exhaustion program”, Applicants leveraged single-cell data from a large number of CD8+ T cells sequenced in a single tumor (Mel75, 314 cells). These data allowed tumor cytotoxic and exhaustion programs to be deconvolved. Specifically, PCA of Mel75 T cell transcriptomes identified a robust expression module that consisted of all five co-inhibitory receptors and other exhaustion-related genes, but not cytotoxicity genes (FIG. 31 and Table 13).

Applicants then used the Mel75 exhaustion program, as well as two previously published exhaustion programs (45, 46) to estimate the exhaustion state of each cell. Here, exhaustion state was defined as “high” or “low” expression of the exhaustion program relative to that of cytotoxicity genes (FIG. 5D, Methods). Accordingly, Applicants defined exhaustion states in Mel75 and in four additional tumors with the highest number of CD8+ T cells (68 to 214 cells per tumor). Applicants then identified the top genes that were preferentially expressed in high-exhaustion compared to low-exhaustion cells (both defined relative to the expression of cytotoxicity genes). Finally, Applicants defined a core exhaustion signature across cells from various tumors.

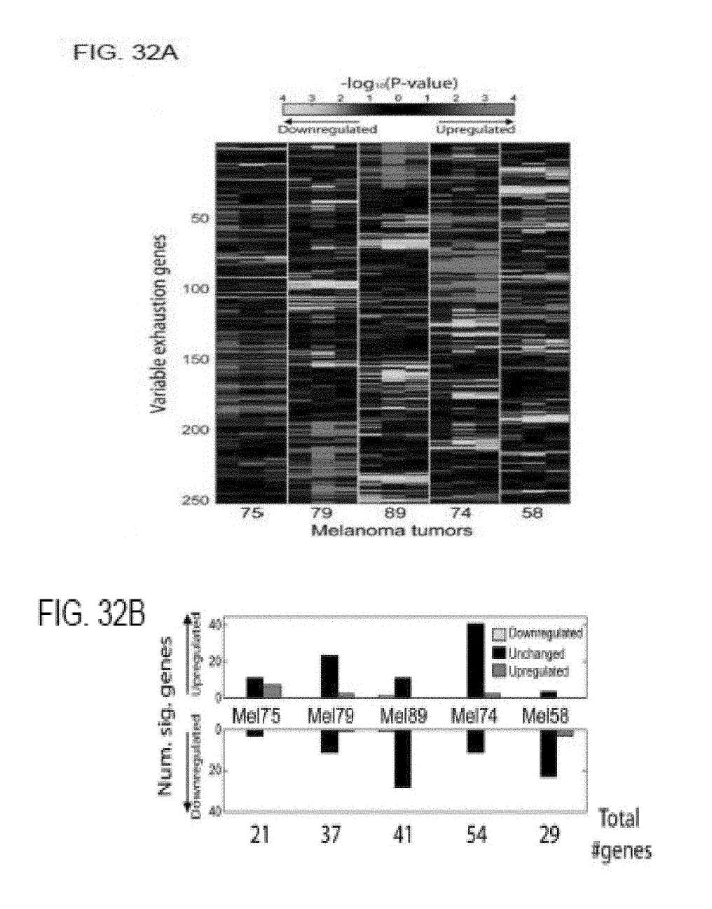

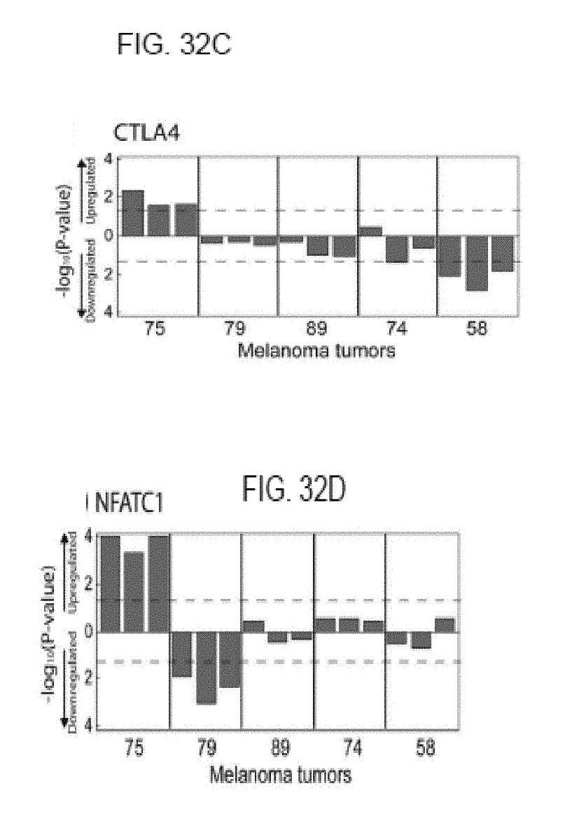

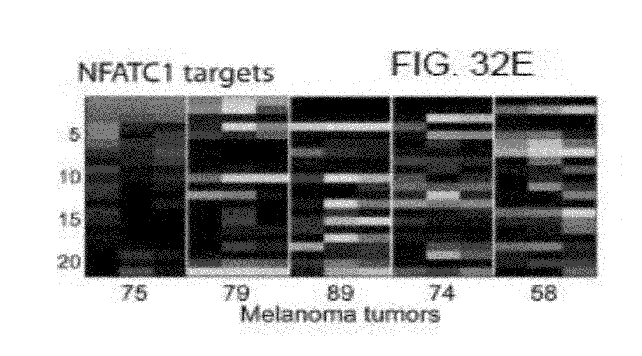

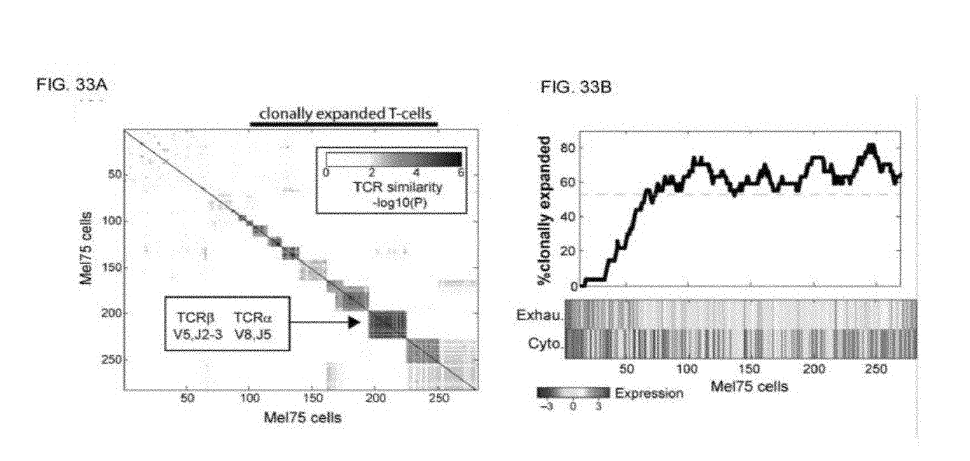

Applicants observed substantial variation between patients in the high exhausted cells, which may mirror the variation in treatment responses or history. Nonetheless, our core exhaustion signature yielded 28 genes that were consistently upregulated in high-exhaustion cells of most tumors, including co-inhibitory (TIGIT) and co-stimulatory (TNFRSF9/4-1BB, CD27) receptors (FIG. 5E and Table 14). In addition, most genes that were significantly upregulated in high-exhaustion cells of at least one tumor had distinct associations with exhaustion across the different tumors (FIG. 5F, 272 of 300 genes with P<0.001 by permutation test; FIG. S22A-B and Table 14). These tumor-specific signatures included variable expression of known exhaustion markers (Table 13), and could be linked to response to immunotherapies or reflect the effects of previous treatments. For example, CTLA4 was highly upregulated in exhausted cells of Mel75 and weakly upregulated in three other tumors, but was completely decoupled from exhaustion in Mel58. Interestingly, Mel58 was derived from a patient with initial response and subsequent development of resistance to CTLA-4 blockade with ipilimumab (FIG. 5F, FIG. 32C). Another variable gene of interest was the transcription factor NFATC1, which was previously implicated in T cell exhaustion (47). NFATC1 and its target genes were strongly associated with the activation-independent exhaustion phenotype in Mel75 (FIG. 32D-E), suggesting a potential role of NFATC1 in the underlying variability of exhaustion programs among patients.

TABLE 14
Exhaustion program genes, related to FIG. 5E/F
Exhuastion-associated genes are listed in the first column in the order that they appear in the heatmaps in
FIG. 5E (top list), and FIG. 5F (bottom list)
Additional 30 columns contain the expression log-ratios (column B through P) and the associated p-values
(columns R through AF) for comparison of high vs. low exhaustion cells in each of the five tumors, each with
three alternative gene-sets to score cells for exhaustion.
P-values were estimated by 10,000 permutations, only for cases with at least two-fold upregulation by one of the
three gene-sets; therefore zeros indicate P <= 10\hat{ }(−4) and NaNs indicate missing non-significant values.
The last 15 columns (columns AH through AV) contain P-values from comparison of exhaustion upregulation in
each tumor to a combination of cells from all other tumors. Sign indicates whether the gene is more or less
upregulated in the specific tumor (i.e. 0.05 correspond to a gene that is more upregulated in a partcular tumor,
while −0.05 correspond to a gene that is less upregulated in a partciular tumor, with p = 0.05 based on 10,000 permutations)

| | Expression log2-ratio from comparison of high vs. low exhaustion cells in each tumor | | | | | | | |
|---|---|---|---|---|---|---|---|---|
| | mel75 expression | | | mel79 expression | | | mel89 expression | |
| | log-ratio | | | log-ratio | | | log-ratio | |
| | | | tumor/ | | | tumor/ | | |
| Gene | Mel75 | viral | circulation | Mel75 | viral | circulation | Mel75 | viral |
| Names | program | (Wherry) | (Baitch) | program | (Wherry) | (Baitch) | program | (Wherry) |
| Consistent | | | | | | | | |
| across tumors | | | | | | | | |
| (FIG. 5E) | | | | | | | | |
| CXCL13 | 3.312930684 | 2.074262977 | 2.947523488 | 1.902343 | 1.533382 | 2.324908 | 5.163968 | 3.967707 |
| TNFRSF1B | 2.999461867 | 1.816699977 | 2.444215257 | 3.100256 | 3.038269 | 2.967502 | 2.396469 | 1.709586 |
| RGS2 | 3.872164337 | 2.727403283 | 3.579022471 | 1.949493 | 0.934253 | 0.812554 | 1.224387 | 2.071313 |
| TIGIT | 3.067236204 | 2.435284642 | 2.241673974 | 2.048262 | 1.936432 | 2.12327 | 0.778375 | 1.525617 |
| CD27 | 3.056197245 | 1.893041958 | 2.543041365 | 1.016713 | 0.308833 | 0.287426 | 0.210744 | 0.33319 |
| TNFRSF9 | 2.893983506 | 2.324879503 | 2.588876346 | 2.102371 | 1.414281 | 1.114887 | 0.142897 | −0.00992 |
| SLA | 2.569832702 | 1.838164585 | 2.057050312 | 2.764392 | 1.834447 | 2.00188 | 2.504309 | 1.621437 |
| RNF19A | 2.96135097 | 2.761526357 | 2.65157748 | 1.718117 | 0.852862 | 1.17018 | 0.941933 | 0.535392 |
| INPP5F | 2.173783159 | 2.005891621 | 2.011528671 | 1.203769 | 1.306366 | 1.276634 | 0.98959 | 0.507219 |
| XCL2 | 1.235512648 | 0.825456292 | 0.944792874 | 1.281504 | 1.876185 | 2.295258 | 0.904837 | 1.749066 |
| HLA- | 1.845491325 | 0.871887038 | 1.377781549 | 1.183536 | 1.459452 | 0.584136 | 0.425308 | 0.287876 |
| DMA | | | | | | | | |
| FAM3C | 1.562400302 | 1.444865168 | 1.40756732 | 1.772647 | 1.557671 | 0.975543 | 0.338394 | 0.266142 |
| UQCRC1 | 0.469003951 | 0.345269963 | 0.354783467 | 1.114473 | 2.222364 | 1.641824 | 0.47412 | 1.824849 |
| WARS | 1.65305276 | 1.190869514 | 1.451220325 | 1.92366 | 2.028299 | 1.955001 | −0.5816 | −0.31752 |
| EIF3L | 0.853804228 | 1.060819549 | 1.109706926 | 0.128691 | 1.081313 | 0.372077 | 0.025211 | 2.437707 |
| KCNK5 | 1.401690446 | 0.898050717 | 0.80841985 | 0.242973 | 0.484445 | 0.778694 | 1.22578 | 0.926468 |
| TMBIM6 | 1.449068162 | 0.555411739 | 1.0778832 | 1.919289 | 1.389959 | 1.997415 | 1.095294 | 0.794735 |
| CD200 | 2.080491281 | 1.424668198 | 1.255597416 | 1.627321 | 0.846131 | 0.998961 | −0.07003 | −0.19943 |
| ZC3H7A | 1.800746214 | 1.513906966 | 1.313198459 | 0.812254 | 0.4393 | 0.467088 | 0.390496 | 0.280661 |
| SH2D1A | 1.337511112 | 0.915806219 | 0.819866056 | −0.31511 | 0.275565 | 0.206968 | 1.410156 | 2.515866 |
| ATP1B3 | 1.055311363 | 0.629682188 | 0.977539251 | −0.10177 | 1.043655 | 0.692404 | −0.33363 | 0.053089 |
| MYO7A | 0.093625152 | 0.085079604 | 0.343623848 | 0.473949 | −0.49134 | 0.401816 | 1.341331 | 1.526063 |
| THADA | 1.690665225 | 1.201138454 | 1.399513494 | 0.905088 | 0.79978 | 0.817263 | 0.673263 | 1.074633 |
| PARK7 | 1.405601753 | 1.886830702 | 1.766259076 | 0.014328 | 1.611124 | 0.658845 | 0.461291 | 1.57344 |
| EGR2 | 1.065864255 | 0.824627041 | 0.834802763 | 0.568467 | 1.036682 | 0.637528 | −0.66789 | −0.7313 |
| FDFT1 | 1.187783332 | 1.031857871 | 1.066466992 | 0.324997 | 0.886523 | 1.005787 | −0.1555 | −0.07995 |
| CRTAM | 1.090748991 | 0.584046588 | 1.242108077 | 0.760366 | 1.150953 | 1.61662 | 0.244529 | 0.002515 |
| IFI16 | 1.340362395 | 0.976428181 | 0.908488721 | 0.114541 | −0.73751 | 0.006688 | 1.547676 | 1.371395 |
| variable | | | | | | | | |
| across | | | | | | | | |
| tumors | | | | | | | | |
| (FIG. 5F) | | | | | | | | |
| GMNN | 0.043027574 | 0.171842265 | 0.144779127 | −0.16897 | 0.152684 | −0.01374 | −0.13428 | 1.501233 |
| AFG3L1P | −0.071151183 | 0.077919663 | 0.044546237 | 0.202711 | −0.29519 | 0.059354 | 0.59596 | 1.224622 |
| CSRP1 | −0.129469728 | −0.081390393 | −0.433407203 | −0.93841 | −1.44046 | −0.0145 | 0.807921 | 1.696062 |
| RBM5 | −0.062501471 | 0.439979335 | 0.196351348 | 0.705714 | −0.42573 | 0.286577 | 1.798667 | 2.449026 |
| AP1M1 | −0.166296903 | −0.768335943 | −0.6720713 | 0.007211 | −0.17268 | −0.08676 | 1.691288 | 2.776494 |
| NUCB2 | 0.881556972 | 0.239736036 | 0.095964286 | 0.348022 | 0.814255 | 0.398238 | 1.533075 | 1.958116 |
| NOP10 | 0.149683203 | 0.542782041 | 0.03901034 | −0.1062 | 0.224016 | −0.13205 | 0.774895 | 2.59629 |
| GFM1 | 0.286809367 | 0.325745216 | 0.4349105989 | 0.42456 | 0.190444 | 0.540836 | 0.565833 | 1.497705 |
| DHRS7 | 0.138738644 | 0.258728751 | 0.095937832 | 0.581986 | −0.322 | 0.185116 | 1.254592 | 2.228929 |
| SSU72 | 0.45241041 | 0.383321038 | 0.294432984 | −0.52079 | −0.48351 | −0.1727 | 1.817829 | 2.201066 |
| SBDS | 0.094145363 | −0.091460228 | −0.090246662 | −0.12381 | 0.327272 | −0.27703 | 0.869645 | 1.580922 |
| ATP6V1B2 | 0.612364922 | 0.519739479 | 0.407802079 | 0.098141 | 0.769531 | 0.931401 | 0.395432 | 1.332202 |
| VAPA | 0.592418734 | 0.017830025 | 0.317382438 | 0.453913 | 0.964504 | 0.947221 | 1.289721 | 1.66887 |
| CSNK2A1 | 0.333499146 | 0.576268847 | 0.378711978 | 0.314716 | 0.64711 | 0.454751 | 0.507651 | 1.4731 |
| LINC00339 | 0.000787099 | −0.005790472 | 0.126938699 | 0.382733 | 0.319703 | 0.097808 | 0.488001 | 1.206209 |
| MRPL4 | −0.05291909 | −0.248341777 | −0.325456543 | 0.954438 | 0.968095 | 0.433131 | 0.714926 | 1.591578 |
| PPP1R2 | 0.708248895 | −0.416790518 | 0.51829621 | 0.637519 | 1.650811 | 1.682616 | 1.257319 | 1.555086 |
| SMG1 | 0.24014141 | −0.220559107 | −0.093885207 | 0.92039 | 0.686976 | 0.776151 | 0.768321 | 1.258011 |
| OIP5- | −0.421250676 | 0.054146426 | −0.306721213 | 0.745885 | 0.998988 | 1.10055 | 0.894821 | 1.150241 |
| AS1 | | | | | | | | |
| LPAR2 | −0.275312361 | −0.37744524 | −0.323147451 | −0.34247 | −0.05257 | −0.05544 | 0.240118 | 1.445894 |
| LSMD1 | −0.062045249 | −0.085331468 | −0.156453881 | 0.201232 | 0.191145 | −0.00192 | 1.31328 | 1.504531 |
| STAG3L4 | 0.208189665 | 0.294570142 | 0.329089633 | 0.195268 | 0.320167 | 0.121198 | 0.953212 | 1.394307 |
| P4HB | −0.102174268 | 0.650942668 | −0.080884203 | 0.025826 | 0.549314 | 0.399946 | 0.799846 | 2.419971 |
| SKP1 | 0.645024799 | 0.436055679 | 0.413397937 | 0.800583 | 0.525398 | 0.823875 | 1.845179 | 2.279553 |
| PTBP1 | 0.283339082 | 0.217413126 | 0.551373639 | 0.241898 | 0.608438 | 0.632225 | 1.320938 | 1.998618 |
| TSTA3 | −0.32366765 | 0.013884689 | −0.313551022 | 0.252992 | 0.107914 | 0.470213 | 1.528378 | 1.849042 |
| TBCB | −0.6846733 | −0.1501031 | −0.440431014 | −0.79423 | 0.206511 | 0.063237 | 1.332235 | 2.29277 |
| SMC5 | −0.087783445 | −0.55180393 | −0.55884345 | −0.37557 | 0.535856 | −0.19135 | 1.071783 | 1.447682 |
| KLHDC2 | 0.395429469 | 0.668556916 | 0.371742474 | −0.10317 | 0.159295 | −0.06556 | 0.464198 | 1.582696 |
| MPV17 | 0.116599787 | 0.209519428 | 0.004839974 | 0.337336 | −0.18099 | −0.33336 | 1.607473 | 2.539661 |
| RBPJ | 0.428501515 | 0.25076715 | 0.438313819 | 0.052933 | −0.10794 | −0.04362 | 1.500064 | 2.190548 |
| POP5 | 0.737424053 | 0.551295498 | 0.601295499 | 0.551109 | 0.23027 | −0.05578 | 0.670319 | 1.523417 |
| PPAPDC1B | 0.456002002 | 0.552300346 | 0.702249897 | 0.431746 | −0.91909 | −0.175 | 0.791801 | 1.245959 |
| IMP3 | 0.868673963 | 0.640438295 | 0.90397918 | −0.07056 | −0.05648 | 0.493301 | 0.698 | 2.090965 |
| RNPS1 | 1.32274794 | 1.06910008 | 0.997867484 | 0.845931 | 1.172472 | 1.1871 | 0.37896 | 0.940734 |
| NFE2L2 | 0.315270113 | 0.345583993 | 0.461493517 | 0.650763 | 1.315303 | 0.877157 | −0.24908 | 0.304611 |
| SOD1 | 1.115550531 | 0.595670174 | 0.765317509 | 1.108039 | 1.924778 | 1.841061 | 0.702326 | 0.962281 |
| CD8B | 1.386005909 | 0.601382631 | 1.128332385 | 0.656311 | 0.56138 | 0.631017 | 1.057392 | 0.672517 |
| PTPN6 | 1.532873235 | 1.059501171 | 1.272809186 | 1.283707 | 0.723197 | 1.16716 | 1.593161 | 1.221782 |
| HSPA1B | 2.011326357 | −0.017272685 | 0.482033079 | 1.355479 | −0.70202 | 0.061356 | 1.333737 | 1.19193 |
| CD2BP2 | 1.025380603 | 1.107130771 | 1.13179342 | 0.972474 | 0.313994 | 1.300398 | 0.78121 | 1.096756 |
| ALDOA | 1.313853281 | 0.885911011 | 1.170827822 | 0.183503 | 0.132005 | 0.603886 | 0.863351 | 0.407278 |
| ZFP36L1 | 1.377932802 | 1.046667112 | 1.011990109 | 1.287774 | 0.522387 | 0.640023 | 1.668201 | 0.212425 |
| HSPB1 | 1.998780423 | 1.499266873 | 2.010969362 | 0.88779 | −0.91591 | −0.76742 | 1.20899 | 0.882177 |
| HSPA6 | 1.35903358 | 0.503171112 | 0.87333988 | −0.19713 | −1.68588 | −1.30244 | 0.13999 | 0.447783 |
| ARHGEF1 | 1.126546499 | 0.515397194 | 0.820448612 | 0.131261 | −0.3879 | 0.080298 | 0.038873 | 0.297429 |
| LUC7L3 | 1.447736541 | 1.519295485 | 1.175206442 | −0.30414 | −0.0943 | 0.068382 | 0.582282 | 1.336517 |
| GPR174 | 1.293313484 | 1.1973819 | 1.320879739 | −1.1577 | −1.90851 | −0.89633 | −0.61215 | 0.387192 |
| ENTPD1 | 1.038604866 | 1.188716869 | 0.80969983 | 0.010669 | −0.18254 | −0.22731 | 0.421867 | 0.245372 |
| RASSF5 | 1.782804631 | 1.596770053 | 1.615600953 | 0.332968 | 0.021103 | 1.118996 | 0.612163 | 0.995728 |
| IPCEF1 | 1.167524116 | 0.822654381 | 0.863026784 | 0.251741 | 0.071486 | 0.235929 | 0.554976 | 0.477973 |
| ARNT | 1.381979732 | 0.459696916 | 1.024572842 | −0.3619 | −0.12367 | −0.06941 | 0.260473 | 0.288848 |
| NAB1 | 1.534472803 | 1.14759428 | 1.124383759 | 0.682645 | 0.028969 | 0.582381 | 0.297359 | 0.453696 |
| APLP2 | 1.034902448 | 0.34573962 | 0.562004519 | 0.604263 | 0.218654 | 0.434346 | −0.3392 | −0.58647 |
| PRKCH | 2.095651028 | 1.250367974 | 1.383961633 | 0.973384 | 0.334772 | 1.547998 | 0.546222 | −0.09044 |
| SEMA4A | 1.27878448 | 0.670815166 | 0.908162097 | 0.589 | 0.586758 | 0.22612 | −0.07217 | −0.00775 |
| PPP1CC | 1.237735482 | 1.239916799 | 1.496451835 | 0.350992 | 0.398534 | 0.304209 | −0.81761 | −0.79027 |
| LAG3 | 1.469524443 | 0.808447296 | 1.193318776 | 0.552084 | 0.343635 | 0.563471 | 0.377612 | 0.306796 |
| HSPA1A | 2.183724617 | −0.052501429 | 0.905684708 | 1.412451 | −0.98904 | 0.023958 | 0.451536 | −0.13048 |
| SNAP47 | 1.996664962 | 1.521180094 | 1.789974077 | 1.646128 | 0.773017 | 0.831949 | 1.768177 | 0.311002 |
| CCL4L2 | 1.518782661 | 1.621224804 | 1.656527601 | 1.659094 | 0.720119 | 0.986238 | 0.773504 | −0.40149 |
| ARID4B | 1.555979452 | 1.212190586 | 1.524628823 | 1.181436 | 0.389736 | 0.736853 | 0.952166 | 0.25604 |
| LYST | 2.230049736 | 1.241313793 | 1.574512297 | 0.763879 | −0.12037 | 0.547757 | 0.662939 | −0.44328 |
| NMB | 1.678455804 | 0.921489719 | 0.73858918 | 0.435093 | 0.760483 | 0.481887 | 0.365894 | 0.099393 |
| LIMS1 | 1.474286378 | 0.956750271 | 0.95305825 | 0.628188 | 0.862224 | 0.385559 | 0.778963 | 0.556935 |
| ITK | 1.414179216 | 1.43890658 | 1.478553088 | 0.483651 | 0.683414 | 0.303191 | 0.107844 | 0.390596 |
| RILPL2 | 0.959915326 | 1.135058344 | 1.293258504 | 0.462018 | 0.466823 | 0.831535 | 0.071116 | 0.553541 |
| RGS3 | 1.154584995 | 1.15319424 | 1.467784987 | 0.4524 | 0.744248 | 0.491837 | 0.164149 | 0.279297 |
| TRAT1 | 2.048157243 | 1.778604554 | 1.184359317 | −0.18319 | 1.056644 | 0.61408 | −0.6924 | −0.30911 |
| ELF1 | 1.135502002 | 0.744026603 | 0.705549723 | −0.09728 | −0.03617 | −0.30845 | −0.15139 | −0.70932 |
| OSBPL3 | 1.244493756 | 0.754910428 | 0.958328332 | 0.546178 | 0.622905 | 0.490143 | 0.264703 | 0.34498 |
| BIRC3 | 1.193199089 | 0.457161488 | 0.85206847 | 0.282324 | 0.357573 | 0.224471 | 0.004753 | −0.36429 |
| PTGER4 | 1.311750447 | 1.168490662 | 1.135332759 | 0.341347 | 0.466851 | −0.03023 | −0.81506 | −1.13053 |
| SERINC3 | 1.453349403 | 1.19830239 | 1.078429788 | 0.877679 | 1.657278 | 0.986886 | −0.91209 | −2.07222 |
| MED7 | 0.657265457 | 0.854446675 | 0.687406073 | 0.526406 | 0.436239 | 0.751896 | 0.019976 | −0.28637 |
| DDX3X | 1.29061396 | 0.757199323 | 1.036371277 | 0.824003 | 0.265828 | 0.120602 | −0.13795 | −0.26647 |
| THEM6 | 0.042372464 | 0.440844807 | 0.436704995 | 0.229215 | −0.19033 | 0.213359 | −0.3112 | −0.38826 |
| P4HA1 | 0.538676008 | 0.119204379 | 0.334805341 | 0.272292 | 0.207532 | 0.47147 | 0.396575 | −0.56505 |
| HIBCH | 0.340376043 | 0.327380101 | 0.151034238 | −0.0236 | −0.65387 | −0.43821 | −0.59167 | −0.891 |
| VCAM1 | 1.64009384 | 0.579518782 | 1.236128356 | 1.181157 | −0.21288 | 0.275033 | 0.710478 | −0.78889 |
| FABP5 | 1.612342328 | 0.712514671 | 1.315489417 | 0.443385 | 1.115881 | 1.004397 | 1.213656 | 0.861389 |
| NOL7 | 0.277805876 | 0.024089054 | −0.047835004 | 0.655765 | 1.077861 | 1.316975 | −0.00132 | −0.05043 |
| SEC14L1 | 0.081430686 | −0.129754372 | 0.108992586 | 0.627199 | 0.57787 | 1.062197 | 0.491738 | 0.519502 |
| UBA2 | −0.092226466 | 0.24700281 | 0.154951634 | 0.280709 | 0.808909 | 0.970645 | 0.304066 | 0.530309 |
| CDCA4 | −0.126508543 | 0.128689169 | 0.180970828 | 0.12064 | 1.005329 | 0.399137 | 0.542623 | 0.656551 |
| ATP5I | −0.327298329 | −0.349050236 | −0.920455232 | 0.155432 | 0.67103 | −0.02518 | 0.814725 | 1.066032 |
| ALKBH3 | −0.188196002 | −0.111949186 | −0.41617222 | −0.02238 | −0.03832 | −0.26114 | 0.185297 | 0.234059 |
| DND1 | −0.060119977 | 0.032905932 | −0.262716371 | 0.121023 | −0.05467 | −0.07239 | 0.723372 | 0.112781 |
| RNF185 | −0.089462381 | 0.019416524 | −0.393030332 | −0.30534 | −0.24945 | −0.11258 | 0.538053 | 0.262645 |
| AFAP1L2 | 0.152547874 | −0.318203746 | −0.211110775 | 0.262559 | 0.281342 | 0.50659 | 0.567692 | 0.336931 |
| GLOD4 | 0.358009428 | 0.107375551 | 0.018136102 | 0.676799 | 1.052775 | 0.609451 | 0.734409 | 0.69556 |
| PIP5K1A | −0.292406001 | −0.133590617 | −0.003760948 | −0.00051 | 0.485555 | 0.291551 | −0.08627 | 0.311723 |
| ATF4 | 0.085708928 | −0.084593497 | 0.760824626 | 0.392588 | 0.588179 | 0.553381 | 0.394735 | 1.509907 |
| PIGO | 0.298036607 | 0.006383643 | 0.167832861 | 0.33748 | 0.102584 | 0.153793 | −0.09786 | −0.02296 |
| OPA1 | 0.154143981 | 0.14808268 | 0.275399824 | 0.154064 | 0.388671 | −0.07498 | −0.14784 | −0.08245 |
| CCT3 | 0.497652111 | 0.448074493 | −0.106468226 | 0.213517 | 0.200512 | −0.36047 | −0.09796 | 0.320487 |
| EXOSC6 | −0.271473 | −0.377455003 | −0.325228666 | −0.13313 | −0.70128 | −0.29486 | −0.21506 | −0.03847 |
| KIAA1429 | 0.035542179 | −0.143608507 | 0.176855427 | −0.32497 | −0.0122 | 0.078369 | 0.090747 | 0.162601 |
| NDFIP2 | 1.000529124 | 0.713573212 | 0.916957154 | 0.269833 | 0.453574 | 0.914847 | 0.185453 | 0.708124 |
| TMEM222 | 0.01927459 | 0.059991453 | 0.432444724 | −0.13321 | 0.061383 | 0.157072 | −0.37596 | 0.512837 |
| MYO1G | −0.021541261 | 0.354336769 | −0.090091368 | −0.79222 | −0.12867 | −0.31777 | 0.444614 | 0.213143 |
| LBR | −0.330259621 | −0.437386804 | −0.653002557 | −0.14327 | 0.329927 | 0.787167 | 0.398744 | 0.651779 |
| EXT2 | 0.375137992 | 0.060183838 | 0.307469179 | −0.03194 | 0.214041 | 0.793301 | 0.186481 | 0.602669 |
| SARDH | 0.780291764 | 0.655891551 | 0.71980072 | 0.298395 | 0.060619 | 0.614921 | 1.001938 | 0.709208 |
| POLR2I | 0.411361291 | 0.466883266 | 0.424819576 | −0.61892 | −0.54023 | −0.92447 | 0.17054 | 0.305786 |
| HNRNPD | 0.583688852 | 0.486005257 | 0.845653113 | −0.23169 | −0.65989 | −0.24164 | 0.854117 | 1.518836 |
| NAAA | 0.171373703 | −0.266902261 | −0.079535995 | −0.32806 | −1.36017 | −0.7265 | −0.28776 | −0.28367 |
| ARID5A | 0.717283712 | 0.135137524 | 0.893579557 | −0.6991 | −0.93753 | −0.63467 | 0.63719 | 0.120282 |
| PDRG1 | −0.257798832 | −0.188927412 | −0.405771825 | −0.65658 | −1.12104 | −0.95987 | 0.252316 | 0.324749 |
| BCAP31 | 0.248712094 | 0.039964586 | 0.411051754 | −0.38116 | −0.30212 | −1.44155 | 1.149001 | 2.046816 |
| UQCRFS1 | 0.244003342 | 0.627992936 | 0.745441734 | −0.44459 | 0.390037 | 0.185785 | 1.107422 | 1.946439 |
| SNRNP40 | 0.136098914 | −0.223312038 | 0.020633916 | −0.02307 | −0.1459 | −0.36661 | 0.210973 | 0.866088 |
| ASB8 | −0.108745262 | −0.269424784 | −0.154395572 | 0.381666 | 0.209538 | 0.303254 | −0.13418 | 0.380815 |
| MRPL52 | −0.084064212 | 0.115934757 | 0.065004735 | 0.208567 | 0.153299 | −0.33161 | −0.04853 | −0.00273 |
| TUG1 | 0.437698058 | 0.581478939 | 0.460903566 | −0.0966 | 0.404788 | 0.550919 | 0.228966 | 0.502488 |
| CCND2 | 0.271370405 | 0.60236512 | 0.688135369 | 0.258937 | 0.388129 | 0.33637 | 0.473715 | 0.915072 |
| NAA20 | −0.199732482 | 0.034489683 | −0.253097065 | −0.76689 | −0.99558 | −0.25126 | 0.03323 | 0.108347 |
| HLA- | 0.718032093 | 0.145829492 | 0.432274133 | −0.2936 | −0.12783 | 0.125854 | 2.086487 | 0.695833 |
| DPA1 | | | | | | | | |
| TOX | 1.763680529 | 0.811412812 | 1.230711584 | 0.477088 | −0.04763 | 0.541605 | 1.27303 | 0.506332 |
| TMEM205 | 0.262817719 | 0.234402817 | 0.666366803 | −0.18657 | −0.40025 | −0.08797 | −0.62806 | 0.032414 |
| TPI1 | 1.590740398 | 0.588366329 | 1.586290469 | −0.25626 | 0.033923 | 0.554393 | 0.47471 | 1.364495 |
| HADHA | 1.201943538 | 1.247942158 | 0.928195512 | −1.55492 | −0.73625 | −0.77661 | −0.0692 | 0.347628 |
| STAT3 | 1.361211716 | 0.747990389 | 0.948730745 | 0.621704 | −0.07355 | 0.425333 | 0.594964 | 1.044868 |
| GMDS | 1.095785438 | 0.696650797 | 0.715566479 | −0.04052 | −0.65174 | −1.0781 | 0.246792 | 0.125568 |
| SIRPG | 1.376454997 | 0.665418641 | 1.412637128 | −1.00957 | −0.30489 | 0.568758 | 1.064944 | 0.134373 |
| ITM2A | 2.977499864 | 1.895044787 | 2.193733749 | 0.178731 | 0.320537 | 1.36315 | 1.335396 | 1.864763 |
| TBC1D4 | 1.608100031 | 0.821968022 | 1.207923504 | 0.179446 | 0.293976 | 0.109901 | 0.476205 | 0.676084 |
| HNRNPM | 1.413649588 | 0.831555256 | 1.525972231 | −1.05907 | −0.61527 | 0.265439 | −0.51384 | −0.37695 |
| ASB2 | 1.251207504 | 1.002848378 | 0.943960897 | 0.607734 | 0.771546 | 1.043851 | 0.263048 | 0.996108 |
| IGFLR1 | 2.616319498 | 1.068099693 | 2.098449556 | 1.758737 | 0.718694 | 1.001359 | 0.581381 | 0.57966 |
| CD2 | 1.150444265 | 0.433439232 | 0.362947257 | 0.782524 | −0.09669 | 0.111907 | −0.48779 | 0.080094 |
| COTL1 | 0.515720837 | 0.198501381 | 0.108658672 | −0.83532 | −1.40503 | 0.168078 | −0.76977 | −0.42037 |
| PBRM1 | 0.008620138 | 0.006590668 | −0.022029041 | 0.17964 | 0.108284 | 0.429848 | 0.075208 | 0.344958 |
| DUT | 0.399540121 | 0.65015255 | 0.585679832 | 0.594714 | 1.351903 | 1.057338 | 0.544438 | 1.066196 |
| LMF2 | 0.307389613 | 0.166784087 | −0.051994037 | 1.097763 | 1.393787 | 1.015263 | 0.830791 | 1.170537 |
| TAF15 | 0.249141204 | 0.445364705 | 0.118349038 | 0.816265 | 1.387048 | 0.811674 | 0.575234 | 0.710003 |
| H2AFY | 0.307752209 | 0.1521224 | 0.657823706 | 0.327934 | 1.14378 | 0.674421 | 0.343279 | 1.536905 |
| CEP57 | 0.876575938 | 0.542127567 | 1.04377651 | 0.588249 | 0.712554 | 0.624363 | 0.765157 | 1.134142 |
| AMDHD2 | −0.051735663 | 0.00190803 | 0.294956325 | 0.432604 | −0.03546 | 0.112963 | 0.488117 | 0.202342 |
| SERINC1 | 1.129247864 | 0.53200722 | 0.531081425 | 0.392857 | 0.513207 | 0.206157 | 0.970832 | 0.847416 |
| CKS2 | 1.072847758 | 0.357162351 | 0.90914841 | 0.865621 | 0.219878 | 0.511007 | 0.75919 | 1.437176 |
| PTPN11 | 1.319498007 | 1.207932377 | 1.197385011 | 0.966305 | 0.31961 | 0.541434 | 0.788892 | 1.206738 |
| DDX3Y | 1.183233711 | 1.291140673 | 1.119592424 | 0.1921 | 0.054788 | 0.027078 | 0.383849 | 0.680354 |
| IRF9 | 1.878616017 | 1.086375279 | 1.512377275 | 0.447343 | −0.15 | 0.130645 | 1.58042 | 1.48688 |
| FYN | 1.444041407 | 1.018597447 | 1.104055507 | −0.48429 | −0.47609 | −0.02893 | 1.224496 | 1.016933 |
| HSPD1 | 1.208198663 | 1.05372992 | 1.337071169 | 0.838551 | 0.404839 | 1.408919 | 0.715702 | 1.084178 |
| FPGS | 1.355547156 | 1.188630161 | 0.98953347 | 0.485478 | 0.259058 | 0.892308 | −0.50871 | 1.184799 |
| CCT2 | 1.08253103 | 0.75304456 | 0.943100019 | 0.446365 | 0.42508 | 0.750063 | −0.21371 | 0.839192 |
| GNAS | 1.179063025 | 1.131070538 | 1.251606246 | 1.03141 | 1.424083 | 1.697233 | 0.900231 | 0.871713 |
| FAIM3 | 2.426863138 | 1.206279614 | 1.706168485 | 0.934786 | 0.938966 | 0.648895 | 0.685702 | 0.051713 |
| ETV1 | 1.406785311 | 0.991489528 | 1.141312005 | 0.674663 | 0.70102 | 0.567019 | 1.215873 | 0.786232 |
| BCL6 | 1.025700596 | 0.507071558 | 0.703993079 | 0.441169 | 0.303076 | 0.396493 | 0.516164 | 0.649468 |
| SLC38A1 | 1.322457119 | 1.267927568 | 1.462238314 | 0.557253 | −0.21456 | 0.361313 | 0.439357 | 0.750614 |
| PDE7B | 1.669299269 | 1.197372004 | 1.275856398 | 0.816225 | 0.034747 | 0.464835 | 0.745068 | 0.219427 |
| STAT1 | 1.288531473 | 1.224716916 | 1.202852623 | 0.222985 | −1.38484 | −0.34013 | 0.691912 | −0.57695 |
| EIF3H | 1.435879952 | 0.866502474 | 1.017699196 | −0.13228 | 0.282104 | 0.130292 | 0.820465 | 0.726501 |
| EID1 | 2.219389373 | 1.566207301 | 2.07401064 | 0.023779 | 0.233941 | −0.00422 | 1.891068 | 1.499255 |
| ID3 | 2.156181502 | 1.874951827 | 2.194440091 | −0.24615 | −0.42221 | 0.348782 | 0.650554 | 0.939375 |
| PSAP | 1.482493642 | 1.251714914 | 1.583987777 | −0.16672 | −1.09955 | 0.214896 | 0.91274 | 0.883965 |
| DPP7 | 1.286780009 | 1.14990123 | 0.819394139 | 0.061798 | −0.28247 | 0.746249 | 0.976358 | 1.440867 |
| PJA2 | 1.135010415 | 1.072482681 | 1.193836484 | 0.317889 | 0.273972 | −0.17391 | 0.910362 | 1.80749 |
| TARDBP | 1.085987462 | 1.307037121 | 0.917550551 | −0.40006 | −0.85037 | −0.1677 | −0.33295 | 1.041841 |
| SRSF1 | 0.956369952 | 0.333782486 | 1.080567001 | 0.155516 | 0.429937 | 0.421241 | 0.5436 | 0.578719 |
| GABPB1 | 0.895910769 | 0.727766526 | 1.070519023 | −0.19441 | 0.295627 | −0.03526 | 0.167344 | −0.19402 |
| RGS4 | 2.098079303 | 1.373799718 | 1.566364058 | 0.54745 | −0.11883 | −0.22131 | 0.378318 | −2.22E−16 |
| SPTAN1 | 1.203063542 | 0.728124694 | 0.848187751 | 0.08366 | −0.45946 | −0.17811 | −0.19259 | −0.4073 |
| NFATC1 | 1.848389397 | 1.535430539 | 1.636742466 | 0.284929 | −0.01452 | 0.158721 | 0.717018 | 0.437112 |
| HAVCR2 | 1.829069166 | 1.556593935 | 1.930021168 | 0.099598 | −0.60911 | −0.61977 | 0.242262 | −1.08348 |
| PDCD1 | 3.669342943 | 2.588502543 | 2.199613903 | 1.069568 | −0.40108 | 0.391635 | 0.082365 | −0.74739 |
| SRSF4 | 1.282668848 | 0.584600779 | 0.846924585 | −0.35889 | −0.85482 | −0.60332 | −0.54135 | −0.59792 |
| GFOD1 | 1.435124282 | 0.805969237 | 1.361869686 | 0.960744 | 0.593105 | 0.118554 | −0.11539 | 0.205735 |
| MRPS21 | 1.484504799 | 0.887231467 | 1.129799967 | −0.21745 | 0.800712 | 0.12531 | −0.16722 | −1.15734 |
| AP3S1 | 1.107940879 | 1.581832944 | 1.253456392 | 0.169254 | −0.10696 | 0.471832 | −0.2926 | −0.99593 |
| GPBP1 | 1.148850889 | 0.769667726 | 0.925121393 | 0.259536 | 0.289562 | 0.401878 | −0.65131 | −1.99319 |
| BTLA | 1.271430365 | 0.858356192 | 1.248515815 | 0.636222 | 0.522199 | 1.194408 | −0.41954 | −0.71038 |
| PAM | 1.73788941 | 0.820404499 | 1.049542256 | 0.856856 | 0.856898 | 0.221977 | 0.10668 | −1.12133 |
| CBLB | 1.726964017 | 0.685784278 | 1.348107924 | 1.75033 | 0.7767 | 1.342716 | 0.922017 | −0.31335 |
| ATHL1 | 2.125409979 | 1.363305151 | 1.552296955 | 2.316883 | 1.811821 | 0.70353 | 0.160417 | 0.042384 |
| MGEA5 | 1.452502385 | 1.351892146 | 1.180358714 | 1.808464 | 1.237657 | 1.028661 | 0.293778 | −0.21566 |
| IRF4 | 1.086257706 | 1.026211452 | 1.416294836 | 1.032828 | 1.126156 | 0.941122 | 0.409479 | −0.81235 |
| UBE2F | 1.266533204 | 1.062885597 | 1.424973207 | 0.719937 | 0.76793 | 0.846906 | 0.206919 | −0.24274 |
| SFXN1 | 1.385516086 | 0.939185664 | 1.164065851 | 0.780422 | 0.756912 | 0.472239 | −0.39917 | 0.219162 |
| DGKH | 1.495251313 | 1.059658266 | 1.27309139 | 0.717511 | 0.465334 | 1.035035 | 0.218553 | 0.314905 |
| FCRL3 | 3.728309035 | 2.308838656 | 2.83349104 | 1.768635 | 0.095319 | 0.576272 | 0.497876 | 0.094927 |
| PYHIN1 | 1.25158173 | 0.254226468 | 0.536026843 | 0.158718 | −0.38493 | −0.53301 | −1.02845 | −0.90257 |
| EIF1B | 1.13240743 | 0.650847498 | 0.670678234 | 0.732381 | 0.105974 | 0.035768 | −0.78063 | −0.58514 |
| RAPGEF6 | 1.494465106 | 0.766069045 | 1.016077044 | 1.126921 | 0.221664 | 0.912966 | −0.06064 | 0.046427 |
| SNX9 | 1.577860495 | 0.903569889 | 1.13581723 | 1.825853 | 0.655829 | 0.995588 | 0.469539 | 0.239813 |
| IL6ST | 1.451523879 | 0.940122764 | 1.007296058 | 1.515685 | 0.220471 | 0.502483 | 0.837996 | −0.10132 |
| PTPN7 | 1.636471834 | 1.474950361 | 1.437269995 | 1.339936 | 0.942464 | 1.480821 | 1.285213 | 0.523995 |
| CREM | 1.420381394 | 1.305847845 | 1.409075721 | 0.989237 | 0.891545 | 0.545146 | −0.10716 | −0.25254 |
| HNRPLL | 1.404292848 | 1.251582808 | 1.565093404 | 0.938057 | 0.795733 | 0.656747 | 0.664457 | 0.022873 |
| FUT8 | 1.03026227 | 1.336651812 | 1.143972993 | 0.725937 | 0.823277 | 0.606961 | −0.20924 | −0.49135 |
| LITAF | 1.847970051 | 1.953175486 | 1.371124565 | 1.347181 | 0.942992 | 1.582168 | −0.12376 | −1.41878 |
| TSC22D1 | 1.207694382 | 0.642114119 | 0.910783779 | 1.55472 | 0.531984 | 0.864494 | 0.026654 | 0.033668 |
| TRAF5 | 2.064677952 | 1.013096178 | 1.561245448 | 1.631757 | 1.536782 | 1.477133 | 1.471409 | −0.09583 |
| ATP6V0B | 1.104608059 | 1.221930988 | 0.852783134 | 0.415843 | 1.176887 | 0.426354 | −0.33978 | −0.74055 |
| SRSF6 | 0.95639052 | 0.886470556 | 1.114084242 | 0.440808 | 0.246789 | −0.08663 | −0.62811 | −0.52759 |
| ELMO1 | 1.29100362 | 1.029744167 | 0.77545325 | −0.10433 | 0.546682 | 0.064462 | −0.4352 | −0.49677 |
| IRF8 | 2.154089157 | 2.203381286 | 1.94032725 | 0.675898 | 0.732793 | 0.675711 | 0.237049 | −0.47387 |
| TAGAP | 1.366637121 | 1.104414543 | 1.702679578 | 0.446179 | 0.002969 | −0.33689 | −1.8628 | −2.09086 |
| CADM1 | 2.058821862 | 1.037555958 | 1.51803124 | 0.711456 | 0.856303 | 0.560391 | 0.155395 | 0.323803 |
| SPRY2 | 1.830366904 | 0.993711797 | 0.778009129 | 0.20154 | 0.538912 | 0.7264 | 0.438243 | 0.179165 |
| CTLA4 | 2.112817255 | 1.737924436 | 1.78610526 | 0.940203 | 1.028106 | 0.788211 | 0.950634 | 0.360266 |
| ANKRD10 | 1.277935818 | 0.477360235 | 0.469925642 | −0.20261 | −0.51573 | −0.80763 | 0.259223 | −0.50896 |
| KLRK1 | 1.399918242 | 0.27675044 | 0.425020303 | 0.746794 | −0.7027 | 0.090303 | 0.666111 | −0.58332 |
| TP53INP1 | 1.457196161 | 0.56723504 | 0.945503338 | 1.235214 | 0.314193 | 0.598506 | 0.570793 | 0.200328 |
| NR4A2 | 1.213947033 | 1.076621881 | 1.37928836 | 1.023902 | 0.226256 | 0.183376 | −0.68754 | −0.44247 |
| ZNF292 | 1.112530303 | 1.0144105 | 1.185212929 | 0.539638 | 0.775151 | 0.909212 | 0.202204 | −0.04896 |
| MIF4GD | 0.833450486 | 1.05532766 | 0.97220069 | 0.607011 | 0.871253 | 0.374207 | −0.65757 | 0.032883 |
| ING3 | 0.379629244 | 0.254695319 | 0.437292983 | 0.313605 | 1.611961 | 1.082373 | −0.58336 | −0.56659 |
| SQSTM1 | 0.425304438 | 0.610717845 | 0.988891345 | 1.004001 | 1.819091 | 1.752082 | 0.001683 | 0.583508 |
| CLK4 | 0.54414765 | 0.473878316 | 0.669493227 | 0.601875 | 1.467434 | 0.710695 | −0.61275 | −0.12723 |
| NCBP2 | 0.880835016 | 0.859750323 | 0.851293112 | 0.519318 | 1.703887 | 1.032692 | −0.00925 | −0.36409 |
| SET | 0.451874407 | 0.309925087 | 0.461561847 | 0.226276 | 1.679661 | 0.838753 | −1.04072 | −0.04669 |
| PSME3 | 0.509013732 | 0.475890345 | 0.508850734 | 0.930121 | 1.21954 | 1.029992 | −0.16221 | 0.311306 |
| IQCB1 | 0.013996298 | 0.063996592 | −0.045005139 | 0.871463 | 1.143281 | 0.894981 | −0.53263 | 0.187411 |
| RGCC | 0.24885336 | −0.160773088 | 0.021514853 | 0.927153 | 1.802798 | 1.617178 | 0.073407 | 0.197909 |
| C20orf111 | 0.003974358 | −0.350756044 | −0.288491514 | 0.348763 | 1.178669 | 1.08792 | −0.30875 | −0.23304 |
| MPP1 | 0.140348483 | −0.08432789 | 0.053178719 | 1.339736 | 1.533979 | 1.465085 | −0.11597 | 0.116082 |
| CALR | −0.611269744 | −0.348746679 | −0.274495191 | 1.151985 | 2.266154 | 2.166364 | −1.62938 | −0.61249 |
| TMEM160 | 0.061452285 | −0.329938001 | −0.204337388 | 0.210353 | 1.188301 | 0.720347 | 0.252442 | 0.464559 |
| SRGN | 1.499624442 | 0.586354834 | 0.845680692 | 1.794715 | 1.296434 | 1.343558 | 1.186608 | 0.719717 |
| EWSR1 | 1.228722093 | −0.248805265 | 0.053549813 | 0.986405 | 0.772278 | 1.728623 | 0.639167 | −0.32514 |
| EZR | 1.244755035 | 1.362548779 | 0.711574356 | 1.607795 | 1.511479 | 1.807212 | 0.934776 | 0.253583 |
| FTSJ3 | 0.445305924 | 0.291253949 | 0.486536573 | 0.730165 | 1.129672 | 0.899222 | −0.14907 | −0.03622 |
| LRMP | 1.15879917 | 0.426277911 | 0.616052106 | 0.927016 | 0.913392 | 0.618848 | 0.70693 | 0.391718 |
| GBP2 | 2.797732545 | 2.124022172 | 2.229827191 | 2.01263 | 2.194188 | 1.408159 | 1.871806 | 1.402723 |
| MPG | 1.003694564 | 0.543380296 | 0.393406807 | 0.177206 | 0.73196 | 0.59819 | 0.320896 | 0.563695 |
| RELA | 0.71300163 | 0.712144514 | 0.455301277 | 0.655632 | 1.261523 | 1.016636 | 0.54387 | 0.604358 |
| KLHDC4 | −0.201948143 | 0.207028431 | 0.266778092 | 0.526649 | 1.334114 | 0.727167 | 0.035879 | 0.280214 |
| PMS2P1 | 0.321547418 | 0.119618237 | 0.174610067 | 1.078122 | 1.200657 | 0.96999 | 0.533624 | 0.719095 |
| CWFI9L1 | 0.126052281 | 0.230846981 | 0.106858318 | 0.952501 | 1.483183 | 1.277709 | 0.330143 | 0.481491 |
| AP2S1 | 0.166481625 | 0.084924345 | 0.166281612 | 1.043069 | 1.453685 | 1.370528 | 0.4549 | 0.512857 |
| RAE1 | 0.28286054 | 0.039400847 | 0.057816643 | 0.53332 | 1.101733 | 0.339893 | −0.13741 | 0.547288 |
| TRIP12 | 0.437048772 | 0.397763158 | 0.532621914 | 0.613015 | 1.263028 | 0.700832 | 1.119876 | 0.533299 |
| PDZD11 | −0.239021064 | −0.350771285 | −0.248799786 | −0.00311 | 1.015932 | 0.220959 | 0.321041 | −0.50966 |
| SPG21 | −0.208881203 | −0.060474441 | −0.224861558 | 0.786815 | 1.519801 | 1.058161 | 0.744359 | 0.689506 |
| RRM1 | −0.138524821 | −0.07229604 | −0.365881984 | 0.225928 | 1.072332 | 0.344895 | 0.321387 | 0.816068 |
| SUB1 | −0.082327932 | −0.116445779 | −0.290623343 | 0.954789 | 1.43029 | 1.14927 | 0.624255 | 0.91965 |
| RAB11FIP1 | −0.086287348 | −0.23198829 | −0.107762887 | 0.629931 | 1.046704 | 0.799386 | 0.498719 | 0.138607 |
| USO1 | 0.191978511 | −0.155813619 | 0.012732572 | 1.400288 | 1.554749 | 1.768141 | 0.574276 | 0.688172 |
| NIPSNAP3A | −0.147489742 | −0.457481561 | −0.378928553 | 0.377759 | 1.013489 | 1.153516 | −0.09109 | 0.196009 |
| ANAPC13 | 0.419825911 | 0.025362257 | 0.106414706 | 1.084843 | 1.362232 | 1.186267 | −0.09475 | 0.555133 |
| AEN | −0.329911549 | −0.007373598 | −0.179018778 | 0.691636 | 1.660278 | 1.428005 | −0.08946 | 0.146419 |
| SF3B4 | 0.579410224 | 0.188193671 | 0.567372873 | 0.817178 | 1.296198 | 0.857227 | 0.225625 | 0.401738 |
| CAV1 | 0.808380987 | 0.342893188 | 0.804009388 | 0.530217 | 1.034845 | 0.746206 | 0.132075 | 0.166832 |
| PSPC1 | 0.063078268 | 0.234016597 | 0.764970712 | 0.557675 | 1.72325 | 1.406087 | −0.47231 | −0.95792 |
| TFRC | 0.712409468 | 0.594346373 | 0.745743458 | 0.771076 | 1.327545 | 1.239596 | −0.1216 | 0.087807 |
| WDR48 | 0.346354789 | 0.114268169 | 0.339313349 | 0.618686 | 1.153434 | 0.793528 | −0.84688 | −0.39236 |
| INO80C | 0.326443378 | 0.3815567 | 0.150512329 | 0.475378 | 1.11043 | 0.634701 | −0.04294 | −0.22729 |
| NOP58 | 1.278155484 | 1.099763895 | 1.168618849 | 2.037696 | 1.681631 | 1.211631 | −1.34739 | −1.8561 |
| NFAT5 | 0.622835758 | 0.675681518 | 0.675451383 | 1.615381 | 1.321585 | 1.065106 | −0.50348 | −0.91855 |
| LBH | 1.235360415 | 0.70916215 | 1.055238442 | 1.997106 | 1.977333 | 1.556413 | −0.29583 | −0.95394 |
| LMAN2 | 0.458426859 | 0.745441398 | −0.182106957 | 1.898264 | 1.905958 | 1.66741 | −0.81942 | −0.87735 |
| ACOT9 | −0.008340215 | 0.121073997 | −0.012702227 | 0.855439 | 1.264945 | 0.859329 | −0.21383 | −0.67938 |
| BRAP | 0.442194775 | 0.216922645 | 0.442668911 | 0.795537 | 1.212834 | 1.304609 | −0.15088 | −0.33959 |
| SLC7A5 | 0.660538816 | 0.69295036 | 1.130391468 | 0.377987 | 1.558707 | 1.248334 | −0.21503 | 0.353418 |
| CCT5 | 0.048549774 | 0.397884604 | 0.403965048 | 0.613356 | 1.661706 | 0.874976 | −0.54677 | 0.094678 |
| NAT10 | 0.179812273 | 0.070370031 | 0.428743783 | 0.323032 | 1.131739 | 0.769117 | −0.16187 | −0.22775 |
| YBX1 | 0.152518861 | 0.090029588 | 0.005221007 | 0.278663 | 1.812977 | 1.467586 | 0.066761 | 0.111679 |
| IMPDH2 | 0.531896428 | 0.130204872 | 0.164984586 | 0.757809 | 1.735492 | 1.879624 | −0.30275 | 0.13648 |
| PPM1B | 0.262638379 | 0.106989508 | −0.105862854 | 0.732445 | 1.543233 | 1.417317 | −0.82184 | −0.64094 |
| BANF1 | 0.235089878 | 0.583564828 | 0.149275382 | 0.818124 | 1.670338 | 0.809188 | 0.09579 | 0.551261 |
| PLEKHO2 | 0.031306885 | 0.245060463 | 0.054922269 | 1.242973 | 1.711002 | 1.649494 | 0.032681 | 0.121388 |
| HSPBP1 | 0.211751544 | 0.424298849 | 0.362168714 | 0.913504 | 1.14013 | 1.117104 | −0.16163 | 0.192993 |
| JTB | 0.142379785 | 0.392939178 | 0.617511262 | 0.732778 | 1.54531 | 0.992846 | −0.70944 | −0.7385 |
| SRA1 | 0.24406252 | 0.291462981 | 0.318596212 | 0.641769 | 1.108031 | 1.017807 | −0.59147 | −0.13662 |
| METTL9 | 0.186557939 | 0.451782276 | 0.332378961 | 0.629798 | 1.204562 | 0.827682 | −0.48782 | −0.34747 |
| SLC44A2 | −0.047167158 | 0.063241754 | 0.060539402 | 1.058063 | 0.942322 | 1.234281 | −0.84251 | −1.48286 |
| MYCBP | 0.304443034 | 0.343186647 | 0.234972751 | 0.542987 | 1.037817 | 0.782722 | −0.42572 | −0.61323 |
| KIAA0101 | 0.1015036 | 0.27973004 | 0.200646663 | −0.04911 | 1.640462 | 0.579242 | −0.56569 | 0.64925 |

| | Expression log2-ratio from comparison of high vs. low exhaustion cells in each tumor | | | | | | |
|---|---|---|---|---|---|---|---|
| | mel89 expression | mel74 expression | | | mel58 expression | | |
| | log-ratio | log-ratio | | | log-ratio | | |
| Gene | tumor/circulation | Mel75 | viral | tumor/circulation | Mel75 | viral | tumor/circulation |
| Names | (Baitch) | program | (Wherry) | (Baitch) | program | (Wherry) | (Baitch) |
| Consistent | | | | | | | |
| across | | | | | | | |
| tumors | | | | | | | |
| (FIG. 5E) | | | | | | | |
| CXCL13 | 3.608717 | 4.966735 | 4.168645 | 5.089142 | 3.598125 | 2.977387 | 3.134469 |
| TNFRSF1B | 1.920546 | 2.129356 | 0.417178 | 0.736088 | 2.449534 | 2.626307 | 2.112085 |
| RGS2 | 0.906373 | 3.233125 | 1.218876 | 2.372107 | 1.727185 | 1.158261 | 0.537784 |
| TIGIT | 1.17792 | 3.164345 | 2.173898 | 1.574072 | 1.585541 | 0.272803 | −0.29148 |
| CD27 | −0.49328 | 3.168417 | 2.116997 | 2.59768 | 3.424298 | 2.483798 | 2.846502 |
| TNFRSF9 | 0.046402 | 2.981633 | 1.536921 | 2.601022 | 2.416234 | 1.907534 | 1.637949 |
| SLA | 1.266932 | 2.909864 | 2.375121 | 2.758124 | −0.6464 | −1.07728 | −1.28305 |
| RNF19A | −0.23025 | 2.045523 | 1.568309 | 1.791634 | 1.720675 | 1.674719 | 1.022468 |
| INPP5F | 1.019338 | 2.281171 | 1.981053 | 2.404415 | 0.865835 | 0.85461 | 1.135519 |
| XCL2 | 1.185536 | 2.125218 | 0.36747 | 1.110474 | 0.787247 | 1.873101 | 2.622889 |
| HLA- | −0.20997 | 3.269884 | 1.646498 | 2.204549 | 2.346703 | 0.84874 | 1.884597 |
| DMA | | | | | | | |
| FAM3C | 0.525915 | 1.704999 | 1.598577 | 1.32675 | 1.546799 | 0.998554 | 0.360133 |
| UQCRC1 | 0.587658 | 1.436524 | 1.399558 | 1.323201 | 1.441773 | 1.108603 | 1.62452 |
| WARS | −0.54033 | 0.326702 | 0.820533 | 0.527008 | 2.354177 | 1.437824 | 1.827517 |
| EIF3L | 1.761054 | 1.84964 | 1.612293 | 1.405359 | 0.367139 | 0.555186 | 0.960215 |
| KCNK5 | 0.93295 | 1.814528 | 1.739655 | 1.954094 | 0.441106 | 1.127902 | 0.801895 |
| TMBIM6 | 0.338689 | 1.624363 | 0.256398 | 1.563375 | 0.088586 | 1.13048 | 0.017532 |
| CD200 | −0.42197 | 1.168482 | 0.882927 | 1.083176 | 2.198851 | 1.71636 | 0.548098 |
| ZC3H7A | 0.584778 | 0.777974 | 1.14011 | 1.209637 | 1.182607 | 1.725998 | 1.298049 |
| SH2D1A | 1.722098 | 2.451125 | 2.233419 | 1.861146 | −0.76876 | −0.43126 | 0.155181 |
| ATP1B3 | 0.012924 | 3.464061 | 2.777768 | 2.899307 | 0.891714 | −0.26178 | −0.62884 |
| MYO7A | 1.62883 | 1.386755 | 0.417893 | 1.273377 | 1.417572 | 1.374442 | 1.871448 |
| THADA | 0.801842 | 1.717367 | 1.298174 | 1.624631 | −0.28256 | −0.42438 | −0.50222 |
| PARK7 | 0.609988 | 0.269872 | 0.504409 | −0.06736 | 0.649883 | −0.18159 | −0.19731 |
| EGR2 | −0.63614 | 1.190273 | 1.054475 | 1.456022 | 1.008445 | 1.075675 | 1.008445 |
| FDFT1 | −0.62107 | 1.432249 | 1.607579 | 1.320907 | −0.26699 | −0.2086 | −0.10848 |
| CRTAM | 0.243626 | 2.57311 | 0.749008 | 1.64804 | −1.30646 | −1.60578 | −0.82105 |
| IFI16 | 1.296244 | −0.9009 | −1.56244 | −2.08241 | 1.125619 | 1.771556 | 1.68132 |
| variable | | | | | | | |
| across | | | | | | | |
| tumors | | | | | | | |
| (FIG. 5F) | | | | | | | |
| GMNN | 1.018215 | 0.533974 | 0.734214 | 0.425327 | 0.628446 | 0.347665 | 0.628446 |
| AFG3L1P | 0.912014 | 0.2751011 | −0.08768 | 0.831753 | 0.251979 | −0.11292 | 0.854899 |
| CSRP1 | 1.040596 | −0.28451 | 0.287429 | −0.40688 | −0.55576 | −0.48292 | 0.016909 |
| RBM5 | 1.802894 | 0.834414 | 0.133074 | 0.269213 | 0.469927 | 0.434452 | 1.419155 |
| AP1M1 | 2.362591 | 0.379448 | −0.42338 | 0.426055 | 0.777828 | 0.635288 | 0.714227 |
| NUCB2 | 1.455488 | 0.739028 | 0.486269 | 0.766003 | 0.940373 | 1.572182 | 1.294518 |
| NOP10 | 1.699537 | −0.00791 | −0.78168 | −0.50375 | 1.608849 | 1.817402 | 0.751008 |
| GFM1 | 1.265644 | 0.236237 | 0.045733 | 0.186358 | 1.167888 | 0.506536 | 0.707264 |
| DHRS7 | 1.575621 | 0.016951 | −0.70376 | −0.17341 | 0.585463 | 0.67419 | 0.804051 |
| SSU72 | 1.694657 | −0.52687 | −1.34791 | −0.81061 | 0.509338 | 0.991928 | 0.24116 |
| SBDS | 1.463692 | −0.58807 | −0.99137 | −0.79239 | −0.25048 | 0.683136 | −0.08241 |
| ATP6V1B2 | 1.113233 | −0.38281 | 0.122471 | 0.19029 | 0.258079 | 0.275284 | 0.258079 |
| VAPA | 0.973273 | 0.12649 | −0.2154 | −0.6692 | −0.06878 | 0.312473 | −0.40266 |
| CSNK2A1 | 0.542737 | 0.691363 | −0.21838 | 0.148314 | 0.253345 | 0.170453 | 0.093545 |
| LINC00339 | 0.58063 | −0.22603 | −0.34187 | 0.193466 | −0.15028 | −0.18367 | −0.16531 |
| MRPL4 | 1.009942 | 0.537733 | 0.316447 | 1.28217 | 0.942785 | −0.49219 | 0.044124 |
| PPP1R2 | 1.975633 | 1.081327 | 0.962823 | 0.517982 | 0.971954 | 0.593406 | 0.512116 |
| SMG1 | 1.088141 | 0.558574 | 0.408063 | −0.03249 | −0.20667 | −0.00176 | 0.008276 |
| OIP5- | 0.744279 | −0.22747 | −0.27997 | 0.530265 | −0.54316 | −0.58972 | −0.50957 |
| AS1 | | | | | | | |
| LPAR2 | 0.556391 | −0.32742 | 0 | −0.46948 | −2.25701 | −2.08535 | −1.29968 |
| LSMD1 | 0.848134 | 0.257991 | −0.46953 | −0.07916 | −1.15327 | −1.19504 | −0.83656 |
| STAG3L4 | 1.261516 | 0.180125 | 0.015755 | 0.22898 | −0.24467 | −0.35644 | −0.30604 |
| P4HB | 1.676497 | −0.04852 | 0.142006 | 0.617419 | −0.70932 | −0.65505 | −1.048 |
| SKP1 | 2.123037 | 0.926487 | −0.00439 | 1.390798 | 0.209082 | 0.745026 | −1.34026 |
| PTBP1 | 1.78723 | 0.515799 | 1.25224 | 0.785807 | −0.2323 | −0.46163 | −0.54447 |
| TSTA3 | 1.579474 | 1.408743 | 1.645046 | 0.970886 | −0.43247 | −0.52857 | −0.47571 |
| TBCB | 1.772263 | 1.186373 | 1.728763 | 2.296478 | 0.078168 | 0.156842 | −1.41376 |
| SMC5 | 1.035177 | 0.791623 | 0.977542 | 0.967109 | −0.10732 | 0.041372 | 0.426801 |
| KLHDC2 | 1.441406 | 0.766064 | 1.381456 | 0.869777 | −0.02752 | 0.171581 | 0.502961 |
| MPV17 | 1.760126 | 1.717559 | 1.175562 | 0.679983 | 0.111085 | 0 | 0.555167 |
| RBPJ | 1.747129 | 1.184692 | 0.811905 | 1.267026 | −0.08777 | −0.31264 | −0.30906 |
| POP5 | 1.087074 | 1.108069 | 0.54626 | 0.844056 | 0.816358 | 0.509102 | 0.060172 |
| PPAPDC1B | 0.900571 | 0.866762 | 0.666836 | 0.345315 | −1.01557 | −0.40361 | −0.48091 |
| IMP3 | 1.577398 | 1.518353 | 2.087736 | 1.769581 | −0.29227 | −0.34257 | −0.43657 |
| RNPS1 | 0.411595 | 0.525001 | 1.927725 | 0.993639 | −1.54561 | −1.7532 | −1.63823 |
| NFE2L2 | 0.290073 | 0.30563 | 1.064825 | 0.555934 | −1.54199 | −1.16451 | −1.04027 |
| SOD1 | 0.858406 | 1.634569 | 1.555276 | 1.231902 | −1.09581 | −1.67027 | −1.43689 |
| CD8B | 1.561027 | 0.885792 | 1.510959 | 0.237702 | −1.14475 | −0.9902 | −0.19162 |
| PTPN6 | 1.766931 | 2.500775 | 0.435532 | 1.174217 | −0.07013 | −0.31919 | 0.935582 |
| HSPA1B | 0.220985 | 2.575283 | 0.994634 | 2.239899 | −2.06659 | −1.05442 | −1.11229 |
| CD2BP2 | 0.457958 | 1.294905 | 0.8734 | 1.339214 | 0.384269 | 0.074944 | 0.387546 |
| ALDOA | 0.330086 | 1.049953 | 0.85658 | 0.470413 | 0.012205 | −1.05488 | 0.05869 |
| ZFP36L1 | 1.119544 | 0.952674 | 0.637601 | 1.185653 | 0.920901 | 0.515743 | 0.050209 |
| HSPB1 | 1.5024 | 2.656955 | 2.139988 | 2.541785 | 0.68804 | 0.650391 | −0.26544 |
| HSPA6 | 0.293233 | 1.297655 | 0.508111 | 1.294105 | 0 | 0 | 0 |
| ARHGEF1 | 0.46834 | 0.838824 | 0.49144 | 1.060869 | 0.637379 | 0.280885 | 0.141144 |
| LUC7L3 | 0.979735 | 1.385041 | 1.142914 | 1.022751 | 0.740808 | 0.845155 | 0.19573 |
| GPR174 | 0.338598 | 0.159402 | −0.40525 | 0.549965 | −1.01924 | −1.20287 | −1.49682 |
| ENTPD1 | 0.402213 | 1.509542 | 0.954705 | 0.898392 | −0.59507 | −0.27846 | −0.2505 |
| RASSF5 | 0.761281 | 1.729708 | 1.228719 | 2.011237 | 0.321849 | −0.19232 | 0.673079 |
| IPCEF1 | 0.289935 | 0.533911 | 0.962101 | 1.233604 | 0.400885 | 0.206357 | 0.041386 |
| ARNT | −0.23597 | 0.542681 | 0.526377 | 0.716853 | 0.341757 | −0.50869 | −0.53541 |
| NAB1 | 0.414185 | 1.25868 | 0.777166 | 1.194891 | 0.46894 | 0.068909 | 0.283564 |
| APLP2 | −0.28754 | 1.116532 | 0.610465 | 0.916647 | −0.05017 | −0.63529 | −0.04377 |
| PRKCH | −0.33548 | 2.121696 | 1.840446 | 1.759063 | 0.565513 | 0.529592 | 0.131581 |
| SEMA4A | −0.61867 | 2.060115 | 1.225383 | 1.220776 | 0.511601 | −0.23408 | 0.021306 |
| PPP1CC | −0.4417 | 2.138688 | 2.265622 | 2.905544 | −0.05101 | −0.52665 | −0.59551 |
| LAG3 | −0.1633 | 1.698212 | 1.610932 | 1.723443 | 0.795349 | −0.2956 | 0.07986 |
| HSPA1A | −1.08419 | 4.005502 | 2.733446 | 3.005388 | −0.65413 | −0.88562 | −0.53102 |
| SNAP47 | −0.06699 | 3.428857 | 2.631348 | 2.266286 | 0.323053 | 0.227684 | 0.454468 |
| CCL4L2 | −0.38391 | 3.299288 | 3.195172 | 2.832273 | −0.41126 | −0.47394 | −1.53097 |
| ARID4B | 0.230639 | 1.4386 | 1.133561 | 1.484528 | 0.413699 | 0.546384 | −0.03844 |
| LYST | −0.34794 | 1.889636 | 0.966794 | 1.073898 | −0.42279 | −1.12563 | −1.13817 |
| NMB | 0.402287 | 1.393265 | 1.114555 | 0.931411 | 0 | 0 | 0 |
| LIMS1 | 0.52875 | 0.932173 | 1.398524 | 0.996192 | 0.696663 | 0.245601 | 0.095847 |
| ITK | 0.117881 | 1.412365 | 2.007162 | 2.016882 | −0.4592 | −1.56794 | −1.12789 |
| RILPL2 | 0.128711 | 1.448139 | 1.378056 | 1.233603 | 0.201367 | −0.49364 | 0.330125 |
| RGS3 | 0.469319 | 0.766397 | 1.447569 | 0.77141 | 1.00919 | −0.42876 | 0.734015 |
| TRAT1 | −0.76925 | 1.739199 | 1.353801 | 1.292542 | 0.278364 | 0.138044 | 0.982559 |
| ELF1 | −0.78665 | 0.996661 | 0.408536 | 0.991955 | −0.62375 | 0.23482 | 0.491023 |
| OSBPL3 | 0.184624 | 1.099846 | 0.589255 | 0.844517 | 0.553114 | 0.490718 | 0.553907 |
| BIRC3 | −0.3585 | 1.064753 | 0.093569 | 0.572529 | 0.222131 | 0.089584 | −0.1936 |
| PTGER4 | −1.19825 | 0.358048 | 0.758733 | 0.556132 | −0.66042 | −0.93187 | −0.8841 |
| SERINC3 | −1.46498 | 1.90238 | 1.845857 | 1.06945 | −1.06771 | −0.24433 | −0.05405 |
| MED7 | −0.19409 | 0.420489 | 0.852556 | 1.294416 | 0.6235 | 0.524733 | 0.191409 |
| DDX3X | −0.26138 | 1.115431 | 2.036049 | 2.684364 | 0.991116 | 0.175224 | 0.067393 |
| THEM6 | −0.33051 | 0.80496 | 0.92413 | 1.295732 | 0.223017 | 0.237885 | −1.11E−16 |
| P4HA1 | −0.30807 | 0.691018 | 1.457828 | 1.790077 | 1.011478 | 0.831499 | 1.011478 |
| HIBCH | −0.76923 | 0.988116 | 1.028868 | 1.632567 | 0.748677 | 0.561102 | 0.384575 |
| VCAM1 | −0.11323 | 3.39384 | 2.14506 | 2.804809 | 1.560913 | 1.256303 | 1.174757 |
| FABP5 | 0.63961 | 3.741526 | 1.603507 | 1.782675 | 1.892396 | 0.21308 | 1.425971 |
| NOL7 | 9.19E−07 | 1.77296 | 2.60986 | 2.401256 | 0.444305 | −0.51709 | −0.02168 |
| SEC14L1 | 0.184813 | 0.862079 | 0.960584 | 1.016637 | 0.142151 | 0.151627 | 0.397834 |
| UBA2 | 0.611085 | 0.9791 | 1.469796 | 1.469796 | 0.224096 | 0.100813 | 0.171206 |
| CDCA4 | 0.884968 | 1.400085 | 0.900991 | 0.900991 | 0.041312 | 0.798032 | 0.662933 |
| ATP5I | 0.405697 | 1.157249 | 1.334829 | 2.488112 | 0.827137 | −0.05534 | −0.89279 |
| ALKBH3 | 0.211767 | 0.954907 | 1.379638 | 1.312997 | 0.51323 | 0.547446 | 0.079277 |
| DND1 | 0.233355 | 1.206843 | 1.377653 | 1.446083 | 0.397146 | 0.029691 | 0.276013 |
| RNF185 | −0.03906 | 0.723283 | 1.168166 | 1.25853 | −0.15047 | −0.10052 | 0.329688 |
| AFAP1L2 | 0.541622 | 1.720377 | 1.965136 | 2.135432 | 0.479959 | 0.312523 | 0.186969 |
| GLOD4 | 0.591136 | 2.21587 | 1.669596 | 2.753296 | 0.255249 | 0.151885 | 0.578215 |
| PIP5K1A | 0.206298 | 1.203715 | 1.362138 | 1.427971 | −0.20775 | 0.052328 | 0.652579 |
| ATF4 | 0.934837 | 2.478102 | 2.401872 | 2.923167 | 0.404639 | −0.30419 | 0.760243 |
| PIGO | 0.344022 | 0.989226 | 1.294802 | 1.360186 | 0.29352 | 0 | 0.29352 |
| OPA1 | 0.130641 | 0.968233 | 0.960402 | 1.004096 | 0.092508 | 0.090458 | 0.215837 |
| CCT3 | 0.057483 | 2.558462 | 2.987783 | 3.096527 | −0.39291 | −0.53424 | 0.092772 |
| EXOSC6 | −0.09089 | 1.049513 | 1.713485 | 1.463248 | −0.77445 | −0.39914 | −0.28299 |
| KIAA1429 | −0.03324 | 0.601999 | 1.146362 | 1.460962 | −0.34572 | 0.447705 | −0.265 |
| NDFIP2 | 0.702691 | 1.554856 | 1.422824 | 1.841077 | 0.885623 | 0.944664 | 0.096071 |
| TMEM222 | −0.01843 | 1.043619 | 0.595152 | 1.883823 | 0.033681 | −0.64885 | −0.58678 |
| MYO1G | 0.264914 | 2.159652 | 2.450084 | 3.119571 | −1.34234 | −2.22574 | −1.35433 |
| LBR | 0.837118 | 1.435512 | 1.660001 | 2.290788 | −0.65948 | −0.65997 | −0.75644 |
| EXT2 | 0.780516 | 0.89716 | 1.020431 | 1.020431 | 0.251034 | −0.13385 | −0.29829 |
| SARDH | 0.945961 | 1.326633 | 1.080222 | 1.808359 | 0.919535 | 0.303024 | −0.06293 |
| POLR2I | 0.289083 | 0.901354 | 1.294324 | 1.78872 | −0.517 | −0.30553 | −0.20034 |
| HNRNPD | 1.221674 | 1.314644 | 2.247061 | 2.528336 | 0.693014 | 0.258226 | 0.224703 |
| NAAA | −0.17928 | 1.217255 | 1.388832 | 1.621152 | −0.25806 | −0.2745 | −0.08237 |
| ARID5A | 0.339123 | 2.299884 | 2.585353 | 2.65627 | 0.882872 | 0.511331 | 1.126729 |
| PDRG1 | 0.054912 | 1.113032 | 1.530419 | 1.530419 | 0.390793 | 0.416846 | 0.390793 |
| BCAP31 | 1.400717 | 2.292282 | 2.018457 | 2.510231 | 1.661388 | 1.693049 | 0.800257 |
| UQCRFS1 | 0.901798 | 1.796312 | 1.631373 | 2.23279 | 1.442821 | 1.338512 | 1.442821 |
| SNRNP40 | 0.458705 | 1.228688 | 1.015203 | 2.151944 | 1.951393 | 1.514 | 1.548637 |
| ASB8 | 0.278134 | 0.694184 | 1.168198 | 1.253288 | 1.155424 | 1.285273 | 1.45607 |
| MRPL52 | −0.26895 | 0.776358 | 1.358309 | 1.632777 | 1.089123 | 0.899545 | 1.185497 |
| TUG1 | 0.543653 | 0.971814 | 1.254514 | 1.791286 | 0.762382 | 0.950826 | 1.017822 |
| CCND2 | 0.477816 | 1.567577 | 1.781844 | 2.317427 | 1.200583 | 1.790085 | 0.894407 |
| NAA20 | 0.114279 | 1.877634 | 2.336312 | 2.679865 | 1.734987 | 1.088804 | 0.99408 |
| HLA- | 0.48986 | 3.627783 | 2.731242 | 3.451219 | 2.964099 | 1.893692 | 2.451944 |
| DPA1 | | | | | | | |
| TOX | 0.39047 | 2.750289 | 2.547279 | 3.326697 | 1.552835 | 1.44462 | 2.682133 |
| TMEM205 | −0.17865 | 0.819502 | 1.126816 | 1.803619 | 0.810292 | 1.283092 | 0.810292 |
| TPI1 | 0.428852 | 1.757871 | 0.502927 | 1.333332 | 2.39651 | 2.035743 | 1.244824 |
| HADHA | 0.583335 | 1.025366 | 1.032751 | 1.19827 | 1.867333 | 0.79595 | 1.975448 |
| STAT3 | 0.911609 | 0.953001 | 1.360722 | 0.369746 | 1.246119 | 1.320042 | 1.918188 |
| GMDS | −0.00189 | 0.731454 | 0.363281 | 0.684468 | 2.20812 | 2.070111 | 2.138562 |
| SIRPG | 0.999991 | 0.669789 | 0.457155 | 0.971561 | 2.482767 | 2.725171 | 4.320541 |
| ITM2A | 1.238039 | 1.195596 | 0.083568 | 1.192628 | 2.155813 | 1.908674 | 3.926131 |
| TBC1D4 | 0.528543 | 0.446225 | −0.12284 | −0.11 | 1.408485 | 1.572206 | 1.012914 |
| HNRNPM | −0.76372 | −0.86522 | −1.09826 | −0.66369 | 0.649506 | 0.980231 | 0.252941 |
| ASB2 | 0.553956 | 0.440061 | 0.442262 | 0.507727 | 1.271536 | 0.780069 | 1.18805 |
| IGFLR1 | 0.279285 | 1.316036 | −0.66578 | 0.102396 | 2.87465 | 3.060404 | 3.296175 |
| CD2 | −0.01697 | 0.491908 | −0.03799 | 0.032518 | 1.717787 | 2.777401 | 1.167452 |
| COTL1 | −0.38639 | −0.39007 | −0.74662 | −0.7776 | 4.006922 | 4.063247 | 2.891143 |
| PBRM1 | 0.334139 | 0.113374 | 0.128701 | −0.16289 | 0.912786 | 1.069835 | 0.568188 |
| DUT | 0.729157 | 0.361399 | −0.82348 | −0.31462 | 1.493089 | 1.024494 | 2.020066 |
| LMF2 | 0.423231 | −0.18509 | −0.58873 | −0.29092 | 0.728777 | 0.507645 | 1.5803 |
| TAF15 | 0.635818 | 0.131015 | −0.33584 | −0.08184 | 0.982932 | 0.813021 | 0.863234 |
| H2AFY | 1.45784 | −0.42473 | −0.58958 | −0.60627 | 0.693423 | 0.688452 | 0.803592 |
| CEP57 | 0.672848 | −0.44914 | −0.75179 | −0.4288 | 0.854391 | 0.934846 | 0.960197 |
| AMDHD2 | 0.442078 | −0.52913 | −0.83302 | −0.53238 | 0.712842 | 1.258587 | 0.322542 |
| SERINC1 | 1.502882 | 0.330365 | −0.55982 | −0.09584 | 0.924355 | 0.826487 | 1.263562 |
| CKS2 | 0.694436 | 0.299497 | 0.165571 | 0.079421 | 0.835914 | 0.112142 | 1.17671 |
| PTPN11 | 1.057707 | 0.745506 | 0.103679 | −0.10591 | 1.336193 | 0.497775 | 0.993959 |
| DDX3Y | 0.348148 | −0.61359 | −0.59356 | −0.70761 | 0.416104 | 0.772211 | −0.02582 |
| IRF9 | 1.031441 | −0.6564 | −1.22673 | −1.69271 | −0.32663 | −0.32355 | 0.537973 |
| FYN | 0.833936 | −0.17033 | −1.12408 | −1.82722 | −1.32784 | −1.28945 | −0.45365 |
| HSPD1 | 1.202617 | 0.37959 | −0.9499 | −0.24297 | 0.555444 | −0.34555 | 0.154972 |
| FPGS | 0.407362 | 0.423797 | 0.37676 | 0.470761 | 0.194845 | 0.030908 | 0.792577 |
| CCT2 | 0.654477 | 0.834471 | 0.08622 | −0.35726 | −0.49579 | −0.10421 | −0.19894 |
| GNAS | 0.902383 | 1.48214 | −0.36497 | −0.11729 | 0.139607 | −1.09785 | −1.15442 |
| FAIM3 | 0.198604 | −0.20562 | −0.78752 | −0.4545 | 0.084154 | −0.34378 | −1.5106 |
| ETV1 | 0.670267 | 0.847637 | 0.254564 | 0.254339 | 0.454184 | −0.02805 | −0.34273 |
| BCL6 | 0.561538 | 0.239024 | 0.164883 | 0.164883 | −0.02492 | −0.0663 | −0.05046 |
| SLC38A1 | 0.683811 | −0.07354 | −0.35771 | 0.030638 | −0.01663 | −0.132 | −1.01087 |
| PDE7B | 0.706219 | 0.068219 | 0.104437 | −0.23208 | −0.54377 | −0.23153 | −0.00788 |
| STAT1 | −0.18625 | −0.60879 | −0.8852 | −1.43629 | −0.21528 | −2.42559 | −0.93656 |
| EIF3H | 0.583077 | −0.56724 | −0.27437 | 0.040546 | 1.018019 | −0.93179 | −0.87078 |
| EID1 | 1.342076 | 0.903437 | −0.38021 | 0.74738 | 0.126626 | −0.9091 | −0.67118 |
| ID3 | 1.150499 | 1.094383 | −0.52374 | 0.110707 | −0.61883 | −0.12334 | −0.90445 |
| PSAP | 1.015173 | −0.08822 | 0.357411 | −0.30742 | 0.033676 | −0.63389 | −0.37108 |
| DPP7 | 0.881582 | 0.264034 | 0.478359 | 0.712926 | 0.325353 | −0.23218 | 0.729856 |
| PJA2 | 1.578818 | 0.619118 | −0.01441 | 0.252184 | −0.00144 | 0.345169 | −0.54049 |
| TARDBP | 0.676963 | −0.10032 | −0.14551 | 0.205089 | 1.10712 | −0.02032 | −0.46061 |
| SRSF1 | 0.690731 | −0.04179 | 0.742665 | 0.225246 | 0.760135 | 0.619198 | 0.040697 |
| GABPB1 | −0.25196 | −0.05844 | 0.140932 | 0.266118 | 0.613771 | 0.349438 | −0.49602 |
| RGS4 | 0.236492 | 0.405497 | 0.222587 | 0.541634 | 1.000554 | 0.636924 | 0.325281 |
| SPTAN1 | −0.57718 | 0.35581 | −0.02982 | 0.062045 | 0.025741 | −0.31616 | −0.25387 |
| NFATC1 | 0.571233 | 0.868881 | 0.869066 | 0.765583 | 0.57723 | 0.403698 | 0.988219 |
| HAVCR2 | −0.33104 | 2.137501 | 1.022968 | 1.834091 | 1.159592 | 0.250215 | 1.202646 |
| PDCD1 | −0.66284 | 3.144575 | 1.775263 | 1.89359 | 2.157837 | 1.426781 | 1.292126 |
| SRSF4 | −1.56888 | 0.268312 | −0.39881 | −0.12716 | 1.217071 | 1.61926 | 1.543732 |
| GFOD1 | −0.01037 | 0.690616 | 0.666958 | 0.72558 | 1.279323 | 1.191165 | 1.70711 |
| MRPS21 | −0.20862 | 0.63153 | 0.640025 | 1.099578 | 0.782744 | 1.047579 | 2.076158 |
| AP3S1 | −0.33404 | 0.896161 | −0.00414 | 0.352526 | 0.876832 | 0.490312 | 0.833394 |
| GPBP1 | −2.01114 | 0.114516 | 0.059784 | 0.099757 | −0.11017 | −0.15826 | 0.560546 |
| BTLA | −0.61107 | 0.808268 | 0.737358 | 0.990409 | 0.849099 | 0.94081 | 0.419206 |
| PAM | −1.05575 | 1.082388 | 0.960187 | 0.97289 | 0.561424 | 0.797694 | 0.019995 |
| CBLB | 0.14064 | 1.86455 | 0.546329 | 1.031677 | 1.762483 | 1.250389 | 1.264264 |
| ATHL1 | 0.012087 | 1.614496 | 0.813458 | 0.887073 | 1.56931 | 0.821076 | 1.508315 |
| MGEA5 | 0.178722 | 0.246747 | 0.076476 | −0.53816 | 1.021961 | 1.189852 | 0.964678 |
| IRF4 | −0.10278 | −0.08757 | 0.015939 | −0.0309 | 1.026245 | 0.771419 | 1.120829 |
| UBE2F | 0.445397 | 0.72288 | −0.38748 | −0.16617 | 0.693354 | 0.555277 | 1.257862 |
| SFXN1 | −0.16715 | 0.400925 | 0.167878 | 0.046547 | 0.601289 | 0.852965 | 1.083502 |
| DGKH | 0.237762 | 0.301583 | 0.434966 | 0.217005 | 0.847039 | 0.606389 | 0.699954 |
| FCRL3 | −0.25236 | 0.459938 | −0.69099 | −0.6725 | 1.030386 | 0.806411 | 1.727726 |
| PYHIN1 | −0.4332 | 0.32291 | −0.91689 | −0.51119 | −0.97342 | 0.387719 | −0.19257 |
| EIF1B | −0.80839 | 0.498479 | −0.44684 | −0.49147 | 0.424981 | 0.387659 | 0.082507 |
| RAPGEF6 | −0.25743 | 0.177709 | 0.42636 | 0.513435 | 0.407581 | 0.856307 | 0.319354 |
| SNX9 | 0.693705 | 0.534062 | 0.404421 | 0.669603 | 0.881685 | 0.772082 | 0.677458 |
| IL6ST | 0.827538 | −0.02703 | −0.15832 | −0.40796 | 0.608931 | 0.425259 | 0.118486 |
| PTPN7 | 0.202085 | 1.03909 | −1.31399 | −0.08723 | 0.964107 | 0.27589 | 0.140967 |
| CREM | −0.1493 | −0.15654 | −1.59053 | −1.41624 | 0.866148 | 0.050263 | −0.09383 |
| HNRPLL | 0.005445 | 0.11502 | −0.48435 | −0.70676 | 0.51347 | 0.737323 | 0.623868 |
| FUT8 | −0.87225 | −0.15132 | −1.4016 | −0.92911 | −0.2844 | −0.45036 | 0.036153 |
| LITAF | −1.16087 | 0.121907 | −0.75742 | −0.11928 | 0.122899 | −0.73399 | −2.42571 |
| TSC22D1 | 0.030462 | 0.465096 | 0.423067 | 0.211233 | 0 | 0 | 0 |
| TRAF5 | 0.342506 | 0.9409 | 0.947122 | 0.939609 | 0.386643 | 0.832158 | 0.713993 |
| ATP6V0B | −0.08735 | −0.32232 | 0.241701 | 0.062118 | 0.11099 | −1.0389 | −0.03137 |
| SRSF6 | −0.79042 | 0.242284 | −0.17696 | 0.055511 | 0.156616 | −1.10955 | −0.4541 |
| ELMO1 | −0.29337 | 0.327883 | −0.02779 | 0.222429 | 0.040869 | −0.25369 | −0.36432 |
| IRF8 | 0.296565 | 0.310499 | 0.320116 | 0.457066 | 0.408175 | 0.340339 | −0.00824 |
| TAGAP | −1.59095 | −0.92531 | −1.73693 | −0.79445 | 0.0226 | −1.42961 | −1.1276 |
| CADM1 | −0.38593 | 0.156512 | 0.186461 | −0.10824 | 0.003477 | 0.126302 | 0.118408 |
| SPRY2 | −0.05141 | 0.355988 | −0.25618 | 0.175899 | −0.28658 | −0.61825 | −0.33716 |
| CTLA4 | 0.293249 | 1.288938 | 0.218396 | 0.707927 | −0.05016 | −0.4268 | 0.075253 |
| ANKRD10 | −0.19038 | 0.44642 | −0.60352 | −0.41603 | −0.87124 | −0.76427 | −0.81685 |
| KLRK1 | −0.10713 | 0.322315 | −0.15817 | −0.40615 | −0.70791 | −0.78216 | −1.85237 |
| TP53INP1 | 0.299783 | 0.853731 | 0.66009 | 0.778619 | 0.114029 | −0.8017 | −0.81122 |
| NR4A2 | −0.2557 | 1.124934 | −0.1027 | 0.466739 | −1.16197 | −1.62068 | −1.88389 |
| ZNF292 | 0.070993 | 0.361044 | 0.616271 | 0.319476 | −0.67403 | −1.16473 | −0.85531 |
| MIF4GD | 0.67596 | −0.1032 | 0.23025 | −0.07087 | −0.63027 | −1.53555 | −0.91825 |
| ING3 | −0.15693 | −0.11028 | −0.79956 | −0.69731 | −0.34622 | −1.37596 | −1.25438 |
| SQSTM1 | 0.169825 | 0.782132 | −0.88949 | −0.09323 | −0.98047 | −0.68004 | 0.227808 |
| CLK4 | −0.31286 | −0.36076 | −0.99938 | −0.64202 | −0.4513 | −0.35291 | −0.31603 |
| NCBP2 | 0.017456 | 0.058449 | −0.56375 | 0.057907 | 0.370549 | −0.10644 | 0.360072 |
| SET | 0.135136 | −0.5062 | −0.59682 | 0.044981 | 0.553899 | 1.28517 | 0.423553 |
| PSME3 | 0.033817 | 0.50342 | −0.29391 | −0.10906 | 0.297055 | 0.734865 | 0.297055 |
| IQCB1 | −0.337 | −0.24326 | −0.96163 | −0.50434 | −0.44439 | 0.163647 | −0.10429 |
| RGCC | 0.187399 | −0.36292 | −0.21782 | −0.22329 | −0.26213 | 0.707684 | 0.116887 |
| C20orf111 | −0.48954 | −0.1712 | −0.57642 | −0.12684 | −1.11E−16 | −1.11E−16 | −1.11E−16 |
| MPP1 | 0.117783 | 0.268042 | −0.06759 | 0.019518 | 0.695404 | 0.741764 | 0.695404 |
| CALR | −1.2216 | −0.6414 | −0.31174 | −0.87079 | 1.071562 | −0.22387 | 0.420225 |
| TMEM160 | −0.14405 | 0.374662 | −0.178 | −0.43914 | 1.174904 | −0.26706 | 0.125822 |
| SRGN | 0.61632 | 2.086102 | 0.331426 | 0.633613 | 1.973977 | 1.286672 | 0.530717 |
| EWSR1 | −0.66469 | 0.244222 | −0.95547 | −1.2807 | 1.386017 | −0.15908 | 1.162643 |
| EZR | 0.002138 | −0.25954 | −0.80189 | −1.7707 | 0.415785 | 0.526208 | 1.065046 |
| FTSJ3 | 0.09123 | −0.48372 | −0.75763 | −0.55904 | 0.194814 | 0.207802 | 0.194814 |
| LRMP | 0.335465 | 0.181242 | −0.8705 | −0.37811 | 0.913822 | 0.606969 | 0.891155 |
| GBP2 | 1.350823 | 0.753661 | 0.605278 | 0.14575 | 2.507574 | 1.30646 | 2.636831 |
| MPG | −0.33026 | −0.3406 | −0.08421 | −1.1428 | 0.517121 | 1.206441 | 0.45573 |
| RELA | 0.729125 | −0.37902 | −0.39609 | −0.49493 | 0.145874 | 0.96174 | 0.200113 |
| KLHDC4 | 0.535737 | −0.63391 | −0.88522 | −0.60859 | 0 | 0 | 0 |
| PMS2P1 | 0.440142 | −0.25173 | −0.20455 | −0.40305 | −0.10217 | −0.29901 | 0.022447 |
| CWFI9L1 | 0.421855 | −0.14695 | 0.327528 | −0.27353 | −0.15851 | −0.02253 | −0.40174 |
| AP2S1 | 0.018165 | −0.26256 | −0.9045 | −0.20552 | −1.40231 | −1.63028 | −1.43902 |
| RAE1 | 0.303881 | 0.012973 | −0.28942 | 0.123725 | −0.67669 | −0.88066 | −0.24716 |
| TRIP12 | 0.623553 | 0.371347 | 0.416789 | 0.388284 | 0.046328 | −0.39998 | 0.386772 |
| PDZD11 | 0.088924 | 0.164688 | −0.3767 | −0.25898 | −0.618 | −0.91664 | −0.50696 |
| SPG21 | 0.635645 | 0.752776 | 0.033819 | 0.215925 | 0.121577 | 0.283619 | −0.18976 |
| RRM1 | 0.047308 | 0.110234 | 0.044488 | 0.307286 | −0.07085 | −0.12007 | −0.01679 |
| SUB1 | 0.202585 | 0.765195 | −0.34261 | 0.827681 | 0.773933 | 0.19674 | 0.548024 |
| RAB11FIP1 | 0.278209 | 0.345668 | 0.20844 | 0.696345 | 0.001646 | −0.11654 | 0.272953 |
| USO1 | 0.831964 | 0.315983 | 0.52313 | 0.696388 | −0.1749 | −0.36653 | 0.471195 |
| NIPSNAP3A | 0.006116 | −0.35987 | −0.21365 | 0.187133 | −5.55E−17 | −5.55E−17 | −5.55E−17 |
| ANAPC13 | 0.502264 | 0.152503 | 0.528539 | 0.528539 | 0.381303 | 0.406723 | 0 |
| AEN | 0.136278 | −0.32053 | 0.777784 | −0.45779 | −0.1511 | −0.50862 | −0.28785 |
| SF3B4 | 0.421259 | 0.339652 | 0.801295 | 0.349043 | 0.21863 | 0.035141 | −0.12023 |
| CAV1 | 0.150943 | 0.26682 | 0.429648 | 0.429648 | 0 | 5.55E−17 | 5.55E−17 |
| PSPC1 | −0.95732 | 0.904459 | −0.0455 | 0.103508 | −1.60427 | −0.89935 | −1.42763 |
| TFRC | −0.34992 | 0.86135 | 0.102911 | 0.379846 | 0.191531 | 0.149762 | −0.22741 |
| WDR48 | −1.0368 | 0.28922 | −0.29768 | −0.10897 | −0.29522 | 5.55E−17 | −0.32475 |
| INO80C | −0.00682 | 0 | 0.217444 | 0.151339 | 0 | 0 | 0 |
| NOP58 | −2.02212 | 0.157164 | 0.452792 | 0.004688 | 0.018164 | −0.05158 | −0.3837 |
| NFAT5 | −0.20388 | 0.915999 | 0.288399 | 0.725247 | 0.404725 | 0.589463 | 0.385799 |
| LBH | −0.82223 | 0.945329 | 1.377524 | 1.19642 | 1.208219 | 0.381495 | −0.26731 |
| LMAN2 | −0.7033 | 0.809343 | 1.42215 | 1.367199 | 1.344057 | 1.049097 | −0.33008 |
| ACOT9 | −0.89228 | 0.216613 | 0.932709 | 0.518503 | −0.0465 | 0.245332 | −0.0886 |
| BRAP | −0.03363 | 0.65607 | 0.864995 | 0.631052 | 0.278686 | 0.029397 | 0.401201 |
| SLC7A5 | −0.20594 | 1.478834 | 1.013417 | 0.669421 | 0.449961 | 0.100231 | 1.014548 |
| CCT5 | −0.29939 | 0.718911 | 0.719806 | 0.230193 | 0.23639 | −0.13455 | 0.183522 |
| NAT10 | 0.031164 | 0.684485 | 0.210096 | 0.320506 | 0.013249 | 0.014026 | 0.013149 |
| YBX1 | −0.20106 | 0.128291 | 0.418769 | 0.785448 | 0.279589 | 0.077383 | 0.075305 |
| IMPDH2 | −0.01746 | 0.507119 | 0.410634 | 0.7115 | 0.074426 | −0.00574 | 0.316778 |
| PPM1B | −0.53333 | −0.8919381 | 0.7132 | 0.649096 | −0.61055 | −1.07683 | −1.02442 |
| BANF1 | 0.033987 | 0.884743 | 0.61913 | 1.170524 | −0.27021 | −0.73039 | −0.01189 |
| PLEKHO2 | −0.08886 | 1.010773 | 1.160452 | 1.30773 | −0.34891 | 0.155771 | 0.130711 |
| HSPBP1 | −5.55E−17 | 0.684745 | 0.581124 | 1.293263 | 0.344035 | 0.128045 | 0.223993 |
| JTB | 0.026636 | 0.944317 | 2.419671 | 1.557654 | 0.943213 | −0.37932 | 0.63405 |
| SRA1 | −0.1116 | 1.191187 | 1.413605 | 0.948158 | 0.544012 | 0.863391 | 0.341727 |
| METTL9 | −0.44601 | 1.321009 | 0.920825 | 1.020634 | 0.666998 | 0.354146 | 0.666998 |
| SLC44A2 | −0.57768 | 0.651139 | 1.279204 | 1.406301 | 1.094011 | 0.886149 | 1.367995 |
| MYCBP | −0.53543 | 0.409915 | 0.202636 | 0.310912 | 0.651789 | 0.695241 | 0.651789 |
| KIAA0101 | 0.100417 | 0.317965 | 0.375401 | 0.627312 | 0.618515 | 0.58803 | 1.153147 |

| | P-values from comparison of high vs. low exhaustion cells in each tumor | | | | | | | | |
|---|---|---|---|---|---|---|---|---|---|
| | mel75 p-value | | | mel79 p-value | | | mel89 p-value | | |
| | | | tumor/ | | | tumor/ | | | tumor/ |
| Gene | Mel75 | viral | circulation | Mel75 | viral | circulation | Mel75 | viral | circulation |
| Names | program | (Wherry) | (Baitch) | program | (Wherry) | (Baitch) | program | (Wherry) | (Baitch) |
| Consistent | | | | | | | | | |
| across | | | | | | | | | |
| tumors | | | | | | | | | |
| (FIG. 5E) | | | | | | | | | |
| CXCL13 | 0 | 0 | 0 | 0.0237 | 0.0561 | 0.0086 | 0 | 0.0003 | 0.0006 |
| TNFRSF1B | 0 | 0 | 0 | 0 | 0 | 0 | 0.001 | 0.0168 | 0.0089 |
| RGS2 | 0 | 0 | 0 | 0.0056 | 0.11 | 0.143 | 0.0994 | 0.0152 | 0.177 |
| TIGIT | 0 | 0 | 0 | 0.0007 | 0.0016 | 0.0005 | 0.1611 | 0.0278 | 0.0665 |
| CD27 | 0 | 0 | 0 | 0.0438 | 0.2998 | 0.3121 | NaN | NaN | NaN |
| TNFRSF9 | 0 | 0 | 0 | 0 | 0.0018 | 0.0131 | NaN | NaN | NaN |
| SLA | 0 | 0 | 0 | 0 | 0.0012 | 0.0005 | 0.0015 | 0.0232 | 0.0587 |
| RNF19A | 0 | 0 | 0 | 0.0015 | 0.0631 | 0.0184 | NaN | NaN | NaN |
| INPP5F | 0 | 0 | 0 | 0.006 | 0.0029 | 0.0036 | 0.036 | 0.1813 | 0.0318 |
| XCL2 | 0.0004 | 0.014 | 0.0058 | 0.0379 | 0.0027 | 0.0003 | 0.1265 | 0.0147 | 0.0691 |
| HLA- | 0 | 0.0146 | 0 | 0.0424 | 0.0156 | 0.201 | NaN | NaN | NaN |
| DMA | | | | | | | | | |
| FAM3C | 0 | 0 | 0 | 0.0008 | 0.0022 | 0.0341 | NaN | NaN | NaN |
| UQCRC1 | NaN | NaN | NaN | 0.0243 | 0 | 0.0025 | 0.2879 | 0.0135 | 0.2424 |
| WARS | 0 | 0.0018 | 0.0004 | 0.0014 | 0.0008 | 0.0008 | NaN | NaN | NaN |
| EIF3L | 0.0287 | 0.0071 | 0.0047 | 0.4328 | 0.0658 | 0.3026 | 0.4936 | 0.0008 | 0.0138 |
| KCNK5 | 0 | 0.0011 | 0.0029 | NaN | NaN | NaN | 0.0052 | 0.0303 | 0.0288 |
| TMBIM6 | 0 | 0.0809 | 0.0034 | 0.0009 | 0.0136 | 0.0006 | 0.0904 | 0.1625 | 0.339 |
| CD200 | 0 | 0 | 0.0001 | 0.0007 | 0.0513 | 0.0259 | NaN | NaN | NaN |
| ZC3H7A | 0 | 0 | 0 | NaN | NaN | NaN | NaN | NaN | NaN |
| SH2D1A | 0.001 | 0.0155 | 0.0291 | NaN | NaN | NaN | 0.0306 | 0.0004 | 0.0111 |
| ATP1B3 | 0.0021 | 0.0471 | 0.0042 | 0.574 | 0.0316 | 0.1129 | NaN | NaN | NaN |
| MYO7A | NaN | NaN | NaN | NaN | NaN | NaN | 0.003 | 0.0009 | 0.0003 |
| THADA | 0 | 0.0002 | 0 | NaN | NaN | NaN | 0.0587 | 0.0052 | 0.0309 |
| PARK7 | 0.0003 | 0 | 0 | 0.4814 | 0.0163 | 0.1843 | 0.3094 | 0.0464 | 0.2595 |
| EGR2 | 0 | 0.0016 | 0.0015 | 0.0689 | 0.0029 | 0.0499 | NaN | NaN | NaN |
| FDFT1 | 0.0001 | 0.0007 | 0.0004 | 0.2754 | 0.0426 | 0.026 | NaN | NaN | NaN |
| CRTAM | 0.0008 | 0.0541 | 0.0001 | 0.0972 | 0.0222 | 0.0014 | NaN | NaN | NaN |
| IFI16 | 0.0001 | 0.0013 | 0.0027 | NaN | NaN | NaN | 0.0163 | 0.0297 | 0.0362 |
| variable | | | | | | | | | |
| across | | | | | | | | | |
| tumors | | | | | | | | | |
| (FIG. 5F) | | | | | | | | | |
| GMNN | NaN | NaN | NaN | NaN | NaN | NaN | 0.6228 | 0.0008 | 0.0132 |
| AFG3L1P | NaN | NaN | NaN | NaN | NaN | NaN | 0.058 | 0.0001 | 0.0064 |
| CSRP1 | NaN | NaN | NaN | NaN | NaN | NaN | 0.0737 | 0.0008 | 0.0309 |
| RBM5 | NaN | NaN | NaN | NaN | NaN | NaN | 0.0014 | 0 | 0.0014 |
| AP1M1 | NaN | NaN | NaN | NaN | NaN | NaN | 0.0033 | 0 | 0 |
| NUCB2 | NaN | NaN | NaN | NaN | NaN | NaN | 0.0072 | 0.0005 | 0.0107 |
| NOP10 | NaN | NaN | NaN | NaN | NaN | NaN | 0.1509 | 0 | 0.0092 |
| GFM1 | NaN | NaN | NaN | NaN | NaN | NaN | 0.1149 | 0.0004 | 0.0024 |
| DHRS7 | NaN | NaN | NaN | NaN | NaN | NaN | 0.0408 | 0.0007 | 0.0144 |
| SSU72 | NaN | NaN | NaN | NaN | NaN | NaN | 0.002 | 0.0001 | 0.0051 |
| SBDS | NaN | NaN | NaN | NaN | NaN | NaN | 0.0372 | 0.0003 | 0.0008 |
| ATP6V1B2 | NaN | NaN | NaN | NaN | NaN | NaN | 0.1751 | 0 | 0.0024 |
| VAPA | NaN | NaN | NaN | NaN | NaN | NaN | 0.005 | 0.0004 | 0.0284 |
| CSNK2A1 | NaN | NaN | NaN | NaN | NaN | NaN | 0.1584 | 0.0006 | 0.1434 |
| LINC00339 | NaN | NaN | NaN | NaN | NaN | NaN | 0.1008 | 0.0005 | 0.059 |
| MRPL4 | NaN | NaN | NaN | NaN | NaN | NaN | 0.0904 | 0.001 | 0.0312 |
| PPP1R2 | NaN | NaN | NaN | 0.1222 | 0.0004 | 0.0003 | 0.0368 | 0.0127 | 0.0018 |
| SMG1 | NaN | NaN | NaN | NaN | NaN | NaN | 0.0304 | 0.0006 | 0.0041 |
| OIP5- | NaN | NaN | NaN | 0.0301 | 0.0058 | 0.0028 | 0.0057 | 0.0003 | 0.0194 |
| AS1 | | | | | | | | | |
| LPAR2 | NaN | NaN | NaN | NaN | NaN | NaN | 0.3197 | 0.0004 | 0.1294 |
| LSMD1 | NaN | NaN | NaN | NaN | NaN | NaN | 0.0015 | 0.0003 | 0.0351 |
| STAG3L4 | NaN | NaN | NaN | NaN | NaN | NaN | 0.0065 | 0 | 0.0005 |
| P4HB | NaN | NaN | NaN | NaN | NaN | NaN | 0.1486 | 0.0004 | 0.0146 |
| SKP1 | NaN | NaN | NaN | NaN | NaN | NaN | 0.0076 | 0.001 | 0.0026 |
| PTBP1 | NaN | NaN | NaN | NaN | NaN | NaN | 0.0147 | 0.0001 | 0.0014 |
| TSTA3 | NaN | NaN | NaN | NaN | NaN | NaN | 0.0054 | 0.0008 | 0.0042 |
| TBCB | NaN | NaN | NaN | NaN | NaN | NaN | 0.0342 | 0.0004 | 0.0071 |
| SMC5 | NaN | NaN | NaN | NaN | NaN | NaN | 0.0102 | 0.0007 | 0.0128 |
| KLHDC2 | NaN | NaN | NaN | NaN | NaN | NaN | 0.1743 | 0.0005 | 0.0009 |
| MPV17 | NaN | NaN | NaN | NaN | NaN | NaN | 0.0121 | 0.0001 | 0.007 |
| RBPJ | NaN | NaN | NaN | NaN | NaN | NaN | 0.0153 | 0.0008 | 0.0051 |
| POP5 | NaN | NaN | NaN | NaN | NaN | NaN | 0.0998 | 0.0008 | 0.0129 |
| PPAPDC1B | NaN | NaN | NaN | NaN | NaN | NaN | 0.025 | 0.0009 | 0.0115 |
| IMP3 | NaN | NaN | NaN | NaN | NaN | NaN | 0.1676 | 0.0014 | 0.012 |
| RNPS1 | 0.0006 | 0.0028 | 0.0052 | 0.0826 | 0.0228 | 0.0216 | NaN | NaN | NaN |
| NFE2L2 | NaN | NaN | NaN | 0.0513 | 0.0002 | 0.0119 | NaN | NaN | NaN |
| SOD1 | 0.0039 | 0.0765 | 0.0336 | 0.038 | 0.001 | 0.0017 | NaN | NaN | NaN |
| CD8B | 0.0001 | 0.064 | 0.0026 | NaN | NaN | NaN | 0.0786 | 0.1809 | 0.0184 |
| PTPN6 | 0.0001 | 0.0069 | 0.0015 | 0.0325 | 0.1521 | 0.0469 | 0.0343 | 0.0786 | 0.0219 |
| HSPA1B | 0.0001 | 0.5116 | 0.1942 | 0.0543 | 0.7989 | 0.4734 | 0.0693 | 0.0916 | 0.4033 |
| CD2BP2 | 0.0008 | 0.0002 | 0.0002 | 0.0317 | 0.2705 | 0.0067 | 0.1364 | 0.0586 | 0.2566 |
| ALDOA | 0 | 0.0018 | 0.0001 | NaN | NaN | NaN | NaN | NaN | NaN |
| ZFP36L1 | 0 | 0 | 0.0003 | 0.0065 | 0.1581 | 0.1141 | 0.013 | 0.3914 | 0.0687 |
| HSPB1 | 0 | 0 | 0 | NaN | NaN | NaN | 0.0522 | 0.1217 | 0.0224 |
| HSPA6 | 0.0005 | 0.1271 | 0.022 | NaN | NaN | NaN | NaN | NaN | NaN |
| ARHGEF1 | 0.0003 | 0.0616 | 0.0057 | NaN | NaN | NaN | NaN | NaN | NaN |
| LUC7L3 | 0 | 0 | 0.0002 | NaN | NaN | NaN | 0.2052 | 0.0253 | 0.0796 |
| GPR174 | 0.0006 | 0.0013 | 0.0006 | NaN | NaN | NaN | NaN | NaN | NaN |
| ENTPD1 | 0 | 0 | 0.0001 | NaN | NaN | NaN | NaN | NaN | NaN |
| RASSF5 | 0 | 0 | 0 | 0.3038 | 0.4883 | 0.0393 | NaN | NaN | NaN |
| IPCEF1 | 0 | 0.0019 | 0.0007 | NaN | NaN | NaN | NaN | NaN | NaN |
| ARNT | 0 | 0.0604 | 0.0005 | NaN | NaN | NaN | NaN | NaN | NaN |
| NAB1 | 0 | 0 | 0 | NaN | NaN | NaN | NaN | NaN | NaN |
| APLP2 | 0.0001 | 0.0996 | 0.021 | NaN | NaN | NaN | NaN | NaN | NaN |
| PRKCH | 0 | 0.0003 | 0.0002 | 0.0403 | 0.2771 | 0.0023 | NaN | NaN | NaN |
| SEMA4A | 0 | 0.0201 | 0.0019 | NaN | NaN | NaN | NaN | NaN | NaN |
| PPP1CC | 0.0003 | 0.0003 | 0 | NaN | NaN | NaN | NaN | NaN | NaN |
| LAG3 | 0 | 0.0058 | 0 | NaN | NaN | NaN | NaN | NaN | NaN |
| HSPA1A | 0 | 0.5279 | 0.0493 | 0.0356 | 0.8844 | 0.4747 | NaN | NaN | NaN |
| SNAP47 | 0 | 0 | 0 | 0.0036 | 0.1148 | 0.0998 | 0.0219 | 0.3669 | 0.5367 |
| CCL4L2 | 0.0008 | 0.0004 | 0.0003 | 0.0165 | 0.1728 | 0.0983 | NaN | NaN | NaN |
| ARID4B | 0 | 0 | 0 | 0.0071 | 0.2108 | 0.0664 | NaN | NaN | NaN |
| LYST | 0 | 0.0001 | 0 | NaN | NaN | NaN | NaN | NaN | NaN |
| NMB | 0 | 0.0028 | 0.0158 | NaN | NaN | NaN | NaN | NaN | NaN |
| LIMS1 | 0 | 0.0001 | 0.0001 | NaN | NaN | NaN | NaN | NaN | NaN |
| ITK | 0 | 0 | 0 | NaN | NaN | NaN | NaN | NaN | NaN |
| RILPL2 | 0.0001 | 0 | 0 | NaN | NaN | NaN | NaN | NaN | NaN |
| RGS3 | 0.0004 | 0.0004 | 0 | NaN | NaN | NaN | NaN | NaN | NaN |
| TRAT1 | 0 | 0.0001 | 0.0031 | 0.6107 | 0.0586 | 0.1847 | NaN | NaN | NaN |
| ELF1 | 0.0002 | 0.0164 | 0.0219 | NaN | NaN | NaN | NaN | NaN | NaN |
| OSBPL3 | 0 | 0.0017 | 0 | NaN | NaN | NaN | NaN | NaN | NaN |
| BIRC3 | 0 | 0.0638 | 0.002 | NaN | NaN | NaN | NaN | NaN | NaN |
| PTGER4 | 0.0004 | 0.0018 | 0.0022 | NaN | NaN | NaN | NaN | NaN | NaN |
| SERINC3 | 0.0003 | 0.0014 | 0.0038 | 0.0901 | 0.0045 | 0.0637 | NaN | NaN | NaN |
| MED7 | NaN | NaN | NaN | NaN | NaN | NaN | NaN | NaN | NaN |
| DDX3X | 0 | 0.0144 | 0.0011 | NaN | NaN | NaN | NaN | NaN | NaN |
| THEM6 | NaN | NaN | NaN | NaN | NaN | NaN | NaN | NaN | NaN |
| P4HA1 | NaN | NaN | NaN | NaN | NaN | NaN | NaN | NaN | NaN |
| HIBCH | NaN | NaN | NaN | NaN | NaN | NaN | NaN | NaN | NaN |
| VCAM1 | 0 | 0.0676 | 0.0006 | 0.0401 | 0.6271 | 0.3471 | NaN | NaN | NaN |
| FABP5 | 0 | 0.045 | 0.0009 | 0.2604 | 0.051 | 0.0716 | 0.0683 | 0.1498 | 0.2192 |
| NOL7 | NaN | NaN | NaN | 0.1273 | 0.0292 | 0.0075 | NaN | NaN | NaN |
| SEC14L1 | NaN | NaN | NaN | 0.0413 | 0.0566 | 0.0006 | NaN | NaN | NaN |
| UBA2 | NaN | NaN | NaN | NaN | NaN | NaN | NaN | NaN | NaN |
| CDCA4 | NaN | NaN | NaN | 0.374 | 0.0008 | 0.1195 | NaN | NaN | NaN |
| ATP5I | NaN | NaN | NaN | NaN | NaN | NaN | 0.1484 | 0.083 | 0.307 |
| ALKBH3 | NaN | NaN | NaN | NaN | NaN | NaN | NaN | NaN | NaN |
| DND1 | NaN | NaN | NaN | NaN | NaN | NaN | NaN | NaN | NaN |
| RNF185 | NaN | NaN | NaN | NaN | NaN | NaN | NaN | NaN | NaN |
| AFAP1L2 | NaN | NaN | NaN | NaN | NaN | NaN | NaN | NaN | NaN |
| GLOD4 | NaN | NaN | NaN | 0.0761 | 0.0123 | 0.0985 | NaN | NaN | NaN |
| PIP5K1A | NaN | NaN | NaN | NaN | NaN | NaN | NaN | NaN | NaN |
| ATF4 | NaN | NaN | NaN | NaN | NaN | NaN | 0.3136 | 0.026 | 0.1208 |
| PIGO | NaN | NaN | NaN | NaN | NaN | NaN | NaN | NaN | NaN |
| OPA1 | NaN | NaN | NaN | NaN | NaN | NaN | NaN | NaN | NaN |
| CCT3 | NaN | NaN | NaN | NaN | NaN | NaN | NaN | NaN | NaN |
| EXOSC6 | NaN | NaN | NaN | NaN | NaN | NaN | NaN | NaN | NaN |
| KIAA1429 | NaN | NaN | NaN | NaN | NaN | NaN | NaN | NaN | NaN |
| NDFIP2 | 0.0006 | 0.0122 | 0.0012 | NaN | NaN | NaN | NaN | NaN | NaN |
| TMEM222 | NaN | NaN | NaN | NaN | NaN | NaN | NaN | NaN | NaN |
| MYO1G | NaN | NaN | NaN | NaN | NaN | NaN | NaN | NaN | NaN |
| LBR | NaN | NaN | NaN | NaN | NaN | NaN | NaN | NaN | NaN |
| EXT2 | NaN | NaN | NaN | NaN | NaN | NaN | NaN | NaN | NaN |
| SARDH | NaN | NaN | NaN | NaN | NaN | NaN | 0.009 | 0.0547 | 0.0131 |
| POLR2I | NaN | NaN | NaN | NaN | NaN | NaN | NaN | NaN | NaN |
| HNRNPD | NaN | NaN | NaN | NaN | NaN | NaN | 0.063 | 0.0051 | 0.0159 |
| NAAA | NaN | NaN | NaN | NaN | NaN | NaN | NaN | NaN | NaN |
| ARID5A | NaN | NaN | NaN | NaN | NaN | NaN | NaN | NaN | NaN |
| PDRG1 | NaN | NaN | NaN | NaN | NaN | NaN | NaN | NaN | NaN |
| BCAP31 | NaN | NaN | NaN | NaN | NaN | NaN | 0.0783 | 0.0078 | 0.0446 |
| UQCRFS1 | NaN | NaN | NaN | NaN | NaN | NaN | 0.0454 | 0.0008 | 0.0886 |
| SNRNP40 | NaN | NaN | NaN | NaN | NaN | NaN | NaN | NaN | NaN |
| ASB8 | NaN | NaN | NaN | NaN | NaN | NaN | NaN | NaN | NaN |
| MRPL52 | NaN | NaN | NaN | NaN | NaN | NaN | NaN | NaN | NaN |
| TUG1 | NaN | NaN | NaN | NaN | NaN | NaN | NaN | NaN | NaN |
| CCND2 | NaN | NaN | NaN | NaN | NaN | NaN | NaN | NaN | NaN |
| NAA20 | NaN | NaN | NaN | NaN | NaN | NaN | NaN | NaN | NaN |
| HLA- | NaN | NaN | NaN | NaN | NaN | NaN | 0.0084 | 0.2112 | 0.285 |
| DPA1 | | | | | | | | | |
| TOX | 0 | 0.0079 | 0.0001 | NaN | NaN | NaN | 0.0232 | 0.2231 | 0.279 |
| TMEM205 | NaN | NaN | NaN | NaN | NaN | NaN | NaN | NaN | NaN |
| TPI1 | 0 | 0.0865 | 0 | NaN | NaN | NaN | 0.3128 | 0.0798 | 0.3281 |
| HADHA | 0.0004 | 0.0004 | 0.0054 | NaN | NaN | NaN | NaN | NaN | NaN |
| STAT3 | 0 | 0.0118 | 0.002 | NaN | NaN | NaN | 0.1821 | 0.0558 | 0.081 |
| GMDS | 0.0001 | 0.0126 | 0.0109 | NaN | NaN | NaN | NaN | NaN | NaN |
| SIRPG | 0.0005 | 0.0622 | 0.0005 | NaN | NaN | NaN | 0.1153 | 0.4323 | 0.1313 |
| ITM2A | 0 | 0 | 0 | 0.3873 | 0.3041 | 0.0132 | 0.0584 | 0.0129 | 0.0743 |
| TBC1D4 | 0 | 0.005 | 0.0001 | NaN | NaN | NaN | NaN | NaN | NaN |
| HNRNPM | 0.0001 | 0.0217 | 0 | NaN | NaN | NaN | NaN | NaN | NaN |
| ASB2 | 0 | 0.0008 | 0.0018 | 0.1221 | 0.0651 | 0.0184 | NaN | NaN | NaN |
| IGFLR1 | 0 | 0.0044 | 0 | 0.0034 | 0.1392 | 0.0647 | NaN | NaN | NaN |
| CD2 | 0.0003 | 0.0868 | 0.1255 | NaN | NaN | NaN | NaN | NaN | NaN |
| COTL1 | NaN | NaN | NaN | NaN | NaN | NaN | NaN | NaN | NaN |
| PBRM1 | NaN | NaN | NaN | NaN | NaN | NaN | NaN | NaN | NaN |
| DUT | NaN | NaN | NaN | 0.0742 | 0.0002 | 0.0048 | 0.1954 | 0.041 | 0.1202 |
| LMF2 | NaN | NaN | NaN | 0.0045 | 0.0005 | 0.0096 | 0.0563 | 0.0129 | 0.2073 |
| TAF15 | NaN | NaN | NaN | 0.0206 | 0.0003 | 0.0213 | NaN | NaN | NaN |
| H2AFY | NaN | NaN | NaN | 0.2419 | 0.0043 | 0.065 | 0.2576 | 0.0009 | 0.0021 |
| CEP57 | 0.0007 | 0.0185 | 0 | NaN | NaN | NaN | 0.0997 | 0.0286 | 0.1303 |
| AMDHD2 | NaN | NaN | NaN | NaN | NaN | NaN | NaN | NaN | NaN |
| SERINC1 | 0.0002 | 0.062 | 0.0623 | NaN | NaN | NaN | 0.0972 | 0.13 | 0.023 |
| CKS2 | 0.0009 | 0.1517 | 0.0033 | NaN | NaN | NaN | 0.0831 | 0.0031 | 0.1046 |
| PTPN11 | 0 | 0 | 0 | NaN | NaN | NaN | 0.0773 | 0.016 | 0.0282 |
| DDX3Y | 0.0001 | 0 | 0.0001 | NaN | NaN | NaN | NaN | NaN | NaN |
| IRF9 | 0 | 0.0064 | 0.0003 | NaN | NaN | NaN | 0.029 | 0.039 | 0.1103 |
| FYN | 0 | 0.0014 | 0.0004 | NaN | NaN | NaN | 0.0478 | 0.0808 | 0.1242 |
| HSPD1 | 0.0003 | 0.0017 | 0.0002 | 0.0712 | 0.2379 | 0.008 | 0.1798 | 0.0839 | 0.0612 |
| FPGS | 0 | 0 | 0.0005 | NaN | NaN | NaN | 0.7949 | 0.0251 | 0.2568 |
| CCT2 | 0.0003 | 0.0109 | 0.0019 | NaN | NaN | NaN | NaN | NaN | NaN |
| GNAS | 0.0009 | 0.0011 | 0.0004 | 0.05 | 0.0094 | 0.002 | NaN | NaN | NaN |
| FAIM3 | 0 | 0.0002 | 0 | NaN | NaN | NaN | NaN | NaN | NaN |
| ETV1 | 0 | 0.0002 | 0 | NaN | NaN | NaN | 0.0085 | 0.0638 | 0.0984 |
| BCL6 | 0.0001 | 0.0455 | 0.0076 | NaN | NaN | NaN | NaN | NaN | NaN |
| SLC38A1 | 0 | 0 | 0 | NaN | NaN | NaN | NaN | NaN | NaN |
| PDE7B | 0 | 0 | 0 | NaN | NaN | NaN | NaN | NaN | NaN |
| STAT1 | 0.0002 | 0.0003 | 0.0005 | NaN | NaN | NaN | NaN | NaN | NaN |
| EIF3H | 0.0002 | 0.0198 | 0.0063 | NaN | NaN | NaN | NaN | NaN | NaN |
| EID1 | 0 | 0 | 0 | NaN | NaN | NaN | 0.007 | 0.0252 | 0.0394 |
| ID3 | 0 | 0 | 0 | NaN | NaN | NaN | 0.1079 | 0.0348 | 0.0114 |
| PSAP | 0.0003 | 0.001 | 0 | NaN | NaN | NaN | 0.1474 | 0.1558 | 0.1225 |
| DPP7 | 0.0001 | 0.0005 | 0.0142 | NaN | NaN | NaN | 0.0934 | 0.0236 | 0.1165 |
| PJA2 | 0.0005 | 0.0009 | 0.0003 | NaN | NaN | NaN | 0.0582 | 0.0006 | 0.0024 |
| TARDBP | 0.0004 | 0.0001 | 0.0019 | NaN | NaN | NaN | 0.7056 | 0.0463 | 0.1404 |
| SRSF1 | 0.0002 | 0.119 | 0.0001 | NaN | NaN | NaN | NaN | NaN | NaN |
| GABPB1 | 0.0007 | 0.0048 | 0.0001 | NaN | NaN | NaN | NaN | NaN | NaN |
| RGS4 | 0 | 0.0001 | 0 | NaN | NaN | NaN | NaN | NaN | NaN |
| SPTAN1 | 0 | 0.0058 | 0.0012 | NaN | NaN | NaN | NaN | NaN | NaN |
| NFATC1 | 0 | 0 | 0 | NaN | NaN | NaN | NaN | NaN | NaN |
| HAVCR2 | 0 | 0 | 0 | NaN | NaN | NaN | NaN | NaN | NaN |
| PDCD1 | 0 | 0 | 0 | 0.0549 | 0.7301 | 0.2838 | NaN | NaN | NaN |
| SRSF4 | 0.0002 | 0.0489 | 0.0078 | NaN | NaN | NaN | NaN | NaN | NaN |
| GFOD1 | 0 | 0.0119 | 0 | NaN | NaN | NaN | NaN | NaN | NaN |
| MRPS21 | 0.0001 | 0.0104 | 0.0011 | NaN | NaN | NaN | NaN | NaN | NaN |
| AP3S1 | 0.0008 | 0 | 0.0002 | NaN | NaN | NaN | NaN | NaN | NaN |
| GPBP1 | 0.0003 | 0.0087 | 0.0025 | NaN | NaN | NaN | NaN | NaN | NaN |
| BTLA | 0 | 0.001 | 0 | 0.1127 | 0.1656 | 0.0095 | NaN | NaN | NaN |
| PAM | 0 | 0.0042 | 0.0003 | NaN | NaN | NaN | NaN | NaN | NaN |
| CBLB | 0 | 0.0173 | 0 | 0 | 0.0382 | 0.0014 | NaN | NaN | NaN |
| ATHL1 | 0 | 0.0003 | 0.0001 | 0 | 0.0003 | 0.1186 | NaN | NaN | NaN |
| MGEA5 | 0 | 0 | 0.0001 | 0.0001 | 0.0077 | 0.019 | NaN | NaN | NaN |
| IRF4 | 0.0002 | 0.0003 | 0 | 0.0116 | 0.0058 | 0.0202 | NaN | NaN | NaN |
| UBE2F | 0.0002 | 0.0009 | 0.0001 | NaN | NaN | NaN | NaN | NaN | NaN |
| SFXN1 | 0 | 0.0003 | 0 | NaN | NaN | NaN | NaN | NaN | NaN |
| DGKH | 0 | 0 | 0 | 0.0429 | 0.1319 | 0.0048 | NaN | NaN | NaN |
| FCRL3 | 0 | 0 | 0 | 0.0038 | 0.4334 | 0.1878 | NaN | NaN | NaN |
| PYHIN1 | 0.0005 | 0.237 | 0.0725 | NaN | NaN | NaN | NaN | NaN | NaN |
| EIF1B | 0.0005 | 0.0274 | 0.0237 | NaN | NaN | NaN | NaN | NaN | NaN |
| RAPGEF6 | 0 | 0.0071 | 0.0006 | 0.0066 | 0.312 | 0.0243 | NaN | NaN | NaN |
| SNX9 | 0 | 0.0003 | 0 | 0 | 0.0822 | 0.018 | NaN | NaN | NaN |
| IL6ST | 0 | 0.0003 | 0 | 0.001 | 0.3163 | 0.1457 | NaN | NaN | NaN |
| PTPN7 | 0 | 0.0001 | 0.0001 | 0.0187 | 0.0692 | 0.0099 | 0.0562 | 0.2593 | 0.4005 |
| CREM | 0.0001 | 0.0002 | 0.0001 | NaN | NaN | NaN | NaN | NaN | NaN |
| HNRPLL | 0 | 0 | 0 | NaN | NaN | NaN | NaN | NaN | NaN |
| FUT8 | 0 | 0 | 0 | NaN | NaN | NaN | NaN | NaN | NaN |
| LITAF | 0 | 0 | 0.0011 | 0.0174 | 0.0699 | 0.0064 | NaN | NaN | NaN |
| TSC22D1 | 0.0003 | 0.0417 | 0.0072 | 0.0064 | 0.2037 | 0.0921 | NaN | NaN | NaN |
| TRAF5 | 0 | 0.0004 | 0 | 0.0017 | 0.0029 | 0.0041 | 0.0191 | 0.5496 | 0.3241 |
| ATP6V0B | 0.0007 | 0.0004 | 0.007 | 0.245 | 0.0209 | 0.2399 | NaN | NaN | NaN |
| SRSF6 | 0.001 | 0.0025 | 0.0001 | NaN | NaN | NaN | NaN | NaN | NaN |
| ELMO1 | 0 | 0.0001 | 0.0051 | NaN | NaN | NaN | NaN | NaN | NaN |
| IRF8 | 0 | 0 | 0 | NaN | NaN | NaN | NaN | NaN | NaN |
| TAGAP | 0.0004 | 0.0035 | 0 | NaN | NaN | NaN | NaN | NaN | NaN |
| CADM1 | 0 | 0.0022 | 0 | NaN | NaN | NaN | NaN | NaN | NaN |
| SPRY2 | 0 | 0.0029 | 0.0179 | NaN | NaN | NaN | NaN | NaN | NaN |
| CTLA4 | 0 | 0 | 0 | 0.0197 | 0.0112 | 0.044 | NaN | NaN | NaN |
| ANKRD10 | 0 | 0.0799 | 0.0824 | NaN | NaN | NaN | NaN | NaN | NaN |
| KLRK1 | 0.0001 | 0.2117 | 0.1051 | NaN | NaN | NaN | NaN | NaN | NaN |
| TP53INP1 | 0 | 0.0301 | 0.0005 | 0.0052 | 0.2705 | 0.1118 | NaN | NaN | NaN |
| NR4A2 | 0.0008 | 0.0027 | 0.0002 | 0.0349 | 0.3516 | 0.3808 | NaN | NaN | NaN |
| ZNF292 | 0 | 0.0001 | 0 | NaN | NaN | NaN | NaN | NaN | NaN |
| MIF4GD | 0.0004 | 0 | 0 | NaN | NaN | NaN | NaN | NaN | NaN |
| ING3 | NaN | NaN | NaN | 0.2464 | 0.0001 | 0.0067 | NaN | NaN | NaN |
| SQSTM1 | NaN | NaN | NaN | 0.0418 | 0.0007 | 0.0012 | NaN | NaN | NaN |
| CLK4 | NaN | NaN | NaN | 0.1075 | 0.0002 | 0.0703 | NaN | NaN | NaN |
| NCBP2 | NaN | NaN | NaN | 0.1415 | 0 | 0.0145 | NaN | NaN | NaN |
| SET | NaN | NaN | NaN | 0.3262 | 0.0005 | 0.0514 | NaN | NaN | NaN |
| PSME3 | NaN | NaN | NaN | 0.0097 | 0.001 | 0.0047 | NaN | NaN | NaN |
| IQCB1 | NaN | NaN | NaN | 0.0048 | 0.0001 | 0.0035 | NaN | NaN | NaN |
| RGCC | NaN | NaN | NaN | 0.005 | 0 | 0 | NaN | NaN | NaN |
| C20orf111 | NaN | NaN | NaN | 0.1713 | 0.0003 | 0.0008 | NaN | NaN | NaN |
| MPP1 | NaN | NaN | NaN | 0.0019 | 0.0004 | 0.0007 | NaN | NaN | NaN |
| CALR | NaN | NaN | NaN | 0.032 | 0 | 0.0002 | NaN | NaN | NaN |
| TMEM160 | NaN | NaN | NaN | 0.303 | 0.0006 | 0.0306 | NaN | NaN | NaN |
| SRGN | 0 | 0.0201 | 0.0018 | 0.0001 | 0.0023 | 0.0015 | 0.0091 | 0.0731 | 0.1072 |
| EWSR1 | 0.0007 | 0.7415 | 0.4508 | 0.0624 | 0.1121 | 0.0025 | NaN | NaN | NaN |
| EZR | 0.0003 | 0.0002 | 0.0284 | 0.003 | 0.0062 | 0.0016 | NaN | NaN | NaN |
| FTSJ3 | NaN | NaN | NaN | 0.0149 | 0.0001 | 0.0036 | NaN | NaN | NaN |
| LRMP | 0 | 0.0947 | 0.0257 | NaN | NaN | NaN | NaN | NaN | NaN |
| GBP2 | 0 | 0 | 0 | 0.0007 | 0.0001 | 0.0152 | 0.0194 | 0.0595 | 0.0663 |
| MPG | 0.0006 | 0.0408 | 0.105 | NaN | NaN | NaN | NaN | NaN | NaN |
| RELA | NaN | NaN | NaN | 0.0497 | 0.0002 | 0.0043 | NaN | NaN | NaN |
| KLHDC4 | NaN | NaN | NaN | 0.0524 | 0 | 0.0092 | NaN | NaN | NaN |
| PMS2P1 | NaN | NaN | NaN | 0.0009 | 0.0003 | 0.0022 | NaN | NaN | NaN |
| CWF19L1 | NaN | NaN | NaN | 0.0035 | 0 | 0.0001 | NaN | NaN | NaN |
| AP2S1 | NaN | NaN | NaN | 0.0109 | 0.0003 | 0.0007 | NaN | NaN | NaN |
| RAE1 | NaN | NaN | NaN | 0.0676 | 0.0006 | 0.1764 | NaN | NaN | NaN |
| TRIP12 | NaN | NaN | NaN | 0.0427 | 0.0002 | 0.0254 | 0.0009 | 0.0756 | 0.045 |
| PDZD11 | NaN | NaN | NaN | 0.5152 | 0.0009 | 0.2748 | NaN | NaN | NaN |
| SPG21 | NaN | NaN | NaN | 0.0031 | 0 | 0.0002 | NaN | NaN | NaN |
| RRM1 | NaN | NaN | NaN | 0.2638 | 0.0003 | 0.1623 | NaN | NaN | NaN |
| SUB1 | NaN | NaN | NaN | 0.0175 | 0.0006 | 0.0059 | NaN | NaN | NaN |
| RAB11F1P1 | NaN | NaN | NaN | 0.022 | 0.0004 | 0.0052 | NaN | NaN | NaN |
| USO1 | NaN | NaN | NaN | 0.0007 | 0 | 0 | NaN | NaN | NaN |
| NIPSNAP3A | NaN | NaN | NaN | 0.0915 | 0 | 0 | NaN | NaN | NaN |
| ANAPC13 | NaN | NaN | NaN | 0.0059 | 0.0009 | 0.0029 | NaN | NaN | NaN |
| AEN | NaN | NaN | NaN | 0.0178 | 0 | 0 | NaN | NaN | NaN |
| SF3B4 | NaN | NaN | NaN | 0.0158 | 0.0001 | 0.0124 | NaN | NaN | NaN |
| CAV1 | NaN | NaN | NaN | 0.0348 | 0.0001 | 0.0036 | NaN | NaN | NaN |
| PSPC1 | NaN | NaN | NaN | 0.1295 | 0.0002 | 0.0018 | NaN | NaN | NaN |
| TFRC | NaN | NaN | NaN | 0.0433 | 0.0007 | 0.0026 | NaN | NaN | NaN |
| WDR48 | NaN | NaN | NaN | 0.0324 | 0.0001 | 0.0082 | NaN | NaN | NaN |
| INO80C | NaN | NaN | NaN | 0.0442 | 0 | 0.0099 | NaN | NaN | NaN |
| NOP58 | 0.0002 | 0.0011 | 0.0005 | 0.0001 | 0.0014 | 0.0156 | NaN | NaN | NaN |
| NFAT5 | NaN | NaN | NaN | 0 | 0.001 | 0.0071 | NaN | NaN | NaN |
| LBH | 0.0004 | 0.0313 | 0.0018 | 0.0012 | 0.0013 | 0.0101 | NaN | NaN | NaN |
| LMAN2 | NaN | NaN | NaN | 0.0008 | 0.0007 | 0.0025 | NaN | NaN | NaN |
| ACOT9 | NaN | NaN | NaN | 0.01 | 0 | 0.0093 | NaN | NaN | NaN |
| BRAP | NaN | NaN | NaN | 0.009 | 0 | 0 | NaN | NaN | NaN |
| SLC7A5 | 0.001 | 0.0008 | 0 | 0.1968 | 0.0001 | 0.0016 | NaN | NaN | NaN |
| CCT5 | NaN | NaN | NaN | 0.1323 | 0.0005 | 0.049 | NaN | NaN | NaN |
| NAT10 | NaN | NaN | NaN | 0.1519 | 0 | 0.0063 | NaN | NaN | NaN |
| YBX1 | NaN | NaN | NaN | 0.2843 | 0 | 0.0006 | NaN | NaN | NaN |
| IMPDH2 | NaN | NaN | NaN | 0.0644 | 0.0001 | 0 | NaN | NaN | NaN |
| PPM1B | NaN | NaN | NaN | 0.0486 | 0 | 0.0004 | NaN | NaN | NaN |
| BANF1 | NaN | NaN | NaN | 0.0574 | 0.0005 | 0.061 | NaN | NaN | NaN |
| PLEKHO2 | NaN | NaN | NaN | 0.0093 | 0.0003 | 0.0004 | NaN | NaN | NaN |
| HSPBP1 | NaN | NaN | NaN | 0.0006 | 0 | 0 | NaN | NaN | NaN |
| JTB | NaN | NaN | NaN | 0.0755 | 0.001 | 0.0262 | NaN | NaN | NaN |
| SRA1 | NaN | NaN | NaN | 0.0219 | 0.0002 | 0.0003 | NaN | NaN | NaN |
| METTL9 | NaN | NaN | NaN | 0.0361 | 0.0001 | 0.0085 | NaN | NaN | NaN |
| SLC44A2 | NaN | NaN | NaN | 0.0184 | 0.0314 | 0.0066 | NaN | NaN | NaN |
| MYCBP | NaN | NaN | NaN | 0.0173 | 0 | 0.0003 | NaN | NaN | NaN |
| KIAA0101 | NaN | NaN | NaN | 0.5451 | 0 | 0.068 | NaN | NaN | NaN |

| P-values from comparison | |
|---|---|
| of high vs. low exhaustion cells in each tumor | |
| mel74 p-value | mel58 p-value |

| | | | tumor/ | | | tumor/ |
|---|---|---|---|---|---|---|
| Gene | Mel75 | viral | circulation | Mel75 | viral | circulation |
| Names | program | (Wherry) | (Baitch) | program | (Wherry) | (Baitch) |
| Consistent | | | | | | |
| across | | | | | | |
| tumors | | | | | | |
| (FIG. 5E) | | | | | | |
| CXCL13 | 0 | 0 | 0 | 0.0005 | 0.0028 | 0.002 |
| TNFRSF1B | 0.0022 | 0.3017 | 0.1745 | 0.0099 | 0.0059 | 0.0217 |
| RGS2 | 0.002 | 0.1416 | 0.0188 | 0.1229 | 0.2167 | 0.3588 |
| TIGIT | 0 | 0.0018 | 0.0176 | 0.0908 | 0.4075 | 0.5944 |
| CD27 | 0.0002 | 0.0098 | 0.0018 | 0.0036 | 0.0334 | 0.0164 |
| TNFRSF9 | 0 | 0.0135 | 0 | 0.0005 | 0.0054 | 0.0135 |
| SLA | 0.0004 | 0.004 | 0.0008 | NaN | NaN | NaN |
| RNF19A | 0.001 | 0.0091 | 0.0034 | 0.0203 | 0.0247 | 0.1226 |
| INPP5F | 0.0006 | 0.0011 | 0.0003 | 0.1131 | 0.1163 | 0.0505 |
| XCL2 | 0.0161 | 0.3603 | 0.1345 | 0.2351 | 0.0313 | 0.003 |
| HLA- | 0 | 0.0291 | 0.0058 | 0.0265 | 0.24 | 0.0623 |
| DMA | | | | | | |
| FAM3C | 0.0019 | 0.0035 | 0.0127 | 0.0359 | 0.131 | 0.3485 |
| UQCRC1 | 0.0246 | 0.0272 | 0.0342 | 0.0691 | 0.1336 | 0.0437 |
| WARS | NaN | NaN | NaN | 0.0005 | 0.0339 | 0.0071 |
| EIF3L | 0.0136 | 0.0289 | 0.0499 | NaN | NaN | NaN |
| KCNK5 | 0.0022 | 0.003 | 0.0014 | 0.2479 | 0.0315 | 0.0976 |
| TMBIM6 | 0.0245 | 0.386 | 0.0298 | 0.4653 | 0.1742 | 0.4908 |
| CD200 | 0.0309 | 0.0813 | 0.0427 | 0.0003 | 0.0057 | 0.2413 |
| ZC3H7A | 0.0804 | 0.0196 | 0.0147 | 0.0479 | 0.006 | 0.0313 |
| SH2D1A | 0.0017 | 0.004 | 0.0125 | NaN | NaN | NaN |
| ATP1B3 | 0 | 0.0008 | 0.0005 | NaN | NaN | NaN |
| MYO7A | 0.0072 | 0.2347 | 0.0121 | 0.0319 | 0.0372 | 0.0073 |
| THADA | 0.0001 | 0.001 | 0.0001 | NaN | NaN | NaN |
| PARK7 | NaN | NaN | NaN | NaN | NaN | NaN |
| EGR2 | 0.0121 | 0.0239 | 0.0022 | 0.0122 | 0.0042 | 0.0122 |
| FDFT1 | 0.0217 | 0.012 | 0.031 | NaN | NaN | NaN |
| CRTAM | 0.0009 | 0.1845 | 0.0244 | NaN | NaN | NaN |
| IFI16 | NaN | NaN | NaN | 0.1386 | 0.0404 | 0.0469 |
| variable | | | | | | |
| across | | | | | | |
| tumors | | | | | | |
| (FIG. 5F) | | | | | | |
| GMNN | NaN | NaN | NaN | NaN | NaN | NaN |
| AFG3L1P | NaN | NaN | NaN | NaN | NaN | NaN |
| CSRP1 | NaN | NaN | NaN | NaN | NaN | NaN |
| RBM5 | NaN | NaN | NaN | 0.2732 | 0.2895 | 0.0357 |
| AP1M1 | NaN | NaN | NaN | NaN | NaN | NaN |
| NUCB2 | NaN | NaN | NaN | 0.1269 | 0.0241 | 0.0548 |
| NOP10 | NaN | NaN | NaN | 0.0676 | 0.0427 | 0.2486 |
| GFM1 | NaN | NaN | NaN | 0.0443 | 0.2515 | 0.1701 |
| DHRS7 | NaN | NaN | NaN | NaN | NaN | NaN |
| SSU72 | NaN | NaN | NaN | NaN | NaN | NaN |
| SBDS | NaN | NaN | NaN | NaN | NaN | NaN |
| ATP6V1B2 | NaN | NaN | NaN | NaN | NaN | NaN |
| VAPA | NaN | NaN | NaN | NaN | NaN | NaN |
| CSNK2A1 | NaN | NaN | NaN | NaN | NaN | NaN |
| LINC00339 | NaN | NaN | NaN | NaN | NaN | NaN |
| MRPL4 | 0.1891 | 0.3017 | 0.0154 | NaN | NaN | NaN |
| PPP1R2 | 0.0773 | 0.102 | 0.2452 | NaN | NaN | NaN |
| SMG1 | NaN | NaN | NaN | NaN | NaN | NaN |
| OIP5- | NaN | NaN | NaN | NaN | NaN | NaN |
| AS1 | | | | | | |
| LPAR2 | NaN | NaN | NaN | NaN | NaN | NaN |
| LSMD1 | NaN | NaN | NaN | NaN | NaN | NaN |
| STAG3L4 | NaN | NaN | NaN | NaN | NaN | NaN |
| P4HB | NaN | NaN | NaN | NaN | NaN | NaN |
| SKP1 | 0.1019 | 0.5039 | 0.029 | NaN | NaN | NaN |
| PTBP1 | 0.2127 | 0.0279 | 0.1136 | NaN | NaN | NaN |
| TSTA3 | 0.018 | 0.0068 | 0.0749 | NaN | NaN | NaN |
| TBCB | 0.0629 | 0.0132 | 0.0012 | NaN | NaN | NaN |
| SMC5 | NaN | NaN | NaN | NaN | NaN | NaN |
| KLHDC2 | 0.1559 | 0.0344 | 0.1269 | NaN | NaN | NaN |
| MPV17 | 0.0083 | 0.0553 | 0.1799 | NaN | NaN | NaN |
| RBPJ | 0.0494 | 0.1263 | 0.0397 | NaN | NaN | NaN |
| POP5 | 0.0473 | 0.2087 | 0.1016 | NaN | NaN | NaN |
| PPAPDC1B | NaN | NaN | NaN | NaN | NaN | NaN |
| IMP3 | 0.0019 | 0 | 0.0005 | NaN | NaN | NaN |
| RNPS1 | 0.2514 | 0.005 | 0.1041 | NaN | NaN | NaN |
| NFE2L2 | 0.2721 | 0.0159 | 0.1404 | NaN | NaN | NaN |
| SOD1 | 0.0218 | 0.028 | 0.0652 | NaN | NaN | NaN |
| CD8B | 0.1733 | 0.0553 | 0.4022 | NaN | NaN | NaN |
| PTPN6 | 0.0041 | 0.3226 | 0.1166 | NaN | NaN | NaN |
| HSPA1B | 0.0004 | 0.108 | 0.0022 | NaN | NaN | NaN |
| CD2BP2 | 0.0236 | 0.095 | 0.0196 | NaN | NaN | NaN |
| ALDOA | 0.1106 | 0.1592 | 0.2918 | NaN | NaN | NaN |
| ZFP36L1 | 0.0467 | 0.133 | 0.0186 | NaN | NaN | NaN |
| HSPB1 | 0 | 0.0033 | 0.0001 | NaN | NaN | NaN |
| HSPA6 | 0.067 | 0.2844 | 0.0681 | NaN | NaN | NaN |
| ARHGEF1 | 0.1164 | 0.2448 | 0.0685 | NaN | NaN | NaN |
| LUC7L3 | 0.0217 | 0.0497 | 0.071 | NaN | NaN | NaN |
| GPR174 | NaN | NaN | NaN | NaN | NaN | NaN |
| ENTPD1 | 0.0016 | 0.0284 | 0.0383 | NaN | NaN | NaN |
| RASSF5 | 0.0181 | 0.0726 | 0.0085 | NaN | NaN | NaN |
| IPCEF1 | 0.1529 | 0.0325 | 0.0074 | NaN | NaN | NaN |
| ARNT | NaN | NaN | NaN | NaN | NaN | NaN |
| NAB1 | 0.0014 | 0.0429 | 0.0021 | NaN | NaN | NaN |
| APLP2 | 0.0479 | 0.182 | 0.0864 | NaN | NaN | NaN |
| PRKCH | 0.003 | 0.0102 | 0.0137 | NaN | NaN | NaN |
| SEMA4A | 0.0046 | 0.0573 | 0.0578 | NaN | NaN | NaN |
| PPP1CC | 0.0054 | 0.0032 | 0 | NaN | NaN | NaN |
| LAG3 | 0.0083 | 0.0124 | 0.0071 | NaN | NaN | NaN |
| HSPA1A | 0 | 0.0014 | 0.0005 | NaN | NaN | NaN |
| SNAP47 | 0 | 0.0005 | 0.0022 | NaN | NaN | NaN |
| CCL4L2 | 0.0003 | 0.0004 | 0.0021 | NaN | NaN | NaN |
| ARID4B | 0.0096 | 0.0332 | 0.0078 | NaN | NaN | NaN |
| LYST | 0.0004 | 0.0385 | 0.0256 | NaN | NaN | NaN |
| NMB | 0.0074 | 0.0264 | 0.0528 | NaN | NaN | NaN |
| LIMS1 | 0.0276 | 0.0015 | 0.0197 | NaN | NaN | NaN |
| ITK | 0.0207 | 0.0021 | 0.002 | NaN | NaN | NaN |
| RILPL2 | 0.0123 | 0.0166 | 0.0274 | NaN | NaN | NaN |
| RGS3 | 0.1051 | 0.0088 | 0.1025 | 0.1165 | 0.7017 | 0.1923 |
| TRAT1 | 0.0222 | 0.0627 | 0.0728 | NaN | NaN | NaN |
| ELF1 | NaN | NaN | NaN | NaN | NaN | NaN |
| OSBPL3 | 0.0047 | 0.0834 | 0.0224 | NaN | NaN | NaN |
| BIRC3 | 0.0588 | 0.4373 | 0.1928 | NaN | NaN | NaN |
| PTGER4 | NaN | NaN | NaN | NaN | NaN | NaN |
| SERINC3 | 0.0088 | 0.0105 | 0.0932 | NaN | NaN | NaN |
| MED7 | 0.1987 | 0.0211 | 0 | NaN | NaN | NaN |
| DDX3X | 0.049 | 0.0007 | 0 | NaN | NaN | NaN |
| THEM6 | 0.0353 | 0.0177 | 0.001 | NaN | NaN | NaN |
| P4HA1 | 0.1085 | 0.0035 | 0.0006 | 0.059 | 0.1071 | 0.059 |
| HIBCH | 0.0187 | 0.0141 | 0 | NaN | NaN | NaN |
| VCAM1 | 0.0001 | 0.01 | 0.0016 | 0.0428 | 0.0878 | 0.1046 |
| FABP5 | 0 | 0.057 | 0.0385 | 0.043 | 0.4296 | 0.0971 |
| NOL7 | 0.0081 | 0.0002 | 0.001 | NaN | NaN | NaN |
| SEC14L1 | 0.0029 | 0.0005 | 0.0001 | NaN | NaN | NaN |
| UBA2 | 0.0212 | 0.001 | 0.001 | NaN | NaN | NaN |
| CDCA4 | 0.0037 | 0.0477 | 0.0477 | NaN | NaN | NaN |
| ATP5I | 0.0725 | 0.0444 | 0.0007 | NaN | NaN | NaN |
| ALKBH3 | 0.0079 | 0 | 0 | NaN | NaN | NaN |
| DND1 | 0.0054 | 0.0016 | 0.0008 | NaN | NaN | NaN |
| RNF185 | 0.0243 | 0.0002 | 0 | NaN | NaN | NaN |
| AFAP1L2 | 0.006 | 0.0014 | 0.0006 | NaN | NaN | NaN |
| GLOD4 | 0.0005 | 0.0089 | 0 | NaN | NaN | NaN |
| PIP5K1A | 0.0012 | 0.0002 | 0.0001 | NaN | NaN | NaN |
| ATF4 | 0.0017 | 0.0019 | 0.0004 | NaN | NaN | NaN |
| PIGO | 0.0021 | 0 | 0 | NaN | NaN | NaN |
| OPA1 | 0.0006 | 0.0006 | 0.0004 | NaN | NaN | NaN |
| CCT3 | 0.0003 | 0 | 0 | NaN | NaN | NaN |
| EXOSC6 | 0.0071 | 0 | 0.0001 | NaN | NaN | NaN |
| KIAA1429 | 0.0685 | 0.0013 | 0 | NaN | NaN | NaN |
| NDFIP2 | 0.0029 | 0.0055 | 0.0006 | NaN | NaN | NaN |
| TMEM222 | 0.04 | 0.1649 | 0.0007 | NaN | NaN | NaN |
| MYO1G | 0.0015 | 0.0001 | 0 | NaN | NaN | NaN |
| LBR | 0.0167 | 0.0078 | 0.0004 | NaN | NaN | NaN |
| EXT2 | 0.0004 | 0 | 0 | NaN | NaN | NaN |
| SARDH | 0.0142 | 0.0363 | 0.0009 | NaN | NaN | NaN |
| POLR2I | 0.0453 | 0.0051 | 0 | NaN | NaN | NaN |
| HNRNPD | 0.0137 | 0 | 0 | NaN | NaN | NaN |
| NAAA | 0.0113 | 0.0046 | 0.0008 | NaN | NaN | NaN |
| ARID5A | 0.002 | 0.0007 | 0.0004 | 0.1861 | 0.3083 | 0.1303 |
| PDRG1 | 0.0108 | 0.0008 | 0.0008 | NaN | NaN | NaN |
| BCAP31 | 0.0017 | 0.0058 | 0.0005 | 0.0854 | 0.0802 | 0.2539 |
| UQCRFS1 | 0.0121 | 0.0216 | 0.0027 | 0.0596 | 0.0743 | 0.0596 |
| SNRNP40 | 0.0292 | 0.0597 | 0.0005 | 0.0112 | 0.0428 | 0.0397 |
| ASB8 | 0.0302 | 0.0008 | 0.0003 | 0.0297 | 0.0167 | 0.0065 |
| MRPL52 | 0.0535 | 0.0019 | 0.0002 | 0.0489 | 0.0867 | 0.0353 |
| TUG1 | 0.0445 | 0.0135 | 0.0006 | 0.084 | 0.0378 | 0.0277 |
| CCND2 | 0.0092 | 0.0042 | 0.0003 | 0.0859 | 0.018 | 0.156 |
| NAA20 | 0.0001 | 0 | 0 | 0.0185 | 0.1032 | 0.1284 |
| HLA- | 0 | 0.0014 | 0 | 0.0127 | 0.0809 | 0.0345 |
| DPA1 | | | | | | |
| TOX | 0 | 0 | 0 | 0.0561 | 0.0702 | 0.0026 |
| TMEM205 | 0.0567 | 0.0134 | 0 | 0.1357 | 0.0294 | 0.1357 |
| TPI1 | 0.043 | 0.3116 | 0.092 | 0.0361 | 0.066 | 0.1803 |
| HADHA | 0.0539 | 0.0523 | 0.0308 | 0.0194 | 0.1972 | 0.0135 |
| STAT3 | 0.0914 | 0.0247 | 0.3064 | 0.073 | 0.0621 | 0.0105 |
| GMDS | NaN | NaN | NaN | 0.0072 | 0.0108 | 0.0091 |
| SIRPG | NaN | NaN | NaN | 0.0329 | 0.021 | 0.0004 |
| ITM2A | 0.0863 | 0.4551 | 0.0868 | 0.0424 | 0.0636 | 0.0005 |
| TBC1D4 | NaN | NaN | NaN | 0.0093 | 0.004 | 0.048 |
| HNRNPM | NaN | NaN | NaN | NaN | NaN | NaN |
| ASB2 | NaN | NaN | NaN | 0.0301 | 0.1408 | 0.0413 |
| IGFLR1 | 0.0577 | 0.7887 | 0.4566 | 0.0091 | 0.0064 | 0.0032 |
| CD2 | NaN | NaN | NaN | 0.0734 | 0.0089 | 0.159 |
| COTL1 | NaN | NaN | NaN | 0.0002 | 0.0001 | 0.0113 |
| PBRM1 | NaN | NaN | NaN | 0.0038 | 0.0004 | 0.0699 |
| DUT | NaN | NaN | NaN | 0.0375 | 0.1219 | 0.0069 |
| LMF2 | NaN | NaN | NaN | 0.1372 | 0.2222 | 0.0042 |
| TAF15 | NaN | NaN | NaN | NaN | NaN | NaN |
| H2AFY | NaN | NaN | NaN | NaN | NaN | NaN |
| CEP57 | NaN | NaN | NaN | NaN | NaN | NaN |
| AMDHD2 | NaN | NaN | NaN | 0.1346 | 0 | 0.3301 |
| SERINC1 | NaN | NaN | NaN | 0.164 | 0.1954 | 0.0893 |
| CKS2 | NaN | NaN | NaN | 0.0515 | 0.4373 | 0.0047 |
| PTPN11 | NaN | NaN | NaN | 0.0589 | 0.2881 | 0.1291 |
| DDX3Y | NaN | NaN | NaN | NaN | NaN | NaN |
| IRF9 | NaN | NaN | NaN | NaN | NaN | NaN |
| FYN | NaN | NaN | NaN | NaN | NaN | NaN |
| HSPD1 | NaN | NaN | NaN | NaN | NaN | NaN |
| FPGS | NaN | NaN | NaN | NaN | NaN | NaN |
| CCT2 | NaN | NaN | NaN | NaN | NaN | NaN |
| GNAS | 0.0282 | 0.6765 | 0.5578 | NaN | NaN | NaN |
| FAIM3 | NaN | NaN | NaN | NaN | NaN | NaN |
| ETV1 | NaN | NaN | NaN | NaN | NaN | NaN |
| BCL6 | NaN | NaN | NaN | NaN | NaN | NaN |
| SLC38A1 | NaN | NaN | NaN | NaN | NaN | NaN |
| PDE7B | NaN | NaN | NaN | NaN | NaN | NaN |
| STAT1 | NaN | NaN | NaN | NaN | NaN | NaN |
| EIF3H | NaN | NaN | NaN | 0.2083 | 0.787 | 0.7731 |
| EID1 | NaN | NaN | NaN | NaN | NaN | NaN |
| ID3 | 0.0613 | 0.7721 | 0.4407 | NaN | NaN | NaN |
| PSAP | NaN | NaN | NaN | NaN | NaN | NaN |
| DPP7 | NaN | NaN | NaN | NaN | NaN | NaN |
| PJA2 | NaN | NaN | NaN | NaN | NaN | NaN |
| TARDBP | NaN | NaN | NaN | 0.1352 | 0.5102 | 0.6772 |
| SRSF1 | NaN | NaN | NaN | NaN | NaN | NaN |
| GABPB1 | NaN | NaN | NaN | NaN | NaN | NaN |
| RGS4 | NaN | NaN | NaN | 0.0137 | 0.1013 | 0.2991 |
| SPTAN1 | NaN | NaN | NaN | NaN | NaN | NaN |
| NFATC1 | NaN | NaN | NaN | NaN | NaN | NaN |
| HAVCR2 | 0.0097 | 0.1294 | 0.0222 | 0.1262 | 0.4065 | 0.1166 |
| PDCD1 | 0.0007 | 0.0344 | 0.0265 | 0.0354 | 0.1134 | 0.1379 |
| SRSF4 | NaN | NaN | NaN | 0.083 | 0.0281 | 0.0346 |
| GFOD1 | NaN | NaN | NaN | 0.0303 | 0.0418 | 0.0054 |
| MRPS21 | 0.2322 | 0.2293 | 0.1042 | 0.2373 | 0.1692 | 0.026 |
| AP3S1 | NaN | NaN | NaN | NaN | NaN | NaN |
| GPBP1 | NaN | NaN | NaN | NaN | NaN | NaN |
| BTLA | NaN | NaN | NaN | NaN | NaN | NaN |
| PAM | 0.0262 | 0.0402 | 0.0391 | NaN | NaN | NaN |
| CBLB | 0.002 | 0.2094 | 0.0644 | 0.0147 | 0.0634 | 0.0623 |
| ATHL1 | 0.0115 | 0.131 | 0.1105 | 0.0463 | 0.1952 | 0.0544 |
| MGEA5 | NaN | NaN | NaN | 0.1082 | 0.0707 | 0.1219 |
| IRF4 | NaN | NaN | NaN | 0.0381 | 0.0965 | 0.0238 |
| UBE2F | NaN | NaN | NaN | 0.2342 | 0.2807 | 0.091 |
| SFXN1 | NaN | NaN | NaN | 0.2483 | 0.1632 | 0.104 |
| DGKH | NaN | NaN | NaN | NaN | NaN | NaN |
| FCRL3 | NaN | NaN | NaN | 0.1685 | 0.227 | 0.0521 |
| PYHIN1 | NaN | NaN | NaN | NaN | NaN | NaN |
| EIF1B | NaN | NaN | NaN | NaN | NaN | NaN |
| RAPGEF6 | NaN | NaN | NaN | NaN | NaN | NaN |
| SNX9 | NaN | NaN | NaN | NaN | NaN | NaN |
| IL6ST | NaN | NaN | NaN | NaN | NaN | NaN |
| PTPN7 | 0.0998 | 0.9452 | 0.5431 | NaN | NaN | NaN |
| CREM | NaN | NaN | NaN | NaN | NaN | NaN |
| HNRPLL | NaN | NaN | NaN | NaN | NaN | NaN |
| FUT8 | NaN | NaN | NaN | NaN | NaN | NaN |
| LITAF | NaN | NaN | NaN | NaN | NaN | NaN |
| TSC22D1 | NaN | NaN | NaN | NaN | NaN | NaN |
| TRAF5 | NaN | NaN | NaN | NaN | NaN | NaN |
| ATP6V0B | NaN | NaN | NaN | NaN | NaN | NaN |
| SRSF6 | NaN | NaN | NaN | NaN | NaN | NaN |
| ELMO1 | NaN | NaN | NaN | NaN | NaN | NaN |
| IRF8 | NaN | NaN | NaN | NaN | NaN | NaN |
| TAGAP | NaN | NaN | NaN | NaN | NaN | NaN |
| CADM1 | NaN | NaN | NaN | NaN | NaN | NaN |
| SPRY2 | NaN | NaN | NaN | NaN | NaN | NaN |
| CTLA4 | 0.0585 | 0.3935 | 0.2015 | NaN | NaN | NaN |
| ANKRD10 | NaN | NaN | NaN | NaN | NaN | NaN |
| KLRK1 | NaN | NaN | NaN | NaN | NaN | NaN |
| TP53INP1 | NaN | NaN | NaN | NaN | NaN | NaN |
| NR4A2 | 0.0729 | 0.5481 | 0.2769 | NaN | NaN | NaN |
| ZNF292 | NaN | NaN | NaN | NaN | NaN | NaN |
| MIF4GD | NaN | NaN | NaN | NaN | NaN | NaN |
| ING3 | NaN | NaN | NaN | NaN | NaN | NaN |
| SQSTM1 | NaN | NaN | NaN | NaN | NaN | NaN |
| CLK4 | NaN | NaN | NaN | NaN | NaN | NaN |
| NCBP2 | NaN | NaN | NaN | NaN | NaN | NaN |
| SET | NaN | NaN | NaN | 0.2632 | 0.0758 | 0.3145 |
| PSME3 | NaN | NaN | NaN | NaN | NaN | NaN |
| IQCB1 | NaN | NaN | NaN | NaN | NaN | NaN |
| RGCC | NaN | NaN | NaN | NaN | NaN | NaN |
| C20orf111 | NaN | NaN | NaN | NaN | NaN | NaN |
| MPP1 | NaN | NaN | NaN | NaN | NaN | NaN |
| CALR | NaN | NaN | NaN | 0.155 | 0.5841 | 0.3465 |
| TMEM160 | NaN | NaN | NaN | 0.044 | 0.6557 | 0.4359 |
| SRGN | 0.0021 | 0.32 | 0.1818 | 0.0097 | 0.0646 | 0.2565 |
| EWSR1 | NaN | NaN | NaN | 0.1059 | 0.5566 | 0.1467 |
| EZR | NaN | NaN | NaN | 0.3427 | 0.3071 | 0.1584 |
| FTSJ3 | NaN | NaN | NaN | NaN | NaN | NaN |
| LRMP | NaN | NaN | NaN | NaN | NaN | NaN |
| GBP2 | NaN | NaN | NaN | 0.0163 | 0.1335 | 0.011 |
| MPG | NaN | NaN | NaN | 0.2952 | 0.0908 | 0.3179 |
| RELA | NaN | NaN | NaN | NaN | NaN | NaN |
| KLHDC4 | NaN | NaN | NaN | NaN | NaN | NaN |
| PMS2P1 | NaN | NaN | NaN | NaN | NaN | NaN |
| CWF19L1 | NaN | NaN | NaN | NaN | NaN | NaN |
| AP2S1 | NaN | NaN | NaN | NaN | NaN | NaN |
| RAE1 | NaN | NaN | NaN | NaN | NaN | NaN |
| TRIP12 | NaN | NaN | NaN | NaN | NaN | NaN |
| PDZD11 | NaN | NaN | NaN | NaN | NaN | NaN |
| SPG21 | NaN | NaN | NaN | NaN | NaN | NaN |
| RRM1 | NaN | NaN | NaN | NaN | NaN | NaN |
| SUB1 | NaN | NaN | NaN | NaN | NaN | NaN |
| RAB11F1P1 | NaN | NaN | NaN | NaN | NaN | NaN |
| USO1 | NaN | NaN | NaN | NaN | NaN | NaN |
| NIPSNAP3A | NaN | NaN | NaN | NaN | NaN | NaN |
| ANAPC13 | NaN | NaN | NaN | NaN | NaN | NaN |
| AEN | NaN | NaN | NaN | NaN | NaN | NaN |
| SF3B4 | NaN | NaN | NaN | NaN | NaN | NaN |
| CAV1 | NaN | NaN | NaN | NaN | NaN | NaN |
| PSPC1 | NaN | NaN | NaN | NaN | NaN | NaN |
| TFRC | NaN | NaN | NaN | NaN | NaN | NaN |
| WDR48 | NaN | NaN | NaN | NaN | NaN | NaN |
| INO80C | NaN | NaN | NaN | NaN | NaN | NaN |
| NOP58 | NaN | NaN | NaN | NaN | NaN | NaN |
| NFAT5 | NaN | NaN | NaN | NaN | NaN | NaN |
| LBH | 0.1347 | 0.0534 | 0.0779 | 0.1179 | 0.3594 | 0.6117 |
| LMAN2 | 0.1643 | 0.04 | 0.046 | 0.0961 | 0.1512 | 0.6309 |
| ACOT9 | NaN | NaN | NaN | NaN | NaN | NaN |
| BRAP | NaN | NaN | NaN | NaN | NaN | NaN |
| SLC7A5 | 0.0017 | 0.0277 | 0.11 | 0.2015 | 0.4307 | 0.0179 |
| CCT5 | NaN | NaN | NaN | NaN | NaN | NaN |
| NAT10 | NaN | NaN | NaN | NaN | NaN | NaN |
| YBX1 | NaN | NaN | NaN | NaN | NaN | NaN |
| IMPDH2 | NaN | NaN | NaN | NaN | NaN | NaN |
| PPM1B | NaN | NaN | NaN | NaN | NaN | NaN |
| BANF1 | 0.1081 | 0.1981 | 0.0489 | NaN | NaN | NaN |
| PLEKHO2 | 0.0603 | 0.0369 | 0.022 | NaN | NaN | NaN |
| HSPBP1 | 0.0421 | 0.0746 | 0.0002 | NaN | NaN | NaN |
| JTB | 0.0898 | 0.0003 | 0.0125 | NaN | NaN | NaN |
| SRA1 | 0.0185 | 0.0062 | 0.0529 | NaN | NaN | NaN |
| METTL9 | 0.0223 | 0.0881 | 0.0645 | NaN | NaN | NaN |
| SLC44A2 | 0.0833 | 0.0017 | 0.0002 | 0.0399 | 0.0812 | 0.0116 |
| MYCBP | NaN | NaN | NaN | NaN | NaN | NaN |
| KIAA0101 | NaN | NaN | NaN | 0.1779 | 0.1909 | 0.0379 |

| | P-values from comparison of each tumor to all other tumors (sign indicates | | | | | | | |
|---|---|---|---|---|---|---|---|---|
| | direction of change) | | | | | | | |
| | mel75 p-value | | | mel79 p-value | | | mel89 p-value | |
| | | | tumor/ | | | tumor/ | | |
| Gene | Mel75 | viral | circulation | Mel75 | viral | circulation | Mel75 | viral |
| Names | program | (Wherry) | (Baitch) | program | (Wherry) | (Baitch) | program | (Wherry) |
| Consistent | | | | | | | | |
| across | | | | | | | | |
| tumors | | | | | | | | |
| (FIG. 5E) | | | | | | | | |
| CXCL13 | −0.2288 | −0.0123 | −0.1156 | −0.0015 | −1.00E−04 | −0.0047 | 0.0117 | 0.2438 |
| TNFRSF1B | 0.0503 | −0.3508 | 0.246 | 0.0592 | 0.0743 | 0.0935 | 0.2254 | −0.3462 |
| RGS2 | 0.0006 | 0.0345 | 0.0022 | −0.4114 | −0.0841 | −0.0638 | −0.2902 | 0.3219 |
| TIGIT | 0.0466 | 0.2647 | 0.3767 | −0.3642 | −0.2978 | −0.4099 | −0.0034 | −0.0875 |
| CD27 | 0.0323 | −0.4739 | 0.1598 | −0.0094 | −1.00E−04 | −1.00E−04 | −0.0002 | −0.0004 |
| TNFRSF9 | 0.01 | 0.1654 | 0.0542 | −0.2915 | −0.0159 | −0.0029 | −1.00E−04 | −1.00E−04 |
| SLA | 0.2011 | −0.3995 | 0.4704 | 0.126 | −0.4069 | −0.4973 | 0.2266 | −0.2764 |
| RNF19A | 0.0002 | 0.0011 | 0.0022 | −0.4692 | −0.0455 | −0.1378 | −0.0847 | −0.0163 |
| INPP5F | 0.0529 | 0.1044 | 0.1027 | −0.1374 | −0.1935 | −0.176 | −0.0817 | −0.0077 |
| XCL2 | −0.3423 | −0.1705 | −0.2119 | −0.459 | 0.2313 | 0.1 | −0.1827 | 0.4017 |
| HLA- | 0.3125 | −0.1654 | −0.4192 | −0.2673 | −0.4148 | −0.0672 | −0.0164 | −0.0102 |
| DMA | | | | | | | | |
| FAM3C | 0.327 | 0.4062 | 0.4317 | 0.1971 | 0.3269 | −0.2668 | −0.0097 | −0.0065 |
| UQCRC1 | −0.2955 | −0.2291 | −0.2332 | 0.1858 | 0.005 | 0.0447 | −0.2941 | 0.0602 |
| WARS | 0.1808 | 0.4479 | 0.2814 | 0.1186 | 0.0866 | 0.1091 | −1.00E−04 | −0.0007 |
| EIF3L | −0.2867 | −0.4003 | −0.4288 | −0.0605 | −0.4301 | −0.1162 | −0.0409 | 0.0435 |
| KCNK5 | 0.0672 | 0.3582 | 0.4393 | −0.0562 | −0.1526 | −0.3586 | 0.1207 | 0.3078 |
| TMB1M6 | 0.2937 | −0.2026 | −0.488 | 0.0776 | 0.2671 | 0.0614 | −0.4857 | −0.3101 |
| CD200 | 0.0225 | 0.2502 | 0.3734 | 0.1723 | −0.2488 | −0.361 | −0.0009 | −0.0004 |
| ZC3H7A | 0.0443 | 0.1407 | 0.2467 | −0.1249 | −0.0287 | −0.0327 | −0.0791 | −0.0504 |
| SH2D1A | 0.3087 | −0.4329 | −0.3759 | −0.0071 | −0.0477 | −0.0397 | 0.2019 | 0.0049 |
| ATP1B3 | −0.4595 | −0.205 | −0.4071 | −0.0139 | −0.3562 | −0.1769 | −0.0036 | −0.0196 |
| MYO7A | −0.0336 | −0.0321 | −0.112 | −0.0627 | −1.00E−04 | −0.042 | 0.1005 | 0.045 |
| THADA | 0.0005 | 0.0408 | 0.0082 | 0.4745 | −0.4207 | −0.4363 | −0.2535 | 0.3542 |
| PARK7 | 0.1856 | 0.0625 | 0.0856 | −0.0599 | 0.2651 | −0.2468 | −0.236 | 0.2149 |
| EGR2 | 0.1081 | 0.2466 | 0.2395 | −0.3918 | 0.2234 | −0.4516 | −0.0005 | −1.00E−04 |
| FDFT1 | 0.052 | 0.0928 | 0.0815 | −0.353 | 0.2317 | 0.1648 | −0.0364 | −0.0512 |
| CRTAM | 0.2633 | −0.381 | 0.1792 | 0.4742 | 0.2242 | 0.0496 | −0.1556 | −0.0753 |
| IFI16 | 0.0116 | 0.0547 | 0.0672 | −0.2839 | −0.0207 | −0.2284 | 0.002 | 0.0056 |
| variable | | | | | | | | |
| across | | | | | | | | |
| tumors | | | | | | | | |
| (FIG. 5F) | | | | | | | | |
| GMNN | −0.2289 | −0.3561 | −0.3291 | −0.0974 | −0.3842 | −0.2104 | −0.1481 | 0.0001 |
| AFG3L1P | −0.0417 | −0.1272 | −0.1039 | −0.292 | −0.0151 | −0.1581 | 0.0446 | 0.0001 |
| CSRP1 | −0.3608 | −0.4007 | −0.1615 | −0.0101 | −0.0003 | −0.3445 | 0.0044 | 0.0001 |
| RBM5 | −0.0071 | −0.0649 | −0.0217 | −0.215 | −0.002 | −0.0571 | 0.0169 | 0.0004 |
| AP1M1 | −0.0097 | −0.0003 | −0.0005 | −0.0413 | −0.0206 | −0.0282 | 0.0142 | 0.0001 |
| NUCB2 | 0.3412 | −0.1776 | −0.1095 | −0.4192 | 0.244 | −0.4558 | 0.0227 | 0.0019 |
| NOP10 | 0.486 | 0.2565 | −0.4459 | −0.3124 | 0.4829 | −0.2973 | 0.1049 | 0.0001 |
| GFM1 | −0.0755 | −0.0911 | −0.1491 | −0.1669 | −0.0689 | −0.2417 | −0.3254 | 0.056 |
| DHRS7 | −0.3057 | −0.3779 | −0.282 | 0.3167 | −0.1792 | −0.4523 | 0.0277 | 0.0001 |
| SSU72 | 0.4922 | −0.4556 | −0.3902 | −0.0127 | −0.0156 | −0.0573 | 0.0002 | 0.0001 |
| SBDS | −0.4688 | −0.3298 | −0.3303 | −0.2381 | 0.4351 | −0.1562 | 0.0162 | 0.0003 |
| ATP6V1B2 | 0.1827 | 0.2851 | 0.4224 | −0.3525 | 0.0666 | 0.0258 | 0.3606 | 0.0016 |
| VAPA | 0.3796 | −0.1173 | −0.3373 | −0.4961 | 0.0998 | 0.1076 | 0.0033 | 0.0002 |
| CSNK2A1 | 0.4061 | 0.1749 | 0.3593 | 0.4523 | 0.1568 | 0.3153 | 0.2716 | 0.0017 |
| LINC00339 | −0.2824 | −0.2652 | −0.4698 | 0.1412 | 0.192 | 0.4537 | 0.0673 | 0.0004 |
| MRPL4 | −0.0124 | −0.0034 | −0.0014 | 0.1402 | 0.1337 | −0.4394 | 0.2589 | 0.0037 |
| PPP1R2 | −0.1421 | −0.001 | −0.0817 | −0.1822 | 0.1781 | 0.1634 | 0.2458 | 0.1153 |
| SMG1 | −0.3799 | −0.0424 | −0.0913 | 0.0164 | 0.0746 | 0.0433 | 0.0266 | 0.0003 |
| OIP5- | −0.0204 | −0.2388 | −0.0419 | 0.0622 | 0.0145 | 0.0064 | 0.012 | 0.0023 |
| AS1 | | | | | | | | |
| LPAR2 | 0.1736 | 0.2355 | 0.2021 | 0.182 | 0.0472 | 0.0482 | 0.0055 | 0.0001 |
| LSMD1 | −0.0772 | −0.0691 | −0.0474 | −0.3252 | −0.3162 | −0.1623 | 0.0004 | 0.0001 |
| STAG3L4 | −0.3457 | −0.4334 | −0.4687 | −0.2149 | −0.3401 | −0.1553 | 0.0057 | 0.0002 |
| P4HB | −0.4159 | 0.1266 | −0.4327 | 0.4951 | 0.1844 | 0.2581 | 0.0406 | 0.0001 |
| SKP1 | −0.1295 | −0.0661 | −0.0609 | −0.1247 | −0.0514 | −0.1349 | 0.0479 | 0.007 |
| PTBP1 | −0.1399 | −0.1136 | −0.2903 | −0.14 | −0.3412 | −0.3575 | 0.0614 | 0.0025 |
| TSTA3 | −0.0693 | −0.2543 | −0.0728 | 0.4518 | −0.4266 | 0.2787 | 0.0001 | 0.0001 |
| TBCB | −0.0452 | −0.223 | −0.101 | −0.0367 | −0.4601 | −0.3659 | 0.0056 | 0.0001 |
| SMC5 | −0.0071 | −1.00E−04 | −1.00E−04 | −0.0017 | −0.3589 | −0.009 | 0.0498 | 0.0031 |
| KLHDC2 | −0.2556 | −0.4518 | −0.2413 | −0.0659 | −0.1541 | −0.0755 | 0.4876 | 0.0204 |
| MPV17 | −0.1968 | −0.253 | −0.1358 | −0.3003 | −0.0701 | −0.0374 | 0.0017 | 0.0001 |
| RBPJ | −0.2516 | −0.1551 | −0.2562 | −0.0364 | −0.0168 | −0.0229 | 0.0258 | 0.0005 |
| POP5 | 0.3099 | 0.4566 | 0.4173 | −0.3799 | −0.1645 | −0.0556 | 0.2215 | 0.0066 |
| PPAPDC1B | 0.2372 | 0.1666 | 0.0907 | 0.4113 | −0.0003 | −0.0615 | 0.046 | 0.0024 |
| IMP3 | −0.3594 | −0.2217 | −0.3826 | −0.0097 | −0.0104 | −0.0797 | −0.3837 | 0.012 |
| RNPS1 | 0.0152 | 0.0452 | 0.057 | 0.1032 | 0.0332 | 0.0308 | −0.4511 | 0.1957 |
| NFE2L2 | 0.2762 | 0.2463 | 0.1474 | 0.024 | 0.0001 | 0.004 | −0.1029 | 0.4159 |
| SOD1 | −0.2012 | −0.0525 | −0.0857 | −0.2787 | 0.2611 | 0.3047 | −0.095 | −0.1876 |
| CD8B | 0.1592 | −0.4287 | 0.2738 | −0.3675 | −0.3132 | −0.3528 | 0.2498 | 0.4485 |
| PTPN6 | 0.2166 | 0.4417 | 0.3301 | 0.4843 | −0.2477 | −0.4471 | 0.1929 | 0.3566 |
| HSPA1B | 0.0007 | −0.4965 | 0.2217 | 0.0729 | −0.0756 | −0.3504 | 0.0411 | 0.0628 |
| CD2BP2 | 0.3881 | 0.3269 | 0.3094 | 0.4754 | −0.111 | 0.2391 | −0.4117 | 0.3302 |
| ALDOA | 0.116 | 0.3051 | 0.1685 | −0.1551 | −0.1349 | −0.3586 | 0.3169 | −0.3873 |
| ZFP36L1 | 0.204 | 0.4327 | 0.4571 | 0.4148 | −0.076 | −0.1193 | 0.0298 | −0.1139 |
| HSPB1 | 0.0549 | 0.2416 | 0.053 | −0.0686 | −1.00E−04 | −1.00E−04 | −0.4441 | −0.227 |
| HSPA6 | 0.0035 | 0.1546 | 0.0418 | −0.048 | −1.00E−04 | −1.00E−04 | −0.419 | 0.3714 |
| ARHGEF1 | 0.1316 | 0.4856 | 0.2835 | −0.1593 | −0.0264 | −0.1384 | −0.1263 | −0.2435 |
| LUC7L3 | 0.0565 | 0.0423 | 0.1468 | −0.0143 | −0.0359 | −0.0686 | −0.4585 | 0.083 |
| GPR174 | 0.0018 | 0.0029 | 0.0016 | −0.0082 | −1.00E−04 | −0.0278 | −0.1908 | 0.1945 |
| ENTPD1 | 0.0011 | 0.0001 | 0.0054 | −0.1602 | −0.0502 | −0.0373 | 0.2765 | 0.4783 |
| RASSF5 | 0.1784 | 0.2674 | 0.2585 | −0.0345 | −0.009 | −0.2675 | −0.1244 | −0.292 |
| IPCEF1 | 0.0936 | 0.3209 | 0.2853 | −0.0447 | −0.0129 | −0.0406 | −0.2604 | −0.2049 |
| ARNT | 0.003 | 0.2292 | 0.0219 | −0.0228 | −0.0706 | −0.0896 | −0.2949 | −0.3174 |
| NAB1 | 0.0019 | 0.028 | 0.0321 | 0.4281 | −0.0602 | −0.4658 | −0.1741 | −0.2929 |
| APLP2 | 0.0107 | 0.2326 | 0.1093 | 0.1777 | 0.4921 | 0.2999 | −0.0925 | −0.0329 |
| PRKCH | 0.044 | 0.424 | 0.3348 | −0.2685 | −0.038 | 0.3414 | −0.0471 | −0.0027 |
| SEMA4A | 0.003 | 0.0923 | 0.0295 | 0.2385 | 0.2412 | −0.4482 | −0.1127 | −0.1437 |
| PPP1CC | 0.2126 | 0.2113 | 0.1073 | −0.1336 | −0.1509 | −0.1165 | −0.0007 | −0.0008 |
| LAG3 | 0.0223 | 0.2548 | 0.0769 | −0.267 | −0.152 | −0.273 | −0.2747 | −0.2306 |
| HSPA1A | 0.0008 | −0.487 | 0.0842 | 0.0418 | −0.0446 | −0.3966 | −0.449 | −0.1634 |
| SNAP47 | 0.2904 | −0.4134 | 0.4122 | −0.3289 | −0.0341 | −0.0421 | 0.483 | −0.0088 |
| CCL4L2 | −0.3814 | −0.4287 | −0.446 | −0.339 | −0.0629 | −0.1123 | −0.0704 | −0.0013 |
| ARID4B | 0.0328 | 0.1452 | 0.0381 | 0.0735 | −0.4116 | 0.3001 | −0.4809 | −0.0619 |
| LYST | 0.0001 | 0.02 | 0.0013 | −0.4729 | −0.0141 | −0.2837 | −0.2584 | −0.0011 |
| NMB | 0.0006 | 0.1022 | 0.2272 | −0.3172 | 0.3715 | −0.362 | −0.2415 | −0.0842 |
| LIMS1 | 0.0051 | 0.0929 | 0.0939 | 0.3909 | 0.2007 | −0.3781 | 0.34 | −0.4533 |
| ITK | 0.0003 | 0.0003 | 0.0002 | 0.3752 | 0.2329 | −0.4769 | −0.3938 | 0.4021 |
| RILPL2 | 0.0921 | 0.0479 | 0.0235 | −0.4759 | −0.4788 | 0.2426 | −0.1604 | −0.4893 |
| RGS3 | 0.142 | 0.1426 | 0.0426 | −0.2125 | −0.4115 | −0.2344 | −0.0806 | −0.122 |
| TRAT1 | 0.0197 | 0.0482 | 0.2086 | −0.0446 | 0.4578 | −0.2954 | −0.0034 | −0.0159 |
| ELF1 | 0.0582 | 0.1998 | 0.2211 | −0.1133 | −0.1364 | −0.0588 | −0.0752 | −0.0059 |
| OSBPL3 | 0.0963 | −0.4347 | 0.3285 | −0.1109 | −0.1593 | −0.0867 | −0.0148 | −0.0241 |
| BIRC3 | 0.0184 | 0.2999 | 0.0877 | −0.2503 | −0.3036 | −0.2134 | −0.1839 | −0.0503 |
| PTGER4 | 0.0052 | 0.0096 | 0.0109 | 0.3152 | 0.246 | −0.4519 | −0.0238 | −0.0068 |
| SERINC3 | 0.0052 | 0.0154 | 0.0264 | 0.0894 | 0.0015 | 0.0614 | −0.0023 | −1.00E−04 |
| MED7 | −0.4992 | 0.3021 | 0.4695 | −0.3103 | −0.2359 | 0.4724 | −0.0053 | −1.00E−04 |
| DDX3X | 0.1272 | 0.4972 | 0.2799 | −0.3843 | −0.0745 | −0.0423 | −0.006 | −0.0027 |
| THEM6 | −0.1169 | 0.4393 | 0.4442 | −0.3501 | −0.0283 | −0.3339 | −1.00E−04 | −1.00E−04 |
| P4HA1 | −0.2842 | −0.0578 | −0.1429 | −0.1656 | −0.1316 | −0.3016 | −0.1498 | −0.0007 |
| HIBCH | 0.3691 | 0.3816 | −0.4102 | −0.1157 | −0.0019 | −0.0104 | −0.0013 | −1.00E−04 |
| VCAM1 | 0.3379 | −0.1212 | −0.4253 | −0.2598 | −0.0023 | −0.0181 | −0.1353 | −0.0004 |
| FABP5 | 0.4124 | −0.1433 | −0.4141 | −0.0708 | −0.3087 | −0.25 | −0.2966 | −0.1478 |
| NOL7 | −0.2036 | −0.085 | −0.0641 | 0.2896 | 0.073 | 0.0252 | −0.0552 | −0.0447 |
| SEC14L1 | −0.0687 | −0.012 | −0.0839 | 0.1501 | 0.196 | 0.0048 | 0.3971 | 0.3578 |
| UBA2 | −0.0151 | −0.1556 | −0.0948 | −0.3426 | 0.1105 | 0.0409 | −0.338 | 0.3731 |
| CDCA4 | −0.0244 | −0.1091 | −0.1361 | −0.2945 | 0.0436 | 0.4314 | 0.2772 | 0.177 |
| ATP5I | −0.0207 | −0.0194 | −0.0019 | −0.1992 | −0.4899 | −0.1292 | 0.41 | 0.2607 |
| ALKBH3 | −1.00E−04 | −1.00E−04 | −1.00E−04 | −0.0372 | −0.0334 | −0.0069 | −0.131 | −0.1622 |
| DND1 | −0.05 | −0.0758 | −0.0158 | −0.1593 | −0.0808 | −0.0755 | 0.2508 | −0.2072 |
| RNF185 | −0.0861 | −0.1481 | −0.0101 | −0.0262 | −0.0379 | −0.075 | 0.3024 | −0.4216 |
| AFAP1L2 | −0.061 | −0.0026 | −0.0074 | −0.1176 | −0.1275 | −0.3083 | −0.3525 | −0.1578 |
| GLOD4 | −0.1416 | −0.0561 | −0.0388 | −0.3637 | 0.3351 | −0.3123 | 0.3694 | 0.4031 |
| PIP5K1A | −0.0084 | −0.0308 | −0.0758 | −0.172 | 0.3377 | −0.4541 | −0.0899 | −0.4456 |
| ATF4 | −0.3125 | −0.2254 | 0.2805 | 0.4699 | 0.3536 | 0.3765 | 0.4141 | 0.0209 |
| PIGO | −0.3749 | −0.0767 | −0.2085 | −0.4111 | −0.1275 | −0.176 | −0.0275 | −0.0484 |
| OPA1 | −0.2271 | −0.2216 | −0.3644 | −0.3177 | 0.4075 | −0.1257 | −0.0581 | −0.0834 |
| CCT3 | −0.2821 | −0.2534 | −0.0521 | −0.1467 | −0.1418 | −0.0221 | −0.0326 | −0.1264 |
| EXOSC6 | −0.1277 | −0.0735 | −0.0972 | −0.1798 | −0.0027 | −0.0739 | −0.3198 | 0.4582 |
| KIAA1429 | −0.2654 | −0.1234 | −0.4243 | −0.0691 | −0.2708 | −0.3621 | −0.3045 | −0.3784 |
| NDFIP2 | −0.2777 | −0.1049 | −0.2167 | −0.004 | −0.0141 | −0.1362 | −0.0108 | −0.1197 |
| TMEM222 | −0.3607 | −0.3926 | 0.2759 | −0.1798 | −0.3051 | −0.3822 | −0.0805 | 0.1899 |
| MYO1G | −0.1326 | −0.3676 | −0.1008 | −0.005 | −0.0911 | −0.0445 | −0.4784 | −0.3149 |
| LBR | −0.0034 | −0.002 | −0.0002 | −0.0251 | −0.126 | −0.3519 | −0.2889 | −0.4558 |
| EXT2 | −0.4621 | −0.1487 | −0.3899 | −0.0698 | −0.225 | 0.1797 | −0.3731 | 0.1721 |
| SARDH | −0.2453 | −0.1666 | −0.2083 | −0.0182 | −0.0047 | −0.0892 | −0.3588 | −0.162 |
| POLR2I | 0.2794 | 0.2282 | 0.2668 | −0.0002 | −0.0005 | −1.00E−04 | −0.4316 | 0.4161 |
| HNRNPD | −0.1578 | −0.1081 | −0.3288 | −0.0008 | −1.00E−04 | −0.0007 | −0.4225 | 0.0992 |
| NAAA | −0.1653 | −0.0234 | −0.0582 | −0.0127 | −1.00E−04 | −0.0015 | −0.0341 | −0.0346 |
| ARID5A | 0.3884 | −0.2573 | 0.2809 | −0.0014 | −0.0005 | −0.0022 | 0.44 | −0.247 |
| PDRG1 | −0.081 | −0.1119 | −0.0385 | −0.0016 | −0.0002 | −0.0002 | 0.435 | 0.3764 |
| BCAP31 | −0.0911 | −0.0505 | −0.1365 | −0.0057 | −0.0088 | −1.00E−04 | 0.2353 | 0.0199 |
| UQCRFS1 | −0.2463 | −0.4788 | 0.4416 | −0.0125 | −0.1927 | −0.1077 | 0.2165 | 0.0135 |
| SNRNP40 | −0.2798 | −0.1117 | −0.2127 | −0.1536 | −0.103 | −0.0448 | −0.4841 | 0.1147 |
| ASB8 | −0.0073 | −0.0016 | −0.0055 | −0.1458 | −0.0739 | −0.108 | −0.0141 | −0.158 |
| MRPL52 | −0.2388 | −0.4158 | −0.3681 | −0.3183 | −0.2667 | −0.0211 | −0.2073 | −0.2436 |
| TUG1 | −0.2853 | −0.418 | −0.3051 | −0.0378 | −0.2841 | −0.4161 | −0.1611 | −0.3838 |
| CCND2 | −0.0318 | −0.1267 | −0.168 | −0.0238 | −0.0418 | −0.0345 | −0.0441 | −0.2136 |
| NAA20 | −0.0018 | −0.0059 | −0.0015 | −1.00E−04 | −1.00E−04 | −0.0039 | −0.0186 | −0.0275 |
| HLA- | −0.4126 | −0.1581 | −0.2706 | −0.0588 | −0.0893 | −0.1585 | 0.0376 | −0.4379 |
| DPA1 | | | | | | | | |
| TOX | −0.4898 | −0.0301 | −0.1423 | −0.0002 | −1.00E−04 | −0.0006 | −0.1004 | −0.0035 |
| TMEM205 | 0.3455 | 0.3704 | 0.0829 | −0.1161 | −0.036 | −0.186 | −0.0127 | −0.3551 |
| TPI1 | 0.2905 | −0.2219 | 0.2925 | −0.0118 | −0.0311 | −0.1234 | −0.0906 | −0.4389 |
| HADHA | 0.075 | 0.064 | 0.1812 | −1.00E−04 | −0.0008 | −0.0005 | −0.0583 | −0.214 |
| STAT3 | 0.0135 | 0.1455 | 0.0768 | 0.4852 | −0.1051 | −0.3737 | 0.4405 | 0.1656 |
| GMDS | −0.4402 | −0.1674 | −0.1765 | −0.0017 | −1.00E−04 | −1.00E−04 | −0.0098 | −0.004 |
| SIRPG | 0.4301 | −0.205 | 0.4116 | −1.00E−04 | −0.003 | −0.0673 | −0.3164 | −0.039 |
| ITM2A | 0.0321 | 0.3921 | 0.2405 | −0.0061 | −0.0105 | −0.1741 | −0.1875 | −0.4512 |
| TBC1D4 | 0.0001 | 0.166 | 0.0208 | −0.0368 | −0.071 | −0.0232 | −0.3214 | 0.4892 |
| HNRNPM | 0.0124 | 0.0873 | 0.0081 | −0.0313 | −0.1139 | 0.4699 | −0.1541 | −0.2077 |
| ASB2 | 0.0388 | 0.111 | 0.1357 | 0.4571 | 0.3194 | 0.1473 | −0.1711 | 0.2396 |
| IGFLR1 | 0.0679 | −0.1957 | 0.2355 | −0.3871 | −0.0305 | −0.0766 | −0.0169 | −0.0169 |
| CD2 | 0.1588 | −0.3052 | −0.2539 | 0.4753 | −0.0366 | −0.0916 | −0.0046 | −0.0613 |
| COTL1 | 0.3083 | 0.4865 | −0.4624 | −0.0352 | −0.0051 | −0.3539 | −0.0212 | −0.0649 |
| PBRM1 | −0.0196 | −0.0189 | −0.0141 | −0.1516 | −0.104 | −0.4017 | −0.0663 | −0.2611 |
| DOT | −0.2032 | −0.3943 | −0.3383 | 0.466 | 0.0465 | 0.1439 | −0.475 | 0.1212 |
| LMF2 | −0.3221 | −0.1974 | −0.0742 | 0.0173 | 0.0018 | 0.0286 | 0.134 | 0.0215 |
| TAF15 | −0.0883 | −0.2132 | −0.0425 | 0.2126 | 0.0095 | 0.2164 | −0.3734 | 0.4705 |
| H2AFY | −0.1797 | −0.0965 | 0.4959 | −0.2497 | 0.0804 | 0.4166 | −0.3096 | 0.0044 |
| CEP57 | 0.3032 | −0.4262 | 0.1991 | −0.4412 | 0.4613 | −0.4677 | 0.3502 | 0.1216 |
| AMDHD2 | −0.3982 | −0.4668 | 0.1966 | 0.1187 | −0.4184 | 0.407 | 0.0018 | 0.0628 |
| SERINC1 | 0.0643 | 0.2875 | 0.2881 | −0.3982 | −0.4776 | −0.2826 | 0.0895 | 0.127 |
| CKS2 | 0.1566 | −0.3352 | 0.2515 | 0.2325 | −0.3138 | 0.4721 | 0.3022 | 0.0291 |
| PTPN11 | 0.0913 | 0.1332 | 0.1374 | −0.4527 | −0.0691 | −0.1562 | 0.3766 | 0.1169 |
| DDX3Y | 0.0029 | 0.0018 | 0.0041 | −0.3488 | −0.2526 | −0.2336 | 0.2267 | 0.092 |
| IRF9 | 0.0198 | 0.1737 | 0.0641 | −0.2147 | −0.0505 | −0.1077 | 0.0918 | 0.1165 |
| FYN | 0.0039 | 0.0363 | 0.0256 | −0.1091 | −0.1116 | −0.3257 | 0.0087 | 0.0222 |
| HSPD1 | −0.4898 | −0.3967 | 0.4279 | −0.2739 | −0.0956 | 0.3709 | −0.1828 | −0.385 |
| FPGS | 0.0066 | 0.0166 | 0.0437 | 0.3523 | −0.4548 | 0.1031 | −0.0151 | 0.0443 |
| CCT2 | 0.0196 | 0.0857 | 0.0384 | 0.3332 | 0.3523 | 0.1415 | −0.1157 | 0.1543 |
| GNAS | 0.0575 | 0.0676 | 0.0445 | 0.0851 | 0.0198 | 0.0059 | 0.1483 | 0.1598 |
| FAIM3 | 0.0001 | 0.0272 | 0.0018 | 0.2069 | 0.2054 | 0.3831 | 0.314 | −0.2369 |
| ETV1 | 0.0136 | 0.1691 | 0.0797 | −0.3344 | −0.3623 | −0.2357 | 0.0442 | 0.3183 |
| BCL6 | 0.0007 | 0.1446 | 0.027 | 0.447 | −0.359 | −0.4883 | 0.2586 | 0.1287 |
| SLC38A1 | 0.0133 | 0.0182 | 0.0054 | −0.4746 | −0.0455 | −0.3086 | 0.478 | 0.2367 |
| PDE7B | 0.0001 | 0.0016 | 0.0008 | 0.2561 | −0.0287 | −0.3054 | 0.2038 | −0.1832 |
| STAT1 | 0.0152 | 0.0195 | 0.0205 | 0.3762 | −0.0198 | −0.3058 | 0.1752 | −0.1676 |
| EIF3H | 0.0613 | 0.2247 | 0.1655 | −0.1658 | −0.3374 | −0.2692 | 0.4415 | 0.4914 |
| EID1 | 0.0051 | 0.0651 | 0.0099 | −0.0272 | −0.0565 | −0.0241 | 0.0609 | 0.1764 |
| ID3 | 0.0001 | 0.0004 | 0.0001 | −0.0134 | −0.0051 | −0.1564 | 0.377 | 0.1771 |
| PSAP | 0.0249 | 0.0539 | 0.016 | −0.0811 | −0.0025 | −0.2053 | 0.1912 | 0.2052 |
| DPP7 | −0.3978 | −0.3109 | −0.1431 | −0.0037 | −0.0006 | −0.056 | −0.3368 | 0.343 |
| PJA2 | 0.031 | 0.0421 | 0.0227 | −0.3023 | −0.268 | −0.0611 | 0.0781 | 0.0005 |
| TARDBP | 0.0509 | 0.0174 | 0.0984 | −0.0247 | −0.003 | −0.0624 | −0.0368 | 0.2196 |
| SRSF1 | 0.1179 | −0.4137 | 0.0735 | −0.1695 | −0.3563 | −0.3493 | 0.4526 | 0.421 |
| GABPB1 | 0.0148 | 0.0381 | 0.0058 | −0.0479 | −0.4275 | −0.1234 | −0.3436 | −0.1035 |
| RGS4 | 0.0001 | 0.0001 | 0.0001 | −0.3952 | −0.0058 | −0.0021 | −0.2055 | −0.0338 |
| SPTAN1 | 0.0351 | 0.2255 | 0.153 | −0.1103 | −0.0111 | −0.0415 | −0.0236 | −0.0076 |
| NFATC1 | 0.0001 | 0.0005 | 0.0001 | −0.012 | −0.0009 | −0.0047 | 0.3818 | −0.3159 |
| HAVCR2 | 0.048 | 0.1094 | 0.0349 | −0.0161 | −0.0006 | −0.0005 | −0.0349 | −1.00E−04 |
| PDCD1 | 0.0001 | 0.0328 | 0.0972 | −0.0712 | −1.00E−04 | −0.0077 | −0.0009 | −1.00E−04 |
| SRSF4 | 0.0006 | 0.0255 | 0.0074 | −0.3198 | −0.0888 | −0.1828 | −0.1201 | −0.1014 |
| GFOD1 | 0.0145 | 0.193 | 0.0222 | 0.2562 | −0.4242 | −0.1028 | −0.0427 | −0.1407 |
| MRPS21 | 0.0201 | 0.1464 | 0.0761 | −0.0619 | 0.4463 | −0.1624 | −0.0539 | −0.0006 |
| AP3S1 | 0.0061 | 0.0004 | 0.0025 | 0.4354 | −0.3337 | 0.211 | −0.0427 | −0.0016 |
| GPBP1 | 0.0038 | 0.0312 | 0.0151 | 0.3947 | 0.3732 | 0.2847 | −0.0203 | −1.00E−04 |
| BTLA | 0.1412 | 0.4237 | 0.1543 | −0.242 | −0.167 | 0.2844 | −0.0003 | −1.00E−04 |
| PAM | 0.0001 | 0.0219 | 0.0069 | 0.1068 | 0.1068 | −0.4177 | −0.2393 | −0.0006 |
| CBLB | 0.1169 | −0.183 | 0.3369 | 0.1511 | −0.1786 | 0.4112 | −0.2308 | −0.0004 |
| ATHL1 | 0.1179 | −0.4162 | 0.4466 | 0.0602 | 0.2744 | −0.0835 | −0.0021 | −0.001 |
| MGEA5 | 0.0487 | 0.0756 | 0.1481 | 0.0059 | 0.0918 | 0.1918 | −0.0954 | −0.0093 |
| IRF4 | 0.2331 | 0.2754 | 0.0717 | 0.2973 | 0.2343 | 0.3745 | −0.1268 | −1.00E−04 |
| UBE2F | 0.0107 | 0.0261 | 0.0039 | 0.2086 | 0.1827 | 0.1455 | −0.4132 | −0.1391 |
| SFXN1 | 0.0497 | 0.2332 | 0.1161 | 0.3959 | 0.4127 | −0.3629 | −0.0035 | −0.0662 |
| DGKH | 0.0019 | 0.0485 | 0.011 | 0.4355 | −0.2524 | 0.1137 | −0.0552 | −0.1051 |
| FCRL3 | 0.0001 | 0.0006 | 0.0001 | 0.159 | −0.0272 | −0.134 | −0.0424 | −0.0088 |
| PYHIN1 | 0.0001 | 0.0093 | 0.0023 | 0.0606 | 0.2723 | 0.3626 | −0.292 | −0.3693 |
| EIF1B | 0.0149 | 0.096 | 0.0915 | 0.1806 | −0.391 | −0.3403 | −0.0159 | −0.0367 |
| RAPGEF6 | 0.0075 | 0.202 | 0.0848 | 0.127 | −0.2386 | 0.2459 | −0.0356 | −0.056 |
| SNX9 | 0.099 | −0.3226 | 0.4407 | 0.0412 | −0.0912 | −0.3387 | −0.0305 | −0.0065 |
| IL6ST | 0.0243 | 0.2147 | 0.1732 | 0.0145 | −0.2006 | −0.4255 | 0.2999 | −0.035 |
| PTPN7 | 0.1817 | 0.2599 | 0.2809 | 0.4547 | −0.3064 | 0.3661 | −0.3863 | −0.0677 |
| CREM | 0.0045 | 0.0081 | 0.0047 | 0.0515 | 0.068 | 0.165 | −0.485 | −0.3915 |
| HNRPLL | 0.0166 | 0.0305 | 0.0068 | 0.2465 | 0.3424 | 0.4496 | 0.408 | −0.149 |
| FUT8 | 0.0156 | 0.0021 | 0.0081 | 0.1207 | 0.0804 | 0.1811 | −0.0548 | −0.0128 |
| LITAF | 0.0008 | 0.0005 | 0.0084 | 0.0431 | 0.1293 | 0.0194 | −0.2215 | −0.0039 |
| TSC22D1 | 0.0037 | 0.1164 | 0.0289 | 0.0001 | 0.2362 | 0.0448 | −0.1545 | −0.158 |
| TRAF5 | 0.0234 | −0.4801 | 0.154 | 0.1681 | 0.2183 | 0.2498 | 0.1356 | −0.0284 |
| ATP6V0B | 0.0331 | 0.0222 | 0.0748 | 0.2365 | 0.0276 | 0.2315 | −0.1334 | −0.0408 |
| SRSF6 | 0.0359 | 0.0443 | 0.0171 | 0.2861 | 0.4134 | −0.3609 | −0.0448 | −0.0608 |
| ELMO1 | 0.0001 | 0.0017 | 0.0085 | −0.2022 | 0.2642 | −0.3247 | −0.11 | −0.0878 |
| IRF8 | 0.0001 | 0.0001 | 0.0001 | −0.265 | −0.3089 | −0.2649 | −0.0505 | −0.001 |
| TAGAP | 0.0013 | 0.0065 | 0.0001 | 0.1585 | 0.3662 | −0.4354 | −0.0014 | −0.0004 |
| CADM1 | 0.0001 | 0.0043 | 0.0001 | 0.2899 | 0.1734 | 0.4366 | −0.2321 | −0.3866 |
| SPRY2 | 0.0001 | 0.0044 | 0.0163 | −0.4987 | 0.2094 | 0.1092 | 0.2825 | 0.4914 |
| CTLA4 | 0.0043 | 0.0271 | 0.0217 | −0.4126 | −0.4748 | −0.3087 | −0.4548 | −0.0972 |
| ANKRD10 | 0.0137 | 0.1915 | 0.1944 | −0.1961 | −0.0835 | −0.0327 | −0.4088 | −0.0742 |
| KLRK1 | 0.0313 | 0.4904 | 0.3958 | 0.22 | −0.0448 | −0.3704 | 0.3119 | −0.0463 |
| TP53INP1 | 0.0105 | 0.4566 | 0.1559 | 0.0947 | −0.1485 | −0.3713 | −0.4437 | −0.1516 |
| NR4A2 | 0.0281 | 0.0512 | 0.0126 | 0.1335 | −0.3848 | −0.3577 | −0.0167 | −0.0438 |
| ZNF292 | 0.0293 | 0.0561 | 0.0181 | 0.4131 | 0.2098 | 0.1227 | −0.0802 | −0.0184 |
| MIF4GD | 0.1244 | 0.0553 | 0.076 | 0.2113 | 0.0877 | 0.3779 | −0.0098 | −0.2079 |
| ING3 | 0.0352 | 0.0713 | 0.023 | 0.0209 | 0.0001 | 0.0001 | −0.1077 | −0.1147 |
| SQSTM1 | 0.1251 | 0.0684 | 0.014 | 0.011 | 0.0001 | 0.0001 | 0.4949 | 0.1462 |
| CLK4 | 0.0146 | 0.0267 | 0.0046 | 0.0026 | 0.0001 | 0.0016 | −0.0132 | −0.2005 |
| NCBP2 | 0.0761 | 0.0839 | 0.0864 | 0.3421 | 0.002 | 0.0733 | −0.3949 | −0.1621 |
| SET | 0.2578 | 0.3522 | 0.2514 | 0.3109 | 0.0002 | 0.0476 | −0.0041 | −0.2302 |
| PSME3 | 0.1703 | 0.1962 | 0.1704 | 0.0307 | 0.0041 | 0.0162 | −0.1147 | −0.4493 |
| IQCB1 | 0.4831 | 0.4272 | −0.4513 | 0.0004 | 0.0001 | 0.0002 | −0.0499 | 0.3017 |
| RGCC | 0.3816 | −0.2836 | −0.4288 | 0.0143 | 0.0001 | 0.0002 | 0.4972 | 0.397 |
| C20orf111 | −0.3416 | −0.0838 | −0.1112 | 0.0938 | 0.0005 | 0.0009 | −0.1324 | −0.1823 |
| MPP1 | −0.0342 | −0.0046 | −0.0178 | 0.0095 | 0.0024 | 0.004 | −0.0047 | −0.031 |
| CALR | −0.0938 | −0.2004 | −0.2372 | 0.0038 | 0.0001 | 0.0001 | −0.0004 | −0.0465 |
| TMEM160 | −0.3742 | −0.1052 | −0.1724 | 0.425 | 0.008 | 0.0818 | 0.3498 | 0.1857 |
| SRGN | 0.4184 | −0.0423 | −0.1241 | 0.1095 | 0.4445 | 0.4034 | −0.344 | −0.0757 |
| EWSR1 | 0.0118 | −0.4683 | 0.3428 | 0.0204 | 0.0434 | 0.0005 | 0.151 | −0.3127 |
| EZR | 0.2065 | 0.1578 | −0.4977 | 0.0828 | 0.1071 | 0.0429 | 0.398 | −0.1984 |
| FTSJ3 | 0.0579 | 0.1559 | 0.0424 | 0.0018 | 0.0001 | 0.0001 | −0.2009 | −0.3111 |
| LRMP | 0.0398 | −0.4503 | 0.3631 | 0.0799 | 0.0852 | 0.2464 | 0.3384 | −0.3955 |
| GBP2 | 0.0536 | 0.2695 | 0.2194 | 0.3615 | 0.2683 | −0.3024 | 0.3914 | −0.3319 |
| MPG | 0.0156 | 0.0846 | 0.1339 | 0.3225 | 0.0798 | 0.118 | 0.2381 | 0.1284 |
| RELA | 0.1242 | 0.1246 | 0.3455 | 0.1034 | 0.0027 | 0.0124 | 0.3078 | 0.2515 |
| KLHDC4 | −0.0566 | −0.4969 | 0.4231 | 0.0246 | 0.0001 | 0.0043 | −0.3618 | 0.3118 |
| PMS2P1 | 0.1954 | 0.4087 | 0.343 | 0.0001 | 0.0001 | 0.0003 | 0.044 | 0.0093 |
| CWF19L1 | −0.2621 | −0.3743 | −0.2442 | 0.0045 | 0.0001 | 0.0004 | −0.4888 | 0.3449 |
| AP2S1 | 0.376 | 0.4353 | 0.3762 | 0.0122 | 0.0017 | 0.0028 | 0.2261 | 0.1953 |
| RAE1 | 0.3297 | −0.4157 | −0.4357 | 0.0891 | 0.0028 | 0.1877 | −0.1612 | 0.1979 |
| TRIPI2 | 0.4872 | −0.4515 | 0.3477 | 0.1367 | 0.0001 | 0.0783 | 0.001 | 0.1979 |
| PDZD11 | 0.4459 | −0.428 | 0.4579 | 0.1482 | 0.0001 | 0.0471 | 0.0298 | −0.3748 |
| SPG21 | −0.0686 | −0.1491 | −0.0613 | 0.0328 | 0.0001 | 0.0067 | 0.031 | 0.0418 |
| RRM1 | −0.0625 | −0.0912 | −0.0124 | 0.3685 | 0.0052 | 0.2614 | 0.2791 | 0.0224 |
| SUB1 | −0.068 | −0.0596 | −0.0256 | 0.0942 | 0.0075 | 0.0401 | 0.33 | 0.1353 |
| RAB11FIP1 | −0.1635 | −0.078 | −0.1473 | 0.0408 | 0.0011 | 0.0107 | 0.0945 | 0.4335 |
| USO1 | −0.0583 | −0.0079 | −0.0229 | 0.0265 | 0.0116 | 0.0026 | 0.4763 | 0.3757 |
| NIPSNAP3A | −0.2286 | −0.0419 | −0.0675 | 0.0222 | 0.0001 | 0.0001 | −0.2446 | 0.35 |
| ANAPC13 | −0.2299 | −0.0255 | −0.0447 | 0.0533 | 0.0127 | 0.0319 | −0.0176 | −0.3776 |
| AEN | −0.193 | 0.3651 | −0.3762 | 0.0001 | 0.0001 | 0.0001 | 0.4418 | 0.1678 |
| SF3B4 | 0.1025 | 0.4691 | 0.108 | 0.009 | 0.0001 | 0.0059 | −0.4885 | 0.3146 |
| CAV1 | 0.0223 | 0.3773 | 0.0229 | 0.2151 | 0.0092 | 0.068 | −0.2259 | −0.2623 |
| PSPC1 | 0.0951 | 0.0492 | 0.0029 | 0.0062 | 0.0001 | 0.0001 | −0.2726 | −0.0611 |
| TFRC | 0.1156 | 0.1816 | 0.0998 | 0.0942 | 0.0044 | 0.0084 | −0.1361 | −0.2711 |
| WDR48 | 0.029 | 0.1374 | 0.0316 | 0.0005 | 0.0001 | 0.0001 | −1.00E−04 | −0.0182 |
| INO80C | 0.0911 | 0.0511 | 0.3572 | 0.0168 | 0.0001 | 0.0017 | −0.1439 | −0.0243 |
| NOP58 | 0.0108 | 0.0223 | 0.0161 | 0.0001 | 0.0019 | 0.0174 | −1.00E−04 | −1.00E−04 |
| NFAT5 | −0.4677 | 0.4798 | 0.4803 | 0.0037 | 0.0251 | 0.0826 | −0.0003 | −1.00E−04 |
| LBH | 0.3928 | −0.2824 | −0.4914 | 0.0917 | 0.0976 | 0.2768 | −0.0037 | −1.00E−04 |
| LMAM2 | −0.1971 | −0.3431 | −0.029 | 0.0449 | 0.0443 | 0.0885 | −0.0009 | −0.0008 |
| ACOT9 | −0.1807 | −0.2684 | −0.1787 | 0.086 | 0.0122 | 0.0852 | −0.0712 | −0.0041 |
| BRAP | 0.2021 | 0.4889 | 0.2018 | 0.0063 | 0.0001 | 0.0001 | −0.0151 | −0.0016 |
| SLC7A5 | 0.4611 | 0.4325 | 0.129 | −0.1765 | 0.0215 | 0.0985 | −0.0059 | −0.1303 |
| CCT5 | −0.177 | −0.4139 | −0.4199 | 0.2219 | 0.0024 | 0.097 | −0.02 | −0.2105 |
| NAT10 | 0.2976 | 0.4304 | 0.0875 | 0.1298 | 0.0004 | 0.0076 | −0.1838 | −0.1336 |
| YBX1 | −0.3785 | −0.3285 | −0.2602 | 0.3978 | 0.0001 | 0.0006 | −0.2889 | −0.3228 |
| IMPDH2 | 0.3501 | −0.2483 | −0.2791 | 0.0296 | 0.0001 | 0.0001 | −0.041 | −0.2288 |
| PPM1B | 0.4273 | −0.4218 | −0.2336 | 0.0219 | 0.0001 | 0.0002 | −0.0018 | −0.0075 |
| BANF1 | −0.2457 | 0.4633 | −0.191 | 0.1799 | 0.0023 | 0.1855 | −0.1311 | −0.4313 |
| PLEKHO2 | −0.0497 | −0.1204 | −0.0548 | 0.0362 | 0.0029 | 0.0039 | −0.0687 | −0.0962 |
| HSPBP1 | −0.1052 | −0.3355 | −0.2506 | 0.0287 | 0.0039 | 0.0048 | −0.0081 | −0.11 |
| JTB | −0.2771 | −0.4443 | 0.3985 | 0.2695 | 0.0245 | 0.1427 | −0.0099 | −0.0091 |
| SRA1 | −0.4976 | 0.4438 | 0.4086 | 0.0789 | 0.0025 | 0.0056 | −0.0018 | −0.0649 |
| METTL9 | −0.2828 | 0.4591 | −0.421 | 0.2095 | 0.0176 | 0.103 | −0.0064 | −0.0146 |
| SLC44A2 | −0.1145 | −0.1074 | −0.165 | 0.0779 | 0.1172 | 0.0381 | −0.0012 | −1.00E−04 |
| MYCBP | 0.4584 | 0.4097 | −0.4557 | 0.1697 | 0.0084 | 0.0502 | −0.0027 | −0.0003 |
| KIAA0101 | −0.4382 | 0.3884 | 0.4664 | −0.4808 | 0.0001 | 0.0673 | −0.013 | 0.116 |
| | | P-values from comparison of each tumor to all other tumors (sign indicates | | | | | | |
| | | direction of change) | | | | | | |
| | | mel89 | | | | | | |
| | | p-value | mel74 p-value | | | mel58 p-value | | |
| | | tumor/ | | | tumor/ | | | tumor/ |
| | Gene | circulation | Mel75 | viral | circulation | Mel75 | viral | circulation |
| | Names | (Baitch) | program | (Wherry) | (Baitch) | program | (Wherry) | (Baitch) |
| | Consistent | | | | | | | |
| | across | | | | | | | |
| | tumors | | | | | | | |
| | (FIG. 5E) | | | | | | | |
| | CXCL13 | 0.3983 | 0.0307 | 0.1966 | 0.0202 | 0.4271 | −0.2962 | −0.3637 |
| | TNFRSF1B | −0.4765 | −0.3705 | −0.0007 | −0.0045 | 0.2445 | 0.1596 | 0.4538 |
| | RGS2 | −0.1813 | 0.0349 | −0.234 | 0.2331 | 0.4468 | −0.2887 | −0.0962 |
| | TIGIT | −0.024 | 0.0619 | −0.4568 | −0.1443 | −0.1619 | −0.0013 | −0.0002 |
| | CD27 | −1.00E−04 | 0.0287 | 0.4399 | 0.174 | 0.0025 | 0.1082 | 0.0342 |
| | TNFRSF9 | −1.00E−04 | 0.0034 | −0.1815 | 0.046 | 0.0998 | 0.4337 | −0.3316 |
| | SLA | −0.1291 | 0.0559 | 0.2291 | 0.0912 | −1.00E−04 | −1.00E−04 | −1.00E−04 |
| | RNF19A | −0.0003 | 0.1032 | 0.3497 | 0.2096 | 0.3686 | 0.4048 | −0.1726 |
| | INPP5F | −0.0912 | 0.0154 | 0.0793 | 0.0068 | −0.0221 | −0.0213 | −0.0728 |
| | XCL2 | −0.2984 | 0.1977 | −0.05 | −0.2756 | −0.2637 | 0.1856 | 0.0264 |
| | HLA- | −0.0016 | 0.0013 | 0.3343 | 0.103 | 0.0606 | −0.2195 | 0.1986 |
| | DMA | | | | | | | |
| | FAM3C | −0.0235 | 0.277 | 0.3408 | −0.4743 | 0.2708 | −0.353 | −0.0607 |
| | UQCRC1 | −0.3551 | 0.1072 | 0.1175 | 0.1441 | 0.1631 | 0.3254 | 0.1005 |
| | WARS | −1.00E−04 | −0.0641 | −0.2312 | −0.1171 | 0.0053 | 0.1388 | 0.0413 |
| | EIF3L | 0.2346 | 0.1717 | 0.2765 | 0.388 | −0.1885 | −0.2698 | −0.4857 |
| | KCNK5 | 0.3032 | 0.0026 | 0.0045 | 0.0009 | −0.1044 | 0.3949 | −0.3235 |
| | TMB1M6 | −0.113 | 0.2006 | −0.1039 | 0.2303 | −0.0384 | −0.4732 | −0.0294 |
| | CD200 | −1.00E−04 | −0.3961 | −0.2096 | −0.3336 | 0.0037 | 0.0394 | −0.2597 |
| | ZC3H7A | −0.1488 | −0.3309 | 0.3825 | 0.3306 | 0.4053 | 0.0852 | 0.3174 |
| | SH2D1A | 0.0903 | 0.0122 | 0.0273 | 0.0949 | −0.0011 | −0.0049 | −0.048 |
| | ATP1B3 | −0.0165 | 0.0001 | 0.0001 | 0.0001 | 0.4712 | −0.0264 | −0.0037 |
| | MYO7A | 0.0258 | 0.0271 | −0.3183 | 0.0504 | 0.0134 | 0.0161 | 0.0004 |
| | THADA | −0.3724 | 0.0018 | 0.032 | 0.0033 | −1.00E−04 | −1.00E−04 | −1.00E−04 |
| | PARK7 | −0.2995 | −0.1392 | −0.2244 | −0.0594 | −0.3595 | −0.0592 | −0.0554 |
| | EGR2 | −0.0005 | 0.0596 | 0.101 | 0.0191 | 0.0739 | 0.0563 | 0.0739 |
| | FDFT1 | −0.0041 | 0.0071 | 0.0028 | 0.0116 | −0.0309 | −0.0387 | −0.0591 |
| | CRTAM | −0.1555 | 0.0001 | 0.268 | 0.0099 | −1.00E−04 | −1.00E−04 | −1.00E−04 |
| | IFI16 | 0.0085 | −0.0009 | −1.00E−04 | −1.00E−04 | 0.0407 | 0.0024 | 0.0033 |
| | variable | | | | | | | |
| | across | | | | | | | |
| | tumors | | | | | | | |
| | (FIG. 5F) | | | | | | | |
| | GMNN | 0.004 | 0.1531 | 0.063 | 0.2336 | 0.1884 | 0.442 | 0.1884 |
| | AFG3L1P | 0.0043 | −0.4821 | −0.1021 | 0.0407 | −0.3377 | −0.0646 | 0.1039 |
| | CSRP1 | 0.0012 | −0.1946 | 0.3689 | −0.1347 | −0.0915 | −0.118 | −0.3935 |
| | RBM5 | 0.0166 | −0.2646 | −0.0234 | −0.0421 | −0.2612 | −0.2396 | 0.1098 |
| | AP1M1 | 0.0004 | −0.2514 | −0.0173 | −0.2783 | 0.4193 | −0.4828 | 0.4638 |
| | NUCB2 | 0.0314 | 0.4314 | −0.3709 | 0.4119 | 0.2533 | 0.0337 | 0.0928 |
| | NOP10 | 0.0029 | −0.3846 | −0.0607 | −0.1355 | 0.0082 | 0.0038 | 0.1462 |
| | GFM1 | 0.1448 | −0.0803 | −0.0331 | −0.0629 | 0.0241 | 0.3868 | 0.2105 |
| | DHRS7 | 0.0084 | −0.1701 | −0.0193 | −0.1031 | 0.2959 | 0.2512 | 0.1869 |
| | SSU72 | 0.0003 | −0.008 | −0.0003 | −0.003 | 0.4769 | 0.1685 | −0.3324 |
| | SBDS | 0.0004 | −0.0435 | −0.006 | −0.0172 | −0.2245 | 0.1463 | −0.331 |
| | ATP6V1B2 | 0.0086 | −0.0029 | −0.1052 | −0.1432 | −0.2362 | −0.2485 | −0.2362 |
| | VAPA | 0.0269 | −0.0438 | −0.0065 | −0.0002 | −0.0506 | −0.2917 | −0.0067 |
| | CSNK2A1 | 0.2428 | 0.124 | −0.0882 | −0.3671 | −0.4186 | −0.3409 | −0.276 |
| | LINC00339 | 0.0383 | −0.1203 | −0.0513 | 0.3244 | −0.0641 | −0.0511 | −0.0571 |
| | MRPL4 | 0.0899 | 0.3839 | −0.3913 | 0.0152 | 0.2456 | −0.0029 | −0.0795 |
| | PPP1R2 | 0.0231 | −0.4671 | −0.3808 | −0.1225 | −0.3305 | −0.136 | −0.1073 |
| | SMG1 | 0.0024 | 0.2378 | 0.3834 | −0.1629 | −0.0528 | −0.1564 | −0.1636 |
| | OIP5- | 0.0294 | −0.1076 | −0.0825 | 0.2108 | −0.0033 | −0.0019 | −0.0041 |
| | AS1 | | | | | | | |
| | LPAR2 | 0.0002 | 0.3122 | 0.0999 | 0.4376 | −1.00E−04 | −1.00E−04 | −0.0335 |
| | LSMD1 | 0.0254 | −0.2627 | −0.0076 | −0.0675 | −1.00E−04 | −1.00E−04 | −0.0007 |
| | STAG3L4 | 0.0004 | −0.3089 | −0.1532 | −0.3581 | −0.0104 | −0.0035 | −0.0062 |
| | P4HB | 0.0005 | −0.4788 | 0.3977 | 0.1387 | −0.0167 | −0.021 | −0.003 |
| | SKP1 | 0.0154 | −0.3198 | −0.0151 | 0.3509 | −0.0816 | −0.3082 | −1.00E−04 |
| | PTBP1 | 0.0072 | −0.3252 | 0.1796 | 0.4811 | −0.021 | −0.0075 | −0.0047 |
| | TSTA3 | 0.0001 | 0.0012 | 0.0002 | 0.0237 | −0.032 | −0.0206 | −0.0261 |
| | TBCB | 0.0007 | 0.005 | 0.0003 | 0.0001 | −0.1895 | −0.2302 | −1.00E−04 |
| | SMC5 | 0.0605 | 0.1683 | 0.0698 | 0.0739 | −0.0456 | −0.1039 | −0.4043 |
| | KLHDC2 | 0.0368 | 0.3407 | 0.0447 | 0.2635 | −0.0655 | −0.1308 | −0.3169 |
| | MPV17 | 0.0005 | 0.0005 | 0.02 | 0.1601 | −0.0922 | −0.0617 | −0.3365 |
| | RBPJ | 0.0075 | 0.1409 | 0.372 | 0.1009 | −0.094 | −0.0413 | −0.0419 |
| | POP5 | 0.05 | 0.0669 | 0.4084 | 0.1823 | 0.4812 | −0.2965 | −0.0784 |
| | PPAPDC1B | 0.0261 | 0.048 | 0.1217 | 0.3555 | −1.00E−04 | −0.009 | −0.0044 |
| | IMP3 | 0.1001 | 0.1277 | 0.0179 | 0.0597 | −0.0029 | −0.0017 | −0.001 |
| | RNPS1 | −0.4743 | 0.2279 | 0.0005 | 0.0587 | −1.00E−04 | −1.00E−04 | −1.00E−04 |
| | NFE2L2 | 0.4335 | 0.2273 | 0.0017 | 0.07 | −1.00E−04 | −1.00E−04 | −1.00E−04 |
| | SOD1 | −0.1439 | 0.3402 | 0.3845 | −0.4253 | −1.00E−04 | −1.00E−04 | −1.00E−04 |
| | CD8B | 0.0772 | −0.4645 | 0.1771 | −0.1336 | −1.00E−04 | −1.00E−04 | −0.0186 |
| | PTPN6 | 0.1356 | 0.021 | −0.2108 | 0.4185 | −0.03 | −0.014 | −0.3063 |
| | HSPA1B | 0.4475 | 0.0001 | 0.055 | 0.0003 | −0.0003 | −0.0151 | −0.014 |
| | CD2BP2 | −0.196 | 0.1398 | 0.4007 | 0.1204 | −0.0621 | −0.0167 | −0.0631 |
| | ALDOA | −0.3407 | 0.2764 | 0.3881 | −0.3662 | −0.2093 | −0.0042 | −0.2345 |
| | ZFP36L1 | 0.2357 | −0.4689 | −0.2409 | 0.3495 | −0.38 | −0.1202 | −0.0177 |
| | HSPB1 | 0.3519 | 0.0003 | 0.0069 | 0.0009 | −0.1063 | −0.0932 | −0.0017 |
| | HSPA6 | 0.4776 | 0.0104 | 0.2195 | 0.0105 | −0.2872 | −0.2872 | −0.2872 |
| | ARHGEF1 | −0.3478 | 0.2938 | −0.4655 | 0.1659 | 0.3643 | −0.3728 | −0.2799 |
| | LUC7L3 | 0.2498 | 0.0667 | 0.1515 | 0.2121 | −0.3077 | −0.3836 | −0.0648 |
| | GPR174 | 0.2228 | 0.3676 | −0.2747 | 0.1659 | −0.0158 | −0.0071 | −0.0017 |
| | ENTPD1 | 0.2983 | 0.0001 | 0.0017 | 0.0027 | −0.0009 | −0.0189 | −0.0232 |
| | RASSF5 | −0.1779 | 0.2249 | −0.4755 | 0.1092 | −0.0399 | −0.0048 | −0.1204 |
| | IPCEF1 | −0.0973 | −0.4382 | 0.1925 | 0.0657 | −0.2933 | −0.1473 | −0.0687 |
| | ARNT | −0.0598 | 0.2893 | 0.3013 | 0.1755 | 0.4915 | −0.0284 | −0.0249 |
| | NAB1 | −0.2593 | 0.03 | 0.2629 | 0.0456 | −0.1623 | −0.0304 | −0.081 |
| | APLP2 | −0.1145 | 0.002 | 0.0577 | 0.0097 | −0.2554 | −0.0256 | −0.2598 |
| | PRKCH | −0.0004 | 0.0266 | 0.0793 | 0.1044 | −0.0719 | −0.0637 | −0.0114 |
| | SEMA4A | −0.0098 | 0.0001 | 0.0029 | 0.003 | 0.4652 | −0.074 | −0.1757 |
| | PPP1CC | −0.0035 | 0.0018 | 0.0006 | 0.0001 | −0.0647 | −0.0093 | −0.0064 |
| | LAG3 | −0.0481 | 0.0031 | 0.0062 | 0.0026 | −0.4258 | −0.0077 | −0.049 |
| | HSPA1A | −0.0108 | 0.0001 | 0.0001 | 0.0001 | −0.0466 | −0.024 | −0.065 |
| | SNAP47 | −0.0015 | 0.001 | 0.0357 | 0.1147 | −0.0216 | −0.0156 | −0.035 |
| | CCL4L2 | −0.0014 | 0.0087 | 0.0119 | 0.0377 | −0.0037 | −0.0029 | −1.00E−04 |
| | ARID4B | −0.0566 | 0.0582 | 0.1886 | 0.0472 | −0.0534 | −0.0932 | −0.0046 |
| | LYST | −0.0014 | 0.001 | 0.2387 | 0.1652 | −0.0009 | −1.00E−04 | −1.00E−04 |
| | NMB | −0.2737 | 0.0088 | 0.0425 | 0.0976 | −0.0274 | −0.0274 | −0.0274 |
| | LIMS1 | −0.4231 | 0.1279 | 0.0166 | 0.0995 | −0.4037 | −0.0847 | −0.0395 |
| | ITK | −0.4003 | 0.0006 | 0.0001 | 0.0001 | −0.0231 | −1.00E−04 | −0.0005 |
| | RILPL2 | −0.1891 | 0.0137 | 0.0193 | 0.0368 | −0.2425 | −0.0171 | −0.3285 |
| | RGS3 | −0.215 | 0.4035 | 0.0496 | 0.3993 | 0.372 | −0.0028 | −0.3945 |
| | TRAT1 | −0.0025 | 0.0413 | 0.1148 | 0.1308 | −0.2189 | −0.1665 | 0.4073 |
| | ELF1 | −0.0039 | 0.0791 | 0.3842 | 0.0802 | −0.0125 | −0.2311 | −0.4031 |
| | OSBPL3 | −0.0076 | 0.1909 | −0.2977 | 0.4306 | −0.317 | −0.2557 | −0.3181 |
| | BIRC3 | −0.0513 | 0.0605 | −0.3405 | 0.2832 | −0.401 | −0.2932 | −0.123 |
| | PTGER4 | −0.0053 | 0.3421 | 0.1446 | 0.2333 | −0.1815 | −0.0895 | −0.1027 |
| | SERINC3 | −1.00E−04 | 0.001 | 0.0012 | 0.0518 | −0.0022 | −0.073 | −0.1248 |
| | MED7 | −0.0003 | −0.3679 | 0.2069 | 0.0277 | 0.4739 | −0.4247 | −0.1413 |
| | DDX3X | −0.0027 | 0.1202 | 0.0012 | 0.0001 | 0.4489 | −0.0628 | −0.0403 |
| | THEM6 | −1.00E−04 | 0.0296 | 0.0119 | 0.0004 | −0.1814 | −0.1933 | −0.0447 |
| | P4HA1 | −0.0038 | 0.2371 | 0.0041 | 0.0003 | 0.2664 | 0.4252 | 0.2664 |
| | HIBCH | −0.0002 | 0.008 | 0.0053 | 0.0001 | 0.0608 | 0.1542 | 0.303 |
| | VCAM1 | −0.0081 | 0.0002 | 0.0657 | 0.004 | 0.3427 | −0.4746 | −0.4275 |
| | FABP5 | −0.0882 | 0.0001 | 0.2995 | 0.2092 | 0.1878 | −0.0656 | 0.4217 |
| | NOL7 | −0.0556 | 0.0001 | 0.0001 | 0.0001 | −0.2186 | −0.0018 | −0.0403 |
| | SEC14L1 | −0.1781 | 0.005 | 0.0019 | 0.0011 | −0.1134 | −0.1188 | −0.3572 |
| | UBA2 | 0.276 | 0.0078 | 0.0002 | 0.0002 | −0.1417 | −0.0762 | −0.1107 |
| | CDCA4 | 0.0611 | 0.0018 | 0.0501 | 0.0501 | −0.0695 | 0.3345 | 0.4639 |
| | ATP5I | −0.3296 | 0.1231 | 0.0739 | 0.0005 | 0.4643 | −0.0835 | −0.0035 |
| | ALKBH3 | −0.1489 | 0.0354 | 0.0024 | 0.0036 | 0.4315 | 0.3951 | −0.132 |
| | DND1 | −0.3032 | 0.0038 | 0.0007 | 0.0007 | −0.4077 | −0.1166 | −0.2939 |
| | RNF185 | −0.1602 | 0.0223 | 0.0008 | 0.0004 | −0.1286 | −0.1611 | 0.3973 |
| | AFAP1L2 | −0.3306 | 0.0001 | 0.0001 | 0.0001 | −0.3901 | −0.2275 | −0.139 |
| | GLOD4 | 0.4954 | 0.0001 | 0.0017 | 0.0001 | −0.0476 | −0.0308 | −0.1533 |
| | PIP5K1A | −0.3296 | 0.0002 | 0.0001 | 0.0001 | −0.0283 | −0.126 | 0.3006 |
| | ATF4 | 0.1382 | 0.0001 | 0.0001 | 0.0001 | −0.2516 | −0.0401 | −0.441 |
| | PIGO | −0.3289 | 0.0001 | 0.0001 | 0.0001 | −0.3448 | −0.0918 | −0.3448 |
| | OPA1 | −0.2373 | 0.0026 | 0.0029 | 0.0019 | −0.2068 | −0.2052 | −0.3232 |
| | CCT3 | −0.0552 | 0.0001 | 0.0001 | 0.0001 | −0.0222 | −0.0119 | −0.104 |
| | EXOSC6 | −0.47 | 0.0001 | 0.0001 | 0.0001 | −0.0022 | −0.0471 | −0.0885 |
| | KIAA1429 | −0.1917 | 0.0143 | 0.0001 | 0.0001 | −0.0753 | 0.1958 | −0.112 |
| | NDFIP2 | −0.1181 | 0.0915 | 0.1478 | 0.0257 | −0.228 | −0.2705 | −0.006 |
| | TMEM222 | −0.3053 | 0.0093 | 0.0884 | 0.0001 | −0.2535 | −0.0167 | −0.0217 |
| | MYO1G | −0.3489 | 0.0001 | 0.0001 | 0.0001 | −1.00E−04 | −1.00E−04 | −1.00E−04 |
| | LBR | 0.4131 | 0.0733 | 0.031 | 0.0009 | −0.0076 | −0.0076 | −0.0049 |
| | EXT2 | 0.0722 | 0.0313 | 0.0144 | 0.0144 | −0.1512 | −0.014 | −0.0042 |
| | SARDH | −0.3145 | 0.1776 | 0.3563 | 0.0199 | 0.3768 | −0.1201 | −0.0215 |
| | POLR2I | 0.4334 | 0.0017 | 0.0001 | 0.0001 | −0.002 | −0.0133 | −0.0285 |
| | HNRNPD | 0.2702 | 0.0778 | 0.0001 | 0.0001 | −0.3149 | −0.0736 | −0.0634 |
| | NAAA | −0.0531 | 0.0028 | 0.001 | 0.0001 | −0.0504 | −0.0459 | −0.1079 |
| | ARID5A | −0.3703 | 0.0002 | 0.0001 | 0.0001 | 0.403 | −0.371 | 0.2668 |
| | PDRG1 | −0.3741 | 0.0004 | 0.0001 | 0.0001 | 0.2061 | 0.1895 | 0.2061 |
| | BCAP31 | 0.138 | 0.0117 | 0.0332 | 0.0042 | 0.1817 | 0.1686 | −0.3479 |
| | UQCRFS1 | 0.3234 | 0.0042 | 0.0102 | 0.0006 | 0.1627 | 0.2109 | 0.1627 |
| | SNRNP40 | 0.3361 | 0.0261 | 0.0608 | 0.0001 | 0.0059 | 0.0472 | 0.041 |
| | ASB8 | −0.112 | 0.3666 | 0.0806 | 0.0583 | 0.0404 | 0.0215 | 0.0075 |
| | MRPL52 | −0.0854 | 0.0119 | 0.0002 | 0.0001 | 0.0076 | 0.026 | 0.0034 |
| | TUG1 | −0.4266 | 0.0672 | 0.0103 | 0.0001 | 0.292 | 0.1542 | 0.1173 |
| | CCND2 | −0.045 | 0.0395 | 0.0142 | 0.0005 | 0.3164 | 0.0374 | −0.42 |
| | NAA20 | −0.0283 | 0.0015 | 0.0001 | 0.0001 | 0.0219 | 0.2703 | 0.3452 |
| | HLA- | −0.3248 | 0.0001 | 0.0002 | 0.0001 | 0.0015 | 0.0746 | 0.013 |
| | DPA1 | | | | | | | |
| | TOX | −0.0017 | 0.0052 | 0.0163 | 0.0001 | 0.3272 | 0.4052 | 0.0035 |
| | TMEM205 | −0.1677 | 0.0168 | 0.0012 | 0.0001 | 0.1239 | 0.0135 | 0.1239 |
| | TPI1 | −0.0807 | 0.2587 | −0.1576 | 0.4564 | 0.0244 | 0.0644 | 0.3177 |
| | HADHA | −0.3573 | 0.1645 | 0.1619 | 0.0974 | 0.0022 | 0.21 | 0.0012 |
| | STAT3 | 0.2292 | 0.1084 | 0.0253 | 0.4449 | 0.0906 | 0.0735 | 0.0057 |
| | GMDS | −0.0026 | −0.2109 | −0.0656 | −0.1852 | 0.0001 | 0.0001 | 0.0001 |
| | SIRPG | −0.2851 | −0.1594 | −0.0976 | −0.278 | 0.0045 | 0.0015 | 0.0001 |
| | ITM2A | −0.1526 | −0.0698 | −0.0012 | −0.069 | 0.157 | 0.2656 | 0.0001 |
| | TBC1D4 | −0.3658 | −0.1544 | −0.0065 | −0.0079 | 0.0033 | 0.0008 | 0.0433 |
| | HNRNPM | −0.0849 | −0.0366 | −0.0172 | −0.0682 | 0.2081 | 0.0927 | 0.4216 |
| | ASB2 | −0.3882 | −0.3974 | −0.4001 | −0.4602 | 0.1802 | −0.4817 | 0.2272 |
| | IGFLR1 | −0.0049 | −0.1111 | −1.00E−04 | −0.0012 | 0.0098 | 0.0047 | 0.0014 |
| | CD2 | −0.0439 | −0.2105 | −0.0276 | −0.0391 | 0.0019 | 0.0001 | 0.0342 |
| | COTL1 | −0.071 | −0.0953 | −0.033 | −0.0289 | 0.0001 | 0.0001 | 0.0001 |
| | PBRM1 | −0.2478 | −0.0487 | −0.0546 | −0.0048 | 0.0039 | 0.0009 | 0.0632 |
| | DOT | 0.3591 | −0.1652 | −0.0005 | −0.009 | 0.0382 | 0.2155 | 0.0016 |
| | LMF2 | 0.4718 | −0.0311 | −0.0018 | −0.0167 | 0.1788 | 0.3654 | 0.001 |
| | TAF15 | −0.4413 | −0.0374 | −0.0008 | −0.0069 | 0.0985 | 0.2079 | 0.1703 |
| | H2AFY | 0.0074 | −0.0004 | −1.00E−04 | −1.00E−04 | 0.3297 | 0.3344 | 0.2405 |
| | CEP57 | 0.4316 | −0.0012 | −1.00E−04 | −0.0014 | 0.2788 | 0.2247 | 0.2091 |
| | AMDHD2 | 0.0038 | −0.022 | −0.0027 | −0.0217 | 0.0354 | 0.0001 | 0.2651 |
| | SERINC1 | 0.0148 | −0.352 | −0.0334 | −0.1446 | 0.1357 | 0.1727 | 0.0454 |
| | CKS2 | 0.353 | −0.1864 | −0.1208 | −0.0861 | 0.4921 | −0.1094 | 0.2659 |
| | PTPN11 | 0.1915 | −0.3803 | −0.0597 | −0.0246 | 0.0829 | −0.3613 | 0.244 |
| | DDX3Y | 0.2473 | −0.0317 | −0.0352 | −0.0205 | 0.2156 | 0.0702 | −0.4669 |
| | IRF9 | 0.2927 | −0.0046 | −0.0004 | −1.00E−04 | −0.0996 | −0.1003 | −0.4866 |
| | FYN | 0.0446 | −0.089 | −0.0017 | −1.00E−04 | −0.0005 | −0.0007 | −0.0369 |
| | HSPD1 | −0.4631 | −0.0675 | −1.00E−04 | −0.0076 | −0.265 | −0.0188 | −0.1024 |
| | FPGS | −0.4765 | 0.3785 | 0.4239 | 0.3387 | −0.2479 | −0.161 | 0.2987 |
| | CCT2 | 0.2599 | 0.1384 | −0.3221 | −0.0815 | −0.0315 | −0.1647 | −0.1171 |
| | GNAS | 0.1477 | 0.0369 | −0.1147 | −0.2145 | −0.1606 | −0.0008 | −0.0002 |
| | FAIM3 | −0.3308 | −0.0397 | −0.0028 | −0.0142 | −0.1334 | −0.0255 | −1.00E−04 |
| | ETV1 | 0.4475 | −0.4655 | −0.058 | −0.058 | −0.1993 | −0.0148 | −0.0011 |
| | BCL6 | 0.2076 | −0.2198 | −0.155 | −0.155 | −0.0343 | −0.025 | −0.0285 |
| | SLC38A1 | 0.2843 | −0.069 | −0.0203 | −0.0984 | −0.1013 | −0.0674 | −0.0004 |
| | PDE7B | 0.2414 | −0.0211 | −0.0302 | −0.0011 | −1.00E−04 | −0.0004 | −0.0062 |
| | STAT1 | −0.3559 | −0.0571 | −0.0226 | −0.0018 | −0.3875 | −1.00E−04 | −0.0848 |
| | EIF3H | −0.4331 | −0.0553 | −0.1158 | −0.2226 | 0.1259 | −0.0505 | −0.062 |
| | EID1 | 0.2539 | −0.4621 | −0.0089 | −0.3579 | −0.0551 | −0.0004 | −0.0028 |
| | ID3 | 0.081 | 0.2647 | −0.0014 | −0.0603 | −0.0009 | −0.0204 | −1.00E−04 |
| | PSAP | 0.1522 | −0.2252 | −0.467 | −0.1392 | −0.2235 | −0.04 | −0.0838 |
| | DPP7 | −0.2813 | −0.0192 | −0.0425 | −0.0887 | −0.1471 | −0.0229 | −0.3627 |
| | PJA2 | 0.0021 | 0.4328 | −0.1306 | −0.2828 | −0.1854 | −0.4278 | −0.0232 |
| | TARDBP | 0.4618 | −0.1874 | −0.1654 | −0.3867 | 0.0313 | −0.2905 | −0.0668 |
| | SRSF1 | 0.3271 | −0.0764 | 0.3724 | −0.2089 | 0.2995 | 0.4141 | −0.1469 |
| | GABPB1 | −0.0798 | −0.2629 | −0.4536 | 0.4159 | 0.1397 | 0.345 | −0.0324 |
| | RGS4 | −0.1158 | −0.2765 | −0.1393 | −0.408 | 0.1087 | 0.396 | −0.2693 |
| | SPTAN1 | −0.0028 | −0.3223 | −0.1084 | −0.1473 | −0.2418 | −0.075 | −0.0974 |
| | NFATC1 | −0.4556 | 0.2826 | 0.2825 | 0.3826 | −0.3091 | −0.1764 | 0.3065 |
| | HAVCR2 | −0.0028 | 0.0086 | 0.3346 | 0.0343 | −0.4481 | −0.061 | −0.474 |
| | PDCD1 | −1.00E−04 | 0.0053 | 0.325 | 0.2642 | 0.1303 | 0.4795 | −0.4433 |
| | SRSF4 | −0.0008 | 0.2112 | −0.3539 | 0.4595 | 0.0015 | 0.0002 | 0.0002 |
| | GFOD1 | −0.0642 | 0.3908 | 0.4082 | 0.3676 | 0.0551 | 0.0789 | 0.0082 |
| | MRPS21 | −0.0467 | 0.3083 | 0.3032 | 0.0986 | 0.2523 | 0.1299 | 0.0023 |
| | AP3S1 | −0.0367 | 0.14 | −0.2439 | 0.4976 | 0.0713 | 0.2382 | 0.0837 |
| | GPBP1 | −1.00E−04 | 0.343 | 0.3824 | 0.351 | −0.294 | −0.2655 | 0.2116 |
| | BTLA | −1.00E−04 | −0.4738 | −0.4148 | 0.3682 | 0.4305 | 0.359 | −0.2347 |
| | PAM | −0.001 | 0.0089 | 0.0211 | 0.02 | 0.3983 | 0.2329 | −0.1937 |
| | CBLB | −0.0101 | 0.0984 | −0.1037 | −0.3726 | 0.1585 | −0.494 | 0.4965 |
| | ATHL1 | −0.0007 | 0.4401 | −0.1026 | −0.1269 | 0.3386 | −0.1629 | 0.3843 |
| | MGEA5 | −0.0605 | −0.0481 | −0.0219 | −0.0002 | 0.2711 | 0.1697 | 0.3136 |
| | IRF4 | −0.0103 | −0.0066 | −0.013 | −0.0097 | 0.2486 | 0.4581 | 0.1895 |
| | UBE2F | 0.4106 | 0.2627 | −0.0542 | −0.1227 | 0.3626 | 0.4606 | 0.0802 |
| | SFXN1 | −0.0114 | −0.1413 | −0.0665 | −0.0415 | 0.3946 | 0.2155 | 0.105 |
| | DGKH | −0.0618 | −0.0585 | −0.1165 | −0.0353 | 0.3957 | −0.321 | −0.4282 |
| | FCRL3 | −0.0019 | −0.0358 | −0.0003 | −0.0003 | 0.4889 | −0.3527 | 0.0854 |
| | PYHIN1 | 0.3211 | 0.0354 | −0.3647 | 0.3622 | −0.1836 | 0.0837 | 0.3475 |
| | EIF1B | −0.0133 | 0.3358 | −0.0973 | −0.084 | 0.2619 | 0.2858 | 0.4961 |
| | RAPGEF6 | −0.0127 | −0.2199 | −0.3916 | −0.4647 | −0.4059 | 0.2278 | −0.3354 |
| | SNX9 | −0.1005 | −0.0339 | −0.0164 | −0.0637 | −0.3371 | −0.2421 | −0.1756 |
| | IL6ST | 0.3074 | −0.0178 | −0.0075 | −0.002 | −0.2547 | −0.1458 | −0.0424 |
| | PTPN7 | −0.0215 | −0.3073 | −1.00E−04 | −0.0133 | −0.4748 | −0.1305 | −0.0886 |
| | CREM | −0.4587 | −0.2094 | −0.0007 | −0.0018 | 0.1508 | −0.3835 | −0.2928 |
| | HNRPLL | −0.1422 | −0.0463 | −0.0013 | −1.00E−04 | −0.4825 | 0.3446 | 0.431 |
| | FUT8 | −0.0007 | −0.0557 | −1.00E−04 | −1.00E−04 | −0.1295 | −0.0632 | −0.3494 |
| | LITAF | −0.012 | −0.4642 | −0.077 | −0.3241 | −0.4046 | −0.071 | −1.00E−04 |
| | TSC22D1 | −0.1564 | 0.4824 | −0.4781 | −0.2828 | −0.0943 | −0.0943 | −0.0943 |
| | TRAF5 | −0.1426 | −0.2898 | −0.2942 | −0.2882 | −0.0202 | −0.1108 | −0.0755 |
| | ATP6V0B | −0.2396 | −0.2096 | 0.4486 | −0.4334 | −0.3823 | −0.0095 | −0.2905 |
| | SRSF6 | −0.0249 | 0.4553 | −0.2632 | −0.4161 | 0.3628 | −0.0214 | −0.2126 |
| | ELMO1 | −0.1856 | 0.2349 | −0.4556 | 0.3224 | −0.4806 | −0.2442 | −0.1741 |
| | IRF8 | −0.0626 | −0.1076 | −0.112 | −0.1849 | −0.2048 | −0.158 | −0.029 |
| | TAGAP | −0.0047 | −0.1065 | −0.0076 | −0.1496 | 0.3694 | −0.0341 | −0.0862 |
| | CADM1 | −0.0191 | −0.1972 | −0.2188 | −0.0725 | −0.106 | −0.1703 | −0.1647 |
| | SPRY2 | −0.3215 | 0.3129 | −0.1812 | 0.4752 | −0.0594 | −0.0112 | −0.0476 |
| | CTLA4 | −0.0796 | 0.3575 | −0.0425 | −0.2182 | −0.0083 | −0.0014 | −0.0149 |
| | ANKRD10 | −0.173 | 0.3787 | −0.0714 | −0.1285 | −0.0986 | −0.1351 | −0.115 |
| | KLRK1 | −0.1951 | −0.3913 | −0.1428 | −0.0717 | −0.0158 | −0.0106 | −1.00E−04 |
| | TP53INP1 | −0.2126 | 0.365 | −0.473 | 0.4267 | −0.0628 | −0.0005 | −0.0005 |
| | NR4A2 | −0.0821 | −0.0833 | −0.2007 | 0.422 | −0.0036 | −0.0004 | −1.00E−04 |
| | ZNF292 | −0.0394 | −0.2607 | −0.492 | −0.2302 | −1.00E−04 | −1.00E−04 | −1.00E−04 |
| | MIF4GD | 0.2737 | −0.1073 | −0.3075 | −0.1209 | −0.0081 | −1.00E−04 | −0.0008 |
| | ING3 | −0.426 | −0.419 | −0.0282 | −0.0479 | −0.2343 | −0.0012 | −0.002 |
| | SQSTM1 | 0.3767 | 0.1021 | −0.0392 | −0.3686 | −0.0094 | −0.0329 | −0.3804 |
| | CLK4 | −0.084 | −0.0857 | −0.0011 | −0.0157 | −0.06 | −0.0987 | −0.1181 |
| | NCBP2 | −0.4148 | −0.268 | −0.0255 | −0.2678 | 0.495 | −0.205 | −0.4967 |
| | SET | −0.3493 | −0.0822 | −0.0597 | −0.3679 | 0.1984 | 0.0099 | 0.2821 |
| | PSME3 | −0.225 | 0.3115 | −0.0552 | −0.1349 | 0.4803 | 0.1526 | 0.4803 |
| | IQCB1 | −0.1381 | −0.1305 | −0.0004 | −0.0274 | −0.0417 | 0.4761 | −0.2305 |
| | RGCC | 0.4057 | −0.1039 | −0.1815 | −0.1789 | −0.1373 | 0.1706 | −0.3813 |
| | C20orf111 | −0.0571 | −0.2769 | −0.0386 | −0.3185 | −0.3333 | −0.3333 | −0.3333 |
| | MPP1 | −0.0317 | −0.0996 | −0.0147 | −0.0252 | 0.1903 | 0.1521 | 0.1903 |
| | CALR | −0.0032 | −0.1132 | −0.2578 | −0.0533 | 0.0063 | 0.4329 | 0.0868 |
| | TMEM160 | −0.2813 | 0.3736 | −0.1531 | −0.0459 | 0.0039 | −0.1839 | 0.4527 |
| | SRGN | −0.0461 | 0.1475 | −0.0042 | −0.0217 | 0.0458 | 0.3993 | −0.0806 |
| | EWSR1 | −0.1578 | 0.4479 | −0.0423 | −0.0128 | 0.0094 | −0.4406 | 0.0233 |
| | EZR | −0.1058 | −0.0086 | −0.0006 | −1.00E−04 | −0.3525 | −0.418 | 0.2642 |
| | FTSJ3 | −0.4689 | −0.0079 | −0.0002 | −0.0034 | 0.3242 | 0.3076 | 0.3242 |
| | LRMP | −0.3517 | −0.1103 | −0.0002 | −0.0069 | 0.136 | 0.3452 | 0.1464 |
| | GBP2 | −0.304 | −0.0105 | −0.0053 | −0.0006 | 0.1358 | −0.2255 | 0.0972 |
| | MPG | −0.3192 | −0.1465 | −0.2823 | −0.0066 | 0.1816 | 0.018 | 0.2102 |
| | RELA | 0.1577 | −0.0034 | −0.003 | −0.0012 | −0.1369 | 0.0567 | −0.1801 |
| | KLHDC4 | 0.0837 | −1.00E−04 | −1.00E−04 | −0.0002 | −0.2458 | −0.2458 | −0.2458 |
| | PMS2P1 | 0.0786 | −0.0509 | −0.0676 | −0.017 | −0.2348 | −0.1039 | −0.3531 |
| | CWF19L1 | 0.4094 | −0.0457 | −0.3854 | −0.0199 | −0.0998 | −0.1908 | −0.0233 |
| | AP2S1 | −0.4831 | −0.2352 | −0.0259 | −0.2703 | −0.0023 | −0.0009 | −0.0022 |
| | RAE1 | 0.4225 | −0.285 | −0.1001 | −0.3787 | −0.0137 | −0.0033 | −0.1395 |
| | TRIPI2 | 0.1145 | −0.3894 | −0.4533 | −0.4119 | −0.01 | −1.00E−04 | −0.1613 |
| | PDZD11 | 0.1037 | 0.1485 | −0.3744 | −0.4908 | −0.1807 | −0.0526 | −0.2636 |
| | SPG21 | 0.0556 | 0.0919 | −0.3145 | −0.4868 | −0.3226 | −0.4651 | −0.1175 |
| | RRM1 | −0.3932 | −0.4764 | −0.4102 | 0.3325 | −0.1783 | −0.1488 | −0.2141 |
| | SUB1 | −0.3105 | 0.195 | −0.0722 | 0.164 | 0.2893 | −0.2601 | 0.4595 |
| | RAB11FIP1 | 0.2672 | 0.2596 | 0.417 | 0.0409 | −0.1805 | −0.1013 | −0.4698 |
| | USO1 | 0.2686 | −0.1452 | −0.2772 | −0.4143 | −0.0122 | −0.0026 | −0.2042 |
| | NIPSNAP3A | −0.3867 | −0.1014 | −0.2274 | 0.2312 | −0.4291 | −0.4291 | −0.4291 |
| | ANAPC13 | −0.3301 | −0.1456 | −0.4593 | −0.4593 | −0.3382 | −0.3614 | −0.0778 |
| | AEN | 0.1761 | −0.0846 | 0.0012 | −0.0273 | −0.3173 | −0.0436 | −0.1685 |
| | SF3B4 | 0.2912 | 0.3817 | 0.0442 | 0.3727 | −0.3073 | −0.1565 | −0.0781 |
| | CAV1 | −0.2455 | −0.403 | 0.3843 | 0.3843 | −0.0859 | −0.0859 | −0.0859 |
| | PSPC1 | −0.0614 | 0.0018 | 0.1769 | 0.1057 | −0.0005 | −0.025 | −0.0018 |
| | TFRC | −0.0527 | 0.0709 | −0.4001 | 0.3541 | −0.2494 | −0.2187 | −0.0524 |
| | WDR48 | −1.00E−04 | 0.0808 | −0.3056 | 0.4526 | −0.2564 | 0.4042 | −0.2266 |
| | INO80C | −0.1883 | −0.2152 | 0.4252 | −0.46 | −0.2165 | −0.2165 | −0.2165 |
| | NOP58 | −1.00E−04 | −0.4415 | 0.3428 | −0.3368 | −0.2936 | −0.2531 | −0.1065 |
| | NFAT5 | −0.0029 | 0.2366 | −0.212 | 0.4044 | −0.4489 | 0.3848 | −0.4302 |
| | LBH | −1.00E−04 | −0.3905 | 0.333 | 0.4471 | 0.1954 | −0.318 | −0.0682 |
| | LMAM2 | −0.002 | 0.4619 | 0.1469 | 0.1646 | 0.104 | 0.2156 | −0.0886 |
| | ACOT9 | −0.0009 | 0.4706 | 0.0326 | 0.2002 | −0.1245 | −0.2871 | −0.109 |
| | BRAP | −0.0406 | 0.0466 | 0.0074 | 0.0555 | −0.3942 | −0.1341 | 0.4583 |
| | SLC7A5 | −0.0066 | 0.0137 | 0.1284 | 0.3815 | −0.2993 | −0.0871 | 0.2222 |
| | CCT5 | −0.0567 | 0.2944 | 0.2936 | −0.3304 | −0.3646 | −0.1422 | −0.3248 |
| | NAT10 | −0.3854 | 0.0142 | 0.2479 | 0.1495 | −0.1787 | −0.1792 | −0.1787 |
| | YBX1 | −0.1274 | −0.4899 | 0.2561 | 0.0616 | 0.4804 | −0.3283 | −0.3269 |
| | IMPDH2 | −0.131 | 0.2574 | 0.345 | 0.1184 | −0.1632 | −0.1183 | −0.3474 |
| | PPM1B | −0.0159 | 0.0173 | 0.0417 | 0.0586 | −0.0138 | −0.0005 | −0.0009 |
| | BANF1 | −0.106 | 0.1244 | 0.2802 | 0.0364 | −0.027 | −0.0016 | −0.075 |
| | PLEKHO2 | −0.04 | 0.1219 | 0.064 | 0.0351 | −0.0157 | −0.1339 | −0.1227 |
| | HSPBP1 | −0.033 | 0.0524 | 0.1089 | 0.0002 | −0.3442 | −0.1411 | −0.2215 |
| | JTB | −0.1505 | 0.1165 | 0.0001 | 0.012 | 0.1356 | −0.1247 | 0.2886 |
| | SRA1 | −0.0757 | 0.0002 | 0.0001 | 0.0025 | 0.3843 | 0.1074 | −0.3749 |
| | METTL9 | −0.0081 | 0.0027 | 0.0345 | 0.0185 | 0.212 | 0.4737 | 0.212 |
| | SLC44A2 | −0.0096 | 0.1635 | 0.0139 | 0.0071 | 0.1252 | 0.2252 | 0.0493 |
| | MYCBP | −0.0008 | 0.3912 | −0.352 | −0.4869 | 0.1103 | 0.0857 | 0.1103 |
| | KIAA0101 | −0.3687 | 0.2826 | 0.2361 | 0.0894 | 0.2392 | 0.2613 | 0.0264 |

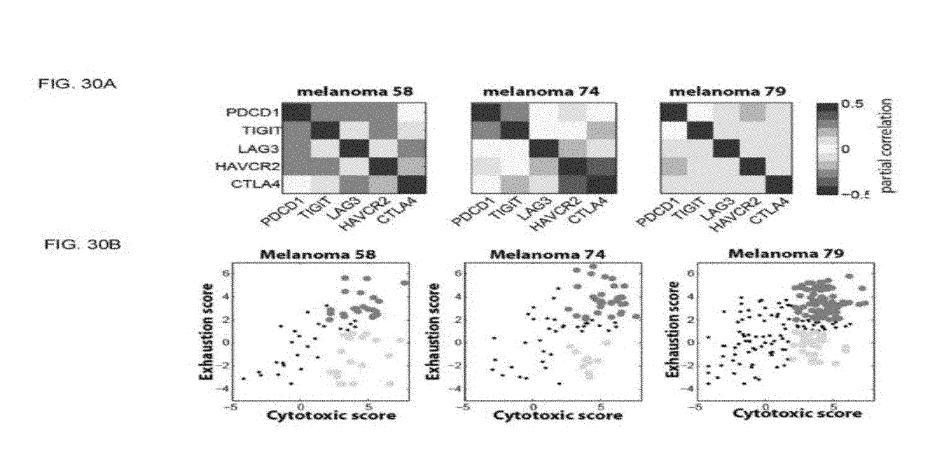

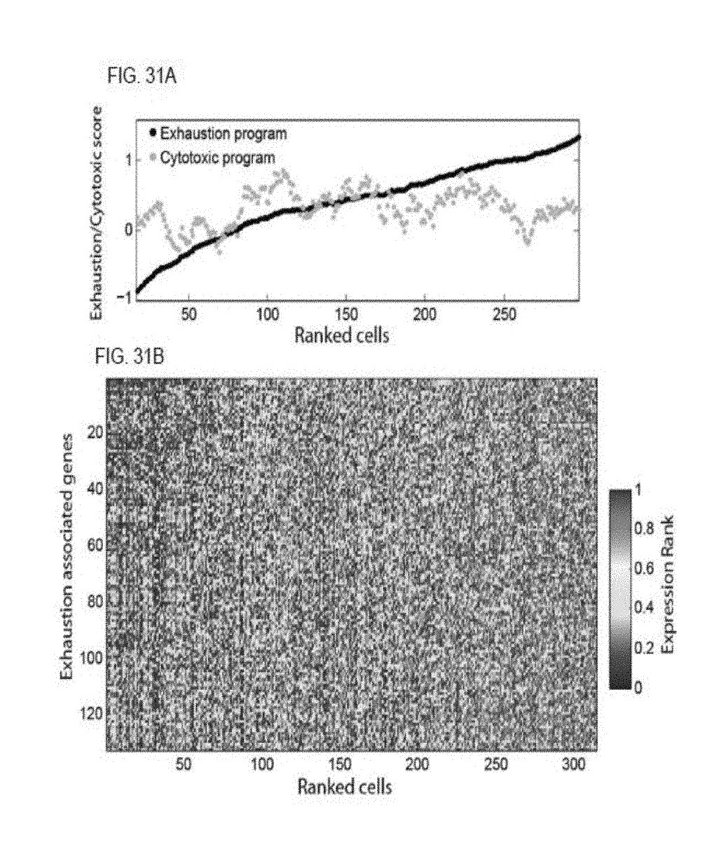

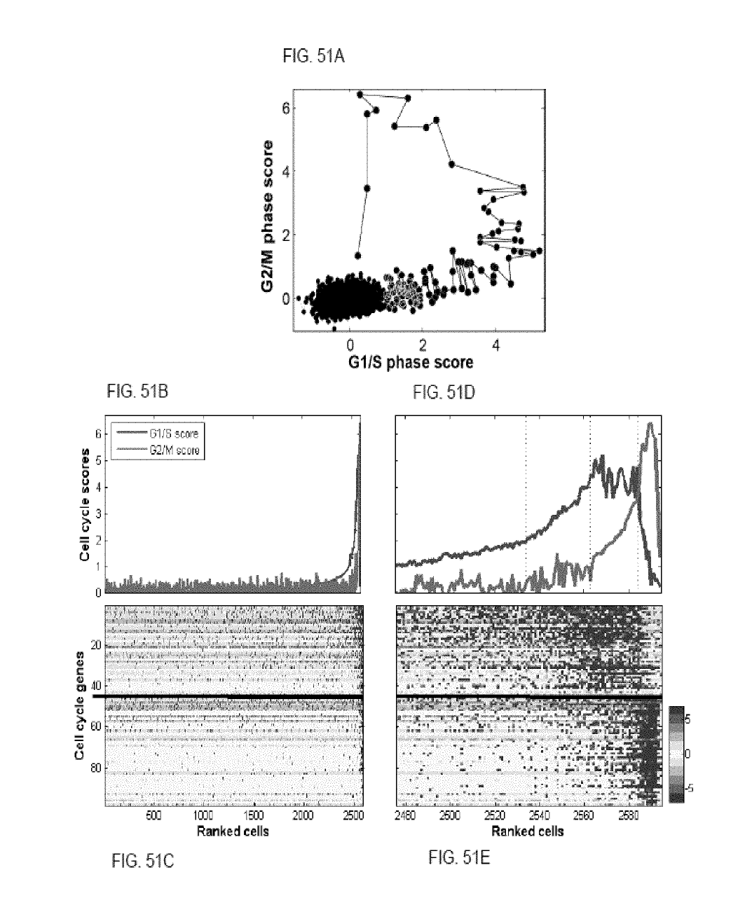

Applicants hypothesized that apart from the co-expression of exhaustion marker genes with cytotoxic marker genes (“activation-dependent exhaustion expression”) the exhaustion genes are also regulated through other mechanisms that may be a better proxy for the exhaustion state of T-cells (“activation-independent exhaustion expression”). Indeed, when restricting the analysis to subsets of cells with comparable cytotoxic gene expression, thereby removing the influence of activation-dependent expression, Applicants still detected significant co-expression among exhaustion markers, which enabled us to define subsets of activation-independent low-exhaustion and high-exhaustion cells in three tumors (FIG. 51 and FIG. 30). These subsets had a similar frequency of cycling cells (FIG. 17), indicating that T-cell exhaustion likely has only a limited effect on proliferation.

A set of 153 genes had significantly higher expression in high-exhaustion compared to low-exhaustion cells in at least one of the three tumors examined. Apart from the five markers that were used to evaluate exhaustion, several additional genes (e.g., SIT1) were associated with exhaustion in two or three tumors (FIG. 5J). However, most genes (143 of 153 total exhaustion-associated genes identified) were significantly associated with exhaustion in only one tumor (FIG. 5K), suggesting that distinct functional states are associated with exhaustion in different tumors. These included several T-cell regulatory genes such as SIRPG and CBLB in melanoma 58 and SLA and CD27 in melanoma 74. Such states could possibly reflect the effects of previous treatments on T-cell functional states. While Applicants cannot systematically address this possibility due to the small number of tumors where exhaustion programs could be evaluated, Applicants note that melanoma 58, derived from a patient who developed resistance to CTLA-4 inhibition, had the weakest association of CTLA-4 expression, but a high-exhaustion state. Although different genes were associated with the exhaustion-high subset in each tumor, their overall expression among CD8+ T-cells was similar across the three tumors, indicating that single cell analyses would be required to distinguish these states in other tumors and to explore their connection with functional exhaustion and response to immunotherapies. Together, these results emphasize the putative functional heterogeneity of tumor-infiltrating lymphocytes, and more generally, highlight the utility of single-cell analysis to discover immune cell subtypes that are largely invisible to current immunophenotyping approaches and their molecular underpinning.

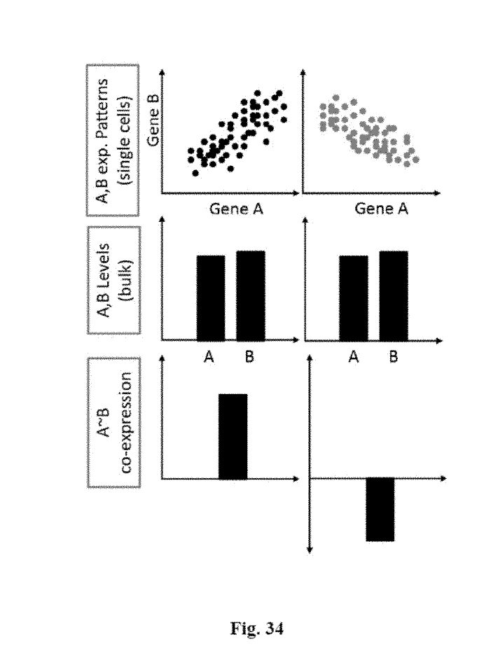

Finally, Applicants explored the relationship between T cell states and clonal expansion. T cells that recognize tumor antigens may proliferate to generate discemible clonal subpopulations defined by an identical T cell receptor (TCR) sequence (48). To identify potential expanded T cell clones, Applicants used RNA-seq reads that map to the TCR to classify single T cells by their isoforms of the V and J segments of the alpha and beta TCR chains, and searched for enriched combinations of TCR segments. As expected, most observed combinations were found in few cells and were not enriched. However, approximately half of the CD8+ T cells in Mel75 had one of seven enriched combinations identified (FDR=0.005), and thus may represent expanded T cell clones (FIG. 5G, FIG. S23). Interestingly, this putative T cell expansion was also linked to exhaustion (FIG. 5H), such that low-exhaustion T cells were significantly depleted of expanded T cells (TCR clusters with >6 cells) and enriched in non-expanded T cells (TCR clusters with 1-4 cells). In particular, the non-exhausted cytotoxic cells are almost all non-expanded (FIG. 5H). In future studies, single-cell RNA-seq profiling of T cells derived from patient tumors before and after treatment with immune checkpoint inhibitors could directly measure the dynamics of clonal and functional architecture and their associated treatment outcomes. Overall, this analysis suggests that single-cell RNA-seq may allow inference of functionally variable T cell populations that are not detectable with other profiling approaches (FIG. 34). This knowledge may empower future studies of tumor response and resistance to immune checkpoint inhibitors.

Conclusion

Here, Applicants have leveraged single-cell RNA-seq to characterize 4,645 malignant and non-malignant cells of the tumor microenvironment from 19 patient-derived melanomas. The analysis uncovered intra- and inter-individual, spatial, functional and genomic heterogeneity in melanoma cells and associated tumor components that shape the microenvironment, including immune cells, CAFs, and endothelial cells. Applicants identified a cell state in a subpopulation of all melanomas studied that is linked to resistance to targeted therapies and validated the presence of a dormant drug-resistant population in a number of melanoma cell lines using different approaches.

By leveraging single cell profiles from a few tumors to deconvolve a large collection of bulk profiles from TCGA, Applicants discovered different microenvironments that are associated with distinct malignant cell profiles, and a subset of genes expressed by one cell type (e.g., CAFs) that may influence the proportion of cells present of another cell type (e.g., T cells), suggesting the importance of intercellular communication for tumor phenotype. Applicants validated putative interactions between stromal-derived factors and the immune-cell abundance in a large independent set of melanoma core biopsies. These observations suggest that new diagnostic and therapeutic strategies that consider tumor cell composition rather than bulk expression may prove advantageous in the future.

Finally, Applicants dissected putative functional differences between exhausted and cytotoxic T cells—only detectable in the co-variation of the expression of several transcripts directly measurable by single cell RNA-seq—which may serve as biomarkers for immunotherapies, such as immune checkpoint inhibitors.

The present invention advantageously provides the ability to carry out numerous, highly-multiplexed single cell observations within a tumor to provide unprecedented power for identifying meaningful cell subpopulations and gene expression programs that can inform both the analysis of bulk transcriptional data and precision treatment strategies. Single cell genomic profiling enables a deeper understanding of the complex interplay among cells within the tumor ecosystem and its evolution in response to treatment, thereby providing a versatile new tool for future translational applications.

### Example 3—Methods for Glioma

Tumor Dissociation

Patients at the Massachusetts General Hospital were consented preoperatively in all cases according to the Institutional Review Board Protocol 1999P008145. Fresh tumors were collected at time of resection and presence of malignant cells was confirmed by frozen section on adjacent, representative pieces of tissue. Fresh tumor tissue was minced with a scalpel and enzymatically dissociated using a gentle papain-based brain tumor dissociation kit (Miltenyi Biotec). Large pieces of debris were removed with a 100 micron strainer, and dissociated cells were layered carefully onto a 5 mL density gradient (Lympholyte-H, Cedar Lane labs), which was centrifuged at 2,000 rpm for 10 min at room temperature to pellet dead cells and red blood cells. The interface containing live cells was saved and used for staining and flow cytometry. Viability was measured using trypan blue exclusion, which confirmed >90% cell viability.

Fluorescence-Activated Cell Sorting

Primary tumor sorting: Tumor cells were blocked in 1% bovine serum albumin in Hanks buffered saline solution (BSA/HBSS), and then stained first with CD45-Vioblue direct antibody conjugate (Miltenyi Biotec) for 30 min at 4 C. Cells were washed with cold PBS, and then resuspended in 1 mL of BSA/HBSS containing 1 uM calcein AM (Life Technologies) and 0.33 uM TO-PRO-3 iodide (Life Technologies) to co-stain for 30 min before sorting. Fluorescence-activated cell sorting was performed on FACSAria Fusion Special Order System (Becton Dickinson) using 488 nm (calcein AM, 530/30 filter), 640 nm (TO-PRO-3, 670/14 filter), and 405 nm (Vioblue, 450/50 filter) lasers. Fluorescence-minus-one controls were included with all tumors, as well as heat killed controls in early pilot experiments, which were crucial to ensure proper identification of the TO-PRO-3 positive compartment and ensure sorting of the live cell population. Standard, strict forward scatter height versus area criteria were used to discriminate doublets and gate only singlets. Viable cells were identified by staining positive with calcein AM but negative for TO-PRO-3. Single cells were sorted into 96-well plates containing cold buffer TCL buffer (Qiagen) containing 1% beta-mercaptoethanol, snap frozen on dry ice, and then stored at −80C prior to whole transcriptome amplification, library preparation and sequencing. Sorting of cell cultures: The BT54 oligodendroglioma cell line (107) was grown in serum-free conditions [Neurobasal media containing 3 mM glutaMAX, B27 supplement, N2 supplement and penicillin-streptomycin (Life Technologies); 100 ng/mL EGF and 40 ng/mL FGF (R&D Systems). Cells dissociated in TrypLE (ThermoFisher Scientific) were blocked in PBS containing 1% BSA (BSA/PBS), stained for 20 min with CD24-PE direct antibody conjugate (Miltenyi), washed, and resuspended in BSA/PBS containing calcein and TO-PRO-3 to identify live cells as above. Cells in the top and bottom ˜15% of CD24 staining were sorted and cultured in CSC media at a concentration of 20,000 cells per mL in duplicate to monitor spherogenic growth.

Whole Transcriptome Amplification, Library Construction, Sequencing, and Processing

Libraries from isolated single cells were generated based on the Smart-seq2 protocol (Picelli 2014) with the following modifications. RNA from single cells was first purified with Agencourt RNAClean XP beads (Beckman Coulter) prior to oligo-dT primed reverse transcription with Maxima reverse transcriptase and locked TSO oligonucleotide, which was followed by 20 cycle PCR amplification using KAPA HiFi HotStart ReadyMix (KAPA Biosystems) with subsequent Agencourt AMPure XP bead purification as described. Libraries were tagmented using the Nextera XT Library Prep kit (Illumina) with custom barcode adapters (sequences available upon request). Libraries from 384 cells with unique barcodes were combined and sequenced using a NextSeq 500 sequencer (Illumina).

Applicants also analyzed 96 cells from MGH60 with an alternative protocol that incorporates random molecular tags (RMTs, also known us unique molecular identifiers, or UMIs) in order to control for PCR amplification bias, as described previously (119) and obtained similar results.

Paired-end, 38-base reads were mapped to the UCSC hg19 human transcriptome using Bowtie with parameters “-q --phred33-quals -n 1-e 99999999-1 25-I 1-X 2000 -a -m 15 -S -p 6”, which allows alignment of sequences with single base changes such as point mutation in the IDH1 gene. Expression values were calculated from SAM files using RSEM v1.2.3 in paired-end mode using parameters “--estimate-rspd --paired end -sam -p 6”, from which TPM values for each gene were extracted.

Immunohistochemistry

Hematoxylin and eosin and single antibody staining (GFAP, Ki67) was done by the clinical pathology laboratory at the Massachusetts General Hospital per routine protocol. For double GFAP/Ki67 double immunohistochemistry, paraffin-embedded sections were mounted on glass slides, deparaffinized in xylene, treated with 0.5% peroxide in methanol, and rehydrated. Antigen retrieval was done using sodium citrate-based, heat-induced antigen retrieval at pH 6.0. The Dako EnVision G/2 double stain system was used for blocking, staining, and development using rabbit anti-Ki67 antibody (Abcam ab15580 at 1:300) and mouse anti-GFAP antibody (Dako M0761 at 1:100).

RNA In Situ Hybridization

Human tissue was obtained from the Massachusetts General Hospital according to an Institutional Review Board-approved protocol (1999P008145) and informed consent was obtained from all patients. ViewRNA technology (Affymetrix) was used for manual format RNA in situ hybridization. Tissue sections mounted on glass slides were stored at −80 C until ready for hybridization. Slides were baked at 60 C for 1 hour, then denatured at 80 C for 3 min, deparaffinized with Histoclear and ethanol dehydration. RNA targets in dewaxed sections were unmasked by treating with pretreatment buffer at 95 C for 10 min and digested with 1:100 dilution protease at 40 C for 10 min, followed by fixation with 10% formalin for 5 min at room temperature. Probe concentrations were 1:40 for both type 1 (red) and type 6 (blue) probe sets, except that the ApoE probe was used at 1:80 dilution. Probe was incubated on sections for 2 hr at 40 C and then washed serially. Affymetrix Panomics probes included ApoE (type 6, catalogue number VA6-16904 and type 1, catalogue number VA1-18265), OMG (type 1, catalogue number VA1-18161), Sox4 (type 6, catalogue number VA6-18162). CCND2 (type 6, catalogue number VA6-18266). Ki67 (type 1, catalogue number VA1-11033). Signal was amplified using PreAmplifier mix QT for 25 min at 40 C followed by Amplifier mix QT for 15 min at 40 C, and then signal was hybridized with labeled probe at 1:1000 dilution for 15 min at 40 C. Color was developed using Fast Blue substrate for Type 6 probes and Fast Red substrate for Type 1 probes for 30 min at 40 C. Tissue was counterstained with Gill's hematoxylin for 25 sec at room temperature followed by mounting with ADVANTAGE mounting media (Innovex). For quantification of compartments by ISH, at least 1,000 cells were counted in representative areas of the tumors.

Fluorescent In Situ Hybridization (FISH)

The probes used in this study consisted of centromeric (CEP) and locus-specific identifiers (LSI) probes. CEP probes included: CEP2 (2p11.1-q11.1, spectrum orange), CEP4 (4p11-q11, spectrum aqua), CEP9 (9p11-q11, spectrum aqua), CEP12 (12p11.1-q11, spectrum green), CEP17 (17p11.1-q11.1, spectrum aqua) and Y (Yp11.1-q11.1, spectrum green) all obtained from Abbott Molecular. Inc. (Des Plaines, Ill.). LSI probes were 1p36/1q25 and 19q13/19p13 dual-color probe set (Abbott), and bacterial artificial chromosome RP11-351D16 (10q11.21, spectrum red or green; CHORI, Oakland, Calif.).

FISH was performed as described previously (120). Briefly, 5-μm sections of formalin-fixed, paraffin-embedded tumor material were deparaffinized, hydrated, and pretreated with 0.1% pepsin for 1 hour. Slides were then washed in 2× saline-sodium citrate buffer (SSC), dehydrated, air dried, and co-denatured at 80° C. for 5 minutes with a three-color probe panel and hybridized at 37° C. overnight using the Hybrite Hybridization System (Abbott). Two 2 min posthybridization washes were performed in 2×SSC/0.3% NP40 at 72° C. followed by one 1 min wash in 2×SSC at room temperature. Slides were mounted with Vectashield containing 4′,6-diamidino-2-phenylindole (Vector, Burlingame, Calif., USA). Entire sections were observed with an Olympus BX61 fluorescent microscope equipped with a charge-coupled device camera and analysed with Cytovision software (Applied Imaging, Santa Clara, Calif.).

Human NPC Culturing

Human NPCs were dissociated from the subventricular zone of 19 week fetal tissue and resulting neurospheres were expanded as previously described in a 50/50 mixture of DMEM/F12 and Neurobasal A (Invitrogen), supplemented with B27 lacking vitamin A, EGF, FGF, and heparin. Single live NPCs were isolated by FACS from a passage 8 culture and sorted into 96 well plates containing Buffer TCL (Qiagen)+1% beta-mercaptoethanol. For differentiation assays, NPCs were plated in chamber slides coated with poly-d-lysine and laminin, and proliferation media was exchanged over a period of 3 days with base media supplemented with either 1% FBS, 1% FBS+60 ng/mL T3, or FBS+100 nM trans-retinoic acid and 10 ng/mL NT3. Multipotency was confirmed by indirect immunofluorescence after 7 days of differentiation with GFAP (Abcam ab53554), Olig2 (Millipore AB9610), and Neurofilament (Aves).

Single Cell RNA-Seq Data Processing

Expression levels were quantified as E_{i,j}=log₂(TPM/10+1), where TPM_{i,j} refers to transcript-per-million for gene i in sample j, as calculated by RSEM (60). TPM values are divided by 10 since Applicants estimate the complexity of single cell libraries in the order of 100,000 transcripts and would like to avoid counting each transcript ˜10 times, as would be the case with TPM, which may inflate the difference between the expression level of a gene in cells in which the gene is detected and those in which it is not detected.

For each cell, Applicants quantified two quality measures: the number of genes for which at least one read was mapped, and the average expression level of a curated list of housekeeping genes. Applicants then conservatively excluded all cells with either fewer than 3,000 detected genes or an average housekeeping expression (E, as defined above) below 2.5. For the remaining cells Applicants calculated the aggregate expression of each gene as log₂(average(TPM_{i,l . . . n})+), and excluded genes with an aggregate expression below 4, leaving a set of 8008 analyzed genes. For the remaining cells and genes. Applicants defined relative expression by centering the expression levels, Er_{i,j}=E_{i,j}-average[E_{i,l . . . n}]. Centering was performed within each tumor separately in order to decrease the impact of inter-tumoral variability on the combined analysis of the three tumors.

CNV Estimation

Initial CNVs (CNV₀) were estimated by sorting the analyzed genes by their chromosomal location and applying a moving average to the relative expression values, with a sliding window of 100 genes within each chromosome, as previously described (15). To avoid considerable impact of any particular gene on the moving average Applicants limited the relative expression values to [−3,3] by replacing all values above 3 by 3, and replacing values below −3 by −3. This was performed only in the context of CNV estimation. For visualization purposes, in order to include the two chromosomes with fewest analyzed genes (chromosome 18 and 21 with 105 and 75 genes, respectively) Applicants extended the moving average to include up to 50 genes from the flanking chromosomes (e.g. the first window in chromosome 18 consisted of the last 50 genes of chromosome 17 and the first 50 genes of chromosome 18, while the 51 through 56 windows in that chromosome consisted only of chromosome 18 genes). This initial analysis is based on the average expression of genes in each cell compared to the other cells and therefore does not have a proper reference which is required to define the baseline. However, Applicants detected a cluster of cells that have higher values at chromosome 1p and 19q, which Applicants know are deleted in the three tumors, and that have consistent “CNV patterns” across the genome despite the fact that they originate from all three tumors. Applicants thus defined these as the normal cells and used the average CNV estimate at each gene across the normal cells as the baseline. The normal cells included both microglia and oligodendrocytes, which differed in gene expression patterns and therefore also in CNV estimates (e.g. the MHC region in chromosome 6 had consistently higher values in microglia than in oligodendrocytes and cancer cells). Applicants therefore defined two baselines, as the average of all microglia and the average of all oligodendrocytes, and based on these the maximal (BaseMax) and minimal (BaseMin) baseline at each window. The final CNV estimate of cell i at position j was defined as:

$${CNV}_{f} ( i , j ) = \{ \begin{matrix}{CNV}_{0} ( i , j ) - BaseMax ( j ) , if {CNV}_{0} ( i , j ) > BaseMax ( j ) + 0.2 \\ {CNV}_{0} ( i , j ) - BaseMin ( j ) , if {CNV}_{0} ( i , j ) < BaseMin ( j ) - 0.2 \\ 0 , if BaseMin ( j ) - 0.2 < {CNV}_{0} ( i , j ) < BaseMin ( j ) + 0.2\end{matrix}$$

Principal Component Analysis

Applicants performed principal component analysis (PCA) for the relative expression values of all cancer cells (as defined by CNV analysis) from the three tumors combined. The covariance matrix used for PCA was generated using an approach outlined in Shalek et al. (61) to decrease the weight of less reliable “missing” values in the data. The basis of this approach is that due to the limited sensitivity of single cell RNA-seq many genes are not detected in particular cells despite being expressed. This is particularly pronounced for genes that are more lowly expressed, and for cells that have lower library complexity (i.e., for which relatively few genes are detected), and results in non-random patterns in the data, whereby cells may cluster based on their complexity and genes may cluster based on their expression levels, rather than “true” co-variation. To mitigate this effect Applicants assign weights to missing values, such that the weight of E, is proportional to the expectation that gene i will be detected in cell j given the average expression of gene i and the total complexity (number of detected genes) of cell j.

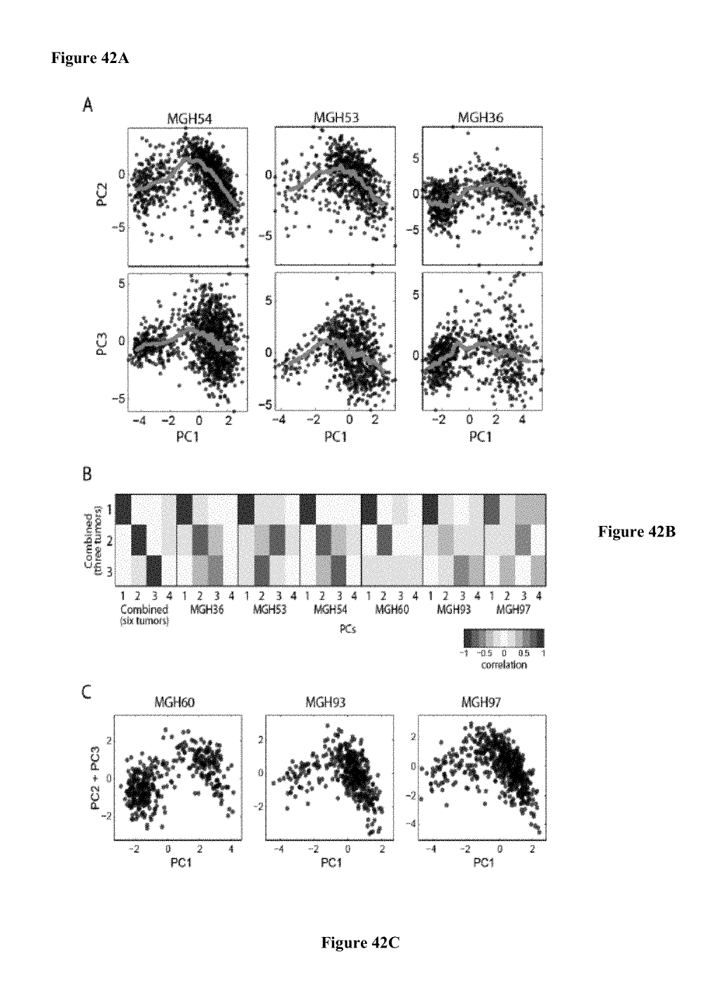

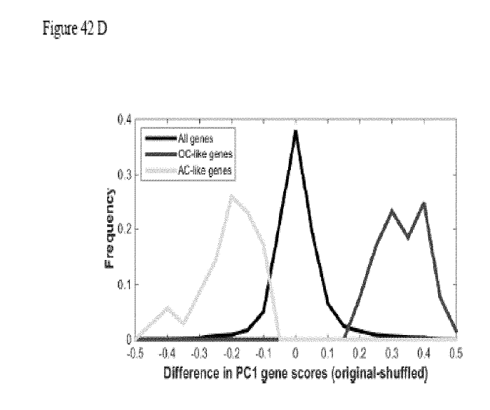

To further verify that the PCA results are not driven by library complexity Applicants compared the PCA results to those of shuffled data. Applicants iteratively swapped the expression of individual genes between pairs of cells with similar complexities, swapping each gene in each cell at least once. In that way Applicants shuffled the data and removed the biological clustering, but maintained the distribution of complexities across cells, as well as the distribution of expression levels for each gene. PCA over the shuffled data defined the complexity-based effect, as evident by a Pearson correlation of 0.96 between the PC1 cell scores and their complexities (in the original data this correlation is only 0.41). Applicants then compared PC gene scores between the original and the shuffled data (FIG. 42D). While PC1 gene scores of most genes are comparable between the two analyses, the loadings of the oligo and astro gene-sets were highly affected. Oligo genes were originally associated with highly positive PC1 scores, and their scores are significantly decreased upon shuffling (97% of the oligodendroglial genes were among the 5% genes with the most decreased loadings, P<10⁻²): similarly, astrocytic genes were originally associated with negative PC1 scores, and their scores are significantly increased upon shuffling (all astrocytic genes were among the 5% genes with most increased loadings. P<10⁻³²). As a result, none of the genes with highest and lowest PC1 scores (after shuffling) overlap with our oligodendroglial and astrocytic gene-sets. Thus, complexity does not account for the association of PC1 with the differentiation programs. Similarly, complexity clearly does not account for the PC2/3 sternness program, as PC2 cell scores are positively correlated with complexity (R=0.27), while PC3 cell scores are negatively correlated with complexity (R=−0.24) and sternness genes were defined as those associated with both PC2 and PC3.

PC1-Associated Genes and Lineage Scores

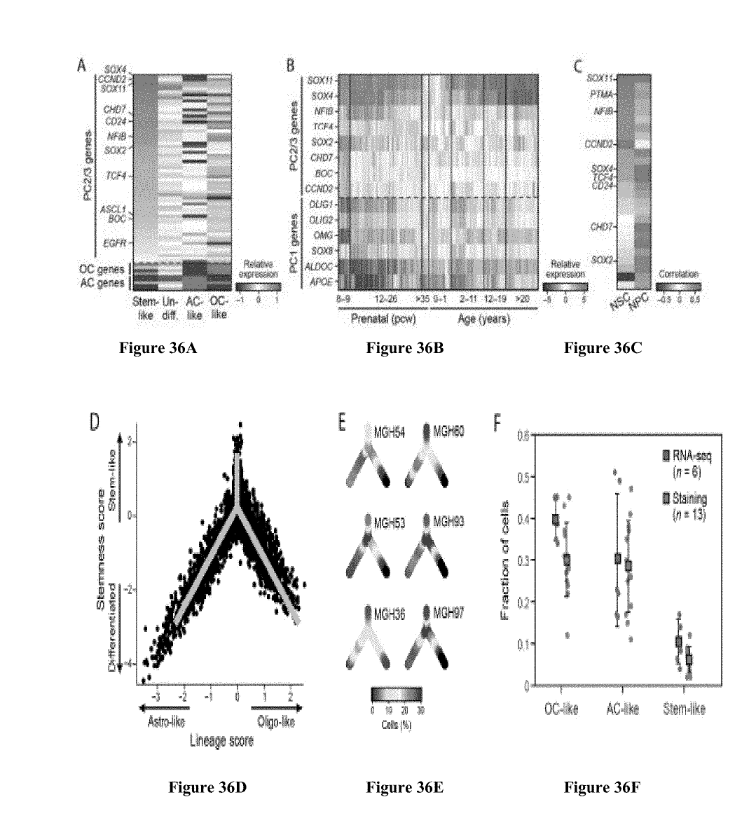

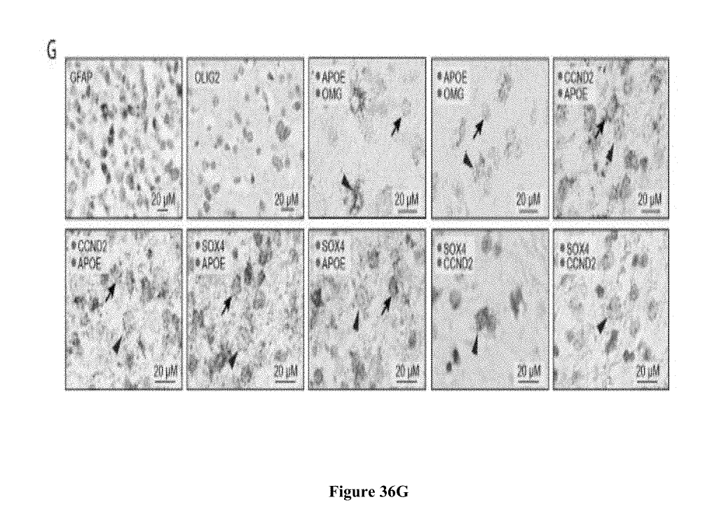

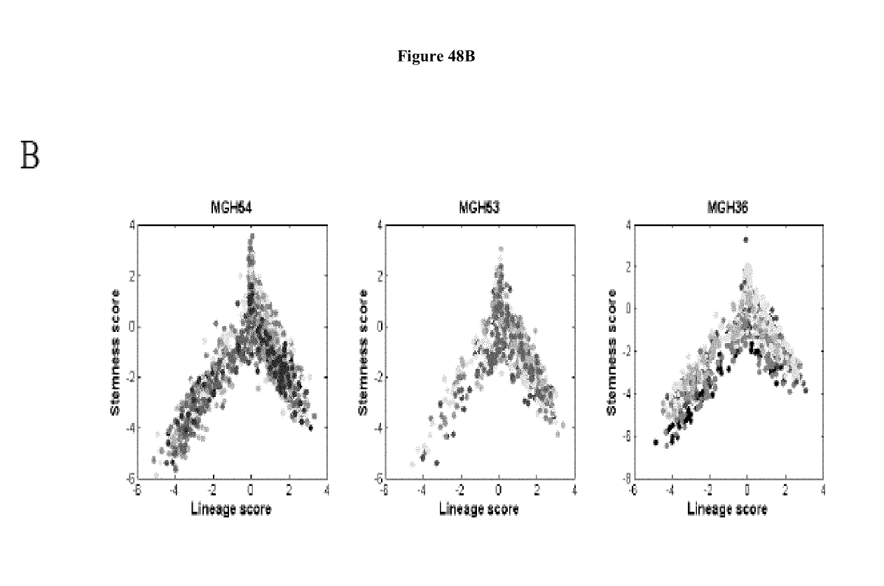

The top correlated genes with PC1 scores (across all tumor cells) were defined as PC1-associated genes. Applicants focused on the genes with an absolute correlation value above 0.35, but note that other thresholds gave similar results (not shown). Of those genes, the subset that was differentially expressed by at least 3-fold between OC and AC mouse cells (97), and for which the two comparisons were consistent (i.e., PC1-positively correlated genes with higher OC expression, and PC1-negatively correlated genes with higher AC expression) were defined as the OC and AC lineage gene-sets. Lineage scores were then calculated as the average relative expression of the lineage gene-set minus the average relative expression of a control gene-set, i.e. Lin_{i,j}=average[Er(Gⱼ,i)]−average[Er(Gⱼ^{cont},i)], where Lin_{i,j} is the score of cell i to lineage j, G, is the gene-set for lineage j and Gⱼ^{cont} is a control gene-set for lineage j. The control gene-set was defined by first binning all 8008 analyzed genes into 25 bins of aggregate expression levels and then, for each gene in the lineage gene-set, randomly select 100) genes from the same expression bin. In this way, the control gene-set has a comparable distribution of expression levels to that of the lineage gene-set and the control gene set is 100-fold larger, such that its average expression is analogous to averaging over 100 randomly-selected gene-sets of the same size as the lineage gene-set. The final lineage score of each cell was defined as the maximal score over the two lineages, LINᵢ=max(Linᵢ OC, LinᵢAC). For visualization purposes in FIG. 36, 37, 38 and in FIGS. 48, 49 and 55 where the two lineage scores are shown in a single axis, Applicants first assigned random scores within [0-0.15] to all cells with LIN<0, to avoid having many overlapping cells at X=0. Second, Applicants assigned negative scores to the cells with higher AC than OC scores (i.e. a cell with AC and OC scores of 0.1 and 1, respectively would be assigned a lineage score of −1 while a cell with AC and OC scores of 1 and 0.1 would be assigned a lineage score of 1).

PC2,3-Associated Genes and Sternness Scores

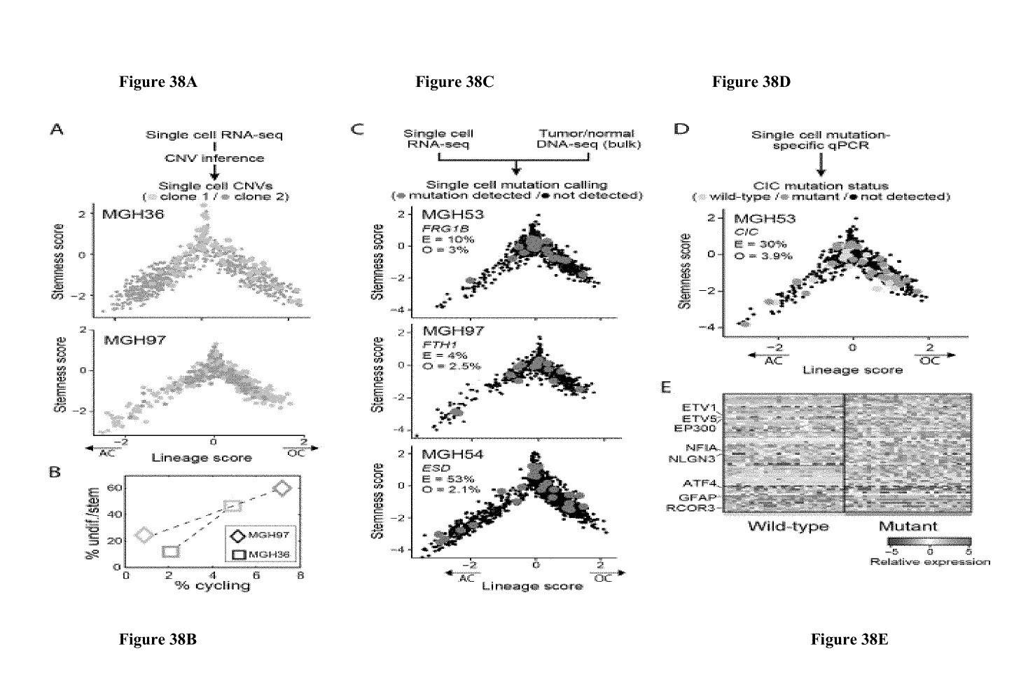

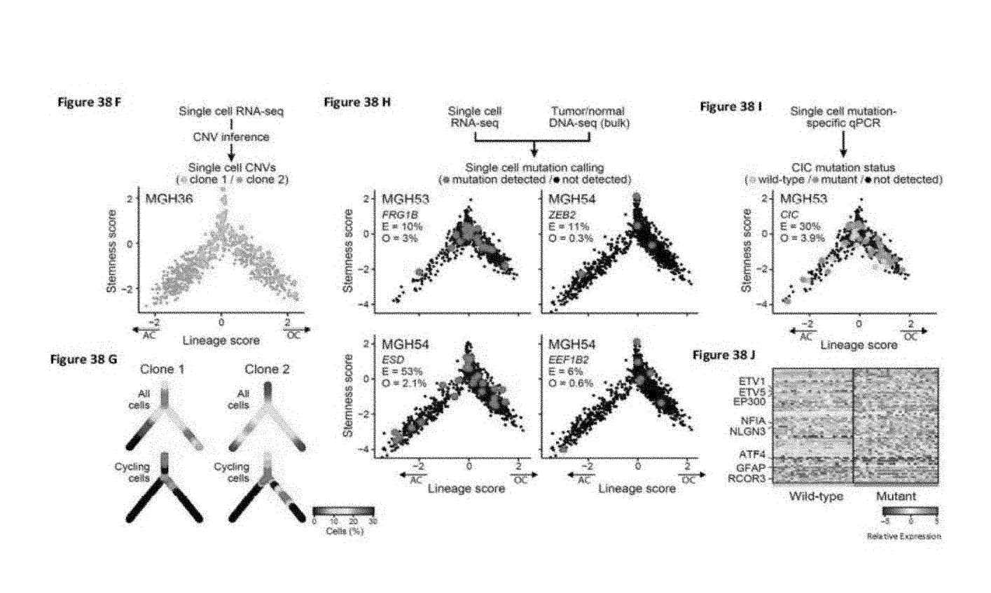

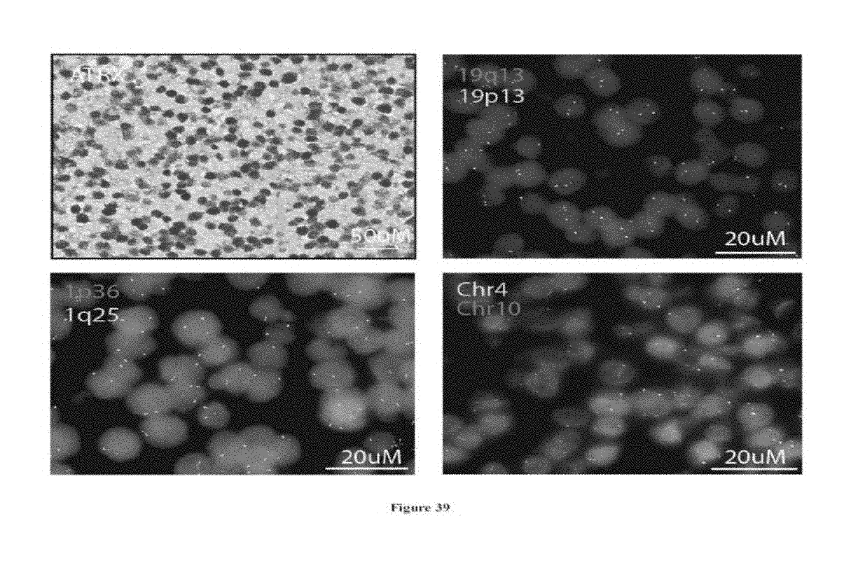

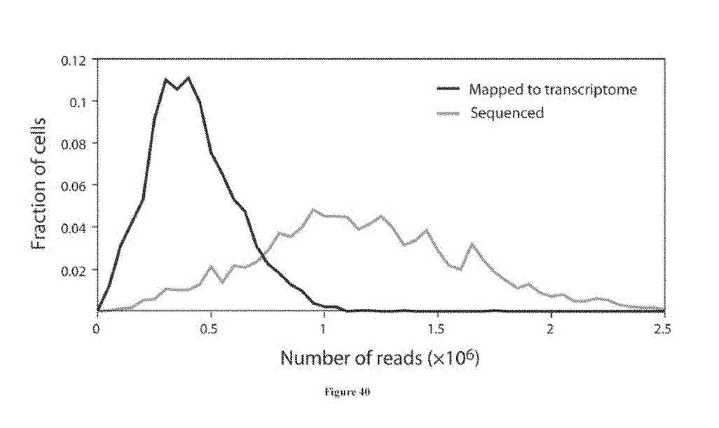

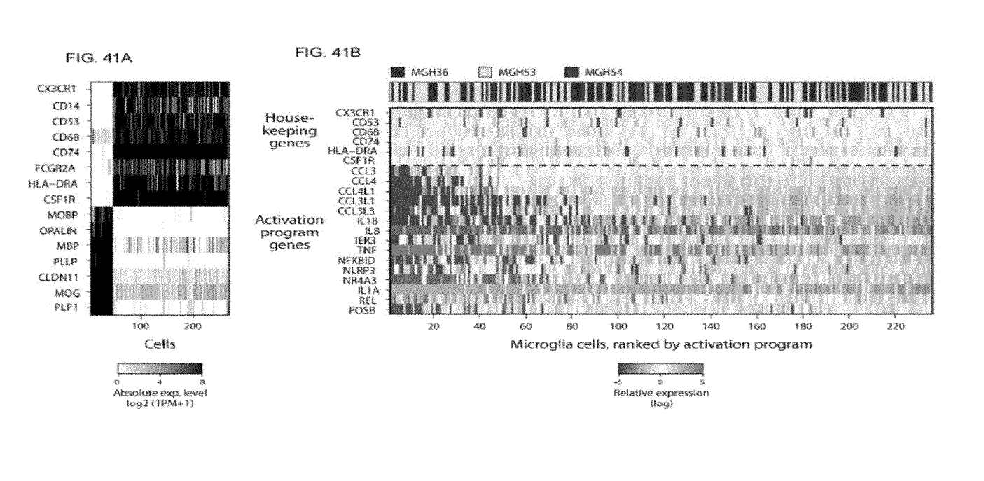

Both PC2 and PC3 were associated with intermediate values of PC1 (FIG. 38) and therefore with presumably less differentiated cells, and Applicants considered their sum as a potential stemness program. To detect potential stem-related genes Applicants chose the top 100 most positively correlated genes with PC2+PC3 scores across all cancer cells from the three tumors. The 100 candidate genes were then restricted to (1) genes that are positively correlated with both PC2 and PC3, which primarily excluded ribosomal protein genes that were only correlated with PC2: (2) genes for which the average relative expression among the stem-like cells (top third of cells by PC2+PC3 scores with a zero lineage score) was above average. Sternness scores for each cell, stem(i), were then defined as the average relative expression of the stemness gene-set (Gₛₜₑₘ) minus the average of a control gene set (Gₛₜₑₘ^{cont}) and minus the lineage score of cell i:

Stem(i)=average[Er(Gₛₜₑₘ)]−average[Er(Gₛₜₑₘ^{cont})]−LIN(i)

Assignment of Cells to Four Subpopulations: Stem/Progenitor-Like, Undifferentiated, OC-Like and AC-Like

Cells were scored for the three programs defined above (two lineage scores and a stemness score) and assigned to the subpopulation that corresponds to their highest scoring program, if the maximal score was above 0.5 and was higher by 0.5 than the score for the other programs. Cells in which the maximal score did not pass these thresholds were assigned to the undifferentiated subpopulation, for which Applicants did not detect a specific expression program. Applicants note that the expression programs are continuous and thus it is difficult to assign all cells to discrete subpopulations. Nevertheless, most cells are highly biased towards one of the three states, and the overall estimates are consistent between analysis of single cell RNA-seq data and tissue staining experiments (FIG. 36f, Table 20). Furthermore, very few cells (˜1% on average, and 5% at most) scored for two programs simultaneously (with the same threshold of 0.5 and no additional criteria, Table 20), with an average frequency of ˜1% of and a maximal frequency of ˜5% cells across the different combinations of programs and different tumors.

Cell Cycle Analysis

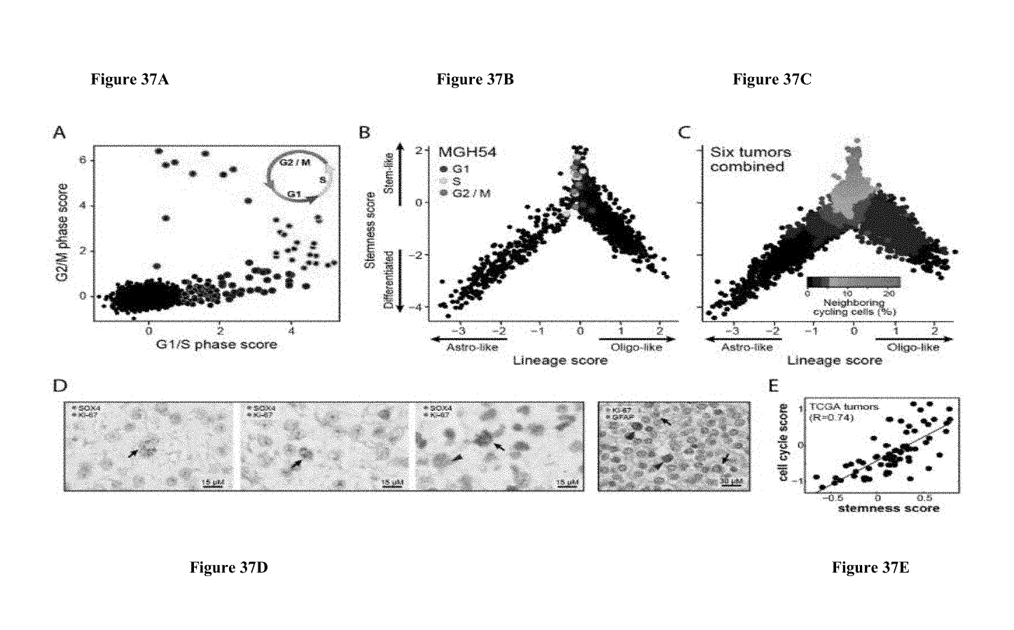

Analysis of single-cell RNA-seq in human (293T) and mouse (3T3) cell lines (16), and in mouse hematopoietic stem cells (124) revealed in each case two prominent cell cycle expression programs that overlap considerably with genes that are known to function in replication and mitosis, respectively, and that have also been found to be expressed at G1/S phases and G2/M phases, respectively, in bulk samples of synchronized HeLa cells (62). Applicants thus defined a core set of 43 G1/S and 55 G2/M genes that included those genes that were detected in the corresponding expression clusters in all four datasets from the three studies described above (Table 18). As expected, the genes in each of those expression programs were highly co-regulated in a small fraction of the oligodendroglioma cells, such that some cells expressed only the G1/S or the G2/M programs and other cells expressed both programs (FIG. 51). Plotting the average expression of these programs revealed an approximate circle (FIG. 37a and FIG. 51a), which Applicants speculate describes the progression along the cell cycle. While Applicants cannot confidently define the regions that correspond to each phase of the cell cycle in an automatic way, Applicants manually defined four regions in the apparent circle and assigned them to approximate cell cycle phases.

Analysis of Whole-Exome DNA Sequencing Data

Output from Illumina software was processed by the Picard processing pipeline to yield BAM files containing aligned reads (bwa version 0.5.9, to the NCBI Human Reference Genome Build hg19) with well-calibrated quality scores (52, 53). Sample contamination by DNA originating from a different individual was assessed using ContEst57(121). Somatic single nucleotide variations (sSNVs) were then detected using MuTect (55). Following this standard procedure, Applicants filter sSNVs by (1) removing potential DNA oxidation artifacts (122): (2) removing events seen in sequencing data of a large panel of ˜8,000 TCGA normal samples; (3) realigning identified sSNVs with NovoAlign (vww.novocraft.com) and performing an additional iteration of MuTect with the newly aligned BAM files. sSNVs were finally annotated using Oncotator⁶⁰. Sample purity and ploidy, as well as Cancer Cell Fraction (CCF) of identified sSNVs were determined by ABSOLUTE (35). Genome-wide copy-ratio profiles were inferred using CapSeg. Read depth at capture targets in tumor samples was calibrated to estimate copy ratio using the depths observed in a panel of normal genomes. Next, Applicants performed allelic copy analysis using reference and alternate counts at germline heterozygous SNP sites.

Mutation Calling in Single Cells

sSNVs that were identified by WES were examined in single-cell RNA-seq data by the mpileup command of SAMtools (Li, H. et al. Bioinformatics 25; 2078-2079 (2009)). The fraction of cells in which Applicants identified these mutations was, on average, only 1.3% of the expected fraction estimated by ABSOLUTE. This low sensitivity primarily reflects the low coverage of the RNA-seq reads over the transcriptome of single cells. Accordingly, sensitivity was correlated with the expression levels of the genes that harbor the mutations, and reached 20.4% for the top 10% most highly expressed genes. Sensitivity was also affected by heterozygosity and allele-specific expression, since in some heterozygote mutant cells Applicants might only sequence the wild-type allele.

Applicants used a targeted sequencing approach to increase our sensitivity for three specific mutations in MGH54 which were identified by WES but detected in very few cells by single cell RNA-seq. Applicants designed primers flanking these three mutations (in ZEB2, EEF1B2 and DNAJC4), PCR-amplified single cell cDNAs (frozen stocks of product from the pre-amplification reaction of the Smart-seq2 protocol) and sequenced the amplified material. This approach was applied for 1056 cells from MGH54. Mutant cells were defined as those with at least 50 reads that mapped to the mutant allele as defined by WES, and for which the fraction of mutant reads was at least 20% of all reads and 5-fold higher than the overall rate of mutant reads (in order to exclude a low rate of mutant reads due to PCR or sequencing errors). The mutations detected by this criteria were highly consistent with those identified from single cell RNA-seq (P<10⁻⁵, hypergeometric test) and uncovered 19 additional mutant calls (three for ZEB2, three for EEF1B2 and 13 for DNAJC4).

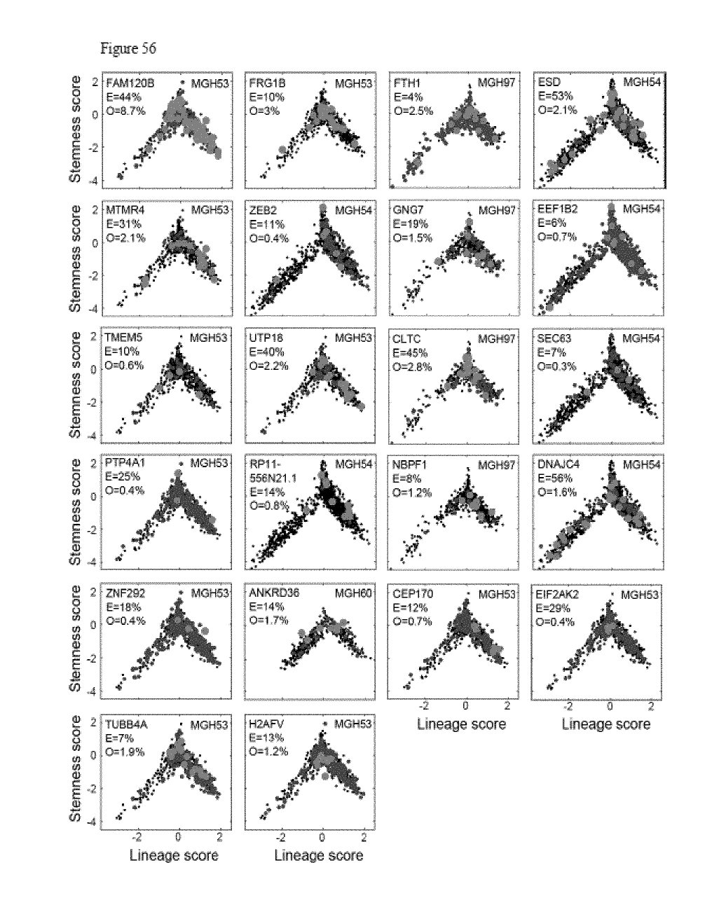

Applicants next focused on the 23 subclonal mutations for which (1) the estimated clonal fraction by ABSOLUTE was at most 60%; (2) at least three cells were identified as harboring the mutation; and (3) at least one cell was identified as having a wild-type allele of the mutant gene. For each of those 19 mutations Applicants plotted the lineage and stemness scores of all mutant cells to examine their distribution of expression states (FIG. 38 and FIG. 56). Note that for these 19 mutations Applicants detected on average 9.4% of the expected fraction by ABSOLUTE.

To estimate the frequency of false-positive errors Applicants defined, for each mutation that is detected by WES and analyzed by RNA-seq mutation calling, (i) “expected mutations”: the number of events in which Applicants find the exact mutation reported by WES, and (ii) “false mutations”: the number of events in which Applicants find a mismatch in the same exact site but to a different base than expected by WES (there are 2 such possible bases). This approach focuses on the exact genomic context of the real mutations to obtain a reliable estimate of the false positive rate. This estimate is half the number of false mutations divided by the number of expected mutations (given 4 bases, one of which is the WT, there are two type of “false mutations” but only one type of “expected mutations”). The result of this analysis was an estimated false positive rate of 0.85%, suggesting that the confidence of each detected mutation is higher than 99%. Accordingly, even in the most extreme case (e.g. ZEB2) where only a single mutant cell is detected in one of the compartments of the hierarchy, Applicants still have a 99% confidence that the mutation is represented in that compartment.

Mutation-Detecting qPCR and Analysis of CIC Mutations

To detect CIC mutations in single cells from MGH53, Applicants performed qPCR using SuperSelective PCR primers, which are highly specific to single base changes due to a loop-out sequence adjacent to the mutant base (legacy.labroots.com/user/webinars/details/id/95). The following qPCR primers were designed to target the c.4543 C>T, p.1515 R>C mutation on CIC cDNA which had been identified as subclonal in MGH53 via whole exome sequencing analysis:

Wild-type-specific forward:
(SEQ ID NO: 20)
5′-CCCTCCAAGGTTTGTCTGCAGccattcGAGGTGC-3′
Mutant-specific forward:
(SEQ ID NO: 21)
5′-CCCTCCAAGGTTTGTCTGCAGccattcGAGGTGT-3′
Universal reverse:
(SEQ ID NO: 22)
5′-tcgGGCAGCCTGCATGATCTT-3′

The specificity of the single cell qPCR primers was validated by two approaches. First, by qPCR on artificial templates differing by only the mutant base. Second, by qPCR on cDNA of single MGH53 tumor cells for which RNA-seq already detected mutant or wild-type reads. These positive control reactions were highly consistent between duplicates and with the mutation status as inferred from RNA-seq: qPCR identified 7 out of 7 mutant cells and 12 out of 15 wild-type cells while the remaining three cells had no qPCR signal, and therefore all qPCR signal was consistent with RNA-seq data Applicants also took advantage of the fact that CIC is located on chr19q which is deleted in MGH53 cancer cells and therefore each cell only contains one CIC allele (loss-of-heterozygosity, LOH). Thus, in a single MGH53 cancer cell, Applicants expect evidence of either mutant or wild-type CIC, but not both. Indeed, all cells with a signal in the positive control assay showed difference in Ct of at least 5 between mutant and wild-type reactions, consistent with LOH.

cDNA was taken from frozen stocks of product from the preamplification reaction of the Smartseq2 protocol. 1 μl from each well of cDNA was used as template for a second round of Smartseq2 preamplification and bead purification in order to increase overall signal downstream. qPCR was performed with the Fast Plus EvaGreen qPCR Master Mix Low Rox (Biotium 31014-1) according to the manufacturer's instructions with the sole modification of adding EDTA to a final reaction concentration of 1.6 mM to enhance primer selectivity. Cp≥33 were considered negative signal; Cp<33 was considered positive signal.

Applicants performed SuperSelective qPCR on cDNA from 467 single MGH53 tumor cells. Of these, 61 cells had signal in both replicates for either mutant or wild type primers, but never for both. These were used to define 28 CIC mutant cells and 27 CIC wild-type cells, after excluding 6 cells which did not pass the single cell RNA-seq QC filters.

To identify genes regulated by the CIC mutation, Applicants compared the 28 CIC mutant and 27 CIC wild-type cells and identified genes with at least 2-fold average expression difference and P<0.01 (before correction for multiple hypothesis testing) based both on a permutation test and a t-test. To further filter the list of differentially expressed genes Applicants also compared the CIC mutant cells to the 671 unresolved cells (in which Applicants did not detect signal for either mutant or wild-type alleles by qPCR and by RNA-seq). Since the fraction of CIC mutants was estimated as 30% by ABSOLUTE Applicants expect the unresolved cells to be a mixture of ˜third CIC-mutants and ˜2/3 CIC-wild type cells, and thus CIC-regulated genes should also differ between this mixture and the CIC mutants but to a lower extent; Applicants used a threshold of 1.5-fold difference between the average expression in CIC mutants and in unresolved cells. The resulting set of differentially expressed genes is given in Table 22. Applicants simulated this analysis with 1,000 randomly selected sets of cells (to replace the CIC mutant and CIC wild-type cells) and found an average of only five upregulated genes by the same criteria, suggesting FDR<0.1 for the genes upregulated by CIC mutation.

### Example 4

Using human oligodendrogliomas as a model, Applicants profiled 4,347 single cells from six patient tumors by RNA-seq, reconstructed their transcriptional architecture and related it to genetic mutations. Application of larger scale single-cell profiling in grade II lesions may more definitively unmask developmental hierarchies in brain tumors, because low-grade gliomas are typically well differentiated and driven by a limited number of genetic events. To further limit inter-tumoral heterogeneity, Applicants focused on oligodendroglioma, a major glioma class that remains incurable (91) and is characterized by signature mutations in IDH1/2 and co-deletion of chromosome arms 1p and 19q. Applicants studied six grade II oligodendrogliomas where IDH1 R132H mutation (or IDH2 R172K mutation) and chromosome 1p/19q co-deletion were confirmed and that had not received pre-operative chemotherapy or radiation (Table 17; FIG. 39) (92).

TABLE 17

| | | | | | Clinical | Clinical | Integrated clinical |
|---|---|---|---|---|---|---|---|
| Designation | Age | Gender | Location | Grade | IDH1 result | FISH result | diagnosis |
| MGH36 | 67 | male | Right | WHO II/III | R132H mutation | 1p19q loss | oligodendroglioma, 1p/19q codeleted |
| | | | frontotemporoinsular | | | | |
| MGH53 | 31 | male | Left frontal | WHO II | R132H mutation | 1p19q loss | oligodendroglioma, 1p/19q codeleted |
| MGH54 | 35 | male | Right parietal | WHO II | R132H mutation | 19q loss, | oligodendroglioma, 1p/19q codeleted |
| | | | | | | borderline | |
| | | | | | | 1p loss | |
| MGH60 | 51 | male | Left | WHO II | R132H mutation | 1p19q loss | oligodendroglioma, 1p19q-codeleted |
| | | | frontotemporoinsular | | | | |

VALIDATION COHORT

| | | | | | | | |
|---|---|---|---|---|---|---|---|
| Oligo 1 | 30 | male | Right frontal | WHO II | R132H mutation | 1p19q loss | recurrent oligodendroglioma, |
| | | | | | | | 1p/19q codeleted |
| Oligo 2 | 51 | male | Right occipital | WHO II | R132H mutation | 1p19q loss | oligodendroglioma, 1p/19q codeleted |
| Oligo 3 | 60 | female | Left temporal | WHO III | R132H mutation | 1p19q loss | anaplastic oligodendroglioma, |
| | | | | | | | 1p/19q codeleted |
| Oligo 4 | 63 | male | Left frontal | WHO III | R132H mutation | 1p19q loss | recurrent anaplastic oligodendroglioma, |
| | | | | | | | 1p/19q codeleted |
| Oligo 5 | 65 | female | Left frontal | WHO II | R132H mutation | 1p19q loss | oligodendroglioma, 1p/19q codeleted |
| Oligo 6 | 13 | female | Left frontal | WHO II | R132H mutation | 1p19q loss | oligodendroglioma, 1p/19q codeleted |
| Oligo 7 | 65 | female | Left parietal | WHO III | R132H mutation | 1p19q loss | recurrent anaplastic oligodendroglioma, |
| | | | | | | | 1p/19q codeleted |
| Oligo 8 | 59 | female | Cerebellar vermis | WHO III | R132H mutation | 1p19q loss | recurrent anaplastic oligodendroglioma, |
| | | | | | | | 1p/19q codeleted |
| Oligo 9 | 50 | male | Left frontal | WHO II | R132H mutation | 1p19q loss | oligodendroglioma, 1p/19q codeleted |
| Oligo 10 | 77 | male | Right | WHO II | R132H mutation | 1p19q loss | oligodendroglioma, 1p/19q codeleted |
| | | | frontotemporoinsular | | | | |

Overall, Applicants performed single cell RNA-seq (93) on 5,172 cells at an average depth of ˜1.2 million reads per cell (FIG. 40), resulting in 4,347 cells that passed the quality controls. Three tumors were analyzed more deeply (MGH36, 53, 54; 791-1,229 cells per tumor that passed our quality controls) and three tumors (MGH60, 93 and 97) were profiled at medium depth (430-598 cells).

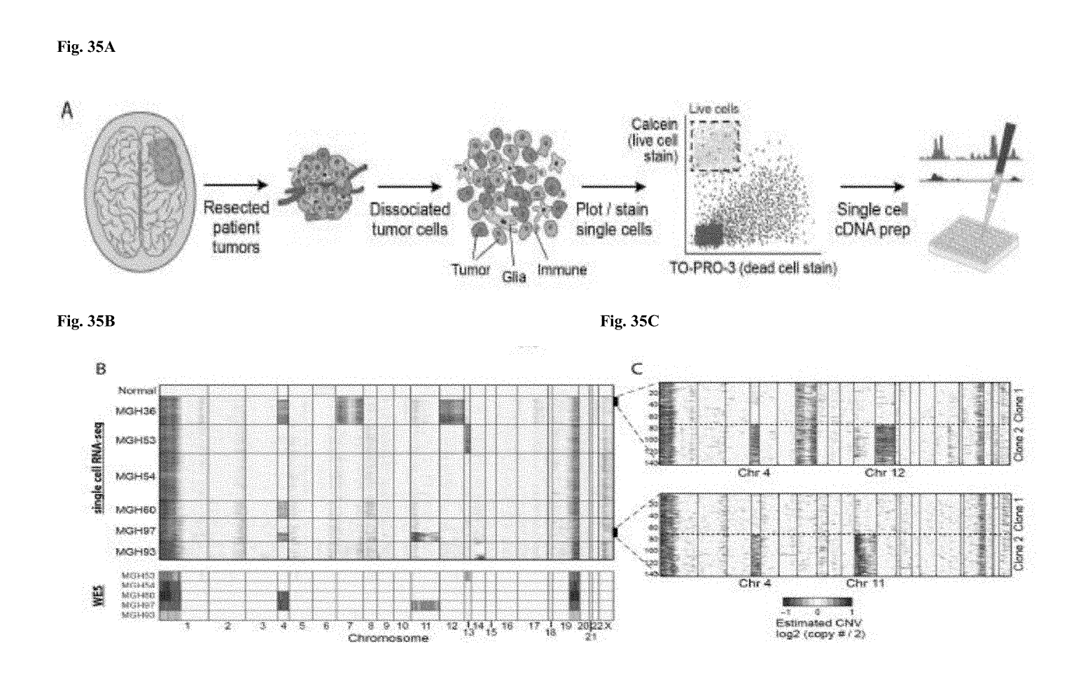

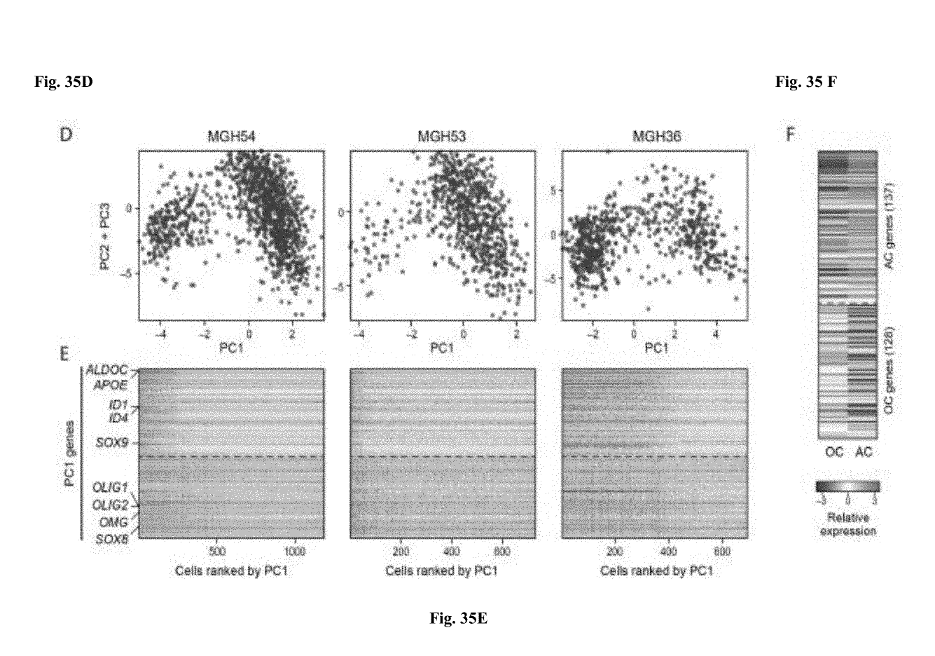

Applicants distinguished malignant from possible non-malignant cells in the tumor microenvironment, by estimating chromosomal copy number variations (CNVs) from the average expression of genes in large chromosomal regions within each cell (FIG. 35b and FIG. 46; Methods) (15). Each tumor contained a large majority of malignant cells with deletions of chromosomes 1p and 19q, the hallmarks of oligodendroglioma, as well as in some cases additional tumor-specific CNVs, which were validated by FISH and by DNA whole-exome sequencing (WES) (FIG. 35b, FIGS. 39 and 46). In two tumors (MGH36, MGH97), CNV analysis pointed to the existence of two clones (FIG. 35b,c) whereby Clone 2 harbored all the CNVs present in Clone 1, as well as additional CNVs, suggesting that Clone 2 was in each case derived through subsequent tumor evolution.

Another 304 cells across the six tumors lacked any detectable CNVs, and clustered by gene expression into two subsets, which differed markedly from the malignant cells and expressed microglia and mature oligodendrocyte markers, respectively, consistent with being non-malignant cell types (FIG. 41a). Applicants detected significant variability between the microglia cells, in which a set of pro-inflammatory cytokines (IL1A/B, IL8 and TNF), chemokines (CCL3/4) and early response genes were coordinately expressed by ˜80% of the microglia (FIG. 41b). This expression program differs from canonical macrophage M1/M2 responses (94) and therefore suggests an unknown microglia expression program that appears to be glioma-specific.

Applicants examined the heterogeneity of the cancer cells from the three tumors for which Applicants analyzed the largest cell numbers by a combined principal component analysis (PCA), while controlling for data quality per transcript and per cell and inter-tumor heterogeneity (Methods). Applicants identified two prominent groups of cells, corresponding to low and high PC1 scores (FIG. 35d) and expressing distinct lineage markers of astrocytes and oligodendrocytes, respectively. These results were highly consistent across all six tumors, and were not simply accounted for by technical and batch effects (Supplementary FIG. 4 and Note 1). Specifically, in each tumor, cells with high PC1 scores were strongly associated with high expression of 137 genes, including markers of oligodendroglial lineage (e.g., OLIG1/2, OMG), and with low expression of 128 genes, including markers of astrocytic lineage (e.g., APOE, ALDOC, SOX9) (FIG. 35e, Table 18) (95). Cells with low PC1 scores had the opposite pattern of expression. Consistent with these specific markers, the orthologs of most PC1-associated genes were preferentially expressed in mice oligodendrocytes (OC) and astrocytes (AC), respectively (FIG. 351) (97). This indicates that oligodendrogliomas are primarily composed of two subpopulations of cells with transcriptional states of distinct glial lineages; this mirrors histopathology, where cancer cells of astrocytic lineage within oligodendrogliomas are known as “microgemistocytes” (98).

TABLE 18
Ranked gene-sets used to define cell cycle, stemness and lineage scores.

| G1/S | G2/M | stemness | AC (PCA-only) | AC (PCA + mice) | OC (PCA-only) | OC (PCA + mice) |
|---|---|---|---|---|---|---|
| MCM5 | HMGB2 | SOX4 | APOE | APOE | LMF1 | OLIG1 |
| PCNA | CDK1 | CCND2 | SPARCL1 | SPARCL1 | OLIG1 | SNX22 |
| TYMS | NUSAP1 | SOX11 | SPOCK1 | ALDOC | SNX22 | GPR17 |
| FEN1 | UBE2C | RBM6 | CRYAB | CLU | POLR2F | DLL3 |
| MCM2 | BIRC5 | HNRNPH1 | ALDOC | EZR | LPPR1 | SOX8 |
| MCM4 | TPX2 | HNRNPL | CLU | SORL1 | GPR17 | NEU4 |
| RRM1 | TOP2A | PTMA | EZR | MLC1 | DLL3 | SLC1A1 |
| UNG | NDC80 | TRA2A | SORL1 | ABCA1 | ANGPTL2 | LIMA1 |
| GINS2 | CKS2 | SET | MLC1 | ATP1B2 | SOX8 | ATCAY |
| MCM6 | NUF2 | C6orf62 | ABCA1 | RGMA | RPS2 | SERINC5 |
| CDCA7 | CKS1B | PTPRS | ATP1B2 | AGT | FERMT1 | LHFPL3 |
| DTL | MKI67 | CHD7 | PAPLN | EEPD1 | PHLDA1 | SIRT2 |
| PRIM1 | TMPO | CD24 | CA12 | CST3 | RPS23 | OMG |
| UHRF1 | CENPF | H3F3B | BBOX1 | SOX9 | NEU4 | APOD |
| MLF1IP | TACC3 | C14orf23 | RGMA | EDNRB | SLC1A1 | MYT1 |
| HELLS | FAM64A | NFIB | AGT | GABRB1 | LIMA1 | OLIG2 |
| RFC2 | SMC4 | SRGAP2C | EEPD1 | PLTP | ATCAY | RTKN |
| RPA2 | CCNB2 | STMN2 | CST3 | JUNB | SERINC5 | FA2H |
| NASP | CKAP2L | SOX2 | SSTR2 | DKK3 | CDH13 | MARCKSL1 |
| RAD51AP1 | CKAP2 | TFDP2 | SOX9 | ID4 | CXADR | LIMS2 |
| GMNN | AURKB | CORO1C | RND3 | ADCYAP1R1 | LHFPL3 | PHLDB1 |
| WDR76 | BUB1 | EIF4B | EDNRB | GLUL | ARL4A | RAB33A |
| SLBP | KIF11 | FBLIM1 | GABRB1 | PFKFB3 | SHD | OPCML |
| CCNE2 | ANP32E | SPDYE7P | PLTP | CPE | RPL31 | SHISA4 |
| UBP7 | TUBB4B | TCF4 | JUNB | ZFP36L1 | GAP43 | TMEFF2 |
| POLD3 | GTSE1 | ORC6 | DKK3 | JUN | IFITM10 | NME1 |
| MSH2 | KIF20B | SPDYE1 | ID4 | SLC1A3 | SIRT2 | NXPH1 |
| ATAD2 | HJURP | NCRUPAR | ADCYAP1R1 | CDC42EP4 | OMG | GRIA4 |
| RAD51 | HJURP | BAZ2B | GLUL | NTRK2 | RGMB | SGK1 |
| RRM2 | CDCA3 | NELL2 | EPAS1 | CBS | HIPK2 | ZDHHC9 |
| CDC45 | HN1 | OPHN1 | PFKFB3 | DOK5 | APOD | CSPG4 |
| CDC6 | CDC20 | SPHKAP | ANLN | FOS | NPPA | LRRN1 |
| EXO1 | TTK | RAB42 | HEPN1 | TRIL | EEF1B2 | BIN1 |
| TIPIN | CDC25C | LOH12CR2 | CPE | SLC1A2 | RPS17L | EBP |
| DSCC1 | KIF2C | ASCL1 | RASL10A | ATP13A4 | FXYD6 | CNP |
| BLM | RANGAP1 | BOC | SEMA6A | ID1 | MYT1 | |
| CASP8AP2 | NCAPD2 | ZBTB8A | ZFP36L1 | TPCN1 | RGR | |
| USP1 | DLGAP5 | ZNF793 | HEY1 | FOSB | OLIG2 | |
| CLSPN | CDCA2 | TOX3 | PRLHR | LIX1 | ZCCHC24 | |
| POLA1 | CDCA8 | EGFR | TACR1 | IL33 | MTSS1 | |
| CHAF1B | ECT2 | PGM5P2 | JUN | TIMP3 | GNB2L1 | |
| BRIP1 | KIF23 | EEF1A1 | GADD45B | NHSL1 | C17orf76-AS1 | |
| E2F8 | HMMR | MALAT1 | SLC1A3 | ZFP36L2 | ACTG1 | |
| | AURKA | TATDN3 | CDC42EP4 | DTNA | EPN2 | |
| | PSRC1 | CCL5 | MMD2 | ARHGEF26 | PGRMC1 | |
| | ANLN | EVI2A | CPNE5 | TBC1D10A | TMSB10 | |
| | LBR | LYZ | CPVL | LHFP | NAP1L1 | |
| | CKAP5 | POU5F1 | RHOB | NOG | EEF2 | |
| | CENPE | FBXO27 | NTRK2 | LCAT | MIAT | |
| | CTCF | CAMK2N1 | CBS | LRIG1 | CDHR1 | |
| | NEK2 | NEK5 | DOK5 | GATSL3 | TRAF4 | |
| | G2E3 | PABPC1 | TOB2 | ACSL6 | TMEM97 | |
| | GAS2L3 | AFMID | FOS | HEPACAM | NACA | |
| | CBX5 | QPCTL | TRIL | SCG3 | RPSAP58 | |
| | CENPA | MBOAT1 | NFKBIA | RFX4 | SCD | |
| | | HAPLN1 | SLC1A2 | NDRG2 | TNK2 | |
| | | LOC90834 | MTHFD2 | HSPB8 | RTKN | |
| | | LRTOMT | IER2 | ATF3 | UQCRB | |
| | | GATM-AS1 | EFEMP1 | PON2 | FA2H | |
| | | AZGP1 | ATP13A4 | ZFP36 | MIF | |
| | | RAMP2-AS1 | KCNIP2 | PER1 | TUBB3 | |
| | | SPDYE5 | ID1 | BTG2 | COX7C | |
| | | TNFAIP8L1 | TPCN1 | NRP1 | AMOTL2 | |
| | | | LRRC8A | PRRT2 | THY1 | |
| | | | MT2A | F3 | NPM1 | |
| | | | FOSB | | MARCKSL1 | |
| | | | L1CAM | | LIMS2 | |
| | | | LIX1 | | PHLDB1 | |
| | | | HLA-E | | RAB33A | |
| | | | PEA15 | | GRIA2 | |
| | | | MT1X | | OPCML | |
| | | | IL33 | | SHISA4 | |
| | | | LPL | | TMEFF2 | |
| | | | IGFBP7 | | ACAT2 | |
| | | | C1orf61 | | HIP1 | |
| | | | FXYD7 | | NME1 | |
| | | | TIMP3 | | NXPH1 | |
| | | | RASSF4 | | FDPS | |
| | | | HNMT | | MAP1A | |
| | | | JUND | | DLL1 | |
| | | | NHSL1 | | TAGLN3 | |
| | | | ZFP36L2 | | PID1 | |
| | | | SRPX | | KLRC2 | |
| | | | DTNA | | AFAP1L2 | |
| | | | ARHGEF26 | | LDHB | |
| | | | SPON1 | | TUBB4A | |
| | | | TBC1D10A | | ASIC1 | |
| | | | DGKG | | TM7SF2 | |
| | | | LHFP | | GRIA4 | |
| | | | FTH1 | | SGK1 | |
| | | | NOG | | P2RX7 | |
| | | | LCAT | | WSCD1 | |
| | | | LRIG1 | | ATP5E | |
| | | | GATSL3 | | ZDHHC9 | |
| | | | EGLN3 | | MAML2 | |
| | | | ACSL6 | | UGT8 | |
| | | | HEPACAM | | C2orf27A | |
| | | | ST6GAL2 | | VIPR2 | |
| | | | KIF21A | | DHCR24 | |
| | | | SCG3 | | NME2 | |
| | | | METTL7A | | TCF12 | |
| | | | CHST9 | | MEST | |
| | | | RFX4 | | CSPG4 | |
| | | | P2RY1 | | GAS5 | |
| | | | ZFAND5 | | MAP2 | |
| | | | TSPAN12 | | LRRN1 | |
| | | | SLC39A11 | | GRIK2 | |
| | | | NDRG2 | | FABP7 | |
| | | | HSPB8 | | EIF3E | |
| | | | IL11RA | | RPL13A | |
| | | | SERPINA3 | | ZEB2 | |
| | | | LYPD1 | | EIF3L | |
| | | | KCNH7 | | BIN1 | |
| | | | ATF3 | | FGFBP3 | |
| | | | TMEM151B | | RAB2A | |
| | | | PSAP | | SNX1 | |
| | | | HIF1A | | KCNIP3 | |
| | | | PON2 | | EBP | |
| | | | HIF3A | | CRB1 | |
| | | | MAFB | | RPS10-NUDT3 | |
| | | | SCG2 | | GPR37L1 | |
| | | | GRIA1 | | CNP | |
| | | | ZFP36 | | DHCR7 | |
| | | | GRAMD3 | | MICAL1 | |
| | | | PER1 | | TUBB | |
| | | | TNS1 | | FAU | |
| | | | BTG2 | | TMSB4X | |
| | | | CASQ1 | | PHACTR3 | |
| | | | GPR75 | | | |
| | | | TSC22D4 | | | |
| | | | NRP1 | | | |
| | | | DNASE2 | | | |
| | | | DAND5 | | | |
| | | | SF3A1 | | | |
| | | | PRRT2 | | | |
| | | | DNAJB1 | | | |
| | | | F3 | | | |

Each gene-set is ranked from most significant (top) to least significant gene (bottom). Significance was determined by average fold-change of upregulation in G1/S, G2/M and stem-like cells (first three columns) or by the correlation with PC1 (positive correlation for OC genes and negative for AC genes).
Two gene-sets are given for each of the lineages:
“PCA-only” denote genes that were identified from PCA analysis of oligodendroglioma cells and are presented in FIG. 35.
“PCA + mice” denote genes that were both idnetified in the PCA analysis of oligodendroglioma cells and are preferentially expressed in the resective lineage in mice (Methods), and these were used to estimate lineage scores.

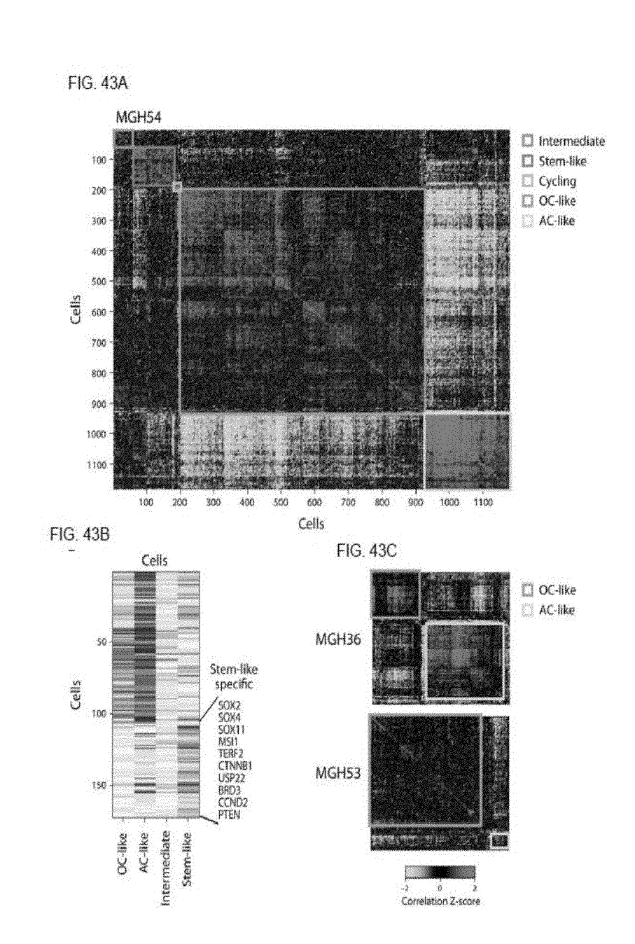

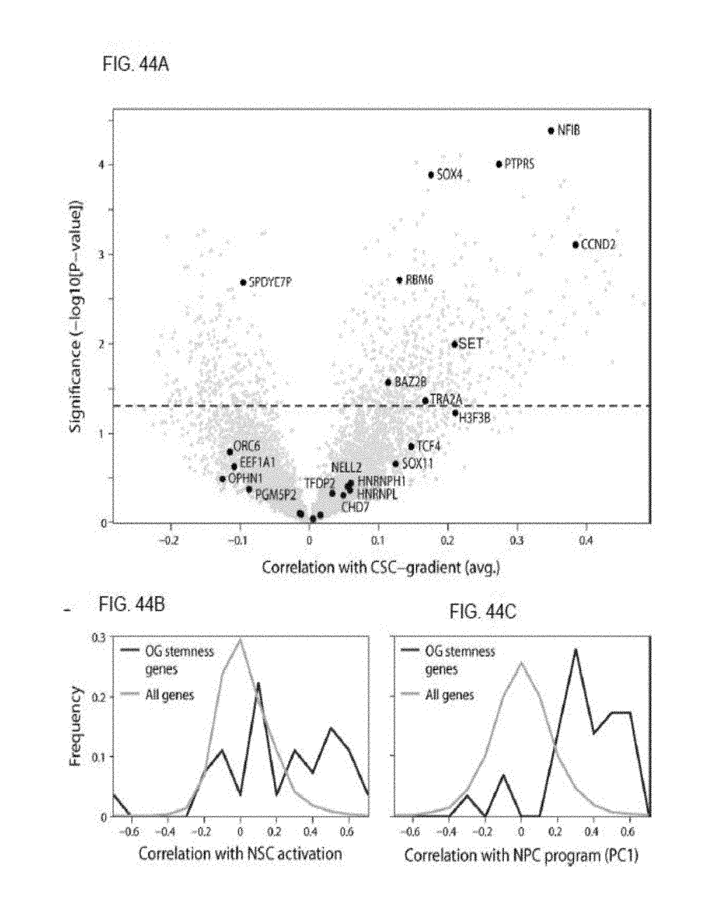

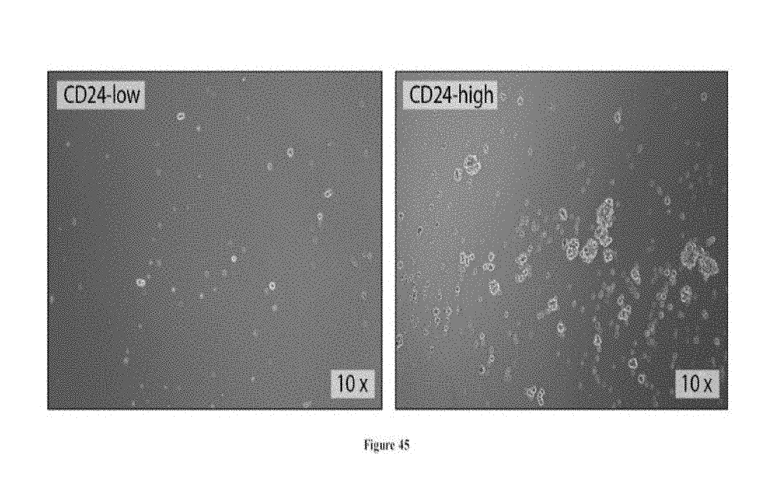

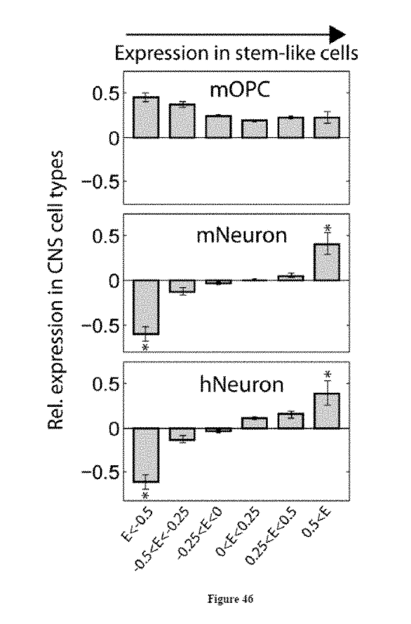

Cells with high PC2 and PC3 scores showed an association with intermediate values of PC1 (shown both for PC2+PC3 (FIG. 35d). (FIG. 42c) and separately for PC2 and for PC3 (FIG. 42a)), indicating a lack of differentiation and prompting us to explore additional programs. (As for PC1, these patterns were not the result of technical or batch effects; Note 1). 63 genes were associated with both PC2 and PC3 (Table 18). Several lines of evidence indicate that this represents a “stemness” program. First, among the 20 highest-ranking genes associated with PC2/3 (FIG. 36a) were SOX4, SOX11 and SOX2, neurodevelopmental transcription factors critical to neural stem cells and self-renewal of glioma stem cells (99-101). Additional genes with important roles in neurogenesis and in the CSC program of gliomas included the transcription factors NFIB and ASCL1, the chromatin remodeler CHD7, the cell surface protein CD24, and BOC and TCF4, which function in signaling pathways central to stem cell maintenance (74, 15, 99-104). Similar results were obtained by hierarchical clustering, showing a distinct cluster of cells that preferentially express these PC2/3-associated stemness regulators (FIG. 43). Second, several genes of this oligodendroglioma “stemness” program were previously identified by our study on single cell RNA-seq in primary human glioblastoma CSC (FIG. 44a, P=1.5*10⁻⁴ for the overlap between the two sternness programs, hypergeometric test), albeit each program also contains specific regulators, such as CD24 which emerged as the top cell surface marker in the oligodendroglioma program. Third, analysis of the human brain transcriptome dataset from the Allen Brain Atlas showed that the expression of PC2/3-associated regulators was highest in early prenatal human brain samples and dropped significantly after birth, in childhood and adult samples, further indicating a role in neural development (FIG. 36b, P=8*10⁻¹⁸ for the enrichment of PC2/3-associated genes in prenatal vs. adult samples, t-test) (105). This pattern was particularly pronounced for SOX4 and for SOX11, which was the gene most significantly enriched in prenatal samples across the human genome (P=4*10⁻⁵⁰, t-test), while an opposite pattern was found for AC and OC lineage genes (FIG. 36b). Similarly, interrogating a recently published study of single-cell RNA-seq analysis of the human brain, Applicants identified several PC2/3-associated genes as preferentially expressed in single-cells in fetal human brain, while Applicants did not identify any adult human brain cell type expressing this signature (P=0.006 for enrichment of PC2/3-associated genes in the fetal vs. adult programs, hypergeometric test) (106). Based on these four lines of evidence, cells with intermediate PC1 values were thus separated into “undifferentiated” (low PC2/3) and “stem/progenitors” (high PC2/3) cells (FIG. 36a).

Oligodendrogliomas are often thought to arise from transformation of oligodendrocyte progenitor cells (OPCs) (108), raising the possibility that the “stem/progenitors” PC2/3 genes may reflect an OPC-like program. However, the PC2/3-associated genes were not preferentially expressed in OPCs; instead, these genes were preferentially expressed in cells of neuronal lineage (FIG. 46) (97, 123). Thus, although oligodendroglioma display only glial differentiation (both molecularly and histologically) and are thought to be derived from glial precursors, they may harbor rare cells that resemble primitive neural stem/progenitor cells that are normally tri-potent, capable of producing both glial lineages as well as neurons; genetic mutations may skew these tri-potent cancer cells towards generating glia (109,110). Consistent with this possibility, most PC2/3-associated genes, including SOX4 and SOX 11, were upregulated upon activation of tri-potent mice neural stem cell (111) (NSCs) (FIG. 36c, FIG. 44b; P=3*10⁻⁶, t-test).

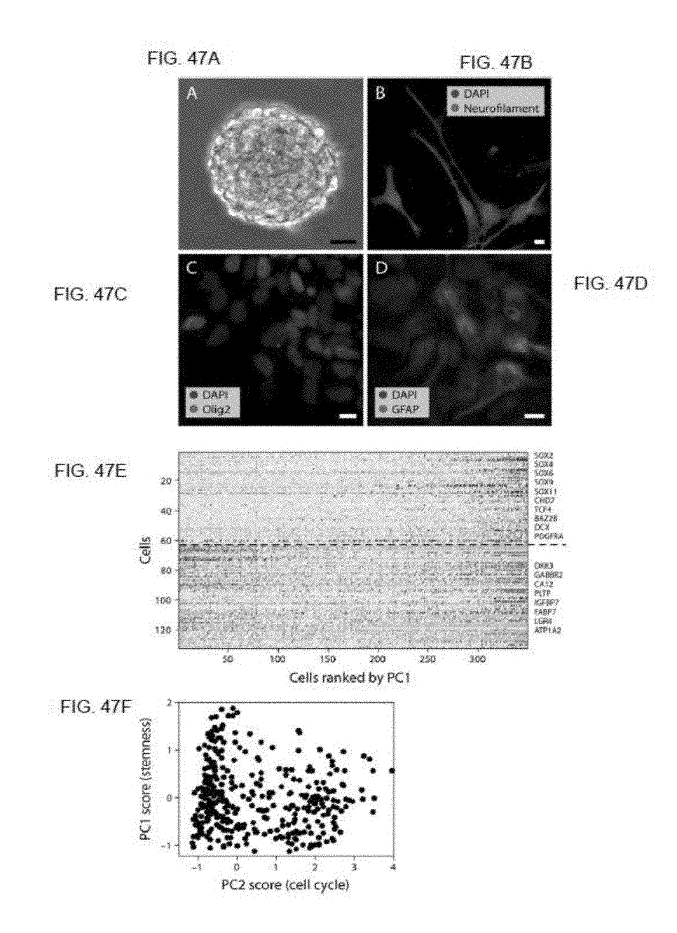

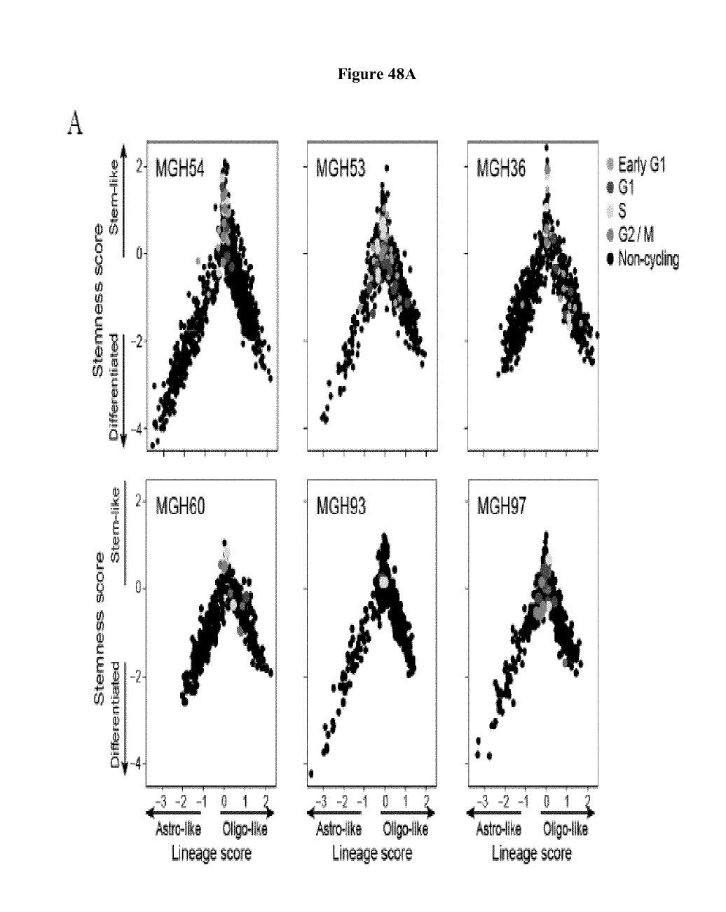

To further test the hypothesis that the stemness program is closely associated with tri-potent stem/progenitor cells. Applicants profiled by single-cell RNA-seq human neural progenitor cells (NPCs) isolated from fetal brain at 19 weeks of gestation and that can be differentiated into astrocytic, oligodendrocytic and neuronal lineages (FIG. 47a-d). While Applicants observed variation in the expression programs of these NPCs (FIG. 47e-f), unbiased PCA of the single cell NPC profiles identified a program highly similar to the PC2/3-associated program of tumor cells (FIG. 36c, FIG. 44c, Table 19: P=2*10⁻⁵, t-test). Thus, a common program is shared by subsets of our putative oligodendroglioma stem cells and normal NPCs and NSCs. Taken together, the analysis revealed three main expression patterns that recapitulate oligodendrocytic and astrocytic differentiation (PC1 high and low, respectively) and stem/progenitor programs of early neural development (PC2/3 high).

TABLE 19
Top-correlated genes (R > 0.3) for PC1 and PC2
from analysis of single cell RNA-seq of human NPCs.

| PC1 genes | PC1 correlation | PC2 genes | PC2 correlation |
|---|---|---|---|
| NEDD4L | 0.6929 | MAD2L1 | 0.8389 |
| KCNQ1OT1 | 0.6906 | ZWINT | 0.8234 |
| UGDH-AS1 | 0.6732 | MLF1IP | 0.8209 |
| ORC4 | 0.6701 | RRM2 | 0.8182 |
| IGFBPL1 | 0.6615 | CCNA2 | 0.8173 |
| SHISA9 | 0.6593 | TPX2 | 0.8106 |
| ASTN2 | 0.6347 | UBE2T | 0.7881 |
| DCX | 0.633 | KIF11 | 0.7872 |
| METTL21A | 0.6096 | MELK | 0.7859 |
| TMEM212 | 0.5971 | NCAPG | 0.7816 |
| OPHN1 | 0.5828 | MKI67 | 0.7789 |
| NRXN3 | 0.5804 | NUSAP1 | 0.7758 |
| NREP | 0.5709 | CDK1 | 0.7745 |
| ARHGEF26-AS1 | 0.557 | HMGB2 | 0.7734 |
| ODF2L | 0.551 | NCAPH | 0.7724 |
| ABCC9 | 0.5483 | KIAA0101 | 0.7716 |
| PEG10 | 0.5471 | FANCI | 0.7657 |
| SOX9 | 0.5449 | NUF2 | 0.7582 |
| SOX4 | 0.5391 | TACC3 | 0.7570 |
| TCF4 | 0.535 | PRC1 | 0.7545 |
| CHD7 | 0.5242 | CDCA5 | 0.7544 |
| UGT8 | 0.516 | FOXM1 | 0.7482 |
| DLX5 | 0.513 | CENPF | 0.7444 |
| XKR9 | 0.5036 | KIFC1 | 0.7441 |
| DLX6-AS1 | 0.4987 | TOP2A | 0.7434 |
| SOX11 | 0.4904 | KIF2C | 0.7431 |
| PDGFRA | 0.4865 | SMC2 | 0.7428 |
| DLX1 | 0.4783 | AURKB | 0.7409 |
| NPY | 0.4771 | FAM64A | 0.7375 |
| L2HGDH | 0.4728 | ASPM | 0.7325 |
| PTPRS | 0.4582 | DIAPH3 | 0.7292 |
| GLIPR1L2 | 0.4582 | UBE2C | 0.7285 |
| REXO1L1 | 0.4549 | BUB1B | 0.7279 |
| CCL5 | 0.45 | NDC80 | 0.7234 |
| CTDSP2 | 0.4476 | ASF1B | 0.7224 |
| SOX2 | 0.4444 | KIF22 | 0.7214 |
| MAB21L3 | 0.4385 | TK1 | 0.7205 |
| TP53I11 | 0.4377 | FANCD2 | 0.7182 |
| GATS | 0.437 | CASC5 | 0.7177 |
| ZFHX4 | 0.4348 | GTSE1 | 0.7144 |
| BAZ2B | 0.4323 | RRM1 | 0.7133 |
| DCLK2 | 0.4313 | RACGAP1 | 0.7126 |
| GRIA2 | 0.4286 | TYMS | 0.7095 |
| LPAL2 | 0.4274 | BIRCS | 0.7083 |
| CREBBP | 0.42 | PBK | 0.7048 |
| MARCH6 | 0.4198 | SPAG5 | 0.7004 |
| PGM5P2 | 0.4198 | KIF23 | 0.6977 |
| RERE | 0.4163 | TMPO | 0.6977 |
| SPC25 | 0.4143 | KIF15 | 0.6920 |
| GRIK3 | 0.4078 | DHFR | 0.6903 |
| CCDC88A | 0.4056 | H2AFZ | 0.6896 |
| PVRIG | 0.4038 | ANLN | 0.6871 |
| BRD3 | 0.4011 | ORC6 | 0.6857 |
| GRIA3 | 0.3996 | ARHGAP11A | 0.6809 |
| MOXD1 | 0.399 | ESCO2 | 0.6808 |
| SNTG1 | 0.3988 | KIF4A | 0.6806 |
| TAGLN3 | 0.3973 | RNASEH2A | 0.6802 |
| GSG1 | 0.3969 | RAD51AP1 | 0.6734 |
| DLX2 | 0.3946 | KIAA1524 | 0.6727 |
| ATCAY | 0.3877 | SMC4 | 0.6716 |
| NUMA1 | 0.3868 | CENPN | 0.6654 |
| LMO1 | 0.3861 | KIF18B | 0.6650 |
| POGZ | 0.3851 | VRK1 | 0.6636 |
| BPTF | 0.3849 | CCNB2 | 0.6609 |
| CHRM3 | 0.3848 | CKS1B | 0.6608 |
| RUFY3 | 0.3846 | CKAP2L | 0.6608 |
| SOX6 | 0.3833 | SHCBP1 | 0.6575 |
| RPS11 | 0.3833 | HIST1H1B | 0.6566 |
| TNFAIP8L1 | 0.3798 | SGOL1 | 0.6519 |
| FOXN3 | 0.3784 | HIST1H3B | 0.6452 |
| DAPK1 | 0.3781 | CENPM | 0.6443 |
| DLL3 | 0.373 | CCNB1 | 0.6435 |
| HERC2P4 | 0.3728 | BUB1 | 0.6434 |
| TFDP2 | 0.3724 | CENPK | 0.6433 |
| GTF2IP1 | 0.3704 | HMGN2 | 0.6427 |
| DLX6 | 0.37 | ECT2 | 0.6408 |
| IGF1R | 0.3698 | HMGB1 | 0.6399 |
| MLL3 | 0.3692 | UHRF1 | 0.6385 |
| NCAM1 | 0.368 | NCAPD2 | 0.6370 |
| CHL1 | 0.3632 | HJURP | 0.6359 |
| GNRHR2 | 0.3553 | PKMYT1 | 0.6347 |
| CLIP3 | 0.3542 | MYBL2 | 0.6333 |
| FBLIM1 | 0.3508 | CDC45 | 0.6324 |
| MATR3 | 0.3505 | CDCA2 | 0.6322 |
| CCNG2 | 0.3498 | DLGAP5 | 0.6308 |
| NEK5 | 0.3469 | TUBB | 0.6302 |
| ETV1 | 0.3454 | MCM10 | 0.6259 |
| KAT6B | 0.3448 | ATAD2 | 0.6230 |
| SRRM2 | 0.3434 | MXD3 | 0.6226 |
| FOXP1 | 0.3423 | TUBA1B | 0.6192 |
| DDX17 | 0.3408 | SGOL2 | 0.6187 |
| GOSR1 | 0.3391 | DTYMK | 0.6166 |
| GATAD2B | 0.3381 | CDC25C | 0.6162 |
| MAP4K4 | 0.3375 | TROAP | 0.6145 |
| MIAT | 0.3364 | DTL | 0.6134 |
| CD24 | 0.3327 | CDCA3 | 0.6120 |
| ZNF638 | 0.3317 | H2AFX | 0.6118 |
| HNRNPH1 | 0.3314 | LIG1 | 0.6110 |
| BRD8 | 0.3312 | TRIP13 | 0.6089 |
| MLL | 0.3285 | HAUS8 | 0.6087 |
| PCMTD1 | 0.328 | KIF20B | 0.6083 |
| AGPAT4 | 0.3251 | NCAPG2 | 0.6064 |
| YPEL1 | 0.3246 | CDKN3 | 0.6048 |
| TNIK | 0.3234 | MIS18BP1 | 0.6028 |
| PUM1 | 0.3232 | BRCA1 | 0.5958 |
| RFTN2 | 0.3231 | PLK4 | 0.5924 |
| NNAT | 0.3188 | CENPW | 0.5910 |
| MALAT1 | 0.3185 | CDC20 | 0.5845 |
| GAD1 | 0.318 | SKA3 | 0.5837 |
| ZNF37BP | 0.3172 | HIST1H4C | 0.5834 |
| IRGQ | 0.3172 | LMNB1 | 0.5828 |
| FXYD6 | 0.3165 | CDCA8 | 0.5820 |
| PRRC2B | 0.3165 | PLK1 | 0.5796 |
| FAM110B | 0.3162 | RFC3 | 0.5795 |
| YPEL3 | 0.3151 | CENPO | 0.5778 |
| ZMIZ1 | 0.3148 | DNMT1 | 0.5764 |
| CLASP1 | 0.3142 | EXO1 | 0.5741 |
| SYNE2 | 0.3134 | OIP5 | 0.5740 |
| BASP1 | 0.3134 | CHAF1A | 0.5738 |
| LYZ | 0.3133 | CENPE | 0.5713 |
| ROCK1P1 | 0.3117 | POC1A | 0.5705 |
| DPY19L2P2 | 0.3108 | DEK | 0.5663 |
| RSF1 | 0.3096 | NUCKS1 | 0.5655 |
| HIP1 | 0.3083 | MCM7 | 0.5646 |
| KANSL1 | 0.3082 | MIS18A | 0.5645 |
| ELAVL4 | 0.3079 | DEPDC1B | 0.5641 |
| TET3 | 0.3058 | CHEK1 | 0.5632 |
| ZEB2 | 0.3054 | SPC24 | 0.5623 |
| ZBTB8A | 0.3052 | GMNN | 0.5586 |
| MTSS1 | 0.3051 | PTTG1 | 0.5583 |
| TNRC6B | 0.3036 | EZH2 | 0.5565 |
| FOXO3 | 0.3032 | MCM4 | 0.5552 |
| ANKRD12 | 0.3031 | FEN1 | 0.5549 |
| MEIS3 | 0.302 | GINS1 | 0.5543 |
| JMJD1C | 0.3018 | TTK | 0.5497 |
| RICTOR | 0.3004 | CDC6 | 0.5497 |
| MEST | 0.3003 | RAD51 | 0.5495 |
| | | C19orf48 | 0.5488 |
| | | KIF20A | 0.5461 |
| | | CKAP2 | 0.5453 |
| | | CDCA4 | 0.5442 |
| | | RFC5 | 0.5441 |
| | | SKA1 | 0.5440 |
| | | CENPQ | 0.5426 |
| | | FANCA | 0.5407 |
| | | PCNA | 0.5398 |
| | | RFC4 | 0.5395 |
| | | PARP2 | 0.5390 |
| | | TMEM194A | 0.5383 |
| | | FBXO5 | 0.5360 |
| | | TIMELESS | 0.5355 |
| | | PSMC3IP | 0.5348 |
| | | HIRIP3 | 0.5316 |
| | | POLA1 | 0.5297 |
| | | RANBP1 | 0.5293 |
| | | KIF18A | 0.5291 |
| | | TCF19 | 0.5285 |
| | | USP1 | 0.5284 |
| | | LRR1 | 0.5277 |
| | | GGH | 0.5210 |
| | | HMMR | 0.5188 |
| | | CKS2 | 0.5186 |
| | | DNAJC9 | 0.5163 |
| | | SAE1 | 0.5142 |
| | | ITGB3BP | 0.5138 |
| | | TMEM106C | 0.5112 |
| | | FANCG | 0.5101 |
| | | KPNA2 | 0.5096 |
| | | NCAPD3 | 0.5078 |
| | | HELLS | 0.5071 |
| | | TMEM48 | 0.5069 |
| | | CBX5 | 0.5044 |
| | | SNRPB | 0.5011 |
| | | KNTC1 | 0.4975 |
| | | NASP | 0.4960 |
| | | MCM3 | 0.4946 |
| | | ZWILCH | 0.4933 |
| | | RPA3 | 0.4908 |
| | | CHTF18 | 0.4907 |
| | | ANP32E | 0.4903 |
| | | HIST1H3I | 0.4857 |
| | | POLA2 | 0.4854 |
| | | MZT1 | 0.4842 |
| | | MCM2 | 0.4839 |
| | | DEPDC1 | 0.4836 |
| | | DUT | 0.4835 |
| | | POLE | 0.4824 |
| | | PHIP | 0.4817 |
| | | PTMA | 0.4805 |
| | | CSE1L | 0.4786 |
| | | DSCC1 | 0.4780 |
| | | CDC7 | 0.4764 |
| | | HMGB3 | 0.4756 |
| | | TUBB4B | 0.4748 |
| | | STMN1 | 0.4747 |
| | | RPA2 | 0.4739 |
| | | RCC1 | 0.4726 |
| | | CENPH | 0.4719 |
| | | GINS2 | 0.4712 |
| | | EXOSC9 | 0.4710 |
| | | NCAPH2 | 0.4708 |
| | | NUDT15 | 0.4697 |
| | | SPC25 | 0.4674 |
| | | HNRNPA2B1 | 0.4674 |
| | | MND1 | 0.4643 |
| | | DSN1 | 0.4631 |
| | | MASTL | 0.4607 |
| | | RAD21 | 0.4604 |
| | | PHGDH | 0.4603 |
| | | ZNF331 | 0.4594 |
| | | RANGAP1 | 0.4588 |
| | | SAPCD2 | 0.4582 |
| | | PARPBP | 0.4579 |
| | | ANP32B | 0.4562 |
| | | SMC1A | 0.4554 |
| | | NEK2 | 0.4527 |
| | | BARD1 | 0.4526 |
| | | NIF3L1 | 0.4520 |
| | | PRR11 | 0.4506 |
| | | HNRNPD | 0.4500 |
| | | MCM5 | 0.4480 |
| | | SMC3 | 0.4479 |
| | | FAM111A | 0.4473 |
| | | POLD1 | 0.4460 |
| | | CDK2 | 0.4458 |
| | | FUS | 0.4426 |
| | | PHF19 | 0.4399 |
| | | ARHGAP33 | 0.4345 |
| | | NUP205 | 0.4344 |
| | | CDC25B | 0.4335 |
| | | PA2G4 | 0.4323 |
| | | NUDT1 | 0.4311 |
| | | CHEK2 | 0.4307 |
| | | WDR34 | 0.4305 |
| | | H2AFY | 0.4271 |
| | | HAUS1 | 0.4239 |
| | | BUB3 | 0.4236 |
| | | CHAF1B | 0.4206 |
| | | PRIM2 | 0.4190 |
| | | CCDC34 | 0.4176 |
| | | POLE2 | 0.4175 |
| | | PRPS2 | 0.4174 |
| | | RFWD3 | 0.4171 |
| | | UBR7 | 0.4155 |
| | | CCNE2 | 0.4145 |
| | | RAN | 0.4144 |
| | | DDX11 | 0.4142 |
| | | NUP50 | 0.4131 |
| | | CACYBP | 0.4128 |
| | | HNRNPAB | 0.4123 |
| | | DBF4 | 0.4120 |
| | | TMSB15A | 0.4114 |
| | | AURKA | 0.4106 |
| | | MAD2L2 | 0.4095 |
| | | GINS3 | 0.4095 |
| | | ASRGL1 | 0.4086 |
| | | PPIF | 0.4084 |
| | | CKAP5 | 0.4060 |
| | | UBE2S | 0.4053 |
| | | LMNB2 | 0.4040 |
| | | POLD3 | 0.4039 |
| | | TEX30 | 0.4002 |
| | | SUV39H1 | 0.3999 |
| | | CCP110 | 0.3997 |
| | | WHSC1 | 0.3988 |
| | | MCM6 | 0.3986 |
| | | ACYP1 | 0.3983 |
| | | GNG4 | 0.3957 |
| | | PRIM1 | 0.3933 |
| | | NSMCE4A | 0.3920 |
| | | EXOSC8 | 0.3916 |
| | | COMMD4 | 0.3910 |
| | | SNRPD1 | 0.3887 |
| | | HAT1 | 0.3885 |
| | | H2AFV | 0.3870 |
| | | CMC2 | 0.3868 |
| | | SSRP1 | 0.3858 |
| | | HIST1H1E | 0.3852 |
| | | RBMX | 0.3844 |
| | | LBR | 0.3842 |
| | | RPL39L | 0.3818 |
| | | EMP2 | 0.3818 |
| | | CENPL | 0.3813 |
| | | CEP78 | 0.3809 |
| | | TRAIP | 0.3807 |
| | | COPS3 | 0.3781 |
| | | LSM4 | 0.3779 |
| | | RBBP8 | 0.3774 |
| | | HIST1H1C | 0.3743 |
| | | RPA1 | 0.3733 |
| | | RAD1 | 0.3714 |
| | | NUP210 | 0.3712 |
| | | HSPB11 | 0.3701 |
| | | RFC2 | 0.3684 |
| | | ACTL6A | 0.3671 |
| | | SRRT | 0.3663 |
| | | NUP107 | 0.3655 |
| | | GPN3 | 0.3614 |
| | | LSM3 | 0.3606 |
| | | SUV39H2 | 0.3602 |
| | | POLR2D | 0.3597 |
| | | HAUS5 | 0.3594 |
| | | WDR76 | 0.3588 |
| | | LSM5 | 0.3575 |
| | | NXT1 | 0.3563 |
| | | TUBG1 | 0.3557 |
| | | C16orf59 | 0.3554 |
| | | REEP4 | 0.3539 |
| | | BTG3 | 0.3538 |
| | | RNASEH2B | 0.3538 |
| | | TUBB6 | 0.3534 |
| | | PPIA | 0.3524 |
| | | RBL1 | 0.3522 |
| | | ARL6IP6 | 0.3504 |
| | | COX17 | 0.3501 |
| | | SYNE2 | 0.3500 |
| | | GUSB | 0.3499 |
| | | MSH5 | 0.3479 |
| | | CRNDE | 0.3472 |
| | | DDX39A | 0.3467 |
| | | SUPT16H | 0.3467 |
| | | HNRNPUL1 | 0.3455 |
| | | POLE3 | 0.3454 |
| | | HAUS4 | 0.3449 |
| | | IDH2 | 0.3448 |
| | | H1FX | 0.3439 |
| | | DCP2 | 0.3427 |
| | | NUP188 | 0.3417 |
| | | MPHOSPH9 | 0.3415 |
| | | PPIG | 0.3407 |
| | | MAGOHB | 0.3400 |
| | | RIF1 | 0.3393 |
| | | MLH1 | 0.3386 |
| | | MSH2 | 0.3367 |
| | | SNRNP40 | 0.3363 |
| | | HADH | 0.3346 |
| | | GABPB1 | 0.3341 |
| | | NUDC | 0.3332 |
| | | PHTF2 | 0.3328 |
| | | NUP85 | 0.3325 |
| | | NUP35 | 0.3316 |
| | | SKP2 | 0.3310 |
| | | THOC3 | 0.3292 |
| | | ANAPC11 | 0.3283 |
| | | TFAM | 0.3283 |
| | | AKR1B1 | 0.3281 |
| | | ILF2 | 0.3276 |
| | | TMEM237 | 0.3268 |
| | | RAD54B | 0.3258 |
| | | SMPD4 | 0.3258 |
| | | HMGN1 | 0.3255 |
| | | CBX3 | 0.3253 |
| | | TPRKB | 0.3250 |
| | | GGCT | 0.3249 |
| | | FBL | 0.3249 |
| | | RFC1 | 0.3247 |
| | | CCT5 | 0.3231 |
| | | PRKDC | 0.3222 |
| | | CDK5RAP2 | 0.3221 |
| | | SRSF2 | 0.3204 |
| | | CEP112 | 0.3191 |
| | | LDHA | 0.3189 |
| | | SRSF3 | 0.3183 |
| | | HSP90AA1 | 0.3179 |
| | | SRSF7 | 0.3175 |
| | | HAUS6 | 0.3150 |
| | | CCHCR1 | 0.3143 |
| | | CEP57 | 0.3135 |
| | | HMGA1 | 0.3129 |
| | | UCHL5 | 0.3122 |
| | | C1orf174 | 0.3120 |
| | | CTPS1 | 0.3120 |
| | | ACOT7 | 0.3119 |
| | | SNHG1 | 0.3119 |
| | | PSMC3 | 0.3116 |
| | | ZNF93 | 0.3106 |
| | | 10/sep | 0.3100 |
| | | PCM1 | 0.3091 |
| | | SFPQ | 0.3089 |
| | | RMI1 | 0.3084 |
| | | NUP37 | 0.3057 |
| | | DCK | 0.3056 |
| | | AHI1 | 0.3052 |
| | | SVIP | 0.3051 |
| | | CHCHD2 | 0.3049 |
| | | ZNF714 | 0.3049 |
| | | XRCC5 | 0.3048 |
| | | NFATC2IP | 0.3040 |
| | | SLC25A5 | 0.3036 |
| | | WRAP53 | 0.3034 |
| | | PSIP1 | 0.3029 |
| | | MRPS6 | 0.3021 |
| | | NT5DC2 | 0.3015 |
| | | NOP58 | 0.3003 |

To precisely assign a cellular state to each individual tumor cell, Applicants defined an OC vs. AC lineage score and a sternness vs. differentiation score (Methods). Plotting these two scores across the cells of all three tumors together revealed a striking similarity to normal cellular hierarchies (FIG. 36d), with a transition from a stem/progenitor program branching into differentiation along two glial lineages. Importantly, the same architecture was observed in each of the six tumors (FIG. 36e, FIG. 47). Statistical analysis of the variation in lineage score compared to expected technical noise suggests that the transition involves intermediate states for each lineage (FIG. 48), but the exact number of states and whether they are discrete or form a more continuous trajectory is difficult to determine due to technical limitations associated with noise in single cell RNA-seq data (Note 2).

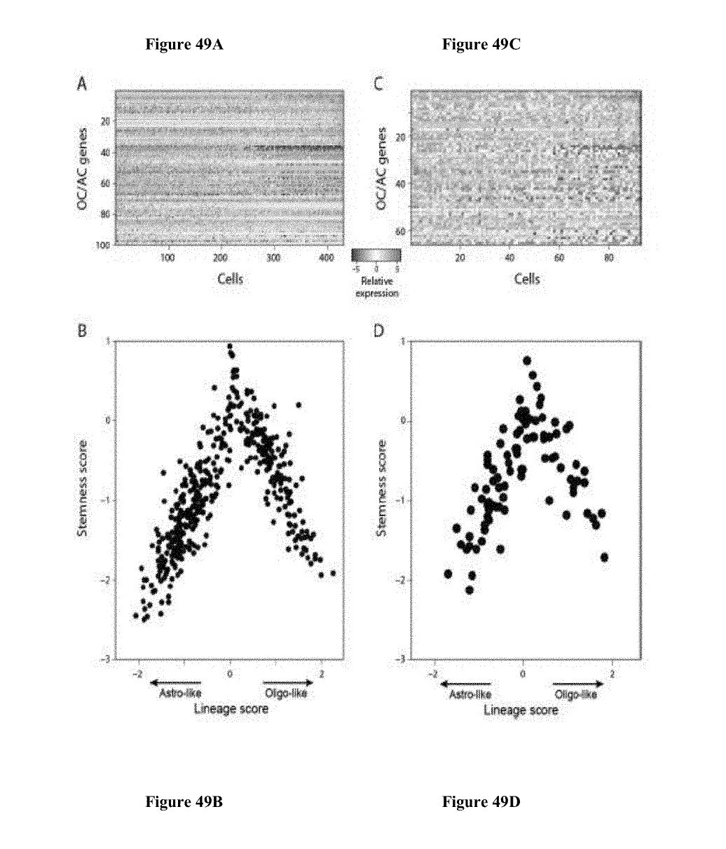

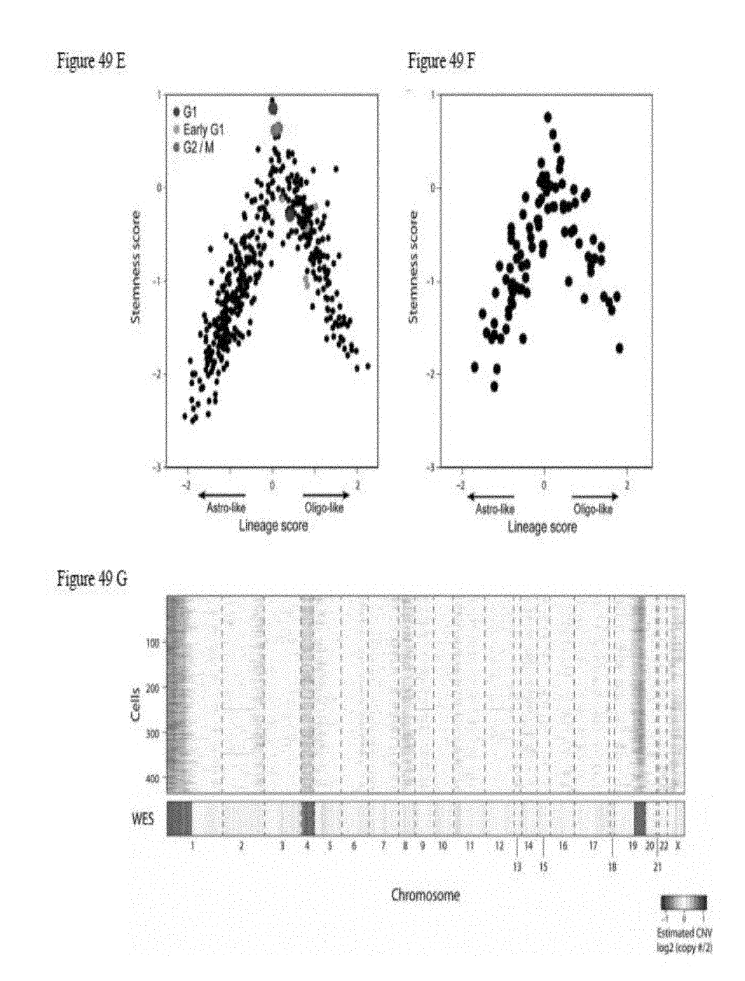

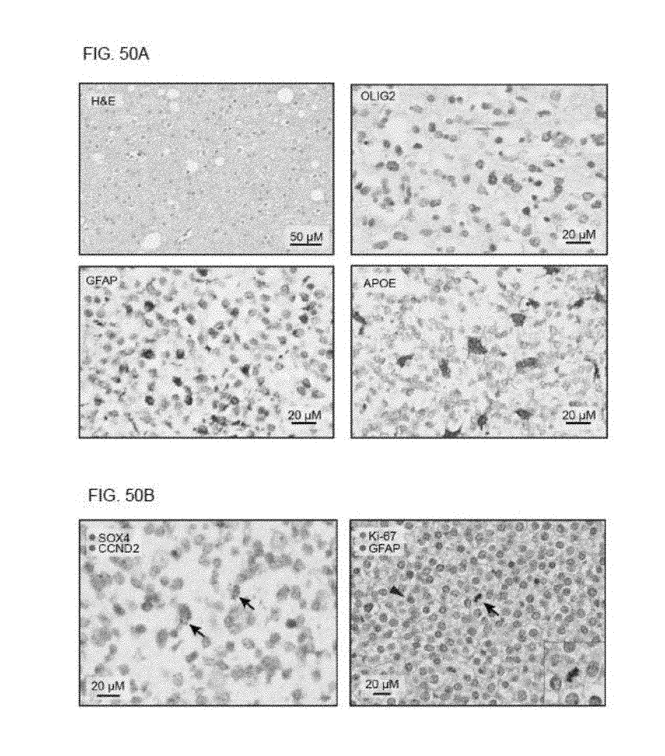

Applicants validated the generality of these findings in two ways. First, Applicants observed the same architecture when Applicants independently profiled one of the tumors (MGH60) with a different method for single cell RNA-seq (Methods; FIG. 49). Second, Applicants confirmed these patterns in tumors by both RNA in situ hybridization and immunohistochemistry with markers of AC (GFAP and APOE), OC (OLIG2, OMG) and stem/progenitor cells (SOX4, CCND2) performed in each of the original 6 tumors and in a validation cohort often additional tumors (FIG. 36f,g, FIG. 50 and Table 20).

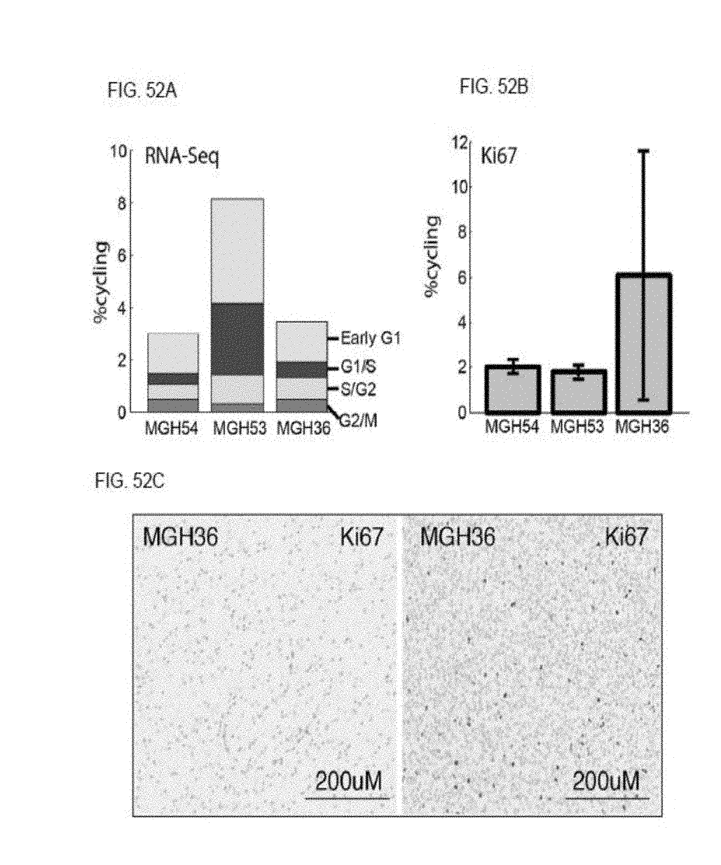

This architecture suggests a developmental hierarchy in which tumor stem/progenitor cells give rise to differentiated progeny. To assess how patterns of tumor proliferation and self-renewal may relate to the developmental hierarchy. Applicants next scored each cell for the expression of consensus gene sets for the G1/S phases and the G2/M phases, which Applicants defined based on consistent association with those phases across multiple datasets (Methods) (16, 124) Applicants found that only a small proportion of cells in each tumor (1.5-8%) are proliferating (FIG. 37a, FIG. 51-52). The fraction of proliferating cells Applicants identified by expression program is within the expected range for oligodendrogliomas and comparable to the percentage of cycling cells identified by Ki-67 staining in these tumors, with the caveat that proliferation can vary substantially between different regions of the same tumor (FIG. 52). Applicants further distinguished cycling cells by their G1/S and G2/M scores, to identify four distinct cell cycle phases (FIG. 37a).

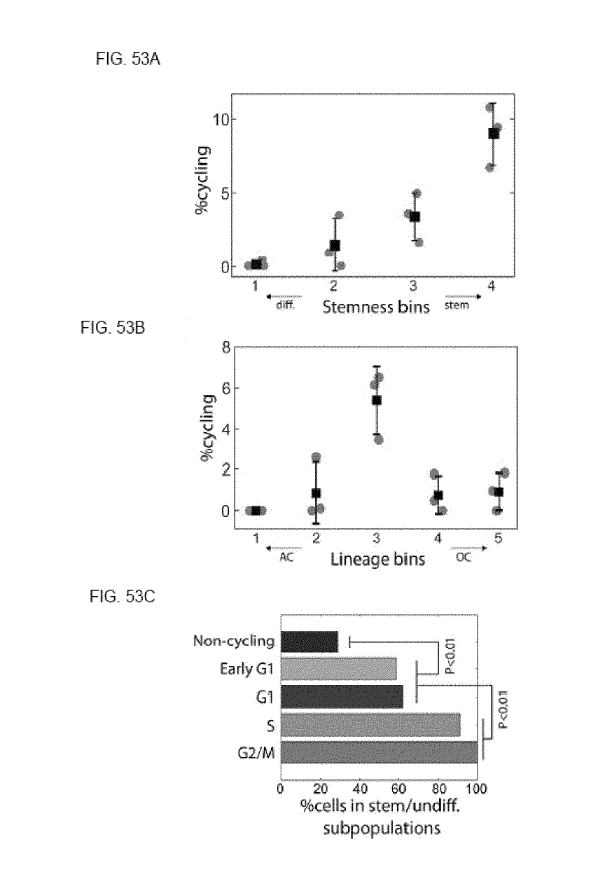

Strikingly, almost all cycling cancer cells were confined to the stem/progenitor and undifferentiated compartment of the tumor (FIG. 37b,c, FIG. 53a,b), suggesting that this represents the compartment responsible for the growth of oligodendrogliomas in humans. Several lines of evidence support the finding that stem/progenitor and undifferentiated cells account for tumor proliferation. First, Applicants validated the co-expression of a stem/progenitor marker (SOX4) and the cell proliferation marker (Ki67) in tissue staining across 14 patients, as well as a negative correlation for cycling and glial differentiation markers (FIG. 37d and FIG. 50 and Table 20). Second, there is a strong correlation between our cell-cycle signature and our stem/progenitor signature across 69 bulk oligodendroglioma samples in the TCGA dataset (FIG. 37e) (112). Finally, the enrichment of cell cycle among stem/progenitor and undifferentiated cells was even more striking for cells inferred to be in G2/M phases compared to those in the G1 phase (FIG. 53c), possibly reflecting the short G1 phase observed in tissue and embryonic stem cells (113).

TABLE 20
Fraction of cells in each subpopulation as estimated by single cell RNA-seq (top) and tissue staining (bottom)

| | | | | | Cycling stem- | Cycling stem- | Cycling OC- | Cycling AC- | | | |
|---|---|---|---|---|---|---|---|---|---|---|---|
| | OC- | AC- | Stem- | | like (with | like + undif. | like (with | like (with | OC + | OC + | AC + |
| | like | like | like | Undif. | early G1) | (with early G1) | early G1) | early G1) | AC | stem | stem |
| MGH36 | 34.21% | 49.20% | 10.04% | 6.55% | 0.72% (1.01%) | 1.15% (1.44%) | 0.43% (101%) | 0% (0.14%) | 0.15% | 4.22% | 1.60% |
| MGH53 | 33.64% | 17.33% | 14.35% | 29.69% | 0.55% (1.65%) | 2.62% (4.96%) | 0.14% (0.14%) | 0% (0.14%) | 0.14% | 0.43% | 0.99% |
| MGH54 | 44.57% | 23.10% | 16.90% | 15.43% | 0.77% (1.53%) | 1.28% (2.56%) | 0% (0%) | 0% (0.09%) | 0.17% | 1.29% | 0.78% |
| MGH60 | 34.66% | 50.82% | 4.22% | 10.30% | 0.47% (0.93%) | 0.7% (2.09%) | 0.23% (0.7%) | 0% (0.7%) | 0.00% | 3.28% | 0.23% |
| Average | 38.02% | 35.11% | 11.38% | 15.49% | 0.63% (1.28%) | 1.44% (2.76%) | 0.2% (0.46%) | 0% (0.27%) | 0.12% | 2.31% | 0.90% |

| | OMG | APOE | SOX4 | SOX4 + Ki67 | CCND2 + SOX4 | CCND2 + OMG | CCND2 + APOE |
|---|---|---|---|---|---|---|---|
| MGH36 | 31.00% | 41.00% | 8.00% | 2.10% | 1.90% | 0.20% | 0% |
| MGH53 | 30.00% | 15.00% | 12.00% | 1.30% | 1.00% | 0% | 0% |
| MGH54 | 37.00% | 25.00% | 9.00% | 0.90% | 1.10% | 0.20% | 0% |
| Oligo 1 | 28.00% | 26.00% | 7.00% | 0.90% | 1.00% | 0% | 0% |
| Oligo 2 | 31.00% | 17.00% | 2.00% | 0.90% | 1.00% | 0% | 0.10% |
| Oligo 3 | 43.00% | 19.00% | 6.00% | 1.60% | 1.30% | 0% | 0% |
| Oligo 4 | 45.00% | 11.00% | 8.00% | 1.90% | 2.00% | 0.30% | 0.10% |
| Oligo 5 | 24.00% | 30.00% | 3.00% | 0.90% | 1.00% | 0% | 0% |
| Oligo 6 | 12.00% | 47.00% | 5.00% | 0.30% | 0.90% | 0% | 0% |
| Oligo 7 | 22.00% | 35.00% | 4.00% | 3.00% | 4.00% | 0.50% | 0.50% |
| Oligo 8 | 25.00% | 37.00% | 2.00% | 1.30% | 1.50% | 0% | 0.20% |
| Oligo 9 | 27.00% | 33.00% | 7.00% | 0.50% | 1.00% | 0.10% | 0% |
| Oligo 10 | 36.00% | 29.00% | 9.00% | 0.70% | 0.90% | 0% | 0% |
| Average | 30.00% | 28.50% | 6.30% | 1.25% | 1.43% | 0.10% | 0.07% |

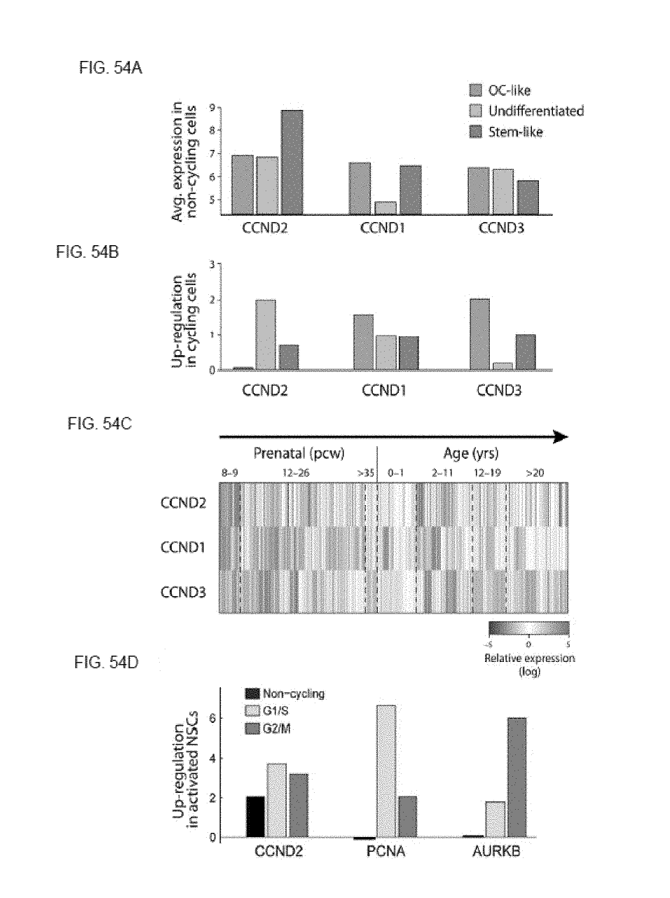

Although cycling cells were highly enriched among stem/progenitors, the frequency of cycling cells was low (˜10%) even among stem/progenitors. Because cycling cells are a minority even among stem/progenitor cells, the PC2/3 stem/progenitor program did not include a signature for cell cycle. The notable exception is CCND2 (FIG. 36a), a gene which plays a major role in controlling the cell cycle and was previously associated with self-renewal of glioma CSC (114). Interestingly, CCND2 was highly expressed both in cycling cells as well in non-cycling stem/progenitor cells (FIG. 54a,b), consistent with previous work that implicated it in priming cells to enter the cell cycle (113). Stem/progenitor tumor cells preferentially express CCND2, whereas differentiated tumor cells express CCND1 and CCND3, mirroring the high expression of CCND2 in early neurodevelopment, which is later replaced by CCND1 and CCND3 (FIG. 54c). CCND2 was also upregulated in activated mouse NSCs prior to entering the cell cycle (FIG. 54d). Taken together, these results indicate a role of CCND2 in both normal and malignant neural stem cell programs.

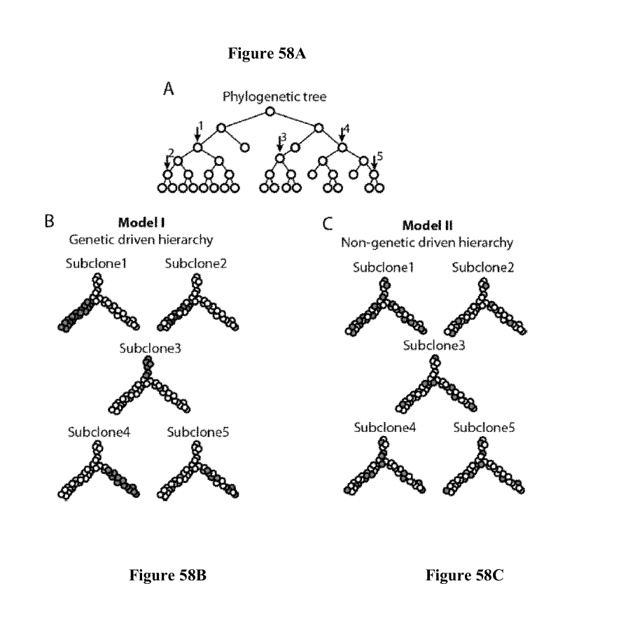

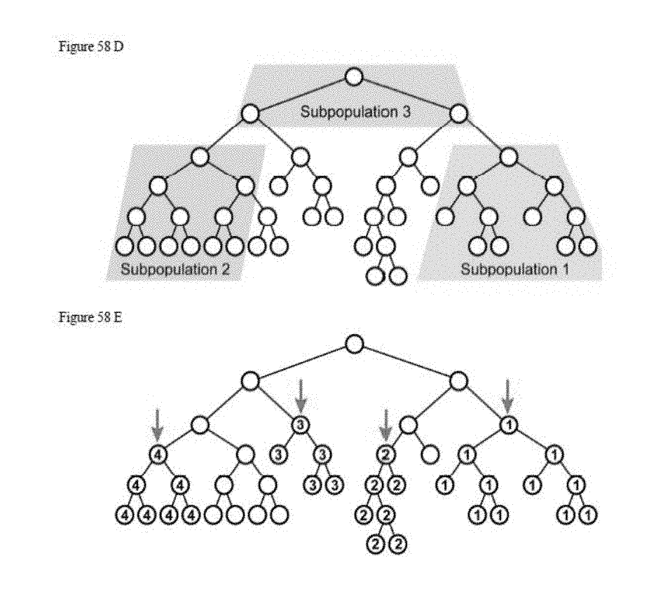

Finally, Applicants explored the role of genetic events in shaping the cellular identity, devising two approaches to obtain genetic information from single cell RNA-seq and classify cells into tumor subclones. In the first approach, Applicants used the CNV inference (FIG. 35b,c) of each cell to relate its genetic state with its transcriptional profile. In this approach, Applicants can ascertain the CNV features for every cell, but the number of genetic features is small (few CNVs). In the second approach, Applicants identified subclonal point mutations from bulk DNA whole-exome sequencing, using the ABSOLUTE method (35), and then searched for these mutations in the RNA-seq reads of individual cells (Methods). This approach assesses a larger number of mutations, but its sensitivity is limited by RNA-seq coverage, heterozygosity and allele-specific expression, such that Applicants could only ascertain (observe) mutations in a small fraction of cells compared to the expected subclonal fraction (Methods). Applicants performed whole-exome sequencing from bulk tumors and matched blood, identified tumor-specific single-point mutations (Table 21) and mapped them to our single profiled cells based on RNA-seq reads that harbored these exact mutations (FIG. 38c). However, the confidence of the ascertained mutations is illustrated by a low estimated false positive rate (<1%) (Methods) and by validation of a subset of mutations by qPCR (below) and targeted sequencing (Methods). The genetic information obtained with these two approaches is partial and is not sufficient to reconstruct a full phylogenetic tree. However, Applicants reasoned that it is sufficient to test if each subclonal genetic feature is restricted to a certain developmental state or if alternatively, according to the model of non-genetically-driven hierarchy, subclones span distinct developmental states (FIG. 58).

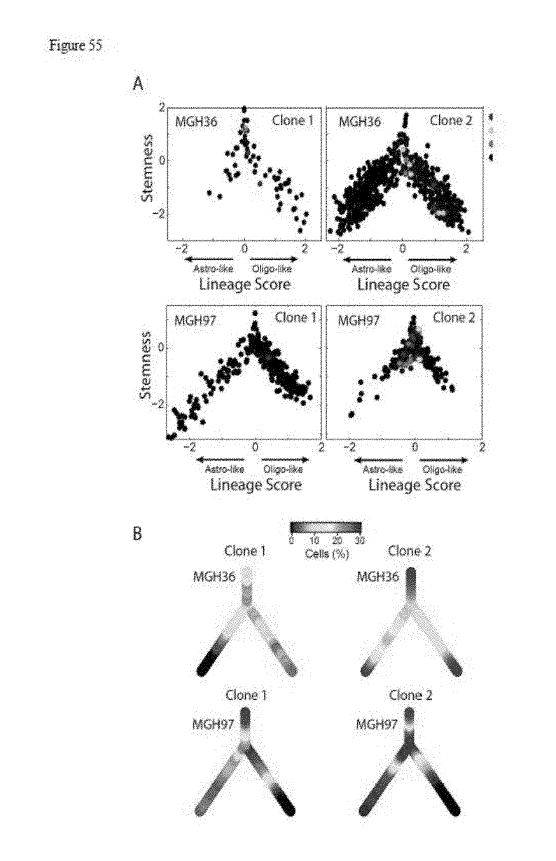

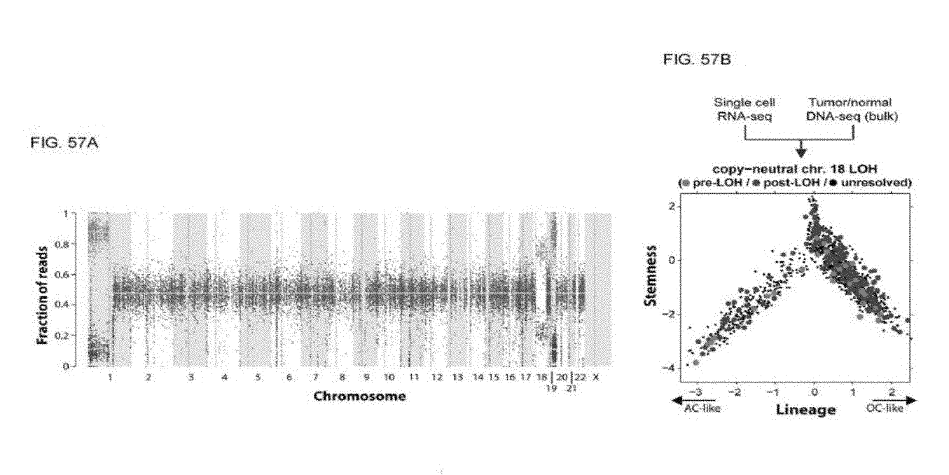

Applicants observed the same 3 sub-population architecture within distinct CNV sub-clones in MGH36 and in MGH97 (FIG. 35c), with cycling stem/progenitor cells and two lineages of differentiated non-cycling cells (FIG. 38a,b, FIG. 55). This suggests that distinct CNV profiles do not dictate a specific cellular state, and rather that developmental programs are over-imposed over CNV clones. Similarly, examining the distribution of transcriptional states for cells that harbor subclonal point mutations, Applicants found that 23 subclonal point mutations (FIG. 38c,d and FIG. 56) and a subclonal loss-of-heterozygosity event (FIG. 57) are not significantly restricted to particular developmental states and often span all three states. In particular, these include multiple cases with low subclonal fraction (<12% based on ABSOLUTE) that nevertheless span all three compartments in the transcriptional hierarchy (e.g., point mutations in ZEB2, EEF1B2, FTH1, FRG1B, and CNV clone 1 in MGH36). Regardless of whether a mutation has low fraction because it arose early (and did not rise in frequency) or late (and is thus a minor deep branch), the fact that it spans all compartments strongly argues against a genetic explanation.

Thus, our approach, applied across CNVs and multiple point mutations provides many examples of distinct genetic subclones that span the developmental hierarchy. This indicates that oligodendroglioma's developmental hierarchy is largely maintained during genetic evolution. The presence of a similar hierarchy in each of the tumors examined and across multiple subclones within each tumor, together with the lack of shared subclonal mutations across these oligodendrogliomas, strongly argues that the hierarchy is not driven by genetics.

TABLE 21
Mutations identified by DNA whole exome sequencing of tumor tissue and matched blood, their ABSOLUTE-estimated clonal fraction

| | | | | | | | | |
|---|---|---|---|---|---|---|---|---|
| | | | cancer cell | | | | | |
| | | | fraction | Variant_ | Reference_ | Alternative_ | | Protein_ |
| Hugo_Symbol | Chromosome | position | (ABSOLUTE) | Classification | Allele | Allele | cDNA_Change | Change |

MGH53

| | | | | | | | | |
|---|---|---|---|---|---|---|---|---|
| DDX11L1 | 1 | 15906 | 0.28 | RNA | A | G | | |
| DDX11L1 | 1 | 15922 | 0.21 | RNA | A | G | | |
| PLCH2 | 1 | 2435349 | 1 | Intron | A | C | | |
| PLCH2 | 1 | 2435352 | 0.89 | Intron | T | C | | |
| PLCH2 | 1 | 2435357 | 1 | Intron | A | C | | |
| NBPF1 | 1 | 16892724 | 0.04 | Intron | A | T | | |
| Unknown | 1 | 16974745 | 0.08 | IGR | G | A | | |
| ZNF362 | 1 | 33747370 | 0.96 | Missense_Mutation | A | G | c.866A > G | p.D289G |
| OSBPL9 | 1 | 52226257 | 0.64 | Intron | T | G | | |
| IGSF3 | 1 | 117158772 | 0.13 | Silent | C | T | c.351G > A | p.E117E |
| LCE1A | 1 | 152799987 | 0.5 | Silent | T | C | c.39T > C | p.P13P |
| PMVK | 1 | 154897570 | 1 | 3′UTR | T | C | | |
| THBS3 | 1 | 155167452 | 0.6 | Splice_Site | T | G | | |
| KIAA0907 | 1 | 155887387 | 0.76 | Missense_Mutation | T | G | c.1343A > C | p.Q448P |
| KIAA0907 | 1 | 155887393 | 0.58 | Missense_Mutation | T | G | c.1337A > C | p.Q446P |
| SH2D2A | 1 | 156777070 | 0.61 | Missense_Mutation | T | G | c.1070A > C | p.Q357P |
| SH2D2A | 1 | 156777073 | 0.79 | Missense_Mutation | T | G | c.1067A > C | p.H356P |
| DARS2 | 1 | 173795839 | 0.2 | Missense_Mutation | G | T | c.142G > T | p.V48F |
| CR1 | 1 | 207787753 | 0.1 | Nonsense_Mutation | C | T | c.6580C > T | p.R2194* |
| LYST | 1 | 235938295 | 0.11 | Missense_Mutation | T | G | c.5552A > C | p.E1851A |
| FMN2 | 1 | 240371436 | 0.35 | Silent | T | C | c.3324T > C | p.P1108P |
| CEP170 | 1 | 243319558 | 0.25 | Silent | G | T | c.3876C > A | p.I1292I |
| CEP170 | 1 | 243333027 | 0.12 | Silent | A | G | c.1746T > C | p.R582R |
| KIF26B | 1 | 245765965 | 0.11 | Missense_Mutation | G | T | c.1437G > T | p.K479N |
| C2orf71 | 2 | 29293879 | 0.31 | Silent | A | G | c.3249T > C | p.P1083P |
| ALK | 2 | 29455195 | 0.55 | Silent | C | A | c.2607G > T | p.G869G |
| EIF2AK2 | 2 | 37374837 | 0.29 | Missense_Mutation | T | G | c.113A > C | p.D38A |
| CTNNA2 | 2 | 80136918 | 0.59 | Missense_Mutation | A | C | c.1051A > C | p.N351H |
| IL1RL2 | 2 | 102835512 | 0.21 | Missense_Mutation | A | C | c.824A > C | p.D275A |
| RGPD3 | 2 | 107049681 | 0.04 | Missense_Mutation | T | C | c.2266A > G | p.N756D |
| FOXD4L1 | 2 | 114256759 | 0.21 | 5′UTR | A | G | | |
| KIF5C | 2 | 149633151 | 1 | 5′UTR | A | C | | |
| KIF5C | 2 | 149633155 | 0.98 | 5′UTR | A | C | | |
| KIF5C | 2 | 149633161 | 0.68 | 5′UTR | G | C | | |
| RAPH1 | 2 | 204322299 | 0.09 | Missense_Mutation | T | C | c.1112A > G | p.K371R |
| ADAM23 | 2 | 207452868 | 0.09 | Silent | C | A | c.1842C > A | p.I614I |
| CPO | 2 | 207833951 | 0.34 | Missense_Mutation | T | G | c.916T > G | p.S306A |
| IDH1 | 2 | 209113112 | 0.95 | Missense_Mutation | C | T | c.395G > A | p.R132H |
| IRS1 | 2 | 227660628 | 0.14 | Missense_Mutation | T | G | c.2827A > C | p.K943Q |
| UBE2F-SCLY | 2 | 238965872 | 0.28 | 3′UTR | T | A | | |
| TPRXL | 3 | 14106174 | 0.28 | Silent | T | C | c.498T > C | p.S166S |
| NR2C2 | 3 | 15084335 | 0.77 | Intron | TT | GG | | |
| NGLY1 | 3 | 25770654 | 0.42 | Silent | T | G | c.1581A > C | p.I527I |
| PLXNB1 | 3 | 48461609 | 0.5 | Missense_Mutation | T | G | c.2086A > C | p.T696P |
| PLXNB1 | 3 | 48461613 | 0.49 | Silent | T | G | c.2082A > C | p.P694P |
| BTLA | 3 | 112198364 | 0.14 | Missense_Mutation | C | T | c.341G > A | p.R114H |
| PIK3CB | 3 | 138433351 | 0.77 | Missense_Mutation | T | G | c.1261A > C | p.N421H |
| CLRN1 | 3 | 150645448 | 0.15 | 3′UTR | T | C | | |
| P2RY12 | 3 | 151055868 | 0.34 | Nonsense_Mutation | G | A | c.766C > T | p.R256* |
| EGFEM1P | 3 | 168530083 | 0.81 | RNA | A | T | | |
| MUC4 | 3 | 195507144 | 0.07 | Silent | C | T | c.11307G > A | p.V3769V |
| MUC4 | 3 | 195513285 | 0.05 | Silent | G | T | c.5166C > A | p.S1722S |
| MFI2 | 3 | 196736499 | 0.21 | Silent | G | A | c.1515C > T | p.D505D |
| ATP5I | 4 | 667819 | 0.35 | Intron | A | G | | |
| CLOCK | 4 | 56304585 | 0.2 | Missense_Mutation | G | A | c.2225C > T | p.A742V |
| PDCL2 | 4 | 56435894 | 0.43 | Missense_Mutation | T | G | c.353A > C | p.Y118S |
| GYPE | 4 | 144797983 | 0.91 | Silent | C | T | c.162G > A | p.A54A |
| PDE4D | 5 | 58295396 | 0.18 | Intron | G | A | | |
| KIF2A | 5 | 61602215 | 1 | 5′UTR | T | C | | |
| NBPF22P | 5 | 85589141 | 0.07 | RNA | T | G | | |
| SYCP2L | 6 | 10942975 | 0.21 | Missense_Mutation | C | A | c.1950C > A | p.D650E |
| ACOT13 | 6 | 24701717 | 0.32 | Missense_Mutation | T | G | c.297T > G | D.D99E |
| BTN2A3P | 6 | 26422353 | 0.13 | RNA | C | T | | |
| ZNF165 | 6 | 28053590 | 0.34 | Missense_Mutation | A | C | c.332A > C | p.E111A |
| Unknown | 6 | 29856906 | 0.17 | IGR | G | A | | |
| NRM | 6 | 30658769 | 0.46 | 5′UTR | T | G | | |
| BAG6 | 6 | 31610160 | 0.78 | Silent | T | G | c.1974A > C | p.P658P |
| GPR116 | 6 | 46856205 | 0.12 | Silent | A | G | c.195T > C | p.V65V |
| PTP4A1 | 6 | 64289971 | 0.25 | Silent | T | G | c.414T > G | p.R138R |
| ZNF292 | 6 | 87965630 | 0.38 | Missense_Mutation | T | G | c.2283T > G | p.F761L |
| ORC3 | 6 | 88318940 | 1 | Missense_Mutation | A | C | c.706A > C | p.I236L |
| CDC40 | 6 | 110534309 | 0.86 | Missense_Mutation | G | T | c.888G > T | p.L296F |
| LAMA2 | 6 | 129371133 | 0.03 | Silent | A | G | c.183A > G | p.K61K |
| VNN1 | 6 | 133014444 | 1 | Missense_Mutation | A | C | c.545T > G | p.F182C |
| MAP7 | 6 | 136699003 | 0.34 | Missense_Mutation | C | T | c.641G > A | p.R214H |
| UNC93A | 6 | 167728954 | 0.16 | 3′UTR | C | T | | |
| FAM120B | 6 | 170627052 | 0.44 | Missense_Mutation | T | G | c.574T > G | p.S192A |
| PHF14 | 7 | 11013807 | 1 | 5′UTR | G | A | | |
| H2AFV | 7 | 44874056 | 0.13 | 3′UTR | A | C | | |
| ABCA13 | 7 | 48232645 | 0.18 | Silent | C | T | c.159C > T | p.D53D |
| TMEM248 | 7 | 66413644 | 0.26 | Missense_Mutation | A | C | c.559A > C | p.T187P |
| POM121 | 7 | 72398976 | 0.06 | Missense_Mutation | A | G | c.1076A > G | p.N359S |
| POM121 | 7 | 72413896 | 0.06 | Missense_Mutation | A | G | c.3364A > G | p.T1122A |
| COL1A2 | 7 | 94052281 | 0.62 | Missense_Mutation | C | T | c.2416C > T | p.P806S |
| LRRC17 | 7 | 102585014 | 0.19 | Missense_Mutation | C | G | c.1286C > G | p.T429S |
| LRRN3 | 7 | 110763972 | 0.16 | Missense_Mutation | A | C | c.1144A > C | p.N382H |
| KMT2C | 7 | 151970855 | 0.02 | Missense_Mutation | G | C | c.947C > G | p.T316S |
| Unknown | 8 | 12517307 | 0.14 | IGR | C | T | | |
| PDLIM2 | 8 | 22447026 | 0.87 | Intron | A | C | | |
| LRRCC1 | 8 | 86019547 | 0.2 | Missense_Mutation | C | T | c.17C > T | p.A6V |
| TG | 8 | 134147138 | 0.83 | 3′UTR | G | A | | |
| COL22A1 | 8 | 139824118 | 0.58 | Missense_Mutation | T | G | c.1373A > C | p.Q458P |
| COL22A1 | 8 | 139824129 | 1 | Silent | T | G | c.1362A > C | p.P454P |
| TSTA3 | 8 | 144697039 | 0.54 | Missense_Mutation | T | G | c.308A > C | p.E103A |
| CPSF1 | 8 | 145620768 | 0.57 | Splice_Site | T | G | | |
| KIFC2 | 8 | 145694024 | 0.78 | Missense_Mutation | C | A | c.994C > A | p.Q332K |
| SMU1 | 9 | 33068870 | 0.08 | Silent | G | A | c.453C > T | p.G151G |
| FAM20SB | 9 | 34835480 | 0.06 | RNA | C | T | | |
| GLIPR2 | 9 | 36147796 | 0.25 | Missense_Mutation | T | G | c.27T > G | p.F9L |
| MIR4477B | 9 | 68414704 | 0.41 | RNA | A | C | | |
| MIR4477B | 9 | 68414853 | 0.48 | RNA | C | T | | |
| Unknown | 9 | 69067873 | 0.5 | IGR | A | C | | |
| Unknown | 9 | 69067929 | 0.58 | IGR | G | A | | |
| CCDC180 | 9 | 100105896 | 0.52 | Intron | C | A | | |
| CDK5RAP2 | 9 | 123151373 | 0.29 | 3′UTR | A | G | | |
| LCN1 | 9 | 138413373 | 0.11 | Silent | T | C | c.30T > C | p.L10L |
| TSPAN15 | 10 | 71267418 | 0.23 | 3′UTR | T | G | | |
| BTBD10 | 11 | 13435092 | 0.36 | Missense_Mutation | T | G | c.793A > C | p.K265Q |
| OR4C6 | 11 | 55433000 | 0.9 | Missense_Mutation | C | T | c.358C > T | p.R120C |
| FOSL1 | 11 | 65664326 | 0.95 | Missense_Mutation | C | T | c.251G > A | p.R84Q |
| UNC93B1 | 11 | 67759316 | 0.13 | Missense_Mutation | C | T | c.1492G > A | p.V498M |
| GRAMD1B | 11 | 123431287 | 0.58 | Intron | A | C | | |
| TIRAP | 11 | 126162750 | 0.15 | Missense_Mutation | C | T | c.446C > T | p.P149L |
| IQSEC3 | 12 | 250285 | 0.69 | Intron | T | C | | |
| WNK1 | 12 | 1018024 | 0.52 | 3′UTR | T | G | | |
| PRMT8 | 12 | 3649787 | 1 | Missense_Mutation | T | C | c.91T > C | p.S31P |
| PTMS | 12 | 6879650 | 0.61 | 3′UTR | T | G | | |
| PTMS | 12 | 6879662 | 0.98 | 3′UTR | T | G | | |
| LAG3 | 12 | 6881952 | 0.68 | 5′UTR | A | C | | |
| C12orf60 | 12 | 14975932 | 0.66 | Missense_Mutation | T | G | c.63T > G | p.F21L |
| KIF21A | 12 | 39705411 | 0.21 | Intron | A | C | | |
| PCED1B | 12 | 47629658 | 0.17 | Missense_Mutation | C | A | c.812C > A | p.P271H |
| RAB5B | 12 | 56380682 | 0.87 | 5′UTR | T | C | | |
| RDH16 | 12 | 57345813 | 0.54 | Nonstop_Mutation | T | G | c.954A > C | p.*318C |
| TMEM5 | 12 | 64196045 | 0.1 | Silent | C | T | c.603C > T | p.L201L |
| NAV3 | 12 | 78571071 | 0.64 | Missense_Mutation | A | C | c.5275A > C | p.K1759Q |
| PPFIA2 | 12 | 81671191 | 0.46 | Missense_Mutation | G | T | c.3215C > A | p.T1072K |
| PPFIA2 | 12 | 81671194 | 0.42 | Splice_Site | C | T | | |
| RASSF9 | 12 | 86199652 | 0.14 | Missense_Mutation | G | A | c.136C > T | p.R46C |
| POLR3B | 12 | 106820982 | 0.32 | Missense_Mutation | C | T | c.1109C > T | p.S370F |
| RP11-556N21.1 | 13 | 25144833 | 0.43 | RNA | A | G | | |
| TDRD3 | 13 | 60971461 | 0.61 | Intron | A | C | | |
| TFDP1 | 13 | 114240102 | 0.3 | 5′UTR | C | T | | |
| HSPA2 | 14 | 65008372 | 1 | Missense_Mutation | G | A | c.805G > A | p.A269T |
| ELMSAN1 | 14 | 74185939 | 0.92 | 3′UTR | A | C | | |
| SPTLC2 | 14 | 78036825 | 0.22 | Nonsense_Mutation | C | A | c.658G > T | p.E220* |
| RP11-96O20.2 | 15 | 45848224 | 0.55 | lincRNA | G | T | | |
| DUT | 15 | 48634301 | 0.41 | 3′UTR | G | A | | |
| MNS1 | 15 | 56736654 | 0.53 | Missense_Mutation | T | G | c.674A > C | p.E225A |
| SIN3A | 15 | 75706577 | 0.99 | Missense_Mutation | G | C | c.442C > 6 | p.L148V |
| CREBBP | 16 | 3779204 | 0.48 | Silent | C | G | c.5844G > C | p.P1948P |
| COG7 | 16 | 23457283 | 0.21 | Splice_Site | C | T | | |
| NPIPB9 | 16 | 28763851 | 0.06 | 5′UTR | T | C | | |
| CORO1A | 16 | 30199933 | 1 | Intron | A | G | | |
| CORO1A | 16 | 30399937 | 1 | Intron | T | G | | |
| CORO1A | 16 | 30199942 | 1 | Intron | T | G | | |
| SETD1A | 16 | 30990536 | 0.69 | Silent | T | C | c.3429T > C | p.P1143P |
| BCL6B | 17 | 6927768 | 0.31 | Silent | A | C | c.450A > C | p.P150P |
| BCL6B | 17 | 6927777 | 0.45 | Silent | A | C | c.459A > C | p.P153P |
| PFAS | 17 | 8151409 | 1 | 5′Flank | T | G | | |
| PFAS | 17 | 8172087 | 0.08 | Missense_Mutation | G | T | c.3619G > T | p.A1207S |
| RP11-219A1S.4 | 17 | 16722846 | 0.66 | RNA | G | A | | |
| RP11-744K17.9 | 17 | 23904125 | 0.11 | lincRNA | G | A | | |
| NF1 | 17 | 29422162 | 1 | 5′UTR | T | C | | |
| HNF1B | 17 | 36104902 | 0.69 | 5′UTR | T | G | | |
| HNF1B | 17 | 36104904 | 1 | 5′UTR | A | G | | |
| HNF1B | 17 | 36104910 | 1 | 5′UTR | T | G | | |
| HNF1B | 17 | 36104914 | 1 | 5′UTR | T | G | | |
| MSL1 | 17 | 38289899 | 0.23 | Nonsense_Mutation | G | T | c.1669G > T | p.E557* |
| SP6 | 17 | 45924796 | 0.2 | Missense_Mutation | T | G | c.1000A > C | p.K334Q |
| HOXB2 | 17 | 46622286 | 0.64 | 5′UTR | T | G | | |
| UTP18 | 17 | 49340654 | 0.4 | Missense_Mutation | C | G | c.362C > G | p.S121W |
| MTMR4 | 17 | 56584217 | 0.31 | Missense_Mutation | G | A | c.878C > T | p.A293V |
| ENTHD2 | 17 | 79203046 | 0.87 | Silent | T | G | c.1260A > C | p.P420P |
| HRH4 | 18 | 22057482 | 0.51 | Missense_Mutation | A | C | c.1129A > C | p.K377Q |
| REXO1 | 19 | 1827048 | 0.38 | Silent | T | G | c.1740A > C | p.P580P |
| AES | 19 | 3056403 | 1 | Intron | T | G | | |
| TUBB4A | 19 | 6495887 | 0.07 | Missense_Mutation | T | C | c.623A > G | p.Y208C |
| ZNF627 | 19 | 11728631 | 0.74 | Missense_Mutation | A | C | c.1313A > C | p.E438A |
| ZNF791 | 19 | 12739215 | 0.37 | Missense_Mutation | A | C | c.872A > C | p.E291A |
| CPAMD8 | 19 | 17006740 | 0.11 | Intron | G | A | | |
| NXNL1 | 19 | 17566477 | 1 | Silent | G | C | c.618C > G | p.G206G |
| NXNL1 | 19 | 17566484 | 1 | Missense_Mutation | T | C | c.611A > G | p.E204G |
| SLC5A5 | 19 | 17983031 | 1 | 5′UTR | A | C | | |
| KMT2B | 19 | 36224209 | 0.74 | Silent | G | C | c.6759G > C | p.P2253P |
| KMT2B | 19 | 36224215 | 0.5 | Silent | G | C | c.6765G > C | p.P2255P |
| ZNF850 | 19 | 37253563 | 0.32 | 5′UTR | A | C | | |
| CYP2A13 | 19 | 41601920 | 0.71 | 3′UTR | A | G | | |
| CIC | 19 | 42799059 | 0.3 | Missense_Mutation | C | T | c.4543C > T | p.R1515C |
| PHLDB3 | 19 | 43983726 | 0.63 | Missense_Mutation | T | G | c.1505A > C | p.H502P |
| PHLDB3 | 19 | 43983731 | 0.89 | Silent | T | G | c.1500A > C | p.P500P |
| PHLDB3 | 19 | 43983736 | 0.93 | Missense_Mutation | T | G | c.1495A > C | p.T499P |
| ZNF525 | 19 | 53887191 | 0.15 | IGR | T | A | | |
| PLCB4 | 20 | 9319601 | 0.62 | Missense_Mutation | C | T | c.286C > T | p.R96W |
| FAM182B | 20 | 25755527 | 0.27 | Silent | G | A | c.429C > T | p.S143S |
| FRG1B | 20 | 29614275 | 0.41 | 5′UTR | G | A | | |
| FRG1B | 20 | 29633900 | 0.1 | Missense_Mutation | A | G | c.539A > G | p.E180G |
| B4GALT5 | 20 | 48257072 | 0.29 | Missense_Mutation | T | G | c.737A > C | p.Y246S |
| VAPB | 20 | 56964368 | 0.39 | 5′UTR | A | C | | |
| TPTE | 21 | 11029682 | 0.11 | 5′UTR | G | A | | |
| BAGE2 | 21 | 11038748 | 0.17 | RNA | C | T | | |
| BAGE2 | 21 | 11058353 | 0.2 | RNA | T | C | | |
| BAGE2 | 21 | 11098764 | 0.04 | RNA | G | A | | |
| SMIM11 | 21 | 35751748 | 0.34 | 5′UTR | T | G | | |
| TMPRSS3 | 21 | 43815505 | 0.12 | Missense_Mutation | C | T | c.22G > A | p.AS8T |
| AIRE | 21 | 45709677 | 0.07 | Missense_Mutation | G | T | c.790G > T | p.A264S |
| KRTAP10-11 | 21 | 46066486 | 0.5 | Silent | C | T | c.111C > T | p.C37C |
| AC008132.13 | 22 | 18844763 | 0.15 | 3′UTR | T | C | | |
| POM121L4P | 22 | 21044816 | 0.05 | RNA | G | A | | |
| CHCHD10 | 22 | 24108456 | 0.58 | Missense_Mutation | T | G | c.268A > C | p.T90P |
| SMARCB1 | 22 | 24176559 | 0.59 | 3′UTR | A | C | | |
| CSNK1E | 22 | 38757479 | 0.11 | 5′UTR | A | G | | |
| EFCAB6 | 22 | 44083353 | 0.42 | Missense_Mutation | A | T | c.1140T > A | p.N380K |
| PHF21B | 22 | 45309895 | 0.58 | Missense_Mutation | A | G | c.638T > C | p.L213P |
| TLR7 | X | 12906275 | ND | Missense_Mutation | G | A | c.2648G > A | p.R883H |
| BCOR | X | 39921456 | ND | Missense_Mutation | C | T | c.4364G > A | p.R1455K |
| Unknown | X | 47658044 | ND | IGR | T | G | | |
| TGIF2LX | X | 89177570 | ND | Missense_Mutation | G | T | c.486G > T | p.L162F |
| DCAF12L1 | X | 125686202 | ND | Silent | G | A | c.390C > T | p.I130I |
| L1CAM | X | 153141379 | ND | 5′UTR | C | G | | |
| L1CAM | X | 153141386 | ND | 5′UTR | T | G | | |
| L1CAM | X | 153141401 | ND | Splice_Site | T | G | | |

MGH54

| | | | | | | | | |
|---|---|---|---|---|---|---|---|---|
| PLCH2 | 1 | 2435352 | 0.69 | Intron | T | C | | |
| PLCH2 | 1 | 2435357 | 0.69 | Intron | A | C | | |
| CEP85 | 1 | 26566306 | 0.7 | Missense_Mutation | G | A | c.32G > A | p.G11E |
| OSBPL9 | 1 | 52226257 | 0.34 | Intron | T | G | | |
| LRP8 | 1 | 53793514 | 0.08 | Missense_Mutation | A | T | c.71T > A | p.L24Q |
| DOCK7 | 1 | 62941517 | 0.06 | Missense_Mutation | A | C | c.5729T > G | p.F1910C |
| RP11-417J8.6 | 1 | 142635475 | 0.09 | lincRNA | T | G | | |
| Unknown | 1 | 144619403 | 0.08 | IGR | A | G | | |
| PMVK | 1 | 154897570 | 0.37 | 3′UTR | T | C | | |
| THBS3 | 1 | 155167452 | 0.22 | Splice_Site | T | G | | |
| KIAA0907 | 1 | 155887387 | 0.37 | Missense_Mutation | T | G | c.1343A > C | p.Q448P |
| KIAA0907 | 1 | 155887393 | 0.51 | Missense_Mutation | T | G | c.1337A > C | p.Q446P |
| SH2D2A | 1 | 156777059 | 0.37 | Missense_Mutation | C | G | c.1081G > C | p.A361P |
| SH2D2A | 1 | 156777070 | 0.38 | Missense_Mutation | T | G | c.1070A > C | p.Q357P |
| LRRC71 | 1 | 156893843 | 0.23 | Missense_Mutation | A | C | c.263A > C | p.H88P |
| VANGL2 | 1 | 160395211 | 1 | 3′UTR | A | G | | |
| VANGL2 | 1 | 160395221 | 1 | 3′UTR | A | G | | |
| CPSF3 | 2 | 9599742 | 0.27 | Missense_Mutation | G | A | c.1781G > A | p.R594K |
| CTNNA2 | 2 | 80136918 | 0.37 | Missense_Mutation | A | C | c.1051A > C | p.N351H |
| ZEB2 | 2 | 145146471 | 0.11 | 3′UTR | T | A | | |
| GTF3C3 | 2 | 197657782 | 0.06 | Silent | C | T | c.309G > A | p.E103E |
| EEF1B2 | 2 | 207025358 | 0.06 | Missense _Mutation | A | G | c.127A > G | p.S43G |
| EEF1B2 | 2 | 207025366 | 0.06 | Silent | G | A | c.135G > A | p.P45P |
| CPO | 2 | 207833951 | 0.19 | Missense_Mutation | T | G | c.916T > G | p.S306A |
| IDH1 | 2 | 209113112 | 1 | Missense_Mutation | C | T | c.395G > A | p.R132H |
| AC131097.3 | 2 | 242946237 | 0.03 | RNA | G | C | | |
| NR2C2 | 3 | 15084335 | 0.67 | Intron | T | G | | |
| ZBTB47 | 3 | 42700699 | 0.21 | Missense_Mutation | G | C | c.8526 > C | p.E284D |
| PLXNB1 | 3 | 48461613 | 0.25 | Silent | T | G | c.2082A > C | p.P694P |
| FAM86DP | 3 | 75475709 | 0.06 | RNA | T | C | | |
| EFCAB12 | 3 | 129120540 | 0.06 | Missense_Mutation | C | G | c.1615G > C | p.V539L |
| PIK3CB | 3 | 138433351 | 0.31 | Missense_Mutation | T | G | c.1261A > C | p.N421H |
| IQCJ-SCHIP1 | 3 | 159482850 | 0.09 | Missense_Mutation | G | A | c.601G > A | p.E201K |
| OTOP1 | 4 | 4228226 | 0.18 | Silent | G | A | c.366C > T | p.R122R |
| LGI2 | 4 | 25005792 | 0.94 | Missense_Mutation | C | T | c.919G > A | p.E307K |
| USP46 | 4 | 53522601 | 0.55 | Intron | C | G | | |
| PDGFRA | 4 | 55131029 | 0.16 | Intron | A | T | | |
| PDLIM5 | 4 | 95508331 | 0.95 | Intron | A | C | | |
| ZNF827 | 4 | 146744679 | 0.19 | Splice_Site | T | G | | |
| KLHL2 | 4 | 166199030 | 0.38 | Intron | A | G | | |
| SDHA | 5 | 228257 | 0.08 | Intron | T | G | | |
| CCT5 | 5 | 10250663 | 0.67 | Intron | A | G | | |
| C5orf51 | 5 | 41909846 | 0.37 | Splice_Site | A | T | | |
| KIF2A | 5 | 61602215 | 0.65 | 5′UTR | T | C | | |
| KIF2A | 5 | 61602219 | 1 | 5′UTR | A | C | | |
| SNRNP48 | 6 | 7609118 | 0.69 | 3′UTR | G | T | | |
| BMP6 | 6 | 7727541 | 0.08 | Missense_Mutation | A | T | c.353A > T | p.Q118L |
| TFAP2A | 6 | 10402545 | 0.24 | Intron | T | G | | |
| CASC14 | 6 | 22136876 | 0.72 | lincRNA | T | G | | |
| LRRC16A | 6 | 25551276 | 0.58 | Silent | T | C | c.2467T > C | p.L823L |
| SCAND3 | 6 | 28543205 | 1 | Missense_Mutation | G | A | c.1277C > T | p.T426I |
| ZNRD1-AS1 | 6 | 29977327 | 0.07 | RNA | T | C | | |
| NRM | 6 | 30658764 | 0.34 | 5′UTR | A | G | | |
| NRM | 6 | 30658769 | 0.32 | 5′UTR | T | G | | |
| RNF5 | 6 | 32147865 | 0.07 | Missense_Mutation | C | T | c.407C > T | p.T136I |
| RGL2 | 6 | 33269389 | 0.73 | 5′Flank | T | G | | |
| TTK | 6 | 80717709 | 0.13 | Missense_Mutation | G | T | c.323G > T | p.S108I |
| ORC3 | 6 | 88318940 | 1 | Missense_Mutation | A | C | c.706A > C | p.I236L |
| COQ3 | 6 | 99819447 | 0.31 | Missense_Mutation | A | C | c.746T > G | p.F249C |
| SOBP | 6 | 107955437 | 0.23 | Silent | G | C | c.1389G > C | p.P463P |
| SEC63 | 6 | 108214765 | 0.07 | Nonsense_Mutation | A | T | c.1595T > A | p.L532* |
| VNN1 | 6 | 133014444 | 0.57 | Missense_Mutation | A | C | c.545T > G | p.F182C |
| INTS1 | 7 | 1526685 | 0.06 | Missense_Mutation | C | T | c.2699G > A | p.G900D |
| SP4 | 7 | 21467806 | 0.64 | 5′UTR | G | C | | |
| WIPF3 | 7 | 29874364 | 0.68 | Silent | A | C | c.24A > C | p.P8P |
| WIPF3 | 7 | 29874367 | 0.84 | Silent | T | C | c.27T > C | p.P9P |
| PTPRZ1 | 7 | 121651723 | 0.9 | Nonsense_Mutation | C | T | c.2623C > T | p.Q875* |
| TRIM24 | 7 | 138145895 | 0.06 | Intron | C | T | | |
| PRSS1 | 7 | 142459042 | 0.22 | Intron | C | T | | |
| RP11-481A20.11 | 8 | 11872530 | 0.09 | Missense_Mutation | G | A | c.29C > T | p.A10V |
| RP11-481A20.11 | 8 | 11872550 | 0.09 | Missense_Mutation | G | C | c.9C > G | p.S3R |
| PDLIM2 | 8 | 22447026 | 0.49 | Intron | A | C | | |
| ZNF395 | 8 | 28210808 | 0.34 | Missense_Mutation | T | G | c.701A > C | p.H234P |
| ASPH | 8 | 62491435 | 0.07 | Intron | C | T | | |
| CHMP4C | 8 | 82665470 | 0.31 | Missense_Mutation | A | C | c.362A > C | p.E121A |
| SUFU | 10 | 104263946 | 0.29 | Missense_Mutation | A | C | c.37A > C | p.T13P |
| SUFU | 10 | 104263957 | 0.29 | Silent | G | C | c.48G > C | p.P16P |
| CALHM2 | 10 | 105209523 | 0.04 | Missense_Mutation | G | A | c.176C > T | p.A59V |
| CALY | 10 | 135137975 | 0.33 | IGR | T | G | | |
| CALY | 10 | 135137979 | 0.38 | IGR | C | G | | |
| TSSC2 | 11 | 3424149 | 0.06 | RNA | C | T | | |
| BTBD10 | 11 | 13435092 | 0.19 | Missense_Mutation | T | G | c.793A > C | p.K265Q |
| TRIM48 | 11 | 55035844 | 0.08 | Missense_Mutation | T | C | c.574T > C | p.Y192H |
| RPLP0P2 | 11 | 61405030 | 0.15 | RNA | T | A | | |
| DNAJC4 | 11 | 64000291 | 0.56 | Missense_Mutation | C | T | c.481C > T | p.L161F |
| FOLH1B | 11 | 89395322 | 0.15 | RNA | C | T | | |
| STT3A | 11 | 125476327 | 0.29 | Silent | A | C | c.747A > C | p.I249I |
| PTMS | 12 | 6879650 | 0.37 | 3′UTR | T | G | | |
| PTMS | 12 | 6879653 | 0.68 | 3′UTR | A | G | | |
| PTMS | 12 | 6879656 | 0.58 | 3′UTR | T | G | | |
| FAM90A1 | 12 | 8380196 | 0.17 | 5′UTR | A | G | | |
| RDH16 | 12 | 57345813 | 0.43 | Nonstop_Mutation | T | G | c.954A > C | p.*318C |
| DTX3 | 12 | 58001051 | 0.4 | Silent | T | C | c.405T > C | p.A135A |
| NAV3 | 12 | 78571071 | 0.33 | Missense _Mutation | A | C | c.5275A > C | p.K1759Q |
| APAF1 | 12 | 99117444 | 0.18 | Missense_Mutation | G | A | c.3232G > A | p.E1078K |
| SETD1B | 12 | 122261027 | 0.26 | Silent | A | C | c.4542A > C | p.P1514P |
| RP11-556N21.1 | 13 | 25168489 | 0.14 | RNA | G | A | | |
| ESD | 13 | 47345484 | 0.53 | 3′UTR | G | T | | |
| TDRD3 | 13 | 60971461 | 0.61 | Intron | A | C | | |
| TDRD3 | 13 | 60971466 | 0.61 | Intron | A | C | | |
| COL4A1 | 13 | 110833688 | 0.06 | Missense_Mutation | C | T | c.2144G > A | p.R715H |
| OR4Q3 | 14 | 20216484 | 0.25 | Missense_Mutation | A | C | c.898A > C | p.K300Q |
| TM9SF1 | 14 | 24661303 | 0.86 | Intron | C | G | | |
| GPX2 | 14 | 65406817 | 0.42 | Intron | G | T | | |
| CALM1 | 14 | 90870229 | 0.66 | Missense_Mutation | G | A | c.202G > A | p.E68K |
| Unknown | 14 | 106134738 | 0.05 | IGR | T | C | | |
| HERC2 | 15 | 28459392 | 0.06 | Missense_Mutation | G | A | c.6385C > T | p.R2129C |
| LPCAT4 | 15 | 34659245 | 0.25 | Silent | T | G | c.57A > C | p.P19P |
| WDR72 | 15 | 53994476 | 0.69 | Missense_Mutation | G | A | c.1424C > T | p.S475L |
| MNS1 | 15 | 56736654 | 0.24 | Missense_Mutation | T | G | c.674A > C | p.E225A |
| CLN6 | 15 | 68500436 | 0.52 | 3′UTR | A | C | | |
| CYP1A2 | 15 | 75045612 | 0.81 | Splice_Site | G | A | | |
| TSC2 | 16 | 2121833 | 0.12 | Silent | T | C | c.1995T > C | p.P665P |
| CREBBP | 16 | 3779210 | 0.38 | Silent | T | G | c.5838A > C | p.P1946P |
| GRIN2A | 16 | 10273739 | 0.98 | Intron | A | C | | |
| PFAS | 17 | 8151415 | 0.9 | 5′Flank | T | G | | |
| RP11-744K17.9 | 17 | 21904093 | 0.19 | lincRNA | A | G | | |
| TLCB1 | 17 | 27051858 | 0.29 | Silent | A | G | c.414T > C | p.G138G |
| HNF1B | 17 | 36104904 | 0.85 | 5′UTR | A | G | | |
| HNF1B | 17 | 36104910 | 0.62 | 5′UTR | T | G | | |
| HNF1B | 17 | 36104914 | 0.69 | 5′UTR | T | G | | |
| WNK4 | 17 | 40946930 | 0.18 | Missense_Mutation | A | C | c.2491A > C | p.I831L |
| WNK4 | 17 | 40946954 | 0.27 | Missense_Mutation | A | C | c.2515A > C | p.S839R |
| WNK4 | 17 | 40946965 | 0.29 | Silent | A | C | c.2526A > C | p.P842P |
| ITGA2B | 17 | 42452325 | 0.21 | Intron | G | C | | |
| SP6 | 17 | 45924796 | 0.12 | Missense_Mutation | T | G | c.1000A > C | p.K334Q |
| HOXB2 | 17 | 46622302 | 1 | 5′UTR | T | G | | |
| WBP2 | 17 | 73851262 | 0.59 | Intron | G | C | | |
| USP36 | 17 | 76799999 | 0.42 | Missense_Mutation | T | G | c.2278A > C | p.T760P |
| C1QTNF1 | 17 | 77021988 | 0.1 | 5′UTR | T | C | | |
| AATK | 17 | 79093349 | 0.62 | Silent | C | T | c.3915G > A | p.P1305P |
| ENTHD2 | 17 | 79203046 | 0.57 | Silent | T | G | c.1260A > C | p.P420P |
| EPG5 | 18 | 43534623 | 1 | Nonsense_Mutation | G | A | c.745C > T | p.Q249* |
| SMARCA4 | 19 | 11132437 | 0.78 | Missense_Mutation | C | T | c.2653C > T | p.R885C |
| SMARCA4 | 19 | 11132513 | 0.04 | Missense_Mutation | C | T | c.2729C > T | p.T910M |
| ZNF627 | 19 | 11728631 | 0.63 | Missense_Mutation | A | C | c.1313A > C | p.E438A |
| BRD4 | 19 | 15353841 | 1 | Silent | T | G | c.3039A > C | p.P1013P |
| CPAMD8 | 19 | 17006740 | 0.06 | Intron | G | A | | |
| NXNL1 | 19 | 17566481 | 0.89 | Missense_Mutation | T | C | c.614A > G | p.E205G |
| NXNL1 | 19 | 17566484 | 0.52 | Missense_Mutation | T | C | c.611A > 6 | p.E2046 |
| C19orf60 | 19 | 18702255 | 0.81 | Intron | C | T | | |
| Unknown | 19 | 34583535 | 0.53 | IGR | T | C | | |
| CYP2A13 | 19 | 41601925 | 0.34 | 3′UTR | C | G | | |
| CIC | 19 | 42796236 | 0.69 | Splice_Site | A | G | | |
| ARHGAP35 | 19 | 47440657 | 0.32 | Missense_Mutation | A | C | c.3818A > C | p.E1273A |
| FUZ | 19 | 50310295 | 0.11 | 3′UTR | T | C | | |
| SIRPB1 | 20 | 1585397 | 0.18 | Intron | T | C | | |
| OCSTAMP | 20 | 45170141 | 0.04 | Silent | G | A | c.1473C > T | p.T491T |
| B4GALT5 | 20 | 48257072 | 0.2 | Missense_Mutation | T | G | c.737A > C | p.Y246S |
| VAPB | 20 | 56964377 | 0.33 | 5′UTR | A | C | | |
| MIS18A | 21 | 33641263 | 0.4 | 3′UTR | G | T | | |
| PI4KA | 22 | 21064203 | 0.04 | Missense_Mutation | G | A | c.5992C > T | p.L1998F |
| CHCHD10 | 22 | 24108440 | 0.22 | Missense_Mutation | T | G | c.284A > C | p.Q95P |
| Unknown | 22 | 25053920 | 0.04 | IGR | C | T | | |
| TTC28 | 22 | 28692203 | 0.08 | Missense_Mutation | T | G | c.916A > C | p.K306Q |
| BIK | 22 | 43524599 | ND | Silent | A | C | c.358A > C | p.R120R |
| IQSEC2 | X | 53296215 | ND | Intron | C | A | | |
| MSN | X | 64956699 | ND | Silent | G | A | c.1002G > A | p.E334E |
| LONRF3 | X | 118143186 | ND | Missense_Mutation | A | C | c.1628A > C | p.E543A |
| MAGEA4 | X | 151091946 | ND | 5′UTR | C | T | | |
| GABRQ | X | 151815566 | ND | Missense_Mutation | A | C | c.464A > C | p.D155A |
| ARHGAP4 | X | 153175924 | ND | Intron | T | C | | |

MGH60

| | | Start_ | | Variant_ | Tumor_ | Tumor_ | cDNA_ | Protein_ |
|---|---|---|---|---|---|---|---|---|
| Hugo_Symbol | Chromosome | position | ccf_hat | Classification | Seq_Allele1 | Seq_Allele2 | Change | Change |
| MST1L | 1 | 17084569 | NA | RNA | G | A | | |
| PADI3 | 1 | 17596854 | 1 | Missense_Mutation | G | A | c.779G > A | p.G260D |
| LCE1A | 1 | 152799991 | 0.18 | Missense_Mutation | A | C | c.43A > C | p.K15Q |
| LCE1A | 1 | 152800003 | 0.17 | Missense_Mutation | A | C | c.55A > C | p.K19Q |
| PMVK | 1 | 154897570 | 0.56 | 3′UTR | T | C | | |
| THBS3 | 1 | 155167452 | 0.43 | Splice_Site | T | G | | |
| SH2D2A | 1 | 156777070 | 0.26 | Missense_Mutation | T | G | c.1100A > C | p.Q367P |
| APCS | 1 | 159558233 | 0.04 | Missense_Mutation | A | G | c.407A > G | p.K136R |
| PPP1R12B | 1 | 202407176 | 0.05 | Silent | G | A | c.1482G > A | p.G494G |
| LAMB3 | 1 | 209797025 | 0.02 | Missense_Mutation | G | C | c.2183C > G | p.A728G |
| SMYD3 | 1 | 246093457 | 0.24 | Intron | T | C | | |
| CAD | 2 | 27456266 | 0.96 | Silent | G | T | c.3078G > T | p.A1026A |
| GGCX | 2 | 85776973 | 0.21 | 3′UTR | G | A | | |
| ANKRD36 | 2 | 97869931 | 0.14 | Missense_Mutation | A | T | c.2992A > T | p.T998S |
| TMEM182 | 2 | 103378601 | 0.53 | 5′UTR | G | T | | |
| KIF5C | 2 | 149633155 | 0.49 | 5′UTR | A | C | | |
| XIRP2 | 2 | 168103475 | 0.37 | Missense_Mutation | C | T | c.5573C > T | p.T1858M |
| PGAP1 | 2 | 197791356 | 0.1 | 5′UTR | G | A | | |
| FASTKD2 | 2 | 207632128 | 1 | Silent | C | T | c.711C > T | p.H237H |
| IDH1 | 2 | 209113112 | 0.84 | Missense_Mutation | C | T | c.395G > A | p.R132H |
| NGLY1 | 3 | 25770654 | 0.16 | Silent | T | G | c.1527A > C | p.I509I |
| SUCLG2 | 3 | 67559234 | 0.26 | Missense_Mutation | G | T | c.754C > A | p.Q252K |
| CHMP2B | 3 | 87303046 | 0.24 | 3′UTR | C | A | | |
| GPR31 | 6 | 167571126 | 0.16 | Missense_Mutation | G | A | c.194C > T | p.A65V |
| ZNF395 | 8 | 28210802 | 0.26 | Missense_Mutation | T | G | c.707A > C | p.Q236P |
| COL22A1 | 8 | 139824118 | 0.53 | Missense_Mutation | T | G | c.1373A > C | p.Q458P |
| SEMA4D | 9 | 92003803 | 0.99 | Missense_Mutation | G | C | c.934C > G | p.L312V |
| C10orf112 | 10 | 19981478 | 1 | Silent | A | G | c.4260A > G | p.P1420P |
| SVILP1 | 10 | 30986357 | 0.06 | RNA | T | C | | |
| ANKRD30A | 10 | 37431050 | 0.06 | Missense_Mutation | G | C | c.1057G > C | p.A353P |
| PTEN | 10 | 89720659 | 0.23 | Missense_Mutation | G | T | c.810G > T | p.M270I |
| RRP12 | 10 | 99118376 | 0.84 | Splice_Site | T | C | c.3708_splic [symbol] | p.K1237_spli [symbol] |
| AFAP1L2 | 10 | 116059958 | 0.94 | Missense_Mutation | C | T | c.1952G > A | p.S651N |
| ZNF511 | 10 | 135137975 | 0.36 | Intron | T | G | | |
| MRVI1 | 11 | 10647847 | 0.07 | Missense_Mutation | G | A | c.761C > T | p.P254L |
| BTBD10 | 11 | 13435092 | 0.18 | Missense_Mutation | T | G | c.793A > C | p.K265Q |
| OR5AK2 | 11 | 56757259 | 0.53 | Missense_Mutation | A | C | c.871A > C | p.S291R |
| DLG2 | 11 | 83252723 | 0.87 | Splice Site | A | C | | |
| CCBC81 | 11 | 86133688 | 0.09 | Silent | C | T | c.1095C > T | p.T365T |
| NPAT | 11 | 108031631 | 0.88 | Missense_Mutation | T | C | c.4182A > G | p.I1394M |
| PTS | 11 | 112099324 | 0.29 | Silent | C | T | c.91C > T | p.L31L |
| ESAM | 11 | 124623472 | 1 | 3′UTR | C | T | | |
| STT3A | 11 | 125476327 | 0.23 | Silent | A | C | c.747A > C | p.I249I |
| WNK1 | 12 | 1018024 | 0.36 | 3′UTR | T | G | | |
| PTMS | 12 | 6879662 | 0.39 | 3′UTR | T | G | | |
| LINC00937 | 12 | 8549081 | 0.14 | lincRNA | C | G | | |
| BICD1 | 12 | 32481354 | 0.82 | Silent | G | A | c.1965G > A | p.A655A |
| RPAP3 | 12 | 48096569 | 0.81 | Nonsense_Mutation | C | A | c.55G > T | p.E19* |
| TIMELESS | 12 | 56818562 | 0.89 | Missense_Mutation | G | A | c.1849C > T | p.L617F |
| RDH16 | 12 | 57345813 | 0.16 | Nonstop_Mutation | T | G | c.954A > C | p.*318C |
| NAV3 | 12 | 78571071 | 0.34 | Missense_Mutation | A | C | c.5275A > C | p.K1759Q |
| SLC8B1 | 12 | 113756885 | 1 | Intron | G | A | | |
| PDS5B | 13 | 33332227 | 0.48 | Missense_Mutation | G | T | c.3059G > T | p.C1020F |
| PDS5B | 13 | 33332229 | 0.47 | Missense_Mutation | C | T | c.3061C > T | p.L1021F |
| RP11-483E23.2 | 15 | 28599954 | 0.02 | RNA | A | G | | |
| CHRNE | 17 | 4802379 | 1 | Missense_Mutation | C | T | c.1243G > A | p.A415T |
| BCL6B | 17 | 6927768 | 0.3 | Silent | A | C | c.450A > C | p.P150P |
| CYP2A13 | 19 | 41601907 | 0.31 | 3′UTR | C | G | | |
| CYP2A13 | 19 | 41601920 | 0.23 | 3′UTR | A | G | | |
| CYP2A13 | 19 | 41601925 | 0.28 | 3′UTR | C | G | | |
| CIC | 19 | 42793757 | 1 | Missense_Mutation | C | T | c.3370C > T | p.R1124W |
| VAPB | 20 | 56964377 | 0.18 | 5′UTR | A | C | | |
| POM121L4P | 22 | 21044374 | 0.17 | RNA | G | C | | |
| PPM1F | 22 | 22277819 | 0.93 | Silent | C | T | c.507G > A | p.V169V |
| AR | X | 66765161 | | Missense_Mutation | A | T | c.173A > T | p.Q58L |
| IGBP1 | X | 69354420 | | Missense_Mutation | T | G | c.236T > G | p.L79R |
| SAGE1 | X | 134989127 | | Missense_Mutation | A | G | c.779A > G | p.K260R |
| MECP2 | X | 153296115 | | Silent | T | G | c.1164A > C | p.P388P |

[symbol]

indicates data missing or illegible when filed

Finally, to explore point mutations with an additional strategy, independent of single cell RNA-seq, Applicants also tested specific mutations in single cells by mutation-sensitive qPCR (Methods). While most subclonal mutations were of unknown functional relevance, Applicants were intrigued by the identification of a subclonal CIC mutation in MGH53 (˜30% frequency by ABSOLUTE). CIC is a known tumor suppressor in oligodendroglioma (115), and this missense p. R1515C mutation, also observed in four patients in the TCGA cohort (112) (the second most common across 66 patients with any CIC mutation). CIC is haploid (as it is coded on chromosome 19q) and thus allows us to ascertain both mutant and WT status. Because RNA-seq reads detected the CIC mutation in only 7 of MGH53 cells, Applicants tested its presence in additional cells using a mutation-sensitive qPCR approach and were able to ascertain 28 CIC mutant cells (including validation of all 7 cells detected by RNA-seq reads) and 27 CIC wild-type MGH53 cells (FIG. 38d). Importantly, Applicants identified a signature of expression changes between the CIC mutant and WT cells (FIG. 38e, Table 22), including increased expression of the transcription factors ETV1 and ETV5, which were recently shown to be regulated by CIC (116). Despite these specific transcriptional changes that accompany tumor progression, both CIC mutant and CIC wild-type cells spanned all the tumors' subpopulations (FIG. 38d), indicating that the tumor hierarchy is maintained during clonal evolution.

TABLE 22
Genes up regulated (top) or downregulated
(bottom) in CIC-mutant cells of MGH53.
Genes in CIC-mutant

| | | |
|---|---|---|
| | CIC mutant vs. | CIC mutant vs. |
| Gene | CIC WT (log2-ratio) | unresolved (log2-ratio) |

upnregulated in CIC-mutants

| | | |
|---|---|---|
| ALG9 | 1.227 | 0.8928 |
| AP3S1 | 1.5968 | 0.7338 |
| ARRDC3 | 1.9209 | 1.4759 |
| BRAT1 | 1.4686 | 0.7514 |
| CLN3 | 1.5573 | 1.0239 |
| CNTNAP2 | 1.0757 | 0.7058 |
| COL16A1 | 1.3021 | 0.6934 |
| CTTN | 1.8597 | 1.461 |
| DLD | 1.7493 | 1.278 |
| DOCK10 | 1.1863 | 0.8959 |
| DSEL | 1.3431 | 0.9541 |
| ECI2 | 1.4268 | 0.6268 |
| EP300 | 1.05 | 0.8556 |
| ETV1 | 1.7266 | 1.3677 |
| ETV5 | 1.4806 | 1.2395 |
| FAR1 | 1.1284 | 0.6152 |
| FOXRED1 | 1.3849 | 0.6961 |
| FYTTD1 | 1.3993 | 0.7856 |
| GATS | 1.2712 | 0.7535 |
| GFRA1 | 1.1055 | 0.6877 |
| GLT25D2 | 1.8813 | 1.4116 |
| GPR56 | 1.2726 | 1.1663 |
| IGSF8 | 1.6315 | 1.2388 |
| KANK1 | 1.8026 | 1.4367 |
| KIAA1467 | 1.3175 | 0.9784 |
| KIF22 | 1.7248 | 1.1386 |
| LNX1 | 1.2214 | 0.7705 |
| LPCAT1 | 1.4064 | 0.9667 |
| ME3 | 1.3976 | 0.9663 |
| MEGF11 | 1.4456 | 0.6222 |
| MRPS16 | 1.3175 | 0.6551 |
| NAV1 | 1.3141 | 0.796 |
| NFIA | 1.2509 | 0.931 |
| NIN | 1.4232 | 0.8497 |
| NLGN3 | 1.47 | 0.8141 |
| NUP188 | 1.3793 | 0.8259 |
| PCDH15 | 1.3156 | 0.9597 |
| PCDHB9 | 1.5753 | 0.7125 |
| PPP2R2B | 1.7528 | 0.9681 |
| PPWD1 | 1.5658 | 0.7861 |
| PTN | 1.7714 | 0.8994 |
| RASD1 | 2.0831 | 0.9614 |
| RNF214 | 1.4118 | 0.9173 |
| SDC3 | 1.3395 | 0.884 |
| SEC24B | 1.2845 | 0.6596 |
| SLC38A10 | 1.3295 | 1.4766 |
| STIM1 | 1.268 | 0.9125 |
| TMEM181 | 1.3799 | 0.9492 |
| TTLL5 | 1.1704 | 0.7158 |
| VARS | 1.2929 | 0.7738 |
| YJEFN3 | 1.5865 | 0.7356 |
| ZNF451 | 1.0488 | 0.6191 |
| ZNF564 | 1.3004 | 0.9083 |

downregulated in CIC-mutants

| | | |
|---|---|---|
| ANKMY2 | −1.579 | −0.6162 |
| ATF4 | −1.9523 | −1.3151 |
| BRK1 | −1.837 | −1.9774 |
| BTF3L4 | −1.3483 | −1.0247 |
| EIF3C | −2.0108 | −0.8491 |
| EVI2A | −1.3452 | −0.8935 |
| GFAP | −2.281 | −0.82 |
| MAD2L2 | −1.5275 | −1.1485 |
| MPV17 | −1.761 | −1.2259 |
| MRPL46 | −1.6656 | −0.5991 |
| NDUFV1 | −1.8719 | −1.4593 |
| NFE2L2 | −2.1095 | −0.634 |
| RAB1A | −1.5867 | −0.9021 |
| RCOR3 | −1.261 | −0.8461 |
| RSL1D1 | −1.2432 | −0.8095 |
| TTC14 | −1.3767 | −0.727 |

Taken together, the CNV and point-mutation analyses demonstrate that various subclonal mutations span the cellular hierarchy defined by expression profiles and strongly argue that this hierarchy reflects non-genetic states. Similar results were also obtained for analysis of a loss-of-heterozygosity event in MGH54 (FIG. 57). While our genetic analysis does not cover all possible mutations due to technical limitations, Applicants note that the alternative model of genetically-driven hierarchy would predict that all subclonal mutations should conform to a global phylogenetic structure that distinguishes between tumor compartments, and is thus highly inconsistent with our results (FIG. 58). Interestingly, Applicants also identified down-regulation of GFAP in CIC mutant cells, possibly contributing to the weaker GFAP expression in oligodendrogliomas than astrocytomas (95). Despite these specific transcriptional changes, both CIC mutant and CIC wild-type cells spanned all the tumors' subpopulations (FIG. 38d), further indicating that the tumor hierarchy is maintained during clonal evolution.

While genetic events do not appear to define the hierarchy, they may nevertheless influence it. The two clones detected in MGH36 and MGH97 each included cells from all three compartments of the cellular hierarchy, yet they differed in their relative distributions (FIG. 38a,b, FIG. 55). Clone 1 of MGH36 displayed higher frequency of stem/progenitors (P=4*10⁻¹⁰, Fisher's exact test) while clone 2 displayed higher frequency of AC-like cells (P=2*10⁻¹⁰). Similarly, clone 2 of MGH97 contained higher frequency of stem/progenitors (P<10⁻¹⁶), suggesting that genetic evolution may have modulated the patterns of self-renewal and differentiation in these tumors. Furthermore, the frequencies of cycling cells were higher in clone 1 of MGH36 and in clone 2 of MGH97, consistent with their increased frequencies of stem/progenitors. In MGH36 Applicants also observed rare OC-like cells in the G1/S phases exclusively in clone 2 (FIG. 55). Thus, the coupling between cell cycle and stemness may also be partially affected by genetic events.

In conclusion, this large-scale analysis of single-cell composition in grade II gliomas uncovers a developmental hierarchy shared across multiple oligodendrogliomas and multiple genetic subclones, indicating a model of tumorigenesis where a subpopulation of stem/progenitor cells propagates these tumors in humans, while accruing new mutations, as well as giving rise to differentiated and non-cycling cells of two distinct glial lineages with similar genotypes. Indeed, this hierarchy is recapitulated in clones that are genetically distinguishable in our data, such as in CIC wild-type vs. mutant cells. Interestingly, our single-cell data indicate that oligodendroglioma stem/progenitor cells resemble a primitive tri-potent neural cell type, such as NSC or NPC, more so than a more committed glial progenitor like an OPC (108, 117).

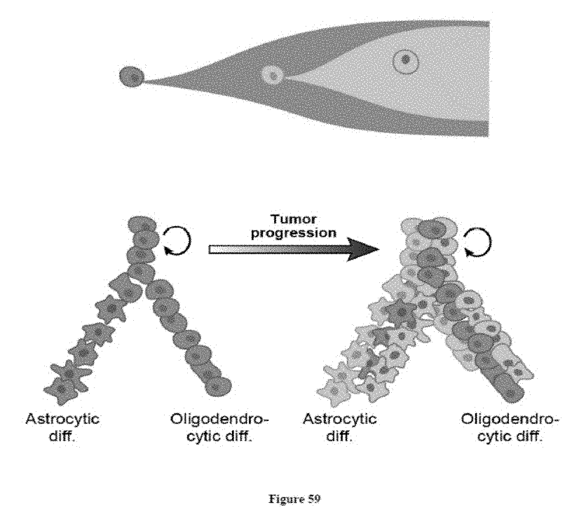

One limitation of studying low-grade oligodendrogliomas is that Applicants could neither perform functional validation of tumoral lineages nor test the capacity of different populations to initiate tumors in animals, since human grade II oligodendrogliomas do not grow in mouse xenograft assays, and even in-vitro models are sparse and maintain only limited similarity to cancer cells in situ. Yet our approach and analyses highlight the key role of single cell genomics as a tool for unbiased analysis of single-cell states directly in patient tumors, without confounding factors such as xenogeneic milieu and conditions that are drastically different from the native environment (72). Outlining genetic from non-genetic influences—albeit with limitations in sensitivity due to single cell RNA-Seq—allows us to present an integrated model of how diverse genetic clones, each with their on developmental hierarchy, coordinate tumor maintenance and evolution in humans, unifying the cancer stem cell and the genetic models of cancer in this clinical context (72) (FIG. 59).

Our results highlight a subpopulation of undifferentiated cells that possess stem cell transcriptional signatures and also show enriched proliferative potential. Thus, the most primitive and undifferentiated population of cancer cells are the main source of proliferating cells in patients with oligodendroglioma. This might explain the relative clinical sensitivity of these tumors to treatments that selectively kill proliferating cells such as radiochemotherapies (118). At least early in their pathogenesis these tumors may maintain hierarchies from normal development with stem cells that robustly follow differentiation programs, leaving oligodendroglioma stem cells as the only cycling populations. This architecture might differ in other brain tumors and in higher-grade lesions where differentiation might be compromised. By providing the genome-wide transcriptional signature of cancer stem/progenitor cells in oligodendroglioma, this work delineates cellular programs that represent valuable targets to impact tumor growth. The verticality of the observed hierarchy indicates that, in this clinical context, triggering cells to differentiate along one of two glial axes may yield therapeutic benefit. It is postulated that further studies, deploying large-scale single-cell profiling technologies in genetically defined human malignancies will demonstrate the generality of our findings and investigate opportunities for clinical translation.

Note 1. Accounting for the Impact of Technical and Batch Effects.

Applicants used several approaches to ascertain that our transcriptional signatures are observed independently of technical effects. First, different batches are indistinguishable with respect to the expression hierarchy, as shown in FIG. S9B. Second, to minimize the impact of technical effects, namely the differences in complexity (e.g. the number of genes detected per cell), Applicants use a weighted version of principal component analysis as described in Methods. Third, the biological clusters Applicants describe are not driven by complexity. As described in Methods, Applicants performed control PCA on shuffled data. Comparison of the PCA on the original and shuffled data (FIG. S4D) shows that the OC-like and AC-like genes used in our analysis lose their association with PC1 in the shuffled data, indicating that their patterns are not driven by complexity. Similarly, complexity does not account for the PC2/3 sternness program, as PC2 cell scores are positively correlated with complexity (R=0.27), while PC3 cell scores are negatively correlated with complexity (R=−0.24) and stemness genes were defined as those correlated with both PC2 and PC3.

Note 2. Assessing the Presence of Intermediate Differentiation States.

Technical noise is not expected to distinguish functionally-related from functionally-unrelated sets of genes. Within a given cell, the level of each gene can be over-estimated or under-estimated due to the capture of only a subset of transcripts and their potentially biased amplification; but there is no reason to expect that two functionally related genes will have the same pattern, i.e., commonly over-estimated or commonly under-estimated, except as correlated to their global expression levels. That is, the exception is if the two genes are both highly expressed or both lowly expressed and thus could be commonly affected by the “complexity” of single cell libraries, such that two lowly expressed genes tend to be undetected in cells with a lower overall number of detected genes. However, this does not affect our lineage scores, both because the set of AC and OC genes are not associated with very different overall expression levels, and because Applicants use “control” gene-sets with comparable expression levels when defining lineage scores. In each of the three tumors that Applicants profiled at high depth, and within each of the two lineages Applicants find significant co-expression patterns that suggest distinct differentiation states (FIG. 48). For example, within the AC lineage, Applicants find significant co-expression patterns in the range of 0.5 to 1, as well as within the range of 1 to 2. However, in more limited ranges Applicants typically do not detect significant co-expression patterns (e.g., in the range 1.5 to 2. Applicants detect significant co-expression only in one of the three tumors). Applicants conclude that cells likely exist in distinct stages of differentiation although the number of distinct states may be limited.

### Example 5

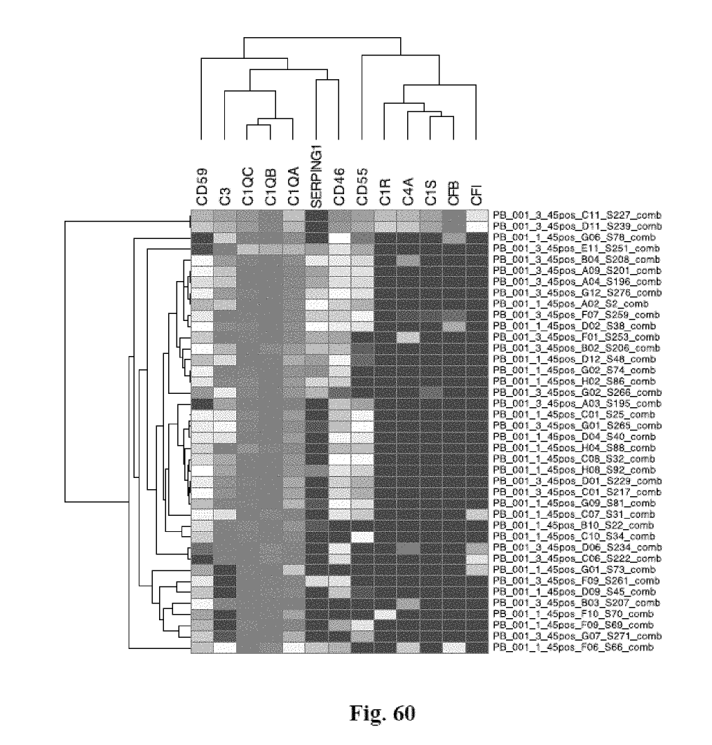

Applicants performed downstream analyses of human patient-derived single-cell RNA seq data from malignant tissue of a human patient with breast cancer metastasis in the brain. Applicants discovered correlations with complement gene signatures by analyzing the expression of CD59, C3, C1QC, C1QB, C1QA, SERPING1, CD46, CD55, C1R, C4A, C1S, CFB and CFI in microglia. T-cell, and tumor cell populations in breast metastases in the brain. Microglia strongly upregulate expression of C1q genes (FIG. 60). This is consistent with the activity of macrophage-like species to develop C1q downstream of the classical complement pathway. In particular, the genes of the C1 subunits (e.g. C1QB, C1QC, and C1R) are upregulated. Interestingly, CIS is not produced by microglia (see tumors). Microglia strongly downregulate CFB and CFI. CFI is a deregulator of the classical complement pathway by downstream enzymatic cleavage of C3b (not C3 to form C3b), CFB activates the alternative pathway, by association with C3b to form C3 convertase. This suggests that microglia in this patient are upregulating the classical vs. the alternative pathway to signal an IgG-based antibody response, leading to T cell density. Moreover, the expression pattern could suggest the possibility of activating the alternative pathway depending on the T-cell response.

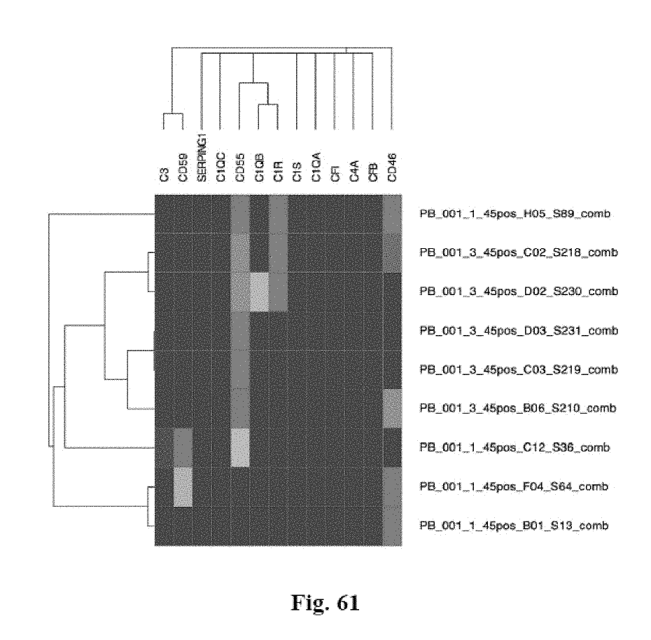

Based on the discovery that microglia may be activating the classical complement pathway, Applicants looked at the T-cell population in this patient's brain metastases (FIG. 61). In the event of metastases, it has been reported that the blood-brain barrier is compromised, allowing external cells to intravasate into the brain region of the tumor. As expected, T-cells were discovered in the CD45+ population in the resected brain metastases. Applicants confirmed T-cell identity by observing differential markers and unsupervised reduction analyses. Applicants investigated these cells with respect to the complement pathway. Approximately 9 CD45+ cells have CD8+ T-cell-specific expression. T-cells demonstrate expression of complement regulatory genes CD55, CD59, and CD46. The majority of cells express CD55, and those that do not, express CD46 or CD59, CD55 directly inhibits the formation of complement convertases, and thereby directly inhibits the formation of the attack complex (which is the primary, resultant effector of the complement pathway). This strongly suggests that T-cells infiltrating the metastases have an inhibitory role in complement activation, and could be a potential source of regulation subject to modulation, specifically in metastases.

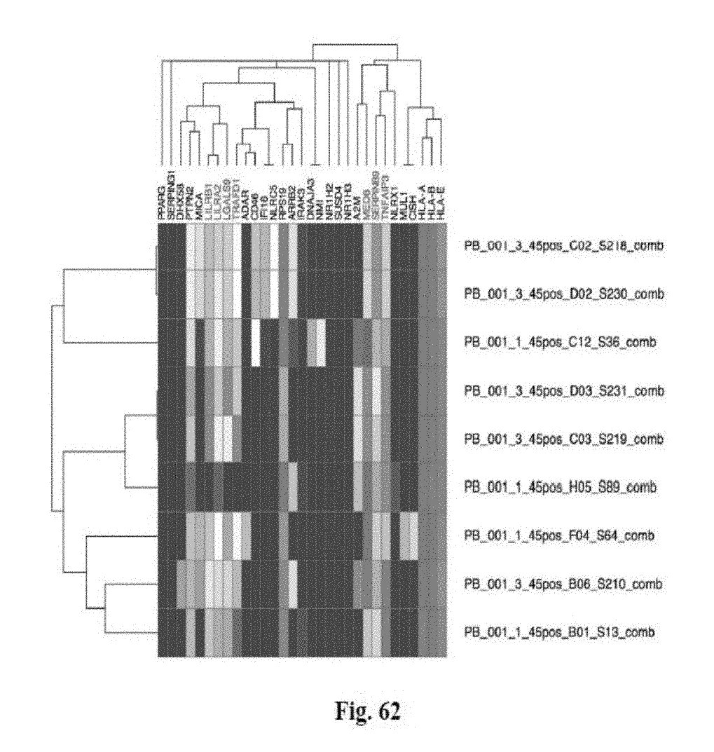

Applicants also analyzed these cells according to their expression of known immune regulatory genes (GO:0050777) (FIG. 62). The Results showed concomitant expression of MED6, SERPINB6, and TNFAIP3 which downregulate cytotoxicity in CD8+ T-cells and NK cells against tumors. Additionally, several cells (7/9) express TRAFD1 and LGALS9 which are negative feedback regulators of immune response. Finally, LILRB1/LILRA2 are expressed in a subset, which downregulate innate response and antigen binding. The data suggests that infiltrating T-cells are inhibitory to complement activation and suggests regulatory source of modulation. Not being bound by a theory, complement may recruit T cells, however the T cells have downregulated cytotoxicity. The T cells may have increased activity by activation of complement.

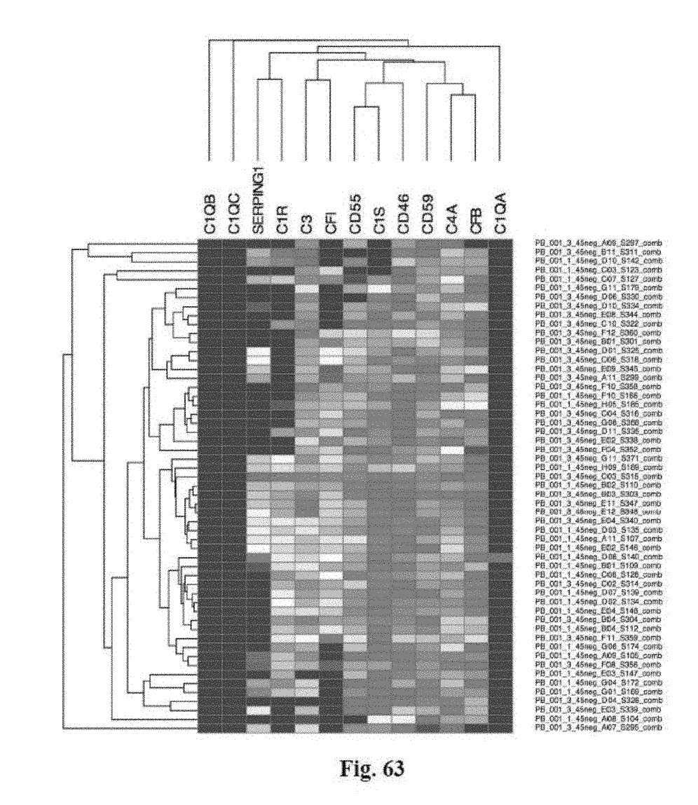

Finally, Applicants analyzed this subset of complement genes in CD45− cells confirmed through variable expression analysis to qualify as tumor-derived single cells (FIG. 63). Constituent expression of CD55/CD59/CD46 was observed. The complement “defense” genes (CD46, CD55 and CD59) are expressed quite uniformly across all six cell types previously analyzed herein and this is consistent with data in other tumor types analyzed. All of the tumor cells (55/55) express CD59, CD59 prevents C9 polymerization and thereby prevents attack complex formation. CIS is co-expressed with CD59 (microglia do not express CIS). CIS is required for activation of the classical pathway. There also exists a prominent subpopulation of tumor cells that express SERPING1, which inhibits CIS production. Genes differentially expressed in SERPING1(−) cells are enriched for upregulated genes in MCF7 cells (breast cancer cell line) during estradiol treatment for the primary tumor. The patient described herein was receiving hormone treatment therapy. This suggests that SERPING1 downregulation is a consequence of estradiol. SERPING1 is a C1 inhibitor. Not being bound by a theory, if SERPING1 is downregulated, it provides an explanation for CIS upregulation in these cells and provides an upstream target for deregulation of the complement system in these tumors.

Applicants also observed that the defense genes, CD46, CD55 and CD59, are correlated with a specific pattern of cell cycle. This pattern seems to be linked to a global pattern of whether malignant tumor cells express a “chromatin” or a “mitochondria” signature. Some tumors have higher levels of a large set of chromatin-related proteins, while the other tumors have lower chromatin-related gene expression and higher expression of oxidative phosphorylation and mitochondrial genes. This is a strong effect that exists within all tumor types. The link to the complement regulatory genes is that CD46 (and to a lower extent CD55) is highly correlated with the “chromatin” arm, which would suggest that despite their membrane-based function they are also linked to the chromatin, or to cell biology of the tumor. Not being bound by a theory, the defense proteins invoke a unique state in the cell to protect them, hence downregulation of the genes can provide a therapeutic effect by targeting more than complement activation.

Applicants also analyzed genes enriched in the complement pathway according to Gene Set Enrichment Analysis (GSEA) (Table 23). Not being bound by a theory, these genes may be used as biomarkers for activation of complement. Not being bound by a theory genes expressed on the cell surface may be used as biomarkers for determining an immune state of a tumor. The cell surface biomarkers may be used to stain tissue from a patient.

TABLE 23
Genes correlated with complement pathway in
each subset (Microglia, Tumor, and T cell)

| | | |
|---|---|---|
| Microglia | Tumor | T cell |

| | | | | | |
|---|---|---|---|---|---|
| 1 | LGALS9 | 1 | SLC9A3R1 | 1 | UQCRC1 |
| 2 | TNXB | 2 | CA5B | 2 | TCP1 |
| 3 | DBNL | 3 | POR | 3 | USP15 |
| 4 | PRDX1 | 4 | TMED10 | 4 | MED21 |
| 5 | SNX2 | 5 | MCFD2 | 5 | CHURC1 |
| 6 | SPCS1 | 6 | SLC7A11 | 6 | ZNF267 |
| 7 | EZR | 7 | PCED1B-AS1 | 7 | ERO1L |
| 8 | SAR1A | 8 | FAM73A | 8 | CARD16 |
| 9 | PPP1CA | 9 | DCXR | 9 | PIGB |
| 10 | ATP5O | 10 | PTP4A1 | 10 | RAB18 |
| 11 | PTPN3 | 11 | KPNA6 | 11 | CPSF3L |
| 12 | RHOG | | | 12 | CDK6 |
| 13 | SYNJ2 | | | 13 | GLUD1 |
| 14 | COPE | | | 14 | DPP3 |
| 15 | MTCH2 | | | 15 | PPP2R1A |
| 16 | PRDX6 | | | 16 | FKBP3 |
| 17 | SLC25A3 | | | 17 | PPP6R3 |
| 18 | PDIA6 | | | 18 | ERP29 |
| 19 | CYP4B1 | | | 19 | SNRPA1 |
| 20 | TPD52L2 | | | 20 | ARL6IP6 |
| 21 | CCT2 | | | 21 | CCNK |
| 22 | EDF1 | | | 22 | ATP6V1E1 |
| 23 | H2AFZ | | | 23 | SENP1 |
| 24 | STXBP2 | | | 24 | OAS3 |
| 25 | EIF4A1 | | | 25 | NXF1 |
| 26 | MOB1A | | | 26 | GID8 |
| | | | | 27 | NSA2 |
| | | | | 28 | SLC9A8 |
| | | | | 29 | BRCA1 |
| | | | | 30 | NADSYN1 |
| | | | | 31 | METTL23 |
| | | | | 32 | PLP2 |
| | | | | 33 | ZDHHC4 |
| | | | | 34 | ZFR |
| | | | | 35 | FAM96B |
| | | | | 36 | LAMTOR2 |
| | | | | 37 | EIF3A |
| | | | | 38 | XRCC5 |
| | | | | 39 | MGST3 |
| | | | | 40 | SKIV2L |
| | | | | 41 | NBEAL2 |
| | | | | 42 | PRDX4 |
| | | | | 43 | DNAJC1 |
| | | | | 44 | FAM105B |
| | | | | 45 | MLLT3 |
| | | | | 46 | GPN1 |
| | | | | 47 | IFI35 |
| | | | | 48 | ELOVL5 |
| | | | | 49 | STIP1 |
| | | | | 50 | GAPDH |
| | | | | 51 | EIF4G1 |

Genes are selected by having correlation of 0.5+ in at least:
50% of complement genes in microglia
50% of complement genes in tumor cells
80% of complement genes in T-cells

### Example 6

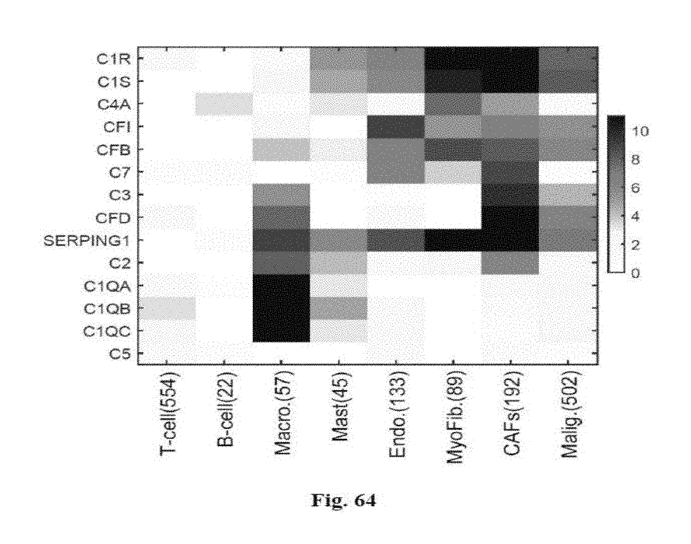

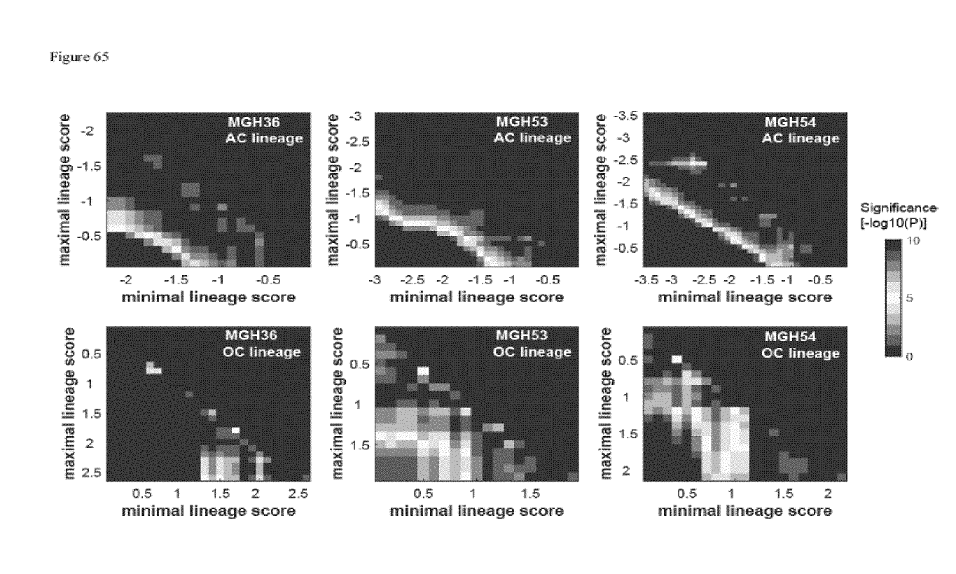

Applicants analyzed expression of complement genes by CAFs and macrophages in head and neck squamous cell carcinoma (HNSCC) (FIG. 64). 2150 single cells from 10 HNSCC tumors were profiled by single cell RNA-seq and were classified into 8 cells types based on tSNE analysis, as described herein for melanoma tumors. Shown are the average expression levels (log 2(TPM+1), of complement genes (Y-axis) in cells from each of the 8 cell types, demonstrating high expression of most complement genes by fibroblasts or macrophages. This observation is consistent with the patterns found in melanoma analysis. The predicted cell types (X-axis) are T-cells, B-cells, macrophages, mast cells, endothelial cells, myofibroblasts, CAFs, and malignant HNSCC cells; the number of cells classified to each cell type is indicated in parenthesis (X-axis). Consistent with the data from melanoma C1QA, B and C are highly expressed in macrophages. The analysis shows that expression signatures of complement genes is maintained across cancers. Not being bound by a theory, complement genes are a universal target for treating cancer. This result was previously not appreciated and unexpected because these signatures would not be detectable by sequencing of bulk tumors. Not being bound by a theory, analysis of tumors by single cell RNA-seq for the first time advantageously provides new targets for treating not only cancer, but any disease requiring a shift in an immune response.

The invention is further described by the following numbered paragraphs:

1. A method of diagnosing, prognosing and/or staging a condition or disorder having an immunological state, comprising detecting a first level of expression, activity and/or function of one or more signature genes or one or more products of one or more signature genes in one or more cell(s) of the disorder and comparing the detected level to a control level of signature gene or gene product expression, activity and/or function, wherein the one or more signature genes comprise a component of the complement system, and wherein a difference in the detected level and the control level indicates an immunologic state of the condition or disorder.

2. The method of numbered paragraph 1, wherein the one or more signature genes comprise C1S, C1R, C3, C4A, CFB, C1QA, C1QB, C1QC, CD46, CD55, CD59 or SERPING1.

3. The method of numbered paragraphs 1 or 2, wherein the immunologic state of the condition or disorder is characterized by the presence or absence of immune cells comprising myeloid-derived suppressor cells (MDSC), macrophages, dendritic cells (DC), natural killer cells (NK), T cells and/or B cells, wherein expression of the one or more signature genes correlates to the abundance of the immune cells.

4. The method of any one of numbered paragraphs 1 to 3, wherein the condition or disorder comprises autoimmune diseases, inflammatory diseases, infections or cancer.

5. The method of any one of numbered paragraphs 1 to 4, wherein the inflammatory disease comprises a pathogenic or non-pathogenic Th17 response.

6. The method of any one of numbered paragraphs 1 to 4, wherein the cancer comprises Non-Hodgkin's Lymphoma (NHL), clear cell Renal Cell Carcinoma (ccRCC), melanoma, sarcoma, leukemia or a cancer of the bladder, colon, brain, breast, head and neck, endometrium, lung, ovary, pancreas or prostate.

7. The method of numbered paragraph 6, wherein the cancer is a recurrent cancer.

8. The method of numbered paragraph 6, wherein the cancer is from a patient who progressed through chemotherapy.

9. The method of any one of numbered paragraphs 1 to 8, wherein the one or more signature genes comprises a gene that indicates the abundance of T cells.

10. The method of numbered paragraph 9, wherein the one or more signature genes is detected in CAFs.

11. The method of numbered paragraph 10, wherein the one or more signature genes comprises C1S, C1R, C3, C4A, CFB, or SERPING1.

12. The method of numbered paragraph 9, wherein the one or more signature genes is detected in macrophages.

13. The method of numbered paragraph 12, wherein the one or more signature genes comprises C1QA, C1QB or C1QC.

14. The method of any one of numbered paragraphs 1 to 8, wherein the one or more signature genes comprises a gene that indicates the abundance of B cells.

15. The method of numbered paragraph 14, wherein the one or more signature genes is detected in CAFs.

16. The method of numbered paragraph 15, wherein the one or more signature genes comprises C7 or C3.

17. The method of any one of numbered paragraphs 1 to 8, wherein the one or more signature genes comprises a gene that indicates the abundance of macrophages.

18. The method of numbered paragraph 17, wherein the one or more signature genes is detected in CAFs.

19. The method of numbered paragraph 18, wherein the one or more signature genes comprises C1S, C1R or CFB.

20. The method of any one of the preceding numbered paragraphs, wherein the level or expression of the one or more signature genes is determined by single-cell RNA sequencing.

21. The method of claim 20, wherein the single-cell RNA sequencing comprises single nucleus RNA-Seq.

22. The method of any one of the preceding numbered paragraphs, wherein level of expression, activity and/or function of one or more signature genes is determined by the level of expression of one or more products encoded by one or more signature genes in one or more cell(s).

23. The method of numbered paragraph 22, wherein the level of expression of one or more products encoded by one or more signature genes is determined by a colorimetric assay or absorbance assay.

24. The method of any one of the preceding numbered paragraphs, wherein level of expression, activity and/or function of one or more signature genes or one or more products of one or more signature genes in one or more cell(s) is determined by deconvolution of bulk expression data.

25. A method of treating or enhancing treatment of condition or disorder having an immunological state, which comprises administering an agent that increases or decreases the function, activity and/or expression of one or more signature genes or one or more products of one or more signature genes in one or more cell(s) of the condition or disorder, wherein the one or more signature genes comprise a component of the complement system, and wherein administering of the agent increases or decreases an immune response.

26. The method of numbered paragraph 25, wherein administering of the agent increases or decreases the abundance of an immune cell.

27. The method of numbered paragraph 26, wherein the agent increases or decreases the function, activity and/or expression of C1S, C1R, C3, C4A, CFB, C1QA, C1QB, C1QC, CD46, CD55, CD59, C5 or SERPING1(CFI).

28. The method of numbered paragraph 27, wherein the condition or disorder is cancer and the agent decreases the function, activity and/or expression CD46, CD55 or CD59, whereby malignant cells are susceptible to killing by complement activation.

29. The method of any of numbered paragraphs 25 to 28, wherein the agent comprises a CRISPR-Cas system that activates expression of the component of the complement system.

30. The method of any of numbered paragraphs 25 to 28, wherein the agent comprises a CRISPR-Cas system that targets the component of the complement system, whereby the component gene is knocked out or expression is decreased.

31. The method of any of numbered paragraphs 25 to 28, wherein the agent is an isolated natural product, whereby the component of the complement system is activated.

32. The method of numbered paragraph 31, wherein the agent comprises a metalloproteinase, whereby a component of the complement system is directly cleaved.

33. The method of numbered paragraph 31, wherein the agent comprises a serine protease, whereby a component of the complement system is directly cleaved.

34. The method of any of numbered paragraphs 25 to 28, wherein the agent comprises a therapeutic antibody or fragment thereof.

35. A method of treating cancer in a patient in need thereof comprising administering a therapeutically effective amount of an agent capable of targeting or binding to a component of the complement system presented on the surface of a cancer cell.

36. The method of numbered paragraph 35, wherein the component of the complement system is CD46, CD55 or CD59.

37. The method of numbered paragraph 36, wherein the agent is a therapeutic antibody or fragment thereof, antibody drug conjugate or fragment thereof, or a CAR T cell.

38. The method of numbered paragraph 35, wherein the cancer comprises Non-Hodgkin's Lymphoma (NHL), clear cell Renal Cell Carcinoma (ccRCC), melanoma, sarcoma, leukemia or a cancer of the bladder, colon, brain, breast, head and neck, endometrium, lung, ovary, pancreas or prostate.

39. A method of treating glioma, comprising administering to a subject in need thereof having glioma a therapeutically effective amount of an agent:
capable of reducing the expression or inhibiting the activity of one or more stem cell or progenitor cell signature genes or polypeptides; or
capable of targeting or binding to one or more cell surface exposed stem cell or progenitor cell signature polypeptides.

40. The method according to numbered paragraph 39, wherein said agent capable of targeting or binding to one or more cell surface exposed stem cell or progenitor cell signature polypeptides comprises a CAR T cell capable of targeting or binding to one or more cell surface exposed stem cell or progenitor cell signature polypeptides.

41. A method of treating glioma, comprising administering to a subject having glioma a therapeutically effective amount of an agent capable of inducing the expression or increasing the activity of one or more astrocyte and/or oligodendrocyte cell signature genes or polypeptides.

42. The method according to any of numbered paragraph 39 to 41, wherein said subject has not previously received chemotherapy and/or radiotherapy.

43. The method according to any of numbered paragraphs 39 to 42, comprising inducing differentiation of stem cells or progenitor cells comprised by the glioma.

44. The method according to numbered paragraph 43, wherein said differentiation comprises induction of expression or activity of one or more astrocyte and/or oligodendrocyte signature genes or polypeptides in the stem cells or progenitor cells.

45. The method according to any of numbered paragraphs 39 to 42, comprising reducing the viability of or rendering non-viable stem cells or progenitor cells comprised by the glioma.

46. A method of diagnosing, prognosing, or stratifying glioma, comprising determining expression or activity of one or more stem cell or progenitor cell signature genes or polypeptides in cells comprised by the glioma.

47. The method according to numbered paragraph 46, comprising determining the relative expression level of one or more stem cell or progenitor cell signature genes or polypeptides compared to one or more astrocyte and/or oligodendrocyte signature genes or polypeptides in the cells comprised by the glioma.

48. The method according to numbered paragraph 46 or 47, comprising determining the fraction of the cells comprised by the glioma, which express one or more stem cell or progenitor cell signature genes or polypeptides.

49. A method of identifying a therapeutic for glioma, comprising administering to a glioma cell in vitro a candidate therapeutic and monitoring expression or activity of one or more stem cell or progenitor cell signature genes or polypeptides.

50. The method according to numbered paragraph 49, wherein reduction in expression or activity of said one or more stem cell or progenitor cell signature genes or polypeptides is indicative of a therapeutic effect.

51. A method of monitoring glioma treatment or evaluating glioma treatment efficacy, comprising determining expression or activity of one or more stem cell or progenitor cell signature genes or polypeptides in cells comprised by the glioma.

52. The method according to numbered paragraph 51, comprising determining the relative expression level of one or more stem cell or progenitor cell signature genes or polypeptides compared to one or more astrocyte and/or oligodendrocyte signature genes or polypeptides in the cells comprised by the glioma.

53. The method according to numbered paragraph 51 or 52, comprising determining the fraction of the cells comprised by the glioma, which express one or more stem cell or progenitor cell signature genes or polypeptides.

54. A method of diagnosing, prognosing, or stratifying glioma, comprising identifying cells comprised by the glioma, which express one or more of CX3CR1, CD14, CD53, CD68, CD74, FCGR2A, HLA-DRA, or CSF1R. or one or more of MOBP, OPALIN, MBP, PLLP, CLDN11, MOG, or PLP1.

55. The method according to any of numbered paragraphs 39 to 54, wherein said stem cell or progenitor cell is a neural stem cell or progenitor cell.

56. The method according to any of numbered paragraphs 39 to 55, wherein said stem cell or progenitor cell signature genes or polypeptides are not oligodendrocyte precursor cell signature genes or polypeptides.

57. The method according to any of numbered paragraphs 39 to 56, wherein said glioma is oligodendroglioma.

58. The method according to any of numbered paragraphs 39 to 57, wherein said glioma is low grade glioma.

59. The method according to any of numbered paragraphs 39 to 58, wherein said glioma is grade II glioma.

60. The method according to any of numbered paragraphs 39 to 59, wherein said glioma is characterized by IDH1 and/or IDH2 mutations.

61. The method according to any of numbered paragraphs 39 to 60, wherein said glioma is characterized by CIC mutations.

62. The method according to any of numbered paragraphs 39 to 61, wherein said glioma is characterized by mutations in one or more gene selected from the group consisting of FAM120B, FGR1B, TP18, ESD, MTMR4, TUBB4A, H2AFV, EEF1B2, TMEM5, CEP170, EIF2AK2, SEC63, PTP4A1. RP11-556N21.1, ZEB2. DNAJC4, ZNF292, and ANKRD36.

63. The method according to any of numbered paragraphs 39 to 62, wherein said glioma is characterized by deletion of chromosome arms 1p and/or 19q.

64. The method according to any of numbered paragraphs 39 to 62, wherein said stem cell or progenitor cell signature gene is selected from SOX4, CCND2, SOX11, RBM6, HNRNPH1, HNRNPL, PTMA, TRA2A, SET, C6orf62, PTPRS, CHD7, CD24, H3F3B, C14orf23, NFIB, SRGAP2C, STMN2, SOX2, TFDP2, CORO1C. EIF4B, FBLIM1, SPDYE7P, TCF4, ORC6. SPDYE1, NCRUPAR. BAZ2B, NELL2, OPHN1. SPHKAP, RAB42, LOH12CR2, ASCL1, BOC, ZBTB8A, ZNF793, TOX3, EGFR, PGM5P2, EEF1A1, MALAT1, TATDN3, CCL5, EVI2A, LYZ, POU5F1, FBXO27, CAMK2N1, NEK5, PABPC1, AFMID, QPCTL, MBOAT1, HAPLN1. LOC90834, LRTOMT, GATM-AS1. AZGP1, RAMP2-AS1, SPDYE5. TNFAIP8L1.

65. The method according to any of numbered paragraphs 39 to 62, wherein said one or more stem cell or progenitor cell signature gene or polypeptide is selected from the group consisting of SOX4, SOX 11, SOX2, NFIB, ASCL1, CDH7, CD24, BOC, and TCF4.

66. The method according to any of numbered paragraphs 39 to 62, wherein said one or more stem cell or progenitor cell signature gene or polypeptide is selected from the group consisting of SOX4, CCND2, SOX 11, CDH7, CD24, NFIB, SOX2, TCF4, ASCL1, BOC, and EGFR.

67. The method according to any of numbered paragraphs 39 to 62, wherein said one or more stem cell or progenitor cell signature gene or polypeptide is selected from the group consisting of SOX 11, SOX4, NFIB TCF4, SOX2, CDH7, BOC, and CCND2.

68. The method according to any of numbered paragraphs 39 to 62, wherein said one or more stem cell or progenitor cell signature gene or polypeptide is selected from the group consisting of SOX 11, PTMA, NFIB, CCND2. SOX4, TCF4, CD24, CHD7, and SOX2.

69. The method according to any of numbered paragraphs 39 to 62, wherein said one or more stem cell or progenitor cell signature gene or polypeptide is selected from the group consisting of SOX2, SOX4, SOX11, MSI1, TERF2, CTNNB1, USP22, BRD3, CCND2, and PTEN.

70. The method according to any of numbered paragraphs 39 to 62, wherein said one or more stem cell or progenitor cell signature gene or polypeptide is selected from the SOX4, PTPRS, NFIB, CCND2, RBM6, SET, BAZ2B, TRA2A.

71. The method according to any of numbered paragraphs 39 to 62, wherein said stem cell or progenitor cell signature gene is selected from the group consisting of SOX2, SOX4, SOX6, SOX9. SOX11, CDH7, TCF4, BAZ2B, DCX, PDGFRA, DKK3, GABBR2, CA12, PLTP, IGFBP7, FABP7, LGR4, and ATP1A2.

72. The method according to any of numbered paragraphs 41 to 71, wherein said one or more astrocyte signature gene or polypeptide is selected from the group consisting of APOE, SPARCL1, SPOCK1, CRYAB, ALDOC, CLU, EZR. SORL1, MLC1, ABCA1, ATP1B2, PAPLN, CA12, BBOX1, RGMA, AGT, EEPD1, CST3, SSTR2, SOX9, RND3. EDNRB, GABRB1, PLTP, JUNB, DKK3, ID4, ADCYAP1R1, GLUL. EPAS1, PFKFB3. ANLN, HEPN1, CPE, RASL10A, SEMA6A. ZFP36L1, HEY1, PRLHR, TACR1, JUN, GADD45B, SLC1A3, CDC42EP4, MMD2, CPNE5, CPVL, RHOB, NTRK2, CBS, DOK5, TOB2, FOS, TRIL, NFKBIA, SLC1A2, MTHFD2, IER2, EFEMP1, ATP13A4, KCNIP2. ID1, TPCN1, LRRC8A, MT2A, FOSB, L1CAM, LLX1, HLA-E. PEA15, MT1X, 1L33, LPL, IGFBP7, C1 orf61, FXYD7, TIMP3. RASSF4, HNMT, JUND, NHSL1, ZFP36L2, SRPX, DTNA, ARHGEF26, SPON1, TBC1D10A, DGKG, LHFP, FTH1, NOG, LCAT, LRIG1, GATSL3. EGLN3, ACSL6, HEPACAM, ST6GAL2, KIF21A. SCG3, METTL7A, CHST9, RFX4, P2RY1, ZFAND5. TSPAN12, SLC39A11, NDRG2. HSPB8, IL11RA, SERPINA3, LYPD1, KCNH7, ATF3, TMEM151B, PSAP, HIF1A, PON2, HIF3A, MAFB, SCG2, GRIA1, ZFP36, GRAMD3, PER1, TNS1, BTG2, CASQ1, GPR75. TSC22D4, NRP1, DNASE2, DAND5, SF3A1, PRRT2, DNAJB1, F3; or selected from the group consisting of APOE, SPARCL1, ALDOC. CLU, EZR, SORL1, MLC1, ABCA1, ATP1B2. RGMA. AGT, EEPD1, CST3, SOX9, EDNRB, GABRB1, PLTP, JUNB, DKK3, ID4, ADCYAP1R1, GLUL, PFKFB3, CPE, ZFP36L1. JUN, SLC1A3, CDC42EP4, NTRK2, CBS, DOK5, FOS, TRIL, SLC1A2. ATP13A4. ID1, TPCN1, FOSB. LIX1, IL33, TIMP3, NHSL1, ZFP36L2, DTNA, ARHGEF26. TBC1D10A, LHFP, NOG, LCAT, LRIG1, GATSL3, ACSL6, HEPACAM, SCG3, RFX4, NDRG2, HSPB8, ATF3, PON2, ZFP36, PER1, BTG2, NRP1, PRRT2, F3; or selected from the group consisting of SPOCK1, CRYAB, PAPLN, CA12, BBOX1, SSTR2. RND3, EPAS1, ANLN, HEPN1, RASL10A, SEMA6A, HEY1, PRLHR, TACR1, GADD45B, MMD2, CPNE5, CPVL, RHOB, TOB2, NFKBIA, MTHFD2, IER2, EFEMP1. KCNIP2, LRRC8A, MT2A, L1CAM. HLA-E, PEA15, MT1X, LPL. IGFBP7, C1orf61, FXYD7, RASSF4, HNMT, JUND, SRPX. SPON1. DGKG. FTH1, EGLN3, ST6GAL2, KIF21A, METTL7A, CHST9, P2RY1, ZFAND5, TSPAN12, SLC39A11, IL11RA, SERPINA3, LYPD1, KCNH7, TMEM151B, PSAP. HIF1A, HIF3A, MAFB, SCG2, GRIA1, GRAMD3. TNS1, CASQ1, GPR75. TSC22D4, DNASE2, DAND5, SF3A1, DNAJB1.

73. The method according to any of numbered paragraphs 41 to 71, wherein said one or more oligodendrocyte signature gene or polypeptide is selected from the group consisting of LMF1, OLIG1. SNX22. POLR2F, LPPR1, GPR17, DLL3, ANGPTL2, SOX8, RPS2, FERMT1, PHLDA1. RPS23, NEU4, SLC1A1, LIMA1, ATCAY, SERINC5, CDH13, CXADR. LHFPL3, ARL4A. SHD, RPL31, GAP43. IFITM10, SIRT2. OMG. RGMB, HIPK2. APOD. NPPA, EEF1B2, RPS17L, FXYD6. MYT1, RGR, OLIG2, ZCCHC24. MTSS1, GNB2L1, C17orf76-AS1, ACTG1, EPN2, PGRMC1, TMSB10. NAP1L, EEF2, MIAT. CDHR1, TRAF4, TMEM97, NACA, RPSAP58, SCD, TNK2, RTKN, UQCRB, FA2H, MIF, TUBB3, COX7C, AMOTL2, THY1, NPM1, MARCKSL1, LIMS2, PHLDB1, RAB33A, GRIA2, OPCML, SHISA4, TMEFF2. ACAT2, HIP1, NME1, NXPH1. FDPS, MAP1A, DLL1, TAGLN3, PID1, KLRC2, AFAP1L2, LDHB, TUBB4A, ASIC1, TM7SF2, GRIA4, SGK1, P2RX7, WSCD1, ATP5E, ZDHHC9, MAML2, UGT8, C2orf27A, VIPR2, DHCR24, NME2, TCF12, MEST, CSPG4, GAS5, MAP2. LRRN1, GRIK2, FABP7, EIF3E, RPL13A, ZEB2. EIF3L, BIN1, FGFBP3, RAB2A. SNX1, KCNIP3. EBP, CRB1, RPS10-NUDT3, GPR37L1, CNP, DHCR7, MICAL1, TUBB, FAU, TMSB4X, PHACTR3; or selected from the group consisting of OLIG1, SNX22, GPR17, DLL3, SOX8, NEU4, SLC1A1, LIMA1, ATCAY, SERINC5, LHFPL3, SIRT2, OMG. APOD, MYT1, OLIG2, RTKN, FA2H, MARCKSL1, LIMS2, PHLDB1. RAB33A, OPCML, SHISA4, TMEFF2, NME1, NXPH1, GRIA4, SGK1, ZDHHC9, CSPG4, LRRN1, BIN1, EBP, CNP; or selected from the group consisting of LMF1, POLR2F, LPPR1, ANGPTL2, RPS2, FERMT1, PHLDA1, RPS23, CDH13, CXADR, ARL4A, SHD, RPL31, GAP43, IFITM10, RGMB, HIPK2, NPPA, EEF1B2, RPS17L, FXYD6, RGR, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, UQCRB, MIF, TUBB3, COX7C, AMOTL2, THY1, NPM1, GRIA2, ACAT2, HIP1, FDPS, MAP1A, DLL, TAGLN3, PID1, KLRC2, AFAP1L2, LDHB, TUBB4A, ASIC1, TM7SF2, P2RX7, WSCD1, ATP5E. MAML2, UGT8, C2orf27A. VIPR2, DHCR24, NME2. TCF12, MEST, GAS5, MAP2, GRIK2, FABP7, EIF3E, RPL13A, ZEB2. EIF3L, FGFBP3, RAB2A, SNX1. KCNIP3, CRB1, RPS10-NUDT3, GPR37L1, DHCR7, MICAL1, TUBB, FAU, TMSB4X, PHACTR3.

74. The method of any of numbered paragraphs 39 to 62, wherein the one or more signature genes is an indicator of a low-cycling or a high-cycling tumor.

75. The method of numbered paragraph 74, wherein the one or more signature genes comprises cyclin D3 (CCND3) or KDM5B (JAR1D1B), wherein CCND3 indicates high-cycling tumors and KDM5B indicates non-cycling cells.

76. An isolated cell characterized by comprising the expression of one or more a signature genes or polypeptides as defined in any of numbered paragraphs 64 to 73.

77. A glioma gene expression signature characterized by a signature gene or polypeptide as defined in any of numbered paragraphs 64 to 73.

78. A method of diagnosing, prognosing and/or staging a melanoma, comprising detecting a first level of expression, activity and/or function of one or more signature genes or one or more products of one or more signature genes in one or more cell(s) of the melanoma and comparing the detected level to a control level of signature gene or gene product expression, activity and/or function, wherein a difference in the detected level and the control level indicates a malignant, microenvironmental, or immunologic state of the melanoma.

79. The method of numbered paragraph 78, wherein the melanoma is a metastatic melanoma.

80. The method of any one of numbered paragraphs 78 to 79, wherein the melanoma is a recurrent melanoma.

81. The method of any one of numbered paragraphs 78 to 80, wherein the melanoma comprises a BRAF mutation.

82. The method of any one of numbered paragraphs 78 to 80, wherein the melanoma comprises an NRAS mutation.

83. The method of any one of numbered paragraphs 78 to 80, wherein the melanoma is from a patient who progressed through chemotherapy.

84. The method of numbered paragraph 83, wherein the chemotherapy is vemurafenib or a combination of vemurafenib and trametinib.

85. The method of any one of numbered paragraphs 78 to 84, wherein the one or more signature gene(s) is a MITF-high associated gene.

86. The method of any one of numbered paragraphs 78 to 84, wherein the one or more signature gene(s) is an AXL-high associated gene.

87. The method of any one of numbered paragraphs 78 to 84, wherein the one of more signature gene(s) comprises CXCL12 or CCL19.

88. The method of any one of numbered paragraphs 78 to 84, wherein the one of more signature gene(s) expresses PD-L2.

89. The method of any one of numbered paragraphs 78 to 84, wherein the one or more signature gene(s) comprises a gene that indicates the functional state of an immune cell from the tumor.

90. The method of numbered paragraph 89, wherein the one or more signature genes comprises a gene that indicates the abundance of T cells in the tumor.

91. The method of numbered paragraph 90, wherein the one or more signature genes comprises a signature gene of Table 15.

92. The method of numbered paragraph 90, wherein the one or more signature genes is detected in CAFs.

93. The method of numbered paragraph 92, wherein the one or more signature genes comprises CXCL2, CCL19, PD-L2, C1S, C1R, C3, C4A, CFB, HSD11B1, RARRES1, TME176A, TMEM176B or SERPING1.

94. The method of numbered paragraph 90, wherein the one or more signature genes is detected in macrophages.

95. The method of numbered paragraph 94, wherein the one or more signature genes comprises C1QA, C1QB or C1QC.

96. The method of numbered paragraph 90, wherein the one or more signature genes is detected in endothelial cells.

97. The method of numbered paragraph 96, wherein the one or more signature genes comprises PECAM1, LMO2, KIF19, IL3RA, RBP5. GP1BA, HAPLN3 or RSPO3.

98. The method of numbered paragraph 90, wherein the one or more signature genes is detected in melanoma cells.

99. The method of numbered paragraph 98, wherein the one or more signature genes comprises ceruloplasmin (CP).

100. The method of numbered paragraph 89, wherein the one or more signature genes comprises a gene that indicates the abundance of B cells in the tumor.

101. The method of numbered paragraph 100, wherein the one or more signature genes is detected in CAFs.

102. The method of numbered paragraph 101, wherein the one or more signature genes comprises CCL19, CLU, C7, KEL, C3, HSD11B1, RAI2, ABI3BP or CDX1.

103. The method of numbered paragraph 100, wherein the one or more signature genes is detected in endothelial cells.

104. The method of numbered paragraph 103, wherein the one or more signature genes comprises RBP5, ART4, GP1BA, or PKHD1L1.

105. The method of numbered paragraph 100, wherein the one or more signature genes is detected in melanoma cells.

106. The method of numbered paragraph 105, wherein the one or more signature genes comprises ceruloplasmin (CP).

107. The method of numbered paragraph 89, wherein the one or more signature genes comprises a gene that indicates the abundance of macrophages in the tumor.

108. The method of numbered paragraph 107, wherein the one or more signature genes is detected in CAFs.

109. The method of numbered paragraph 108, wherein the one or more signature genes comprises C1S, C1R, CFB or HSD11B1.

110. The method of numbered paragraph 107, wherein the one or more signature genes is detected in endothelial cells.

111. The method of numbered paragraph 110, wherein the one or more signature genes comprises PECAM1, LMO2, or IL3RA.

112. The method of numbered paragraph 107, wherein the one or more signature genes is detected in melanoma cells.

113. The method of numbered paragraph 112, wherein the one or more signature genes comprises ceruloplasmin (CP).

114. The method of numbered paragraph 89, wherein the one or more signature genes comprises a gene that indicates the functional state of a T cell from the tumor.

115. The method of numbered paragraph 114, wherein the T cell comprises a Treg cell.

116. The method of numbered paragraph 115, wherein the one or more signature genes comprises a signature gene of Table 12.

117. The method of numbered paragraph 116, wherein the one or more signature genes comprises FOXP3 or IL2RA.

118. The method of numbered paragraph 89, wherein the one or more signature genes comprises a gene that indicates the exhaustion state of an immune cell of the tumor.

119. The method of numbered paragraph 118, wherein the one or more signature genes comprises a signature gene of Table 13, or Table 14.

120. The method of numbered paragraph 119, wherein the one or more signature genes comprises PDCD1, TIGIT, HAVCR2, SIT1, LAG3, CTLA4, FAM3C, TNFRSF9, SYT11, GUSBP3, SIRPG, LY6E, CXCL13, SUMO2. IL2RG, CD74, CBLB, FOXN3, SLA, FKBP1A, CD27, SP100, IK, CCL3, CXCL13, TNFRSF1B, RGS2, RNF19A, INPP5F, XCL2, HLA-DMA, UQCRC1, WARS, EIF3L, KCNK5. TMBIM6, CD200, ZC3H7A, SH2D1A, ATP1B3, MYO7A, THADA, PARK7, EGR2, FDFT1, CRTAM, IFI16, LAG3, NFATC1, TIM3, PD-1, BTLA or CBLB.

121. The method of any one of numbered paragraphs 78 to 84, wherein the one or more signature genes comprises a signature gene that indicates cell cycle state.

122. The method of numbered paragraph 121, wherein the one or more signature genes is an indicator of a low-cycling or a high-cycling tumor.

123. The method of numbered paragraph 122, wherein the one or more signature genes comprises cyclin D3 (CCND3) or KDM5B (JAR1D1B), wherein CCND3 indicates high-cycling tumors and KDM5B indicates non-cycling cells.

124. The method of any one of numbered paragraphs 78 to 84, wherein the one or more signature gene(s) comprises a complement system gene.

125. The method of numbered paragraph 124, wherein the one or more signature genes comprises C1S, C1R, C3, C4A, CFB or SERPING1.

126. The method of any one of numbered paragraphs 78 to 84, wherein the one or more signature genes comprises a signature gene that is an indication of drug resistance.

127. The method of any one of numbered paragraphs 78 to 126, wherein the level or expression of the one or more signature genes is determined by single-cell RNA sequencing.

128. The method of any one of numbered paragraphs 78 to 127, wherein level of expression, activity and/or function of one or more signature genes is determined by the level of expression of one or more products encoded by one or more signature genes in one or more cell(s) of the melanoma.

129. The method of numbered paragraph 128, wherein the level of expression of one or more products encoded by one or more signature genes is determined by a colorimetric assay or absorbance assay.

130. The method of any one of numbered paragraphs 78 to 129, wherein level of expression, activity and/or function of one or more signature genes or one or more products of one or more signature genes in one or more cell(s) of the melanoma is determined by deconvolution of the bulk expression properties of a tumor.

131. A method for monitoring a subject undergoing a treatment or therapy for a melanoma comprising detecting a level of expression, activity and/or function of one or more signature genes or one or more products of one or more signature genes of the melanoma in the absence of the treatment or therapy and comparing the level of expression, activity and/or function of one or more signature genes or one or more products of one or more signature genes in the presence of the treatment or therapy, wherein a difference in the level of expression, activity and/or function of one or more signature genes or one or more products of one or more signature genes in the presence of the treatment or therapy indicates whether the patient is responsive to the treatment or therapy.

132. The method of numbered paragraph 131, wherein the treatment or therapy modulates expression of one or more signature genes that indicates the functional state of an immune cell from the tumor.

133. The method of numbered paragraph 131, wherein the treatment or therapy modulates expression of one or more signature genes that indicates cell cycle state.

134. A method of treating melanoma or enhancing treatment of a melanoma, which comprises administering an agent that increases the function of one or more signature genes or one or more products of one or more signature genes in one or more cell(s) of the melanoma, wherein the one or more signature genes or one or more products of one or more signature genes comprises a signature gene corresponding to abundance of an immune cell.

135. The method of numbered paragraph 134, wherein the one or more signature genes comprises a gene that indicates the abundance of T cells in the tumor.

136. The method of numbered paragraph 135, wherein the one or more signature genes comprises a signature gene of Table 15.

137. The method of numbered paragraph 135, wherein the one or more signature genes is detected in CAFs.

138. The method of numbered paragraph 137, wherein the one or more signature genes comprises CXCL12, CCL9. PD-L2, C1S, C1R, C3, C4A, CFB, HSD11B1, RARRES1, TMEM176A, TMEM176B or SERPING1.

139. The method of numbered paragraph 135, wherein the one or more signature genes is detected in macrophages.

140. The method of numbered paragraph 139, wherein the one or more signature genes comprises C1QA, C1QB or C1QC.

141. The method of numbered paragraph 135, wherein the one or more signature genes is detected in endothelial cells.

142. The method of numbered paragraph 141, wherein the one or more signature genes comprises PECAM1, LMO2, KIF19, IL3RA, RBP5, GP1BA, HAPLN3 or RSPO3.

143. The method of numbered paragraph 135, wherein the one or more signature genes is detected in melanoma cells.

144. The method of numbered paragraph 143, wherein the one or more signature genes comprises ceruloplasmin (CP).

145. The method of numbered paragraph 134, wherein the one or more signature genes comprises a gene that indicates the abundance of B cells in the tumor.

146. The method of numbered paragraph 145, wherein the one or more signature genes is detected in CAFs.

147. The method of numbered paragraph 146, wherein the one or more signature genes comprises CCL19, CLU, C7, KEL, C3, HSD11B1, RAI2, ABI3BP or CDX1.

148. The method of numbered paragraph 145, wherein the one or more signature genes is detected in endothelial cells.

149. The method of numbered paragraph 148, wherein the one or more signature genes comprises RBP5. ART4, GP1BA, or PKHD1L1.

150. The method of numbered paragraph 145, wherein the one or more signature genes is detected in melanoma cells.

151. The method of numbered paragraph 150, wherein the one or more signature genes comprises ceruloplasmin (CP).

152. The method of numbered paragraph 134, wherein the one or more signature genes comprises a gene that indicates the abundance of macrophages in the tumor.

153. The method of numbered paragraph 152, wherein the one or more signature genes is detected in CAFs.

154. The method of numbered paragraph 153, wherein the one or more signature genes comprises C1S, C1R, CFB or HSD11B1.

155. The method of numbered paragraph 152, wherein the one or more signature genes is detected in endothelial cells.

156. The method of numbered paragraph 155, wherein the one or more signature genes comprises PECAM1, LMO2, or IL3RA.

157. The method of numbered paragraph 152, wherein the one or more signature genes is detected in melanoma cells.

158. The method of numbered paragraph 157, wherein the one or more signature genes comprises ceruloplasmin (CP). The method of numbered paragraph 138, wherein the one or more signature genes comprises CXCL12 or CCL19.

159. A method of treating melanoma or enhancing treatment of a melanoma, which comprises administering an agent that increases the function of one or more signature genes or one or more products of one or more signature genes in one or more cell(s) of the melanoma, wherein the one or more signature genes or one or more products of one or more signature genes comprises a signature gene of Table 12.

160. A method of treating melanoma or enhancing treatment of a melanoma, which comprises administering an agent that decreases the function of one or more signature genes or one or more products of one or more signature genes in one or more cell(s) of the melanoma, wherein the one or more signature genes or one or more products of one or more signature genes comprises a signature gene of Table 13, or Table 14.

161. The method of numbered paragraph 160, wherein the one or more signature genes comprises PDCD1, TIGIT. HAVCR2, SIT1, LAG3, CTLA4, FAM3C, TNFRSF9, SYT11, GUSBP3. SIRPG, LY6E, CXCL13, SUMO2, IL2RG, CD74, CBLB, FOXN3, SLA, FKBP1A, CD27, SP00, IK, CCL3, CXCL13, TNFRSF1B, RGS2, RNF19A, INPP5F. XCL2, HLA-DMA, UQCRC1, WARS, EIF3L, KCNK5, TMBIM6, CD200 ZC3H7A, SH2D1A, A7P1B3, MYO7A, THADA, PARK7. EGR2. FDFT1, CRTAM, IFI16, LAG3, NFATC1, TIM3, PD-1, BTLA or CBLB.

162. The method of numbered paragraph 161, wherein the agent inhibits SIT1. SIRPG, or CBLB.

163. A method of treating melanoma or enhancing treatment of a melanoma, which comprises administering an agent that modulates the activity and/or expression of one or more signature genes or one or more products of one or more signature genes in one or more cell(s) of the melanoma, wherein the one or more signature genes or one or more products of one or more signature genes is a complement system gene or gene product.

164. The method of numbered paragraph 163, wherein the agent enhances the activity and/or expression of C1S, C1R, C3, C4A, CFB, C1QA, C1QB, or C1QC.

165. The method of numbered paragraph 164, wherein the agent comprises a CRISPR-Cas system that activates expression of a complement system gene.

166. The method of numbered paragraph 163, wherein the agent targets a complement defense gene selected from the group consisting of CD46, CD55, and CD59.

167. The method of numbered paragraph 166, wherein the agent comprises a CRISPR-Cas system that targets the complement defense gene, whereby the gene is knocked out or expression is decreased.

168. The method of numbered paragraph 163, wherein the agent is a natural product, whereby the complement system is activated in a tumor.

169. The method of numbered paragraph 168, wherein the agent comprises a metalloproteinase, whereby complement system components are directly cleaved in a tumor.

170. The method of numbered paragraph 168, wherein the agent comprises a serine protease, whereby complement system components are directly cleaved in a tumor.

171. A method of identifying at least one tumor specific T Cell receptor (TCR) for use in adoptive cell transfer, said method comprising:
(a) identifying by sequencing, TCRs from single tumor infiltrating T cells obtained from a tumor sample;
(b) selecting the TCRs that are clonal and/or are derived from a T cell that expresses one or more signature genes of exhaustion; and
(c) cloning the selected TCRs into a non-naturally occurring vector.

172. The method of numbered paragraph 171, wherein the one or more signature genes of exhaustion comprises PDCD1, TIGIT, HAVCR2, SIT1, LAG3, CTLA4, FAM3C, TNFRSF9, SYT11, GUSBP3, SIRPG, LY6E, CXCL13, SUMO2, IL2RG, CD74, CBLB, FOXN3, SLA, FKBP1A, CD27, SP100, IK, CCL3, CXCL13, TNFRSF1B, RGS2, RNF19A, INPP5F, XCL2, HLA-DMA, UQCRC1, WARS, EIF3L, KCNK5, TMBIM6, CD200, ZC3H7A, SH2D1A, ATP1B3. MYO7A. THADA, PARK7, EGR2. FDFT1, CRTAM, IFII6, LAG3, NFATC1, TIM3, PD-1, BTLA or CBLB.

173. A method of treating a subject in need thereof suffering from cancer comprising administering at least one activated T cell to the subject expressing at least one TCR pair identified by the method according to numbered paragraph 171.

174. A non-naturally occurring T cell expressing a tumor specific TCR pair identified by the method according to numbered paragraph 171.

175. A personalized cancer treatment for a patient in need thereof comprising: (a) determining clonality of TCRs in tumor infiltrating T cells from the patient, and/or
(b) detecting expression of one or more signature genes for exhaustion, and/or
(c) detecting expression of one or more signature genes correlated to T cell abundance; and
(d) administering an agent that stimulates the patients preexisting immune response if (i) at least one clonal TCR is determined and/or (ii) one or more signature genes for exhaustion is detected and/or (iii) one or more signature genes correlated to T cell abundance is detected.

176. The personalized cancer treatment of numbered paragraph 175, wherein the clonality and/or expression of one or more signature genes is detected by single cell RNA sequencing.

177. The method of numbered paragraph 176, wherein the single-cell RNA sequencing comprises single nucleus RNA-Seq.

178. The personalized cancer treatment of numbered paragraph 175, wherein the agent is a checkpoint inhibitor.

### REFERENCES

1. D. Hanahan, R. A. Weinberg, Hallmarks of cancer: the next generation. Cell. 144, 646-674 (2011).
2, C. E. Meacham, S. J. Morrison, Tumour heterogeneity and cancer cell plasticity. Nature. 501, 328-337 (2013).
3. F. S. Hodi et al., Improved Survival with Ipilimumab in Patients with Metastatic Melanoma. N. Engl. J. Med. 363, 711-723 (2010).
4. J. R. Brahmer et al., Phase I study of single-agent anti-programmed death-1 (MDX-1106) in refractory solid tumors: safety, clinical activity, pharmacodynamics, and immunologic correlates. J. Clin. Oncol. Off. J. Am. Soc. Clin. Oncol. 28, 3167-3175 (2010).
5. J. R. Brahmer et al., Safety and Activity of Anti-PD-L1 Antibody in Patients with Advanced Cancer. N. Engl. J. Med. 366, 2455-2465 (2012).
6. S. L. Topalian et al., Safety, activity, and immune correlates of anti-PD-1 antibody in cancer. N. Engl. J. Med. 366, 2443-2454 (2012).
7. O. Hamid et al., Safety and tumor responses with lambrolizumab (anti-PD-1) in melanoma. N. Engl. J. Med. 369, 134-144 (2013).
8. J. S. Weber et al., Safety, efficacy, and biomarkers of nivolumab with vaccine in ipilimumabrefractory or -naïve melanoma. J. Clin. Oncol. Off J. Am. Soc. Clin. Oncol. 31, 4311-4318 (2013).
9. K. M. Mahoney, M. B. Atkins, Prognostic and predictive markers for the new immunotherapies. Oncol. Williston Park N. 28 Suppl 3, 39-48 (2014).
10. J. Larkin et al., Combined Nivolumab and Ipilimumab or Monotherapy in Untreated Melanoma. N. Engl. J. Med. 373, 23-34 (2015).
11. A. Snyder et al., Genetic basis for clinical response to CTLA-4 blockade in melanoma. N. Engl. J Med. 371, 2189-2199 (2014).
12. N. Wagle et al., Dissecting Therapeutic Resistance to RAF Inhibition in Melanoma by Tumor Genomic Profiling. J. Clin. Oncol. (2011), doi: 10.1200/JCO.2010.33.2312.
13. E. M. Van Allen et al., The genetic landscape of clinical resistance to RAF inhibition in metastatic melanoma. Cancer Discov. 4, 94-109 (2014).
14. A. K. Shalek et al., Single-cell transcriptomics reveals bimodality in expression and splicing in immune cells. Nature. 498, 236-240 (2013).
15. A. P. Patel et al., Single-cell RNA-seq highlights intratumoral heterogeneity in primary glioblastoma. Science. 344, 1396-1401 (2014).
16. E. Z. Macosko et al., Highly Parallel Genome-wide Expression Profiling of Individual Cells Using Nanoliter Droplets. Cell. 161, 1202-1214 (2015).
17. L. van der Maaten, G. Hinton, Visualizing Data using t-SNE. 9, 2579-2605 (2008).
18. M. Ester, H. Kriegel, J. Sander, and X. Xu. “A density-based algorithm for discovering clusters in large spatial databases with noise,” in Proc. 2nd Int. Conf. Knowledge Discovery and Data Mining (KDD'96), 1996, pp. 226-231.
19. M. L. Whitfield, L. K. George, G. D. Grant, C. M. Perou, Common markers of proliferation. Nat. Rev. Cancer. 6, 99-106 (2006).
20. A. Roesch et al, A temporarily distinct subpopulation of slow-cycling melanoma cells is required for continuous tumor growth. Cell. 141, 583-594 (2010).
21. A first-in-human phase I study of the CDK4/6 inhibitor, LY2835219, for patients with advanced cancer. J. Clin. Oncol. (available at meetinglibrary.asco.org/content/111069-132).
22, C. M. Johannessen et al., A melanocyte lineage program confers resistance to MAP kinase pathway inhibition. Nature. 504, 138-142 (2013).
23. D. J. Konieczkowski et al., A melanoma cell state distinction influences sensitivity to MAPK pathway inhibitors. Cancer Discov. 4, 816-827 (2014).
24. L. A. Garraway et al., Integrative genomic analyses identify MITF as a lineage survival oncogene amplified in malignant melanoma. Nature. 436, 117-122 (2005).
25. Z. Zhang et al., Activation of the AXL kinase causes resistance to EGFR-targeted therapy in lung cancer. Nat. Genet. 44, 852-860 (2012).
26. X. Wu et al., AXL kinase as a novel target for cancer therapy. Oncotarget. 5, 9546-9563 (2014).
27. A. D. Boiko et al., Human melanoma-initiating cells express neural crest nerve growth factor receptor CD271. Nature. 466, 133-137 (2010).
28. K. S. Hoek et al., In vivo Switching of Human Melanoma Cells between Proliferative and Invasive States. Cancer Res. 68, 650-656 (2008).
29. J. Müller et al., Low MITF/AXL ratio predicts early resistance to multiple targeted drugs in melanoma. Nat. Commun. 5, 5712 (2014).
30. F. Z. Li, A. S. Dhillon, R. L. Anderson, G. McArthur, P. T. Ferrao, Phenotype switching in melanoma: implications for progression and therapy. Mol. Cell. Oncol. 5, 31 (2015).
31. W. Hugo et al., Non-genomic and Immune Evolution of Melanoma Acquiring MAPKi Resistance. Cell. 162, 1271-1285 (2015).
32. R. Nazarian et al., Melanomas acquire resistance to B-RAF(V600E) inhibition by RTK or N-RAS upregulation. Nature. 468, 973-977 (2010).
33. J. Barretina et al., The Cancer Cell Line Encyclopedia enables predictive modelling of anticancer drug sensitivity. Nature. 483, 603-607 (2012).
34. W. H. Fridman, F. Pagès, C. Sautes-Fridman, J. Galon, The immune contexture in human tumours: impact on clinical outcome. Nat. Rev. Cancer. 12, 298-306 (2012).
35. S. L. Carter et al., Absolute quantification of somatic DNA alterations in human cancer. Nat. Biotechnol. 30, 413-421 (2012).
36. Roadmap Epigenomics Consortium et al., Integrative analysis of 111 reference human epigenomes. Nature. 518, 317-330 (2015).
37. R. Akbani et al., Genomic Classification of Cutaneous Melanoma. Cell. 161, 1681-1696 (2015).
38. M. M. Markiewski et al., Modulation of the antitumor immune response by complement. Nat. Immunol. 9, 1225-1235 (2008).
39. E. J. Wherry, T cell exhaustion. Nat. Immunol. 12, 492-499 (2011).
40. L. Chen, D. B. Flies, Molecular mechanisms of T cell co-stimulation and co-inhibition. Nat. Rev. Immunol. 13, 227-242 (2013).
41. H. Borghaei et al., Nivolumab versus Docetaxel in Advanced Nonsquamous Non-Small-Cell Lung Cancer. N. Engl. J. Med. 373, 1627-1639 (2015).
42. R. J. Motzer et al., Nivolumab versus Everolimus in Advanced Renal-Cell Carcinoma. N. Engl. J. Med. 373, 1803-1813 (2015).
43. N. A. Rizvi et al., Cancer immunology. Mutational landscape determines sensitivity to PD-1 blockade in non-small cell lung cancer. Science. 348, 124-128 (2015).
44. E. M. Van Allen et al., Genomic correlates of response to CTLA-4 blockade in metastatic melanoma. Science. 350, 207-211 (2015).
45. E. J. Wherry et al., Molecular signature of CD8+ T cell exhaustion during chronic viral infection. Immunity. 27, 670-684 (2007).
46. L. Baitsch et al., Exhaustion of tumor-specific CD8+ T cells in metastases from melanoma patients. J. Clin. Invest. 121, 2350-2360 (2011).
47. G. J. Martinez et al., The transcription factor NFAT promotes exhaustion of activated CD8+ T cells. Immunity. 42, 265-278 (2015).
48. S. D. Blackburn, H. Shin, G. J. Freeman, E. J. Wherry, Selective expansion of a subset of exhausted CD8 T cells by αPD-L1 blockade. Proc. Natl. Acad. Sci. U.S.A (2008) (available at agris.fao.org/agris-search/search.do?recordID=US201301547699).
49. L. Baitsch et al., Extended Co-Expression of Inhibitory Receptors by Human CD8 T-Cells Depending on Differentiation. Antigen-Specificity and Anatomical Localization. PLoS ONE. 7, e30852 (2012).
50. S. Picelli et al., Smart-seq2 for sensitive full-length transcriptome profiling in single cells. Nat. Methods. 10, 1096-1098 (2013).
51. J. J. Trombetta et al., Preparation of Single-Cell RNA-Seq Libraries for Next Generation Sequencing. Curr. Protoc. Mol. Biol. Ed. Frederick M Ausubel Al. 107, 4.22.1-4.22.17 (2014).
52. H. Li, R. Durbin, Fast and accurate short read alignment with Burrows-Wheeler transform. Bioinforma. Oxf. Engl. 25, 1754-1760 (2009).
53. A. McKenna et al., The Genome Analysis Toolkit: a MapReduce framework for analyzing next generation DNA sequencing data. Genome Res. 20, 1297-1303 (2010).
54. M. F. Berger et al., The genomic complexity of primary human prostate cancer. Nature. 470, 214-20 (2011).
55. K. Cibulskis et al., Sensitive detection of somatic point mutations in impure and heterogeneous cancer samples. Nat. Biotechnol. 31, 213-9 (2013).
56, C. T. Saunders et al., Strelka: accurate somatic small-variant calling from sequenced tumornormal sample pairs. Bioinforma. Oxf. Engl. 28, 1811-7 (2012).
57. A. H. Ramos et al., Oncotator: cancer variant annotation tool. Hum. Mutat. 36, E2423-9 (2015).
58. E. S. Venkatraman, A. B. Olshen, A faster circular binary segmentation algorithm for the analysis of array CGH data. Bioinforma. Oxf Engl. 23, 657-63 (2007).
59. B. Langmead, C. Trapnell, M. Pop, S. L. Salzberg, Ultrafast and memory-efficient alignment of short DNA sequences to the human genome. Genome Biol. 10, R25 (2009).
60. B. Li, C. N. Dewey, RSEM: accurate transcript quantification from RNA-Seq data with or without a reference genome. BMC Bioinformatics. 12, 323 (2011).
61. A. K. Shalek et al., Single-cell RNA-seq reveals dynamic paracrine control of cellular variation. Nature. 510, 363-369 (2014).
62. M. L. Whitfield et al., Identification of genes periodically expressed in the human cell cycle and their expression in tumors. Mol. Biol. Cell. 13, 1977-2000 (2002).
63. D. E. Campton et al., High-recovery visual identification and single-cell retrieval of circulating tumor cells for genomic analysis using a dual-technology platform integrated with automated immunofluorescence staining. BMC Cancer. 15, 360 (2015).
64. I. Skaland et al., Comparing subjective and digital image analysis HER2/neu expression scores with conventional and modified FISH scores in breast cancer. J. Clin. Pathol. 61, 68-71(2008).
65. J. Konsti et al., Development and evaluation of a virtual microscopy application for automated assessment of Ki-67 expression in breast cancer. BMC Clin. Pathol. 11, 3 (2011).
66. W. Hugo et al., Non-genomic and Immune Evolution of Melanoma Acquiring MAPKi Resistance. Cell. 162, 1271-1285 (2015).
67. L. Baitsch et al., Extended Co-Expression of Inhibitory Receptors by Human CD8 T-Cells Depending on Differentiation, Antigen-Specificity and Anatomical Localization. PLoS ONE. 7, e30852 (2012).
68. E. J. Wherry et al., Molecular signature of CD8+ T cell exhaustion during chronic viral infection. Immunity. 27, 670-684 (2007).
69. G. J. Martinez et al., The transcription factor NFAT promotes exhaustion of activated CD8+ T cells. Immunity. 42, 265-278 (2015).
70. E. A. Eisenhauer et al., New response evaluation criteria in solid tumours: revised RECIST guideline (version 1.1). Eur. J. Cancer Oxf. Engl. 1990. 45, 228-247 (2009).
71. J. Barretina et al., The Cancer Cell Line Encyclopedia enables predictive modelling of anticancer drug sensitivity. Nature. 483, 603-607 (2012).
72. Kreso, A. & Dick, J. E. Evolution of the cancer stem cell model. Cell stem cell 14, 275-291, (2014).
73. Baylin, S. B. & Jones, P. A. A decade of exploring the cancer epigenome—biological and translational implications. Nature reviews. Cancer 11, 726-734, (2011).
74. Suva, M. L., Riggi, N. & Bernstein, B. E. Epigenetic reprogramming in cancer. Science 339, 1567-1570. (2013).
75. Bao. S., Wu, Q., McLendon, R. E., Hao, Y., Shi, Q., Hjelmeland, A. B. et al. Glioma stem cells promote radioresistance by preferential activation of the DNA damage response. Nature 444, 756-760, (2006).
76, Chen, J., Li, Y., Yu, T. S., McKay, R. M., Burns, D. K., Kemie, S. G. et al. A restricted cell population propagates glioblastoma growth after chemotherapy. Nature 488, 522-526. (2012).
77. Ito, K., Bemardi, R., Morotti, A., Matsuoka, S., Saglio, G., Ikeda, Y. et al. PML targeting eradicates quiescent leukaemia-initiating cells. Nature 453, 1072-1078, (2008).
78. Lathia, J. D., Gallagher, J., Heddleston, J. M., Wang, J., Eyler, C. E., Macswords, J. et al. Integrin alpha 6 regulates glioblastoma stem cells. Cell stem cell 6, 421-432, (2010).
79. Piccirillo, S. G., Reynolds, B. A., Zanetti, N., Lamorte, G., Binda. E., Broggi, G. et al. Bone morphogenetic proteins inhibit the tumorigenic potential of human brain tumour-initiating cells. Nature 444, 761-765, (2006).
80. Singh, S. K., Hawkins, C., Clarke, I. D., Squire, J. A., Bayani, J., Hide, T. et al. Identification of human brain tumour initiating cells. Nature 432, 396-401, (2004).
81. Anido, J., Saez-Borderias, A., Gonzalez-Junca. A., Rodon, L., Folch, G., Carmona, M. A. et al. TGF-beta Receptor Inhibitors Target the CD44(high)/Id1(high) Glioma-Initiating Cell Population in Human Glioblastoma. Cancer cell 18, 655-668, (2010).
82. Son, M. J., Woolard, K., Nam, D. H., Lee, J. & Fine, H. A. SSEA-1 is an enrichment marker for tumor-initiating cells in human glioblastoma. Cell stem cell 4, 440-452, (2009).
83. Srikanth, M., Kim, J., Das, S. & Kessler, J. A. BMP signaling induces astrocytic differentiation of clinically derived oligodendroglioma propagating cells. Mol Cancer Res 12 283-294 (2014).
84. Friedmann-Morvinski, D., Bushong, E. A., Ke. E., Soda, Y., Marumoto, T., Singer, O. et al. Dedifferentiation of neurons and astrocytes by oncogenes can induce gliomas in mice. Science 338, 1080-1084, (2012).
85. Dalerba, P., Kalisky, T., Sahoo, D., Rajendran. P. S., Rothenberg, M. E., Leyrat, A. A. et al. Single-cell dissection of transcriptional heterogeneity in human colon tumors. Nature biotechnology 29 1120-1127 (2011).
86. Lawson, D. A., Bhakta, N. R., Kessenbrock, K, Prummel. K. D., Yu, Y., Takai, K. et al. Single-cell analysis reveals a stem-cell program in human metastatic breast cancer cells. Nature 526 131-135 (2015).
87. Jaitin, D. A., Kenigsberg, E., Keren-Shaul, H., Elefant, N., Paul, F., Zaretsky, I. et al. Massively parallel single-cell RNA-seq for marker-free decomposition of tissues into cell types. Science 343 776-779 (2014).
88. Pollen, A. A., Nowakowski, T. J., Shuga. J., Wang. X., Leyrat, A. A., Lui, J. H. et al. Low-coverage single-cell mRNA sequencing reveals cellular heterogeneity and activated signaling pathways in developing cerebral cortex. Nature biotechnology 32 1053-1058 (2014).
89. Treutlein, B., Brownfield, D. G., Wu, A. R., Neff, N. F., Mantalas, G. L., Espinoza, F. H. et al. Reconstructing lineage hierarchies of the distal lung epithelium using single-cell RNA-seq. Nature 509 371-375 (2014).
90. Zeisel, A., Munoz-Manchado, A. B., Codeluppi, S., Lonnerberg, P., La Manno, G., Jureus, A. et al. Brain structure. Cell types in the mouse cortex and hippocampus revealed by single-cell RNA-seq. Science 347 1138-1142 (2015).
91. Suva, M. L. & Louis, D. N. Next-generation molecular genetics of brain tumours. Current opinion in neurology 26, 681-687, (2013).
92. Louis, D. N., Perry, A., Burger, P., Ellison, D. W., Reifenberger, G., von Deimling, A. et al. International Society Of Neuropathology—Haarlem consensus guidelines for nervous system tumor classification and grading. Brain pathology 24, 429-435, (2014).
93. Picelli, S., Faridani, O. R., Bjorklund, A. K., Winberg, G., Sagasser, S. & Sandberg, R. Full-length RNA-seq from single cells using Smart-seq2. Nat Protoc 9 171-181 (2014).
94. Butovsky, O., Jedrychowski, M. P., Moore, C. S., Cialic, R., Lanser, A. J., Gabriely, G. et al. Identification of a unique TGF-beta-dependent molecular and functional signature in microglia. Nat Neurosci 17 131-143 (2014).
95. Rousseau, A., Nutt, C. L., Betensky, R. A., Iafrate, A. J., Han, M., Ligon, K. L. et al. Expression of oligodendroglial and astrocytic lineage markers in diffuse gliomas: use of YKL-96. ApoE, ASCL1, and NKX2-2. Journal of neuropathology and experimental neurology 65 1149-1156 (2006).
97. Zhang, Y., Chen, K., Sloan, S. A., Bennett, M. L., Scholze, A. R., O'Keeffe, S. et al. An RNA-sequencing transcriptome and splicing database of glia, neurons, and vascular cells of the cerebral cortex. J Neurosci 34 11929-11947 (2014).
98. Louis, D. N., Ohgaki, H., Wiestler. O. D., Cavenee, W. K., Burger, P. C., Jouvet, A. et al. The 2007 WHO classification of tumours of the central nervous system. Acta neuropathologica 114, 97-109, (2007).
99. Feng. W., Khan, M. A., Bellvis, P., Zhu, Z., Bernhardt, O., Herold-Mende. C. et al. The chromatin remodeler CHD7 regulates adult neurogenesis via activation of SoxC transcription factors. Cell stem cell 13, 62-72, (2013).
100. Ikushima H., Todo, T., Ino, Y., Takahashi, M., Miyazawa, K. & Miyazono, K. Autocrine TGF-beta signaling maintains tumorigenicity of glioma-initiating cells through Sry-related HMG-box factors. Cell stem cell 5, 504-514, (2009).
101. Suva, M. L., Rheinbay, E., Gillespie, S. M., Patel, A. P., Wakimoto, H., Rabkin. S. D. et al. Reconstructing and reprogramming the tumor-propagating potential of glioblastoma stem-like cells. Cell 157, 580-594, (2014).
102. Mille, F., Tamayo-Orrego, L., Levesque, M., Remke, M., Korshunov, A., Cardin, J. et al. The Shh receptor Boc promotes progression of early medulloblastoma to advanced tumors. Developmental cell 31, 34-47, (2014).
103. Panchision, D. M., Chen, H. L., Pistollato, F., Papini, D., Ni, H. T. & Hawley, T. S. Optimized flow cytometric analysis of central nervous system tissue reveals novel functional relationships among cells expressing CD133, CD15, and CD24. Stem cells 25 1560-1570 (2007).
104. Rheinbay, E., Suva, M. L., Gillespie, S. M., Wakimoto, H., Patel, A. P., Shahid, M. et al. An Aberrant Transcription Factor Network Essential for Wnt Signaling and Stem Cell Maintenance in Glioblastoma. Cell reports 3, 1567-1579, (2013).
105. Miller, J. A., Ding. S. L., Sunkin, S. M., Smith, K. A., Ng, L., Szafer, A. et al. Transcriptional landscape of the prenatal human brain. Nature 508, 199-206, (2014).
106. Darmanis, S., Sloan, S. A., Zhang, Y., Enge, M., Caneda. C., Shuer, L. M. et al. A survey of human brain transcriptome diversity at the single cell level. Proceedings of the National Academy of Sciences of the United States of America, (2015).
107. Kelly, J. J., Blough, M. D., Stechishin, O. D., Chan, J. A., Beauchamp, D., Perizzolo, M. et al. Oligodendroglioma cell lines containing t(1;19)(q10;p10). Neuro-oncology 12 745-755 (2010).
108. Sugiarto, S., Persson, A. I., Munoz, E. G., Waldhuber, M., Lamagna, C., Andor, N. et al. Asymmetry-defective oligodendrocyte progenitors are glioma precursors. Cancer cell 20 328-340 (2011).
109. Aguirre, A., Dupree, J. L., Mangin, J. M. & Gallo, V. A functional role for EGFR signaling in myelination and remyelination. Nat Neurosci 10 990-1002 (2007).
110. Shah, N. M., Marchionni, M. A., Isaacs, 1., Stroobant, P. & Anderson, D. J. Glial growth factor restricts mammalian neural crest stem cells to a glial fate. Cell 77 349-360 (1994).
111. Shin, J., Berg, D. A., Zhu, Y., Shin, J. Y., Song, J., Bonaguidi, M. A. et al. Single-Cell RNA-Seq with Waterfall Reveals Molecular Cascades underlying Adult Neurogenesis. Cell stem cell 17, 360-372, (2015).
112, Cancer Genome Atlas Research, N., Brat, D. J., Verhaak, R. G., Aldape, K. D., Yung, W. K., Salama, S. R. et al. Comprehensive, Integrative Genomic Analysis of Diffuse Lower-Grade Gliomas. The New England journal of medicine 372, 2481-2498, (2015).
113. Lange, C. & Calegari, F. Cdks and cyclins link G1 length and differentiation of embryonic, neural and hematopoietic stem cells. Cell Cycle 9 1893-1900 (2010).
114. Koyama-Nasu, R, Nasu-Nishimura, Y., Todo, T., Ino, Y., Saito, N., Aburatani, H. et al. The critical role of cyclin D2 in cell cycle progression and tumorigenicity of glioblastoma stem cells. Oncogene 32 3840-3845 (2013).
115. Bettegowda, C., Agrawal, N., Jiao, Y., Sausen, M., Wood, L. D., Hruban, R. H. et al. Mutations in CIC and FUBP1 contribute to human oligodendroglioma. Science 333 1453-1455 (2011).
116. Padul, V., Epari, S., Moiyadi, A., Shetty, P. & Shirsat, N. V. ETV/Pea3 family transcription factor-encoding genes are overexpressed in CIC-mutant oligodendrogliomas. Genes, chromosomes & cancer 54, 725-733, (2015).
117. Liu, C., Sage, J. C., Miller, M. R, Verhaak, R. G., Hippenmeyer, S., Vogel, H. et al. Mosaic analysis with double markers reveals tumor cell of origin in glioma. Cell 146 209-221 (2011).
118. Ducray, F. & Idbaih, A. Neuro-oncology: anaplastic oligodendrogliomas-value of early chemotherapy. Nat Rev Neurol 9 7-8 (2013).
119. Satija, R., Farrell, J. A., Gennert, D., Schier, A. F. & Regev, A. Spatial reconstruction of single-cell gene expression data. Nature biotechnology 33 495-502 (2015).
120. Mohapatra, G., Betensky, R. A., Miller, E. R., Carey, B., Gaumont, L. D., Engler, D. A. et al. Glioma test array for use with formalin-fixed, paraffin-embedded tissue: array comparative genomic hybridization correlates with loss of heterozygosity and fluorescence in situ hybridization. J Mol Diagn 8 268-276 (2006).
121, Cibulskis, K., McKenna. A., Fennell, T., Banks. E., DePristo, M. & Getz, G. ContEst: estimating cross-contamination of human samples in next-generation sequencing data. Bioinformatics 27 2601-2602 (2011).
122, Costello, M., Pugh, T. J., Fennell, T. J., Stewart, C., Lichtenstein, L., Meldrim, J. C. et al. Discovery and characterization of artifactual mutations in deep coverage targeted capture sequencing data due to oxidative DNA damage during sample preparation. Nucleic Acids Res 41 e67 (2013).
123. Zhang, Y., Sloan, S. A., Clarke, L. E., Caneda. C., Plaza, C. A., Blumenthal, P. D. et al. Purification and Characterization of Progenitor and Mature Human Astrocytes Reveals Transcriptional and Functional Differences with Mouse. Neuron 89, 37-53, (2016).
124. Kowalczyk. M. S., Tirosh, I., Heckl, D., Rao, T. N., Dixit, A., Haas, B. J. et al. Single-cell RNA-seq reveals changes in cell cycle and differentiation programs upon aging of hematopoietic stem cells. Genome Res 25; 1860-1872 (2015).

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method of diagnosing, prognosing and/or staging a condition or disorder having an immunological state, comprising detecting a first level of expression, activity and/or function of one or more signature genes or one or more products of one or more signature genes in one or more cell(s) of the disorder and comparing the detected level to a control level of signature gene or gene product expression, activity and/or function, wherein the one or more signature genes comprise a component of the complement system, and wherein a difference in the detected level and the control level indicates an immunologic state of the condition or disorder.

2. The method of claim 1, wherein the one or more signature genes comprise C1S, C1R, C3, C4A, CFB, C1QA, C1QB, C1QC, CD46, CD55, CD59 or SERPING1.

3. The method of claim 1, wherein the immunologic state of the condition or disorder is characterized by the presence or absence of immune cells comprising myeloid-derived suppressor cells (MDSC), macrophages, dendritic cells (DC), natural killer cells (NK), T cells and/or B cells, wherein expression of the one or more signature genes correlates to the abundance of the immune cells.

4. The method of claim 1, wherein the condition or disorder comprises autoimmune diseases, inflammatory diseases, infections or cancer.

5. The method of claim 1, wherein the inflammatory disease comprises a pathogenic or non-pathogenic Th17 response.

6. The method of claim 1, wherein the cancer comprises Non-Hodgkin's Lymphoma (NHL), clear cell Renal Cell Carcinoma (ccRCC), melanoma, sarcoma, leukemia or a cancer of the bladder, colon, brain, breast, head and neck, endometrium, lung, ovary, pancreas or prostate.

7. The method of claim 6, wherein the cancer is a recurrent cancer.

8. The method of claim 6, wherein the cancer is from a patient who progressed through chemotherapy.

9. The method of claim 1, wherein the one or more signature genes comprises a gene that indicates the abundance of T cells.

10. The method of claim 9, wherein the one or more signature genes is detected in CAFs.

11. The method of claim 10, wherein the one or more signature genes comprises C1S, C1R, C3, C4A, CFB, or SERPING1.

12. The method of claim 9, wherein the one or more signature genes is detected in macrophages.

13. The method of claim 12, wherein the one or more signature genes comprises C1QA, C1QB or C1QC.

14. The method of claim 1, wherein the one or more signature genes comprises a gene that indicates the abundance of B cells.

15. The method of claim 14, wherein the one or more signature genes is detected in CAFs.

16. The method of claim 15, wherein the one or more signature genes comprises C7 or C3.

17. The method of claim 1, wherein the one or more signature genes comprises a gene that indicates the abundance of macrophages.

18. The method of claim 17, wherein the one or more signature genes is detected in CAFs.

19. The method of claim 18, wherein the one or more signature genes comprises C1S, C1R or CFB.

20. The method of claim 1, wherein the level or expression of the one or more signature genes is determined by single-cell RNA sequencing.

21. The method of claim 20, wherein the single-cell RNA sequencing comprises single nucleus RNA-Seq.

22. The method of claim 1, wherein level of expression, activity and/or function of one or more signature genes is determined by the level of expression of one or more products encoded by one or more signature genes in one or more cell(s).

23. The method of claim 22, wherein the level of expression of one or more products encoded by one or more signature genes is determined by a colorimetric assay or absorbance assay.

24. The method of claim 1, wherein level of expression, activity and/or function of one or more signature genes or one or more products of one or more signature genes in one or more cell(s) is determined by deconvolution of bulk expression data.

25. A method of treating or enhancing treatment of condition or disorder having an immunological state, which comprises administering an agent that increases or decreases the function, activity and/or expression of one or more signature genes or one or more products of one or more signature genes in one or more cell(s) of the condition or disorder, wherein the one or more signature genes comprise a component of the complement system, and wherein administering of the agent increases or decreases an immune response.

26. The method of claim 25, wherein administering of the agent increases or decreases the abundance of an immune cell.

27. The method of claim 26, wherein the agent increases or decreases the function, activity and/or expression of C1S, C1R, C3, C4A, CFB, C1QA, C1QB, C1QC, CD46, CD55, CD59, C5 or SERPING1(CFI).

28. The method of claim 27, wherein the condition or disorder is cancer and the agent decreases the function, activity and/or expression CD46, CD55 or CD59, whereby malignant cells are susceptible to killing by complement activation.

29. The method of claim 25, wherein the agent comprises a CRISPR-Cas system that activates expression of the component of the complement system.

30. The method of claim 25, wherein the agent comprises a CRISPR-Cas system that targets the component of the complement system, whereby the component gene is knocked out or expression is decreased.

31. The method of claim 25, wherein the agent is an isolated natural product, whereby the component of the complement system is activated.

32. The method of claim 31, wherein the agent comprises a metalloproteinase, whereby a component of the complement system is directly cleaved.

33. The method of claim 31, wherein the agent comprises a serine protease, whereby a component of the complement system is directly cleaved.

34. The method of claim 25, wherein the agent comprises a therapeutic antibody or fragment thereof.

35. A method of treating cancer in a patient in need thereof comprising administering a therapeutically effective amount of an agent capable of targeting or binding to a component of the complement system presented on the surface of a cancer cell.

36. The method of claim 35, wherein the component of the complement system is CD46, CD55 or CD59.

37. The method of claim 36, wherein the agent is a therapeutic antibody or fragment thereof, antibody drug conjugate or fragment thereof, or a CAR T cell.

38. The method of claim 35, wherein the cancer comprises Non-Hodgkin's Lymphoma (NHL), clear cell Renal Cell Carcinoma (ccRCC), melanoma, sarcoma, leukemia or a cancer of the bladder, colon, brain, breast, head and neck, endometrium, lung, ovary, pancreas or prostate.

39. A method of treating glioma, comprising administering to a subject in need thereof having glioma a therapeutically effective amount of an agent:
capable of reducing the expression or inhibiting the activity of one or more stem cell or progenitor cell signature genes or polypeptides; or
capable of targeting or binding to one or more cell surface exposed stem cell or progenitor cell signature polypeptides.

40. The method according to claim 39, wherein said agent capable of targeting or binding to one or more cell surface exposed stem cell or progenitor cell signature polypeptides comprises a CAR T cell capable of targeting or binding to one or more cell surface exposed stem cell or progenitor cell signature polypeptides.

41. A method of treating glioma, comprising administering to a subject having glioma a therapeutically effective amount of an agent capable of inducing the expression or increasing the activity of one or more astrocyte and/or oligodendrocyte cell signature genes or polypeptides.

42. The method according to claim 41, wherein said subject has not previously received chemotherapy and/or radiotherapy.

43. The method according to claim 42, comprising inducing differentiation of stem cells or progenitor cells comprised by the glioma.

44. The method according to claim 43, wherein said differentiation comprises induction of expression or activity of one or more astrocyte and/or oligodendrocyte signature genes or polypeptides in the stem cells or progenitor cells.

45. The method according to claim 41, comprising reducing the viability of or rendering non-viable stem cells or progenitor cells comprised by the glioma.

46. A method of diagnosing, prognosing, or stratifying glioma, comprising determining expression or activity of one or more stem cell or progenitor cell signature genes or polypeptides in cells comprised by the glioma.

47. The method according to claim 46, comprising determining the relative expression level of one or more stem cell or progenitor cell signature genes or polypeptides compared to one or more astrocyte and/or oligodendrocyte signature genes or polypeptides in the cells comprised by the glioma.

48. The method according to claim 46, comprising determining the fraction of the cells comprised by the glioma, which express one or more stem cell or progenitor cell signature genes or polypeptides.

49. A method of identifying a therapeutic for glioma, comprising administering to a glioma cell in vitro a candidate therapeutic and monitoring expression or activity of one or more stem cell or progenitor cell signature genes or polypeptides.

50. The method according to claim 49, wherein reduction in expression or activity of said one or more stem cell or progenitor cell signature genes or polypeptides is indicative of a therapeutic effect.

51. A method of monitoring glioma treatment or evaluating glioma treatment efficacy, comprising determining expression or activity of one or more stem cell or progenitor cell signature genes or polypeptides in cells comprised by the glioma.

52. The method according to claim 51, comprising determining the relative expression level of one or more stem cell or progenitor cell signature genes or polypeptides compared to one or more astrocyte and/or oligodendrocyte signature genes or polypeptides in the cells comprised by the glioma.

53. The method according to claim 51, comprising determining the fraction of the cells comprised by the glioma, which express one or more stem cell or progenitor cell signature genes or polypeptides.

54. A method of diagnosing, prognosing, or stratifying glioma, comprising identifying cells comprised by the glioma, which express one or more of CX3CR1, CD14, CD53, CD68, CD74, FCGR2A, HLA-DRA, or CSF1R, or one or more of MOBP, OPALIN, MBP, PLLP, CLDN11, MOG, or PLP1.

55. The method according to claim 54, wherein said stem cell or progenitor cell is a neural stem cell or progenitor cell.

56. The method according to claim 55, wherein said stem cell or progenitor cell signature genes or polypeptides are not oligodendrocyte precursor cell signature genes or polypeptides.

57. The method according to claim 56, wherein said glioma is oligodendroglioma.

58. The method according to claim 57, wherein said glioma is low grade glioma.

59. The method according to claim 58, wherein said glioma is grade II glioma.

60. The method according to claim 39, wherein said glioma is characterized by IDH1 and/or IDH2 mutations.

61. The method according to claim 39, wherein said glioma is characterized by CIC mutations.

62. The method according to claim 39, wherein said glioma is characterized by mutations in one or more gene selected from the group consisting of FAM120B, FGR1B, TP18, ESD, MTMR4, TUBB4A, H2AFV, EEF1B2, TMEM5, CEP170, EIF2AK2, SEC63, PTP4A1, RP11-556N21.1, ZEB2, DNAJC4, ZNF292, and ANKRD36.

63. The method according to claim 39, wherein said glioma is characterized by deletion of chromosome arms 1p and/or 19q.

64. The method according to claim 39, wherein said stem cell or progenitor cell signature gene is selected from SOX4, CCND2, SOX11, RBM6, HNRNPH1, HNRNPL, PTMA, TRA2A, SET, C6orf62, PTPRS, CHD7, CD24, H3F3B, C14orf23, NFIB, SRGAP2C, STMN2, SOX2, TFDP2, CORO1C, EIF4B, FBLIM1, SPDYE7P, TCF4, ORC6, SPDYE1, NCRUPAR, BAZ2B, NELL2, OPHN1, SPHKAP, RAB42, LOH12CR2, ASCL1, BOC, ZBTB8A, ZNF793, TOX3, EGFR, PGM5P2, EEF1A1, MALAT1, TATDN3, CCL5, EVI2A, LYZ, POU5F1, FBXO27, CAMK2N1, NEK5, PABPC1, AFMID, QPCTL, MBOAT1, HAPLN1, LOC90834, LRTOMT, GATM-AS1, AZGP1, RAMP2-AS1, SPDYE5, TNFAIP8L1.

65. The method according to claim 39, wherein said one or more stem cell or progenitor cell signature gene or polypeptide is selected from the group consisting of SOX4, SOX11, SOX2, NFIB, ASCL1, CDH7, CD24, BOC, and TCF4.

66. The method according to claim 39, wherein said one or more stem cell or progenitor cell signature gene or polypeptide is selected from the group consisting of SOX4, CCND2, SOX11, CDH7, CD24, NFIB, SOX2, TCF4, ASCL1, BOC, and EGFR.

67. The method according to claim 39, wherein said one or more stem cell or progenitor cell signature gene or polypeptide is selected from the group consisting of SOX11, SOX4, NFIB TCF4, SOX2, CDH7, BOC, and CCND2.

68. The method according to claim 39, wherein said one or more stem cell or progenitor cell signature gene or polypeptide is selected from the group consisting of SOX11, PTMA, NFIB, CCND2, SOX4, TCF4, CD24, CHD7, and SOX2.

69. The method according to claim 39, wherein said one or more stem cell or progenitor cell signature gene or polypeptide is selected from the group consisting of SOX2, SOX4, SOX11, MSI1, TERF2, CTNNB1, USP22, BRD3, CCND2, and PTEN.

70. The method according to claim 39, wherein said one or more stem cell or progenitor cell signature gene or polypeptide is selected from the SOX4, PTPRS, NFIB, CCND2, RBM6, SET, BAZ2B, TRA2A.

71. The method according to claim 39, wherein said stem cell or progenitor cell signature gene is selected from the group consisting of SOX2, SOX4, SOX6, SOX9, SOX11, CDH7, TCF4, BAZ2B, DCX, PDGFRA, DKK3, GABBR2, CA12, PLTP, IGFBP7, FABP7, LGR4, and ATP1A2.

72. The method according to claim 41, wherein said one or more astrocyte signature gene or polypeptide is selected from the group consisting of APOE, SPARCL1, SPOCK1, CRYAB, ALDOC, CLU, EZR SORL1, MLC1, ABCA1, ATP1B2, PAPLN, CA12, BBOX1, RGMA, AGT, EEPD1, CST3, SSTR2, SOX9, RND3, EDNRB, GABRB1, PLTP, JUNB, DKK3, ID4, ADCYAP1R1, GLUL, EPAS1, PFKFB3, ANLN, HEPN1, CPE, RASL10A, SEMA6A, ZFP36L1, HEY1, PRLHR, TACR1, JUN, GADD45B, SLC1A3, CDC42EP4, MMD2, CPNE5, CPVL, RHOB, NTRK2, CBS, DOK5, TOB2, FOS, TRIL, NFKBIA, SLC1A2, MTHFD2, IER2, EFEMP1, ATP13A4, KCNIP2, ID1, TPCN1, LRRC8A, MT2A, FOSB, L1CAM, LIX1, HLA-E, PEA15, MT1X, 1L33, LPL, IGFBP7, C1orf61, FXYD7, TIMP3, RASSF4, HNMT, JUND, NHSL1, ZFP36L2, SRPX, DTNA, ARHGEF26, SPON1, TBC1D10A, DGKG, LHFP, FTH1, NOG, LCAT, LRIG1, GATSL3, EGLN3, ACSL6, HEPACAM, ST6GAL2, KIF21A, SCG3, METTL7A, CHST9, RFX4, P2RY1, ZFAND5, TSPAN12, SLC39A11, NDRG2, HSPB8, IL11RA, SERPINA3, LYPD1, KCNH7, ATF3, TMEM151B, PSAP, HIF1A, PON2, HIF3A, MAFB, SCG2, GRIA1, ZFP36, GRAMD3, PER1, TNS1, BTG2, CASQ1, GPR75, TSC22D4, NRP1, DNASE2, DAND5, SF3A1, PRRT2, DNAJB1, F3; or selected from the group consisting of APOE, SPARCL1, ALDOC, CLU, EZR, SORL1, MLC1, ABCA1, ATP1B2, RGMA, AGT, EEPD1, CST3, SOX9, EDNRB, GABRB1, PLTP, JUNB, DKK3, ID4, ADCYAP1R1, GLUL, PFKFB3, CPE, ZFP36L1, JUN, SLC1A3, CDC42EP4, NTRK2, CBS, DOK5, FOS, TRIL, SLC1A2, ATP13A4, ID1, TPCN1, FOSB, LIX1, 1L33, TIMP3, NHSL1, ZFP36L2, DTNA, ARHGEF26, TBC1D10A, LHFP, NOG, LCAT, LRIG1, GATSL3, ACSL6, HEPACAM, SCG3, RFX4, NDRG2, HSPB8, ATF3, PON2, ZFP36, PER1, BTG2, NRP1, PRRT2, F3: or selected from the group consisting of SPOCK1, CRYAB, PAPLN, CA12, BBOX1, SSTR2, RND3, EPAS1, ANLN, HEPN1, RASL10A, SEMA6A, HEY1, PRLHR, TACR1, GADD45B, MMD2, CPNE5, CPVL, RHOB, TOB2, NFKBIA, MTHFD2, IER2, EFEMP1, KCNIP2, LRRC8A, MT2A, L1CAM, HLA-E, PEA15, MT1X, LPL, IGFBP7, C1orf61, FXYD7, RASSF4, HNMT, JUND, SRPX, SPON1, DGKG, FTH1, EGLN3, ST6GAL2, KIF21A, METTL7A, CHST9, P2RY1, ZFAND5, TSPAN12, SLC39A11, IL11RA, SERPINA3, LYPD1, KCNH7, TMEM151B, PSAP, HIF1A, HIF3A, MAFB, SCG2, GRIA1, GRAMD3, TNS1, CASQ1, GPR75, TSC22D4, DNASE2, DAND5, SF3A1, DNAJB1.

73. The method according to claim 41, wherein said one or more oligodendrocyte signature gene or polypeptide is selected from the group consisting of LMF1, OLIG1, SNX22, POLR2F, LPPR1, GPR17, DLL3, ANGPTL2, SOX8, RPS2, FERMT1, PHLDA1, RPS23, NEU4, SLC1A1, LIMA1, ATCAY, SERINC5, CDH13, CXADR, LHFPL3, ARL4A, SHD, RPL31, GAP43, IFITM10, SIRT2, OMG, RGMB, HIPK2, APOD, NPPA, EEF1B2, RPS17L, FXYD6, MYT1, RGR, OLIG2, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, RTKN, UQCRB, FA2H, MIF, TUBB3, COX7C, AMOTL2, THY1, NPM1, MARCKSL1, LIMS2, PHLDB1, RAB33A, GRIA2, OPCML, SHISA4, TMEFF2, ACAT2, HIP1, NME1, NXPH1, FDPS, MAP1A, DLL1, TAGLN3, PID1, KLRC2, AFAP1L2, LDHB, TUBB4A, ASIC1, TM7SF2, GRIA4, SGK1, P2RX7, WSCD1, ATP5E, ZDHHC9, MAML2, UGT8, C2orf27A, VIPR2, DHCR24, NME2, TCF12, MEST, CSPG4, GAS5, MAP2, LRRN1, GRIK2, FABP7, EIF3E, RPL13A, ZEB2, EIF3L, BIN1, FGFBP3, RAB2A, SNX1, KCNIP3, EBP, CRB1, RPS10-NUDT3, GPR37L1, CNP, DHCR7, MICAL1, TUBB, FAU, TMSB4X, PHACTR3; or selected from the group consisting of OLIG1, SNX22, GPR17, DLL3, SOX8, NEU4, SLC1A1, LIMA1, ATCAY, SERINC5, LHFPL3, SIRT2, OMG, APOD, MYT1, OLIG2, RTKN, FA2H, MARCKSL1, LIMS2, PHLDB1, RAB33A, OPCML, SHISA4, TMEFF2, NME1, NXPH1, GRIA4, SGK1, ZDHHC9, CSPG4, LRRN1, BIN1, EBP, CNP; or selected from the group consisting of LMF1, POLR2F, LPPR1, ANGPTL2, RPS2, FERMT1, PHLDA1, RPS23, CDH13, CXADR, ARL4A, SHD, RPL31, GAP43, IFITM10, RGMB, HIPK2, NPPA, EEF1B2, RPS17L, FXYD6, RGR, ZCCHC24, MTSS1, GNB2L1, C17orf76-AS1, ACTG1, EPN2, PGRMC1, TMSB10, NAP1L1, EEF2, MIAT, CDHR1, TRAF4, TMEM97, NACA, RPSAP58, SCD, TNK2, UQCRB, MIF, TUBB3, COX7C, AMOTL2, THY1, NPM1, GRIA2, ACAT2, HIP1, FDPS, MAP1A, DLL, TAGLN3, PID1, KLRC2, AFAP1L2, LDHB, TUBB4A, ASIC1, TM7SF2, P2RX7, WSCD1, ATP5E, MAML2, UGT8, C2orf27A, VIPR2, DHCR24, NME2, TCF12, MEST, GAS5, MAP2, GRIK2, FABP7, EIF3E, RPL13A, ZEB2, EIF3L, FGFBP3, RAB2A, SNX1, KCNIP3, CRB1, RPS10-NUDT3, GPR37L1, DHCR7, MICAL1, TUBB, FAU, TMSB4X, PHACTR3.

74. The method of claim 39, wherein the one or more signature genes is an indicator of a low-cycling or a high-cycling tumor.

75. The method of claim 74, wherein the one or more signature genes comprises cyclin D3 (CCND3) or KDM5B (JAR1D1B), wherein CCND3 indicates high-cycling tumors and KDM5B indicates non-cycling cells.

76. An isolated cell characterized by comprising the expression of one or more a signature genes or polypeptides as defined in claim 64.

77. A glioma gene expression signature characterized by a signature gene or polypeptide as defined in claim 64.

78. A method of diagnosing, prognosing and/or staging a melanoma, comprising detecting a first level of expression, activity and/or function of one or more signature genes or one or more products of one or more signature genes in one or more cell(s) of the melanoma and comparing the detected level to a control level of signature gene or gene product expression, activity and/or function, wherein a difference in the detected level and the control level indicates a malignant, microenvironmental, or immunologic state of the melanoma.

79. The method of claim 78, wherein the melanoma is a metastatic melanoma.

80. The method of claim 78, wherein the melanoma is a recurrent melanoma.

81. The method of claim 78, wherein the melanoma comprises a BRAF mutation.

82. The method of claim 78, wherein the melanoma comprises an NRAS mutation.

83. The method of claim 78, wherein the melanoma is from a patient who progressed through chemotherapy.

84. The method of claim 83, wherein the chemotherapy is vemurafenib or a combination of vemurafenib and trametinib.

85. The method of claim 78, wherein the one or more signature gene(s) is a MITF-high associated gene.

86. The method of claim 78, wherein the one or more signature gene(s) is an AXL-high associated gene.

87. The method of claim 78, wherein the one of more signature gene(s) comprises CXCL12 or CCL19.

88. The method of claim 78, wherein the one of more signature gene(s) expresses PD-L2.

89. The method of claim 78, wherein the one or more signature gene(s) comprises a gene that indicates the functional state of an immune cell from the tumor.

90. The method of claim 89, wherein the one or more signature genes comprises a gene that indicates the abundance of T cells in the tumor.

91. The method of claim 90, wherein the one or more signature genes comprises a signature gene of Table 15.

92. The method of claim 90, wherein the one or more signature genes is detected in CAFs.

93. The method of claim 92, wherein the one or more signature genes comprises CXCL12, CCL19, PD-L2, C1S, C1R, C3, C4A, CFB, HSD11B1, RARRES1, TMFAM176A, TMEM176B or SERPING1.

94. The method of claim 90, wherein the one or more signature genes is detected in macrophages.

95. The method of claim 94, wherein the one or more signature genes comprises C1QA, C1QB or C1QC.

96. The method of claim 90, wherein the one or more signature genes is detected in endothelial cells.

97. The method of claim 96, wherein the one or more signature genes comprises PECAM1, LMO2, KIF19, IL3RA, RBP5, GP1BA, HAPLN3 or RSPO3.

98. The method of claim 90, wherein the one or more signature genes is detected in melanoma cells.

99. The method of claim 98, wherein the one or more signature genes comprises ceruloplasmin (CP).

100. The method of claim 89, wherein the one or more signature genes comprises a gene that indicates the abundance of B cells in the tumor.

101. The method of claim 100, wherein the one or more signature genes is detected in CAFs.

102. The method of claim 101, wherein the one or more signature genes comprises CCL19, CLU, C7, KEL, C3, HSD11B1, RAI2, ABI3BP or CDX1.

103. The method of claim 100, wherein the one or more signature genes is detected in endothelial cells.

104. The method of claim 103, wherein the one or more signature genes comprises RBP5, ART4, GP1BA, or PKHD1L1.

105. The method of claim 100, wherein the one or more signature genes is detected in melanoma cells.

106. The method of claim 105, wherein the one or more signature genes comprises ceruloplasmin (CP).

107. The method of claim 89, wherein the one or more signature genes comprises a gene that indicates the abundance of macrophages in the tumor.

108. The method of claim 107, wherein the one or more signature genes is detected in CAFs.

109. The method of claim 108, wherein the one or more signature genes comprises C1S, C1R, CFB or HSD11B1.

110. The method of claim 107, wherein the one or more signature genes is detected in endothelial cells.

111. The method of claim 110, wherein the one or more signature genes comprises PECAM1, LMO2, or IL3RA.

112. The method of claim 107, wherein the one or more signature genes is detected in melanoma cells.

113. The method of claim 112, wherein the one or more signature genes comprises ceruloplasmin (CP).

114. The method of claim 89, wherein the one or more signature genes comprises a gene that indicates the functional state of a T cell from the tumor.

115. The method of claim 114, wherein the T cell comprises a Treg cell.

116. The method of claim 115, wherein the one or more signature genes comprises a signature gene of Table 12.

117. The method of claim 116, wherein the one or more signature genes comprises FOXP3 or IL2RA.

118. The method of claim 89, wherein the one or more signature genes comprises a gene that indicates the exhaustion state of an immune cell of the tumor.

119. The method of claim 118, wherein the one or more signature genes comprises a signature gene of Table 13, or Table 14.

120. The method of claim 119, wherein the one or more signature genes comprises PDCD1, TIGIT, HAVCR2, SIT1, LAG3, CTLA4, FAM3C, TNFRSF9, SYT11, GUSBP3, SIRPG, LY6E, CXCL13, SUMO2, IL2RG, CD74, CBLB, FOXN3, SLA, FKBP1A, CD27, SP100, IK, CCL3, CXCL13, TNFRSF1B, RGS2, RNF19A, INPP5F, XCL2, HLA-DMA, UQCRC1, WARS, EIF3L, KCNK5, TMBIM6, CD200, ZC3H7A, SH2D1A, ATP1B3, MYO7A, THADA, PARK7, EGR2, FDFT1, CRTAM, IFI16, LAG3, NFATC1, TIM3, PD-1, BTLA or CBLB.

121. The method of claim 78, wherein the one or more signature genes comprises a signature gene that indicates cell cycle state.

122. The method of claim 121, wherein the one or more signature genes is an indicator of a low-cycling or a high-cycling tumor.

123. The method of claim 122, wherein the one or more signature genes comprises cyclin D3 (CCND3) or KDM5B (JARID1B), wherein CCND3 indicates high-cycling tumors and KDM5B indicates non-cycling cells.

124. The method of claim 78, wherein the one or more signature gene(s) comprises a complement system gene.

125. The method of claim 124, wherein the one or more signature genes comprises C1S, C1R, C3, C4A, CFB or SERPING1.

126. The method of claim 78, wherein the one or more signature genes comprises a signature gene that is an indication of drug resistance.

127. The method of claim 78, wherein the level or expression of the one or more signature genes is determined by single-cell RNA sequencing.

128. The method of claim 78, wherein level of expression, activity and/or function of one or more signature genes is determined by the level of expression of one or more products encoded by one or more signature genes in one or more cell(s) of the melanoma.

129. The method of claim 128, wherein the level of expression of one or more products encoded by one or more signature genes is determined by a colorimetric assay or absorbance assay.

130. The method of claim 78, wherein level of expression, activity and/or function of one or more signature genes or one or more products of one or more signature genes in one or more cell(s) of the melanoma is determined by deconvolution of the bulk expression properties of a tumor.

131. A method for monitoring a subject undergoing a treatment or therapy for a melanoma comprising detecting a level of expression, activity and/or function of one or more signature genes or one or more products of one or more signature genes of the melanoma in the absence of the treatment or therapy and comparing the level of expression, activity and/or function of one or more signature genes or one or more products of one or more signature genes in the presence of the treatment or therapy, wherein a difference in the level of expression, activity and/or function of one or more signature genes or one or more products of one or more signature genes in the presence of the treatment or therapy indicates whether the patient is responsive to the treatment or therapy.

132. The method of claim 131, wherein the treatment or therapy modulates expression of one or more signature genes that indicates the functional state of an immune cell from the tumor.

133. The method of claim 131, wherein the treatment or therapy modulates expression of one or more signature genes that indicates cell cycle state.

134. A method of treating melanoma or enhancing treatment of a melanoma, which comprises administering an agent that increases the function of one or more signature genes or one or more products of one or more signature genes in one or more cell(s) of the melanoma, wherein the one or more signature genes or one or more products of one or more signature genes comprises a signature gene corresponding to abundance of an immune cell.

135. The method of claim 134, wherein the one or more signature genes comprises a gene that indicates the abundance of T cells in the tumor.

136. The method of claim 135, wherein the one or more signature genes comprises a signature gene of Table 15.

137. The method of claim 135, wherein the one or more signature genes is detected in CAFs.

138. The method of claim 137, wherein the one or more signature genes comprises CXCL12, CCL19, PD-L2, C1S, C1R, C3, C4A, CFB, HSD11B1, RARRES1, TMEM176A, TMEM176B or SERPING1.

139. The method of claim 135, wherein the one or more signature genes is detected in macrophages.

140. The method of claim 139, wherein the one or more signature genes comprises C1QA, C1QB or C1QC.

141. The method of claim 135, wherein the one or more signature genes is detected in endothelial cells.

142. The method of claim 141, wherein the one or more signature genes comprises PECAM1, LMO2, KIF19, IL3RA, RBP5, GP1BA, HAPLN3 or RSPO3.

143. The method of claim 135, wherein the one or more signature genes is detected in melanoma cells.

144. The method of claim 143, wherein the one or more signature genes comprises ceruloplasmin (CP).

145. The method of claim 134, wherein the one or more signature genes comprises a gene that indicates the abundance of B cells in the tumor.

146. The method of claim 145, wherein the one or more signature genes is detected in CAFs.

147. The method of claim 146, wherein the one or more signature genes comprises CCL19, CLU, C7, KEL, C3, HSD11B1, RAI2, ABI3BP or CDX1.

148. The method of claim 145, wherein the one or more signature genes is detected in endothelial cells.

149. The method of claim 148, wherein the one or more signature genes comprises RBP5, ART4, GP1BA, or PKHD1L1.

150. The method of claim 145, wherein the one or more signature genes is detected in melanoma cells.

151. The method of claim 150, wherein the one or more signature genes comprises ceruloplasmin (CP).

152. The method of claim 134, wherein the one or more signature genes comprises a gene that indicates the abundance of macrophages in the tumor.

153. The method of claim 152, wherein the one or more signature genes is detected in CAFs.

154. The method of claim 153, wherein the one or more signature genes comprises C1S, C1R, CFB or HSD11B1.

155. The method of claim 152, wherein the one or more signature genes is detected in endothelial cells.

156. The method of claim 155, wherein the one or more signature genes comprises PECAM1, LMO02, or IL3RA.

157. The method of claim 152, wherein the one or more signature genes is detected in melanoma cells.

158. The method of claim 157, wherein the one or more signature genes comprises ceruloplasmin (CP). The method of claim 138, wherein the one or more signature genes comprises CXCL12 or CCL19.

159. A method of treating melanoma or enhancing treatment of a melanoma, which comprises administering an agent that increases the function of one or more signature genes or one or more products of one or more signature genes in one or more cell(s) of the melanoma, wherein the one or more signature genes or one or more products of one or more signature genes comprises a signature gene of Table 12.

160. A method of treating melanoma or enhancing treatment of a melanoma, which comprises administering an agent that decreases the function of one or more signature genes or one or more products of one or more signature genes in one or more cell(s) of the melanoma, wherein the one or more signature genes or one or more products of one or more signature genes comprises a signature gene of Table 13, or Table 14.

161. The method of claim 160, wherein the one or more signature genes comprises PDCD1, TIGIT, HAVCR2, SIT, LAG3, CTLA4, FAM3C, TNFRSF9, SYT11, GUSBP3, SIRPG, LY6E, CXCL13, SUMO2, IL2RG, CD74, CBLB, FOXN3, SLA, FKBP1A, CD27, SP100, IK, CCL3, CXCL13, TNFRSF1B, RGS2, RNF19A, INPP5F, XCL2, HLA-DMA, UQCRC1, WARS, EIF3L, KCNK5, TMBIM6, CD200, ZC3H7A, SH2D1A, ATP1B3, MYO7A, THADA, PARK7, EGR2, FDFT1, CRTAM, IFI16, LAG3, NFATC1, TIM3, PD-1, BTLA or CBLB.

162. The method of claim 161, wherein the agent inhibits SIT, SIRPG, or CBLB.

163. A method of treating melanoma or enhancing treatment of a melanoma, which comprises administering an agent that modulates the activity and/or expression of one or more signature genes or one or more products of one or more signature genes in one or more cell(s) of the melanoma, wherein the one or more signature genes or one or more products of one or more signature genes is a complement system gene or gene product.

164. The method of claim 163, wherein the agent enhances the activity and/or expression of C1S, C1R, C3, C4A, CFB, C1QA, C1QB, or C1QC.

165. The method of claim 164, wherein the agent comprises a CRISPR-Cas system that activates expression of a complement system gene.

166. The method of claim 163, wherein the agent targets a complement defense gene selected from the group consisting of CD46, CD55, and CD59.

167. The method of claim 166, wherein the agent comprises a CRISPR-Cas system that targets the complement defense gene, whereby the gene is knocked out or expression is decreased.

168. The method of claim 163, wherein the agent is a natural product, whereby the complement system is activated in a tumor.

169. The method of claim 168, wherein the agent comprises a metalloproteinase, whereby complement system components are directly cleaved in a tumor.

170. The method of claim 168, wherein the agent comprises a serine protease, whereby complement system components are directly cleaved in a tumor.

171. A method of identifying at least one tumor specific T Cell receptor (TCR) for use in adoptive cell transfer, said method comprising:
(e) identifying by sequencing, TCRs from single tumor infiltrating T cells obtained from a tumor sample;
(f) selecting the TCRs that are clonal and/or are derived from a T cell that expresses one or more signature genes of exhaustion; and
(g) cloning the selected TCRs into a non-naturally occurring vector.

172. The method of claim 171, wherein the one or more signature genes of exhaustion comprises PDCD1, TIGIT, HAVCR2, SIT1, LAG3, CTLA4, FAM3C, TNFRSF9, SYT11, GUSBP3, SIRPG, LY6E, CXCL13, SUMO2, IL2RG, CD74, CBLB, FOXN3, SLA, FKBP1A, CD27, SP00, IK, CCL3, CXCL13, TNFRSF1B, RGS2, RNF19A, INPP5F, XCL2, HLA-DA, UQCRC1, WARS, EIF3L, KCNK5, TMBIM6, CD200, ZC3H7A, SH2D1A, A7P1B3, MYO7A, THADA, PARK7, EGR2, FDFT1, CRTAM, IFI16, LAG3, NFATC1, TIM3, PD-1, BTLA or CBLB.

173. A method of treating a subject in need thereof suffering from cancer comprising administering at least one activated T cell to the subject expressing at least one TCR pair identified by the method according to claim 171.

174. A non-naturally occurring T cell expressing a tumor specific TCR pair identified by the method according to claim 171.

175. A personalized cancer treatment for a patient in need thereof comprising:
(h) determining clonality of TCRs in tumor infiltrating T cells from the patient, and/or
(i) detecting expression of one or more signature genes for exhaustion, and/or
(j) detecting expression of one or more signature genes correlated to T cell abundance; and
(k) administering an agent that stimulates the patients preexisting immune response if (i) at least one clonal TCR is determined and/or (ii) one or more signature genes for exhaustion is detected and/or (iii) one or more signature genes correlated to T cell abundance is detected.

176. The personalized cancer treatment of claim 175, wherein the clonality and/or expression of one or more signature genes is detected by single cell RNA sequencing.

177. The method of claim 176, wherein the single-cell RNA sequencing comprises single nucleus RNA-Seq.

178. The personalized cancer treatment of claim 175, wherein the agent is a checkpoint inhibitor.
